(19) United States
(12) Patent Application Publication
(10) Pub. No.: US 2022/0401460 A1
(43) Pub. Date: Dec. 22, 2022

(54) MODULATING RESISTANCE TO BCL-2 INHIBITORS

(71) Applicant: Dana-Farber Cancer Institute, Inc., Boston, MA (US)

(72) Inventors: Catherine J. Wu, Boston, MA (US); Romain Guieze, Boston, MA (US)

(21) Appl. No.: 17/284,111

(22) PCT Filed: Oct. 10, 2019
(86) PCT No.: PCT/US2019/055702

§ 371 Date: Apr. 9, 2021

Related U.S. Application Data
(60) Provisional application No. 62/744,081, filed on Oct. 10, 2018.

(51) Int. Cl. A61K 31/635; A61K 31/519; A61K 31/145; A61K 31/4178; A61K 39/395; A61K 31/444; A61K 31/352; A61K 31/277; A61K 38/46; A61K 38/50; A61K 31/7105; A61P 35/00

(52) U.S. Cl. CPC A61K 31/635; A61K 31/519; A61K 31/145; A61K 31/4178; A61K 39/3955; A61K 31/444; A61K 48/00; A61K 31/277; A61K 38/465; A61K 38/50; A61K 31/7105; A61P 35/00; A61K 31/352

(57) ABSTRACT

This invention relates to compositions and methods for identifying the network that modulates, controls, or otherwise influences BCL-2 pathway inhibition, for example, energy-stress signaling, mitochondrial metabolism, vesicle transport, ribosomal components, and proteolysis. The invention also relates to identifying and modulating target genes and/or target gene products that modulate, control, or otherwise influence BCL-2 pathway inhibition.

### CROSS-REFERENCE TO RELATED APPLICATIONS

This application claims the benefit of U.S. Provisional Application No. 62/744,081, filed Oct. 10, 2018. The entire contents of the above-identified application are hereby fully incorporated herein by reference.

### STATEMENT REGARDING FEDERALLY SPONSORED RESEARCH

This invention was made with government support under Grant No. CA206978 awarded by the National Institutes of Health. The government has certain rights in the invention.

### REFERENCE TO AN ELECTRONIC SEQUENCE LISTING

The contents of the electronic sequence listing (BROD_4430_ST25.txt”; Size is 4,955 bytes and it was created on Oct. 10, 2019) is herein incorporated by reference in its entirety.

### TECHNICAL FIELD

The subject matter disclosed herein is generally directed to compositions and methods for identifying the network that modulates, controls, or otherwise influences BCL-2 pathway inhibition, for example, energy-stress signaling, mitochondrial metabolism, vesicle transport, ribosomal components, and proteolysis. The invention also relates to identifying and modulating target genes, target gene products and/or target pathways that modulate, control, or otherwise influence resistance to BCL-2 pathway inhibition.

### BACKGROUND

The B-cell lymphoma 2 (BCL-2) family includes both pro- and anti-apoptotic proteins that govern mitochondrial apoptosis. In leukemias and solid cancers, apoptosis dysregulation can result from overexpression of the anti-apoptotic BCL-2 protein that can sequester certain pro-apoptotic BH3-only proteins (BIM, BID) to avoid BAX and BAK oligomerization and subsequent mitochondrial outer membrane permeabilization. Within B cell tumors, BCL-2 dysregulation commonly arises from genetic abnormalities such as the translocation t(14;18)(q32;q21), which places BCL2 under the control of IGH promoter (in follicular lymphoma)^{1,2}; or focal deletion of chromosome 13 (del[13q14]), which leads to loss of a negative regulatory microRNA of BCL-2, miR-15a 16-1 (in chronic lymphocytic leukemia (CLL))³. Thus, BCL-2 has been a rational therapeutic target in lymphoid cancers.

Venetoclax (formerly ABT-199/GDC-0199) is a first-in-class BCL-2 inhibitor and has been recently FDA-approved for the treatment of CLL⁴. It displaces pro-apoptotic BH3-only proteins from BCL-2, allowing them to activate the mitochondrial pore-forming proteins BAK or BAX⁵. Despite its potent clinical activity in CLL cases failing control with chemotherapy regimens such as those carrying disruption of TP53⁴, disease progression on venetoclax is becoming an increasing therapeutic challenge^{6,7}.

Citation or identification of any document in this application is not an admission that such a document is available as prior art to the present invention.

### SUMMARY

In one aspect, the present invention provides for a method of inhibiting tumor growth of a BCL-2-driven cancer in a subject in need thereof comprising administering to the subject one or more agents capable of inhibiting the oxidative phosphorylation system (OXPHOS). In certain embodiments, the method comprises administering to the subject a combination therapy comprising an inhibitor of BCL-2 and one or more inhibitors selected from the group consisting of an AMPK inhibitor and mitochondrial electron transport chain (mETC) inhibitor. In certain embodiments, the BCL-2 inhibitor is venetoclax. In certain embodiments, the AMPK inhibitor is dorsomorphin (compound C). In certain embodiments, the mitochondrial electron transport chain (mETC) inhibitor comprises oligomycin or antimycin.

In another aspect, the present invention provides for a method of inhibiting tumor growth of a BCL-2-driven cancer in a subject in need thereof, the method comprising administering to said subject a therapeutically effective amount of one or more agents that induces or enhances expression, activity, and/or function of one or more BCL-2 inhibitor resistance signature genes selected from the group consisting of those listed in Table 1, downregulated genes in Table 3, and/or downregulated genes in Table 4; or an agent that inhibits expression, activity, and/or function of one or more BCL-2 inhibitor resistance signature genes selected from the group consisting of those listed in Table 2, upregulated genes in Table 3, and/or upregulated genes in Table 4. In certain embodiments, the agent increases expression, activity, and/or function of one or more target genes or one or more products of one or more target genes selected from the group consisting of: PMAIP1, BAX, NFKBIA, IKZF5, BAK1, ID3, EP300, ZEB2, NFIA, BCL2L11 and OTUD5; or FNBP1, CD9, PLXNB2, TTC39C and DENND3; or XBP1, CYBB, PAG1 and DIRAS1; or CD9, PLXNB2, TTC39C, DENND3, ICAM1, GNG7, ID2, FNBP1, FBP1, ACY3, CDKN1A, GALM, PTK2 and CYBB. In certain embodiments, the agent decreases expression, activity, and/or function of one or more target genes or one or more products of one or more target genes selected from the group consisting of: BCL2L1, BCL2L2, BCL2, MCL1, SRPX, RNF26, HSPB9, OR1S2, ADIPOQ, PIGF, CSGALNACT1, OTUD6A, SLC25A3, PRKAR2B, DNM2, SPHAR, APOBEC3C, RPL17, INMT, THADA, SBNO2, PRKAA2, BRMS1L, TRNAU1AP, CNNM3, ADAM33, PRKD2, FCHSD2, LOC399886, BABAM1, C1orf146, LMAN2L, ZNF460, TEX2, YRDC, ARHGAP11A, SPEG, FBXO9, USP54, SLC22A6, RPS4Y1, FAM71C, SH3BGRL2, HCRTR1, BST1, PHF10, UCKL1, ATG5, RPS15A, CDC20B, PPIE, TUT1, RPL36, HSD11B1L, MTERF4, PTS, S1PR4, HJURP, HMMR, BOLA2, DNASE1L1, OSGEP, TMBIM4, BTNL3, CHRM3, FBX015, KLK8, ASPN, STYK1 and SRSF6; or SYT11, PARM1, ROBO2, CD48, FCRL1 and MCL1; or PLCL2, KCNA3, TNFRSF21, CYP2U1, TRAM2 and RAPGEF5; or TSTD1, DNAJC12, TRAF3IP3, OPTN, DOCK10, PYHINI, CD48, P4HA2, PLCL2, AOX1, CDK6, GATM, GLUL, PAPSS1, MCL1 and GATM.

In certain embodiments, the tumor overexpresses BCL-2. In certain embodiments, the tumor is resistant to an inhibitor of BCL-2. In certain embodiments, the tumor is resistant to venetoclax. In certain embodiments, the method further comprises administering to said subject a therapeutically effective amount of an inhibitor of BCL-2. In certain embodiments, the inhibitor of BCL-2 is venetoclax.

In another aspect, the present invention provides for a method of inhibiting tumor growth of a BCL-2-driven cancer in a subject in need thereof comprising administering to the subject a combination therapy comprising an inhibitor of BCL-2 and one or more NF kappa B inhibitors. In certain embodiments, the NF kappa B inhibitor is selected from the group consisting of denosumab, disulfiram, olmesartan, dithiocarbamates, anatabine, BAY 11-7082 and iguratimod.

In another aspect, the present invention provides for a method of increasing sensitivity of a cell or population of cells to a BCL-2 inhibitor or decreasing a BCL-2 inhibitor resistance signature of a cell or population of cells, comprising contacting the cell or population of cells with one or more agents that enhance expression, activity, and/or function of one or more BCL-2 inhibitor resistance signature genes selected from the group consisting of those listed in Table 1, downregulated genes in Table 3, and/or downregulated genes in Table 4; or decrease expression, activity, and/or function of one or more BCL-2 inhibitor resistance signature genes selected from the group consisting of those listed in Table 2, upregulated genes in Table 3, and/or upregulated genes in Table 4. In certain embodiments, the one or more agents enhance expression, activity, and/or function of at least one gene selected from the group consisting of: PMAIP1, BAX, NFKBIA, IKZF5, BAK1, ID3, EP300, ZEB2, NFIA, BCL2L11 and OTUD5; or FNBP1, CD9, PLXNB2, TTC39C and DENND3; or XBP1, CYBB, PAG1 and DIRAS1; or CD9, PLXNB2, TTC39C, DENND3, ICAM1, GNG7, ID2, FNBP1, FBP1, ACY3, CDKN1A, GALM, PTK2 and CYBB. In certain embodiments, the one or more agents decrease expression activity, and/or function of at least one gene selected from the group consisting of: BCL2L1, BCL2L2, BCL2, MCL1, SRPX, RNF26, HSPB9, OR1S2, ADIPOQ, PIGF, CSGALNACT1, OTUD6A, SLC25A3, PRKAR2B, DNM2, SPHAR, APOBEC3C, RPL17, INMT, THADA, SBNO2, PRKAA2, BRMS1L, TRNAU1AP, CNNM3, ADAM33, PRKD2, FCHSD2, LOC399886, BABAM1, C1orf146, LMAN2L, ZNF460, TEX2, YRDC, ARHGAP11A, SPEG, FBXO9, USP54, SLC22A6, RPS4Y1, FAM71C, SH3BGRL2, HCRTR1, BST1, PHF10, UCKL1, ATG5, RPS15A, CDCl20B, PPIE, TUT1, RPL36, HSD11B1L, MTERF4, PTS, S1PR4, HJURP, HMMR, BOLA2, DNASE1L1, OSGEP, TMBIM4, BTNL3, CHRM3, FBX015, KLK8, ASPN, STYK1 and SRSF6; or SYT11, PARM1, ROBO2, CD48, FCRL1 and MCL1; or PLCL2, KCNA3, TNFRSF21, CYP2U1, TRAM2 and RAPGEF5; or TSTD1, DNAJC12, TRAF3IP3, OPTN, DOCK10, PYHINI, CD48, P4HA2, PLCL2, AOX1, CDK6, GATM, GLUL, PAPSS1, MCL1 and GATM. In certain embodiments, the one or more agents enhance expression, activity, and/or function of one or more genes selected from the group consisting of: PMAIP1, BAX, BAK1, or BCL-2L11, NFKBIA, IKZF5, ID3, EP300, NFIA, OTUD5, or UBR5; or FNBP1, CD9, PLXNB2, TTC39C, DENND3, XBP1, CYBB, PAG1, DIRAS1, ICAM1, GNG7, ID2, FBP1, ACY3, CDKN1A, GALM or PTK2; or decrease expression, activity, and/or function of one or more genes selected from the group consisting of: BCL2L1, BCL2L12, BCL2 or MCL1, ADIPOQ, PRKAR2B, PRKAA2, SLC25A3, RFN26, DNM2, PRKD2, ATG5, RPL17, RPS4Y1, RPS15A, OUTUD6A, FBXO9, or USP54, or SYT11, PARM1, ROBO2, CD48, FCRL1, MCL1, PLCL2, KCNA3, TNFRSF21, CYP2U1, TRAM2, RAPGEF5, TSTD1, DNAJC12, TRAF3IP3, OPTN, DOCK10, PYHINI, CD48, P4HA2, AOX1, CDK6, GATM, GLUL, PAPSS1 or GATM.

In another aspect, the present invention provides for a method of screening for one or more agents that increases a BCL-2 inhibitor sensitive signature or decreases a BCL-2 inhibitor resistance signature of a cell or a population of cells that expresses BCL-2 comprising: delivering to the cell one or more candidate agents and selecting one or more agents that: a) increases expression, activity, and/or function of one or more target genes or one or more products of one or more genes selected from the group consisting of those listed in Table 1, downregulated genes in Table 3, and/or downregulated genes in Table 4; or b) decreases expression, activity, and/or function of one or more target genes or one or more products of one or more target genes selected from the group consisting of those listed in Table 2, upregulated genes in Table 3, and/or upregulated genes in Table 4. In certain embodiments, the one or more candidate agents increase expression, activity, and/or function of one or more target genes or one or more products of one or more target genes which comprise inhibitors of the NF-Kappa B pathway, lymphoid transcription factors and modulators, ubiquitination components, and/or pro-apoptotic BCL-2 family proteins. In certain embodiments, the one or more candidate agents decrease expression, activity, and/or function of one or more target genes or one or more products of one or more target genes which comprise energy-stress sensor signaling pathway components, a mitochondrial energy metabolism component, vesicle transport/autophagy components, ribosomal components, and/or ubiquitination components. In certain embodiments, the one or more candidate agents increase expression, activity, and/or function of one or more target genes or one or more products of one or more target genes selected from the group consisting of: PMAIP1, BAX, NFKBIA, IKZF5, BAK1, ID3, EP300, ZEB2, NFIA, BCL2L11 and OTUD5; or FNBP1, CD9, PLXNB2, TTC39C and DENND3; or XBP1, CYBB, PAG1 and DIRAS1; or CD9, PLXNB2, TTC39C, DENND3, ICAM1, GNG7, ID2, FNBP1, FBP1, ACY3, CDKN1A, GALM, PTK2 and CYBB. In certain embodiments, the one or more candidate agents decrease expression, activity, and/or function of one or more target genes or one or more products of one or more target genes selected from the group consisting of: BCL2L1, BCL2L2, BCL2, MCL1, SRPX, RNF26, HSPB9, OR1S2, ADIPOQ, PIGF, CSGALNACT1, OTUD6A, SLC25A3, PRKAR2B, DNM2, SPHAR, APOBEC3C, RPL17, INMT, THADA, SBNO2, PRKAA2, BRMS1L, TRNAU1AP, CNNM3, ADAM33, PRKD2, FCHSD2, LOC399886, BABAM1, C1orf146, LMAN2L, ZNF460, TEX2, YRDC, ARHGAP11A, SPEG, FBXO9, USP54, SLC22A6, RPS4Y1, FAM71C, SH3BGRL2, HCRTR1, BST1, PHF10, UCKL1, ATG5, RPS15A, CDC20B, PPIE, TUT1, RPL36, HSD11B1L, MTERF4, PTS, S1PR4, HJURP, HMMR, BOLA2, DNASE1L1, OSGEP, TMBIM4, BTNL3, CHRM3, FBX015, KLK8, ASPN, STYK1 and SRSF6; or SYT11, PARM1, ROBO2, CD48, FCRL1 and MCL1; or PLCL2, KCNA3, TNFRSF21, CYP2U1, TRAM2 and RAPGEF5; or TSTD1, DNAJC12, TRAF3IP3, OPTN, DOCK10, PYHINI, CD48, P4HA2, PLCL2, AOX1, CDK6, GATM, GLUL, PAPSS1, MCL1 and GATM. In certain embodiments, the cell or population of cells overexpresses BCL-2. In certain embodiments, the method further comprises exposing the cell or population of cells to an agent that modulates the expression or activity of at least one BCL-2 antagonist of cell death (BAD) pathway component. In certain embodiments, the method further comprises exposing the cell or population of cells to an agent that inhibits BCL-2. In certain embodiments, the agent that inhibits BCL-2 is venetoclax.

In certain embodiments, the agent is a small molecule, small molecule degrader, genetic modifying agent, antibody, antibody fragment, antibody-like protein scaffold, aptamer, protein, or any combination thereof. In certain embodiments, the genetic modifying agent comprises a CRISPR system, RNAi system, a zinc finger nuclease system, a TALE system, or a meganuclease. In certain embodiments, the CRISPR system comprises a Class 2, Type II, V, or VI CRISPR-Cas system. In certain embodiments, the CRISPR system comprises a dCas fused or otherwise linked to a nucleotide deaminase. In certain embodiments, the nucleotide deaminase is a cytidine deaminase or an adenosine deaminase.

In another aspect, the present invention provides for a method of detecting a BCL-2 inhibitor resistance signature in a subject in need thereof comprising detecting in a tumor sample obtained from the subject the expression of one or more genes selected from the group consisting of those listed in Table 1, Table 2, Table 3, and/or Table 4. In certain embodiments, the genes selected from the group consisting of: PMAIP1, BAX, NFKBIA, IKZF5, BAK1, ID3, EP300, ZEB2, NFIA, BCL2L11 and OTUD5; or FNBP1, CD9, PLXNB2, TTC39C and DENND3; or XBP1, CYBB, PAG1 and DIRAS1; or CD9, PLXNB2, TTC39C, DENND3, ICAM1, GNG7, ID2, FNBP1, FBP1, ACY3, CDKN1A, GALM, PTK2 and CYBB, are downregulated as compared to a reference value. In certain embodiments, the genes selected from the group consisting of: BCL2L1, BCL2L2, BCL2, MCL1, SRPX, RNF26, HSPB9, OR1S2, ADIPOQ, PIGF, CSGALNACT1, OTUD6A, SLC25A3, PRKAR2B, DNM2, SPHAR, APOBEC3C, RPL17, INMT, THADA, SBNO2, PRKAA2, BRMS1L, TRNAU1AP, CNNM3, ADAM33, PRKD2, FCHSD2, LOC399886, BABAM1, C1orf146, LMAN2L, ZNF460, TEX2, YRDC, ARHGAP11A, SPEG, FBXO9, USP54, SLC22A6, RPS4Y1, FAM71C, SH3BGRL2, HCRTR1, BST1, PHF10, UCKL1, ATG5, RPS15A, CDCl20B, PPIE, TUT1, RPL36, HSD11B1L, MTERF4, PTS, S1PR4, HJURP, HMMR, BOLA2, DNASE1L1, OSGEP, TMBIM4, BTNL3, CHRM3, FBX015, KLK8, ASPN, STYK1 and SRSF6; or SYT11, PARM1, ROBO2, CD48, FCRL1 and MCL1; or PLCL2, KCNA3, TNFRSF21, CYP2U1, TRAM2 and RAPGEF5; or TSTD1, DNAJC12, TRAF3IP3, OPTN, DOCK10, PYHINI, CD48, P4HA2, PLCL2, AOX1, CDK6, GATM, GLUL, PAPSS1, MCL1 and GATM, are upregulated as compared to a reference value. In certain embodiments, if a BCL-2 inhibitor resistance signature is detected the method further comprises administering a treatment to the subject according to any embodiment herein.

In another aspect, the present invention provides for a method of identifying a signature gene, a gene signature, or other genetic element associated with a BCL-2 family function, activity or phenotype comprising: a) contacting a cell or population of cells with an agent that inhibits an anti-apoptotic BCL-2 family protein or a gene that encodes the protein; and b) identifying one or more gene loci whose activity is modulated by step (a); thereby identifying a signature gene, a gene signature, or other genetic element associated with a BCL-2 family function. In certain embodiments, the cell or population of cells comprises a Cas protein or nucleic acid encoding the Cas protein and one or more guides or nucleic acids encoding the one or more guides, wherein the guide(s) target one or more nucleic acid(s) in the cell or population of cells, whereby one or more nucleic acid(s) in the cell or population of cells is modified, whereby the viability of a cell or population of cells comprising the one or more modified nucleic acid(s) is modulated. In certain embodiments, the cell or population of cells comprises nucleic acids modified by a CRISPR-Cas system comprising a Cas protein and one or more guides. In certain embodiments, the viability of the cell or cell population comprising the one or more modified nucleic acid(s) is correlated with representation of one or more of the one or more guides. In certain embodiments, the cell or population of cells comprises one or more gene knock-outs. In certain embodiments, the CRISPR-Cas system comprises a Cas9. In certain embodiments, the BCL-2 family protein is BCL-2.

In another aspect, the present invention provides for a kit comprising reagents to detect at least one gene or gene product according to any of the preceding claims.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1I—Orthogonal genome-wide screens for genes driving venetoclax resistance. a, Experimental schema of the parallel knockout and overexpression screens using the BCL-2 driven OCI-Ly1 cell line (two biologically independent experiments for each screen). b-c, sgRNAs and ORFs frequencies, respectively, at different timepoints during the screens (two independent experiments shown), black bars are mean+/−s.d., two-sided t-test. d-e, Scatter plots showing the average log 2fold-change (LFC) for each gene in both duplicates of the loss-of-function and gain-of-function screens, respectively (only genes with LFC>−1 are shown). Genes with a significant increase of sgRNAs representation (using the gene-ranking algorithm STARS, Broad Institute) and within the top-50 ORFs are highlighted. f, Dose-response curves to venetoclax of 2 representative knockout OCI-Ly1 cells with related western-blots for quantification of the target protein. g, Differential expression of eight genes. h, Cumulative growth over time of each of the genetically perturbed OCI-Ly1 cells. i, PKA and AMPK are central regulators of cellular energy metabolism.

FIG. 2—Expression changes related to acquisition of venetoclax resistance and MCL-1 targeting. a, Dose-response curve of both the generated drug-resistant (OCI-Ly1-R) and the drug-sensitive parental cell line (OCI-Ly1-S). b, Scatter plot reporting log 2fold-change (LFC) of both transcript (X-axis) and protein (Y-axis) levels between OCI-Ly1-S and OCI-Ly1-R cells. Red label indicates adjusted P-value <0.05 at the protein level (see Methods). c, Western-blot showing MCL-1, BCL-XL and BCL-2 proteins expression in OCI-Ly1-S and OCI-Ly1-R cells. d, Dose-response curves of OCI-Ly1-S to venetoclax and varying doses of the MCL-1 inhibitor S63845 (5, 10 and 100 nM). e, Combination index according to the fraction affected (top) and normalized isobologram (bottom), Chou-Talalay method (see Methods). f, Viability of the OCI-Ly1-R line 24 hours after exposure to venetoclax 100 nM, S63845 50 nM and both drugs (and DMSO as control), data are mean+/−s.e.m. from three biologically independent experiments, P-value is from ANOVA test with adjustment for multiple comparisons. g, Relevant gene set enrichment plots based on differential RNA expression changes between OCI-Ly1-S and OCI-Ly1-R.

FIG. 3—Investigating oxidative phosphorylation in the venetoclas resistant OCI-Ly1 cells. A diagram of the Seahorse assay described below.

FIG. 4—Metabolic changes associated with resistance to BCL-2 inhibition. a, Oxygen consumption rate over time in both OCI-Ly1-S and OCI-Ly-1-R lines upon the use of inhibitors to derive parameters of mitochondrial respiration (Seahorse assay, see Methods). b, Histogram plot showing the ratio of mitochondrial DNA (mtDNA) over nuclear DNA (nucDNA) in both OCI-Ly1-S and OCI-Ly-1-R cells. c, Histogram plots highlighting quantification of the reactive oxygen species superoxide by flow cytometry in both OCI-Ly1-S and OCI-Ly-1-R cells. d, Oxygen consumption rate and e, Extracellular acidification rate over time in both OCI-Ly1-S and OCI-Ly-1-R lines upon the treatment by venetoclax, with or without prior zVAD treatment, or DMSO as control (representative experiment of 3 biological replicates). f, Dose-response curves of OCI-Ly1-S to venetoclax. The cell line has been exposed to increasing doses of the AMPK inhibitor dorsomorphin (left), the inhibitor of electron transport chain complex 3 antimycin (middle) and the F1Fo-ATPase inhibitor oligomycin in addition to venetoclax (right). g, Histogram plots showing the viability of the OCI-Ly1-R line after exposure to DMSO as control, venetoclax 100/500 nM with and without antimycin 10 nM or oligomycin 1 microM. Data are mean+/−s.e.m. from three biologically independent experiments (panel b and g) and one representative experiment of three biological replicates (panel a, d and e), * means P<0.0001. P-value is from two-sided two sample t-test.

FIG. 5—The resistance circuit related to ID3 repression implicates metabolism. a, Western-blot for quantification of MCL-1 in genetically perturbed OCI-Ly1 cell lines. b, Dose-response curves to the MCL-1 inhibitor S63845 of OCI-Ly1 cells engineered as indicated. c, Heatmap reporting genes differentially expressed at the RNA level between the OCI-Ly1-S and OCI-Ly1-R cells. d, Volcano plot showing transcripts changes in ID3 knockout OCI-Ly1 cells compared to non-targeting sgRNA transduced OCI-Ly1 cells. e, Western-blot for quantification of ID2 and ID3 proteins in PRKAR2B (PKA) and PRKAA2 (AMPK) overexpressing OCI-Ly1 cell lines. f, Histogram plots showing the viability at 24 hours of single-cell clones from ID3 knockout OCI-Ly1 cells compared to non-targeting sgRNAs transduced OCI-Ly1 cells after exposure to dorsomorphin and oligomycin in addition to venetoclax. Data are mean+/−s.d. from three biologically independent experiments and P-values are from ANOVA test.

FIG. 6—Clonal evolution in CLL patients developing resistance to venetoclax. a, Somatic copy number variations in both OCI-Ly1-S and OCI-Ly1-R cells. Red is gain and blue is loss. b, Subclonal composition and clonal evolution of 6 patients developing resistance to venetoclax. Driver mutations associated with each clone are indicated. c, Comparison (modal cancer cell fraction (CCF) with 95% CI) between pre-treatment and relapse samples for select drivers recurrently observed in CLL or in the setting of venetoclax resistance. d, Representation of the minimal gained region in the 1q locus across both the OCI-Ly1 cell lines and the patient samples. e, Proposed model for venetoclax resistance.

FIG. 7—Validation of gene hits from orthogonal genome-wide screens. a, Cumulative growth of cells during loss-of-function and gain-of-function screens. b, Log 2fold-change (LFC) of sgRNAs (4 per gene) for genes with significant change in representation during the loss-of-function screen (significance as determined by using the gene-ranking algorithm STARS, Broad Institute), horizontal line is mean and error bars indicate s.d. c, Scatter plots showing the average LFC for each gene in both duplicates of the loss-of-function screen with known pro-apoptotic proteins and anti-apoptotic proteins highlighted. d, Protein expression levels in single gene knockout isogenic cell lines (2 lines per gene), before and after selection with venetoclax. e, Western-blot for the target proteins (PRKAR2B [PKA] and PRKAA2 [AMPK]) in ORFs transduced OCI-Ly1 cells. f, Venetoclax IC50 fold change of single gene knockout isogenic cell lines compared to OCI-Ly1 cells transduced with control sgRNAs. Data are mean+/−s.e.m., *P from extra sum-of-squares F test. g, Frequency of frame-shift indels in single gene knockout isogenic cell lines before and after 2 weeks of venetoclax treatment.

FIG. 8—Metabolic changes associated with resistance to BCL-2 inhibition. a, RNA-sequencing of parental vs. venetoclax-resistant OCI-Ly1 cells, significantly dysregulated genes (adjusted P-value <0.05) with log 2fold change >2 indicated in red and log 2fold change <−2 indicated in blue. b, Evaluation of mitochondrial membrane potential using JC-1 staining in each of the OCI-Ly1-S and OCI-Ly1-R cells. Data are mean+/−s.e.m. from three replicates, P-value is from two-sided t-test. c, Analysis of synergism of venetoclax with antimycin, dorsomorphin, and oligomycin. Combination index according to the fraction affected (left) and normalized isobologram (right), Chou-Talalay method (see Methods). d, Western-blot showing ID3 expression in single-cell clones from Cas9 expressing OCI-Ly1 cells transduced with ID3 targeting shRNA. e, Sensitivity of AMPK and PKA overexpressing cells to venetoclax when used in combination with dorsomorphin (2 μM) and oligomycin (1 μM). Data are mean+/−s.e.m. from three biologically independent experiments, P-value is from two-sided t-test.

FIG. 9—Genomic investigations of OCI-Ly1 cells and primary CLL cells from patients developing resistance on venetoclax. a, Somatic copy number variations calling from WES data (AllelicCapseg plots and Absolute segmented plots) of OCI-Ly1 cells. b, Mutation burden in baseline and relapse samples. c, Bars plots related to subclonal composition inferred from cancer cell fraction (CCF) estimation using the ABSOLUTE algorithm (see Methods). Phylogenetic trees were built based on Absolute estimations. Driver mutations associated with each clone are indicated in Table 8. c, Comparison (modal cancer cell fraction (CCF) with 95% CI) between pre-treatment and relapse samples for selected drivers recurrently observed in CLL or in the setting of this study.

FIG. 10—Somatic copy number variations calling from patient WES data. AllelicCapseg plots and Absolute segmented plots of patient tumor samples before (Pre) and after venetoclax (Post).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to Guieze et al., Mitochondrial Reprogramming Underlies Resistance to BCL-2 Inhibition in Lymphoid Malignancies, Cancer Cell. 2019 Sep. 4. pii: S1535-6108(19)30373-3.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide the determinants of venetoclax resistance by using genome-scale survival screens, phenotypic characterization of venetoclax-resistant lymphoid cell lines, and exome-wide sequencing-based analysis of drug-resistant cell lines and primary CLL samples, discussed in further detail below. These complementary analyses revealed venetoclax resistance to involve not only modulation of BCL2-family members, but also broader changes in mitochondrial metabolism. The present invention provides BCL-2 inhibitor resistance gene signatures and target genes that confer BCL-2 inhibitor resistance. The BCL-2 inhibitor resistance signature(s) may be characterized by expression of the gene or gene products (see, Tables 1, 2, 3 and 4 herein).

Inhibition of apoptosis is regulated by the B-cell lymphoma 2 (BCL-2) family and is a hallmark of many cancers, including lymphoid malignancies. The first-in-class BCL-2 inhibitor venetoclax is transforming the treatment landscape of diverse malignancies, but resistance to this agent has emerged as a therapeutic challenge.

To systematically discover the determinants of venetoclax resistance, Applicants conducted parallel genome-scale knockout (loss-of-function (LOF)) (Table 1) and overexpression (gain-of-function (GOF)) (Table 2) genetic modifier screens of the BCL-2-driven OCI-Ly1 lymphoma cell line after venetoclax exposure. Applicants identified genes involved in regulation of lymphoid transcription (IKZF5, EP300, NFKBIA, ID3) and cellular metabolism (PKA, AMPK), and known BCL-2 family members.

Further, integrated transcriptome, proteome and functional characterization of candidate hits of an OCI-Ly1 cell line rendered resistant to venetoclax (OCI-Ly1-R) from the parental cell line (OCI-Ly1-S), and genetic characterization of tumor DNA from patients with venetoclax-resistant chronic lymphocytic leukemia implicate roles for both inner (oxidative metabolism) and outer membrane (MCL-1 expression) mitochondrial adaptation. The RNA-seq and spectrometry-based proteomics revealed coordinated dysregulation of transcripts (Table 3) and proteins (Table 4) in the resistant line originating from genes critical to cellular metabolism, cell cycle, B-cell biology and autophagy.

Of the transcripts and proteins significantly associated with the resistant cell line, only MCL-1 overlapped with the gene hits from the genome-scale screens. Treatment of the OCI-Ly-R cells with the MCL-1 inhibitor S63845 synergized with venetoclax. Given the dysregulation of proteins critical to metabolism in both the GOF screen and in OCI-Ly1-R cells, the role of metabolic reprogramming in venetoclax resistance was assessed, for example by measuring the oxygen consumption rate. Compared to OCI-Ly-S cells, OCI-Ly1-R cells demonstrated markedly higher respiration levels, suggesting a state of higher oxidative phosphorylation (OXPHOS). Direct measurement of oxygen consumption following venetoclax exposure, consistent with impairment of OXPHOS by venetoclax, demonstrates both an immediate decrease in oxygen consumption and an immediate burst of glycolysis following venetoclax in the OCI-Ly1-S cells, but not in the OCI-Ly1-R cells. In line with these findings, the AMPK inhibitor dorsomorphin and mitochondrial electron transport chain (mETC) inhibitors synergized with venetoclax in OCI-Ly1-S cells. A transcriptome related to ID3 (identified as one of the LOF screen targets) was characterized in isogenic ID3-knockout OCI-Ly1 lines. It revealed PRKAR2B overexpression as a key effect, indicating a role for ID3, and other lymphoid transcription factors in regulating metabolic reprogramming associated with resistance, and exposure of ID3 knockout lines to mETC inhibitors overcame resistance to venetoclax.

To determine if there is a genetic basis for the drug resistance seen in OCI-Ly1-R cells, whole-exome sequencing (WES) results of DNA isolated from the OCI-Ly1-R and OCI-Ly1-S cell lines were compared. A clear region was amplified on chromosome 1q23, which includes MCL1 and PRKAB2 (the regulatory subunit of AMPK). Similarly, a WES-based analysis of paired CLL DNA samples isolated from 6 R/R CLL patients just prior to venetoclax initiation and at time of progression on venetoclax was performed. In the patient sample, non-silent somatic single nucleotide mutations in BCL2 or its family members were not observed at baseline or at progression, despite marked clonal shifts in all patients, but the presence of the amp(1q23) as acquired at relapse after venetoclax was observed in 3 out of 6 patients.

Venetoclax resistance implicates changes not only for outer mitochondrial membrane (MCL-1 expression) but also for inner membrane (oxidative metabolism). Such mitochondrial reprogramming represents a new vulnerability that can potentially be exploited through combinatorial therapy with metabolic modulators to overcome resistance, including through combinatorial therapies with metabolic modulators, to overcome resistance.

Accordingly, embodiments disclosed herein provide methods for detecting BCL-2 inhibitor resistance signatures, methods for treating tumors characterized by BLC-2 inhibitor resistance, and methods of screening for and identifying therapeutic agents useful in treating BCL-2 inhibitor resistant tumors.

### Diagnostic Methods

### BCL-2 Responsive Genes, Signatures and Pathways

The invention provides methods and compositions and identified genome-scale loss-of-function (LOF) (Table 1) and gain-of-function (GOF) (Table 2) genetic modifiers of resistance to BCL-2 and BCL-2 family inhibitors, such as but not limited to venetoclax. The invention also provides for genes (Table 3) and gene products (Table 4) differentially expressed between BCL-2 inhibitor resistant and sensitive parental BCL-2 driven tumor cells. In certain embodiments, one or more target genes or one or more products of one or more target genes that have been identified as genes responsive to the BCL-2-related perturbations (loss or gain of function) are detected, such as for use as diagnostic targets. In certain embodiments, BCL-2 inhibitor resistant tumors have a lower overall survival or increased risk of not responding to any treatment (e.g., BCL-2 inhibition or standard chemotherapy). As used herein, “BCL-2 inhibitor sensitive” may refer to a pro-apoptotic cell or population of cells, an anti-proliferative cell or population of cells, or a cell or population of cells that is sensitive to treatment. In certain embodiments, BCL-2 inhibitor sensitive cells are sensitive to treatment with BCL-2 inhibitors (e.g., venetoclax, aka, Venclexta, Venclyxto, GDC-0199, ABT-199 and RG7601). As used herein, “BCL-2 inhibitor resistant” refers to a non-apoptotic cell or population of cells, a proliferative cell or population of cells, or a cell or population of cells that is resistant to treatment. In certain embodiments, BCL-2 inhibitor resistant cells are resistant to treatment with BCL-2 inhibitors (e.g., venetoclax, aka, Venclexta, Venclyxto, GDC-0199, ABT-199 and RG7601). A BCL-2 inhibitor resistant signature is a gene signature present in BCL-2 inhibitor resistant cells.

All gene name symbols refer to the gene as commonly known in the art. The examples described herein that refer to the human gene names are to be understood to also encompasses genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene, including the gene products (e.g., proteins). The signatures as described herein may encompass any of the genes described herein. In some embodiments, the one or more signature genes are selected from those listed in Tables 1, 2, 3 and 4 shown below.

TABLE 1
CRISPR-Cas9 LOF screen hits (STARS output): Gene symbols

| | | | |
|---|---|---|---|
| PMAIP1 | CACNA1D | AJUBA | FAM86C1 |
| BAX | ZNF706 | TUBB8 | PON3 |
| NFKBIA | DUSP7 | PMAIP1 | ZEB2 |
| IKZF5 | HMGB1 | BAX | FATE1 |
| BAK1 | NCK1 | NFKBIA | CCDC71L |
| ID3 | NUDT1 | IKZF5 | VPS13B |
| EP300 | GRHL3 | BAK1 | CXCR2 |
| ZEB2 | LGR6 | EP300 | IZUMO1 |
| NFIA | TRNP1 | BCL2L11 | SLC22A6 |
| BCL2L11 | LRRC47 | OTUD5 | ASNS |
| OTUD5 | HIPK1 | UBR5 | RAX |
| UBR5 | ZC3H6 | TMPRSS15 | TMEM47 |
| GALNT6 | RAB9B | NFIA | C3orf58 |
| FBXO11 | FOXE3 | REXO4 | YES1 |
| PRR12 | CUL4B | DTNB | BZW2 |
| FRMD7 | MYLK | C8orf44-SGK3 | CDK2AP2 |
| GDF1 | RUVBL1 | ID3 | FRAT1 |
| OR6C65 | QSNER1 | ALG13 | TFEB |
| RC3H1 | GAMT | ALG11 | SRP54 |
| TAF9 | KIF3C | VIP | YTHDF3 |
| FAM122A | BEND4 | JCHAIN | SPIN2A |
| BGLAP | BIK | ASAP2 | CPVL |
| ATOH1 | FTL | ATG9B | PDCL |
| SNX19 | FLG2 | STIP1 | TMEM190 |
| PDE5A | TRAF3 | KCNJ10 | CT47A5 |
| ARHGEF39 | PDZD8 | ARHGEF39 | DEFB108B |
| RAX | ITPKB | PABPN1 | RNF219 |
| CHRNA4 | ACOT8 | PRR12 | MED19 |
| HPR | GABRG3 | NR1I2 | LAMB3 |
| STIP1 | BEX4 | LTBP3 | ZFP30 |
| KLK15 | RWDD1 | OR51E2 | SLC5OA1 |
| FOXO1 | AZIN1 | AJUBA | FAM86C1 |

TABLE 2
ORF GOF screen hits

| | | | | | |
|---|---|---|---|---|---|
| BCL2L1 | BCL2L2 | BCL2 | MCL1 | SRPX | RNF26 |
| HSPB9 | OR1S2 | ADIPOQ | PIGF | CSGALNACT1 | OTUD6A |
| SLC25A3 | PRKAR2B | DNM2 | SPHAR | APOBEC3C | RPL17 |
| INMT | THADA | SBNO2 | PRKAA2 | BRMS1L | TRNAU1AP |
| CNNM3 | ADAM33 | PRKD2 | FCHSD2 | LOC399886 | BABAM1 |
| C1orf146 | LMAN2L | ZNF460 | TEX2 | YRDC | ARHGAP11A |
| SPEG | FBXO9 | USP54 | SLC22A6 | RPS4Y1 | FAM71C |
| SH3BGRL2 | HCRTR1 | BST1 | PHF10 | UCKL1 | ATG5 |
| RPS15A | CDC2OB | PPIE | TUT1 | RPL36 | HSD11B1L |
| MTERF4 | PTS | S1PR4 | HJURP | HMMR | BOLA2 |
| DNASE1L1 | OSGEP | TMBIM4 | BTNL3 | CHRM3 | FBXO15 |
| KLK8 | ASPN | STYK1 | SRSF6 | WWC2-AS2 | FAM84B |
| PTCD2 | C19orf47 | AADAC | TSSK6 | MED9 | NR2E3 |
| VCP | PMEPA1 | CCDC144A | DNAJB11 | BEX3 | PRDX3 |
| ZBTB9 | FASTK | BAAT | AASDHPPT | DHRS11 | MAPKAPK2 |
| XLOC_005244 | TMEM132A | TYROBP | PLGLB2 | ADK | TMEM151A |
| MED18 | KCTD16 | NPL | TM2D2 | LYG2 | GLIS3-AS1 |
| CFAP74 | SNRK | CGRRF1 | RRAGD | CTBP1-AS2 | TFPI2 |
| HACD1 | GMNN | PRRT2 | CCDC115 | CDKN2A | C2orf82 |
| CCK | PIAS1 | S100A4 | SNRNP48 | RUVBL2 | FAM234B |
| TOX2 | LGALS9 | BTLA | GDPD5 | LSAMP | XLOC_I2_015937 |
| C5orf49 | IL36A | LAPTM4A | EFCAB1 | GNAZ | RBP7 |
| NECAB1 | IGSF11 | CA5B | ERGIC2 | GNPDA1 | ARL16 |
| PPID | GPRC5B | FHIT | LTA4H | LOC107987001 | LYN |
| SIAE | STK32A | MTERF2 | MMP28 | COQ8B | TMEM147 |
| CRYGD | CD7 | PBRM1 | ZMYND10 | PIPOX | FAM53C |
| NOP56 | NOTCH2NL | IL10RB | SRSF10 | RHBDD1 | GLYCTK |
| MAX | GPR45 | FOS | DUSP19 | STAMBPL1 | TGM5 |
| TMEM131 | CELA2B | ZNF267 | HSPB8 | TAS2R10 | C1QTNF3 |
| VSX1 | CYCS | USP30 | C8orf4 | ABHD12 | SNX20 |
| RBBP6 | DIMT1 | RPUSD2 | IRF2 | ADAM18 | AURKA |
| CIAPIN1 | STAM2 | SLC38A1 | Sf3b3 | KHK | GTF2B |
| SLC36A4 | ACVR1C | OR10G2 | SLC12A9 | EMC4 | RAB40C |
| FABP5 | HIST2H3C | OR4F15 | ALDH1B1 | UGT1A9 | LOC554206 |
| CA8 | SCD5 | COMT | AP1S1 | CCL4 | CD86 |
| UBLCP1 | CAP2 | ABCA3 | HCP5 | PPP4C | LATS1 |
| H2AFJ | PKMYT1 | GAGE7 | FUZ | POPDC3 | MTFP1 |
| C2orf69 | TMEM133 | EFNB3 | C15orf39 | BIRC5 | SGK3 |
| ACTRT2 | KDELR2 | DOHH | CD2 | TFPI | KLK1 |
| POLR1D | BCOR | ATOH7 | SLC17A8 | FFAR4 | SFTA2 |
| SYCP1 | OR2A25 | AURKB | RET | CACUL1 | RBM43 |
| NME3 | RPLP2 | FIGN | MARK3 | DHCR24 | IL37 |
| NUFIP2 | GATA1 | APOBEC3D | AKR1C3 | PSMA8 | CAMTA2 |
| C5orf58 | UBE2J1 | SLAMF6 | LPAR3 | METTL21C | ITFG2 |
| TVP23A | C2 | TRMT112 | PDZD7 | CTSE | TMEM59 |
| C20orf166-AS1 | CT47A11 | FAM206A | FYTTD1 | SBNO1 | CCL3L1 |
| PADI1 | SLC25A31 | FCGR2A | HYPM | NEU2 | POLR1C |
| NPHP4 | C9orf62 | FCGR3B | XKR3 | BUB3 | TKFC |
| BPIFB3 | GPR65 | PHF6 | RCCD1 | TP53I11 | NDRG4 |
| TAS2R3 | THAP8 | RETN | GPR176 | LRRC8E | ZNF436-AS1 |
| ERC1 | ADH6 | DGKE | HIST1H2AL | ABHD17A | FTHL17 |
| NME2 | KBTBD4 | AXIN2 | TAGAP | CT83 | NEUROG3 |
| TOMM7 | EIF2B1 | RSL24D1 | RANBP3 | PTGFR | Rpl3 |
| XLOC_I2_009328 | MKNK1 | TECTB | FAM104A | NR2C2 | C19orf25 |
| PPP1R3C | TRAPPC13 | IL20RB | CLEC4A | ANKRD16 | MIR99AHG |
| KCNK6 | GAD1 | LEAP2 | MXD4 | DCK | TPH1 |
| C5AR2 | BUB1 | NBPF6 | SELENOS | DLST | PLA2G1B |
| PDGFRB | SMYD3 | ANK1 | CCPG1 | TMEM120A | ZNF181 |
| RFTN2 | SNRNP70 | SPIN2A | EFEMP1 | CLIC1 | CHRNA7 |
| LINC00898 | CYBRD1 | USP49 | NUCB1 | TCTN3 | SPACA4 |
| XLOC_I2_007488 | OR2T33 | SMOC1 | CEACAM21 | MKNK2 | UHMK1 |
| PDE6H | SLC35G2 | TMEM80 | GBP3 | EFNB2 | KIAA0391 |
| PNCK | ADAM7 | C14orf1 | ZNF763 | OXCT2 | PHLDA2 |
| PODXL | XLOC_005712 | FAHD1 | MMP10 | SAMD4A | PAAF1 |
| DYNC1I1 | NPY | XCL1 | FKBP7 | MRPL49 | CRMP1 |
| GPR55 | DDX60 | TNFAIP8L2 | SYT14 | RPL30 | NDUFS5 |
| C1orf220 | OR6F1 | C7orf26 | SLC25A2 | TAF1C | MCOLN1 |
| IL22RA2 | LDOC1 | ZNF488 | DUSP7 | RSL1D1 | GNL1 |
| MCEE | GAS2 | CRYZ | ZNF706 | PTPRM | ASPHD1 |
| EQTN | CAMK2B | PTBP1 | HAUS3 | CH25H | EIF1AD |
| FRMD6 | NDUFB10 | WISP3 | CD276 | Bnip3 | EPB42 |
| DPH3 | HIST1H3C | C9orf16 | MAP3K7 | DIEXF | ERN1 |
| P2RX7 | PURB | XLOC_005466 | ZNF624 | CAT | ARPC3 |
| DDX1 | HSPA8 | LOC107987587 | KLHL1 | CXCL3 | AGTR1 |
| KIR2DS2 | CYP4F12 | TARP | TSR3 | THAP10 | FAAP24 |
| GPR180 | UBALD1 | SMIM11A | CHPF | TUBGCP4 | HLA-DRA |
| NPHS2 | MRPL43 | ABL2 | ACOT6 | PAIP1 | NF2 |
| RAMP2 | CCDC167 | BEND7 | LIPT1 | C3orf18 | TPK1 |
| KRTAP13-4 | PDP1 | TXNRD3NB | NEK7 | ELAVL2 | GIF |
| DLG3 | Nrbp2 | CDS1 | FA2H | VPS39 | SPATA6L |
| EPS8L1 | CRY1 | MKRN3 | S100P | XLOC_I2_013931 | SPHK1 |
| CKMT2 | CNNM3 | H1FOO | NPPB | SOX12 | MVK |
| CTDSP1 | CCDC51 | INSR | ZNF100 | CD96 | CCL4L1 |
| LOC390877 | HORMAD1 | PFDN2 | Pi4ka | RUVBL2 | P4HA2 |
| KLK15 | CTNNA1 | LENEP | SGO1 | EFCAB14 | ABCG2 |
| BAMBI | NFKBIA | ERICH2 | NT5DC3 | TRIM48 | SNHG11 |
| RBM26 | AK5 | LOC650293 | STXBP4 | CD46 | AHNAK |
| NUP50 | MRPL35 | RBP2 | GSK3A | NCAPG | ZBTB44 |
| PSMF1 | LYAR | FAM65B | KIAA0408 | CCR1 | SLC25A18 |
| USP7 | TXLNA | MIS18A | LBP | MAPK15 | LOC613266 |
| CDKL1 | PPTC7 | CATSPER4 | FAM120C | PRAMEF5 | OAZ3 |
| DOCK4 | UQCR10 | POP4 | TMEM234 | C11orf84 | EXOC3-AS1 |
| ANXA5 | SPCS2 | ADPRHL2 | ZNF776 | PRKAR2A | ADGRG3 |
| PCP4 | MRI1 | PPIH | HIST1H3B | CEACAM21 | STARD7-AS1 |
| IFNA4 | CGB8 | OLFM1 | CRCT1 | INGX | STK33 |
| NUTF2 | CPT1B | HSPBAP1 | TSPAN8 | TNFRSF13B | MND1 |
| TMEM208 | SRPRA | ARSG | GAGE1 | PIM3 | RPN1 |
| SMIM7 | TNP1 | RNF7 | PRPF4B | TAAR8 | TMEM26 |
| CYSRT1 | RBP5 | IGFBP2 | ZNF189 | SCG5 | C19orf54 |
| ALG9 | KRAS | C20orf85 | PPARA | AKR1B1 | VPS37A |
| TSG101 | LARP4 | F2RL2 | GPR160 | ACOT11 | NARS2 |
| PLAU | GLYAT | PDPN | NIPAL3 | NRBP1 | CAPN13 |
| CSNK1G2 | NDUFA11 | GALNT3 | GPR37L1 | CAPS | PTGFR |
| CDCA8 | MRPL53 | TRIM51 | MRPL4 | CTBP1-AS2 | MAPK7 |
| EIF4EBP2 | PGK2 | TWF1 | TMSB4X | RSPH1 | STK3 |
| MTAP | FAM45A | HBP1 | TTC29 | TMEM82 | FFAR2 |
| ACAD11 | LELP1 | CCND3 | KRT6A | MYCL | PYY |
| TBL1Y | XLOC_008618 | ZNF438 | TMEM154 | RIBC1 | STAT1 |
| LINC00602 | LYRM7 | SNF8 | RACK1 | CLK3 | CARNMT1 |
| C5AR2 | Emilin1 | TRIAP1 | NSG1 | TAS2R41 | HDAC6 |
| DAP | B4GALNT2 | PPP1R1C | MAP3K3 | CLPS | IP6K2 |
| LTF | PWP1 | TBC1D7 | CRABP2 | DIRAS2 | LOC148413 |
| DSCR10 | APCDD1 | FAM221A | KCNAB1 | PDLIM3 | ZMAT4 |
| LIMK1 | LINC00638 | XLOC_I2_008131 | HCRTR1 | ADAD1 | TYW5 |
| TCEAL8 | HAT1 | TAS2R1 | SNAPC1 | LOC102724229 | HNRNPUL1 |
| CAMK4 | AGTRAP | PFDN5 | EEF1A1 | CPA2 | TMEM47 |
| WDFY3 | PAPSS2 | IL17RC | RUVBL1 | MSRB1 | NEK11 |
| ASB13 | PI4KB | FAM228A | FMO5 | LXN | SHMT1 |
| PPIG | PCDHAC1 | HAO2 | PSMF1 | DIAPH3 | ALG1 |
| PTPRS | ZFYVE21 | C17orf67 | INPP5A | NAGK | PAK5 |
| R3HDM2 | PLA2G5 | TKFC | C5AR1 | GK5 | S100A10 |
| CSNK2A1 | KIAA1586 | HAUS4 | TNFAIP1 | Ergic2 | HAX1 |
| NECAP2 | SCLT1 | SLC30A8 | FAM189B | IP6K2 | SERPINC1 |
| CKMT1A | OAT | PRDM5 | TET2 | AURKC | STX2 |
| GRM8 | PXK | LILRA4 | MPP3 | FBXL14 | GNPDA1 |
| HSD17B1 | RPN2 | MRPS33 | MGAT4C | RPS27A | AK5 |
| CLEC18C | PTPRN | S100A6 | LIPT1 | ZBTB37 | FGF8 |
| IMMP1L | ATP6AP2 | MPPED1 | AQP4-AS1 | MRPL27 | DUSP23 |
| POMP | SCML4 | VENTX | SCD5 | SP3 | DPCR1 |
| PIKFYVE | SAMD12 | WFDC11 | LOC107984064 | ISOC1 | POLE2 |
| RWDD4 | TMEM64 | SULT4A1 | DDX19A | PKD2L1 | SLAMF7 |
| SLC30A2 | BNIP1 | DEFA1 | HIST2H2AA3 | RPS7 | FAM64A |
| WBP2NL | PSMA2 | BCS1L | SUGCT | HCAR1 | IFT20 |
| XAGE2 | CHRM3 | ZNF776 | SNCA | CLIP4 | GPX8 |
| CDH19 | DDX31 | DMWD | IL23A | PITPNC1 | CMPK1 |
| GXYLT1 | GSDMC | TMUB2 | CNDP1 | SPEF1 | SPINK6 |
| TMEM237 | KARS | VEGFB | CCDC70 | LOC653513 | SLC25A47 |
| ASCL2 | RBM45 | PFDN4 | UGGT2 | GALR1 | Mon1a |
| ETFB | HNRNPR | RAB32 | RHOC | ENPP1 | GPR39 |
| COQ8B | MGC70870 | GPSM3 | NUTM2F | PSMB8 | EGFR |
| OR6C4 | BTK | PHACTR4 | C3orf56 | PDP2 | BAALC |
| ZNF354A | TMEM91 | PAK4 | DAPL1 | TRHR | OARD1 |
| NUMB | Hdac7 | UGT1A6 | POMK | CCDC113 | INS |
| HILPDA | BIK | CAP1 | PNKP | NAT16 | GINS3 |
| CHORDC1 | PPP1R36 | SNX3 | COX17 | DNAJB3 | KCNE2 |
| BCL2L12 | XLOC_I2_013192 | KPNA1 | TMEM205 | HPSE2 | FGFR3 |
| CDYL | RUBCNL | HK1 | FCER1G | LEF1 | CD248 |
| PAQR6 | FAM173B | RPL39L | DNAH14 | RNGTT | HMGCLL1 |
| SRP9 | EPB41L4A-AS2 | FAM122C | EVI2B | LPAR1 | CDY2A |
| SUCLA2 | RXFP3 | PCDHB11 | EPHB2 | MC3R | PITPNB |
| UBE2L6 | TGS1 | KRT18 | CCL13 | FLYWCH2 | DUSP16 |
| DHRS2 | TSPAN16 | TRIP10 | CST9L | ZNF829 | CCDC68 |
| NME5 | LRG1 | LGI3 | LOC100287896 | USP36 | CASP6 |
| KCNK16 | VEGFA | TM2D2 | THYN1 | SQSTM1 | DNM3 |
| CCDC60 | LOC644936 | SZRD1 | PRSS21 | SGK2 | CPM |
| OR6N1 | C11orf74 | TNF | P2RY6 | RPL22 | SLC25A51 |
| TAS2R10 | CCT6B | ZDHHC15 | ZNF124 | BCKDK | VIP |
| LRRC45 | LPAR3 | MDH1 | FMR1 | HMOX1 | TROAP |
| RPS12 | REPS1 | TYSND1 | RRM2 | XLOC_013491 | MBL2 |
| SPOCK3 | FGF11 | AXL | TTLL10 | GMCL1 | PCED1B |
| RPL18A | AVP | MBIP | DPY19L4 | COPS8 | LINC01547 |
| GKN1 | EDNRA | SLC12A8 | VNN2 | KLK7 | RGL4 |
| SERF1A | CCNE2 | ERP44 | ATP6V1C1 | RPLP0 | GPR1 |
| GNG7 | SCAND2P | PPME1 | SURF1 | TMSB10 | STOX1 |
| CEACAM1 | BRK1 | SMUG1 | MRPL15 | LYPD5 | MSR1 |
| CCNH | PCBP4 | COMT | WFDC10A | RPS6KA5 | TRIB3 |
| FGR | LRRC25 | KLK1 | SPATA8 | TFPT | MKRN2 |
| SYT17 | E4F1 | ATRAID | GCGR | SIAH1 | GPR19 |
| THRAP3 | PSKH1 | CELA3B | CNIH3 | PLGRKT | GPR132 |
| CCM2L | FASTKD1 | NT5C1A | KIAA0368 | FES | ODF2 |
| CYCS | GBA2 | LHFP | PCDH12 | NELL2 | ACAD9 |
| NAGK | PI4KA | APOC2 | TMX1 | HDC | PRKCE |
| TECRL | ASTE1 | CD22 | LBH | DHRS7 | RHOG |
| MSRB3 | SKA1 | C9orf72 | FAAP24 | STARD5 | SPHK1 |
| BEND5 | SELENOW | DHX29 | SLC14A1 | ZNF398 | SSBP3 |
| MRPS11 | ATP5C1 | RAMP3 | C1QTNF2 | PDCD2L | CATSPER2 |
| LOC102724993 | PDLIM2 | ATP5G3 | IFRD2 | OR4C6 | FBXO3 |
| RAB5A | CACNB1 | IL5RA | ASAH2 | IFT46 | SRPK3 |
| QRFPR | PAX4 | MAPT | TRAPPC6B | MGAT1 | DRG2 |
| GPR50 | EEF1D | AP2S1 | EPB41L3 | CABP7 | PTMA |
| CCDC58 | CCL25 | FBXO38 | IGFBP4 | TSLP | STK38 |
| CDKN2AIPNL | D2HGDH | ASIP | FBXO10 | OR2G3 | TBPL1 |
| C16orf59 | OR5I1 | PIM1 | PFN1 | KCNA10 | PLEKHA7 |
| TUBE1 | YTHDF2 | ZNF571 | AIFM1 | PSKH1 | SAP130 |
| IFNL1 | TMEM208 | NOP2 | TAF5L | HIST1H2BF | PRSS48 |
| TAAR8 | TNFAIP3 | SYPL1 | OPN1SW | FMO2 | REM1 |
| BSCL2 | DND1 | PAQR3 | SCIMP | PIK3C3 | TSR1 |
| GRTP1 | TAGLN2 | C1QTNF1 | CDK3 | STAR | SMG1 |
| LGI4 | PDGFRA | SLC25A4 | SF3A3 | BORCS8-MEF2B | CYP2A6 |
| CASP5 | NRG1 | LRWD1 | MNAT1 | MAGEA4 | ANGEL1 |
| TAX1BP3 | DDX47 | INS-IGF2 | PAK1 | N4BP2L1 | WDR86 |
| RCAN1 | PAK1 | PHF20L1 | XG | ACP6 | SLC28A1 |
| TK2 | ASNS | CLEC7A | LCK | ZNF778 | METTL8 |
| ATP6V1G2 | RIPK2 | FCGR3B | ANXA13 | NDUFB1 | ASAH1 |
| DTWD2 | RLIM | PLIN3 | TUBB3 | TRIM31 | HCAR3 |
| SOAT1 | XLOC_012222 | WBP2NL | COMMD1 | SYT4 | PEX19 |
| CHRND | CALHM3 | ANP32A-IT1 | USP38 | EIF4A2 | MTMR9 |
| TFPT | KRT83 | WDR47 | UBE2K | SH2D1B | TNFRSF9 |
| GPR50 | TMEM159 | ATG14 | CSF1R | LSM10 | WNT3A |
| WDR4 | SPINT1 | H1FX-AS1 | TAF15 | PCDHA10 | LRFN4 |
| ZNF549 | STK40 | LINC01270 | DYNC1U1 | IDNK | DNAJB7 |
| HOMER1 | SLC39A12 | MYOCD | AK2 | TANK | MED22 |
| SLC35B4 | TESK1 | ATF2 | PRKCSH | SENP8 | S1PR5 |
| CELA3B | MGST2 | LPAR4 | NR0B2 | GPM6B | FAM131C |
| RASL12 | ZNF669 | TINF2 | KLHL29 | RAF1 | SCGB1A1 |
| ACBD4 | ST8SIA4 | IRF2BP1 | CHPF2 | FOXJ1 | MRTO4 |
| NUMB | RNF167 | GABRA6 | ALDH18A1 | PODNL1 | C20orf96 |
| TAS2R40 | GPRIN3 | SLC25A41 | CRIM1 | NCMAP | ADRB2 |
| DDX20 | SCYL2 | POLR2M | CCL23 | FDFT1 | SCYL1 |
| EMC3-AS1 | ORC4 | CABYR | WNT4 | NIFK | NREP |
| SCO1 | ACYP1 | TEX37 | CPEB2 | ERO1A | STK40 |
| MRPS30 | LIG1 | RNASE3 | STK38 | PRND | CCKBR |
| TMC5 | OR6V1 | CADM1 | ZC3H7A | TAZ | HAUS2 |
| TNFRSF19 | ANXA4 | CLCA2 | GATS | GJB2 | CHRM3 |
| EFS | ENC1 | PLEKHH2 | SPATA6 | IL12A | FKBP8 |
| ENOPH1 | CCT8L2 | TIGD7 | EIF4E3 | S100A11 | ZNF436 |
| FBL | POLR3F | SUSD4 | ZNF483 | EEF1G | MTUS1 |
| CD28 | NRP2 | GDPD3 | CSNK1G1 | DNAJB1 | IST1 |
| RFWD3 | ACTR8 | CDH6 | SPRR2A | STMN1 | CD9 |
| SLC7A1 | LOX | Ola1 | ASMTL-AS1 | CIART | RGR |
| PLAC8 | UHMK1 | MAPT | DTX4 | ZNF419 | SLC35A2 |
| CEP126 | RARG | ZIC1 | GLRX2 | EIF2AK4 | STX18 |
| DIMT1 | GUCY1A3 | PAPLN | SLC36A3 | XLOC_013119 | TRMT12 |
| XCR1 | FABP12 | BOLA2 | KIAA0930 | NAT8 | SPACA3 |
| CALM1 | XLOC_I2_008203 | SLC51B | WDR92 | CCNE2 | ATPIF1 |
| GHRH | CAMKK1 | XLOC_013643 | ABO | VPS26B | KCNE3 |
| PQBP1 | LINGO2 | HPS1 | YPEL1 | PCDHGB3 | APOH |
| ARF6 | KIAA0040 | PTCHD3 | WRAP73 | TMEM211 | RPL23AP64 |
| LTF | MTNR1A | MYOG | SHISA4 | KIT | RAB39B |
| FAM172A | CETN2 | GJA5 | GPN2 | NCK2 | OIP5 |
| PRKG1 | OSCP1 | RHOBTB1 | PIK3R3 | TSEN15 | HTR6 |
| TSSC4 | BACH1 | CRBN | HM13 | CAPZB | PRKAG2 |
| C20orf24 | EBF1 | SBNO1 | ARHGEF7 | CASP2 | TTC16 |
| SETD7 | MAPRE3 | TRIP6 | BTN2A1 | SLC6A15 | ZCCHC7 |
| PPAN | RTN1 | KIAA0087 | RBKS | BPGM | HVCN1 |
| CA12 | MELTF | M1AP | FAM60A | LHB | SRPK3 |
| PRDM14 | PTCD1 | NRM | NFIA | TEFM | TALDO1 |
| FAM46A | SULF2 | SYT11 | NR1D1 | POLR3C | SERPINA4 |
| ZNF695 | KRTAP3-3 | PLSCR1 | NT5C1B | DYNLRB1 | STK35 |
| BMP7 | PSPC1 | DPEP2 | IFNA21 | SRP9 | MRPS36 |
| AGL | CSNK2B | SLC31A1 | CCNYL1 | CSNK1A1L | KRT8 |
| ASF1A | MOBP | QARS | TEKT2 | GADD45B | ELK4 |
| VWA5A | TSPYL5 | DAGLB | TRIM16 | LRFN4 | ELANE |
| OXNAD1 | EPHX1 | AK2 | ZIM2 | H2AFV | PTH1R |
| MB21D2 | STX3 | NEK8 | EDA | C1orf27 | VAPA |
| UNKL | MSS51 | ISLR2 | STK26 | ADGRG5 | PAQR7 |
| ZNF501 | MCRIP2 | CAMK2A | CNST | TSSC4 | MSH4 |
| SLC35E2B | C4orf3 | NME8 | FAM207A | LINC00238 | OR14C36 |
| MAX | ENSA | MT1A | OPCML | RASSF5 | PRKD3 |
| TMEM8B | DOCK8 | AKR1C2 | RPS19 | TMEM106A | FAM212B |
| CYFIP1 | TMEM9B | ZSCAN20 | C1QTNF9B-AS1 | TEX13A | TECTB |
| AURKB | ADD1 | AKR7A3 | NIT1 | SERPINB10 | SLC27A6 |
| PIM2 | PIM1 | ASPH | SPINT2 | ADAP1 | ACYP1 |
| CPXM2 | BMI1 | CDC26 | YWHAQ | RGS17 | TP53RK |
| TBC1D19 | ADRB2 | COX6A1 | CASC2 | SMR3A | TSPAN5 |
| DBF4 | HCFC1R1 | ZNF281 | C4orf36 | SRMS | SHD |
| ORMDL1 | DAND5 | GPM6A | BEND5 | ZBTB10 | EXOC4 |
| AKAP14 | ZRANB2 | C20orf173 | GALNT4 | FTL | FGF6 |
| SGK1 | KBTBD3 | GORASP1 | ZNF285 | TATDN1 | FAM90A1 |
| GPR84 | PRMT8 | CKMT1B | H2BFWT | HK2 | ZNF468 |
| C2orf40 | OPTC | FAM166A | HIST1H3F | FAM189B | PYCRL |
| PWP1 | RSPH3 | PKN2 | PEX7 | PRUNE2 | CPA3 |
| FBXO7 | TIAM1 | FAM222A | ITM2B | C9orf163 | LSP1P3 |
| COQ10B | ZNF792 | ACTRT3 | RAP2C | MAL | IFNA10 |
| CCDC27 | CSMD2 | RFXANK | ING1 | CD40 | THOC3 |
| KRT18 | PSMB4 | ESRRG | EWSR1 | DCTN5 | ZNF295-AS1 |
| C12orf57 | DCAF5 | DKKL1 | FAM118B | CACNG2 | ALDH1A3 |
| Myo5c | KCNS2 | SMIM14 | ATG9A | EGFR | LINC00312 |
| MYH7B | TP53TG3 | WWP2 | PRKACB | LOC102724428 | DEUP1 |
| SOX14 | MPP1 | FGFRL1 | ASRGL1 | NUBPL | CCDC117 |
| SCPEP1 | MAP2K3 | AK4 | MTMR14 | ARSE | MAGEB3 |
| MAPK10 | MAP3K5 | TSSC4 | RNF167 | FEM1C | IMPA1 |
| PLD3 | WASHC1 | NME4 | GJA4 | PTCHI | PIM1 |
| PIM2 | G6PC2 | FBXL13 | GP1BA | KLHDC3 | MRPL9 |
| KRT6B | ASB2 | FGFR4 | JAM2 | REG1B | XLOC_I2_009889 |
| ROM1 | KLHDC3 | MAATS1 | MITF | DAW1 | ARHGAP25 |
| DNASE2 | ERBB2 | CDKAL1 | Adamtsl1 | C2orf83 | CCL18 |
| CNBD2 | NR2E3 | STRADA | PPIA | UBAC2 | CXCR5 |
| COPS5 | BCDIN3D | DCAF7 | C19orf66 | IRF4 | GALK2 |
| NDNF | ELSPBP1 | SMCO3 | PDK1 | ALPI | CXCR4 |
| STAG2 | KRT40 | GATC | GABRB2 | RDH12 | TSHR |
| CXCL16 | MEMO1 | PRPSAP1 | DUSP14 | ZCCHC17 | ANXA2 |
| IGLL5 | P3H1 | GCC2-AS1 | GNG10 | RAB37 | GRP |
| TAS2R16 | TRIM31 | CDK7 | IWS1 | TKT | ACTR10 |
| FAM89B | WBP2 | ILKAP | TMEM54 | OPN4 | EEF2KMT |
| ZNF768 | TICAM2 | NUDT10 | VN1R4 | TBCCD1 | AGTPBP1 |
| PTHLH | NSUN5 | ATP2B2 | B4GALT5 | TMBIM1 | SERPINB9 |
| C4orf26 | RASSF1 | ISM2 | SPPL2A | GSTZ1 | C4orf19 |
| OR10K1 | PARL | GRK5 | SMOC1 | JKAMP | GUCA1C |
| CST5 | PRKCZ | C2orf73 | DEPDC7 | GSTT2B | LRIT3 |
| PRR14L | SELENOT | SPINK4 | BLM | PRELID3A | ERCC8 |
| HOMER1 | VANGL2 | AGMO | CALD1 | METTL6 | PSKH1 |
| TMEM43 | LINC01341 | NUDT11 | CTH | ZSCAN26 | KCNAB2 |
| LOC90768 | CCL21 | CLDN8 | FTO | CLC | MAPK1IP1L |
| PCDH10 | CHN2 | STK4 | FAM83F | MED7 | CRABP1 |
| SPPL2C | FAP | SBDS | C10orf91 | CALHM1 | HIST3H2BB |
| TMTC1 | C15orf54 | UBE2W | GABRR1 | ART3 | FASTK |
| NEK9 | SPATA17 | LETM2 | CFAP52 | OXLD1 | CAMK2G |
| MMADHC | HLA-C | TTI2 | ETFRF1 | HEXB | MRGBP |
| 42981 | AP5M1 | SGK3 | C8orf49 | LSMEM2 | ZNF322 |
| PRTN3 | NTMT1 | MYO19 | TMEM218 | LGALSL | WDR33 |
| PTPN20 | USP16 | ICOSLG | MIXL1 | ANKRD20A4 | CACNG4 |
| SH3BGRL3 | EIF3D | COMMD6 | ST6GALNAC4 | YIF1A | MUCL1 |
| MRPL21 | FGFR2 | COASY | OR8H2 | XLOC_I2_014086 | SNRPE |
| LOC107987235 | TGFB2 | OR10X1 | TDGF1 | XLOC_I2_009790 | EIF5A2 |
| LTK | VASP | GORASP1 | GPR119 | LAPTM4A | GTSF1 |
| GPR151 | KRTAP4-1 | SCIMP | GLRX | ALPK1 | AKT2 |
| PRSS2 | SPATA18 | UCP2 | TMEM246 | GPR61 | DUSP22 |
| CIDEB | NKAP | PIM3 | CD226 | ARL17A | STX8 |
| SHISA3 | CD86 | PKM | MLLT11 | ADM | PPP6C |
| TREML2 | FYN | XAF1 | GLT8D2 | DCAF12 | OLR1 |
| CYP2A7 | CENPA | NEMP1 | EMG1 | LDLRAD4 | RAB4A |
| CCDC26 | TTC25 | CCNL2 | FAM227B | PURG | ZNF496 |
| NMBR | SERPINF1 | TMEM259 | CDK2 | IL23A | CDK10 |
| KPNA2 | APCS | COMMD8 | XLOC_I2_000581 | RNF216 | TP53 |
| AIPL1 | JAK1 | GOSR1 | SDSL | SDS | SLC25A35 |
| FRK | GLMP | PSMD2 | MAGEA10 | PGC | CD300LB |
| CHRNB1 | CD302 | AP3M1 | OMP | CBWD1 | RAB30 |
| HMGB4 | CASC10 | AGPAT2 | PGAM2 | LSM2 | PLPP7 |
| MTUS2 | TMEM242 | GPX4 | PSMG1 | NUBP1 | CYP2F1 |
| PDE6G | OR10X1 | ERLEC1 | ANPEP | KAT2A | PDDC1 |
| FANCI | OXCT2 | ABCB9 | OR2T10 | IFNA8 | TMEM196 |
| LIN9 | LY6H | APOPT1 | NARF | ZNF24 | CRACR2B |
| ULBP1 | LSM1 | PSTK | NLK | ATP5J | ERICH6 |
| MRPL34 | ATP6V0A4 | NACC1 | TMCO6 | MFSD10 | CYSLTR1 |
| NGLY1 | OR56B1 | IRAK1 | CMPK1 | PAK4 | ADGRD1 |
| RDM1 | TMEM43 | SSTR2 | OLFM3 | SLC50A1 | KIAA1644 |
| MAP4K3 | C5orf64 | PSENEN | TMEM184B | PHTF2 | TRNT1 |
| RNF11 | CCDC88C | PIK3CA | UBA52 | GLRA3 | BAG1 |
| ST3GAL5 | TBC1D29 | BCL2L12 | XLOC_I2_005179 | GPR139 | PKD2 |
| PAQR7 | DPY30 | TTC26 | FAM26E | FRZB | SYT1 |
| TNNT2 | WDR31 | TUBB3 | UBR7 | ANAPC10 | RNF130 |
| SFXN5 | NME7 | MTUS1 | DEPDC1 | LTB4R | CNN1 |
| THNSL1 | NR2C2 | LIM2 | DIP2A | C20orf203 | SC5D |
| CHMP5 | CRK | MICU2 | EPN2 | GAL | DEFB123 |
| CXCR3 | SERPINA6 | MAPK12 | MIF | FAM166B | KLRC1 |
| PROKR1 | GBA | MFAP4 | LSP1 | XLOC_I2_015578 | STARD3 |
| KDELC2 | KLK1 | IMP3 | FDPS | RC3H2 | STPG1 |
| JRK | ROPN1B | XLOC_I2_007835 | CXCL9 | PMS1 | BPIFC |
| FUNDC2 | OCIAD1 | C14orf159 | TUBB6 | DHRS2 | DEFB127 |
| DRD2 | TMEM268 | MAPKAPK3 | ECHDC3 | CRYM | PSMC4 |
| ANKS6 | PTGDR2 | MAP2K3 | ENKUR | MAP3K19 | LAMC1 |
| CD300C | CNKSR3 | XLOC_I2_009492 | SMPDL3B | KRTAP13-3 | COLQ |
| VCY | RTP4 | C3orf35 | GDF15 | CD8B | WNT2 |
| LOC101927905 | RHOXF2 | MRGPRD | MBOAT2 | RNF151 | ACTR3 |
| ESRP1 | NRG4 | GPR32 | CHRNA4 | LRRN4CL | LATS2 |
| CPNE8 | RABL2A | TSPO2 | C10orf53 | PPIL3 | MAP3K7 |
| ZWILCH | CELA3A | CDK5R1 | FUOM | AKR1B10 | GMPPB |
| LSM5 | ICK | NEDD8 | FCGR3B | AATF | LOC653513 |
| ING5 | SH3GL2 | KCNIP4 | ZNF784 | UCHL5 | LRTOMT |
| ABCB1 | PML | MAGED1 | F2R | LTA | KLHL28 |
| SNX10 | CRP | HMGB3 | SLPI | ALDOC | UHMK1 |
| KDM4B | H1FNT | CDK7 | TNFSF14 | PSMD6 | LHFPL5 |
| RHBG | YIPF2 | TBC1D13 | DDR1 | RGR | RAB25 |
| WRNIP1 | TPRN | ASZ1 | FCAR | VIPR2 | TRIM73 |
| GABARAPL1 | CDC16 | APOE | CAMK2N2 | PLD5 | MPLKIP |
| RGS13 | PLA2G1B | SRSF10 | FDXACB1 | EIF5A | C7orf13 |
| RYBP | ACTR6 | MCFD2 | TNFRSF21 | CIB2 | TLE1 |
| STAP2 | WDR77 | NAA38 | Hoxa10 | IL20RB | CPE |
| MAEL | ACP1 | POLE4 | CCDC28B | DNAJC2 | DGCR14 |
| SULT2B1 | MAPRE1 | DNAJC22 | VPS28 | IRAK4 | SLC18A3 |
| CDCA4 | NDST1 | XLOC_I2_006804 | SSR3 | ING5 | FMN1 |
| MYL6 | PDIK1L | PLSCR4 | CARD8 | PTGER4 | ZFP14 |
| SPX | CDK2 | GRID1 | TPX2 | TMEM52B | NMUR1 |
| KLHL33 | HS3ST3A1 | TAS2R20 | ERBB2 | CCDC148 | KCNE4 |
| AARD | EIF1AX | NPM1 | TESK2 | DUSP23 | PRR15 |
| CSTF3 | NUDT5 | GMDS | HMBOX1 | TMEM74 | HLA-DQA1 |
| MPC1 | SPIN2B | TULP1 | MAPK10 | ALDOB | HPN |
| CSNK1A1 | C18orf54 | BTF3L4 | HRAS | STX7 | H1FNT |
| GPR26 | RDH12 | GNAT2 | XLOC_013923 | XLOC_002741 | CIB3 |
| GNPTAB | YWHAEP7 | SSMEM1 | NUDT12 | ZNF705D | CACFD1 |
| MLF2 | NUSAP1 | TMEM53 | KRTAP19-4 | PDK3 | HCK |
| MRPS14 | CCDC25 | STEAP4 | EHD4 | B3GNT6 | RPRD1A |
| GRAMD1B | SNHG7 | RPL23A | ANKRD44 | SIRT4 | ENTPD7 |
| CD36 | TOX | PSORS1C1 | ABT1 | GALNT16 | RPS2 |
| PDK4 | RNFT1 | ARL17A | CNR2 | TPT1 | TRAPPC2 |
| EFCAB12 | CORT | NMUR1 | FAM175A | PPIL6 | FDPS |
| TTLL2 | IL1RL2 | NUP62 | PEX5 | LINC00671 | LEP |
| SLC25A43 | DEFB118 | NPY2R | GBP5 | PIM2 | B3GNT9 |
| STAMBP | PROSER3 | RFPL4B | RALYL | MYOZ2 | UPRT |
| PPP1R26 | SUV39H2 | XLOC_I2_004840 | OGFOD3 | NRXN3 | LOC440461 |
| SRD5A1 | ITLN2 | RPL23A | CHCHD4 | APOC1 | XLOC_004269 |
| SKP2 | TNFSF9 | XLOC_013901 | UBE2W | IKBKB | LINS1 |
| IFNA13 | SH3RF2 | MRGPRE | CCKAR | SETD3 | NRIP2 |
| TPK1 | IL23R | PLSCR1 | KIR3DX1 | CIRBP | SH2D4A |
| CHRNA7 | SATB2-AS1 | GLIPR1 | MCAT | GTPBP10 | DNAJB2 |
| STK26 | MAFG | SMLR1 | MCM5 | PRKAR2A | TIPRL |
| RAD51AP1 | PITX1 | HAND1 | RPIA | TACR3 | ACP1 |
| OR14I1 | FAIM | EPHB3 | CNTN4 | GPR157 | ITGB8 |
| LINC01580 | PTGES2 | ADAT2 | DEFB119 | ING3 | RASL11A |
| GRK4 | PTPRCAP | C6orf25 | FZD1 | HIST1H4J | HNRNPC |
| PLSCR5 | HNRNPH2 | KCNJ11 | NTSR1 | GPN1 | OPCML |
| ATP6AP2 | LOC100101148 | CDRT15 | KRT36 | PLCG2 | NDUFA8 |
| CLMP | OR5H6 | MT1M | MAP3K20 | QKI | HIST1H2AG |
| NTSR2 | PFKM | FMO4 | SH2D1A | TMEM120A | HTR4 |
| FAM107B | GNG7 | OR8G5 | GPR146 | ATAD1 | ANKRD13A |
| HMGN4 | BDH1 | UBXN2B | FGF22 | THUMPD3 | SLC25A10 |
| TESK1 | MAPT | FRG1 | SLC35E2 | GNG8 | DUSP22 |
| XRCC4 | IP6K1 | C3orf33 | FXYD2 | FXYD1 | SMCO1 |
| SNX24 | MDK | NOP9 | PLEKHF1 | KDELR1 | TSNAX |
| RPL6 | HOOK3 | DQX1 | PSMD2 | CLIC5 | MAD2L1 |
| HSF2 | VSIG1 | CAV3 | CHST6 | LMBRD2 | WDR90 |
| GHSR | SYNC | CDK5 | TCTN3 | GPX7 | MAP3K9 |
| MBNL1 | CDK17 | GGTLC1 | GPN3 | SIGMAR1 | SC5D |
| RCBTB1 | HMGA1 | HOOK2 | OR11H4 | TRIB3 | UBE2V1 |
| SUSD3 | CHRM2 | HMGN2 | OTUB2 | CCL2 | AURKA |
| LIMS3 | MGARP | REEP3 | CYB561A3 | SLC10A7 | FAM109B |
| PEF1 | CLUAP1 | KYAT1 | CTRL | FAM127A | RBM7 |
| FFAR1 | CD244 | MAS1 | CTRC | HMGCL | SDF2 |
| PTGIR | PCMTD2 | COL20A1 | GPR34 | PTPA | GPR63 |
| CD3G | PTAFR | CTSK | SPCS1 | HIST2H2AA3 | XPR1 |
| MOK | NMT2 | XLOC_I2_009281 | NXPH2 | GFM2 | TRAF4 |
| RALY | PPP1R32 | PPP1R3B | SSBP3 | IFT74 | TMSB10 |
| RRM2B | ACPP | EIF3E | CBFB | RXFP2 | AKR1B10 |
| Tex261 | GFRA2 | CPD | EDDM3A | AK1 | SAV1 |
| FOXD4 | TM6SF1 | ASNA1 | SLC7A11 | DEFB105A | ARHGEF26 |
| EIF3G | PRH2 | CSF2 | APOOL | BPHL | ARL13A |
| RSL24D1 | MAP4K4 | MRPL43 | ARHGEF3 | UCK2 | STATH |
| ARL6IP6 | MAP6 | TPT1 | SPHK1 | RCSD1 | C4orf33 |
| SUPT4H1 | KCTD17 | MAN2B1 | PEX10 | NCCRP1 | XLOC_005602 |
| SLC25A6 | PMCH | B3GAT2 | ZFAND6 | DDIT3 | PPARG |
| MED20 | SLC29A1 | LOC107983965 | DYNLT3 | LOC439933 | TOE1 |
| TCEAL1 | SCD5 | ZNF671 | SPHK1 | GPR182 | CHTF8 |
| NUDT1 | B3GALNT1 | RPP30 | NUDT3 | MAPKAPK2 | SCGB1C2 |
| ABI3 | PCGF6 | GLRA2 | GYPB | CRTC3 | EMCN |
| CCSAP | PRR19 | FAM83A | XLOC_I2_006131 | TNFSF9 | ATG7 |
| PVR | SELENOF | CXCL13 | TMEM256 | ZNF597 | EFCAB2 |
| S100A16 | THPO | OTUD6B | MRGPRF | XLOC_010651 | ZNF75A |
| SRSF2 | PCCB | SLC22A15 | PELI1 | EGFR | NIPA1 |
| TMEM126B | ZDHHC20 | SYNE3 | NAT2 | AQP3 | ATPAF2 |
| ARHGEF9 | TESK1 | QRFPR | CPT1A | RND3 | IRAK1BP1 |
| GALNT15 | EPM2AIP1 | TOP3B | PDXK | TPD52L3 | RAPSN |
| PAK1 | HLA-L | GPR143 | TSHR | AGK | CX3CL1 |
| CYSLTR1 | C8orf31 | NHSL2 | LMNB1 | C10orf35 | C5AR1 |
| FAM199X | POU2AF1 | TP53TG5 | CAPSL | MMP28 | GOSR2 |
| HIST1H2AC | CXCR3 | SPRY4 | NO_MATCH_203 | IL21 | ZNF662 |
| ZDHHC5 | FADS3 | NO_MATCH_181 | NEK6 | TOMM20 | NCR3 |
| UCN2 | TMA16 | GCDH | BCL2L13 | CSNK1G1 | METTL18 |
| RAMP1 | STK33 | BRAT1 | BAK1 | CMTM8 | OR1D2 |
| HDHD3 | EGR1 | SPRYD7 | ABHD17B | NPM3 | ASTN2 |
| NO_MATCH_25 | TWF2 | TNFAIP8 | RHBDL2 | RPSA | GPR87 |
| FHL3 | MXRA8 | IPCEF1 | ERP29 | ZG16 | TRIB3 |
| STK16 | AURKA | FAM220A | SIK3 | NOTCH2NL | FNTA |
| SH3BP5 | LRRC42 | TUBA1A | TAF6L | HUS1 | RPL6 |
| MAPK14 | SRSF8 | MOG | BID | AMHR2 | PSMB5 |
| PABPN1 | RAB27A | CUEDC2 | CDK5 | VRK3 | NDUFV1 |
| SLC25A51 | TOMM6 | QPCTL | EGFR | CCL14 | GPR162 |
| PUDP | CMPK1 | TNFRSF6B | TAS2R45 | TAS2R8 | AUP1 |
| NKIRAS2 | PRKAG3 | GDF11 | C12orf76 | CSAG3 | TMEM116 |
| RAPGEF5 | MAPK8 | TTC12 | COX8C | RPL4 | IFNA6 |
| APOF | SLC39A9 | CDK9 | CFAP20 | ATP12A | MZT2B |
| NR2C2AP | GRK3 | SNX31 | TSGA10IP | CHAC2 | ERBIN |
| CDK16 | AKT2 | FRYL | GFOD1 | MGAT2 | OXER1 |
| PRRT1 | GPSM1 | TADA2A | PIM3 | CREB3L1 | AKAP7 |
| PIGH | NO_MATCH_209 | ARL6IP5 | TMEM231 | MIR7-3HG | HSDL1 |
| CTRB1 | RPF1 | SNAP47 | UBE2S | SOCS3 | CCDC172 |
| ORMDL2 | URB2 | POGLUT1 | C9orf40 | POLR2C | ZFAND2A |
| EGFR | HESX1 | HSD17B11 | P2RY4 | PIP5KL1 | MLIP |
| NUP62CL | KRTAP10-5 | RNF148 | P2RX6 | HNF4G | EPDR1 |
| CYSTM1 | CPO | CDK6 | PAFAH1B1 | C1orf35 | C8G |
| RELL1 | METTL21A | KIRREL2 | NOTCH2 | MMGT1 | BAGE |
| TMC8 | XLOC_I2_013503 | PYM1 | ADAM2 | LINC01366 | MYO1D |
| GCFC2 | UBQLN4 | BCAS4 | HK2 | LDLR | ANGPT2 |
| CTRC | CLK3 | NAB2 | DNAJB5 | VIM | NO_MATCH_218 |
| WDR5 | SLC17A2 | DYNLL2 | GPR148 | ARMCX6 | SEMA7A |
| SLC35C2 | RPS6KB2 | RPS19BP1 | OR5AP2 | RPS6KB2 | TMEM254 |
| HOXC11 | COPS7A | NO_MATCH_12 | STK3 | CPA6 | PRR4 |
| WFDC6 | NEK2 | HCRTR2 | SRD5A1 | OXGR1 | PPM1B |
| GCA | C1orf226 | INSL4 | SCAPER | HTR2A | UBE2D4 |
| C11orf74 | GNG2 | AGBL3 | TXNL4B | SUSD6 | POMK |
| APOC4 | Mchr1 | RNF111 | CHAC2 | KAT7 | AURKB |
| FBXO46 | GSTM3 | KRTAP10-9 | ATP6V1C1 | SULT1A1 | ALKBH4 |
| FGA | HSD3B1 | CDK2AP2 | CYP27B1 | CSF1R | PIP4K2B |
| GABRB2 | MAPT | TMEM71 | COMMD10 | PAQR5 | ZNF417 |
| RBMY1J | MAGEC3 | SPEG | ZG16 | PSG3 | RBSN |
| XLOC_I2_011027 | HIST1H2AK | ANKRD50 | MARCO | NDUFA13 | NO_MATCH_233 |
| SUMO2 | TNIP3 | HIBCH | C16orf52 | SLAMF1 | EMC2 |
| ST14 | EGFR | TEX19 | FAM216B | CPNE2 | G0S2 |
| ECHS1 | CFP | CCNDBP1 | TCL1B | PDCD1LG2 | TCEAL9 |
| TUBB2B | PCMT1 | PLEKHF2 | SGMS2 | MTX1 | OR1OS1 |
| ZNF24 | IDI2 | CD99L2 | MPP6 | SSUH2 | ACTC1 |
| PTGDR2 | MACROD1 | AMIGO1 | DYM | STK40 | SLC9A2 |
| PARVA | XLOC_003385 | LIME1 | C22orf29 | ZNF449 | WLS |
| PQLC2 | LINC01600 | RBX1 | SRP9 | PYHIN1 | NCSTN |
| CDR1 | ST6GAL1 | HLA-A | PYROXD1 | NO_MATCH_10 | SMTNL2 |
| SH3YL1 | COL9A3 | ABHD18 | NNMT | NO_MATCH_65 | CAV1 |
| PDLIM4 | FCRLA | WNT1 | IZUMO1 | GREM2 | IGSF6 |
| CDK5 | TSPAN12 | C21orf2 | NO_MATCH_253 | AVPR1A | STK32C |
| CLK3 | XLOC_I2_008285 | AK3 | NUP62 | PLEKHG7 | AEBP2 |
| PREB | TMEM231 | ARL10 | EIF5 | CNPY2 | MRGPRD |
| USP6 | NPAS1 | TNFRSF14 | HSD17B3 | LHFPL1 | OR5P2 |
| SVOPL | PEPD | BEND6 | ACKR1 | SAA2 | MRPS22 |
| LRRC8D | C15orf32 | PHLDB1 | FAM204A | CLK1 | CFAP47 |
| CNPPD1 | FAM127B | PTPRR | CD207 | LINC00305 | TGM5 |
| TSPAN32 | CGNL1 | GPR171 | PIKFYVE | FYCO1 | IFIT2 |
| CREB1 | ZC3H8 | USP32 | DDI2 | C5orf46 | FSD2 |
| LTB4R | HGF | Pin4 | H2AFY2 | TDGF1 | SLC25A51 |
| ZFP28 | C1orf112 | PIEZO1 | ITGB3BP | SSTR5 | SYPL2 |
| DUS2 | DDX55 | PREPL | PUS7 | PDILT | NUSAP1 |
| MKNK2 | RAB11FIP2 | TRAP1 | C6orf203 | SCAMP4 | HGF |
| TCF4 | PTP4A1 | MAD2L1 | BEX2 | NO_MATCH_137 | MYF6 |
| FAM167A | ACER1 | ACTG1 | STRADB | PLB1 | TMED10 |
| NO_MATCH_130 | RPUSD3 | LURAP1 | NXNL1 | UBE2G1 | TERF1 |
| OVGP1 | IL1A | TRIML1 | NECTIN4 | UCHL5 | TMEM129 |
| CER1 | ETV4 | RABL3 | GRIN2A | HMGCS2 | SLC25A35 |
| MRPL37 | ZDHHC7 | FAM46C | GTF2A1 | MIPEP | ATP6V1D |
| TMOD2 | CSNK1G1 | SRSF11 | RPS6 | RABEPK | BCAS1 |
| STK40 | NUDT10 | LILRB4 | ACSS3 | NAT8B | HCLS1 |
| LRRIQ3 | FAM69A | MMD2 | PRPS1 | JAK2 | CELF3 |
| RPS27L | LZIC | TAS2R42 | RELT | LGALS8 | SUCLG1 |
| RAB42 | IMPACT | RPL23 | ENPP7 | DUSP18 | FAM49B |
| TGFB3 | BCHE | PIP5K1A | ARMT1 | FOXN3-AS2 | ST8SIA1 |
| ANKRD36BP1 | TCEAL3 | BDKRB1 | HEY1 | B9D1 | RIMS2 |
| KCMF1 | C19orf43 | DCAKD | GPR20 | ATP6V0C | ID2 |
| ISCU | TMEM155 | TBATA | MFSD1 | CEBPG | TOLLIP |
| LFNG | CSNK1D | AUP1 | PHOSPHO1 | RAE1 | TMEM234 |
| NIFK | GPRC5D | ZFAND5 | ERP27 | DOC2A | GOLGA8EP |
| SNRNP40 | IL21R | TMEM25 | TEC | NME1 | OLR1 |
| MYOF | FBXW12 | HHATL | SMCP | TGFBRAP1 | C18orf21 |
| Zyg11b | NO_MATCH_29 | ARL6 | PARP3 | RFK | SF3B4 |
| PLPP1 | CXADR | NO_MATCH_57 | FTH1 | ZDHHC9 | BCL2L15 |
| SKAP1 | ATG9B | XLOC_013207 | BCKDK | TMEM72 | GRK6 |
| NACA2 | PROSER2 | CKB | LINC00486 | RPL24 | PSMD3 |
| GPATCH11 | SLC2A1 | AK1 | PCSK9 | DDX18 | LEXM |
| EPHA4 | APBA3 | PRSS50 | DRD5 | ZCCHC7 | FAM19A3 |
| NOSIP | PLEKHJ1 | Cdk11b | VPREB3 | OR52B2 | HOXB1 |
| CMA1 | SCG2 | ERMAP | ADCK2 | NO_MATCH_85 | TTI2 |
| POLR3D | FSHR | MMP12 | SERPINB5 | TMED4 | CCDC66 |
| M1AP | ZNF526 | KIAA0753 | BBS9 | NO_MATCH_60 | C17orf62 |
| P2RX4 | LOC100289561 | ANKRD34B | MAGED4B | ACTR3B | OR2S2 |
| TPBG | RWDD3 | C11orf71 | UQCRC2 | TRIM42 | NR0B2 |
| IGLL1 | MRPL17 | PRR14 | HIST1H3E | PROCA1 | PDGFRA |
| LANCL3 | TXNDC12 | CST8 | CTDNEP1 | GAL3ST3 | PRPS1 |
| SUV39H2 | NIPSNAP3B | TNFRSF11B | CTBP2 | GDNF | MRPL1 |
| STK40 | BOD1L1 | CDH5 | LYNX1 | ARCN1 | PSME2 |
| TAS2R38 | MAPK3 | FASTK | PTGER4 | Grid1 | PCBP3 |
| RPL21 | IQCH | NUAK2 | GNB1 | MTRF1 | TOPBP1 |
| RCAN2 | RASGEF1B | RPS6KC1 | SMARCD3 | SGK2 | TMEM62 |
| TMEM255B | MROH8 | GPR143 | POLR2E | HCAR2 | MAPK10 |
| RET | ZBTB44 | SLC19A2 | GJB5 | MDFI | PTBP2 |
| HLA-A | MRAS | C11orf45 | PRKAB2 | SEMA4D | SETBP1 |
| PDGFB | CCDC9 | AKAP13 | UBE2DNL | 42984 | PDXK |
| GPR35 | ADIRF | ACOT4 | RPS27 | CAMK4 | MGC16025 |
| KLRG2 | MAP2K2 | OR10G8 | STRADB | FUT1 | DUSP12 |
| KLK5 | MAPT | AIMP1 | FSTL4 | TMEM30A | AGBL2 |
| UTP15 | CLEC4C | HIST2H2AA3 | VTCN1 | COX16 | CST6 |
| RIC3 | SPEM1 | SIX1 | RPS28 | ST3GAL1 | PLA2G7 |
| RELL2 | SERTM1 | RAG2 | SNAPC3 | MNS1 | DNPH1 |
| CCDC74A | NOG | CA11 | IL12RB1 | AIMP2 | CLN8 |
| NAT1 | TOMM40L | HIST2H2BF | SPDYE11 | TNFRSF9 | RPLP0 |
| SPPL2B | MAP2K2 | LONRF1 | TMPRSS12 | METTL2A | BCL2L14 |
| C16orf74 | YY1AP1 | CHCHD2 | TMEM164 | MPP6 | ATMIN |
| RGP1 | DNAJC30 | C15orf40 | RUSC1-AS1 | CHST5 | PBRM1 |
| Mbd5 | EXO5 | RORC | HIST1H4H | FAM64A | ARL11 |
| STX19 | XLOC_I2_006862 | CDK10 | FGFBP2 | DEFB104A | NLGN3 |
| GIMAP6 | ZNF148 | PDXDC1 | KLHDC2 | SNAPC3 | ALDOA |
| CABP5 | DGKE | MMP13 | ZNF543 | YBEY | CHRNB2 |
| GDPD5 | TRMT61A | SEPHS1 | BRE | TMCO2 | DCDC2B |
| OGT | GMCL1 | SAT2 | TSSK4 | CALM2 | UBXN10 |
| ELOVL7 | CLEC4A | BORCS8 | LGALS3 | CELA2A | ALKBH2 |
| DEXI | FGR | SLC22A2 | NPIPA1 | RNF10 | GTPBP3 |
| FLT4 | TWISTNB | LHX8 | PPARG | ZXDC | RNASE13 |
| VASH2 | NO_MATCH_166 | CDK20 | GRM2 | POLR3K | OR4D1 |
| QRFP | CSNK2A2 | FBP1 | MRPS12 | EIF2S1 | LMBR1L |
| DCTN3 | FURIN | C7orf43 | NO_MATCH_183 | SRP68 | LOC440700 |
| SLC10A1 | AGTR1 | SLC22A7 | SIGMAR1 | P2RY13 | SLC41A3 |
| NCALD | PCP2 | PRSS2 | SLCO4A1 | PANK1 | RAD54B |
| CELF4 | DPP10 | CFLAR | FUBP3 | CISD1 | GALR1 |
| TIMM8A | PIGK | CFAP45 | MSLN | BRI3 | CHCHD10 |
| ASCC2 | PDK4 | C1orf131 | C1QTNF3 | CEP135 | P2RY12 |
| SMIM2 | UPP1 | GPRC5D | SIGLECL1 | CLHC1 | OCIAD2 |
| SGTB | PRG2 | C9orf139 | MUC1 | FAM122C | ACOT1 |
| AK4 | ZNF581 | METTL9 | LNX1 | TDRD10 | CCP110 |
| ZNF80 | PI4K2B | KLHL15 | HSD11B1 | PLA2G3 | PPAN |
| CENPO | NAA60 | CD300E | RTN4R | GTF2IRD2B | BTBD1 |
| HNRNPDL | IGLL5 | MAP2K2 | TMEM123 | C22orf31 | PRKCH |
| AKR1B10 | KDF1 | TMEM35B | DGUOK | SAE1 | SAMD11 |
| ZSCAN2 | NO_MATCH_46 | RPL13A | SLC9B1 | ATF2 | FOXRED2 |
| RASSF2 | HIST1H2AM | BMPR2 | ASB6 | LY6K | HIST1H3F |
| HSD17B10 | E2F6 | PDK1 | FAM110D | HLA-DMA | NAP1L5 |
| PTGES3 | B4GALT3 | XLOC_004271 | PTGES | SPTSSA | TPM1 |
| GFAP | CNRIP1 | RAD51 | MBLAC2 | C11orf54 | KCTD1 |
| MAZ | UBL4B | CYSLTR2 | KIAA0141 | PTK6 | MVD |
| ARAF | ZFHX3 | SUDS3 | ZHX1-C8orf76 | ZNF644 | PPIA |
| EIF4A1 | NO_MATCH_201 | NO_MATCH_87 | PHKG2 | TMEM189 | RRP7A |
| PDE1A | VEPH1 | TMEM106B | AIRE | MRPS6 | LOC641367 |
| RHOF | ROPN1 | THRA | CLDND2 | SLC17A4 | Cpsf3l |
| P2RY13 | RGCC | SERPINB5 | MFN1 | LOC105372824 | OR2T33 |
| GALNT9 | PCDHB4 | CCL2 | PRKCI | HIST1H4C | RPS17 |
| DMRTB1 | C11orf70 | SPACA3 | LGALS3BP | NSMCE4A | HECTD3 |
| PARP11 | TP53BP1 | BLK | MLC1 | CAMK2A | LGALS7B |
| CNTF | CCDC84 | KLK15 | SUPT3H | ANGPTL3 | TMEM82 |
| MOB3C | RNF183 | KRIT1 | TCF23 | SCTR | KRTAP19-1 |
| IKZF2 | CELA2A | HMG20B | GRIK2 | SCYL1 | PSG2 |
| PRR3 | STK19 | HLA-DRB1 | PPP2R5C | CLEC12A | GLRX3 |
| RNH1 | CXCR3 | SNAP29 | DGAT2 | MZB1 | LGI2 |
| CD274 | DOK1 | NPEPPS | ZMAT3 | ZKSCAN4 | CLEC11A |
| UBE2T | TUBA3D | HAGH | GPSM3 | NECAB1 | KRT81 |
| PEX2 | PARPBP | LINC01588 | KMT5A | NAXE | TESK2 |
| LIN52 | MTHFS | HYOU1 | SKA3 | C5orf38 | WNK1 |
| RPL14 | ITGA6 | CATIP | NOXRED1 | GABRG2 | PRR11 |
| IL36B | GJB3 | MGLL | OR2A12 | NO_MATCH_190 | CERS3 |
| ZIM3 | CCNJ | MAP4K4 | EXOSC7 | SYS1 | LSM6 |
| OR7A5 | PTH1R | FAM58A | TAS2R19 | SCEL | CXCR1 |
| NPL | MOS | BMF | SRP14 | SREK1IP1 | CRISP3 |
| TMX1 | CAMKK1 | GSTA4 | RDX | RPE | DKK4 |
| EIF3I | TM2D3 | NOVA1 | OR10A2 | KCNJ11 | MC5R |
| CCKAR | B3GAT3 | DACH2 | FRMD3 | DRD5 | BSCL2 |
| TUBGCP3 | TMEM88 | RIPK2 | GDPD2 | SSBP4 | XLOC_001866 |
| KRT15 | ACSF3 | IFNA17 | LINC01260 | PACSIN3 | MCM7 |
| XLOC_I2_005978 | HIST1H1E | FGGY | MOB1B | LRRC15 | ADGRG1 |
| METTL15 | FXN | GPR18 | CREB3 | KRT71 | APLN |
| NO_MATCH_187 | GPATCH4 | SEC63 | DFNA5 | LDLRAD1 | PCDHB10 |
| TMED5 | KIR2DS4 | ADIPOR1 | IQUB | C1orf54 | CDK6 |
| GCM2 | LGALS4 | LOC442132 | GGTLC2 | LIG3 | RPL9 |
| ANAPC5 | MAPK14 | CCR7 | FUT9 | FSBP | EXOC8 |
| NO_MATCH_48 | TLDC2 | USP18 | NSUN5 | EPN1 | ANKRD13D |
| OR14I1 | TADA2A | C3orf14 | PRAMEF5 | TAAR9 | MCTS1 |
| CST8 | COX14 | ADGRE2 | ETV5 | DDR1 | STARD7 |
| AP1S3 | TBK1 | TAC3 | MTHFD2L | MAGT1 | GGA2 |
| MYCT1 | EXOC5 | ZFYVE27 | PSMD14 | RPS16 | GPR52 |
| MAPK14 | VPS52 | CST2 | TPM3 | AGA | F2RL2 |
| FKBP14 | RADIL | P3H4 | EIF1AY | PLIN2 | RGR |
| ANAPC11 | EAPP | ECI2 | THAP6 | EEF1D | EGFR |
| RAB17 | RXFP2 | SNN | LGI3 | G3BP1 | TREML4 |
| PDIK1L | HEYL | UAP1 | ILK | SYCN | FHL1 |
| C17orf97 | TMEM67 | ACSBG1 | SLCO4A1-AS1 | ARL17A | PUSL1 |
| CRHR2 | IGF2 | LIPC | MUC1 | USP32 | MTHFD1L |
| BBS5 | ADGRF1 | LOC440570 | EVA1B | DDX39A | FAM214B |
| RGS22 | CYP4F2 | CST4 | CRYZL1 | AGBL4 | STMN4 |
| C2orf68 | MT1E | DDX28 | GLT1D1 | PCED1B | IL10RA |
| DBNDD1 | CD80 | ATP1A3 | MAN1B1 | TBC1D3J | ZDHHC2 |
| DDX50 | TRIM29 | ZMYND19 | ILK | SALL2 | CYP2C9 |
| HARS2 | MAGEB2 | FOS | TMSB4XP8 | ZNF621 | PSMB5 |
| DRG1 | MET | FGFRL1 | TMEM70 | FCGR3A | CPVL |
| PAQR8 | BEX5 | PF4 | MSRA | GPR20 | IPO5 |
| PRKACA | SERHL2 | KIAA2013 | PSMA6 | CHCHD7 | TFAP4 |
| MOB1A | NR1I3 | CALCB | JPH1 | LCP1 | UBE2R2 |
| TNFSF12 | MLKL | NMRK2 | EGFR | NO_MATCH_95 | MAGEA5 |
| PLLP | TWISTNB | ARHGAP5 | UGT2B4 | EIF2AK3 | OPALIN |
| CYSTM1 | ZNF384 | FGB | CSHL1 | DXO | HP1BP3 |
| RUNDC3B | ULK3 | MUC20 | PADI3 | SLC6A3 | ALOX15 |
| TDRD3 | MET | CHCHD6 | INTU | SNAI3 | ALKBH8 |
| OR3A1 | SLC2A3 | RIC3 | KHDRBS2 | SCG3 | LZTS2 |
| GPR34 | SLCO1B1 | Rictor | DUSP1 | DGUOK | C7orf49 |
| FBXO48 | KRTAP19-7 | SIAE | MRGPRG | PGR | AKR1C1 |
| FBXL21 | SLC25A36 | UBE2Q1 | TESK2 | HTR5A | ZKSCAN3 |
| VAMP4 | GNAL | ZFYVE9 | MAPT | IL23R | MTPAP |
| RNF32 | IKBIP | P2RY11 | SAMD10 | HACD2 | LY6G6D |
| SH2B3 | CACNG3 | SIRT6 | SUCNR1 | HPRT1 | SERPINA3 |
| HDAC11 | ADAM30 | EMSY | CPSF4 | CARD11 | FLT1 |
| CHD9 | LRRC40 | KCNJ10 | GABBR1 | FAM168A | PHYHD1 |
| MRGPRX4 | PCGF6 | PCNX4 | ZNRF1 | ACKR4 | TNNT3 |
| SARNP | RPL17 | ADGRL1 | DNASE1L1 | LRRC20 | ACBD6 |
| CLDND1 | PPP2R3C | DLEU7 | DTD1 | ARAF | LYZL1 |
| TK1 | RNF170 | CRB3 | RAB33A | OR2T10 | LY86 |
| COG6 | SRR | LYPD2 | OR2T27 | RPS18 | MTMR7 |
| DENND2C | HIPK1 | FBXO22 | PARK2 | NEUROG1 | CD72 |
| U2AF1 | A1CF | BET1 | NEK2 | SREK1 | MBTPS1 |
| TBCA | GRK3 | FAM57A | PPP4R3A | TTC6 | HRH4 |
| OGN | TUBAL3 | GLTP | TMC5 | USB1 | DVL3 |
| SMU1 | SLC23A1 | RPSA | STK32C | SULF2 | NDUFB6 |
| FGFR1 | MTIF3 | LCN1 | TBC1D9 | FCGR2A | ZG16B |
| LIPH | CHRM3 | FBXL8 | THEG | NO_MATCH_272 | UBE2Z |
| TRDMT1 | RGS20 | DYNLL1 | SEMA4F | GSG1 | ERCC6L2 |
| SEMA6C | COPRS | DEFB128 | CCNY | CAMKV | TTC5 |
| LMO2 | OR2B2 | HBZ | VWA3B | LRRC37A5P | RNF19A |
| FAM153A | PRKAB2 | NRBP1 | C5orf60 | VPS37B | DPYSL2 |
| NSUN5 | LCK | GLB1L2 | XLOC_011297 | CD70 | PDIK1L |
| ALDOC | RACGAP1 | SERINC2 | ZNF287 | ZNF19 | CLDN20 |
| ANAPC16 | ISCA2 | ABHD14A | COL8A2 | ABCB5 | SCAP |
| FAM92B | KRT222 | MFSD10 | EDNRB | TCP11L1 | RPL13AP17 |
| GPR151 | ISG20 | RSL1D1 | FAM222B | NIN | RPGRIP1 |
| SHARPIN | RUNDC1 | CFAP161 | WDR60 | ABHD14B | NO_MATCH_58 |
| MID1IP1 | CCNH | PPIA | RRP8 | NO_MATCH_213 | NDUFB3 |
| PRKACA | KCNC1 | ICE2 | SMPD1 | TNIK | UCP1 |
| LINC01547 | BTBD6 | FGFR3 | RHOT2 | CALM3 | GPR87 |
| EGFR | KRTAP10-11 | DIO1 | PLA2G2A | LOXL2 | OXTR |
| ADRM1 | MRPL45 | MEAF6 | ZBTB49 | FMC1 | XLOC_I2_009136 |
| GPR52 | RNF114 | STK17B | RXRG | HEATR9 | NUMA1 |
| CHRM5 | STK16 | CDKN2D | CUTA | GNG3 | C1QL4 |
| TMEM169 | PON1 | NR4A1 | GRM8 | CHD1 | RIOK1 |
| PRKAA1 | HBG1 | RNASEH2B | MVB12A | SLC25A27 | ERBB3 |
| COPZ1 | FGGY | TBC1D26 | FOXP3 | HPX | MAPK8IP3 |
| WDFY1 | KPNA2 | NAT14 | KLHDC1 | PTPN9 | LACRT |
| PDRG1 | KRT8 | PRICKLE4 | PBK | CSRP3 | NYX |
| SLC25A10 | COMT | ZFYVE19 | TNFSF13 | ASPM | ADCK2 |
| WASL | TMEM39B | EGFR | KHNYN | NO_MATCH_141 | PLA2G10 |
| UCHL1 | DEFA4 | CCL7 | Kdr | C10orf71 | BMX |
| MCHR2 | SPATA8 | ABCC4 | CT45A10 | KLK3 | NO_MATCH_26 |
| SMNDC1 | SUPT5H | TAAR2 | F2RL1 | APOBEC3G | TH |
| ANXA2 | KLK7 | CDC42SE2 | SLC2A5 | POLR2K | LIPE |
| BOC | C18orf25 | NMUR2 | KRTAP26-1 | AP4E1 | AHDC1 |
| CIB4 | TBC1D28 | TLDC1 | MGST3 | CCDC57 | RAB39A |
| NPR2 | TAF8 | GUK1 | IFT80 | UCK2 | ATP6V0C |
| KCTD17 | CIPC | PI3 | EREG | CNR1 | ABHD4 |
| PARP6 | ST3GAL2 | PRPS2 | HSPBAP1 | TMEM86A | METTL13 |
| NO_MATCH_161 | SNX3 | FXN | SNRPF | CAMKK2 | ZNF223 |
| OR1D4 | CPXCR1 | MGST1 | SAMD7 | SLC2A6 | CAPNS2 |
| STAG3L2 | RPL26L1 | ZIC3 | AKIP1 | MGC34796 | TCEAL4 |
| FZD4 | LRRC10 | SGK494 | AMY2B | CRYBB1 | IDO2 |
| PROK2 | C22orf46 | KCNH1 | CCDC83 | ATP1A4 | ADGRD2 |
| CC2D1B | SPATS2 | CCR7 | FLJ13224 | UQCRFS1 | GSTP1 |
| GLUD1 | RLN2 | BRK1 | CHIC1 | AADAT | PRSS2 |
| ALG6 | DDAH1 | GNB3 | USP49 | GRINA | KIF14 |
| ASL | DZANK1 | PSD4 | INPP4B | TRIB1 | ADAMTSL1 |
| SLC45A2 | SRD5A3 | RPS4X | ZNF550 | Olfr981 | CLRN3 |
| Dolpp1 | XLOC_007690 | RITA1 | ADORA2A | SYTL2 | ACVRL1 |
| NPM2 | ILK | PCDHB15 | CXorf57 | SLC6A1 | NO_MATCH_5 |
| OLFML3 | CCL15 | SLC13A5 | FAM175B | CDCA4 | B4GALT7 |
| FAM96A | TTC39C | ELOA3B | DOLK | PGLYRP1 | TLX3 |
| JAML | TTC39B | MS4A1 | FAM163A | NOP16 | AVPI1 |
| NUDT2 | ALS2CR11 | CT55 | LYSMD2 | NT5C | EGFR |
| MRPL10 | RSPO1 | ZC3H3 | MICU2 | CCNG1 | NO_MATCH_63 |
| LEMD1 | NT5DC2 | OR13C5 | CFAP77 | NO_MATCH_281 | OR52N4 |
| MRPL10 | NO_MATCH_160 | ZNF474 | PRELID1 | CLDN9 | C22orf23 |
| RPL26 | TMSB4X | HES6 | LRRC55 | RRM1 | RASSF4 |
| ERCC2 | RGS2 | TMEM230 | MRGPRX2 | STK17B | MFAP3L |
| GDAP2 | SF3B3 | PSMC1 | NDUFB6 | IFNA7 | KIAA0232 |
| LSM4 | SLCO1B1 | ZNF346 | THEM4 | IGFBP6 | C14orf105 |
| HRH4 | HSD3B2 | MOCS3 | MSMO1 | TXNRD2 | SLC5A9 |
| LBX2-AS1 | FXYD3 | NAGPA | FKBPL | IFI30 | MYL12A |
| LOC105371303 | SMIM5 | RSPH14 | NO_MATCH_82 | PAPD7 | TAS2R20 |
| NSDHL | LSM14B | MRPL16 | ISG20 | ACVR1B | MILR1 |
| COL9A1 | MPPE1 | BRIP1 | BRICD5 | SYNE4 | PRKAB1 |
| TAS2R60 | DDX21 | MAF1 | HIST1H4G | EEF2K | CHCHD3 |
| SLC5A2 | DTL | RAB28 | TTC9B | TP53RK | NO_MATCH_140 |
| APBB3 | ADGRF1 | DPM3 | UQCRH | QTRT1 | MRGPRE |
| CNPY1 | TCF19 | TRMT11 | NSUN5 | CSNK2A2 | SGCZ |
| PRKCI | CDC42EP4 | LHFPL3 | PRAC1 | HYAL3 | CFAP46 |
| PGR | NT5C | SULT1C4 | OSTN | ATP5F1 | SH2B1 |
| LOC107984064 | XLOC_I2_000915 | SLC9A8 | ZNF519 | SLC41A3 | PON2 |
| TMEM81 | C9orf116 | CXCL14 | PKLR | FAM71E1 | RHAG |
| XLOC_012148 | PSMA5 | EFCAB3 | SPTLC2 | EBLN2 | CER1 |
| PDZD9 | GLT8D1 | TMEM216 | GRM5 | MLH3 | SHISA5 |
| FCRL2 | XLOC_010007 | USP29 | GCC2 | GOLGA7 | HAX1 |
| WBP1 | PEX11A | NO_MATCH_219 | LIMS1 | CETN3 | TSSK1B |
| ARHGAP45 | PRMT2 | ACTA2 | C17orf49 | KIT | DRD3 |
| LXN | RMND5A | NDEL1 | OTOR | SNRPD1 | TSPAN10 |
| CWC15 | MYZAP | SLC25A48 | FERMT2 | F11R | XLOC_I2_001972 |
| LOC107984344 | TMEM220 | PEX7 | CDKL3 | CASC4 | RBFOX2 |
| SPPL2B | PIK3CA | DPPA2 | SORCS1 | ZCCHC11 | DSCR8 |
| CCR1 | BCAR3 | SWT1 | RBL1 | 42796 | ISYNA1 |
| ZNF26 | NO_MATCH_241 | CXorf57 | PPP2R5D | VCP | LOC107984065 |
| ST8SIA2 | TUBA3FP | SIGLEC5 | KDR | SLC4A2 | LAIR1 |
| HIST1H3G | ACBD7 | PF4V1 | ITM2A | GCM1 | DNAJC14 |
| NCLN | NEU1 | ADI1 | ECEL1P2 | HIST1H2AC | ACP5 |
| UBE2I | MSRB2 | MAGEC2 | CATSPERB | RBM25 | RPL18 |
| BMT2 | DGKE | B2M | Eri3 | HNRNPF | SPATA19 |
| ADAT3 | VPS26A | PRKRA | DTD1 | CCDC110 | GPR155 |
| PAPOLB | RNLS | DICER1 | DCUN1D1 | TMX2 | CCL17 |
| PLXNA2 | GPR174 | SEC16B | CD164L2 | NMNAT2 | LCN15 |
| NDUFAF8 | KANK1 | LIPA | RBFOX1 | GRM7 | C14orf177 |
| NSL1 | HTR3D | CYP19A1 | IL31 | VPS41 | GPR52 |
| C11orf65 | EDNRA | PHACTR1 | STK19 | GPR27 | HAT1 |
| MOS | PTPRO | AXDND1 | MPST | ZNF385C | LRPPRC |
| EIF2AK4 | TNKS2 | LOC105369201 | PRKCH | ERBB3 | SLC2A4 |
| TEAD4 | DYRK2 | MYO1F | ALDH1A2 | ZNF462 | CX3CR1 |
| TMEM170A | GAL3ST1 | LCN8 | NTRK1 | SIRT6 | PNKD |
| CSNK1G2 | CCDC158 | GALNT1 | C11orf53 | ANKS1B | RNF185 |
| NO_MATCH_227 | SLC8B1 | RPS4X | AGTRAP | UPP2 | ADGRF3 |
| COX7A2 | AURKC | FIGNL1 | IL21R | CLEC2D | TESK2 |
| HLA-DOA | ZDHHC11 | CST9 | TECR | LPGAT1 | SHISA2 |
| TTC33 | DNAJC4 | NR1I3 | TAS2R41 | RAD54L2 | Akt1s1 |
| OLR1 | ADGRG7 | JUNB | XLOC_003758 | CDC42EP3 | CHAD |
| PFN2 | CPSF4L | ADRA2C | TMEM40 | NEK5 | FCGR2B |
| SGK494 | ANKAR | KIF26A | CFHR4 | CBY1 | MAPT |
| TSC22D4 | VCAN | JAG1 | DLX1 | TMEM55A | TRIM27 |
| INSRR | ZNF207 | NADK2 | AIMP2 | RPS6 | ACVRL1 |
| GPR63 | FASTKD3 | CUL3 | GPR68 | PRKAB1 | NPY6R |
| GPR173 | BCAP29 | PRSS38 | MKNK2 | NANS | NDST3 |
| CLCN2 | PRPF4B | CDC42EP4 | OTOA | AGRN | CDH26 |
| RAB39A | EFCC1 | CKAP2 | RAB20 | TMEM267 | CKS1B |
| FAM71F1 | TNNI2 | TMEM126B | PAPD7 | GRM1 | PRPF4B |
| IPPK | SWSAP1 | LNX2 | DHX8 | CD69 | NDUFV3 |
| ELOVL7 | PPY | STRN | RAB9A | DLD | BSPRY |
| UEVLD | CYTH4 | ZDHHC1 | NO_MATCH_7 | PI4K2B | TRIM39 |
| ALG8 | RBM15B | KIT | GRIN2A | CALM3 | TBXAS1 |
| RBP3 | HOXC8 | NGEF | SLFN5 | DAP | LUZP2 |
| MAPK12 | CLSPN | GPR155 | ARHGAP12 | CER1 | FAM71A |
| S100A8 | KIAA1143 | IL10RB | RPL13 | CYP3A4 | CENPS |
| GMFG | ECE2 | NO_MATCH_23 | FASTK | CTNNBIP1 | SDR9C7 |
| AFF2 | PIGN | ATAD3C | CYP1B1 | SAA1 | DTWD1 |
| TSSC1 | FAM104B | HIST1H1T | CDK9 | PHTF2 | SERPING1 |
| YPEL3 | NO_MATCH_198 | Ccdc28a | NMT1 | PCYOX1L | MOXD1 |
| RETNLB | ART4 | PNO1 | LIMK1 | RHO | AURKAIP1 |
| MRPS27 | IL1RAP | ZNF431 | Bloc1s1 | FLJ44635 | NOTCH1 |
| AMMECR1L | TMEM185A | TBK1 | FARP2 | MRGPRF | ARMC12 |
| TLCD1 | TMCO3 | RGMA | PLAC1 | FBXO6 | DSN1 |
| CLEC4M | ALS2CR12 | HMG20B | NPR1 | RFESD | EPHA2 |
| CDK1 | INPP1 | ANAPC5 | F2R | ZNF616 | CFAP47 |
| MEPCE | FGF12 | PIK3R4 | DIS3 | PARS2 | NO_MATCH_274 |
| OIT3 | MRPL11 | GAD1 | ADGRE1 | STK24 | ECSCR |
| CACNA2D4 | GNLY | TIMM21 | SDHB | HSPA14 | GAR1 |
| ADGRB1 | ZNF615 | CYP27A1 | SPANXC | FAAP20 | DAPK1 |
| MERTK | BTRC | TRUB2 | DDI1 | CD200 | FABP9 |
| CNDP2 | AK1 | LCOR | P2RX1 | GRM1 | NXPH1 |
| CLCC1 | CCDC110 | NOTCH3 | SLC38A4 | KLHDC9 | VSNL1 |
| Ttyh1 | POGZ | COQ8A | EPHX3 | GPR21 | UBAP2L |
| PSG11 | STXBP6 | ASS1 | SSR4P1 | SCAF8 | CAMK2A |
| PNLIP | PAQR8 | ERBB4 | PTK2 | MS4A2 | EXD3 |
| OR5F1 | HHEX | EIF4H | TM7SF3 | MATK | USP15 |
| PCP4L1 | SYT16 | SERPINA1 | BCKDK | CACNA2D3 | SLC35E3 |
| SLC22A9 | CASP3 | COA7 | EMIDI | PRSS22 | GLYATL1 |
| ARHGEF2 | AHNAK2 | MEA1 | TTBK2 | MESDC1 | DZIP3 |
| NME3 | OPRM1 | GCHFR | MPZL2 | POLR3GL | KCNA1 |
| OR8B4 | PCNA | LIN7C | NHLRC2 | PSMB3 | LHPP |
| NCS1 | CNTNAP3 | ZNF575 | NXF3 | GNB5 | STK26 |
| XLOC_I2_005718 | RUVBL2 | C16orf70 | MOGAT2 | ALKBH7 | COX6A2 |
| POU2F2 | CLCN6 | KLK12 | DNTTIP2 | COX18 | TALDO1 |
| SNX20 | HSPBP1 | PRPF4B | SLX4 | CCL24 | REPIN1 |
| ANTXR1 | RANGRF | EXOSC4 | BBS1 | CHMP3 | CD28 |
| CAMKMT | SPTLC1 | CHRNA1 | TXNDC15 | PLCB2 | OR13C3 |
| INTU | CYSLTR1 | LYPD2 | NPDC1 | CD3E | CA8 |
| IGFL3 | ACKR4 | GPRIN1 | RTP2 | CYP1A1 | PPFIA1 |
| ZNF595 | OR52B4 | ADGRA1 | INTS6 | HK1 | NO_MATCH_200 |
| TNFAIP8L3 | CDK10 | COA5 | FAM118A | RNF166 | DEFB106A |
| TLR10 | KRTAP23-1 | KIF6 | RPL23A | CDK5 | ASPHD2 |
| CYB5R1 | PHLDB1 | SEMA4G | TSPAN17 | CDK15 | OR2D3 |
| WDPCP | NAV1 | SPO11 | C9orf85 | FGFR2 | HERC4 |
| JAK2 | FGL2 | KIAA1161 | ZNF789 | NO_MATCH_226 | SLCO1C1 |
| PFKFB3 | ABL1 | ETFDH | SC5D | FUCA1 | TRMT10C |
| DUSP18 | ANXA11 | IRF3 | HACL1 | REG3G | KDELR1 |
| NIPAL3 | S1PR3 | ZNF30 | CLDN2 | NINL | CERS3 |
| DXO | OGFOD2 | BCL2L10 | OLA1 | PDCD1LG2 | NFATC2IP |
| ACVR2B | HINT2 | MCF2L | BANF2 | AREG | XLOC_I2_007111 |
| MEN1 | SLITRK3 | ACYP1 | PADI3 | DHX33 | C11orf16 |
| USPL1 | PIK3CA | TFR2 | TMED3 | DTYMK | TRPC7 |
| RAB40A | TPM4 | KIR3DL2 | CD160 | SENP5 | TRMT13 |
| TSPY1 | RPS6KC1 | CYYR1 | TSLP | COPS6 | SEC61A2 |
| DLG3 | IL17B | NO_MATCH_208 | ALPK2 | FZD6 | FAM189A2 |
| ZEB1 | CSAD | PLP1 | HMGB2 | ASAP3 | AMY2B |
| C17orf75 | ZBTB20 | SFXN2 | UPP1 | MFSD13A | IL33 |
| ERI2 | CSPP1 | USP42 | MRPL23 | HLA-DMB | UCN3 |
| RAD1 | CEP72 | BTBD16 | CCDC59 | RMI1 | PRR13 |
| 42988 | RTN4 | TRMT10B | XRRA1 | RRP1 | INPP5J |
| MRPL41 | TARSL2 | PTS | SLC39A2 | KCNV2 | NPC2 |
| FGL1 | CHRNA6 | VDR | CACNG7 | ZACN | NHP2 |
| NLRP10 | IPPK | NKPD1 | SCRN3 | WDR83OS | MYLK |
| MSH2 | XLOC_009142 | TMEM11 | PANK1 | TMEM222 | CSNK1A1L |
| TPP2 | NRF1 | HAUS8 | PRDX3 | TAS2R41 | NAPRT |
| PIH1D2 | PUS7L | MAF1 | VAPB | DDB1 | MEGF10 |
| NCOA3 | TSC22D4 | BPIFB2 | LYSMD4 | AFAP1 | ARMCX5 |
| ZC3H10 | ANO6 | FKBP9 | VAMP1 | ATG4D | MMP10 |
| EFNA4 | HTR3D | EIF2B2 | HIST1H2BM | TPPP2 | OR4N4 |
| UBE2Q2 | SPZ1 | ACTL6B | TAAR5 | COASY | REEP5 |
| FAM20A | NO_MATCH_11 | MAP2K2 | HIST1H2AA | HIST3H2A | PRB3 |
| DYRK4 | MAP3K5 | NPIPB9 | RARRES2 | HHEX | CCDC150 |
| AVPR1B | TSC2 | SMARCA5 | GSTT1 | ACVR1C | LOC102725035 |
| GPR137B | FBXO44 | NEK3 | UHMK1 | COL23A1 | NUMB |
| SPATA31E1 | LY9 | Fam76b | EGFR | C11orf65 | LRRC29 |
| SNRNP27 | CHMP6 | MACC1 | SV2B | RBMS3 | RXRB |
| CLIC5 | MIA2 | ATP23 | IQCH | LOC149950 | NRIP3 |
| BLCAP | HSP90AA1 | USP25 | HSP90B1 | SPAG11B | OSBPL8 |
| FGFR3 | LIMK2 | C2orf48 | NME1 | ANKRD26 | KDELC1 |
| DDIAS | CA14 | ERCC5 | STOML3 | TMEM165 | PIK3CA |
| FPGT | CCRL2 | NBPF9 | DCAF12L2 | FFAR4 | DTX2 |
| KCNJ10 | NICN1 | MRPS24 | ALPK1 | IGF1R | MCOLN3 |
| KIAA0922 | STAM | KLHL9 | TTLL4 | NTNG1 | ACSL3 |
| SLC48A1 | ZNF263 | KRTAP19-5 | MEIS2 | DUSP6 | TIMM10 |
| MPZL2 | KRTAP10-3 | YPEL4 | OPN5 | CD8A | TSPYL1 |
| CCNC | KDR | EPHA3 | CNBP | CCL4 | INO80E |
| HPS5 | ITLN1 | SLC25A39 | RTN4IP1 | TNFRSF9 | GPR15 |
| NOL6 | PRKAR2B | ZGRF1 | 42982 | LCOR | KRT26 |
| ANKRD27 | TSTD2 | ZNF260 | FAM19A4 | SSSCA1 | ZNF738 |
| KCNJ14 | CASP1 | GGT2 | TAS2R9 | ANXA2 | SLC35G5 |
| MAFB | LINC00895 | FAM208A | DNAI2 | SORCS3 | OR6T1 |
| FAM135B | ULK2 | NHLH2 | GPR173 | CCDC42 | HIST1H4A |
| ZNF610 | TOB2P1 | SBSPON | ADCY9 | ADGRA1 | IMPDH1 |
| NCOA3 | GNAZ | XPO6 | AHSA2 | MIR31HG | RPSA |
| LRRC10 | ZNF473 | PCDHB1 | PIH1D3 | LITAF | LINC01105 |
| YIPF4 | NRN1 | RPS6KB1 | LATS1 | PDZD4 | MAP4K2 |
| NRSN1 | PEX11G | XLOC_I2_004840 | CRYAB | ZNF574 | OR11H12 |
| SHMT2 | MPP5 | TPK1 | HRH3 | Fbrs | ANXA2R |
| MTHFD1L | ALPP | HOMER2 | KLF7 | STT3A | HRH2 |
| PNCK | ATF7IP2 | OR1S2 | PDE3B | KCTD21-AS1 | IFT80 |
| AQP7P5 | HSPH1 | RBM15 | MET | LIPC | Cdk13 |
| TMEM168 | KISS1R | PON1 | 42803 | LHFPL1 | TSPY26P |
| NUP155 | KCTD17 | PNMA5 | TMEM214 | UPP2 | TBC1D10B |
| GPR65 | WEE2-AS1 | HDAC10 | GYS1 | THY1 | ACLY |
| SPIN4 | MC3R | RBM10 | FBXO38 | C9orf24 | PCSK1 |
| CORO1B | LATS2 | B3GALT5-AS1 | CACNG1 | TM2D1 | ADGRB3 |
| TMC6 | ABCC3 | TRIM74 | C1QA | LIN37 | HNRNPU |
| LRR1 | DLGAP2 | DYNC1I2 | MCM2 | TEX11 | TBRG1 |
| PNMT | CCL13 | DAZAP1 | SLAMF1 | PHYHIPL | BAGE2 |
| UNC45A | MMRN1 | RXFP1 | IARS2 | TRAPPC8 | CUL4B |
| UPK1A | STARD5 | PITHD1 | ABHD12B | LRRK1 | SCMH1 |
| GSTCD | CUL2 | OGFR | LGR5 | SLC37A3 | CENPP |
| DNAJC4 | TPK1 | PER1 | MAPK4 | EML1 | KIFC2 |
| TUBA3E | FAM171A2 | XLOC_006950 | NO_MATCH_22 | TM4SF18 | TNFAIP8 |
| DDR1 | PDSS1 | XKR8 | OR2A7 | AK2 | HPF1 |
| C1D | CDK20 | ERLIN2 | HYDIN | CGB8 | CXCR6 |
| MBIP | GAGE2E | B3GALT1 | CD99L2 | RASSF8 | NANOS3 |
| ATPIF1 | MAP3K13 | NUAK2 | PIK3R4 | AGFG1 | OR10H3 |
| RBP4 | ZNF839 | AOAH | PTAFR | ZNF16 | WWP2 |
| GAB2 | EMC1 | MAPKAPK5 | NAA50 | DDX6 | CDK10 |
| S100B | TNNI3K | TIMM50 | ADAMTS5 | LRRIQ3 | SLCO1B3 |
| LCE2A | RPS26 | OXR1 | VCX3A | ADGRG6 | SLC35D2 |
| HSPA2 | LIMS2 | ZSCAN29 | RIOK3 | COL18A1-AS1 | FHL2 |
| GPR101 | TMEFF1 | MDM4 | RNF38 | LRFN5 | SLC16A10 |
| EXOC6 | XLOC_I2_015133 | HACL1 | TRIM37 | PNPLA8 | YTHDC1 |
| OR1A1 | RNF126 | BANF1 | KLHL32 | MIR17HG | CRH |
| IL11 | PHC2 | CPA1 | AMY2A | KRTAP9-6 | CKAP5 |
| MTO1 | CMAHP | IFITM1 | CHMP5 | C11orf49 | CASP8 |
| ALKBH3 | HSPA2 | AGTR1 | DNAJB14 | A2M | ADGRD2 |
| PLCB2 | HRAS | SKP2 | PGPEP1 | EPHB1 | CLDN17 |
| ZNF512B | ADORA3 | C19orf12 | IFNE | RPRM | DNAJC10 |
| MEOX1 | AVPR2 | TMEM56 | LYPD1 | HIPK3 | CCDC54 |
| PVRIG | NOD1 | TSR1 | PDZD8 | GABBR2 | PTP4A3 |
| RPL7L1 | CLEC19A | FGF19 | MS4A6A | ZFPM2 | HTR2B |
| SPICE1 | ZP1 | HINT1 | TMEM19 | TMEM102 | SUZ12 |
| CKB | LPAR5 | DGKG | DUS1L | SLC22A16 | GPR141 |
| HERC3 | ZNF41 | CCDC170 | TLR1 | KRTAP3-2 | ERGIC1 |
| NO_MATCH_35 | HLA-C | METTL4 | ZNF317 | UFSP1 | OR52B4 |
| C1orf189 | ADIG | TRIM10 | HTATSF1 | NO_MATCH_74 | SNX16 |
| PFKFB3 | INSR | HK3 | ADGRG3 | SEL1L3 | CELF6 |
| DES | PTRHD1 | PI4KA | ZNF782 | GYS2 | IFITM2 |
| SLC4A5 | RTP5 | TPPP | PLPP4 | NO_MATCH_43 | BCO1 |
| CUEDC1 | ANKHD1 | IDI1 | ZCCHC14 | HIF1A | NDUFAF7 |
| CBS | RSBN1L | ZWINT | PSMD12 | ADAM18 | PIK3CA |
| C3orf36 | SLC25A16 | XKRX | RPSA | KCNH7 | MAML3 |
| PFN4 | USO1 | TP53AIP1 | ZNF804B | WNK1 | GPR85 |
| TBXA2R | TNNI3 | ZNF140 | GYPA | AP3S2 | TAS2R50 |
| ZNF280A | WDR63 | SIPA1L3 | ERAP1 | HTR1B | UVSSA |
| FAM58A | CD59 | MAG | BMS1 | MYPOP | DIS3L2 |
| INSL3 | PTH2R | INTS7 | PIK3CB | ZNF625 | GPR62 |
| IFITS | CNOT11 | DGUOK | BLOC1S6 | GTPBP8 | MZT1 |
| TSPOAP1 | MRPL28 | PPP1R13L | MYO1C | COX10 | TROVE2 |
| NO_MATCH_6 | MXD3 | IRF5 | PIK3CA | SSX4 | TLE2 |
| KLHL20 | MCTP1 | SOST | SCCPDH | PIGB | XK |
| PEMT | CFI | CHMP2A | ELAVL4 | KANSL3 | SLC13A2 |
| RPTOR | RSAD2 | IDH3G | RPS21 | XLOC_001973 | POMC |
| CAGE1 | CACNG1 | ADGRG7 | NO_MATCH_185 | EIF4E2 | SPG11 |
| FLT3 | PALD1 | GPR156 | IDH3A | WLS | GPR87 |
| GRM2 | AGGF1 | ATF6B | DIO2 | ETNK1 | ARMCX5 |
| CENPL | KMT2E | TMEM68 | GPC1 | MYO6 | PIK3C2G |
| SHF | KANSL1L | PAQR7 | SLC47A1 | PRPSAP2 | STARD8 |
| MYO16 | TXNDC9 | PCDHGC3 | WBP4 | REG1A | PLEKHG5 |
| ATP6V1F | SLC10A1 | AK3 | PRSS35 | ENDOV | DCK |
| ADGRG6 | BCKDK | RET | ADGRB1 | KIF21B | TTBK2 |
| DNAJC10 | FAM50B | ZNF329 | SELENOK | DECR2 | HDAC5 |
| RBM12B | C9orf64 | KIF22 | CDKN2AIPNL | ZNF418 | EFCAB11 |
| DST | SMYD1 | GGA1 | ADAM21 | CDK17 | PDE10A |
| FAM71B | LOC100653061 | PGC | SLC38A11 | PPP1R14D | FNBP4 |
| FEN1 | MRPL50 | AOC3 | SERPINB1 | AK4 | ASPDH |
| MAP1LC3C | TRMU | SLC7A3 | HMGB1 | SETD5 | DLGAP5 |
| ZNF423 | 42980 | DHRS4 | CFHR3 | APBB1 | PATE2 |
| ALX1 | CYP11A1 | NENF | LINC00174 | PCDHGA9 | CALML3 |
| YIPF6 | PIK3CA | WBSCR28 | PPP1R27 | C3orf52 | CSNK1E |
| ADPRHL1 | PIK3CA | FGFR1 | PIGZ | C1orf174 | FYB |
| RORA | CDK7 | IL20RB | RAB28 | CLPSL1 | C1QTNF9 |
| SLC10A7 | RASGRF1 | PALLD | DNAJC7 | PODN | HRH3 |
| FYN | FAM135A | CALCA | GEMIN7 | STRADB | LDLR |
| OLFML2B | GDAP1L1 | TBC1D2B | XRN2 | ASAP2 | HYAL4 |
| OR8H1 | CDC34 | ACOT4 | ZNF8 | ZNF444 | TPRA1 |
| GPR146 | MET | NO_MATCH_211 | NAA15 | DDX54 | AP2A2 |
| ACTN4 | CLRN1 | LOC100653061 | TMEM14C | TAF7 | RPL14 |
| PNKD | GNG5 | HRH1 | PRRG4 | PRC1 | LAMTOR5 |
| TSC1 | WDR44 | TLR1 | TIMM17A | TRMT61B | ATF4 |
| ZNF680 | WIFI | ADGRG2 | FBXW12 | ESYT1 | GPR18 |
| RPL8 | SLC48A1 | ASPRV1 | FLT3 | CLVS1 | L3MBTL2 |
| NANOG | NEU4 | MAATS1 | CDON | DAB2IP | CLCNKB |
| PCMT1 | FRMPD2 | LOC107984056 | SORBS3 | RPGR | STYX |
| NOTCH2 | PRKACG | FCMR | PRG3 | MAOB | PSAT1 |
| RBAK | PHKA2 | Nalcn | PIWIL2 | ZNF280C | PTH2R |
| PRIM2 | MST1R | MLST8 | NAF1 | PSMB1 | DGKB |
| NR3C2 | VHLL | RAMP1 | ACAT1 | BTC | IL18 |
| ZNF529 | SLC46A1 | TAMM41 | CLK3 | DHX32 | OR52B2 |
| KRTAP12-2 | KRT13 | CDK19 | SH3GLB2 | CCR3 | ZBTB8A |
| NOL8 | ANK1 | CCDC26 | JAK1 | RBBP8 | PRKX |
| NO_MATCH_16 | BDKRB2 | CPSF6 | ID2 | INHA | CLDN18 |
| KIF2A | PDCD10 | FAM171B | GHSR | ZKSCAN5 | ACVR1B |
| APIP | SEC63 | ABL1 | IGF1R | SDF2L1 | SLC20A2 |
| FLT3 | COMT | PIAS2 | PDK1 | HERC6 | PPFIBP1 |
| CFAP43 | CLCN5 | TTK | CHEK1 | SMURF1 | NCAPH2 |
| INO80 | TPD52 | TLK2 | STK31 | ALG5 | HIST1H3D |
| ABCE1 | RPL13A | DNAJC25 | IFI16 | FBXO28 | LOC554223 |
| ARF3 | KCNMB2 | BHLHB9 | P2RY10 | NME5 | SRD5A1 |
| IGF2BP3 | CLK2 | CHRNB3 | L1TD1 | PTGER3 | SLC6A14 |
| HNRNPUL1 | C11orf87 | GRM3 | ARHGEF35 | ENPP5 | PTRF |
| DUSP26 | ZNF410 | CHCHD5 | RPLP2 | FAM134B | SAMD9L |
| USP7 | LOC102724159 | RNF5 | TAS2R9 | PXDN | CD80 |
| FAM184A | NTRK3 | CABS1 | EPS8 | COL10A1 | YEATS4 |
| FAM78B | FER1L6-AS1 | GGN | CHCHD2 | LACE1 | TRPM1 |
| OGFR | SLC22A11 | TRIM27 | CAB39 | COPG1 | DNAJC6 |
| RPP25 | HSD17B8 | RAD51B | ENTPD1 | AQP2 | BPIFA1 |
| LIF | IPO11 | IFT122 | KANK4 | FANCB | ALK |
| NTF3 | RASL10A | PPP4R4 | VIM | C11orf72 | CDK8 |
| SELENOH | EPHB4 | EXOG | EIF3G | RPL15 | SEC11A |
| KIAA1551 | SLC5A9 | MERTK | HIST1H2AI | ADGRA2 | GK |
| WDR49 | XIRP2 | IL36RN | HTR4 | CCDC97 | EXT1 |
| SOCS4 | KDELR3 | PLCB1 | RIT2 | CDK1 | SGSM3 |
| NFE2L2 | TMEM63C | EPB41L3 | ZNF83 | XPR1 | GTF2H2C |
| CSK | UBE3B | PLEKHG2 | SLC10A2 | Tia1 | SMARCD1 |
| ECD | TNK2 | LOC284009 | ZNF578 | PRPF3 | TAS2R14 |
| MAP7D2 | TTC14 | DIP2A | LDLRAD4 | RGL1 | CSNK1G1 |
| GHRL | CCDC94 | STAT3 | CYB5A | PLCD3 | TACC3 |
| SSTR2 | FLT3 | PKNOX1 | APOC2 | QPRT | DDX21 |
| ACSM5 | SEC61G | DIP2C | DSG4 | FAM107A | PHF8 |
| RTCB | ADD2 | TMEM241 | IRAK1 | PCYOX1 | H2AFB1 |
| BCKDHB | CASD1 | EFCAB6 | MCHR1 | ARAP1 | C22orf39 |
| ITPKB | OR1L3 | CD74 | PNRC2 | SEC23B | VTI1B |
| ARHGEF16 | TMPRSS3 | NISCH | DPH5 | CDC20 | MRPL1 |
| ERG | TMEM241 | KRT1 | OR51F2 | AKT1 | JAK3 |
| PHACTR4 | ELMO1 | BDKRB2 | SH3BP4 | CNFN | DIP2C |
| HAO1 | TTK | ZNF816-ZNF321P | ZNF114 | NUP37 | LIG4 |
| STK16 | ATP6V0A4 | MAPT | BRD4 | PSMC1 | ZNF10 |
| ZNF667 | MPC2 | LAGE3 | CDC27 | A1BG | ULK2 |
| USP15 | ANKRD18A | PSMD3 | IFNG | CEP70 | COG2 |
| HAUS6 | PCLAF | Mbtd1 | EPHA2 | RPP40 | PRPF4B |
| CALCOCO2 | TAS2R60 | VPS54 | RND1 | ASZ1 | VDR |
| MATN4 | PSD2 | ZNF770 | GPR132 | PHIP | RFC1 |
| SYT16 | BBS10 | GSTK1 | MC1R | TMEM248 | NXF2 |
| LOC441242 | PRPF38B | PCDHA11 | LPAR6 | PAK6 | TGM4 |
| C19orf18 | GAS8-AS1 | TAS2R31 | DCP1A | CSNK2B | ADGRF5 |
| CDC25B | CDK9 | SDHA | PIK3CA | CHEK2 | C17orf82 |
| ZNF546 | KIT | ADGRB2 | ZNF781 | ASB10 | GPRC5D |
| AK1 | RFXANK | SNX7 | PNPT1 | PRSS57 | CD274 |
| EPC1 | DLGAP5 | FOSL1 | PCMTD2 | PLRG1 | SEMA4A |
| AP5B1 | COASY | CSNK1D | RBM26 | SMARCAL1 | DTYMK |
| HEY2 | CENPA | NO_MATCH_256 | GBA | ARAP3 | RYBP |
| KDM8 | PROKR2 | ABL1 | SLC7A13 | LCA5 | HENMT1 |
| CDV3 | VPS33A | PHF21B | COPB1 | ELP2 | MAPK9 |
| ZNF211 | RNF130 | RAPGEF4 | ZNF461 | SLC35F3 | SMIM12 |
| CRYGB | TCAF2 | SLC7A2 | IL16 | WDR66 | C12orf10 |
| ZFP2 | P3H2 | NOP16 | MAN1C1 | RPL5 | ZNF511 |
| RPS6KA3 | PRKAG2 | ZMYM4 | ADGRF5 | TNFSF12 | DIRAS3 |
| EDA2R | COPS5 | SCNN1B | AGPAT3 | HSPD1 | FLT3 |
| SCYL2 | DPP4 | KCTD3 | NO_MATCH_96 | UPK2 | PDE7A |
| MINK1 | OMG | KANSL1L | BSND | SLC6A15 | NCR3LG1 |
| COL4A3BP | EGLN3 | MAPK10 | RBM14 | FAM134B | SCGB2A2 |
| CPSF6 | WBSCR17 | CENPX | OR6C65 | MET | FBLN2 |
| BRPF1 | HDAC3 | LOC642249 | PLD1 | NIPA2 | SLC13A4 |
| FCHO2 | PRPF31 | MAP4K3 | PSG9 | CUL5 | HIPK3 |
| Dock10 | KLB | MED24 | SLCO1A2 | METTL21A | NT5E |
| OCRL | CSN3 | LINGO1 | DPPA4 | RBPJ | LRRK1 |
| ZNHIT2 | IL13 | ALPK1 | Nrbp2 | PSG8 | SLC6A7 |
| APH1A | GEMIN2 | OR56B1 | CCNC | SMARCC2 | ZC3H12D |
| RHOA | FAM3A | HSPA9 | TMEM144 | ALPI | RNASEH2C |
| BCL2L12 | GAS8 | C11orf71 | ARHGAP27 | EFHC2 | PMPCA |
| OASL | VPS33B | MOGAT3 | STK33 | NSUN5 | DHX36 |
| UBA2 | MAP10 | CCDC62 | NR2C1 | EYS | SNX22 |
| H2AFB3 | TAAR6 | RAB1A | HIPK4 | FNDC1 | GMEB1 |
| SATB2 | DMRTA1 | SCYL2 | DDX23 | LIG3 | MAGI1 |
| ATP6V1C2 | CTLA4 | C1QTNF9B | RRH | HSPA8 | LRRTM3 |
| CLK3 | APRT | THAP12 | RASL10B | PDIA3 | MIOS |
| MT1X | HLA-DPA1 | ELK1 | TRMT5 | PIWIL4 | IRS1 |
| SMC1B | USP54 | TLK2 | EIF3C | KRTDAP | LENG1 |
| SPINK13 | GLUL | SLC9A3R2 | RBMS2 | ZNF266 | NO_MATCH_279 |
| CALM2 | SH3BGRL2 | ZBTB21 | KIAA1324L | BATF2 | SESN2 |
| LMX1B | LINC00597 | KRT4 | DRAM1 | MCM2 | RAPGEF6 |
| TPST1 | ECSIT | ZNF175 | GAS2L1 | AK6 | CHRNA3 |
| PLCG2 | PHKB | DPH7 | MCM7 | OR8D4 | LCLAT1 |
| OR52L1 | ADGRL4 | TRIM24 | SOCS5 | NDUFAF2 | ZNF503 |
| C7orf33 | PLCG2 | KRTCAP2 | LATS2 | CCKBR | LTN1 |
| RFC1 | NR0B2 | MILR1 | DCLRE1A | NEK11 | BFSP1 |
| TPCN2 | WDCP | SRFBP1 | OXER1 | AZU1 | APEX2 |
| NME6 | GRM5 | C21orf58 | MASTL | SSH2 | LHX4 |
| AMBRA1 | LPAR5 | CYB5R2 | FLT4 | TCIRG1 | KIAA0895 |
| ATXN7L1 | NO_MATCH_28 | ZNF556 | LCE3D | KRTAP9-3 | HAVCR2 |
| KIF2B | CCNC | L2HGDH | CIB2 | ABI3BP | FGGY |
| FGF21 | Spcs2 | EPHA3 | CBWD1 | ZMIZ2 | EPHA2 |
| PXDNL | FZD6 | USP8 | IDE | CCDC66 | ZBED4 |
| TAS2R45 | SLC16A4 | DNASE1L3 | GRIA1 | TGFBR2 | HMGA1 |
| HTR2A | ZNF484 | NO_MATCH_67 | BCAT2 | TAS2R4 | SEZ6L2 |
| PDE4DIP | DENND2A | GAS6 | PPIB | LYZ | PZP |
| TMEM38B | E2F8 | FAM149B1 | App | GRM8 | ZNF131 |
| MCCC1 | ADGRF1 | ABRACL | MAS1L | OXER1 | OR4D6 |
| ZNF608 | CDH7 | VN1R2 | CADPS | NO_MATCH_8 | HIST2H2BE |
| FAP | SLC35F6 | SHKBP1 | NO_MATCH_123 | GAN | MAP3K14 |
| PPCS | LINC01554 | EIF2AK3 | NO_MATCH_125 | COBL | RIPK4 |
| FBXL4 | VCX2 | SNX14 | CDC7 | ZMYM1 | LOC254896 |
| GPRC6A | MIS18BP1 | FRMD8 | ADGRL4 | TKTL2 | PHOSPHO2 |
| ZNF85 | CKS2 | DAPK1 | SGSM2 | CCDC148 | STKLD1 |
| ARG2 | TRDN | IL12RB2 | DDR2 | PRPS2 | UGT2B7 |
| PROKR2 | ZNF268 | LGR5 | BMPR2 | QARS | MYEOV |
| AP3B2 | BRINP3 | FBXO11 | CSTF2T | PTGER1 | DMTF1 |
| ARMCX2 | MYLK | NO_MATCH_240 | ZNF253 | SHOC2 | PKDCC |
| COL4A3BP | MET | TNXB | RASA1 | XLOC_013840 | LOC100506127 |
| RGS1 | DPYSL3 | NMRK1 | PSMB9 | RUNX1 | ULK4 |
| DDX27 | ELF3 | SIX2 | RPF2 | TAS2R4 | GRIA2 |
| ZNF638 | PPP1R13B | S100A7 | ARID5A | FAM229B | MIA2 |
| LOC81691 | ZNF761 | STAT3 | ETFBKMT | FBXO8 | EPM2A |
| TTLL4 | FLT3 | PPARA | L3MBTL4 | C8orf22 | RDH11 |
| SLC28A2 | BRCC3 | TFCP2 | GABRP | RSL24D1 | SLC43A3 |
| ELP5 | ZNF418 | FAM120A | CYLC1 | MEF2D | DDX20 |
| VHL | ITPKA | TCF25 | AKR1C1 | C18orf21 | DKK3 |
| PSMC4 | CD93 | COX4I1 | SLC30A1 | BOLA1 | ATF7IP |
| NO_MATCH_37 | SIX4 | ADSSL1 | APLF | GPR162 | ALOX15B |
| RBP2 | PART1 | CDKL2 | MAN2B2 | FAXDC2 | SLC9A6 |
| KLC2 | UBE2D2 | MUM1L1 | CXCR2 | CYSLTR2 | ERBB4 |
| SLC25A52 | MADD | TTK | GSE1 | ZNF816-ZNF321P | NOA1 |
| KCNH6 | CLK3 | BRCA1 | CEP95 | ARR3 | RSPH10B |
| PITPNA | C11orf1 | TONSL | RBKS | MRPL52 | ADGRB2 |
| C1orf50 | EPHX2 | USP4 | CSF1 | NO_MATCH_238 | NID2 |
| ABL1 | HNRNPC | SLC22A17 | HIP1 | UFL1 | PPY |
| SLC26A2 | ZFYVE16 | AQP7 | ANXA6 | FASTKD2 | RAB9B |
| TSSK1B | LTBP2 | CSNK1G2 | FGF3 | TMEM213 | Zfp317 |
| ITSN2 | ZNF35 | ZFP37 | STH | ZNF343 | CORO2A |
| TASP1 | EPHA7 | MAPRE2 | NPFFR1 | SP6 | EVL |
| TLK1 | TSPAN2 | MYCBPAP | IFNGR1 | KIT | WNT9B |
| MAP4K3 | MAPK14 | VARS | TSPAN3 | MATN2 | LPP |
| UCK2 | E2F7 | RPS14 | ITIH5 | PCGF3 | C11orf54 |
| C6orf10 | COQ7 | TMEM161B | GCLC | ERVV-1 | PLAT |
| ENTPD4 | ITPRIP | GPC3 | RERGL | DNMT3A | GALNT8 |
| FAM160B2 | HNRNPCL1 | FAM71F2 | ZNF311 | MCM2 | GLIS3 |
| ALDH1A3 | PCDHA4 | DIS3L | MEP1A | MAGEA9 | C7orf31 |
| DEDD | MCM6 | NUMB | OR10AG1 | PARP6 | ICA1 |
| PITRM1 | SNRPA1 | TRIM3 | DPYSL3 | FPR2 | GPR101 |
| NBPF15 | TAP2 | DLC1 | CDAN1 | C9 | GSG2 |
| CLCA4 | RPS18 | TWF2 | KIF3A | FAAH2 | HFM1 |
| ZNF569 | EIF2AK3 | MRPL44 | PAEP | PLK4 | TMEM255A |
| CCDC178 | LRGUK | TMEM92 | USP14 | RAB2B | LGR5 |
| OR6B2 | SLC20A1 | SRC | CC2D1B | TAS2R7 | SDHD |
| JMJD1C-AS1 | PLCD4 | WBP11 | PC | KIF3C | TMEM39A |
| RAB3D | C21orf91 | PJA1 | ASMTL | ANKRD20A3 | LNPEP |
| PEA15 | EPHA7 | ACVR2A | FBXL20 | DSTYK | LYSMD1 |
| FAM72B | RAB3IP | NDUFV2 | SRPK1 | OR3A4P | NUDT13 |
| LPAR1 | BANP | NAE1 | NO_MATCH_73 | STX5 | AVIL |
| RNASE4 | TAS2R13 | SLC22A11 | IQCA1 | MINK1 | CALN1 |
| TAF9 | FGFR3 | HIST1H4L | RGS3 | TMEM63A | ZDHHC6 |
| COX7A2L | Olfr981 | SNRNP25 | RHEB | TREML2 | ZNF107 |
| VIT | ADGRA1 | RSPH10B | UBE3B | VPS35 | PIP5K1B |
| TMC7 | STARD13 | PDE6B | POLI | NCKIPSD | MRGPRX1 |
| RNF111 | APEH | ADRB3 | NDUFA9 | PCK1 | C9orf78 |
| SUPV3L1 | GALR2 | NO_MATCH_50 | STRIP1 | SNRPB | NFATC1 |
| LEPROT | AASDH | XLOC_I2_013393 | ADSSL1 | TAS2R7 | PCDHGB5 |
| LIAS | FAM102A | KLB | XRCC6 | NFE2L3 | FAN1 |
| TRIM11 | RHOA | LBR | IFT27 | KCNIP3 | ARPC4-TTLL3 |
| BMPR1B | ACYP2 | RXFP1 | UCP3 | EDEM3 | CEP162 |
| HEXIM1 | INTS10 | TMEM182 | S100A12 | PROKR1 | ZSCAN12 |
| CARD6 | GRAP2 | PTTG2 | ORM2 | NLN | NPY1R |
| C9orf3 | NO_MATCH_163 | TTLL4 | CALCRL | IQSEC1 | EPB41L1 |
| PIK3CA | PIK3CA | USP3 | CFL1 | ANXA4 | NDUFAF1 |
| SLC52A2 | LRRCC1 | ERAL1 | PARN | DGUOK | OR2J2 |
| SATB1 | PCDHGB1 | IQGAP3 | ZPBP2 | ADCY2 | C7 |
| ALG11 | COA6 | EBAG9 | FRMD5 | ARHGAP5 | BHLHA15 |
| ADCK5 | PLCG2 | DR1 | LINC00242 | RPS29 | MST1R |
| GTF2IRD2B | WDR1 | DDX11 | OR8G2 | ZNF44 | HDAC6 |
| CPA4 | PTK7 | HCST | EXTL3 | SUMO1 | KRT86 |
| EGLN3 | RBM12 | PIGA | DDRGK1 | POLR2J | GPR85 |
| GPAT4 | PRDM1 | NTPCR | NADK2 | DDX59 | DUSP28 |
| KPNA5 | NME6 | MCM9 | KIR2DL4 | OGDHL | CARS |
| DCAF4 | MYBPH | ANGPTL1 | OXSR1 | ATF7 | BGLAP |
| JAKMIP1 | TEKT1 | SLC5A7 | LINC00636 | ZNF532 | ATP1B1 |
| FAM160B1 | SARDH | ZNF85 | KLHL10 | LMBR1L | VSTM1 |
| DYNC2LI1 | PAPOLA | ACAT2 | BAG6 | SERTAD4 | ZNF267 |
| UTP23 | TCF4 | VPS11 | ZNF227 | LRCH2 | VAV1 |
| TLK2 | ARMC3 | GHITM | EHD2 | SETDB2 | BRD4 |
| NDN | TM9SF3 | PAK4 | COASY | BANK1 | MLN |
| CLCN4 | ARMC1 | KRTAP9-8 | TPTE2 | ITM2C | NSUN6 |
| OR5T1 | SETX | CNOT10 | SPHKAP | KIF12 | MTM1 |
| GPR139 | KRTAP10-1 | PCDHA6 | CLK1 | LYN | ITGAE |
| FPR2 | BMP2K | ZFP41 | MCM3 | HOXD3 | XYLB |
| MAPKAP1 | OR14J1 | OR8D1 | ZC3H12B | CASC4 | SMPX |
| MAP2K6 | FAM111B | DARS | MARK4 | G2E3 | XIAP |
| CDK16 | BLID | RBM17 | TMEM144 | SLC38A2 | SGK2 |
| NO_MATCH_54 | TMEM185A | PRNT | LINC01567 | TTC8 | WDR55 |
| AKTIP | Churc1 | ASGR2 | PDE4B | MAST1 | XPO1 |
| XPO7 | MIER1 | BBS7 | SLC52A3 | ZNF184 | JUP |
| PNPLA6 | VPS53 | MAP2K6 | RPS6KA6 | SLC41A1 | ZNF138 |
| CXCL10 | VAMP3 | LY9 | HMBS | WDR41 | COQ9 |
| TLK2 | XPO5 | RPS6KA2 | NARF | STK10 | ZNF43 |
| SLC30A6 | Thap12 | KCTD9 | ZNF420 | ARHGEF10L | TANGO2 |
| GYPB | KCNJ12 | PTPRA | ZNF12 | GNG12 | SNX6 |
| PPP2R3B | GRK6 | MAP3K13 | ADCY4 | CLEC4G | HIST1H4I |
| OR2B3 | ZNF77 | LOC102724428 | LCA5L | OR52W1 | TRMT1L |
| ATP10D | OR1J1 | GSK3A | MAP4K1 | FKBP4 | FSIP1 |
| OARD1 | COQ3 | GIMAP8 | RPS6KA1 | ADGRG7 | ADGRB3 |
| CKAP2L | ANAPC7 | DPP3 | 42799 | ZNF846 | MORN5 |
| EARS2 | ARHGAP44 | GPR141 | RAB3GAP1 | CHL1 | NARFL |
| TNC | CRHR2 | TLR7 | PRPH | ARL8A | ANKRD39 |
| RUNX1-IT1 | NO_MATCH_145 | EXOC1 | LRSAM1 | ARMC7 | UGT2A3 |
| KMO | PLEKHN1 | BBOX1 | RERG | ZNF518A | ABHD12B |
| SRBD1 | MT3 | LPAR4 | HVCN1 | SKIV2L2 | ALMS1P1 |
| SYPL1 | IGFBP7 | PTPN6 | SERINC1 | UCK1 | SLC2A13 |
| ACADS | NME1-NME2 | ECHDC3 | NEK4 | NO_MATCH_84 | CFAP65 |
| FNDC7 | KPNA2 | USP26 | POGK | ACO1 | ZNF678 |
| BUB1 | PTK7 | TSPAN3 | SUMO3 | CHGB | NO_MATCH_17 |
| SLC2A4 | ABL1 | CDHR2 | TAS2R43 | FZD2 | LRRN1 |
| GFPT2 | FBXO42 | FAM151B | PCNX2 | GRM7 | TSR2 |
| AK8 | TAS2R50 | USP6NL | CDC42SE1 | DHFR2 | DYRK2 |
| HIST3H3 | WNK1 | MSH5 | RFX6 | SSTR4 | CTLA4 |
| DNAJB12 | PCDHA12 | B4GAT1 | TNFSF14 | AKR1D1 | EML3 |
| GRK2 | PHLDB1 | UIMC1 | CNIH2 | LGR6 | PUS10 |
| KBTBD2 | STXBP3 | P2RY11 | EPS15 | FAM234B | RIOK2 |
| SAMM50 | GFM2 | SERPINA9 | TAS2R60 | PDCD4 | RUFY2 |
| ACOT12 | HDAC8 | EPHB3 | EGFLAM | PKN3 | CYP17A1 |
| PIGO | MAP7 | CASK | HSD17B14 | KIT | ZNF555 |
| CLDN7 | Med1 | NEK3 | EPS15L1 | RRS1 | AFG3L2 |
| ZNF572 | BIN3 | NO_MATCH_149 | RBAKDN | KCNIP1 | EXOC2 |
| CYP3A43 | DUSP1 | IFIH1 | SIRT2 | COPA | ATP6V0A2 |
| C2orf66 | LOC102724984 | WNK4 | CHRM2 | ACVR2A | SLC22A1 |
| POLR2B | JAK3 | EIF4E2 | ZNF300 | OR2K2 | MRM1 |
| RET | GJD2 | FTSJ1 | UHMK1 | C8orf59 | ATRIP |
| NO_MATCH_33 | RAD21 | ZNF718 | PNMA3 | ANO4 | SLC5A2 |
| PLA2G3 | PIK3R2 | PTK2B | BMP1 | LRRTM4 | SLC52A1 |
| CACHD1 | MAP3K6 | F2RL2 | ERBB4 | PDGFRA | ZNF141 |
| SCGB1D4 | CHD1L | FLT4 | ZNF628 | TMEM187 | KLK1 |
| UGDH | NO_MATCH_105 | GBP1 | NO_MATCH_4 | GPBP1 | GRK2 |
| ATP6V0A1 | ZNF37A | PADI4 | XLOC_010017 | HS6ST1 | RNASEL |
| PCDHGA2 | NKRF | DDR1 | PRDX5 | XLOC_00H64 | EIF4A3 |
| PHF14 | GPR149 | GRM4 | NADK | PLEKHG3 | HIST2H2AC |
| DENND5A | PRMT6 | RSBN1L | AMPD2 | USP53 | CDK18 |
| RBBP4 | BAG5 | YARS | PKP2 | FER | MTMR7 |
| DGKG | CSRP1 | WDR36 | WARS2-IT1 | FSTL1 | SKP2 |
| MZF1 | ANKHD1 | ZNF596 | BBS4 | FAM3C | PTPN14 |
| DDR1 | C3orf58 | TBL1XR1 | ZNF701 | MAP3K14 | INSR |
| MATK | C14orf180 | PHACTR3 | HLA-DPB1 | GTF2IRD2B | FAM120A |
| NHLRC3 | PLS3 | TAS2R8 | ZDHHC13 | CHRAC1 | SLC32A1 |
| FSHR | ACVR1C | NO_MATCH_71 | MASTL | CDK1 | SYDE1 |
| PAM16 | EDNRB | DPF3 | RCBTB2 | LINC01553 | TGM3 |
| HSD17B4 | GLRX3 | ZNF276 | MRPL19 | TSEN2 | ADK |
| VANGL1 | ESCO1 | ACSBG2 | VIPR1 | ACTR1B | GPS2 |
| RTCB | SLC2A3 | MYDGF | RPS15A | PPM1F | SCRN3 |
| FAM60A | BICD2 | TMEM97 | DCLK2 | RPL27 | TVP23B |
| SUN2 | STXBP1 | ZNF79 | DRD2 | RTKN | POLD1 |
| GPR62 | TMEM135 | Alkbh3 | SUMF2 | HCAR3 | HBS1L |
| NO_MATCH_225 | FAM105A | DAZ1 | CMA1 | LCLAT1 | PFKM |
| CYBB | CCDC189 | PRSS30P | LINC00521 | MAPK6 | STYK1 |
| SIK3 | SCLY | POT1 | ACOX2 | TMEM156 | HIPK4 |
| IST1 | SLC16A11 | RNF213 | HELLS | RAD51C | MDP1 |
| APLP1 | ZFP28 | HNRNPUL1 | UNC93B1 | ZNF92 | GK |
| CNOT4 | SLC12A8 | ARHGAP8 | MRPS35 | MYLK3 | OR6A2 |
| EXOC3L4 | TMEM120B | DYNC1I1 | HK3 | PDIA3 | PAAF1 |
| PRDM7 | YIPF3 | ERO1A | ANGPTL7 | UHRF1BP1L | GPR17 |
| SPRR3 | CLGN | ZBED5 | COLEC12 | RPS6KA4 | SLC12A4 |
| C20orf144 | OR51Q1 | PHKG1 | EML1 | CYB5R1 | TTLL12 |
| NO_MATCH_2 | SMO | OS9 | OS9 | CPB2 | OR52B2 |
| KSR1 | CNNM4 | FAM126A | COG7 | XLOC_I2_004844 | HRH4 |
| ORAI1 | FABP3 | SLC44A3 | NO_MATCH_191 | FAM32A | LINC00173 |
| PIK3CG | NPBWR2 | RET | ESYT1 | NANP | ZNF485 |
| IDH2 | KCTD15 | P2RX5 | GLRA1 | CTSE | ADAM2 |
| F2RL1 | MECOM | CCDC140 | RPP14 | CLDN19 | TRPC3 |
| AFM | PIK3C3 | PSMC3 | AGPAT3 | TAS2R14 | TPRKB |
| ZMYM3 | CAMKK1 | CCDC151 | ERN1 | AAMDC | RPH3AL |
| MTMR9 | DUOXA2 | TP53RK | TPM3 | MAST1 | GLB1 |
| UGT3A1 | ATP4B | EPHB6 | TTBK2 | METTL22 | SLC9A9 |
| UCK2 | EIF5B | ZCCHC12 | FLT3 | TRAF6 | ATP5A1 |
| STAT6 | HLCS | CDK8 | SLC2A3 | TP63 | BLVRA |
| LRGUK | NEDD9 | RHBDF2 | OR9Q1 | KLHDC4 | KAZN |
| C11orf98 | RIN1 | ATP6V1D | CS | DISC1 | TUFT1 |
| FCF1 | GEN1 | AFAP1L1 | HAVCR1 | HS3ST2 | AMPH |
| ADGRF3 | SIL1 | AMACR | GPCPD1 | SPG21 | MAPK15 |
| EPHB1 | TPRA1 | CARD11 | SOWAHB | RPA1 | FANCD2 |
| MXD1 | GLP2R | INHBC | METAP1D | LEO1 | TP53INP1 |
| OR4X2 | CAMKK1 | SBNO1 | PRKCQ | OR5D18 | DYNC1H1 |
| PAPSS1 | ATP5F1 | LIPM | ULK3 | DMD | SPECC1L |
| IMPA1 | GTSF1L | PAK3 | OSGEPL1 | MAP10 | PRAG1 |
| MAP3K19 | MRPL4 | XLOC_008362 | PDCD6IP | EXOC3L4 | STK3 |
| ADCK1 | RNF6 | ADAM32 | XLOC_I2_000394 | TAS2R5 | RFC4 |
| PDGFRL | SNRK | RABGGTB | KCNG3 | FRMD5 | MC3R |
| GLTSCR1L | DNM1L | POU5F2 | PCDHA8 | CPSF2 | KIAA1324 |
| RAB31 | KLHL26 | NME2 | ODF2L | NO_MATCH_3 | FASTKD2 |
| NXT2 | C6orf58 | VRK2 | OSM | BRD8 | MST1R |
| BLK | TCOF1 | SLC35F5 | RHNO1 | PTEN | APBA2 |
| PRKX | AK1 | BRS3 | ALOX5 | SLCO6A1 | ITPKB |
| RARS | HBG2 | TOMM40 | NO_MATCH_61 | HECTD2 | SEMA6D |
| SLC6A2 | OXTR | RNF34 | PPWD1 | NFE2L1 | SYTL5 |
| MARK3 | CYP11B1 | CKS2 | HIKESHI | ZNF433 | MAP2K4 |
| CAMKMT | CNMD | CMTM4 | PBX4 | ABL2 | CAPN9 |
| BUB1B | PPM1A | PLA2G4C | TMEM110 | CES1 | ZDHHC20 |
| GALNT14 | S100A13 | NUP210 | IRAK4 | SLC14A2 | OR8B12 |
| TEC | DTWD1 | MERTK | TPGS2 | CBLB | NXPE3 |
| RAB41 | THADA | TFAM | TCHHL1 | RAB34 | HSD17B7 |
| OR4K2 | BMP7 | FAM19A2 | PCDH20 | MAP4K5 | TRMT1 |
| PWWP2B | MRE11 | PPP3R1 | TCTE1 | OPTN | NOX1 |
| ZNF660 | PNLIP | THEMIS2 | CNTN2 | RECK | PTH2R |
| CNGA4 | DDX27 | FAM208B | CERS4 | TUBD1 | HTR1E |
| RPAP3 | PAK5 | CFHR3 | IQCB1 | TFE3 | ADGRG7 |
| SCYL2 | IGSF8 | PIP | CALR3 | SAMD4A | NO_MATCH_223 |
| LRRTM4 | ZNF3 | TRAPPC1 | ECT2 | KIFC3 | EPHA1 |
| PAGE4 | NDUFA2 | TPD52L3 | PIP5K1B | RET | MCEMP1 |
| PDGFRB | TTC30B | UGT1A4 | PRKAA1 | B3GALT2 | 42989 |
| MINK1 | TMPRSS13 | PCDHGA6 | HTN3 | ALG10B | ARG1 |
| NR1H2 | LY6G6F | NFATC3 | ZNF302 | DFNA5 | SNCAIP |
| TAS2R14 | CSNK2B | AGTR1 | TMEM2 | PPP1R12A | SPATA13 |
| TDP1 | NDUFA4 | TAS2R13 | KIAA1524 | SRSF4 | WDR93 |
| PANK4 | DGKG | ZNF331 | VPS39 | LRRC37B | TAS2R50 |
| PDPK1 | PRPF40B | TYK2 | MRPL28 | RABEP1 | COL5A1 |
| SEC23IP | SRPX2 | PPDPF | EME1 | SLC2A1 | MAT2B |
| XPO5 | EXT2 | FOXN4 | CERK | IL2RB | ACADVL |
| ADRA2B | GPR31 | BUB1 | TDRD9 | GPR6 | ARMCX3 |
| RPS6KA1 | TAOK3 | DHX38 | AQP4 | PCSK9 | DYRK1B |
| DRAM2 | TAS2R31 | ACVR1B | CKS1B | C1orf186 | MTERF1 |
| CDK9 | PRPF6 | C9orf170 | SAP30BP | PUM3 | PACSIN1 |
| ZFYVE21 | PCK2 | SLC5A7 | SPINK2 | ADGRG5 | EIF2S2 |
| MSN | DGKZ | CCR8 | PER3 | ERBB2 | MAPK8IP2 |
| ADGRE3 | RTN2 | FGA | MTMR10 | EFHB | LILRB1 |
| SLC22A7 | R3HDML | MAPK1 | CCDC141 | WFIKKN1 | OTUB2 |
| CWC22 | ADGRE1 | MC2R | COPS7B | PCDHGB4 | SENP6 |
| CLK2 | EXO1 | CHID1 | LY96 | DEK | ATP9B |
| C1orf101 | MYLK4 | KCNK7 | GPSM1 | TCEA1 | DHX40 |
| NPHP1 | MARK3 | MORC3 | LRCH3 | PFKFB1 | OR5B12 |
| ATRNL1 | IL17F | FRK | CRTC1 | ATM | NO_MATCH_280 |
| KIF7 | POMGNT2 | ADIG | MIA2 | HORMAD2 | PFKL |
| HINT3 | DGKK | MX1 | OR2D3 | VEZT | ZNF260 |
| TFIP11 | VASN | NCAPD3 | GPR149 | RCHY1 | DEFB4A |
| DARS2 | KLHL2 | GJA4 | NRROS | PDGFRA | RPUSD3 |
| SIK2 | PHKA1 | ACKR3 | SQLE | Xkr6 | ABCF3 |
| INSRR | SHC4 | GALNT7 | CHD2 | DZIP1 | MAP2K2 |
| SS18 | PCDHA12 | RING1 | PRR16 | RAB27B | TIMP3 |
| ROCK2 | LIMK2 | ANKS3 | EPHA3 | AGPAT2 | CPNE8 |
| ADORA3 | BSCL2 | AP1G2 | PFKP | PAK4 | SART3 |
| PTK2B | RBM19 | SPDYA | MAST3 | TLE1 | PTK2 |
| C19orf70 | ANGPT1 | ERC2-IT1 | KATNAL1 | SLC27A1 | SLC22A14 |
| CLK4 | DLX4 | BMX | MCM2 | USP2 | VPS25 |
| BCL2L14 | MAST2 | PIAS4 | APP | NPBWR2 | ZNF677 |
| MRGPRX4 | CDK4 | PPP1R13B | USP1 | ACVRL1 | ZNF786 |
| LOC55338 | RNPC3 | HIF1A | MAP4K5 | RBMX2 | RHBDD3 |
| ASPSCR1 | DEFB104A | ACAA1 | ALG14 | ERMN | PYGB |
| PGLS | MMP19 | DTNBP1 | NECTIN3 | SERPINB3 | RBM6 |
| GLB1L3 | MRPL3 | ZCCHC5 | ABL1 | EEF1A2 | CCDC89 |
| YY1AP1 | RXFP3 | MSL2 | PRKACG | BRAF | ADGRF2 |
| SYK | MAPK7 | MCM5 | SLC5A11 | MGAT3 | SCFD2 |
| HSPD1 | PPP1CC | FCRL5 | SSRP1 | TXNDC16 | PAGE3 |
| CH507-42P11.6 | DHX57 | ACSL5 | OR52N5 | CLVS2 | IGHMBP2 |
| GAS2L3 | UBL4A | ELL | TRAPPC12 | SLC15A1 | PNMA2 |
| CCDC40 | UBE4A | HTR1E | IGFBP1 | SNAPIN | CBR4 |
| FAM234B | GPR183 | HDX | MDM1 | KLHL12 | ZNF513 |
| TUBGCP3 | IFIT3 | OR51E2 | PIPSL | ANXA2 | PRAF2 |
| FAM200A | FAM30A | SLC28A2 | CAPN1 | PRPF19 | PUS7L |
| RPL35 | FAAP100 | SORCS1 | CYLC2 | IMMP2L | WDR46 |
| ATAD2 | NSUN5 | SYK | NEK4 | LZTS2 | YY1AP1 |
| INSIG2 | SORCS3 | RHBDL2 | CD200R1 | SMTN | SP100 |
| TPRG1 | CHST15 | PPP1R16A | LARS2 | ADH1C | FAM98A |
| ELF4 | ACKR3 | GPR39 | ADGRE5 | SERPINE2 | CYP19A1 |
| NAA30 | C6 | ABCC3 | KIAA0391 | OR2AK2 | ZNF670 |
| MAGEB6 | ABL1 | ZNF124 | GRM6 | EXOC3 | HCFC2 |
| SPANXC | PPP3CA | RALBP1 | ERGIC3 | AXDND1 | GRIP1 |
| GPR101 | OGFR | KLC3 | ANKEF1 | PLCL2 | TRPV5 |
| TAS2R43 | MTDH | ERBB4 | CALM3 | LINC00574 | TKTL1 |
| ITGA5 | MYOD1 | CASQ1 | SPATA6L | FZD3 | PPFIBP2 |
| SLC4A3 | HSP90AA1 | TYK2 | TEX10 | TRPS1 | FGFR3 |
| POLK | KRT2 | CDY2A | PDK3 | CEP57L1 | ADGRG1 |
| ACKR4 | CLK2 | HIST1H2BH | PAPSS1 | MAN2C1 | GLIPR1L2 |
| GRPEL2 | NO_MATCH_216 | PTK2B | KIF2C | OR52K2 | GTF2A2 |
| NO_MATCH_176 | RABEP2 | NHLRC4 | MAPKAPK5 | PJA2 | MTFP1 |
| HCAR2 | GIP | NUSAP1 | LINC01312 | ARHGAP44 | JAGN1 |
| MYL2 | DPP8 | PSME4 | CDH23 | VWCE | PCDHA2 |
| TAOK3 | NMI | TOR1B | SMURF1 | TNIP1 | GAREM1 |
| SAMD3 | KLK11 | ST5 | OR10W1 | BZW1 | DDX39A |
| STK33 | NUDT16 | GPR75 | ADCK1 | ZNF678 | ERN1 |
| HCK | ZNF112 | SERTAD1 | Nalcn | PFKP | RPS6KB1 |
| E2F3 | DYRK2 | NET1 | ZNF791 | FKBP9 | RNF40 |
| TBK1 | SNRK | NLRC4 | GLP2R | PRPF4 | DTX3L |
| ZNHIT1 | GSTA1 | UBE3C | MMP2 | POMT1 | N4BP2L2 |
| CHRM1 | ZNF23 | ZBTB48 | DHTKD1 | CLK2 | CTXN1 |
| NTRK3 | KRTAP5-6 | PRKG1 | MTM1 | NO_MATCH_231 | LCK |
| TAS2R31 | ATP6V0E2 | ZNF136 | SORBS1 | SPAG8 | ARHGAP17 |
| FOXM1 | EPB41L2 | DDX55 | SF3A3 | WDR91 | FTSJ3 |
| PLPP4 | LOC105371493 | PFKL | LOXL2 | PFKFB3 | SSTR1 |
| GPSM1 | FKBP2 | RBM10 | ZFP2 | P4HA3 | AFP |
| PTPRH | TSKS | MAPT | GOLM1 | FZD5 | LUM |
| DHX38 | FAM217A | FPR3 | ZNF426 | ZNF177 | CFHR3 |
| ISOC1 | NMUR1 | PKN3 | SPG20 | ACVR1 | IFI27L1 |
| STK16 | SCYL1 | CTPS2 | DUSP6 | IPPK | DDX18 |
| MAP3K6 | RIT1 | PRIMPOL | OR5C1 | PCDHAC2 | HSPA12B |
| STRBP | LILRA1 | DNAJB6 | CTNND1 | ELAC2 | FPGT-TNNI3K |
| KCNJ3 | OXGR1 | RINT1 | BCL2L11 | ATP6V0E1 | RIC8B |
| MAX | LMLN | ZNF658 | MAP2K5 | TRIM5 | BVES |
| SSTR1 | CYP2C18 | DDX19B | 42985 | ADCY2 | SIK2 |
| MAT2B | CHRM3 | PPM1E | LRFN3 | RPS6KA1 | PDGFRB |
| RAB3B | TUBA1B | SPEG | SPPL3 | BMX | PFKL |
| NGRN | TRIM15 | EPHB1 | PANK2 | MEIOC | TRAFD1 |
| NR3C1 | IL13RA2 | PTPN12 | TGFBR1 | TMPRSS11B | NECAB3 |
| ELMOD3 | LTB4R2 | ZNF326 | IREB2 | USH1C | SLC30A4 |
| DYNC1I1 | SH3GL3 | TRPC1 | LOXL4 | LYZL6 | GPR153 |
| LOC102724334 | NKAIN1 | SNAP47 | USP15 | NO_MATCH_234 | RPS11 |
| MCRIP2 | AP2B1 | S100PBP | MAGEB3 | OAS2 | RPL23AP7 |
| BMX | KLHL23 | PRKCD | IFIT1 | FMOD | NTRK3 |
| SAMHD1 | NPSR1 | PANK4 | PTPN3 | OR51M1 | SARS |
| SPDYE17 | PCCA | GPR101 | FGFR3 | PRKC1 | GRHL1 |
| PLOD3 | AGT | SLC35F2 | CENPC | TACR1 | PHKB |
| Zfp777 | ZNF586 | UTP3 | TGFBR3 | TBC1D10A | HFM1 |
| MAP4K2 | BCAP31 | SLC27A2 | C17orf105 | ROR1 | STX5 |
| SLC2A2 | ADAMTS15 | CPEB1 | BCO1 | NOXO1 | ADRB2 |
| UNC45B | MYL3 | BRD4 | DPH1 | AFTPH | CALM2 |
| PHEX | LRRC6 | PTPRN2 | MAPT | GPR75 | EMP1 |
| TK1 | SPP1 | SULF1 | PTK7 | ZNF219 | PFKM |
| IL12RB2 | PYGL | CLK3 | CCDC162P | TMEM182 | SLC52A2 |
| GNPDA2 | ATAD3A | TGFBI | PRF1 | PLCL1 | HSP90AA1 |
| MORC2 | FGD5 | SIM2 | ENTPD5 | TRIB2 | GTSE1 |
| REC8 | HNMT | GRIA3 | Clpb | TXNL1 | WWOX |
| CKB | GTF2E2 | SERPIND1 | WDR34 | DDX18 | TSTD2 |
| GNAO1 | DNA2 | RPL34 | CAMK2B | C8A | C2orf15 |
| CD36 | NEXN | NO_MATCH_38 | RASGRP3 | TLK2 | VILL |
| CLOCK | NO_MATCH_246 | NCAPH2 | DDX41 | FAM49B | NKD1 |
| POLR2D | Osbpl6 | EPHA1 | CTSS | GNAI1 | SMYD4 |
| ASIC1 | MFSD14C | PPP1R16B | ST8SIA5 | UGP2 | TPP1 |
| RFX4 | SIK2 | ARHGEF40 | TLE3 | TMEM260 | MOXD1 |
| ZFP3 | JKAMP | CDC20 | RTCB | DPP7 | JAK3 |
| FAM111A | ZNF214 | DSC2 | CBR4 | MRGPRX2 | NO_MATCH_196 |
| ERBB2 | CASC1 | Tmprss7 | IRAK4 | MAS1 | CEP63 |
| PRB4 | TECPR2 | KIZ | ETV6 | PIGX | DEFB125 |
| GJA1 | XRN2 | ADRA1B | RGL3 | MLNR | HDAC7 |
| NTRK2 | NMRK1 | KIF3C | CKAP4 | FLT4 | PIK3CB |
| PEX6 | LLPH | FAM13C | DEFA3 | PRAME | PHF20L1 |
| NO_MATCH_89 | DSE | TAS2R16 | SPTY2D1 | E2F8 | ELL3 |
| VLDLR | NO_MATCH_244 | HIST1H2BA | METTL17 | FHOD1 | CWC27 |
| SARNP | LASP1 | SMARCAD1 | NTRK2 | CACNB2 | SLC28A3 |
| LOC102724023 | USP11 | LARP1 | PARD6B | TESK2 | TP53BP2 |
| URB1-AS1 | NPHP3 | PTER | XLOC_I2_004594 | EIF4B | ZNF775 |
| PFKM | EEF1D | IDI1 | NPY2R | CHGB | OTC |
| BIRC2 | NR4A2 | APOBEC3B | RDH11 | PLB1 | LILRA3 |
| TBC1D2 | PPP3R2 | ABCF2 | FRMPD2 | SLC35A3 | NPR2 |
| CORO7 | MAPK7 | SLFN12 | OR10H1 | VCAM1 | PPP4R2 |
| IFNL2 | SYNGR4 | OPTN | FZD7 | CCL20 | CSNK2B |
| MRPL33 | RUNX3 | LFNG | STK24 | MIA2 | TOMM70 |
| LCP2 | DUS4L | ACADM | PIK3CB | CRTC2 | NO_MATCH_275 |
| TGFBI | C7orf49 | LARP1 | CSNK1D | FZD7 | PDXK |
| ZFP64 | CYP8B1 | CD6 | TCF25 | NEXN | UGT3A2 |
| XAGE3 | FGR | ADRB1 | AXL | CAPN6 | FRMD3 |
| OSGEPL1 | DTYMK | PRKG2 | FASTKD3 | FER | LARP7 |
| DERL1 | B4GALT6 | SKIL | BBX | DRC3 | CHMP1B |
| NO_MATCH_189 | WAC | MYBL1 | ZNF645 | SNX9 | KCTD3 |
| MEF2A | ODF1 | HIST1H2BG | PGAP2 | DEFB106A | QPRT |
| ANXA10 | FGGY | ZNF394 | FAM45A | LAG3 | SIRT1 |
| OPRL1 | FZD4 | RNF20 | MPEG1 | KCTD12 | PIK3CG |
| NAT10 | PKN1 | RAD51AP1 | LGR6 | CCNC | KIAA2013 |
| ACAP3 | HIST1H3H | PAK5 | ELOA2 | ART1 | KCNRG |
| SP4 | C10orf67 | ZNF34 | ETS2 | NO_MATCH_62 | PPP1R42 |
| XLOC_I2_004840 | LOC107986912 | NO_MATCH_121 | IGF1R | CBLC | NO_MATCH_210 |
| KRBA1 | KDR | ADRB3 | ARID3A | TRPC4AP | GSG2 |
| DCST1 | CNBD1 | BPI | CLK3 | CARD9 | TMEM5 |
| ULK4 | ABCF3 | GPR139 | DRD1 | FAM161A | PPP2CB |
| ATP1B2 | FAM114A1 | FBXW7 | AMOT | GNRHR | MUSK |
| SLC7A8 | FGFR2 | SELENOT | SLC35A4 | SLITRK4 | MRRF |
| PITPNM3 | LYN | PEX6 | DPF2 | EYA4 | PSMD14 |
| KRT24 | ATP5B | TCF25 | ADPGK | NO_MATCH_27 | CEP19 |
| FXR2 | CKB | FH | TAS2R39 | TRIM6-TRIM34 | SMAD1 |
| SLC7A9 | GNE | L3MBTL1 | PLA2G12B | CHRM3 | PPARGC1B |
| TTLL7 | GPBP1 | NEK8 | NPM1 | MAP2K5 | MYD88 |
| AP2M1 | DPP6 | ZFY | LRIG1 | PTK2B | RSPH1 |
| NO_MATCH_162 | EZH2 | GCLC | WWTR1 | SLC44A5 | USP2 |
| WASF1 | RMND5A | ARPP19 | PINK1 | PPP3CB | PIK3R1 |
| PRKG1 | GPR68 | GTSE1 | OCLN | TLR8 | RPL6 |
| CGA | P2RY4 | GOLPH3L | TMEM252 | TNIP1 | TNP2 |
| TAS2R43 | ICOS | PPP1R21 | GTF3C3 | CFAP97 | PRRT3 |
| PRAM1 | LINCO1711 | TPH2 | ZNF350 | TMEM108 | AP3D1 |
| TAS2R20 | HP | CASR | SRPK1 | MCOLN2 | PPIL3 |
| DNAAF2 | ZMYND11 | STAT4 | CORO6 | MYO10 | ALCAM |
| TM7SF2 | DNAH6 | ZKSCAN1 | ALAS2 | TLK2 | CDKN3 |
| DNAJC3 | PYGB | HNRNPH1 | SLC1A1 | ZNF554 | EIF2S2 |
| KCNE4 | FAM19A1 | NSMF | IFNAR1 | ZCCHC14 | CAMKV |
| CEP290 | CDC45 | PRIM1 | MAP3K20 | INPP4A | SLC23A3 |
| TMEM229B | HNRNPF | CNOT10 | ATL1 | WDR47 | PIGY |
| ADRA2B | DET1 | MMACHC | ADCK2 | TGOLN2 | PRPF4 |
| TBL3 | RPS6 | F2RL1 | DDHD2 | GRIN1 | C10orf25 |
| DIRAS3 | THOC1 | GPM6A | CHRM5 | COL20A1 | PTGS2 |
| ALDH2 | SLC5A11 | NUAK1 | EXOSC3 | TEX30 | C11orf24 |
| SNX14 | CAMK2A | GSDMB | ZNF213 | OR8I2 | MGEA5 |
| UBE3C | PAK4 | PCDHGA5 | CCT5 | USP46 | RELT |
| TTC9B | CADPS | ANKLE2 | RNF213 | SLC28A3 | SYNPO2L |
| CXCL2 | AZIN1 | BEST1 | CCR8 | ANKRD54 | NO_MATCH_184 |
| SPRR1B | SYK | WDR19 | CDADC1 | EYA2 | MAP3K20 |
| UQCC1 | 42801 | USP33 | ZNF783 | LMOD3 | ZAR1L |
| CEP76 | PRKAA1 | RCN3 | TRAF3IP1 | SYTL4 | MTHFR |
| ILDR1 | CAPN13 | KSR2 | LRRC18 | SLC47A1 | RPEL1 |
| PNLIP | TNFRSF1A | HNRNPA1 | GALNT9 | KCNAB3 | EGLN1 |
| KNOP1 | NTRK2 | TPPP | WDR76 | BMP5 | ADGRG2 |
| XLOC_I2_006010 | NAB2 | RFK | CDYL2 | FAR1 | DIP2A |
| FBXO30 | SLC22A14 | PTPN9 | ZNF567 | SMAGP | LGR6 |
| POF1B | LOC107987253 | CIRBP-AS1 | IGFBP5 | MEPE | CDC14A |
| FUK | TINAGL1 | LUM | OSBPL11 | PKMYT1 | SYNCRIP |
| FBXL16 | RASGRP1 | ZNF18 | MAX | SPON1 | HECTD3 |
| SRSF5 | MISP | UBXN1 | THUMPD3 | TBCEL | TSPO |
| IFT57 | BMPR1B | ATP5H | PAX8 | CSNK2A1 | USP30 |
| SRPK2 | KLHDC4 | DPYSL4 | ZNF800 | NTRK1 | SLC28A1 |
| EPHB2 | AQP10 | NPBWR1 | OR2L2 | HADHB | C9orf142 |
| ANKHD1 | ZRSR2 | ZGPAT | ADGRL4 | JADE2 | PGM2L1 |
| FYCO1 | EDNRB | LIPF | LUC7L | DNAJC19 | FBXO5 |
| NOP2 | G3BP2 | SLC3A2 | SERPINA10 | P2RX1 | CHST12 |
| NPR3 | SOX5 | ZNF34 | MAP2K1 | TTC19 | CSNK1G2 |
| RAF1 | TP53BP2 | IMPG1 | HOXA9 | PES1 | GADL1 |
| TNFRSF1A | COMP | SUMO1 | SPHK1 | CREB3L1 | SUPT20H |
| ZNF485 | OGDH | OR11A1 | RUNDC3A | LPAR4 | ATP6V1B2 |
| TKFC | EID1 | PDE3A | CRYGS | RXFP1 | DRD4 |
| SLC12A1 | SELL | PDZD11 | ICAM3 | ACSM3 | CAMK2B |
| PPP1R7 | ANKRD10 | PALM2 | NO_MATCH_133 | LGALS3BP | TBK1 |
| SPARC | ZNF530 | LAMTOR3 | MFGE8 | BRS3 | COG5 |
| ZSCAN21 | TNFSF14 | ARRB1 | ZNF224 | RXRA | RARB |
| DCAF8L2 | ZNF33A | SHTN1 | TRPC4 | WDR20 | MRGPRX4 |
| EEF2K | MINK1 | SLC2A9 | GPR87 | GPT2 | MPND |
| ARHGEF28 | SMAD5 | ATP6V0E2-AS1 | PCDH9 | REP15 | OAT |
| TAS2R40 | MOAP1 | G6PD | DACT2 | GK | GMPR |
| MTR | TIGD4 | TMEM243 | PAX7 | ZNF449 | ALPK1 |
| HSPA1L | SIRT6 | ZNF829 | CPEB1 | TTLL7 | PI4KAP2 |
| AXL | POLH | YIPF1 | SYCE1 | ELAC1 | ZNF254 |
| YTHDF3 | COL8A1 | BMP2K | REN | Myt1 | EIF2AK3 |
| RTKN | SCGN | NO_MATCH_264 | SOX3 | RBM28 | RYK |
| GSC | MRPL43 | PA2G4 | GATM | SPNS1 | NO_MATCH_15 |
| MAP3K7 | SYNJ2BP | OR4F16 | TTC6 | SFSWAP | DCBLD1 |
| NR0B1 | ST7 | 42990 | DAPK1 | CABP1 | ANKRD13D |
| RPL35 | POP5 | GCK | FYN | SRP54 | PDE1C |
| EOGT | AGRP | BRD8 | ERH | PIK3CD | NO_MATCH_220 |
| HIPK2 | APBB3 | ADSS | FAM86C1 | TOR1AIP2 | MAPK9 |
| SULF1 | TNPO2 | NPR3 | C20orf27 | RIOK2 | SACM1L |
| OMD | SRC | NO_MATCH_172 | MPP3 | ZFP82 | GRHL3 |
| FSCN3 | PEAK1 | C12orf60 | BTK | PDE1B | SLC25A21 |
| Rassf3 | PDE9A | AGTR2 | ZNF532 | CHUK | OR6B2 |
| NELFA | C12orf40 | GRIN3A | NOX5 | LDHC | NFYC |
| NEK5 | RARG | CAPN11 | ADGRG2 | TAAR2 | JAKMIP2 |
| PRPS2 | PPIL4 | C2orf73 | ZNF655 | DRD4 | CXCL8 |
| FAM83B | POMT2 | PKN2 | NLN | ADAMTS12 | ZIK1 |
| NELFE | NR1H3 | DYRK3 | SSX2IP | ZNF607 | UMPS |
| NO_MATCH_91 | PLK2 | RASGRP2 | TGFBR1 | TDRKH | TULP2 |
| HPGDS | AQP9 | DYNC1I1 | PPM1D | EML3 | RNF219 |
| ADORA1 | MIEF2 | STOM | NDUFAF5 | HK2 | ZNF544 |
| CERS2 | WNT5A | ARHGAP8 | ZNF2O2 | MOXD1 | TRPC5 |
| CCDC38 | XLOC_005923 | CNR2 | SLCO3A1 | WNK4 | CHRM1 |
| MRPS18B | TRPM8 | TRAM1 | OSBPL5 | CDK16 | EIF2AK2 |
| S1PR5 | DNASE2B | RANGRF | DEFB119 | PSKH2 | FBXO25 |
| PTEN | HLA-DPB2 | SF3B2 | PPP2R2D | DUT | PSMD4 |
| ARMC5 | LIMK1 | PDZK1 | PIK3R5 | TRMT6 | GGT7 |
| ZNF404 | BST2 | HMGCR | NOL9 | FUT2 | CFHR5 |
| BMPR2 | HNF4A | LOC101060386 | NME1 | CLHC1 | CCDC186 |
| GZMA | ARFGAP3 | FUK | ANKRD44 | S1PR3 | APPL2 |
| MLPH | PRIM2 | RPL21 | UBA5 | AMMECR1 | SCYL3 |
| GUCY1B3 | OCLM | CHRM4 | VILL | MAGEC2 | CSNK1G2 |
| SENP2 | CLP1 | KLK15 | LAIR2 | POLR3E | TOR1AIP1 |
| CD1B | FAM209A | RPL22L1 | CLINT1 | ZNF345 | DMC1 |
| ARHGEF7 | KCND2 | PCDHGC4 | ALDH6A1 | TAF1B | RSRC1 |
| ZNF333 | XPNPEP1 | PHYHIP | ACAA2 | ZNF558 | NO_MATCH_69 |
| PTPRO | KIAA1143 | P2RY13 | TOM1 | AGBL5 | CYP4A11 |
| HCRTR2 | RASA3 | GPR82 | Acss2 | RIOK3 | NO_MATCH_271 |
| BEX1 | TNFRSF1A | TSSK1B | CCR4 | LCK | SERPINB6 |
| SPHK2 | PLEKHO1 | MOXD1 | PPP1R18 | SYK | CDC5L |
| TACC3 | PRKCI | DAPK3 | AK9 | PDPK1 | TRHDE |
| FIGNL1 | MAP3K9 | WDR74 | ISL1 | NPR2 | PITPNA |
| GNE | FOXRED1 | PNPLA6 | POLR1B | SSBP1 | CAPN5 |
| MIIP | NARS | RBKS | MGST3 | GNA12 | RPS6KA3 |
| GRK3 | PAPSS1 | EIF2AK2 | PRSS1 | ADGRG7 | ESRRB |
| NO_MATCH_134 | GAB2 | NO_MATCH_215 | DNHD1 | 42988 | ASXL2 |
| STK11 | GRM8 | ZC3HAV1 | IFNAR2 | ADH4 | SPATA22 |
| CCHCR1 | PPP1R8 | DIP2A | NAGK | COQ6 | UBE3A |
| MAPK8 | SRPK2 | Usp48 | POLD4 | ZNF253 | ZNF334 |
| CHRM5 | TCEA2 | SIK3 | ALKBH5 | AGTR2 | ZCCHC8 |
| ACCS | SH3BP5L | IGF2BP3 | RGS3 | NLRP4 | PRKCA |
| CDSN | JAK3 | EIF3L | LPL | WDTC1 | NPR2 |
| HDHD2 | SRP54 | UBAP1 | SBSN | ZNF622 | GRK2 |
| LARP1B | XLOC-013189 | PCM1 | MRFAP1 | NO_MATCH_195 | FAM161B |
| RGS13 | KIF5C | WDR1 | POTEB3 | SRGAP1 | UNC45B |
| FBLIM1 | STK3 | PAK4 | CCNL1 | ARV1 | TRAF3 |
| GBP6 | BRD2 | KXD1 | LATS1 | C3orf30 | MAPKAPK3 |
| BICDL2 | DTNB | GNAS | GRM2 | NUMBL | KLC1 |
| TTI1 | GGT5 | KRT23 | SSH1 | RLF | DKC1 |
| PRKX | FXR2 | MAP3K14 | NSD3 | ARNT2 | NO_MATCH_108 |
| CYB5R4 | POC5 | HSPA9 | ICOS | PPP1R15A | IRAK4 |
| C3orf62 | RPL7 | MDM4 | C11orf57 | C3AR1 | ATP6V1B2 |
| SGPL1 | DNAJC7 | LIPG | IRF8 | NO_MATCH_126 | FASN |
| PLK2 | L3MBTL3 | UTS2 | CCDC136 | TAAR1 | FAM134C |
| GPR85 | TAS2R42 | MAPK14 | HSPA1B | CMTR2 | DDX21 |
| VPS45 | MATK | GGTLC1 | ZNF695 | ODAM | ATG3 |
| TAOK3 | TPR | CPT1B | ZC3H11A | AP4B1 | CYP4F11 |
| DBN1 | TRIM41 | C6orf48 | TNK1 | PLK2 | QRICH1 |
| TRPV3 | HES1 | ZNF146 | DAZ4 | CD86 | HOXC10 |
| MPP5 | FSHR | ALPP | INSR | PRC1 | ZNF618 |
| RBM47 | PLXNA3 | GCC1 | SSX2 | TRIP4 | CRIPT |
| ZNF439 | SLC2A11 | ZCCHC9 | USP37 | CRY1 | DCBLD2 |
| MMP15 | DBNL | MAP4K2 | GCSH | DDX11 | KLHDC2 |
| RIC8A | DMPK | PCSK9 | LASP1 | SLC5A7 | TSN |
| ZMYND8 | PSMD13 | ZDHHC17 | HIKESHI | EPHA5 | ZNF467 |
| SYNDIG1 | ENTPD3 | ECT2L | XLOC_013689 | PAX3 | PDCD1 |
| CLK3 | CLK3 | MORN2 | TAS2R19 | RIOK2 | MTPN |
| UTP11 | GALK2 | ACTR1B | AKT3 | ALPP | EBF3 |
| NO_MATCH_51 | SLC39A1 | NXF1 | IGFN1 | TIMM29 | MFSD13A |
| TAS2R19 | KLHL11 | CERK | SLC27A4 | PSMC5 | TM4SF19 |
| ADRA2A | CRISP2 | IL1R2 | PCDHGB2 | SLC22A5 | CDC25A |
| ZBTB8OS | APP | PFKFB1 | GPR156 | HIST1H2BC | AOC2 |
| PLK3 | TESK2 | SLC4A1AP | RHOBTB1 | USE1 | PLVAP |
| STAU2 | BLZF1 | CLMN | EPHA2 | LRRC49 | LCN2 |
| SELENOI | MRPS17 | ZBTB24 | HCAR3 | AGPAT1 | ELOVL5 |
| TGFA | C19orf57 | FABP4 | FCGR3B | CAPZA2 | LRRC14 |
| ZNF577 | PPIP5K1 | EIF2S1 | ZNF273 | POPDC2 | CELA2B |
| GLUD1 | PTPDC1 | POLD3 | FAM71B | BMPR2 | 42982 |
| PPP4R1L | EEF2KMT | KCNK15 | ABCA9 | FUZ | SLC35A5 |
| EEF2 | C19orf44 | FBXO24 | ULK2 | SEMA3C | CTC1 |
| HMMR | TFDP1 | GLOD4 | ZBTB14 | LLGL2 | NR1D2 |
| NUP37 | SPHK2 | MAPK6 | CDIPT | PCK2 | YAP1 |
| MPZL2 | ASIC4 | PRICKLE3 | CPNE4 | BTBD10 | DNAJC14 |
| CDK18 | LOC153684 | TM9SF1 | MSI2 | MAPK8IP1 | EDNRA |
| CLK3 | MS4A10 | CA5A | STK38L | PREPL | MDM1 |
| RIPK1 | OSGEP | HSPA6 | XLOC_014105 | ERBB3 | MANF |
| KLC3 | DRAXIN | NPSR1 | YTHDF1 | ZFAND4 | GNS |
| PMM2 | STAG3 | KLHL36 | KRTAP22-1 | SUN5 | XLOC_010072 |
| BAALC-AS2 | TIGD5 | OR5M11 | SLC26A2 | TMEM177 | PASD1 |
| RSPH4A | SLC2A2 | NTRK3 | DDRGK1 | GLP2R | PRPS2 |
| LRRN3 | KCNS3 | OPCML | MAP4K2 | SRPK1 | CDC23 |
| VIP | EML4 | RASSF6 | OR8B8 | ACSF2 | SH3D19 |
| MC2R | KRT6C | RBM5 | C17orf47 | EXOSC3 | NEK6 |
| MPHOSPH6 | RNF182 | KRTAP8-1 | CHRM4 | PDZRN4 | SLC52A2 |
| SLC35A5 | DCTN2 | ZBTB12 | GPR25 | PNLDC1 | KRT7 |
| STAT5A | ORM1 | KIF3C | PPBPP2 | DLG5 | DSCR8 |
| AVPR2 | PRPF39 | SERBP1 | TLK1 | PTCD3 | UBE3D |
| PAK5 | PLK1 | MFAP1 | PPOX | CNOT3 | TCF7 |
| RNF139 | PRKAG2 | CELF5 | SDCCAG3 | KCNA6 | NEDD1 |
| DMTN | SNX32 | IMMT | VPS50 | BLK | RHBDL2 |
| THUMPD2 | CLK4 | NCOA4 | MAPK10 | OPRL1 | REXO4 |
| SLC35A5 | DPH2 | HPGD | MAP4K4 | ABCD3 | UMPS |
| IL18RAP | PRKAA2 | SEC22C | CFAP57 | ZNF764 | BAZ2B |
| HDGF | MAP3K8 | LINC00477 | BTN3A2 | ATG16L2 | TMCC1 |
| TRIM65 | CCDC81 | GAPDH | ILVBL | ZNF645 | GPD2 |
| KIF12 | XPO5 | PMPCB | BMPR1A | HIGD1A | ZFYVE26 |
| PCSK5 | BIRC8 | TYW1B | Aak1 | FLT3 | CDKL3 |
| STK25 | FADS2 | EPSTI1 | U2AF1 | CMIP | CXorf21 |
| HK1 | ATP5A1 | BMPR1A | RABGAP1L | ZNF415 | NO_MATCH_132 |
| C3AR1 | RWDD1 | ZFYVE1 | PCK1 | PRKCZ | OPN5 |
| DLG1 | CLASP2 | LRRC19 | CCDC112 | MLX | ZNF76 |
| ESR1 | SPRED1 | VN1R10P | IQCC | CALCR | CISH |
| FAM213A | NAP1L1 | GPR85 | ZNF837 | ATOH1 | CTCF |
| PLTP | EP400 | WDR76 | CAPN8 | EPB41L1 | ZNF75A |
| UBAC1 | C11orf58 | DONSON | COMMD4 | ABCB6 | HTR3A |
| PHF20 | KIF9 | MRGPRX2 | NPY5R | MAP2K7 | CLIP3 |
| PFKL | SLIRP | SLC28A3 | CDC123 | SMCR8 | IP6K3 |
| TBX22 | YES1 | LRRK2 | PAIP2 | RSL24D1 | CNDP1 |
| FGG | RNF31 | PALM | SPDL1 | NTRK3 | HOXA1 |
| USP10 | ZBTB39 | GALNT1 | MSTO1 | TBX15 | ATPAF2 |
| HOXC9 | GPBP1L1 | XLOC_I2_006797 | FOXO3 | PHF21A | Ube2o |
| AHCYL2 | DHX30 | ST6GALNAC2 | SRD5A2 | GPR61 | MESP2 |
| SYK | ARHGAP17 | CHPT1 | PCOLCE2 | CYTH3 | NO_MATCH_112 |
| ADAMTS1 | ZNF599 | AK9 | NCF2 | SNW1 | NMD3 |
| FADS1 | BRPF3 | C22orf34 | SLC41A2 | OR13A1 | TRIM44 |
| CKM | IFNL3 | MCM7 | RHOH | FZD10 | ADGRE2 |
| CNBP | POLE2 | RBM48 | PPP2R2C | PICALM | FAM43A |
| FANCC | PDK1 | ZFP91 | OR7C1 | 42980 | GANAB |
| NO_MATCH_124 | KIAA1324 | IL13RA1 | TUFT1 | POLR2M | ZNF257 |
| MCMDC2 | RARS2 | CRTC1 | ARHGAP22 | MAJIN | GFOD1 |
| SLC2A13 | NDUFB11 | OVOL2 | TCF4 | KIAA1257 | RGS22 |
| ACVR2A | PRMT1 | RBM46 | PRKCQ | SEMG1 | ASS1 |
| OXCT1 | CNOT9 | HDAC1 | EFTUD2 | PDK2 | TCF7L2 |
| DAB2 | HIST1H1C | IKBKE | ILF3 | NEK10 | INTS4 |
| POU5F1 | IKZF3 | ZC2HC1A | NCOA5 | GAD1 | ACTBL2 |
| EXOSC10 | RYK | CCNG1 | STYK1 | NOP10 | CDK5R1 |
| MVP | KIAA0319 | ARHGEF25 | MAP2K3 | GPAM | PSKH2 |
| ZNF225 | NADK | HTR2B | GPR139 | OGFOD1 | CMKLR1 |
| ATXN3 | PPIL2 | MKNK1 | KNG1 | RPS6KA3 | MAP3K7 |
| SPERT | SASS6 | SLC2A2 | PAMR1 | XLOC_001272 | MRPS15 |
| GRAMD1C | SUFU | ZNF613 | LYSMD3 | CHRFAM7A | LMO2 |
| MINA | SREK1IP1 | SSX2IP | MYOC | TLR2 | GRM7 |
| PIK3R5 | ENTPD2 | KIT | GAA | PAK3 | AASDHPPT |
| CKMT1A | EYA1 | PRKCB | ORC6 | PRDM4 | NO_MATCH_154 |
| NO_MATCH_102 | HEATR1 | MLLT3 | TOB1 | CBX6 | FGFR2 |
| VDAC2 | PDCD6IP | MAPK11 | FZD10 | OSBPL3 | MAP2K2 |
| TSPAN1 | HDAC6 | DNAJB3 | LRTM1 | SOCS6 | SPRED2 |
| RFX3 | DUSP19 | KCNN2 | SORD | MYO3B | HADHA |
| SMIM8 | KIAA0319L | LSR | SCARB1 | JAM3 | RSBN1L |
| SEMA3E | XLOC_I2_006166 | PRAME | FAM114A2 | NUDT16L1 | BTF3 |
| PPP4R2 | LHCGR | CCL22 | EPHA7 | GCLC | RNF135 |
| SNX25 | SFPQ | ZGPAT | SSR2 | 42984 | TOR1A |
| SLC26A11 | CYP3A5 | TEPP | RPS3A | BMP10 | NPR2 |
| TYW3 | FOXA1 | ART3 | TAC4 | ASGR1 | C9orf43 |
| PSAT1 | NT5C3A | UTP4 | GPR78 | SULF2 | TMEM39A |
| PNN | NSMCE1 | IFT81 | FAM150A | GPKOW | MAPK4 |
| MARVELD3 | DPH6 | MCF2L2 | RIOK1 | TOMM34 | B3GNT5 |
| CAPS2 | CFB | HLA-DRB1 | FYN | HIST1H1A | LSS |
| TFG | HIF1A | FBLN7 | ARHGAP28 | TRMT1 | GTF2H1 |
| ADPGK | PLPP3 | DHRS9 | VAT1L | TSEN54 | GPR35 |
| CYP20A1 | SLC26A2 | SULF2 | LRRFIP1 | SDHD | AMHR2 |
| NO_MATCH_103 | EEF2K | IFI27 | OR8G5 | KIFC2 | GNA14 |
| GLTSCR2 | CLSTN2 | GPBP1L1 | CYP39A1 | HTR3E | SPIN1 |
| RLN3 | RHBDF1 | KLHL31 | PHB | NMB | MEIOB |
| TSNAXIP1 | NRP1 | CTDSP2 | SPESP1 | COLGALT2 | FAM83F |
| JOSD2 | PDGFRL | SPRTN | TMEM42 | EPS15L1 | GSDMC |
| JAK1 | FGF7 | GMCL1 | SERPINH1 | OPRL1 | SNX7 |
| RBBP5 | PYGB | STXBP3 | AKIRIN2 | PIN1 | USP44 |
| PDXK | AKT1 | ARRDC3 | PMF1-BGLAP | INPP5E | NO_MATCH_19 |
| LALBA | HS1BP3 | RSU1 | MME | DGKE | DPYSL5 |
| ICAM4 | COPS4 | OR2H1 | CCR3 | TCF7L1 | SAMD4B |
| STAU1 | HSPA1B | CDC73 | PPIL2 | BOLL | TBXA2R |
| SEC61A1 | FGFR1OP | CDK11B | TMEM14B | FGFR3 | BABAM1 |
| KIFC2 | LRRC37B | ZNF57 | B4GALT4 | CDKL1 | URI1 |
| ZCCHC10 | PAK2 | GPR26 | RANBP10 | SUCLG2 | HLA-F |
| TRO | ARNTL | SLC18A2 | ADORA2B | EFHC1 | ADSS |
| NR1I2 | GPR151 | CST11 | DGCR8 | TMEM45A | RRAGC |
| METTL25 | DEFB121 | CDC7 | ALG10 | CCDC91 | STRADA |
| ACSM1 | NDUFAB1 | SLC17A1 | ABAT | IKBKB | PLEKHG6 |
| OTUD3 | TRAM1L1 | SNU13 | HOOK1 | PSG5 | INPP5D |
| FASTKD5 | BTK | MYNN | PPP2R3C | STRADA | NSUN2 |
| GPR119 | SYN3 | CPPED1 | KLHL36 | FGF9 | EPHX1 |
| NR3C1 | CA2 | ELMOD1 | RACK1 | SYTL5 | PPP1R2P9 |
| ZBED1 | PCDHB5 | NO_MATCH_174 | SLC26A9 | SP1 | XLOC_009028 |
| XLOC_003546 | ERO1B | ZNF561 | MTHFR | HSD17B10 | Olfr981 |
| MAS1 | ZNF366 | CLIC3 | AIFM3 | ETAA1 | TRAT1 |
| DDR1 | NO_MATCH_262 | TNK1 | OR4C12 | TNFRSF14 | ACVR2B |
| NO_MATCH_36 | TCEAL5 | GSPT2 | GAPDH | HTR7 | PUF60 |
| ACPP | MCPH1 | GPC4 | NUMB | APOBR | RBM4 |
| GPBAR1 | PAPSS2 | TXLNB | CXCL11 | MB21D1 | HDGFRP2 |
| WSCD1 | RHOBTB2 | NO_MATCH_175 | VPS28 | PIGP | MCM10 |
| EIF2S2 | IRX2 | NUAK2 | DCAF8 | CX3CR1 | MAP3K20 |
| KRAS | REC8 | ETFA | PDXDC1 | CCR8 | ANGEL2 |
| ADA | SULT1A3 | MKX | CCR5 | SULF2 | KLHL4 |
| OR51B4 | TNNT1 | SRPK3 | ARMC10 | CCNI | MAST3 |
| C7orf25 | PGD | IFT22 | MOXD1 | PUF60 | ADGRE5 |
| PTER | CDKAL1 | AP3M2 | ZNF232 | EFCAB7 | RHOD |
| JADE3 | BMP4 | SIRPA | ZNF264 | COX7A2 | GNG13 |
| ZC3H12A | CIDEA | RNMT | CHKB | Rbp1 | KRTAP4-12 |
| TYRO3 | FCGR3B | BMP2K | SCAMP1 | NO_MATCH_21 | NR2C2 |
| KAT5 | SRRT | DKC1 | TIMP1 | GABRA4 | SCYL3 |
| COMT | PCGF5 | MTMR14 | OR8G5 | SCPEP1 | TXNDC5 |
| FFAR1 | CHRNA7 | THRA | HCRTR1 | KIF12 | TEPSIN |
| SYCE3 | DLAT | ALAS1 | NGDN | DEDD2 | NPSR1 |
| MAOA | MAP3K11 | NOL4 | QSOX1 | PDHA1 | KRAS |
| ANKDD1A | ENKD1 | DNAJA1 | CCBE1 | ZNF792 | MITD1 |
| UPP1 | USP5 | BLMH | SCYL1 | KIF2C | JSRP1 |
| PRMT6 | RANGAP1 | SERPINA7 | MAGEF1 | GNL3L | PAPSS2 |
| DNAH17 | CHGA | Csrp2bp | TRAF5 | KPNA3 | CSK |
| CCM2 | EDIL3 | PCGF1 | PPWD1 | COQ8B | COPS5 |
| KCTD13 | FTH1P3 | C17orf80 | WLS | USP54 | HSD17B11 |
| BMPR1B | DGKA | MAP3K5 | TSPAN6 | P4HA3 | PROKR2 |
| DDX53 | IL2RG | S1PR2 | MIER1 | TCAIM | DCLRE1C |
| NT5DC2 | NBN | DHX57 | BAIAP2 | DDX39A | RGMB |
| CALY | FAM46D | NO_MATCH_56 | MPZ | HCK | TMPRSS11A |
| PHF1 | IRAK4 | ENTHD1 | VRK1 | BRINP2 | ARHGEF10 |
| TSPAN18 | SMO | VIPR2 | UQCRC1 | TAGAP | MRGPRE |
| ZNF454 | SMG6 | S100A2 | TFR2 | GNAI2 | PLAUR |
| FAM124A | USP12 | RCVRN | NO_MATCH_117 | SFN | VIPR2 |
| GABBR2 | NSMAF | VMP1 | TIGIT | RHPN1 | SNTG2 |
| ABTB1 | NDUFS1 | GPATCH2 | INTS5 | ZRSR2 | FOXB1 |
| ROR2 | SCP2 | ITIH3 | SEMA4C | ADGRG7 | RABL6 |
| ZNF266 | USP27X-AS1 | PRR3 | FUT8 | PCDHGA7 | ATCAY |
| PDE1B | DNASE1L2 | WSCD2 | AURKB | SNAPC5 | GSTK1 |
| MAS1L | SLC38A6 | DBR1 | TNK2 | SERPINA12 | SLC25A13 |
| ACVR2B | FES | RGS6 | RNF144A | GPNMB | PLEKHA8 |
| ABL1 | HOXB13 | ATXN1 | GPR101 | SLC2A5 | CERCAM |
| LMOD1 | PEPD | EPHA7 | STK38L | SMG5 | HNF4A |
| SIRT1 | AP1M2 | GPR161 | DIDO1 | GPR4 | DRAM1 |
| LDHAL6B | EIF2AK1 | OSBPL5 | CLEC2B | PPARA | VEGFA |
| SPOPL | MAPKAPK2 | GDI2 | GPR6 | PRKACB | MID2 |
| NO_MATCH_239 | MATN4 | RASEF | PARP15 | MS4A15 | ARSF |
| WAPL | TBC1D22A | NECTIN3 | GPR157 | ATG4C | OR13G1 |
| ORAOV1 | CFH | BTD | TMEM14B | ZNF286A | CDH8 |
| SLC43A2 | PATH | HTR1A | TAB1 | PRH1 | ACTG1P17 |
| TLR3 | NPRL2 | NO_MATCH_92 | KRT31 | GLMN | KLRC4 |
| LIPG | BOD1 | MSL3 | PTK7 | ADCY2 | NEK8 |
| AGFG1 | TK2 | PNPO | NOP58 | CYTIP | OR2G6 |
| SSX2 | GNPNAT1 | HUNK | TRAF3IP1 | USP48 | SDF4 |
| FRMD5 | PRKACB | CLEC4E | C6orf141 | TRIM21 | ATP6V0B |
| MAGEE1 | TUBE1 | FSCN2 | ASB9 | STK11 | LINC01561 |
| DNAJC11 | HIST2H2BF | ST7L | SAR1B | DCTN6 | RNF214 |
| NOL12 | HDHD2 | BATF | EYA2 | ATP13A4 | FAM81A |
| MST1R | NEK11 | CLK4 | TOR1AIP2 | CDH17 | CNNM2 |
| CBARP | PRKD1 | PNLIPRP3 | SDHB | GPER1 | NPR2 |
| PLEKHA8 | NR2C2 | SH3KBP1 | HAS3 | GDF9 | ALDH1A1 |
| ABCB8 | ADRA2A | ETHE1 | IGF2BP2 | TCP10L2 | NO_MATCH_18 |
| RPS6KA5 | LMNA | FAM83A | CDKL3 | EYA3 | GPR89B |
| DNAJC7 | ACBD4 | WWP1 | CTTN | FGF17 | HLA-DOB |
| MAPK13 | XLOC_I2_014804 | C1orf162 | NAT10 | ZC3HC1 | UGCG |
| RTN2 | VWA9 | NR2C2 | APH1A | CNST | HSPA9 |
| PHAX | CLK2 | RPA4 | RMND5A | ZNF341 | AHCYL1 |
| URGCP | SLC22A1 | PSME1 | SLC46A3 | NECTIN1 | BTN3A3 |
| NAA38 | KCNK2 | COQ5 | SMAD3 | OR2B6 | ADGRE3 |
| MYLIP | SOX14 | RPL8 | PLSCR3 | PIGG | OR51B5 |
| BCAM | PHKG2 | ALG1L | NTRK3 | CLEC14A | ANAPC15 |
| CMTM2 | LARP4B | SERF2 | CPNE9 | RNF32 | WIPI1 |
| Hkdc1 | ZSWIM2 | SLC35G1 | GPAT2 | NPBWR2 | BZW2 |
| NO_MATCH_192 | XLOC_I2_004840 | TBC1D23 | HIP1R | STK32C | ZDHHC23 |
| UTP14A | FLOT1 | ZCCHC9 | LIX1L | TPCN1 | NO_MATCH_119 |
| EWSR1 | CENPT | MT1B | XLOC_010217 | SLC22A7 | LPCAT4 |
| IGLL5 | MPP6 | TAGLN | NSUN7 | HEXIM2 | PLK1 |
| GPR4 | FEZ1 | HLA-DRB1 | ADCK1 | RAI1 | NO_MATCH_94 |
| CTSD | PPP1R12C | DRD2 | P2RY10 | CNDP1 | CCNG2 |
| MEF2A | LAMTOR3 | CALCA | SLC28A1 | ACTG1 | BPESC1 |
| TMEM129 | CREBRF | ZNF486 | TULP3 | ZBTB1 | TSNARE1 |
| RCHY1 | LONRF3 | COTL1 | TOR3A | SPATA2 | KSR2 |
| SMARCE1 | HHAT | GYPA | IKBKE | ATP6V1B1 | CCDC17 |
| SYBU | HEATR9 | ERVK3-1 | GLYR1 | HIGD1A | UGT1A10 |
| PIK3R1 | STX11 | HID1 | HAGHL | COG3 | EPHB3 |
| NUDT15 | LYN | SLC2A1 | IL1RAPL2 | GPR55 | PRKCD |
| BCAS1 | FAM106CP | GPR22 | PRKAR1A | TLR5 | C16orf92 |
| EPHA4 | NPY1R | NCOA4 | CDKN1A | UGP2 | NR2C1 |
| GPR132 | CPED1 | OPN1MW2 | ANGPTL2 | WDR18 | RPAIN |
| SPRTN | RFWD2 | REL | LINC00852 | PRUNE2 | KCTD19 |
| MAPT | ADARB1 | NPHP1 | PTGER3 | EEF1B2 | ATAD3B |
| COQ8A | ARHGAP20 | GOLGA7 | RSG1 | C3orf20 | PTGR2 |
| BCAN | P2RY2 | ADSSL1 | DTNA | ZNF490 | ESRRG |
| SEC22B | MECR | MAP3K8 | C3AR1 | GRPEL1 | HTR3B |
| KCNN4 | GNL3 | BTAF1 | ZBTB45 | COG4 | C5AR2 |
| MARS2 | Ddx17 | GABRR2 | NLK | USH1G | MAP3K3 |
| CPSF7 | PSEN2 | EIF3L | MKNK2 | AKAP5 | FZD9 |
| KIAA1683 | CDH13 | PITRM1 | POM121 | TNFRSF17 | FKTN |
| PANK3 | PDPK1 | PDLIM7 | GCK | CDKN2AIP | MED31 |
| GTF2A1L | EPHB6 | SNX4 | TPO | DMPK | DCAF4 |
| CTNS | RMDN3 | RBFA | CD63 | TUBGCP2 | IKBKE |
| PAK6 | CEP44 | SLC22A4 | RBM24 | BRS3 | IRAK3 |
| APP | PTGES3L-AARSD1 | IFI6 | CRY1 | TPT1 | C4BPB |
| RORA | CCDC14 | NR1H2 | TLDC1 | TAS1R3 | BMPR1A |
| PRKAA2 | MARS | FN3KRP | NEUROD2 | DNAJB4 | SCYL3 |
| MYH7 | PSMD5 | SCARB2 | GLDC | PANK3 | HMG20B |
| CDK5RAP3 | RTN4 | ACAD9 | DHX37 | ZNF134 | NO_MATCH_221 |
| ILF2 | TGFBR2 | NRBP1 | PPARG | VAMP3 | ANXA1 |
| NSA2 | SLC35A1 | SLC26A5 | SERPINB13 | REM2 | FARSB |
| CMKLR1 | PGM2 | FAM98A | AMPD3 | AK6 | CD27 |
| PPM1G | LACC1 | CHEK2 | DAXX | RHBDD1 | MT1A |
| RABGGTA | MRPL47 | LPIN3 | CCDC122 | ACVR1C | TUFM |
| CES1 | HTR2C | UGT1A1 | LMO3 | LRRTM1 | XLOC_I2_008203 |
| SOAT1 | PTPDC1 | RTFDC1 | RSPO3 | CASP5 | ASB6 |
| GALK1 | GNA15 | ATXN7L3 | TSPEAR-AS2 | LUC7L | DNAJC3 |
| EIF3J | CDR2 | XLOC_I2_004840 | TMEM33 | C1orf109 | PCDHB16 |
| TNNI3K | CXCR4 | KPNA6 | ADRA1D | TTYH3 | C14orf39 |
| DAO | DUPD1 | PRKCE | ST7 | FAM20B | SOAT1 |
| TRAF3IP2 | TRIML2 | DNAJB8 | ACSM5 | NPY5R | SPTLC1 |
| ESRRA | VASP | GC | PRPF31 | GRM4 | ALPI |
| ZCCHC7 | SLC25A25 | TSTA3 | B4GALNT2 | CDK14 | GDI1 |
| CDK19 | CAPN2 | LGR4 | PRKAG2 | C1QL2 | TMEM257 |
| NRCAM | ACKR2 | ICMT | NR2F1 | STK19 | ANXA1 |
| CHRM1 | AK5 | ADRA2C | RPP21 | FOXN2 | GALK2 |
| ABHD8 | KCNRG | JUNB | ENTPD1 | SLC26A1 | SLC33A1 |
| ENOPH1 | ERICH5 | HOMER2 | DENND6B | GSK3B | MIER2 |
| MAPK4 | TMEM121 | COMMD5 | SULF1 | CHRNA9 | LTB4R2 |
| ARHGEF3 | LPL | KMT5B | SPDEF | CDK18 | TMIE |
| CDHR3 | AMBP | ADGRE5 | SLC6A20 | UGT1A4 | SLC29A4 |
| FERD3L | RNF146 | USMG5 | Atr | FYN | ZSCAN9 |
| EMD | C1GALT1C1 | GNL2 | PDLIM3 | GK2 | FIS1 |
| KIAA0825 | LIMK1 | GRPR | KATNA1 | ZNF276 | NEDD9 |
| AGO3 | PEX16 | NO_MATCH_242 | MAPK9 | RBMX | USE1 |
| STK25 | DYNC2LI1 | ENOSF1 | MYBL1 | ARHGAP26 | CSNK2A1 |
| LCE4A | TAS1R3 | PRDX4 | CBX7 | ESR1 | NT5C3A |
| PPP1CA | SLC39A3 | PSMD3 | OR2W1 | MELK | ETNK1 |
| TMEM204 | NAT10 | ZNF563 | UGP2 | GBP2 | TKT |
| SPRY2 | LSM14A | HTR2C | PDE10A | ITK | CERKL |
| PRKAR1A | MRPS27 | GPR173 | TOMM5 | ZNF324B | SEMA4B |
| SPSB3 | CHST11 | NO_MATCH_150 | SRPK2 | ALDH3B1 | MEOX2 |
| SYVN1 | ASB3 | UST | PPP1R2 | CALR | ASIC4 |
| ARSG | XLOC_I2_009464 | RHBDL1 | ELMOD2 | SUGCT | ARPC1A |
| GFOD2 | LYPD6 | ECE2 | TPRA1 | CXXC1 | TACR1 |
| TSHR | LRRC31 | MRPS5 | GNA15 | XLOC_I2_004840 | ULK4 |
| CSNK1A1 | HIRIP3 | MFAP3 | RNASE2 | SRSF12 | ZNF101 |
| SLU7 | PCSK9 | RCOR3 | LY6E | POC1A | HNRNPK |
| IRAK1 | MAP3K13 | ACAA1 | UTP14C | FGF5 | ARPP21 |
| CCT3 | ALG12 | SHOX2 | LIPG | EVI2A | CRCP |
| ZBTB6 | TFDP3 | LINC00115 | ZNF821 | TPMT | MAPT |
| CYP51A1 | DDR2 | SLC39A1 | KIF16B | BCAP31 | GJB7 |
| KIAA1429 | HLA-DPB1 | HLA-DRB1 | KISS1R | KLHDC3 | TMED8 |
| BPHL | RGS11 | BIN2 | NCEH1 | ELOVL2 | HTR2B |
| TRAF2 | KIF1B | MTA1 | TRIM16L | VMA21 | ST6GALNAC3 |
| BMX | DPP7 | SLC29A3 | PRPH2 | ORAI1 | PPIF |
| POPDC2 | FBXL5 | ANKMY1 | INPP5K | NO_MATCH_164 | TMEM209 |
| RNF5 | JUN | VKORC1 | TAS2R38 | DUOX1 | FMO3 |
| STK24 | PTGER3 | PRKCE | IL20RA | MRPL51 | MTMR12 |
| NPR1 | RABL6 | KIAA1683 | GPR137B | PAK4 | ZNF274 |
| MCRS1 | SUGP1 | TRIM59 | TMEM14A | EYA1 | KCNG4 |
| STS | PAPSS1 | RPS2 | TF | ZNF582 | TMEM200A |
| PHIP | CCNA2 | CEP72 | NELL1 | ANOS1 | TRAK2 |
| NO_MATCH_235 | DGUOK | WNK1 | NUDT8 | GATA3 | TMPO |
| TRMT61B | ZNF512 | RORC | ALDH3A2 | RHOT1 | OPN4 |
| MCAM | KIR2DS4 | PIWIL1 | CINP | GLB1L3 | PCDHA9 |
| CNP | POLR3C | CSH1 | PHTF2 | MVK | AFF4 |
| ZMAT1 | ZNF250 | TFRC | TEX43 | GALNT2 | RIPK2 |
| ICK | PDCD2 | ZDHHC22 | CREG2 | SARAF | ACAD11 |
| YIPF5 | PDCL2 | KCNK1 | KCNJ1 | C15orf57 | DNAJA4 |
| STRC | FCN1 | MEIOC | CAMK1 | CDC25A | ING3 |
| WDR89 | DDR2 | CTAGE1 | CDKL4 | ABCF3 | GPR12 |
| DYRK2 | NDRG2 | OXTR | ASB17 | GRAMD1B | GPR107 |
| LOXL3 | PCMTD1 | HRASLS | ZNF219 | FZD7 | TBX18 |
| RAB11A | NFATC1 | POLR2F | HRH1 | NOB1 | ACTL7B |
| TNFSF13B | GLIPR1L1 | RGSL1 | POC1B | TMEM14B | CCDC28B |
| SAMM50 | C12orf50 | S1PR4 | ZNF695 | APOBEC3F | SLC34A1 |
| CDKN1A | ZNF416 | TAS2R45 | ZNF559 | GPR4 | TAL2 |
| SLC35D1 | P4HA3 | ZNF2 | CYP4V2 | LRPAP1 | IGF1R |
| GPR88 | GCKR | TSGA10 | FGFR2 | PCDHB3 | IRAK2 |
| MAGEB10 | TFB2M | TUSC2 | BCL6 | BUD13 | STK26 |
| B3GNT2 | LACTB | ID1 | DCAF15 | PRKCB | PTDSS1 |
| PGLYRP4 | SMARCE1 | SYT12 | SULT4A1 | CEACAM8 | AMT |
| HTR6 | ADAL | HTRA4 | ZBTB18 | UBE2S | RPL3 |
| HNF4A | GPBAR1 | AUH | HSP90AB1 | UBE2S | RNASE6 |
| MBNL2 | SSTR1 | GLYATL2 | DFNA5 | CLRN2 | C12orf4 |
| RPL15 | HECTD2 | CAMKV | FRS3 | TMBIM6 | SLC9A3R2 |
| MAP2K6 | BMP15 | C16orf58 | SOX2 | OR56B1 | PCDHGC3 |
| FAM13C | MTG2 | TSSK2 | CD24 | GNL2 | DNAJA3 |
| DENND1A | TBCK | KCNG1 | RPS3 | ADAMTS18 | EPN3 |
| BRCA1 | CRHR1 | PLK3 | LRP2BP | CORO2B | DFFA |
| C17orf53 | SLC4A1 | SYCP3 | MAPKAPK5 | EEF2K | PAPSS1 |
| ZDHHC7 | PTK2 | CETP | COX7B2 | NFIB | ABI1 |
| SLC25A33 | CDK4 | PCDHGC5 | VDAC2 | ARIH2 | DDOST |
| ZNF512B | BRAF | CCDC120 | POU3F2 | AFF4 | DACH1 |
| HSPB8 | UVSSA | APLNR | XLOC_000477 | AKR7A3 | RPRD1A |
| TGIF1 | EIF3E | ADORA1 | GPR162 | HBM | RPS24 |
| C16orf72 | NO_MATCH_178 | SLC13A5 | AKT3 | XLOC_I2_004129 | HIST1H2BN |
| SLC9A1 | MYRIP | DRD1 | TCN1 | TRIM28 | PLXNA4 |
| VSX2 | ACP6 | MLLT10 | PDXK | COX7C | MAK16 |
| LOC102724428 | GATSL3 | NEUROD4 | SLC2A13 | CEP83 | MRGPRX2 |
| C6orf118 | FAM71B | DNAJC11 | ZNF200 | GPC5 | CENPQ |
| ACKR3 | RFX2 | BROX | GPR161 | TAGLN2 | SLC34A2 |
| CASQ2 | SH3BP5L | H2AFZ | BRSK1 | SIGLEC6 | NR1D2 |
| PTCD1 | REPIN1 | DDB2 | NBPF19 | PFKL | PPEF1 |
| ZNF296 | DENND6A | MGLL | S100A6 | NO_MATCH_222 | ZZZ3 |
| PTPN5 | SPOCK3 | RABGEF1 | PXN | GIT2 | PDHA2 |
| TFAP2A | RPL3 | FLJ25758 | CNPPD1 | P2RY1 | PAK5 |
| GALNS | ZNF133 | LIMCH1 | RAD23B | TKTL2 | TIMM23 |
| SIGLEC10 | GNB4 | UMODL1-AS1 | GOT2 | FLCN | CLEC1A |
| NDRG1 | OR2F2 | MCCC2 | FCER1A | CLK1 | NTS |
| GALR3 | NAP1L3 | FGFR2 | DNAJC28 | CSNK1E | ADORA1 |
| OLFML2B | CD55 | SLC35B1 | FANCG | PLA2G12A | UFD1L |
| MARK2 | WDR20 | CFAP36 | HSPA8 | TRNT1 | PTGIR |
| CCDC86 | UHMK1 | TAAR1 | UNC93A | EPS8L1 | TBCC |
| PRM1 | EPHB2 | GPR3 | C1R | XPC | FDFT1 |
| SSX5 | ACVR2A | NO_MATCH_199 | C17orf102 | CAMK2D | TRPM8 |
| SRSF10 | GPLD1 | FOXRED2 | HEY1 | ZCCHC12 | CTSV |
| NEBL | NO_MATCH_113 | KRT81 | EPHX1 | STPG2 | ABHD18 |
| CDH16 | TM4SF5 | HDDC2 | TOR1A | C1orf228 | FAF2 |
| NO_MATCH_255 | METTL16 | RFPL2 | SCTR | LRRIQ1 | LMBRD1 |
| TTYH2 | SFXN1 | SNX12 | SULT6B1 | PRKD3 | HNRNPK |
| NEK7 | SBF2 | NO_MATCH_115 | UEVLD | IWS1 | PRTFDC1 |
| MYL6 | DNAJC8 | JADE1 | TRABD | ZBTB22 | LIN7A |
| KLHL34 | GNB2 | FLOT2 | ARHGAP11B | SOX10 | FAM217B |
| GPR89A | AAK1 | P2RY2 | PTPN2 | C17orf75 | ENPEP |
| DCAKD | EEF1E1 | ACER3 | GIPR | GOLT1B | OR12D3 |
| MEDAG | IRAK2 | IP6K3 | NO_MATCH_39 | CD33 | ZYX |
| HNRNPD | XLOC_I2_008259 | CRAT | ICMT | SNX1 | MTHFD2L |
| RFTN1 | PRKAA1 | CACNB4 | ZNF248 | IKBIP | UPB1 |
| MPP7 | BCCIP | MAS1L | CYP2W1 | FIBP | CEP55 |
| DDIT4 | HGF | OR5K2 | PXK | SARS | ATP5C1 |
| ZNF653 | CCDC186 | ARHGAP22 | DCTN1 | ZCRB1 | DUSP22 |
| GALR3 | TTPAL | DUS3L | ESR2 | NUDT9 | ATF6 |
| MPP2 | CPOX | GAGE12B | MID1 | PAGE1 | ITGA4 |
| APPL1 | RPS6KA4 | PBK | OR2C3 | EZR | ATXN7L1 |
| ARNTL2 | USB1 | TMEM200B | CENPN | FAM226A | SLC7A8 |
| AP3S1 | NUDC | HNRNPF | SHPK | MRPS25 | CDC37L1 |
| NELFCD | CUL1 | ABTB2 | NOCT | ERVFRD-1 | CHEK2 |
| HAS1 | RASGRP4 | PKD2L2 | TTLL2 | PPP3CC | PSMD7 |
| SLC9A3R1 | SLC18A1 | GPR108 | EPHA8 | PPIE | RAF1 |
| CXCR5 | CPLX1 | TACC1 | ADGRE1 | UBASH3B | SFRP1 |
| AKT3 | TMEM115 | SNX24 | KIF25 | ANKRD1 | PTPRE |
| CDK17 | GCK | GOT1L1 | TSPYL6 | MLH1 | SDCBP2 |
| PMEL | CYP20A1 | SLC35F1 | CBSL | PLEKHG3 | ZFP2 |
| MRPL46 | GGPS1 | P2RY12 | HSD17B12 | LATS1 | CWF19L1 |
| GRIN1 | TEX28 | TFDP2 | STK33 | DDX19B | SKA3 |
| RUFY1 | CDCA7L | CCDC155 | IRAK1 | SLC13A5 | PINX1 |
| CSK | LMNA | DHPS | KLKB1 | C10orf120 | PCID2 |
| CD53 | PPIE | POU2F1 | AKT3 | PROKR1 | CCDC127 |
| SLC43A1 | ZNF114 | PDK2 | TBC1D14 | BRD3 | TACSTD2 |
| MCCC2 | Prpf39 | CYP4X1 | TUBB1 | IZUMO2 | LOC102724428 |
| ETNPPL | RAD17 | KLHDC9 | THEMIS | PTGER3 | ZNF85 |
| FAM221B | SYAP1 | DYRK3 | LMX1A | TYRP1 | LAMP2 |
| GUK1 | HS3ST3B1 | MAPK7 | ASTE1 | GPR161 | LTV1 |
| MORN4 | Tardbp | PARVG | SIDT2 | IL23R | HOXC4 |
| TSGA13 | PMPCB | TCP11L2 | MOXD1 | LCK | NDUFA7 |
| INTS12 | GPR107 | OTUB2 | NO_MATCH_217 | SLA | RENBP |
| EIF2AK4 | TMEM199 | P4HB | PRLHR | PRKCE | DDX49 |
| GMIP | RSL24D1 | ZNF132 | S1PR5 | ZNF341 | TGFB1 |
| PRAMEF15 | TPH2 | NAGS | COMMD2 | CFAP100 | PSMB4 |
| TBR1 | MICA | DUSP10 | MAP2K7 | ACKR3 | YWHAE |
| RAP1GDS1 | CXCL11 | CLEC4M | SLC16A13 | SVOP | ITPRIPL2 |
| IL10RA | ASB7 | AGK | SLC25A1 | HPS6 | BCL7A |
| HTR1B | SERBP1 | PRR30 | AVPR1A | NROB1 | LIAS |
| C1QTNF6 | IL10 | CSGALNACT2 | CNTN5 | OSBP | PHC2 |
| ACADSB | STK38L | MED21 | TMEM139 | SLC4A8 | PPP1R14C |
| AMDHD1 | DVL1 | NO_MATCH_265 | CEP170B | GID8 | CCDC121 |
| C5orf34 | JPH3 | DHDH | NME7 | SPPL3 | GPR45 |
| PLXDC2 | ZBTB49 | CPA5 | EDIL3 | KCND1 | CCDC114 |
| RAD51D | ACER2 | TRAPPC5 | MRPS7 | MRPL32 | ANXA2 |
| ACTR3B | BEGAIN | ATMIN | PPA2 | CIRBP | RPS6KL1 |
| KLHL40 | LIX1 | SAFB2 | RCL1 | ATIC | LBHD1 |
| BIRC8 | DKK1 | NO_MATCH_20 | ADGRG5 | DNAJB14 | LGALS8 |
| CCND1 | MEX3B | PSTPIP2 | OPN1MW2 | PTMA | SLCO2A1 |
| CYB561 | BRSK2 | VDAC1 | ABLIM1 | LMBR1L | MRPL39 |
| FAM221B | PSMA4 | FGFR1OP2 | DMP1 | MGAT4B | TEKT3 |
| IL17RE | PAK5 | TRH | ADAM32 | EYA3 | CTNNA3 |
| CDK7 | LOC107984086 | MALSU1 | GTF2F1 | OR6C1 | RACGAP1 |
| PPARD | GRM6 | PRC1 | TOX4 | FN3K | ENO1 |
| CAMK2D | GUCA2B | GUSB | MAPT | PLPP5 | C10orf82 |
| GTPBP3 | AHCY | UXS1 | CD83 | LEPROTL1 | RSBN1 |
| LRRC27 | STK25 | PKIG | VAC14 | TBC1D23 | MRFAP1L1 |
| IKBKB | ZMYM5 | GPS1 | PRKCI | SMAD2 | ACOT7 |
| ZRSR2 | GPR52 | EPS8L2 | GCK | DFFB | C15orf41 |
| CSNK1G3 | DCAF4L2 | ZNF566 | XLOC_I2_007271 | ROM1 | PTH1R |
| ARFIP2 | ADPRH | TRAK1 | PAK6 | GDF5 | CLPTM1 |
| WEE1 | CECR1 | FAM151A | HGFAC | GYS1 | DCLRE1B |
| CD28 | TANGO6 | STX10 | CUTC | RNASE9 | HSF1 |
| GBA3 | TEK | CCND2 | GTPBP2 | SSC4D | REPS1 |
| PTEN | STK17B | RDH12 | KLF12 | SIRT7 | TCAP |
| HERPUD2 | DEFA6 | ASIC4 | NO_MATCH_276 | MAGT1 | SIPA1L2 |
| CYB561D2 | NLRX1 | PGAP3 | ARL15 | PRSS1 | VIPAS39 |
| TMC4 | KIF6 | EBNA1BP2 | SMYD3 | FAXC | RNF168 |
| RPL13A | PGD | RPL36AL | ELOVL1 | KLF4 | GPR135 |
| TRAP1 | MAPK13 | SH3YL1 | DNAJB9 | GRK3 | U2SURP |
| NEK2 | DDHD1 | EEPD1 | RNF217 | DCUN1D2 | ADRB2 |
| TAS2R1 | SARS2 | SLC22A23 | TKTL1 | CALCOCO2 | PRRG1 |
| SPACA1 | ZNF3 | VDR | N6AMT1 | MAP3K2 | PITX2 |
| GKAP1 | G6PC | ZBTB38 | LOC102724334 | NQO2 | WDR62 |
| ACSBG1 | YY1 | ARHGAP18 | NO_MATCH_252 | CLEC1OA | CSNK2A1 |
| NME7 | LINGO4 | C10orf88 | WWTR1 | CRAMP1 | DSTYK |
| FGF7 | WNT7A | OVOL1 | GATAD2B | RABL2B | BFSP2 |
| PBK | CLK2 | PDAP1 | TFB1M | PSMC2 | TMPRSS2 |
| CTBP1 | UBE2D3 | CCDC36 | GPR26 | CCDC105 | DEGS1 |
| BTN3A1 | HDAC1 | ALDH8A1 | RAC1 | CSNK1G1 | RPL3 |
| ECEL1 | PTAFR | HUNK | ACVR2B | AKR1C2 | OR51E2 |
| KLF2 | HHIPL2 | RASSF3 | PABPC5 | FAM3A | P2RY1 |
| FZD9 | FRMD1 | PDHX | RNF113A | RNF180 | PRR23B |
| SULT1E1 | MMP8 | TMEM134 | EOGT | GNAS | SRC |
| TWF1 | ZNF548 | LAX1 | FRK | PRKCB | EMC2 |
| ENSA | TCF3 | DACT3 | MIR650 | THRB | KIF24 |
| GPR78 | THRB | TMTC4 | AAR2 | TRIB2 | CHRM1 |
| RIN2 | SF1 | PTGER3 | FSCN2 | PLG | PPP2R2B |
| MIP | HLA-A | AGTR1 | DIDO1 | SERPINB11 | TGFBR1 |
| COL21A1 | MAP3K8 | CCR4 | NO_MATCH_143 | CHAF1B | SDCBP |
| MAN1B1 | GRB2 | SHC1 | DNAJA2 | STMN2 | SETMAR |
| S1PR1 | WASF2 | CA4 | GRAMD1C | EDDM3A | SMAP1 |
| NAPB | ARHGAP15 | ARHGEF6 | TESK1 | ARSK | XLOC_I2_004840 |
| UQCC1 | CLK2 | IFNA4 | PDHB | HLA-DQB2 | SSTR4 |
| COQ8B | No_MATCH_127 | TSSK2 | FAAP100 | DRC3 | PSG1 |
| MICAL2 | KRBoX4 | ICA1 | IRF2 | RARA | EHMT2 |
| ANXA7 | SLC22A13 | LZTFL1 | NO_MATCH_173 | TSHB | KRT6A |
| ZNF71 | OR51E2 | BCL7C | PTBP1 | UBQLNL | NECTIN2 |
| TMEM161A | GLE1 | IL7R | LOC554223 | GTF3C5 | PDGFD |
| SNAPC2 | METTL14 | NMRAL1 | PLXNB2 | SCNN1G | CBWD5 |
| NUP88 | IRAK3 | SNX16 | SYTL3 | DYRK2 | FGFR2 |
| EIF2AK2 | SPA17 | MEI1 | RIPK3 | EZH1 | ADAT1 |
| LRRC8B | STAT6 | TANK | ASNS | UPP2 | ZNF98 |
| NEK6 | NTSR1 | LZIC | C14orf37 | TRIT1 | PNOC |
| GIPC2 | EVA1A | C4orf47 | CLEC4F | TBC1D25 | TMEM79 |
| CBFA2T2 | PRODH | PGRMC2 | ZCCHC10 | OR7A17 | MAP4 |
| PPAN | PDLIM7 | FOS | LINGO1 | PKN1 | HTR6 |
| NR5A2 | HEBP2 | NPM1 | PIGF | WTAP | PPFIA4 |
| ZNF32 | TAS2R39 | WDR88 | CEP57 | SLC22A24 | SCO2 |
| DNAJA4 | FBXo16 | TGDS | HIST1H4H | TES | PGM3 |
| CCDC142 | ZNF30 | FAM153A | LIMK2 | RORB | SLC1A7 |
| HAL | FMO1 | THAP2 | PTGES3 | LRCH1 | CES3 |
| ZNF98 | NAP1L2 | CAMK4 | SLC35F1 | SLC52A1 | ENOX2 |
| TAS2R7 | TSPEAR | GBAS | SORT1 | HARBI1 | GALK2 |
| PDLIM5 | MAPT | ATP1B3 | COPS2 | FBP2 | SLC25A38 |
| DPEP3 | ESRRB | GPR176 | TMEM236 | UBL3 | PDK3 |
| GZMM | BCAN | C6orf223 | SLC25A18 | PoLR2G | FANK1 |
| XLOC_I2_006014 | ADRA2C | CCNH | SEC31B | TMCO3 | ETV1 |
| ZNF285 | GPR183 | GEM | WDYHV1 | CXCR1 | P2RY8 |
| UPK3B | PPM1D | NUP160 | HNRNPCL2 | ZNF689 | RBM4 |
| ADH5 | GAL3ST2 | FGFRL1 | TMC8 | SLC52A1 | COQ3 |
| SUV39H1 | RoS1 | ATG13 | STAMBP | PRR7 | TRMO |
| PPAN | SEC14L1 | SCAMP2 | FZD10 | RRAD | GOLGA8B |
| SAMD4A | NDUFV3 | ZBTB44 | RFC5 | GAPT | KIFAP3 |
| TXNRD1 | NLGN4Y | NO_MATCH_59 | XLOC_I2_015194 | GPD1 | SPAM1 |
| NME6 | ACKR3 | CFAP53 | SCTR | YIF1B | PPP1R15A |
| TMSB4Y | MRPL38 | GALK1 | CNOT7 | TMEM184A | ITPK1 |
| PRICKLE1 | NDUFA9 | NR2F1 | NEFM | IRAK3 | WDR27 |
| ACTL6A | ATG4A | SMYD2 | ZKSCAN1 | NO_MATCH_70 | DCPS |
| ARAF | RNF5 | HSPB8 | SCOC | QRSL1 | EIF4A3 |
| PTGIR | SH3YL1 | KCNJ13 | OLAH | GCLC | AIFM2 |
| NELFE | MTERF3 | LRMP | CGGBP1 | FAM98C | TAAR3 |
| RILPL2 | HLA-DRB3 | CCR3 | RIMKLB | TTLL9 | C8orf37 |
| GJA3 | DLGAP1-AS1 | DRD1 | CAMK1D | SRSF3 | SMCHD1 |
| CKM | HLA-DRB1 | SLC25A11 | HOXB6 | TMEM101 | KIF1BP |
| SEMG1 | ARAF | ALDH3A1 | CCDC138 | FMR1NB | NOLC1 |
| B4GALT4 | TNFAIP6 | EHD1 | MESP1 | PAX8 | LCE3C |
| RBM23 | NR5A1 | SRPK3 | EGFL6 | KBTBD7 | PHKG1 |
| NO_MATCH_32 | SSX2 | FBXL19 | BTBD18 | PROSC | FAM71D |
| GPRC5A | XLOC_005142 | NR2F6 | ARID3B | CTBP2 | XLOC_I2_015600 |
| PRKAR1B | PNMA1 | PENK | GNAZ | SFRP2 | RAB23 |
| NO_MATCH_243 | CXCL13 | PLEKHB2 | MPP1 | EGFL8 | MTERF1 |
| TMEM263 | LRTM2 | RABEPK | PDGFRL | ERVW-1 | NT5C3B |
| NAPSA | ABHD5 | RIMBP3 | CCT7 | CMSS1 | KL |
| NADK2 | SUMF2 | MAGIX | JMJD6 | ETV7 | ZKSCAN8 |
| KERA | AGFG2 | FAM117B | C14orf79 | CPEB4 | SNRPC |
| HAGHL | F2R | APOA1 | LIMK2 | CDCA7 | LRRC36 |
| IRAK3 | SPANXA1 | NO_MATCH_107 | ELMOD3 | GINS1 | STAT5B |
| CCDC96 | ADAP2 | DEPDC5 | CRY2 | CNKSR1 | PIGK |
| IDO1 | CPA1 | MYLK2 | ST3GAL3 | SHC1 | L3HYPDH |
| PIF1 | SLC25A44 | FBXL12 | XRCC4 | RPS6KA4 | LONP2 |
| GPN2 | FBXL3 | ZNF772 | EEF1G | LECT2 | MPP2 |
| SSTR3 | FAM133A | NKD2 | ESM1 | CCDC65 | EIF3E |
| SMARCB1 | CCDC6 | UPF3A | GLP1R | BRAF | C8B |
| PDS5B | EXD1 | CHN1 | FBLN5 | ATG14 | TNFRSF10D |
| CCAR2 | NR4A2 | ETNK2 | CAMKK2 | CCDC102A | RPL13 |
| GRPR | CD79A | FFAR3 | PPP1CB | TMEM206 | CCRL2 |
| OSGIN1 | EBAG9 | VBP1 | ACTRT1 | MRGPRX1 | ARMC9 |
| ENO3 | HM13 | TERF2IP | RNF220 | RNF8 | Prkd2 |
| MIR4697HG | SSTR3 | NR1H2 | HIST1H4F | SLC40A1 | IL22RA1 |
| CELA3A | SERPINB11 | EPHX4 | PNKP | HINFP | LAPTM5 |
| UBE2D3 | P2RY8 | CD1E | CDX2 | VRK1 | FOXD4L6 |
| SLC35B3 | EVA1C | CRY2 | CAPNS1 | BYSL | PSAT1 |
| AHCYL1 | C5AR1 | RPAIN | NXF5 | STRN3 | SLC39A6 |
| C16orf46 | ROR1 | ATP6V0D1 | CXorf66 | MYLK3 | PRKAA1 |
| TRIM52 | MTFR2 | MAGEB4 | MTFMT | LINC01555 | CANT1 |
| LOC100653049 | TEX101 | PDGFRB | BTN3A3 | RASL11B | NMUR2 |
| CD8A | C10orf107 | NTRK1 | DCT | SFXN5 | PSMG2 |
| SMARCD2 | ZNF414 | OR6W1P | STK25 | MMP7 | CD151 |
| MYOZ2 | PIGQ | GNAL | PRKCB | CSNK2A2 | KRR1 |
| KCNG1 | CBWD2 | CLASP1 | EIF2AK1 | LNX1 | CSNK1E |
| SWAP70 | MDH2 | DHRS4L2 | POLE3 | CCER1 | SSX3 |
| VEGFA | CDK5RAP1 | GPR87 | PILRB | COMMD3 | LSM3 |
| HLA-E | ZNF230 | NR1I2 | KRR1 | CLECL1 | HLA-B |
| DAZ2 | S1PR2 | PRKCZ | CYSLTR2 | NDUFA12 | XLOC_011321 |
| EXOSC9 | CRISPLD1 | MUC15 | AGO1 | PDIA4 | KCNG4 |
| DSCR3 | PUF60 | ZNF212 | APH1A | BTBD8 | SNAP91 |
| TXNDC11 | NFYA | TAF8 | BRD3 | VRK2 | FAM153B |
| ITGB7 | KCTD6 | BRAF | PDIA6 | PRKCG | DHDDS |
| TRIB1 | POLA2 | CLU | PLXNA4 | WDR54 | INSL5 |
| FOXJ2 | NBPF8 | SLC45A3 | SFR1 | FBXL15 | UNC50 |
| ONECUT1 | MPP4 | TBC1D31 | EIF4A2 | HMGB1 | ASH2L |
| DVL2 | KISS1R | KIAA1147 | TSSK6 | CCDC137 | FHL5 |
| PDK4 | ASPA | OSGEP | RPH3AL | NUDCD3 | ZNF816 |
| SLC45A2 | BRAF | CACNB3 | CBFA2T2 | EPHA3 | RABGAP1L |
| RBM4B | GDF10 | S1PR2 | ALOX5AP | APOA5 | NUDT18 |
| GKAP1 | LETMD1 | CYP3A7 | AP1M2 | EEF2KMT | TSGA10IP |
| TMEM192 | IDS | POTEB3 | C8orf44 | SELPLG | CDK1 |
| TRIB2 | FCHO1 | BTBD9 | TRAIP | SP8 | LGALS3 |
| DCLRE1C | PCSK9 | KCNK9 | UCK1 | TNFAIP2 | QRFPR |
| HDAC1 | CLDN7 | LRRC15 | ELF2 | HPCAL4 | FAXC |
| B4GALT1 | MS4A12 | WNK1 | LRRC28 | STRADA | KCND3 |
| SHTN1 | TAPBPL | PRELP | HIPK2 | SLC29A3 | MORF4L1 |
| TBC1D28 | KCNJ5 | PLPPR1 | TMC6 | SLC25A23 | DDR2 |
| TMEM207 | IQCF2 | PYROXD2 | LARP4 | CSRNP2 | ST6GALNAC6 |
| HLX | EIF3E | HS6ST2 | ACTA1 | ATM | RALGAPA2 |
| MS4A13 | TARP | MKNK2 | TUBG1 | FDXR | ERF |
| CPZ | BHMT2 | TXNIP | NME5 | CAV3 | BEND3 |
| UMPS | ASCC1 | GPR19 | TIGD3 | FAM3B | FRMD6 |
| CCDC96 | TRIP11 | TPRA1 | STARD3 | DAPK2 | GPR19 |
| F2RL3 | SSH2 | CETN1 | CHAT | MPV17 | TBC1D7 |
| DMRTA1 | SLC29A4 | TEX12 | UBE2A | ZNF774 | USP22 |
| SLC37A4 | MKS1 | STARD3 | CGB8 | OR1Q1 | TBCK |
| ABCC11 | SIAH1 | ACVR2B | SPINK1 | KCNQ2 | RP2 |
| CSNK1D | DBH | ELOVL6 | NO_MATCH_100 | NUP210P1 | ZBP1 |
| FAM188A | GPR174 | GOLGA4 | ALKBH1 | LMBR1 | PIGR |
| GRM8 | PPP2CB | MADCAM1 | PARVA | LAMPl | OSTF1 |
| IL13 | TRIP6 | AKT1 | NIPAL1 | CALHM2 | ZMAT2 |
| KCNK13 | TASP1 | HLA-DRB5 | VASH2 | IGIP | PRAMEF10 |
| NEK11 | OR5AK2 | HOXA3 | STK17A | LCORL | TNK1 |
| GDF3 | MRGPRG | SERPINI2 | LCMT2 | GIMAP7 | DENND2D |
| CMAS | TSHZ3 | TRIM62 | ERMP1 | PSMA1 | LYZL2 |
| WARS2 | HNRNPLL | OPN3 | RPL19 | GPR157 | ZNF354C |
| C1orf106 | SPG7 | NDRG2 | FAM127C | SPATA1 | SELENOF |
| MICU1 | ENPP6 | SPATA4 | DYRK2 | FKBP3 | PIP4K2A |
| PPARD | EIF2B4 | NFU1 | MFF | TNK1 | IFRD2 |
| HOMER3 | PPP1R12B | GCAT | RBM34 | IL12B | GABRA5 |
| GCNT2 | GCSAML | TRAF1 | CDKN3 | ARFRP1 | GOLGA6L9 |
| ACTG2 | GABARAPL2 | ZSCAN22 | HPD | SALL4 | HTR5A |
| FOXP4 | PNLIPRP2 | CNNM1 | TPPP3 | THSD4 | PRKCZ |
| PCBP3 | NAPA | PSEN1 | GRB2 | Fgd6 | TP53I11 |
| OPRK1 | NAPG | CCDC7 | NOXA1 | DMBT1 | RBM3 |
| RIOK1 | ZNF587 | P2RY8 | RSRC2 | NR1H4 | MAP2K3 |
| SSTR4 | ARHGAP36 | AGXT | TGM3 | KLK3 | DNAJC10 |
| RRN3 | CSNK1G3 | PGLYRP3 | PID1 | GDA | TMPRSS4 |
| ACSM5 | AKT3 | GPR84 | MYL6 | MS4A7 | DIRC2 |
| TRIM60 | GPR21 | GCNT3 | BTG1 | PTPN2 | PRKCA |
| FAM193B | PLAU | CPA3 | NUFIP1 | BRMS1 | TMED6 |
| ADSS | MLPH | KYAT3 | HMCES | PHACTR2 | TMPRSS11E |
| C3orf36 | RIC8A | FEZF2 | DENND1B | PGK2 | OR1E2 |
| OR2T2 | CHRM2 | GPR17 | GRK2 | TMLHE | GRM4 |
| CPNE7 | LPAR6 | HTR3B | Atp11b | SGK1 | PANX1 |
| APTX | CDK16 | KCNC4 | NPY5R | VPS4B | FOXP2 |
| KIR3DL1 | PRKCA | CLEC1B | CA10 | CHRM3 | GPR119 |
| SLC39A11 | ZNF252P-AS1 | ALDH3B2 | MELK | Srgap3 | EMC7 |
| SLC35D3 | RTCA | FNTB | NUP58 | TTC23 | NUP85 |
| Laptm4b | MATK | TMEM45B | SNAI1 | LPAR3 | GPN2 |
| LGMN | FLJ37201 | ITGB1BP2 | RPL32 | RPL35A | RPL6 |
| Rnf150 | NIM1K | CCDC12 | SPATA7 | MOGS | PYCR1 |
| CHRNA3 | RIC3 | DNAL1 | ZNF155 | COX11 | IP6K2 |
| RTP4 | TSPY10 | CXCR6 | ASB8 | OPN4 | GPC6 |
| CSPP1 | DHRS7B | ABCA8 | DPAGT1 | RAB3A | GPATCH2L |
| NFIL3 | FANCM | NFIC | MLYCD | GPR141 | FAM234A |
| TCN2 | LOC105379861 | ACAD10 | RWDD2A | GNB2 | LOC55338 |
| ALDOA | SNUPN | CXorf67 | RAET1E | CEP70 | GRM4 |
| YWHAB | Rasl10a | SYF2 | NUFIP2 | GRM3 | CCDC13 |
| CHRNA3 | EXD2 | LYZL4 | NAMPT | CHMP4C | AHCY |
| GBP7 | PGRMC1 | TBRG4 | GBP7 | IL21 | ENO2 |
| CCR1 | TMEM9 | CETP | ADCYAP1R1 | PHKG2 | SMPD3 |
| SYT2 | PCLO | DECR1 | CHSY3 | TWF1 | FAM196A |
| NDUFA5 | SSTR5 | PRLHR | GPA33 | ANKZF1 | PI4K2A |
| FEZF1 | CNIH1 | HTR3C | EDNRA | CSNK1G1 | REEP4 |
| OSBPL2 | SPRY1 | AMZ1 | WIPI2 | GCGR | SLC11A1 |
| TKTL1 | ATP5F1 | HERPUD1 | MURC | ROGDI | HOMER3 |
| GPR15 | SPATA12 | CTGF | CCDC17 | ZDHHC16 | CD209 |
| DIXDC1 | SCGB1D1 | PLAU | HSD17B2 | ZNF517 | DMPK |
| FERMT3 | XLOC_007477 | ZFP36L1 | IER2 | DYX1C1 | PMM1 |
| C3orf62 | GINS2 | CASP7 | CXorf40A | APOC3 | CYP24A1 |
| BEX5 | GCGR | HEXA | LRRC32 | UBE2Q2 | IRGM |
| PIH1D1 | STMN3 | KCNAB2 | UBTD1 | NPRL3 | ANKS1B |
| ILVBL | DTX3 | TWF2 | PPIB | IL26 | VWC2L |
| HMX2 | C9orf106 | RARS2 | ACRV1 | TRAM2 | CHUK |
| ADRA2B | TAF1D | DYNC2LI1 | MGME1 | PDGFRL | PIAS4 |
| XXYLT1 | ARL6IP4 | DVL2 | SLC5A8 | NO_MATCH_34 | EPN3 |
| ATP6V1E2 | 42987 | AKR7A3 | UXT | ZPBP | LSP1P3 |
| PSMA4 | NOL10 | DNAJC17 | IYD | HDAC3 | DYRK4 |
| MAPT | LCMT1 | P2RY6 | NO_MATCH_207 | ARHGEF19 | CYC1 |
| PACSIN2 | ESRRG | SNF8 | TMEM43 | ATP5G1 | RPS6KB2 |
| LSMEM1 | TTC7A | DCAF11 | CYP2B6 | ARRB2 | Fam122a |
| KAZN | LAG3 | RND2 | HEMGN | LRRC74A | FBF1 |
| CCRL2 | FRA10AC1 | BIN3 | VAMP5 | RORB | COQ8A |
| SFRP4 | PFKFB3 | MT1H | NR4A1 | WDR83 | NAGK |
| LINC01600 | ATF6B | TTK | GUCD1 | CLYBL | FBXO39 |
| OR10J5 | NEFL | SRSF9 | TMEM27 | NEUROD6 | PPIL4 |
| DCANP1 | RPH3A | TLE6 | PAICS | MTCH2 | P4HB |
| LDLRAP1 | MYCT1 | STBD1 | CASS4 | ATP5S | RAB6A |
| NAT9 | TRIM9 | PITPNB | BCAS1 | ATG16L1 | PHB2 |
| LILRB5 | EDNRB | DDI1 | TBC1D13 | ALDH1L1 | SSMEM1 |
| HHLA3 | PLBD1 | ZNF747 | YAF2 | KCNQ1 | HMGN1 |
| MFSD7 | RPS6KL1 | AGER | PRKAG3 | C2orf49 | GMPR2 |
| TOM1L2 | NOTCH2NL | VNN2 | TMEM50B | AFMID | NO_MATCH_47 |
| KANSL1 | MLF1 | GGTLC1 | RPL14 | KIF16B | WASF2 |
| MED11 | PRCC | NO_MATCH_257 | COX4I1 | STAP1 | CLEC3B |
| ZBTB43 | ADRB3 | KLF3 | SLC37A2 | IL10RA | PACRG |
| VPS4A | YWHAG | SLC25A6 | TUBB4A | C1orf127 | PATZ1 |
| ERLIN1 | ZNF720 | RAD23A | RARRES3 | TAF7L | HTR1D |
| SLC18A1 | GSK3A | DYRK2 | STRADA | NTF4 | CHST13 |
| ADRB1 | ZNF75D | KLHL32 | ARC | WBSCR22 | SIRPD |
| GABRD | DEGS2 | BSND | NIF3L1 | TRAPPC13 | MSC |
| TUBA4A | TRMT44 | P4HA3 | WNT16 | ESRI | NT5DC1 |
| SENP1 | SERBP1 | GJB4 | RAD51D | GSK3A | PFKP |
| SPATA1 | TAAR5 | PSMD3 | TNFSF8 | C16orf78 | YES1 |
| RCN2 | TRIM50 | C17orf51 | XLOC_002741 | MTNR1A | GHRHR |
| CCR6 | PPP2CA | EGR2 | NOTUM | 42984 | PLPP2 |
| THAP4 | RIBC2 | ABI2 | C2orf73 | RIMBP2 | PELI3 |
| NUP62 | PIGC | GBGT1 | FIBCD1 | ANKRD26P1 | OLR1 |
| DDX39B | C19orf47 | FNIP1 | SMAD4 | HAX1 | LOC100128554 |
| EEF1B2 | USP39 | PSMC1 | GPRC5C | HLA-F | GCK |
| MTNR1B | ANKRD29 | RPS14 | GPAA1 | CHIT1 | PDE1C |
| MARK2 | NPY2R | PAK6 | NATD1 | OXSR1 | SEC22A |
| TXN2 | AMN1 | C8orf34 | DNAJC15 | XPNPEP3 | SSNA1 |
| CAMK2B | NO_MATCH_224 | CXorf58 | FRMD8 | C6orf89 | CYP7B1 |
| LRRC25 | CDK18 | COG8 | LAMP3 | OTULIN | KCTD8 |
| OR1A2 | SYT17 | DGCR2 | SRSF7 | VPS53 | IFIT3 |
| NCR3 | HARS | TRAPPC10 | SNX15 | ACVRL1 | RAB24 |
| SYT9 | DTL | ALDH18A1 | FGD4 | RASGEF1A | SIGLEC7 |
| CPLX2 | HSD17B12 | MAPT | XLOC_I2_005687 | CDK20 | SEMG2 |
| PIK3R3 | LARP6 | GATAD2A | OXT | EYA4 | PTPRR |
| CDK5R1 | GABPB1 | XLOC_012729 | RAB11FIP4 | GPR18 | NSMF |
| LIN7B | CCDC102B | PHLDB3 | NDUFB6 | SLC25A42 | ROR2 |
| SLC2A14 | HLA-DPB1 | ACBD5 | TRIM2 | B9D2 | NO_MATCH_55 |
| TRA2A | PPP1R15B | DHRS13 | C15orf48 | RNF14 | P2RY1 |
| NR1I3 | HIST1H2BD | AKR1A1 | RIMKLA | MAP2K3 | XLOC_001087 |
| CD300A | MSMB | PSTPIP1 | AGO2 | BFAR | MUS81 |
| PODN | DGKA | POLG2 | NPM1 | GNA11 | FA2H |
| PSG6 | PGK1 | ADORA2A | FFAR2 | CALML4 | CRTAP |
| GMPPA | PHB | BARX1 | CCR10 | PCDHB7 | CCDC185 |
| FAM189B | C20orf96 | LACTB2 | DHODH | HCCS | STX4 |
| EPB41L4A | ZNF333 | FANCE | RIPPLY1 | ARRB2 | PNPLA2 |
| ARMC6 | SENP8 | GTF3C4 | TUBB2A | TUBB4B | XLOC_I2_014048 |
| C6orf222 | OTUB1 | PCBP1 | TYRO3 | MFSD8 | CAMK1G |
| PFKFB1 | SMOX | CXCR3 | MASP1 | KYAT3 | MAGEE2 |
| CYP4B1 | KRT18P55 | TYMSOS | PPARG | MAPK11 | NPY |
| MRPL13 | TUBA1C | DLK1 | AVIL | PSRC1 | SPIRE2 |
| ADIPOR2 | TMOD1 | LOC107984064 | CDK20 | SPATA5L1 | CAMK1 |
| BIVM | SLC23A2 | CAPG | ALDH1L2 | TAS2R8 | FZD5 |
| BHMT | GRIK2 | KCNC4 | TNS4 | ACP7 | CCR9 |
| SET | DCUN1D1 | IKZF1 | ZNF26 | AAGAB | LOC107986810 |
| KCTD4 | HNF4A | AQP1 | PGD | UBE2M | ACSL6 |
| CD59 | PHYH | FLVCR2 | BNIP3L | C9orf153 | SLX1B |
| GPR182 | ZP2 | KRT20 | LCN10 | ADSS | PRKX |
| SETD9 | PLIN5 | PPAT | KATNAL2 | USP15 | CACNG5 |
| HLA-G | GAST | ACOT1 | AK3 | OOSP2 | ASB16-AS1 |
| CSNK1G3 | AEN | PLPPR2 | MKNK1 | RNF125 | TPMT |
| RFX5 | CNGA3 | CENPK | LUC7L2 | TCTN1 | CYP46A1 |
| SEMA4B | CORO2A | KHK | SRPK2 | NFAM1 | MRM1 |
| CDC73 | ASTL | CNIH4 | COQ8B | MMP14 | FAM216A |
| METTL7A | IL20 | TACR2 | PRKAB1 | MC2R | CELF5 |
| C7orf31 | CDK2 | GNAI2 | EMILIN3 | CNPY4 | KCNMB1 |
| SLC2A12 | TEX35 | TP73 | SNRPD2 | CCDC181 | CCL28 |
| RHPN1-AS1 | TEX29 | CCT4 | CHRM5 | Vps16 | XLOC_I2_005179 |
| XLOC_013643 | CHMP5 | COCH | C16orf71 | ACADL | SPPL2B |
| CDK2 | ATF1 | P2RX1 | ZNF446 | SUGP2 | ZKSCAN8 |
| MITF | MAMDC2 | MREG | HTR5A | FGD2 | HYI |
| WDR18 | CTSD | METTL10 | RWDD2B | CAMK1D | PTGER3 |
| CHRNB3 | MLLT6 | PLEKHA1 | PIP5K1A | CYP4A22 | LTB4R |
| VWC2 | STAU2 | NXT1 | UBXN4 | ZNF843 | PBX3 |
| CLEC2B | S1PR5 | C5orf51 | SUN3 | PMPCA | NAPSA |
| ANKRD46 | SULT1B1 | LRRC41 | GPR68 | RNF128 | CHIA |
| STIM2 | SLC10A6 | TNFSF18 | RALA | PDE4D | PTGER1 |
| MKL2 | SLC13A5 | Pds5a | SLC30A5 | CCR9 | HSCB |
| SLC6A11 | PI4KB | GYG2 | PRM2 | VDAC3 | TACR1 |
| OR2T10 | FBXO21 | INTS4P1 | SMPD4 | FUT8 | GABRQ |
| CYSLTR2 | POMGNT1 | MAP3K15 | SEMA4B | THG1L | CBS |
| FAHD2A | ZBBX | LGALS13 | STK38L | CDHR4 | FMNL1 |
| SOD2 | UGT3A1 | PRRC1 | RGS9 | STK26 | HRH2 |
| SLC29A2 | CXorf38 | HIGD1A | GPR119 | CRIP1 | SCD |
| TADA3 | PINK1 | RNF126 | HIST2H4A | NO_MATCH_182 | POTEB3 |
| CD247 | CAPG | TGOLN2 | ACSBG1 | NECAB2 | TAS2R5 |
| MOCS2 | PPP2CA | LPAR5 | ABT1 | JPH4 | DAP3 |
| TSSK1B | TXNDC17 | IL17RD | CCDC90B | WASF3 | P2RY12 |
| PRKD2 | MIOX | CD300LF | UCMA | CNP | PCAT4 |
| FNDC11 | AMBN | PCYT1B | KCNK5 | DNM1 | NO_MATCH_111 |
| PYCR2 | IRF9 | PAN3 | PELO | GIPR | SSX2 |
| TDP2 | SPATS2L | SNX8 | RBBP7 | KLK13 | BAD |
| SP2 | SLAIN2 | SPATC1L | CRYL1 | HMGN1 | GPX2 |
| MYL10 | PRKAA1 | CDK15 | ANAPC15 | WEE1 | STK32B |
| SNX32 | TRIM39 | CHRNB4 | C16orf59 | GPR37 | SYT6 |
| SLC13A5 | KAT2A | CDKN1B | GADD45G | PCK2 | AGTR1 |
| HSPB7 | MON1B | C8orf86 | DRG1 | LCMT1 | SYTL1 |
| DUSP10 | SKP1 | NEK6 | TMEM30B | MSANTD2 | ACKR2 |
| PRPF18 | PBXIP1 | ZAP70 | DUSP6 | ROR2 | BMP3 |
| SLAMF6 | BTF3 | SNRPA | NAA40 | DYRK2 | HIST2H2BE |
| PPARD | RSPH9 | KCTD5 | CCDC47 | KEL | MIF4GD |
| S1PR1 | MOXD1 | SNPH | RPL37A | SCN1B | POLR2I |
| VASH1 | SNRNP35 | MFSD4A | SOHLH1 | C21orf59 | KRTAP12-1 |
| CDHR5 | FOXI1 | SMOC1 | HNRNPH2 | AMOTL2 | RIOK1 |
| HOMEZ | TMEM266 | MTX2 | LRRC4 | GNAT2 | TVP23C |
| SERPINE2 | STN1 | MPI | ISCU | VEGFC | TBC1D22B |
| H2AFY | UBE2E2 | LYPLAL1 | CXCL1 | GAS7 | RNF13 |
| CCRL2 | TRIM13 | PIN4 | NME1 | RRAGA | IGSF10 |
| METAP1 | VGLL3 | KRT38 | SPAST | ABHD13 | ZBTB42 |
| FNTA | SSC5D | SGK2 | HMGN3 | CDCP1 | IP6K1 |
| CST1 | PSMD9 | PPP2R3B | NEK2 | CDK1 | RAB3C |
| FDX1 | DMKN | BRWD1 | ARL13B | CXCR4 | CATSPER4 |
| KCNN1 | THAP11 | CELA2B | CDCA3 | SLC37A2 | DENND1B |
| SLC2A4 | CCDC174 | TRIM28 | HSD17B7 | GPR21 | TRMT112 |
| CYP2J2 | SQSTM1 | MRPS26 | GABRA3 | PHF13 | RCN1 |
| OXA1L | CD79B | GNAI3 | KRTAP9-6 | LRRC4C | TMED5 |
| LYRM1 | POP5 | NR2E1 | NDUFA10 | MARVELD2 | PDCL |
| SGO2 | KCNA7 | PTDSS1 | CCNB1IP1 | RIPK3 | NEUROD1 |
| CXorf56 | CCL19 | SLC39A5 | NKAPL | ARL4D | TRIB1 |
| THAP4 | OR2T4 | CKMT2 | HNF4G | FUNDC1 | SMAD7 |
| RNF181 | LCE1B | CLCN2 | ADORA2A | HRG | STMN1 |
| ZFYVE19 | Lztr1 | HAVCR2 | HSPBP1 | FAM76A | WBSCR22 |
| EYA2 | OR5L1 | SERPINB4 | WSB2 | VSIG8 | EFHD1 |
| EXOSC5 | CSNK1A1L | RNH1 | OPN5 | DNAJA3 | PHLDA3 |
| NR1H3 | AP2M1 | UBA7 | TMEM171 | VASH1 | LGALS8-AS1 |
| MAK | PRDM5 | CKMT1A | AKT1 | KLKB1 | SCAMP1 |
| FAM209B | P2RY14 | TFF2 | LYPD1 | ZBTB24 | PCYT2 |
| DNAJC1 | TRPT1 | HP1BP3 | SGPP2 | TRIM17 | PIGM |
| ANXA9 | RPS10 | ABHD16A | CDK6 | P2RX2 | RGS8 |
| EDN3 | EFNA5 | ADCYAP1R1 | TSPAN14 | TNFRSF10A | MINPP1 |
| KRT72 | MYL12A | PPIA | GIMAP4 | ATG9A | FOXG1 |
| KIR3DS1 | RPS20 | DOK6 | AKNAD1 | CFDP1 | MFSD2A |
| IL6R | ZAP70 | RHOJ | PHKG1 | SELENOT | NR1H4 |
| PPID | SYNPR | LOC107984065 | RMDN2 | GABPB1 | GEMIN8 |
| UTS2R | ABCA11P | ICAM1 | TSSK3 | CA9 | PDE4D |
| TYW5 | FN1 | CATSPERD | FLRT1 | CD2BP2 | RBM33 |
| PPP2R1A | ARSI | CIDEC | ADRB2 | GABPB2 | LNPK |
| CRHR1 | BAIAP2L2 | MAP2 | CAPNS1 | TSSK2 | TMEM176A |
| DPEP2 | GSTA2 | IQCF3 | PARP16 | AVPR1A | RP2 |
| BRF2 | TAAR9 | PANK2 | MSX2 | TTYH2 | MLNR |
| PHC3 | DECR2 | MSANTD3 | TPM1 | BTN3A3 | PCED1A |
| IMPDH2 | CSNK1D | INTS4P1 | SNRPG | GSK3B | TAS2R38 |
| DCTN1 | RANBP3L | PELI2 | SSX4 | RSL24D1 | GPER1 |
| CSF1R | PANK3 | CPNE1 | CDK1 | GORAB | LPAR2 |
| TNFSF9 | LZTFL1 | BCL6B | CEP41 | REEP6 | KCNC4 |
| DHFR | SPC24 | SLC39A7 | VSTM2A | NCALD | RNF175 |
| KCNMA1 | IL4 | P4HA2 | UBR2 | CAPG | SEPHS2 |
| HAUS7 | PMP2 | RAB43 | MTFR1 | ADCK5 | SLC47A2 |
| OR5M8 | LDHD | SMPD2 | CCDC34 | CYP2C9 | FOPNL |
| CXCR6 | PI16 | ZNF221 | CS | ATF4 | POLR2H |
| TESPA1 | GPR25 | XLOC_014209 | PHF7 | LINC00467 | DMKN |
| C9orf78 | P2RY13 | B4GALT2 | SLC25A32 | ITGA9 | BUB3 |
| KHK | TEN1 | MMAA | LCTL | PGRMC2 | ARHGAP29 |
| ATG10 | TMEM95 | CCKBR | LAMA4 | FABP1 | TMEM179B |
| Pkdcc | AP1AR | CAMK1G | DLX2 | TUBAL3 | RPL28 |
| ITM2C | MIF | CD99 | ZSCAN5A | DPMI | DAZ3 |
| MRPL12 | NXPE1 | KCNA3 | GALNT5 | THUMPD1 | OLFML2A |
| NFIX | CMBL | GTF3C2 | ADCYAP1 | SURF6 | XLOC_011808 |
| CSNK1A1 | MUSK | OR2F1 | KLHL7 | KRT8 | SLC39A8 |
| RAB29 | AIF1 | PRR20A | AIM1L | MTHFD2 | SCHIP1 |
| CX3CR1 | NIM1K | SPATA2L | PIP4K2C | SS18L1 | POMK |
| PCYT1A | SNX17 | DPYS | PPP2CB | S1PR3 | MRGPRD |
| GPRC5B | DHRS1 | CCT8L2 | COX7B | NGB | CRX |
| MRM3 | CIB1 | TOMM20L | AMHR2 | AZIN2 | STC2 |
| OR52I2 | NUP43 | FMO9P | MSX1 | TRHR | AK5 |
| NO_MATCH_251 | MORN3 | H2AFX | ADORA2B | FBXW11 | GGACT |
| HOMER3 | GHRHR | ODF3 | PDXK | CPA5 | DUSP26 |
| ETNK2 | TSNARE1 | GPN3 | BAG4 | PPIL1 | SNRPD2P2 |
| NPY5R | ATG13 | ATP6AP1 | GCNT1 | ATAD3B | POLD2 |
| SIVA1 | MAPK15 | CHRM4 | RELB | GATA2 | NOL7 |
| SYT5 | TRIM49 | XLOC_I2_006624 | RRP36 | DCLK3 | KIFC1 |
| PRSS23 | PIAS3 | AEN | VRK3 | OPN1SW | CCNI2 |
| EIF2AK2 | GALNT1 | HIST1H4B | XLOC_I2_001669 | CIB4 | NPBWR1 |
| PPCDC | STK17B | ARL2 | GPR142 | C7orf69 | 42979 |
| NIPBL | MGRN1 | HTR3E | PTGER4 | C1orf87 | RDM1 |
| TRPM8 | CRACR2A | TRIM69 | MAST2 | ARFGAP1 | MB21D1 |
| SLC24A5 | TREML1 | RSL24D1 | FTMT | CORO1A | FERMT1 |
| RARA | NME4 | XLOC_I2_004840 | SLC16A14 | SMR3B | GFER |
| CCDC134 | FA2H | TMOD3 | BCKDK | C1orf43 | SRPRA |
| CERS6 | ALG3 | IL2 | F2RL3 | PRR15L | DYNC2LI1 |
| NO_MATCH_180 | TCTEX1D4 | GPR37 | MAP3K12 | MRPS31 | HAPLN4 |
| PRRT2 | MAPK9 | LGALS9B | PHF23 | CALML5 | CMC2 |
| SP2 | CPA2 | HSD17B11 | PHC1 | ATP6V1G1 | CAMKK2 |
| CLDN10 | SLC6A13 | NDUFS7 | PEX13 | CCDC92 | CD1D |
| PSMA1 | LPAR2 | RBM42 | C19orf73 | DCTPP1 | CPOX |
| DUS1L | GPR156 | NONO | XLOC_I2_000791 | MRPL2 | MAK |
| TSPAN31 | FAM9B | 42795 | BCL10 | CSH2 | SLC36A2 |
| EIF3H | SLA2 | TMEM98 | MBLAC1 | SCARA5 | LOC105379861 |
| PRPF40A | TNNC1 | DCUN1D4 | FOSL2 | TKT | KCNA5 |
| PRKCZ | NAALAD2 | SERPINB2 | APOA2 | LPL | SHMT2 |
| TAB1 | PEMT | PHTF2 | LRRC3B | SYNGR2 | TMEM230 |
| KRT80 | OXGR1 | HEPACAM2 | NTSR2 | SPDYE4 | RNF115 |
| MAS1 | CDK1 | MAP1LC3B | KLK6 | INSL6 | MC1R |
| AURKA | GPR18 | RPS4Y1 | NO_MATCH_53 | LAP3 | GSTM5 |
| TOLLIP | SDC1 | NO_MATCH_93 | NSFL1C | USP30 | NR2E3 |
| FKBP6 | TRIM4 | ARMC8 | MAPK12 | DYNLRB2 | SNX18 |
| PATE1 | TTC25 | GALNT6 | NEURL3 | NKAP | EMILIN2 |
| TESK1 | ASB16 | WDFY3 | EEF2KMT | ANKRD12 | SNIP1 |
| MRPS34 | DUSP15 | OR56B4 | SEC14L3 | GPR157 | TTLL1 |
| PRAME | SHBG | FAS | C12orf43 | IL20RA | COX4I2 |
| POLDIP3 | DIABLO | DCLK3 | NAA16 | DGUOK | NUP35 |
| EPB41L4A-AS1 | XLOC_I2_009833 | HTR1B | NOL9 | C10orf111 | C16orf58 |
| PPM1K | GRAP2 | TEAD2 | BUD31 | CA1 | TPST2 |
| ZAP70 | TARBP2 | IFNLR1 | HAGHL | GNAL | NASP |
| ATAT1 | PDS5B | SPDYE1 | SCGB3A1 | HTR1F | SRMS |
| C6orf201 | DPY19L3 | SPIB | SESN3 | RAP1GDS1 | OAS1 |
| CREB5 | SLC6A2 | VKORC1L1 | WRAP73 | C1orf198 | DOK4 |
| P2RX1 | CEBPE | NUDT9P1 | F11 | TRIB3 | MAPT |
| HIST1H4E | SLC5A10 | INHBA | TMPRSS6 | FPR3 | PIP4K2C |
| DPPA3 | LINC00588 | ECI2 | PRR13 | WISP2 | CYP27C1 |
| PLA2G16 | CAPNS1 | ABCG8 | TIMMDC1 | NO_MATCH_228 | COLEC10 |
| ANXA8L1 | PSMD10 | TCEANC2 | SECISBP2 | GPR171 | CSNK2A1 |
| IMP4 | CCDC130 | LIN28B | CCR5 | FBXO2 | TGM2 |
| DOK3 | DAOA | MC1R | SPIN3 | PRPF38A | KDSR |
| WDR48 | ZC3H14 | KIFC3 | TPM4 | FLT1 | TSSK1B |
| ADH1A | DTYMK | RUSC1 | AZGP1 | THAP1 | CIAPIN1 |
| C8orf48 | HOXA6 | SERINC2 | EPHA6 | GATA3 | TMEM59 |
| HHIPL1 | ACTB | Usp32 | RNF141 | PEA15 | SSX2 |
| GPR82 | NO_MATCH_254 | DHDDS | MSL1 | GALK1 | DCAF8 |
| PLOD1 | LRRTM2 | CFL1 | PTH1R | NPR3 | C20orf141 |
| DIP2A | PRKAG3 | FCER1G | GBP4 | PAFAH1B2 | AKT2 |
| HIGD2B | THBS3 | KITLG | IL20RA | NUDT22 | TTLL6 |
| RWDD2B | WRAP53 | DAPK3 | SIGLEC12 | F2RL1 | FFAR2 |
| PNPLA1 | TTC41P | CCDC82 | BTN2A1 | MFSD5 | RSPH3 |
| PMP22 | FUT3 | AVPR2 | CIDEC | HSD17B13 | OTUB1 |
| ESAM | TOR3A | SMG8 | ROR1 | TMED7 | GBX2 |
| DYRK3 | TAAR2 | TGIF1 | ZDHHC4 | NSUN4 | CFHR2 |
| ENDOD1 | PPP1R2 | CHMP7 | CNP | DNAJC5B | UBE2J2 |
| ABLIM2 | KIAA1456 | ALG2 | XPA | ABHD4 | GNMT |
| UNC5CL | C11orf49 | ACTR1A | KRT19 | LPCAT2 | OR14C36 |
| ZAP70 | FAM212A | SDC2 | FUT10 | CDK5 | KCTD6 |
| KRTAP5-9 | SAR1A | SH3GL1 | TRAF3IP3 | AQP12B | FAM181A |
| OR4D10 | C1QBP | RAC3 | COX20 | GRB14 | POLR2E |
| RPL11 | HS1BP3 | COG4 | MRM2 | ACD | LIMK2 |
| TXLNA | ATP13A1 | CDC20 | FOXN3 | SLC39A4 | TCL1A |
| NO_MATCH_83 | TCAP | ECH1 | NXT2 | C14orf28 | OR2B11 |
| PTGIS | KCTD14 | ZCCHC11 | SNRPN | MLLT6 | CYB5A |
| ST3GAL4 | IFT22 | COPE | PLA2G2D | PLD6 | NR1H4 |
| SELENOM | DAP | FSD1L | RAB33A | ATP6V1C1 | TADA3 |
| SDHAF2 | EEFSEC | CLDN3 | LCN12 | Sgsm1 | AURKC |
| HAGH | COL2A1 | ATP6V0D2 | SLC29A1 | MMP3 | SUB1 |
| DPF1 | STRAP | SLC1A5 | CCL3L1 | NO_MATCH_248 | NO_MATCH_118 |
| LOC102724652 | PCDHGC3 | PRKCI | PIGT | JADE2 | C14orf119 |
| GPR135 | SLC35G2 | TADA1 | LGALS12 | CRYBA1 | EXOSC1 |
| BDNF | MED4 | SLAMF8 | JTB | SLC25A18 | STK25 |
| NO_MATCH_273 | ST6GALNAC5 | MAGED2 | GABRE | GIPR | FLOT2 |
| RBM18 | GNAI3 | ISY1 | RNF166 | MIA3 | CD3D |
| SRM | PIFO | CNN2 | ORMDL3 | TMOD4 | ELMO3 |
| BVES | CCDC78 | SLAIN1 | EYA3 | FBXL15 | CSNK1A1 |
| THRA | IFI27L2 | MAPK13 | RPS6KL1 | HSDL2 | DTYMK |
| TRIM62 | CDK16 | RBM42 | MORF4L1 | FOXS1 | LHCGR |
| RBKS | MAPK1 | ZBED8 | PMS1 | NR4A2 | HMCES |
| PSMB1 | PNKP | IFNW1 | HIST2H2AB | MAT1A | NXPH4 |
| DUSP10 | TYRO3 | ITPK1 | ACVR1 | GGH | CNR1 |
| FBXL17 | PIP5KL1 | CCDC146 | FAM189A1 | NOV | MUM1 |
| TCP10L | TMIGD3 | APBB1IP | ADRA1A | KHDC1 | STK38L |
| FBXO4 | DLX5 | TGIF2LY | TDO2 | DRD5 | APOL1 |
| KIRREL | RIPK3 | C19orf45 | CCDC107 | REEP2 | FNDC3B |
| FAM126B | MPP1 | WDR5B | C5orf22 | CD14 | AP1S2 |
| ATF3 | EML4 | TSC22D4 | PRDX3 | TUBB | TCERG1L |
| OTX1 | LINC01620 | MAPK3 | NUF2 | BLVRA | TACO1 |
| ADK | PSMB4 | LMAN2 | TMEM167B | GRK7 | SPHK1 |
| RPL10A | ZADH2 | TMPRSS4 | TRIP13 | S100A14 | ZNF641 |
| CCDC183 | ATP5E | ACOT8 | MEMO1 | SUOX | LIN28A |
| VN1R4 | RAB15 | SDHAF1 | HLA-C | ZFC3H1 | SAYSD1 |
| PLEKHO2 | RNF32 | IQUB | ARNTL2 | PAQR8 | CYGB |
| DFNB59 | CD82 | THTPA | ASB11 | HIST1H2AJ | LOC100130950 |
| DUSP4 | ALDOC | CDK1 | CBLN2 | SQRDL | LYPD4 |
| DNPEP | RAB11FIP1 | VWA2 | CAPN3 | RPS2 | FGF10 |
| TRIM27 | RDH11 | XLOC_I2_005151 | PCBD1 | SGSH | ECHDC1 |
| MAGOH | CHST4 | SEC61B | AIG1 | VDR | VPS36 |
| GDE1 | NR1D2 | RNF133 | TSC22D3 | MC4R | GHSR |
| Egln2 | WARS | GM2A | TPRG1L | LMO2 | ANKRD49 |
| GABPB1 | CDK5RAP1 | DBNL | LOC102724813 | C1orf43 | PARP12 |
| GPR22 | B4GALT3 | KIRREL3-AS3 | GPR139 | TMEM116 | NMNAT1 |
| OSER1 | ZC3HC1 | CCDC43 | APIP | CADMl | HTR3C |
| CHRNA7 | HAUS1 | EPHA6 | STPG3 | TWF2 | ETNK1 |
| NO_MATCH_151 | SLC25A29 | FDPS | C1orf115 | GPR12 | APLNR |
| HOXB7 | EID3 | SSFA2 | NO_MATCH_116 | PTPN18 | NO_MATCH_168 |
| STOML2 | DCAF10 | GATA4 | TBC1D21 | ADAD2 | GATAD1 |
| PNKP | KLC3 | IL6R | PIGZ | PPM1L | S100A1 |
| SORT1 | POLM | PMVK | NPRL2 | PAK1IP1 | OTOP1 |
| SAMSN1 | OPRM1 | FAM135B | PRELID2 | GPR12 | Hkdc1 |
| PPP1R1A | TACR1 | PLCXD2 | SULT1A3 | GTF2H3 | TK1 |
| CEP104 | GTF2H5 | SPATA4 | KLHL18 | HFE2 | RPL38 |
| ABAT | TRIM55 | CHST8 | MAPK8 | TMEM143 | CDK4 |
| RAN | ANKRD53 | FLJ33534 | GCSH | DSTN | ADORA2A |
| LPCAT1 | UCP1 | TMX4 | SSX1 | PRKCE | SLC17A3 |
| HMGB4 | SLC25A6 | SERPINB8 | HSF5 | GSTM1 | TTC23L |
| ZSCAN1 | MSX1 | CYTL1 | POLDIP2 | CYSLTR2 | ZNF581 |
| SH3GLB1 | SLC41A3 | ZBTB47 | CALY | MICB | GPR32 |
| TTC28 | GPR61 | SCRN2 | AUNIP | CAMK1 | DNAJC27 |
| PRKRA | BTN2A1 | NPB | ABLIM3 | SRRD | AK5 |
| NAA30 | CLEC10A | UFC1 | LOC102724151 | HEXDC | OR8D2 |
| CYB5B | MRAP | VNN1 | CTSG | GNGT1 | OR1Q1 |
| ELF2 | KLK14 | PPP2CA | ST8SIA3 | TMEM11 | TNFRSF1B |
| PIP4K2A | FN3K | SERHL2 | TSPAN11 | XCR1 | RPS6KB1 |
| NR2E1 | HM13 | AHNAK | GSDMD | CD5L | PAH |
| NO_MATCH_77 | SLC35C1 | CKM | TCTA | LRRC52 | TEF |
| ZMYM6 | CHMP2B | SEC13 | SFN | Arnt | NTN5 |
| BTG3 | NAA10 | NO_MATCH_90 | PORCN | TXNDC12 | CHI3L1 |
| AAAS | RSPH3 | DOCK8 | INHBE | BCL7A | PRSS45 |
| CDC42EP2 | ALPL | LYPD3 | AGMAT | ACSF2 | EIF2B2 |
| LOC652276 | ADAM22 | DUSP3 | TPD52L3 | RASD1 | SLC25A14 |
| XRCC3 | GET4 | CASP14 | CD38 | FFAR3 | CATSPER2P1 |
| HNMT | GOLGA2P11 | MOGAT2 | FABP6 | MTHFD2 | TRIM49C |
| NCF1 | OPRD1 | ADSL | ZFAND3 | GPR83 | MMP23B |
| SETD3 | KLRC2 | PEX3 | RCC1 | HNF4G | VNN1 |
| C19orf53 | ACTL8 | TBC1D20 | MPPE1 | XLOC_007668 | BTN2A2 |
| CEPT1 | MOB3B | TAC1 | SHMT2 | LRRC56 | EMC3 |
| NPY | RPL31 | LOC401296 | TIMD4 | AEN | SGCD |
| MYADM | HTR1F | TAT | NPY1R | GPR3 | LOC107984086 |
| F10 | SSX3 | GMFB | ARL4C | KRT18 | TMEM106B |
| GPR108 | SPANXN3 | SDHC | MRGPRX3 | SPOCK1 | LOC338797 |
| FAR2 | LYPLA1 | KCNE1 | NUBP2 | STX1B | AMIGO3 |
| OCM | BRINP1 | GALM | CAMK1D | IL26 | MRPL48 |
| ARHGEF38 | CSNK1D | TMBIM1 | ZNF707 | UBAC2 | LANCL1 |
| UBL7 | ZCCHC24 | OTX2 | RSL24D1 | IKBKG | GXYLT2 |
| ANGPTL4 | GOLGA7B | FOXR2 | NO_MATCH_188 | CA8 | LOC107984058 |
| BTN3A1 | LRIF1 | SLC22A12 | FCER2 | SOD1 | CDC25C |
| DOCK5 | FBXL19-AS1 | RPL13 | RBKS | SDHA | PI15 |
| SLC35G1 | VEPH1 | ATP5I | NEK6 | PDXK | MOK |
| SFXN3 | TWNK | C2orf50 | GPX1 | RPP38 | RAB34 |
| TNNC2 | RPUSD3 | TCTN2 | LMNTD1 | HS6ST1 | RBM4 |
| RPSA | H3F3B | ALPPL2 | C12orf74 | CSHL1 | BORCS6 |
| NXT2 | SPSB2 | HSPA4 | HBEGF | GLP1R | STX17 |
| RER1 | Sec31a | CDK5R1 | HACD4 | CSNK1A1 | PROC |
| SKP1 | TPM3 | DUOXA1 | CBX8 | OPRK1 | IL17A |
| SFT2D1 | CREB3 | ZNF343 | CSNK1E | DYRK4 | MAB21L1 |
| CCNJL | ERCC1 | CAMK2N2 | BCAT1 | NO_MATCH_49 | EIF6 |
| MPPED2 | CBR3 | OLFM4 | IFNA1 | SH2D2A | TMED2 |
| U2AF2 | P3H3 | OR1M1 | XLOC_I2_000048 | FBXL2 | NDUFA10 |
| C20orf197 | TFEB | CRNN | CPNE5 | LGALS14 | RAB14 |
| AKR1B1 | GPR162 | PCMT1 | FAM78A | STUB1 | PMEPA1 |
| ZNF428 | MRM3 | SLC6A5 | OLR1 | NUDT14 | PIP5K1A |
| MAPK10 | ZNF2 | EFNB1 | RAD9B | PGRMC2 | STYK1 |
| STAC | MAFK | SGK494 | AHSP | YIPF2 | TNFSF10 |
| ARIH1 | SMPDL3A | THOC7 | SRGN | OR2D3 | TESK2 |
| MYCBP | IFNB1 | PXYLP1 | DYNC1LI2 | PPM1K | FUT2 |
| RPS13 | STX1A | FBXO17 | RGS18 | FNDC5 | BOD1L2 |
| RPLP0 | SFTPA2 | MS4A4A | NCK1 | OR51E1 | TXN |
| TCF4 | TPD52L2 | EEF1G | SCAMP3 | C1QTNF4 | MB |
| FAM109A | GMPR2 | EMC9 | ZFAND3 | GPR63 | HIST1H2BO |
| PKMYT1 | PCGF5 | CXCR1 | RASD2 | LAMP5 | FN3KRP |
| XRCC3 | MANSC1 | ANLN | C6orf106 | ZNRD1 | XLOC_I2_009493 |
| MBTPS2 | NO_MATCH_282 | GPR3 | FFAR4 | CRYBA4 | ANTXR2 |
| TEKT4P2 | TPRA1 | LAMTOR4 | MTFR1 | RSRC1 | DRD2 |
| PTTG1 | ESR2 | DCK | GIP | TMPRSS5 | SPRYD4 |
| KCNS3 | SELENBP1 | DYRK1B | RPA2 | ELOVL3 | ASTN2 |
| IRAK1 | DAD1 | MSI2 | RPSA | APOC3 | PNKD |
| EIF2S3 | COQ8A | GSTM4 | ALDH3B1 | PGK1 | AVPR1B |
| NO_MATCH_41 | GLO1 | TPK1 | IP6K3 | KLK3 | OTULIN |
| C12orf65 | PTRH1 | GABARAPL2 | TIGIT | RPUSD4 | KIR2DL3 |
| PSTK | NO_MATCH_205 | UQCC3 | FGF1 | TACR3 | IL34 |
| SRSF2 | NO_MATCH_170 | C12orf66 | TACR3 | PI16 | MYH7 |
| UBXN1 | OR56A1 | POC1A | RGN | GPSM3 | ZNF320 |
| FECH | SPANXA1 | PTX3 | PRRG3 | SSX2 | KRTAP4-2 |
| DDAH2 | TAS2R40 | LYZL4 | KLHDC10 | ELOF1 | PIGV |
| AGFG2 | ALX3 | CCR6 | ODF4 | MYL1 | OR2L13 |
| DCAF4L1 | ZBTB26 | ECHDC2 | TRAPPC2L | NDUFS3 | CYP19A1 |
| AREG | KIF9 | DPYD | C14orf80 | POLE | FAM219B |
| PMS1 | HTR3C | HLA-DRB1 | IL1RN | PMPCB | TMEM234 |
| HDDC3 | TP73-AS1 | PAF1 | ENSA | CYP2B6 | RAP1A |
| Spire1 | LAMTOR2 | SPIC | CERS2 | CSH1 | ETNK1 |
| KDELR2 | SLC38A7 | TAAR3 | CYP46A1 | PRSS37 | MCUR1 |
| KLHL3 | HSFY2 | RBM17 | TEX33 | TUBG2 | HNRNPC |
| CARNS1 | TYRO3 | NHLRC1 | C1orf21 | CLIC2 | WDR53 |
| KHK | PRSS58 | SGK2 | AURKA | ZNF576 | HTR5A |
| ARFGAP1 | UBOX5 | PRKX | NAA20 | MED10 | CAMLG |
| TIGD6 | BMF | PYCR1 | ATP6V1D | FGFBP1 | CLIC4 |
| DLX3 | POU6F1 | SYT14 | CNR1 | CT45A3 | C3orf49 |
| CDX4 | NABP2 | OPN1MW | PLEKHS1 | OR13A1 | CDC42BPG |
| CSN2 | DCAF15 | PRL | KLK1 | MARK4 | ANKRD23 |
| RLN1 | ZNF397 | CD68 | CD4 | ARPC1B | RIDA |
| HEMK1 | ELF5 | HCAR1 | CCDC50 | MAP3K11 | ABHD10 |
| ZNF585A | GPR83 | TADA2B | LPAR2 | SAAL1 | NO_MATCH_177 |
| OR51E1 | SULT1C2 | HOXB-AS3 | DIAPH1 | PIGO | KYAT1 |
| CSN1S1 | NDUFA4L2 | ATL2 | PDIA5 | TOM1L1 | TUBGCP4 |
| KLRK1 | TICAM1 | C16orf87 | CAMK1 | CD80 | NO_MATCH_106 |
| PCED1A | PDIK1L | POP7 | MAP3K12 | CLEC17A | GHSR |
| TMEM186 | ZNF497 | SLC22A23 | INSR | C19orf57 | IQCC |
| FPR1 | ADH7 | POLR2A | PNP | AURKA | UGGT2 |
| ECSIT | MRPS24 | ZBTB32 | APIP | ZNF785 | GPR84 |
| NR0B1 | NR2E1 | SSMEM1 | PPIF | NME7 | UPK1A |
| IMPAD1 | GNPTG | USP28 | BPNT1 | POLR1D | C11orf63 |
| PKLR | IP6K3 | LOC105379861 | MEIS2 | KRT79 | LRRC46 |
| TEX26 | GPRC5A | ID3 | TMEM184C | KIAA0513 | PTGDR |
| PLD4 | SLC8A3 | IFT43 | B3GNT3 | MAPK15 | UQCRB |
| MRPL18 | ANKRD55 | NTMT1 | FUNDC2 | ENAH | COASY |
| PDLIM1 | MBP | TMEM87A | LRRC39 | EEF1AKMT1 | RPS8 |
| SYP | CAMP | SERPINF1 | CCR10 | NKG7 | NAALADL2 |
| ZMIZ2 | PRKACA | MAPK15 | ATP1B4 | MECP2 | EXOSC8 |
| RELA | PTPN18 | RBM11 | SECISBP2 | RPL14 | SLC38A5 |
| DEF8 | PRAF2 | MTHFSD | PTGER2 | CDO1 | SNRPB2 |
| CSNK1A1L | MYLK2 | PPM1M | TMCC2 | R3HDM2 | LRRN2 |
| CDX1 | AKAP9 | C2orf54 | DNAJB12 | SPCS3 | CNN3 |
| PSMB4 | TIMP4 | FTL | BTNL9 | ASPH | YAE1D1 |
| TRIM43 | ANKRD22 | NPPA | ARHGAP6 | CPSF4 | TGIF2LX |
| SLBP | SFXN1 | TEKT5 | OR51E1 | TIRAP | GP6 |
| ZNF169 | TRIB3 | CD52 | ARPC4-TTLL3 | SLC25A15 | IL20RB |
| CHIC2 | MAB21L3 | PGK1 | ACKR1 | CHMP4A | CYP2E1 |
| SLC22A7 | PIK3IP1 | SLC13A2 | STARD7 | GPR108 | USF1 |
| GAGE5 | ACOT13 | ROPN1L | ZNF254 | DAPP1 | EMP3 |
| SUCNR1 | NMUR1 | TTC1 | P2RY2 | F3 | LOC541473 |
| RNF2 | PLIN3 | SLFN5 | SPATA32 | CATSPERD | GSAP |
| XLOC_I2_009464 | TMEM185B | MVK | GPX4 | NO_MATCH_14 | GPR84 |
| DCTN4 | GIMAP5 | RPL3 | ZFC3H1 | PHPT1 | GLOD4 |
| NMUR1 | ADTRP | FAM151B | ADAT3 | TMED9 | Mok |
| MC1R | CXorf40B | FUT7 | DPM1 | ACAT2 | HTATIP2 |
| CRIP2 | RHEB | DYRK3 | FDFT1 | GATA2 | ZNRD1 |
| AMZ2 | MAD2L1BP | WT1 | ADAMTS4 | DUSP12 | B3GALT4 |
| RPLP0 | CDRT4 | IRX6 | STRADB | STK17A | KLRF1 |
| ZNF830 | TYR | FLAD1 | IKZF5 | VRK2 | GPR6 |
| TPGS2 | TSSK6 | VIPR1 | NFYB | MTMR14 | DNAL4 |
| CCR6 | PRCC | DUSP14 | LINC00526 | RAB34 | TEAD1 |
| SLX1B | FBXW4 | ZNF626 | HMOX2 | CHRFAM7A | IL20RB |
| HOMER1 | LNP1 | LINC00482 | SAP18 | GALNT10 | HNRNPA2B1 |
| AIF1L | ESD | C19orf38 | PPP1R14A | HYAL3 | HAND2 |
| GPR37L1 | FBXO27 | PNCK | PSME2 | OPN1MW | TGFBR2 |
| USP47 | MOB3C | CHIT1 | TMEM176B | HNF4A | ARHGAP32 |
| APCDD1L | NMRK2 | RXFP4 | VTN | GPR84 | PQLC2L |
| C2orf27B | CLEC12A | PBDC1 | GPR160 | MRFAP1L1 | MRPS18A |
| RABL2A | WNT10B | GCLM | RIMS3 | AQP11 | AIDA |
| BBS10 | PAQR5 | CDNF | RARB | CALM2 | ZC4H2 |
| SGCA | KRTAP10-7 | KRT73 | OR8A1 | IRGC | TIGAR |
| HVCN1 | ZFP57 | TPD52L1 | C8orf46 | HLA-DQB1 | WDR25 |
| TMEM140 | SYNE3 | NCOA7 | KLHDC7B | CHMP4A | AIM2 |
| SLC46A2 | GPR151 | CBR1 | CYB5D2 | VASH2 | EP400 |
| CLASRP | MOCS1 | PRKACG | STAU2 | CEP170P1 | ZNF169 |
| MTCH1 | AKR1C4 | NO_MATCH_153 | LMO1 | RASGEF1C | HS3ST5 |
| CYP1B1-AS1 | ATP6V1G3 | GRB7 | IL15 | BAX | RPL14 |
| S1PR5 | MTRF1L | ADORA2A | PRH2 | POLL | FSD1 |
| TRIB1 | LINC00260 | MKRN3 | BET1 | GRPR | ASF1B |
| SH3BGRL | FADD | A4GALT | NUDT21 | SPINT2 | TSSK2 |
| SEC14L3 | RAB1B | ZNF684 | TIMM17B | RAB2B | RPS17 |
| SPATA45 | KLHL14 | KLK3 | SNX12 | FUCA2 | OR1A1 |
| NO_MATCH_104 | NETO2 | OBP2A | EIF3F | HRH1 | MTFR2 |
| XLOC_I2_011954 | HBE1 | ZNF562 | AKR1A1 | ATP5L2 | ACAT2 |
| NO_MATCH_30 | GPR25 | TMCO6 | GALE | CYP1A2 | CDK10 |
| PM20D2 | TRUB1 | PCNP | AK4 | NKX2-5 | DKK2 |
| RNF25 | ADORA2B | FGF14 | SF3B6 | PTGFR | IRF3 |
| DNAJC5 | FFAR4 | C11orf42 | PGLS | SMIM12 | FAM192A |
| METTL1 | HHIPL2 | CTSL | UBQLN2 | DDX51 | NO_MATCH_270 |
| MRPS2 | AKR7A2 | STARD3NL | CCDC106 | PDE4DIP | INVS |
| NUDT4 | SELENOP | SBDS | DGCR8 | AK4 | RNF43 |
| DRD3 | NUDT1 | KCNK17 | CRHBP | SPATA24 | TFF3 |
| RAB7B | LMCD1 | RBM15B | YIPF7 | DOK2 | GPAA1 |
| MCCD1 | CD48 | NEIL1 | LINC01465 | MAPT | DENR |
| BAG2 | PSMG3 | GIN1 | MXI1 | EPYC | BCAS2 |
| PGK2 | AKR1C3 | KNSTRN | UHMK1 | TMEM177 | MRPS23 |
| HNRNPA0 | TESK2 | SLC25A5 | ODF3L1 | FANCD2OS | NO_MATCH_146 |
| CCR9 | RGR | SLC25A39 | VPS29 | MGAT5B | SPNS3 |
| MORF4L2 | GPR1 | LIN37 | SCGB1D2 | PPM1N | MVB12A |
| NO_MATCH_260 | NEU4 | NKX2-3 | ZFAND6 | POLR2L | RET |
| SPC25 | TSPAN15 | ASCL4 | SETD4 | MC5R | SCAMP3 |
| ZNF277 | MAPT | SLC5A12 | Dmc1 | THEM6 | GPR152 |
| EXOC3L2 | HLA-DRB4 | ARF1 | FPR1 | BTNL8 | KIR2DL2 |
| COX5A | HAPLN3 | PCBD2 | SPSB1 | C1orf210 | GPR182 |
| BCL7C | LARP1B | PCGF2 | PGLYRP3 | LINC00982 | CDIPT-AS1 |
| WNK1 | GPR137B | PVALB | F8 | FGFR4 | PACRG |
| FBXO6 | UROD | DLL4 | TNFSF4 | NO_MATCH_186 | RHOF |
| KRTAP9-2 | THEM5 | KDELR2 | RTKN2 | NO_MATCH_197 | DHRS12 |
| SPANXB1 | GNRHR | TXNDC8 | NTRK1 | ZNF580 | MRPL20 |
| TEKT4 | BPIFB1 | OR6K6 | STK11 | CAPS2 | NO_MATCH_24 |
| OSTC | TMEM183A | IDH3B | FAM170A | PVRIG | PBLD |
| NO_MATCH_194 | PARP11 | UTS2R | LPXN | PYHIN1 | MS4A3 |
| DPEP1 | BLOC1S5 | HTR1D | TFG | PSMB7 | FTL |
| PAX8 | ST6GAL2 | CELA3A | TTC9C | CLEC18A | DSCR4 |
| CSTA | C1orf94 | GTF2H2C_2 | C11orf52 | CDIP1 | TMEM100 |
| C6orf136 | OR13A1 | HIST1H2BJ | COMTD1 | HTR3E | CCDC103 |
| NCOR1P1 | LHX9 | MAFF | ZNF692 | GPR39 | CCDC43 |
| SPATA2L | MGST1 | PPIG | HLF | UBXN2A | CRCP |
| WDR45 | APOBEC4 | GPRC5C | XRCC2 | TRIM61 | TIMP2 |
| PRPS2 | OPRK1 | SYT11 | SURF4 | DYRK4 | PNMA6A |
| MYL12B | PTH | FAM102B | BLOC1S2 | AK4 | CDKN2C |
| MRGPRG-AS1 | TIPIN | NO_MATCH_1 | ZCCHC13 | TEAD3 | NCOA1 |
| HIF1AN | C1D | GSG1 | RAB6B | C5orf24 | SURF2 |
| RCSD1 | MMAB | CEACAM3 | FAM136A | IL17A | LINC00471 |
| ITGB3 | RAB3IL1 | TUBA1B | SPAG9 | GNGT2 | NAXD |
| PI4K2A | XLOC_I2_011911 | FAM53B | TSPAN7 | RAP2B | ARMCX1 |
| HOXD4 | NUPL2 | S100A9 | GPR171 | CCDC71 | NEGR1 |
| NIPSNAP3A | CXCL5 | IZUMO4 | EDEM2 | LINC01559 | RPRM |
| PYCARD | ZFP36 | RELA | SLC12A7 | RALB | DEFA5 |
| ZSCAN32 | GLYCTK | MYL5 | CYR61 | RNASET2 | CIR1 |
| ARSJ | SPRY2 | TRIB1 | HOXB1 | KTI12 | CABP4 |
| DESI2 | IDNK | PELI1 | LRAT | FABP7 | ZMAT5 |
| MAP3K12 | Trim72 | TMEM150A | DPY19L2 | NT5E | FAM19A5 |
| ZNF542P | BCKDHA | OR51G2 | ACMSD | AES | XLOC_I2_015213 |
| SNX21 | MRPL47 | MRGPRF | LOXL4 | TMEM18 | YBX2 |
| PAQR5 | WISP2 | AP5S1 | DALRD3 | NO_MATCH_13 | PHB |
| ARHGAP5-AS1 | TREML2 | GNB1L | WASHC3 | MUSTN1 | C1QC |
| PHF19 | CYP17A1 | UQCR11 | GPR152 | ATF5 | FAM122B |
| PEF1 | CCNJL | CHMP1A | PTMS | SLC19A3 | LRRC61 |
| UBE2B | S1PR4 | RDH13 | RAB17 | RASSF7 | NO_MATCH_169 |
| MPZL1 | CTSZ | GALK1 | TMUB2 | GALNT6 | TSSK3 |
| DMRT1 | SMPDL3B | GPR176 | MYBPHL | SCRN2 | DRICH1 |
| VMO1 | HOPX | NRAS | SLC1A7 | EGFEM1P | COPS3 |
| AKT2 | ZNF521 | Mok | MZT2A | GPER1 | MAPK10 |
| SCP2D1 | PTPN23 | SLC10A1 | TCTEX1D1 | ZNF593 | DIO3 |
| STEAP1B | HPSE | SLC35C2 | TMEM55B | BSCL2 | CRYBB3 |
| WFDC2 | PNCK | TIAL1 | EPPIN | ETS2 | NO_MATCH_206 |
| SKA2 | HIST1H3F | CHKA | GDAP1 | TGM1 | PDLIM5 |
| PANK2 | GPANK1 | GPIHBP1 | SF3B4 | HOXD10 | PAFAH1B3 |
| TARP | R3HCC1L | PLAC9 | ZBTB25 | SPP1 | C22orf31 |
| Timm8b | UBXN11 | NUS1 | INPP5K | ASB9 | MT2A |
| SELENOV | AGTRAP | TMEM41B | TTC32 | AGR3 | MAB21L2 |
| FCRL2 | NAPRT | HTR3A | ALDOB | REG4 | SCAND1 |
| CD2 | HLA-DRA | BTN2A2 | TSFM | TBPL2 | NIT2 |
| TBC1D3H | AURKC | POGZ | CRB3 | CTRB1 | APOM |
| ARPC5 | CLEC7A | ZNRF4 | PDCD5 | PKMYT1 | IL36A |
| GABARAP | FAM219A | NDP | HAPLN2 | AURKC | TAAR2 |
| DEFB129 | KCNMB4 | PFDN1 | OGFOD3 | CSH1 | HSD3B7 |
| TTC21A | ASTN2 | CSNK1A1 | KLF10 | RDH5 | RPE65 |
| RHBDD2 | TIMM10B | RNF41 | SH3BP5 | SERPINE1 | ERLIN2 |
| RAB40AL | PTP4A2 | TMEM51 | CHST10 | RCE1 | XLOC_I2_004853 |
| CSRNP1 | CYB561 | GABRR1 | IFNG | DHH | NYAP2 |
| ELK3 | GRN | TTC31 | PMF1 | HOGA1 | SNX11 |
| CAPN3 | XLOC_I2_003293 | DNALI1 | TtLL5 | SNCG | KIR2DS1 |
| PDCD6 | DNAJC12 | RNASE1 | EGFL7 | EAF1 | TAPBPL |
| DUSP21 | RPS14 | SLC22A23 | WISP1 | VSTM4 | CCDC114 |
| CCR2 | CCDC74A | C21orf62-AS1 | SLC25A37 | MLST8 | SDC3 |
| GPR68 | LGALS9C | CEACAM7 | P2RY2 | STYK1 | VGLL2 |
| ANKRD33 | KRCC1 | BAIAP2-AS1 | ABHD15 | ART3 | NEK10 |
| DPH7 | CLEC5A | CLN6 | DTYMK | EIF4EBP1 | OLR1 |
| ECE2 | SFT2D2 | PRNP | C12orf45 | ZG16 | EMG1 |
| LYRM2 | HLA-DQA1 | PSMB2 | FPR2 | GPNMB | PIP |
| PPIAL4C | FKBP11 | JTB | OLIG3 | SSH3 | ZSCAN16 |
| C2CD2 | ZNF550 | NO_MATCH_247 | NSMCE3 | ATP5L | THRSP |
| IL5RA | NO_MATCH_120 | F2RL3 | BOD1L2 | IL32 | PSMD8 |
| ICAM2 | GPR148 | RPL13A | HYAL2 | LEFTY1 | EBP |
| HSPB6 | CHRD | MCUB | SOX15 | NMUR2 | DRD3 |
| GSTO1 | SERPINA5 | DIDO1 | PKMYT1 | GPX3 | GSKIP |
| FN3K | KCNJ15 | MMS19 | OR13J1 | GPBAR1 | TMEM51-AS1 |
| GTF2I | SRPRB | ADGRG3 | TUSC3 | ROM1 | ANG |
| AP1S3 | TSPAN33 | GUCY1A3 | ADAM17 | RNF144B | GTDC1 |
| PAGR1 | BDKRB1 | NO_MATCH_157 | CRP | CLK4 | HS2ST1 |
| EIF3K | KLHDC8A | IGFL2 | RNPS1 | RABL2B | CCDC74B |
| RFFL | TIMM50 | FNDC4 | VGLL4 | FIBIN | CPLX4 |
| LPAR6 | SGK3 | C12orf10 | NO_MATCH_114 | GEMIN8 | TTC4 |
| PSG1 | RAB26 | SIN3A | CORO1C | NOSIP | MED20 |
| UBTD2 | DHRS3 | GPX1 | IHH | GJB6 | MAX |
| SERP1 | CHI3L2 | RPL15 | XLOC_I2_010511 | TIAF1 | KCNIP2 |
| TCTEX1D2 | TMEM86B | MED20 | NPTN | ZNF385B | OR2AE1 |
| COBLL1 | SEMA4G | IQCF1 | TMEM51 | BTNL3 | PCBD1 |
| SND1-IT1 | CXCR2 | SLC47A1 | ZC2HC1B | XLOC_010930 | EID2B |
| PHF19 | BGN | ALG13 | MRPS16 | TNFSF11 | ALG2 |
| SPN | TMEM89 | RGS4 | IDI1 | KDELR3 | MAPK3 |
| HTR1D | RGS5 | SLAMF9 | RET | DCAF10 | GPR18 |
| SEMA6C | PMVK | XLOC_I2_015196 | HCFC1 | ZNHIT3 | SERINC4 |
| CCM2 | FAM103A1 | GRN | STC1 | TXNDC9 | C1orf94 |
| ACKR2 | PCSK9 | MTRF1L | SURF1 | DCAF12L1 | PIPOX |
| TMIGD1 | PRDX6 | LRRC71 | NDFIP1 | DUSP4 | RTN4RL1 |
| CCL13 | TAS2R3 | NR0B2 | POGLUT1 | CDK5R1 | MT1E |
| TMEM44 | PRPS1L1 | RABIF | PDIK1L | UBL5 | IMP3 |
| PLPP6 | CCDC51 | CLEC3A | SPAG7 | TSSK3 | GH1 |
| NO_MATCH_31 | NO_MATCH_269 | GNRH1 | SPATA3 | SLC1A7 | TPD52L3 |
| LAT | NO_MATCH_267 | RASAL3 | SLC22A18 | S100A9 | DUT |
| CD300E | MMD | SPSB4 | FASLG | DCK | LINC00314 |
| OR2T4 | Scrn2 | PRPS1 | PTK6 | STAC3 | C17orf78 |
| APEX1 | DHRS1 | GRIN2B | RFK | GP6 | FCRL1 |
| GDE1 | KRTAP1-1 | ARSA | SLAMF1 | LOC107984065 | MAP4K3 |
| GCSAM | PSCA | DPY19L2 | TM4SF4 | LRRC37A | FAM177A1 |
| RNASE11 | M6PR | HYPK | ARF5 | ZCCHC16 | FAM46A |
| P2RY6 | NFKBID | XLOC_005712 | LDHAL6A | TCTE3 | NME1 |
| CCL16 | SCNM1 | BDH2 | NRN1L | NAIF1 | C15orf59 |
| VIPR1 | YOD1 | NPFFR1 | APOD | DYDC2 | ASB4 |
| MIR1-1HG | TRIB1 | 42802 | DCD | TMEM261 | TXNL4A |
| TMEM107 | RPL23 | AKT2 | LSM7 | OR6V1 | SMAD9 |
| SMIM19 | GPR162 | EFNA1 | PIANP | ZNRF2P2 | DKFZP434K028 |
| NDUFB2 | SYT3 | CLDN14 | IP6K2 | ACKR1 | EIF4EBP3 |
| KCTD21 | P2RY4 | FAM122C | VSIR | Tmem208 | TM2D3 |
| HBQ1 | GP6 | SUN1 | B3GALT5 | ATG10 | AURKA |
| RAMP3 | BORCS7 | CTAG1A | KIF26B | HRASLS5 | KRTAP19-4 |
| HSD17B6 | WDR6 | NR4A1 | FOXP1 | FXYD3 | TBX20 |
| FCGR3A | PISD | SERPINA3 | NPBWR1 | C3orf38 | VMAC |
| NFE2 | INSIG1 | HSD17B7 | URM1 | ITM2B | GAGE1 |
| CSTL1 | HEATR1 | PTGER2 | GPRC5D | GNB5 | HCN3 |
| FCGR3B | TDGF1 | RPL37 | DSCR9 | SLA2 | HN1L |
| C1orf123 | PTGER4 | SLC51A | RUFY4 | QRFPR | C8G |
| RPL28 | TWIST2 | NXNL2 | SRI | FAM63B | HIST1H1T |
| RAD9A | ZNF524 | FAM213B | ISG20 | SLC25A30 | HBB |
| XLOC_I2_006425 | TAF9B | TMEM37 | POFUT1 | HTR3A | RWDD3 |
| IL3 | SDR39U1 | COMT | STK32A | Prrc2b | PRKAG1 |
| TBP | NO_MATCH_165 | Clk3 | SNAP23 | PDLIM7 | PHTF1 |
| S100A9 | GINS2 | STAG3L4 | U2AF1L4 | CRADD | LINC00508 |
| CLDN12 | WDFY2 | SCIMP | APEX1 | B3GALT5-AS1 | P2RY14 |
| MIS12 | RNF138 | SFXN4 | UBD | SERP2 | RNF212 |
| ORAI2 | RPS16 | KLK5 | KRAS | CCDC33 | GPR157 |
| GADD45GIP1 | ZFHX2 | MYL6B | SGF29 | TAS2R13 | GEMIN6 |
| POP4 | LOC650293 | SPATS1 | NRBF2 | LHFPL4 | IL6R |
| NUPR1 | FAM8A1 | MIF | RPS3 | UQCC2 | AURKA |
| FFAR1 | PRUNE1 | NINJ1 | ELP6 | CLSTN3 | EMB |
| HYI | ODF3L2 | CRKL | EDF1 | AANAT | CCDC102B |
| SMAGP | TRHR | PRR22 | MLC1 | COA3 | SERBP1 |
| PHKG2 | SMN2 | LGALS1 | PYY | PDCD1 | MRPL43 |
| NO_MATCH_266 | DHRSX | PARP16 | AHSG | SLC25A17 | APTX |
| SNX27 | PFN3 | NO_MATCH_64 | ZNF672 | RBPMS | MBD3L1 |
| RGR | KYNU | OR10H2 | NME1-NME2 | TPM3 | MVD |
| SFR1 | TAS2R42 | ZDHHC12 | LSM12 | NAAA | TSACC |
| SAT1 | PKD1L2 | MAP1LC3A | CD300LF | MAPKAPK3 | RPS2 |
| SH2D1A | QPCT | CMKLR1 | SSBP2 | WNT9A | CLIP1 |
| AK2 | PDCD1 | C8orf74 | NO_MATCH_110 | SEM1 | GNG4 |
| TYRO3P | ARL2BP | RXFP4 | OXSM | TNFSF11 | MLLT11 |
| ZNF324 | DEFB134 | THAP8 | RAC2 | MTCH1 | EBAG9 |
| NPFF | MPG | DCN | TMEM107 | GALNT12 | XLOC_I2_009500 |
| AICDA | HN1 | PI3 | OR6B1 | CISD2 | PLPPR5 |
| RPP25L | Naa10 | SPANXD | UBA6 | GPR152 | S100A3 |
| TMEM174 | YIPF1 | IL7 | RIPPLY2 | TPRX1 | HIST1H3H |
| Rpl36a | FOXS1 | PTH2 | C1orf158 | OPN3 | HYAL1 |
| CDPF1 | FCGR1A | FOXR1 | TEX264 | RNF24 | SFTPB |
| NO_MATCH_129 | GGCT | ATP6V1G3 | UQCRQ | C7orf34 | CNPY3 |
| GH2 | DEDD2 | MTAP | CROT | ZNF718 | KLHL35 |
| IGFLR1 | FAM167B | OR6N1 | INIP | TREM1 | FAM198B |
| S100A2 | PNPLA1 | NO_MATCH_122 | PRR5 | C11orf57 | OSMR |
| SCN2B | MRPL30 | SCRG1 | PRKRIP1 | WFDC5 | DUSP3 |
| GFRA1 | OR2A2 | ADCYAP1 | ZNF544 | C16orf54 | IPMK |
| RHOQ | UFM1 | SMDT1 | RAB8B | TMUB1 | RARA |
| SIK3 | FBXO32 | TXLNGY | SLC25A22 | BLVRB | OPN5 |
| LAG3 | C1QB | MAPK3 | NO_MATCH_99 | WFDC9 | FTCD |
| XLOC_012726 | TAS2R60 | GPR142 | NRSN2 | HMP19 | SEC11C |
| C17orf64 | ENY2 | STK35 | NT5C | SERTAD3 | ZSCAN31 |
| CHRDL1 | NRP2 | NAT8L | LRRFIP1 | SOCS2 | XLOC_I2_007111 |
| TMEM55B | BIRC7 | NTRK2 | RGS8 | SECTM1 | NRG1 |
| TPO | KLF6 | AK4 | NDUFA1 | CHRNA1 | ST8SIA1 |
| PTGDR | EHBP1 | ARPC4 | REEP1 | GPR34 | IFT20 |
| MRPS18C | ANAPC10 | PNPLA5 | SPACA9 | COX5B | HIST1H4I |
| MCHR1 | TMEM35A | RAB40B | C1orf74 | ANKRD42 | ZPLD1 |
| CCL8 | PQLC1 | FAM174A | HSPB11 | XLOC_I2_010863 | MRPL57 |
| TAF12 | KRTAP13-1 | UNC119 | CH25H | PAGE2 | GINM1 |
| KCNK4 | CYB561D1 | A4GNT | DLX6 | TMEM171 | TMEM31 |
| PLPP6 | CTLA4 | OAZ1 | ANG | SUB1 | NO_MATCH_78 |
| C8orf58 | RCAN3 | PLA1A | CHCHD1 | C15orf53 | KRTAP15-1 |
| G6PC3 | XLOC_I2_009492 | GNRHR | XCL2 | CLDN4 | SMCO4 |
| DUSP7 | TTR | SMIM21 | TMEM239 | LRP1 | TUBA1B |
| CENPW | AP5B1 | LURAP1L | TEX36 | PRKAG1 | UBE2J2 |
| PDE6D | PLAC8L1 | DMRTC2 | ACOXL-AS1 | C2orf76 | NO_MATCH_171 |
| ATP5H | DERL2 | KIRREL3-AS3 | FAM189B | KRTAP13-2 | FXYD7 |
| FKBP1A | DBNDD2 | MRGPRX3 | PTPN22 | SCGB2A2 | CCDC24 |
| NPFFR1 | C2orf88 | IL9 | TIMM22 | EPO | CIB1 |
| FADS2 | ATP5D | XLOC_013551 | VSIG2 | OR2A4 | STPG3 |
| CELA2A | ERBB3 | GPR148 | DNPEP | ZFPL1 | TEX261 |
| THAP7 | BHLHE23 | LCN6 | NDUFS6 | CDCA5 | CCL5 |
| LOC100653061 | PARM1 | KLK4 | TPST2 | FOXA3 | FCGR3B |
| CELA3A | NDUFAF5 | RPS10 | HIST1H2BN | ATP6V0C | INO80C |
| DUSP13 | PERM1 | FDX1L | CNOT7 | CELA3A | RAB7A |
| LY6D | PEX11A | SEC22C | OSR2 | ATP23 | SOSTDC1 |
| EMC6 | NO_MATCH_136 | APOC2 | PAGE5 | KIF19 | VAX2 |
| HIST1H2BK | GOLT1A | IL36G | PHF5A | GNG11 | HOXB5 |
| GSTA4 | SLC10A3 | OR6M1 | RUSC1-AS1 | LOC151760 | RAMP2 |
| C1orf105 | CELA1 | CCDC126 | OBSL1 | XAGE2 | BIN3-IT1 |
| RNASE7 | GP9 | EIF4E | KLHDC9 | FAM46B | C12orf49 |
| MRPS11 | LOC400710 | MRO | SYNGR1 | ST20-AS1 | DPF1 |
| DUSP9 | ATP11B | TESMIN | TMEM14B | NKAIN4 | LINC01657 |
| FXYD3 | LINC01667 | MARCKSL1 | NDRG1 | HSPBAP1 | GPR20 |
| GPR146 | UBE2V2 | GZMB | NINJ1 | MUM1 | CLDN6 |
| ISOC2 | PTGDS | XLOC_I2_006955 | MTMR11 | HTR3D | LOC403312 |
| MAIP1 | CYSLTR1 | NFKBIB | SUMO1 | OPCML | CRYGC |
| SFTPA2 | CA13 | TACR1 | ATP5J | CXXC4 | 42796 |
| CCDC69 | NPBWR2 | LHX6 | SPATA25 | TREX1 | NXPH3 |
| RPL27A | PLA2G4D | MRAP2 | OVCA2 | FAHD2B | LINC00346 |
| KCNE5 | C6orf226 | NTM | ZDHHC16 | TTC9B | NO_MATCH_159 |
| OSTM1 | XBP1 | IL23A | FAM74A1 | NINJ2 | LOC107987001 |
| TACR2 | AXIN2 | HSBP1 | HIST1H3A | CDK3 | FXYD6 |
| PILRA | APLNR | DEFB127 | C8orf33 | APBB2 | KLK10 |
| CDC42EP1 | ABHD17A | RXFP4 | C6orf120 | CCDC189 | ACY3 |
| BATF3 | KCTD7 | COQ4 | NO_MATCH_109 | HOXA5 | SSTR2 |
| Trim41 | NTF3 | FAM180A | AGPAT2 | VAMP2 | OR2T27 |
| LDB3 | KLRC1 | FDCSP | HOXD9 | LOC146880 | PCBD1 |
| RD3 | TMEM17 | LRRC28 | TDGF1 | MAGEA1 | ANAPC13 |
| GHSR | GPRC5C | TEX264 | MORC2-AS1 | CXCL6 | RNF152 |
| NO_MATCH_44 | STX12 | NO_MATCH_167 | IFNL1 | HMGN2 | GPR180 |
| MEX3D | TBXA2R | NO_MATCH_139 | AQP5 | PLA2G15 | UBE2D1 |
| POLR2E | TM6SF1 | C11orf68 | RAMP3 | DCXR | SFTPC |
| NDUFAF4 | SLC35E1 | FRMPD4 | Ccdc28a | ROR2 | CSF3 |
| C19orf48 | SLURP1 | SPINK7 | TGIF2 | NECAP1 | GZMH |
| CKLF | FAM218A | PTGES2-AS1 | LOC105376844 | ZNF300P1 | CRYGA |
| VAMP4 | FAIM2 | LACRT | LIMD2 | CTBS | KRTCAP3 |
| NGF | GTPBP8 | YKT6 | NO_MATCH_142 | SLC10A3 | SDHAF3 |
| MT4 | OR6B2 | CER1 | PEX11B | LINC01587 | TMEM51 |
| SWSAP1 | RPS27L | TSSC4 | PTPN22 | PNPLA4 | LEFTY2 |
| EIF1B | XLOC_013281 | SCGB2A1 | AMTN | SLC31A2 | SPATA46 |
| MPDU1 | LYPD1 | FABP2 | FGFBP1 | KIF25-AS1 | PLP2 |
| PSMF1 | C9orf24 | NFATC2IP | GALR2 | IFNA2 | ATG12 |
| LOC107987205 | IFITM3 | DLK2 | KLK2 | C1orf64 | HOGA1 |
| CSDC2 | ACYP2 | C10orf55 | IFNA5 | DIRC1 | CDA |
| GNG7 | FAM124B | REG3A | KRTCAP2 | IMPA2 | TMEM136 |
| TRIM35 | CCL26 | KRTAP3-1 | GULP1 | HIST1H2AJ | LINC00479 |
| XLOC_013643 | ATOX1 | | | | |

TABLE3
RNAseq of OCI-Ly1-R vs OCI-Ly1-S

| | | | | | |
|---|---|---|---|---|---|
| SYT11 | PARM1 | MAP1A | GPM6A | ROBO2 | RGS16 |
| AOX1 | CDK6 | IGKV1-5 | RP11-539I5.1 | BEST3 | TRIM46 |
| PLCL2 | GLUL | YES1 | P4HA2 | CD48 | NOL4 |
| IGLC6 | GATM | PYHIN1 | TRO | C7orf72 | DZANK1 |
| TLR7 | CRYBG3 | RP11-284N8.3 | SGCB | PLEKHO1 | KCNA3 |
| MIAT | BACE2 | FCRL1 | KIFAP3 | DOCK10 | KIF5C |
| NPL | C14orf132 | FSD1 | SLAMF1 | NRP2 | SOX4 |
| SLC12A8 | CD69 | MORC4 | SPIRE1 | CNKSR2 | VANGL2 |
| ST20-AS1 | KIAA0040 | HMHB1 | QSOX1 | TNFRSF21 | GALNT2 |
| NAP1L3 | LY9 | CEACAM1 | RP11-544M22.13 | AMICA1 | LRP6 |
| BMPR1A | CDYL2 | CYP2U1 | CBFA2T3 | KCNMB2 | SIGLEC10 |
| PRAMENP | OPTN | ATP8A1 | STX16-NPEPL1 | BEX2 | ADAM22 |
| PDE4DIP | SNED1 | ANKRD50 | LINC00649 | ZNRF2 | NFATC4 |
| ADAM19 | TRAM2 | ARHGEF6 | CTSH | FAM198B | CD1D |
| NPNT | PAPSS1 | TWSG1 | PELI1 | IGF1R | ADARB1 |
| HEY2 | RAPGEF5 | RP11-196G18.23 | C4orf36 | CENPL | TP73 |
| ARHGAP30 | RAB39B | AFAP1-AS1 | LAMC1 | MED12L | CTD-2561B21.5 |
| MEF2D | MSANTD3-TMEFF1 | PBXIP1 | PCDHB15 | PRCC | S100A10 |
| MEGF11 | UBE2E2 | NBPF9 | CDC42EP3 | MAGEA3 | TRAF3IP3 |
| PRAF2 | GLDC | CTD-2517M22.14 | DNM3 | WHAMMP2 | LRRC8C |
| ZBTB38 | COL18A1 | DNAJC12 | RCSD1 | ATP8B2 | CHST15 |
| LINC01138 | SMCO4 | MSTO2P | SLC25A24 | CD80 | NRG3 |
| CBLN3 | PEA15 | CCDC74A | MEX3A | SESTD1 | TSTD1 |
| TRBC2 | WDR35 | NPIPB9 | LDOC1L | ASH1L-AS1 | MCL1 |
| NBPF11 | RP1-152L7.5 | TMEM2 | ABCD2 | DTD1 | RIT1 |
| RP11-876N24.1 | NUDT17 | AGMAT | SNX27 | ATRNL1 | RP11-796E2.4 |
| DUSP12 | ADPRH | NFATC2 | RPS27 | ACP6 | CHD1L |
| TRPS1 | SUCO | RNF2 | ARNTL2 | WDR17 | TMEM256-PLSCR3 |
| PHLPP1 | ASAP3 | MEST | DYRK3 | ITGA3 | SLC9A9 |
| ADAMTS6 | RP11-16K12.1 | NBPF19 | PCDHGC3 | CAND2 | CADPS |
| NETO2 | FAM163B | PYURF | NBPF15 | CDC42SE1 | ACBD6 |
| AC006978.6 | DLEU1-AS1 | TET2 | UBQLN4 | VPS72 | HACE1 |
| SLC18B1 | SF3B4 | MPP1 | ETV3 | MAGEE1 | YY1AP1 |
| RASGRP1 | IQGAP3 | MGST3 | RFX5 | RNASEL | FZD3 |
| SEC14L2 | HIST2H4B | KLHL20 | PRDM5 | UMODL1 | IFI16 |
| RP11-495P10.1 | ARHGEF2 | PIP5K1A | MRPS21 | GORAB | MPC2 |
| CICP16 | PPOX | ADIRF-AS1 | TNFRSF13B | CEACAM21 | SIAE |
| PMF1 | FCRL5 | SLAMF6 | TADA1 | ILF2 | ENSA |
| METTL18 | PROX1 | LINC00869 | CTIF | TSC22D1 | RP11-514P8.8 |
| NBPF12 | GPR89B | PDZD2 | SCYL3 | VAMP4 | CCR7 |
| SSH3 | SETDB1 | NUF2 | HAX1 | FCRL2 | QKI |
| B4GALNT1 | SGCE | AXIN2 | VWCE | RGL1 | LBH |
| CKS1B | UBE2Q1 | NBPF14 | SLC25A44 | PIGC | MYEF2 |
| VPS45 | PYGO2 | PRUNE | GPATCH4 | PITPNM2 | COLCA1 |
| S100A4 | BTNL9 | BTLA | ARNT | AC074289.1 | AZI2 |
| DTX3 | ZC4H2 | TMEFF1 | CARD16 | COPA | NME7 |
| PMF1-BGLAP | CTA-293F17.1 | HDGF | IGSF8 | SEMA4A | POGK |
| RP5-940J5.9 | APLF | MRPL9 | WI2-1896O14.1 | PELI2 | USF1 |
| XPR1 | TTC3P1 | CYP2R1 | CCT3 | LRP4 | RP11-44F21.5 |
| ZSCAN9 | RGS8 | BBS9 | RAB3IP | PSMD4 | RPRD2 |
| HEG1 | PRKCB | PFDN2 | LINC00921 | FAM102B | PTPN12 |
| RELL1 | BCL11A | PRELP | MTURN | UBASH3B | ANXA2 |
| POLR3C | AC006129.2 | AFAP1 | SLC26A1 | IRS1 | C1orf112 |
| ADAR | PI4KB | PODXL2 | C1orf220 | ANP32E | LRRN3 |
| STAMBPL1 | CREBBP | PIK3CA | PLEKHG2 | FAM167A | FMO5 |
| FIG4 | PLK2 | APBB2 | SPA17 | ARHGAP21 | PSMB4 |
| TPM3 | MARCH3 | ISG20L2 | RHOU | CH17-472G23.2 | CDHR3 |
| PHYH | DCAF8 | NCSTN | GON4L | PKP4 | MYO5A |
| HELB | ABI2 | ZNF845 | PMVK | ADA | SELL |
| TIPRL | PEX11B | GFOD1 | CGN | HIST2H2BE | PCGF5 |
| ZNF831 | SHC1 | LINC00441 | RUSC1 | TBC1D3G | APCDD1 |
| FAM175A | DEDD | UFC1 | ATP6V1G2 | USP46 | RMDN2 |
| PRDX6 | SCAPER | FOSL2 | ZBTB37 | PARP15 | PDGFD |
| RP11-404F10.2 | MSTO1 | PSD3 | CCSER2 | GGT7 | ZNF641 |
| RP11-196G18.22 | PPP1R3F | NPAS2 | RGS3 | TSEN15 | CLK2 |
| RP11-294J22.6 | GATAD2B | RASSF6 | SLC39A1 | PRPF3 | CARD11 |
| CFAP73 | RP11-458D21.5 | CH17-472G23.4 | TICAM2 | AHDC1 | RASGEF1B |
| EDN1 | SGMS1-AS1 | XXbac- | SNAPIN | SHOX2 | NFKB2 |
| | | BPG252P9.9 | | | |
| BIN1 | XXbac- | RP11-543P15.1 | INTS3 | ATOH7 | EVI5L |
| | BPG154L12.4 | | | | |
| DUSP10 | TUBA1A | CHST6 | SLK | CYP1A1 | RP11-337C18.8 |
| FER | ITPRIP | ELL2 | KCNMB4 | HDAC4 | LMO7 |
| PDE4B | CAMK4 | UBAP2L | FLAD1 | RAG1 | RASGRP3 |
| GPR155 | BCL9 | CA8 | BLZF1 | PRKAB2 | GBAP1 |
| PIAS3 | CERS2 | MAST4 | JTB | SCAF1 | ITK |
| NIT1 | PRRC2C | FCRLA | CCDC167 | CREBL2 | VPS54 |
| RNF144B | RP11-296O14.3 | DCAF6 | HIST1H1C | CCNB3 | LINC00461 |
| SSR2 | DAP3 | RAB9B | SLC19A2 | FCGR2B | PCDHGA12 |
| APH1A | NBPF20 | RP11-175B9.3 | INPP5F | SMG5 | MPZL1 |
| GCSAM | CHTOP | RNF115 | APOA1BP | SH3BP5 | BCAT1 |
| MAP2K6 | RP11-134O21.1 | UAP1 | IGLC3 | ABCB4 | PAPSS2 |
| CTTNBP2NL | CTD-2368P22.1 | DDX54 | CRTC2 | ATP1B1 | GOLGA8N |
| RBM8A | LPP | TNFSF8 | AFF1 | PEX19 | CASP1 |
| CTC-490E21.11 | XXbac- | POGZ | STARD3NL | SCNM1 | SEMA3F |
| | BPG283O16.9 | | | | |
| ZBTB7B | RP5-1028K7.2 | RP11-87G24.2 | CTD-2036P10.5 | TIAM1 | MIR3180-1 |
| RFNG | PCDH9 | RALGPS2 | ATG9B | KRTCAP2 | PRDM15 |
| ID3 | RP11-830F9.7 | AC009299.3 | C1QTNF6 | RRNAD1 | TOR1AIP2 |
| RFWD2 | CRISPLD2 | ILDR2 | TARS2 | MEX3B | SYNE1 |
| NPHP1 | ARHGAP17 | IGLV3-27 | KCNJ1 | SETD1A | GPR89A |
| ALDH9A1 | SYBU | GBP2 | MRPS14 | PEG10 | SNTB1 |
| CD84 | GAS5 | GABPB2 | IL23A | CMAHP | LRIG1 |
| LYSMD1 | PLG | RP5-1068E13.7 | SPOCK2 | NOTCH2NL | AGO4 |
| CACYBP | GARNL3 | FCRL3 | ABHD6 | AC240274.1 | MPP2 |
| METTL13 | LAMTOR2 | CTC-436K13.5 | EFNA5 | TAGAP | NBPF1 |
| ATP8B1 | RP11-452N17.1 | USP21 | GNG2 | KIAA1211 | AC091132.1 |
| C3orf52 | PCDHB14 | CEP350 | RP11-1070N10.3 | CSGALNACT2 | LINC00624 |
| MTM1 | CEP70 | SFXN5 | EFHC1 | TMEM79 | GIMAP2 |
| SNAI1 | ARPC5 | DENND4B | CP | LINC00863 | IGHG1 |
| PTPN4 | ZNF71 | STX6 | RP3-428L16.2 | RP3-455J7.4 | SPHK2 |
| TMEM14A | GPR137C | CACYBPP2 | CTSS | ARHGAP5 | RGS5 |
| UCK2 | ABL2 | MAP7D3 | TUBB2A | STK38L | KCTD17 |
| HCST | MRPL24 | RP11-132N15.3 | C11orf49 | MICA | NYNRIN |
| DPYSL2 | LRP5L | RP11-156K23.3 | OXTR | YPEL1 | MIF-AS1 |
| FBXO16 | IPO5P1 | DUSP6 | RBMS1 | ARHGEF38 | CUZD1 |
| LYPD1 | C1orf43 | IRF4 | DDAH2 | NANOS1 | ZNF653 |
| RECK | PXYLP1 | ARL3 | PFN2 | PTRHD1 | ASH1L |
| ZNF80 | TMCO1 | CH17-189H20.1 | DLGAP1-AS2 | RP11-77P6.2 | GSPT2 |
| RP11-363E7.4 | NPIPA5 | RIC8B | TCAF2 | RP11-843B15.4 | PPP2R3B |
| PPP2R3B | SRGAP2B | AIF1 | MYL12B | MLLT11 | STOX1 |
| MYO3B | LRRC1 | TSC22D1-AS1 | SRGN | UGT8 | PRRT3 |
| PLEKHA3 | SMG7 | KCNH2 | GPD2 | KYNU | ARL4C |
| NBPF3 | FMNL1 | EDEM3 | KIAA0907 | SYDE2 | CYTH4 |
| ROBO1 | FAM157A | WDPCP | CACNA1C | LRRC16A | PCNXL2 |
| SDHC | C3orf62 | AP3S1 | EGR1 | CCDC141 | TRMT1L |
| MYOM2 | RP11-564D11.3 | RP11-428K3.1 | MOB2 | LRRC70 | TOLLIP |
| PRCAT47 | SYNE3 | CCDC122 | RPGR | TMCC1 | FRMD3 |
| IGLC2 | ICA1L | CORO2B | F11R | ANKRD36BP2 | SWT1 |
| SLAIN1 | C9orf72 | CMTM8 | BDNF-AS | ATF6 | EFNA4 |
| PCNX | MAP2 | ZDBF2 | FCRLB | FDPS | PPFIBP2 |
| WDR86-AS1 | DYRK4 | SDCBP | INPP4A | CRNDE | PHF21B |
| GOLPH3L | SETBP1 | RGS5 | ZNF521 | PDCD11 | SLC36A4 |
| EFNA3 | TCEAL4 | SCAMP3 | CACFD1 | CECR1 | RP11-67L2.2 |
| HIST2H4A | FAM189B | ABCA6 | AGK | ITPR2 | HABP4 |
| ZBTB8A | LCMT1-AS1 | CTD-2162K18.4 | FILIP1L | AFAP1L2 | TMTC2 |
| ZNF493 | SEMA6A-AS1 | LLGL1 | KLHL25 | SCIMP | ZNF511 |
| SFR1 | IVNS1ABP | RP11-160O5.1 | OSER1-AS1 | SLC14A2 | AC005795.1 |
| RELB | ABHD12B | CEP126 | TPST1 | C2orf48 | ACVR2B |
| RP11-734K23.9 | GBA | AP001062.7 | PKIA | ADAMTS7P4 | PLA2G4B |
| TRIM8 | MREG | ATG4C | FAM72D | RP11-422P24.12 | WASF1 |
| NBPF26 | LRRC37A6P | POLR3GL | TRPM1 | ABCA5 | CH17-353B19.1 |
| AC245100.1 | GALNT18 | STX11 | BBS5 | RNF144A | SNURF |
| PEPD | ENPEP | TCP11L1 | ARMCX6 | ACSF3 | TNFSF4 |
| ZNF90 | DDR2 | AIM2 | ACER2 | CAPSL | FGGY |
| SOAT1 | RP11-471N19.1 | ZDHHC2 | MAP3K1 | TSPYL4 | NCF2 |
| GNA12 | IKZF5 | MAN2A1 | RP1-178F15.5 | EXOG | CNIH3 |
| PNMA1 | MFI2-AS1 | RP5-1098D14.1 | HIST1H3G | CSPG4P12 | RPS6KC1 |
| MINPP1 | RP11-326G21.1 | DARS2 | BTBD9 | PDCD4 | TGFB3 |
| SYNGAP1 | AC099342.1 | GLYATL1 | TOM1L2 | S100A6 | RIMS3 |
| PANXI | RP11-887P2.3 | CCDC17 | ZC3H12B | BLNK | CDKN2D |
| PHGDH | NCK2 | LGALSL | PYGL | ST3GAL5 | ASB8 |
| BORCS7 | RP11-876N24.2 | GADD45A | BCORP1 | RP11-290F5.1 | KIZ |
| RP11-337C18.10 | TNFAIP8L2 | NUGGC | NBPF10 | SLC50A1 | TOR3A |
| TUFT1 | MTSS1 | TAF9B | USMG5 | RPS6KA2 | NOD1 |
| ADGRB2 | BDH2 | FCGBP | RP11-437B10.1 | ANKH | HS6ST1 |
| KLF5 | ANKRD34A | ITGB8 | DNAJA4 | FANCC | RGS10 |
| DUXAP8 | CCDC28B | RP11-336K24.12 | CBX1 | ANKRD20A2 | LPP-AS2 |
| CAMK2B | STAT5B | JARID2 | SAP30 | USP27X | CALD1 |
| H3F3AP4 | IL17RA | CLGN | DRP2 | ZNF780B | TBCK |
| RP11-105N14.1 | SMIM24 | S1PR3 | FAM72C | PACS2 | USP2 |
| DUS3L | SCRN1 | MCF2L2 | ANXA11 | CCL3L3 | ATXN3 |
| ICK | ATP6V0E2-AS1 | ACTN1 | DGAT2 | TCEAL8 | JAZF1 |
| TNFRSF17 | RP11-73K9.2 | LHX4 | ACKR3 | SPRED3 | EML5 |
| SRGAP2D | AHR | RPLP2 | FBXO15 | ZNF687 | SSBP2 |
| MSRA | CFAP157 | DHX9 | SFT2D2 | SLC25A28 | SASH3 |
| RP11-53B2.6 | LL22NC03-N64E9.1 | HBEGF | NPHP3-ACAD11 | UHMK1 | RP11-159H10.3 |
| GKAP1 | FAIM | LPIN2 | SIX4 | PNOC | BTD |
| OPN3 | FZD5 | DUSP8 | RP11-126K1.6 | MAD1L1 | KDM5B |
| GLMP | LINC00278 | FAM219A | ADAM32 | PPP5D1 | TCAF2P1 |
| PIANP | KLF9 | ANKS1B | KIF20A | FZR1 | TNRC6C |
| JAG2 | ERMN | RAB25 | PJA1 | RP11-774O3.3 | RP11-522I20.3 |
| CDK5R1 | AC021106.1 | GNAZ | ZDHHC8 | SETP9 | LRRC47 |
| TLE1 | JAK1 | MTMR2 | NEDD4 | DHRSX | DHRSX |
| ENC1 | HK1 | SLC30A1 | PCGF1 | CTD-3222D19.9 | PLEK |
| ZDHHC14 | IL10RA | BCL2L2 | NT5C2 | FOXD3-AS1 | BOLA1 |
| SNHG8 | DPF1 | ARPP21 | TXNIP | ZSCAN12P1 | HIST1H2BJ |
| MGLL | B9D2 | RP11-56M3.1 | DOPEY2 | EYA2 | SMARCD3 |
| LIX1L | GHITM | CMTM7 | SSR4P1 | CBX6 | SLC10A5 |
| CNR1 | SUPT3H | RP11-352M15.2 | BTRC | PKD1P5 | IRAK1BP1 |
| BRSK1 | KATNAL2 | CD160 | SCN1B | YPEL2 | WDFY2 |
| HDAC5 | RPS13P2 | LINC01102 | RGPD6 | CORO1A | MICAL1 |
| RP11-363E6.4 | CTC-436K13.1 | TCAP | RCOR2 | STARD10 | IGKV4-1 |
| RNASEH2C | GTF2IRD1 | TNNC1 | ARMCX2 | RAB23 | AC006946.16 |
| ARHGAP44 | TAF8 | SNX24 | RAVER2 | RP11-365P13.5 | KLHL32 |
| SLC9B2 | MED12 | VARS | APBB1 | RPS6KA4 | GLTSCR1 |
| LEF1 | NPIPA8 | IFI27L2 | GSAP | RGL4 | TMSB15B |
| KCNN3 | PITPNM1 | PTEN | FSD1L | CALCOCO2 | PCDHGA10 |
| TMPRSS13 | RCN2 | ZNF583 | SBNO2 | GAK | GGH |
| AP001372.2 | POLD1 | RP11-1415C14.3 | ZNF862 | RGS13 | IMPA2 |
| PIM2 | AC093724.2 | ZC3HAV1L | RP3-382I10.7 | DCAF15 | CTA-390C10.10 |
| GSTO2 | SLC25A29 | AOC2 | NUDT4P1 | NUDT4P2 | PIGCP1 |
| IFIT5 | ZBED1 | ZBED1 | RP11-542M13.3 | CD247 | UCHL1 |
| RP1-111C20.4 | AC007040.11 | KIF16B | RP4-647J21.1 | VAV3 | NAP1L5 |
| PRR29 | RP11-278C7.3 | PIP5K1B | TERT | PAQR6 | DLGAP1-AS1 |
| C3orf18 | SEMA4C | RP11-875O11.1 | GVINP1 | RARA | RP11-620J15.3 |
| FLNA | RP11-119F19.5 | GPC1 | KIF13B | OAZ3 | THUMPD3-AS1 |
| SRP54-AS1 | VILL | PPP4R1L | TAPBPL | SH2D3C | GALNT1 |
| CDK18 | ZSCAN16-AS1 | RP11-29H23.5 | AC019118.3 | ASPHD2 | ULK1 |
| SLAIN2 | CD1C | PTBP2 | GRAMD1A | CARD10 | HCN2 |
| PPM1H | RAB37 | CSRNP2 | FAM120C | CBX4 | RP11-10K16.1 |
| ESR2 | RP11-196G18.24 | LINC00667 | CERCAM | RP11-422P24.11 | CTD-2270L9.4 |
| FNBP1L | ZNF230 | NDNL2 | MYH3 | SENP3-EIF4A1 | SYNGR3 |
| TIGIT | PLTP | AC129492.6 | RP11-93K22.13 | BRF1 | HNRNPA1P7 |
| H1FX-AS1 | AC006128.2 | ARID5B | SORT1 | CFAP20 | GAB3 |
| TESK2 | VPS9D1 | CLCN5 | BANP | POU4F1 | AL365181.2 |
| C10orf76 | RHBDF2 | USP46-AS1 | GABBR1 | TAGLN2 | RP11-121L10.2 |
| HIST1H4H | HIST1H3H | RP11-216L13.19 | RP11-706D8.3 | PDE11A | PPFIA3 |
| PLGLB1 | ST3GAL4 | NDUFS2 | STARD5 | DUSP5 | ABCB1 |
| EP300 | PRSS16 | MED25 | SMARCC2 | NATD1 | RBM12 |
| ASMT | ASMT | EVL | FZD2 | TOR1AIP1 | CCDC61 |
| RP11-91K8.4 | CAMKMT | LINC01226 | ZNF516 | TAB2 | DMWD |
| ZBED3 | RGS2 | THAP8 | PPP1R13B | RP11-641D5.1 | LPAR4 |
| RP11-398C13.6 | CBX2 | KCNMB3 | CTC-378H22.1 | PPP1R14BP3 | ENTPD1 |
| LIPT2 | TOMM40L | RP11-611O2.3 | MAP3K14 | KEAP1 | RP11-673C5.1 |
| ISL2 | ERAP2 | AKR1B1 | WAPL | TMEM265 | DGKD |
| RNF146 | SNX22 | UBQLN4P1 | C1QTNF3 | FAM110A | DLG4 |
| RP11-21K12.2 | SPRED1 | CYP4F29P | KLHL7 | ZNF571 | A2ML1 |
| METTL25 | PIP4K2A | IL16 | ZC3H4 | GPR19 | ARHGAP23 |
| TTTY15 | ZNF443 | RP11-571M6.7 | IER3 | ZMYND11 | PRC1-AS1 |
| FERP1 | GH1 | CELF2 | RUFY2 | RP11-403I13.8 | KLC2 |
| CCDC191 | FOXP1 | RP11-36C20.1 | FAM24B | CHD3 | TEX41 |
| SH2D2A | MAP1S | RP11-706O15.5 | GOLIM4 | MXRA8 | DPM3 |
| TSPAN18 | ACBD7 | CBX7 | HIST1H2AC | TMEM254 | TMEM170B |
| EHMT1 | ZNF850 | RP11-35G9.5 | BZRAP1-AS1 | DEPDC1B | GUSBP9 |
| RP11-404F10.6 | C4orf32 | CDC42BPB | GABARAPL1 | RP11-481J2.2 | SPATA7 |
| TOX2 | EFNB2 | MAGED1 | USP11 | POU2F2 | ATP6V1B1-AS1 |
| KLRC2 | AICDA | ZNF438 | HIVEP2 | MMD | RP11-197N18.7 |
| ACRC | RP6-74O6.2 | CYB5R2 | LENG9 | LINC01559 | MTCL1 |
| GLUD1 | C1orf198 | PGBD1 | PLPP1 | GLTSCR2 | ZEB2 |
| NUDT6 | B4GALT3 | CTD-2336O2.1 | CH17-373J23.1 | RP11-771K4.3 | FCGRT |
| SCN4A | HIST1H2BD | PSRC1 | CTD-3203P2.1 | IKZF3 | FAM153B |
| AC004623.3 | ARL4D | RP11-277B15.3 | ALDH16A1 | CALHM2 | TIGD3 |
| AC116366.5 | HIST1H2BL | FAM195B | MARCH9 | PHC1P1 | FNDC5 |
| RP11-464F9.1 | TPM4 | RBM11 | HIST1H2BC | ITGB2 | LINC01481 |
| PHC1 | MAPKBP1 | AC010642.1 | ST6GALNAC6 | JADE3 | HORMAD1 |
| USP12 | TGFBR2 | C22orf34 | TYK2 | IGF2BP2 | ANO6 |
| DKFZP434I0714 | RP3-486I3.7 | HSF2 | GALC | CTNNA1 | MYO1G |
| FAM156B | TTC24 | FAM89A | HIST2H2BF | KIAA0355 | SSX2IP |
| LTBP1 | FOXD2 | CTD-2270P14.5 | PAK1 | GPATCH2L | RP11-1280N14.3 |
| HECTD2 | XPNPEP1 | PIK3R3 | RP11-76I23.7 | ZNF385A | DNAH11 |
| RP11-328P23.4 | KIAA1614 | KCNC3 | XXbac- | ATRN | NCEH1 |
| | | | BPG299F13.17 | | |
| DHFRP1 | ZNF765 | ARL10 | FAM20B | RP11-367G6.3 | RFPL4AP1 |
| TMEM67 | SIT1 | CTD-2521M24.6 | FAM45A | KRT18P34 | RABGAP1L |
| IDS | MYLK3 | RP11-1079K10.3 | TAF5 | FAM212B | ANG |
| PDK3 | CA5B | RP11-1070N10.6 | ZNF559 | LIPE | C11orf24 |
| TP53BP2 | H1FX | AKIRIN2 | TOP1MT | AB019441.29 | KBTBD11 |
| AC093642.5 | SRRM2-AS1 | AKT3 | CAPN10-AS1 | FAM215B | GPR161 |
| RNF19A | RAD51B | MANF | PKD1 | ABAT | OVOS2 |
| RAB11FIP2 | PKD1L1 | ZBTB42 | CTC-490E21.14 | ACTN4 | KCNIP4 |
| CCDC112 | RP11-133K1.7 | SBF2 | RP11-20I23.13 | RP11-10N23.2 | RP11-553L6.2 |
| RP11-111F5.8 | BAG3 | FAM174B | LIMK1 | RPL23AP21 | SORL1 |
| TNRC6B | EHD1 | RP11-967K21.1 | KIF11 | OGG1 | HPSE |
| HLA-DQA1 | RP11-53B2.3 | KCNA2 | EGR2 | RP11-500C11.3 | SNRPEP4 |
| C16orf74 | KLHL6 | RAP2C | GPR55 | CGRRF1 | C11orf84 |
| GS1-358P8.4 | PDE6D | DOCK4 | RP11-175K6.2 | TMEM216 | ZNF439 |
| MAP3K4 | TCF7 | RP11-67C2.2 | ZSWIM5 | FAM168A | R3HCC1L |
| CCT8P1 | TCF3 | ACACB | STK39 | EGLN1 | LDB1 |
| CA14 | DIRC2 | L3MBTL3 | LLNLR-245B6.1 | WDR27 | FKBP7 |
| RASGRF1 | EXOSC1 | PAFAH1B3 | RP11-312J18.5 | MIR34AHG | XXYLT1 |
| ANKRD13B | RAP1GDS1 | CTD-3222D19.12 | PRKCA | KCNK12 | SLC1A2 |
| ZBTB39 | ELL2P1 | TUBGCP2 | UBE2C | RP4-570O12.3 | EHBP1L1 |
| NELL2 | FAM161A | HEBP2 | KCTD12 | LINC01297 | RP11-175K6.1 |
| RP1-178F15.4 | CEP170P1 | S100A1 | IRF6 | RAB11B-AS1 | FTO |
| B3GLCT | CTD-2287O16.5 | RP11-343C2.12 | ZNF35 | VPS41 | IGLC7 |
| HGS | CUX1 | GOLGA2P7 | RP11-797H7.1 | PTPRU | OGFR |
| CBX8 | AC241585.2 | RAD51 | ALDH18A1 | SAMD4A | ZNF708 |
| FST | RP5-940J5.6 | NUTM2A-AS1 | AKAP3 | PDCD2L | EPOR |
| KIAA0319L | AC072062.1 | DDX10P2 | ADGRL1 | ARHGAP19 | ZNF816 |
| APITD1-CORT | RP1-193H18.2 | ANKRD6 | TRIB1 | MARCKSL1 | MX2 |
| LOXL2 | RP11-517A5.5 | ANTXRLP1 | CDC14A | PFN1P8 | RAB3B |
| PIPSL | EGLN2 | RP11-467H10.1 | RP11-468E2.5 | RNF25 | IQCD |
| MYB | SIX5 | CTC-453G23.8 | MT2A | AKAP5 | AP003068.23 |
| RP11-110I1.14 | ETV6 | MSANTD3 | CD2BP2 | MURC | LGALS1 |
| RAG2 | ANKRD24 | RAP2A | ATL1 | RP11-696N14.1 | MTX1 |
| SLC35D2 | ZNF93 | SYT17 | C4orf3 | OVOL2 | NR5A2 |
| ZCCHC18 | BIRC3 | ZFP30 | RP11-761N21.2 | WBP5 | EVC2 |
| RALGPS1 | UMPS | MRO | CTBP1 | YPEL5 | KMT2B |
| FAM221A | CTD-2562G15.3 | RNF24 | APOC3 | PPP2R2D | KATNB1 |
| OTUD7B | RP11-286N22.6 | OVAAL | IGLV10-67 | RP11-753C18.12 | GNA11 |
| RN7SK | SPINK2 | NAGK | RP11-34F20.7 | RP11-573D15.9 | GLRX |
| ARHGAP25 | TIAM2 | CEP55 | TRIM17 | BCL6 | AC144449.1 |
| AP001432.14 | MZF1-AS1 | METTL12 | IKZF1 | FABP5P7 | ZNF177 |
| CD244 | KCTD1 | CTB-31O20.4 | SAMD9L | RP1-267L14.6 | NLK |
| LYRM4 | APOC4-APOC2 | TRIM3 | HMGN3 | MARCH2 | CAMSAP1 |
| SPATA5 | THEM4 | CTNNB1 | RP3-467N11.1 | MYL12A | ASB2 |
| PCDHGA1 | PPP1R26 | RP11-506M13.3 | CABLES2 | MAP3K14-AS1 | CAPS |
| U47924.6 | RAB31 | FAM57A | KIAA1024 | BCL2L1 | TTC31 |
| RP11-757F18.5 | FGD2 | RP11-196G11.1 | PHC3 | UXS1 | KMO |
| CTD-2545G14.6 | EAF1-AS1 | CIB2 | GTPBP1 | NUDT4 | ARID1B |
| FTX | CCNYL1 | CASP9 | ALG14 | CH17-174L20.1 | SNORA59B |
| MAPRE2 | RP11-713N11.5 | CASK | MARS | TSPAN11 | RAB11B |
| C2CD3 | CLDN4 | RP11-466A19.8 | RUSC1-AS1 | ZNF136 | KCNK9 |
| CCDC186 | BBIP1 | B4GALNT3 | MSI2 | RRN3P3 | FAM222B |
| SPOCD1 | RP11-308D16.2 | PSIP1 | ZFYVE27 | SGK1 | ERCC1 |
| TUBB3 | LINC00854 | KCNJ16 | ST3GAL3 | CREB3L4 | TAPT1 |
| CTD-2583A14.10 | LINC01311 | RP11-87G24.6 | FZD8 | TLDC1 | NOLC1 |
| ZYX | RP11-328C8.4 | ALS2CR12 | ST13P3 | C18orf32 | AVPR2 |
| DNMBP | TBC1D23 | KHNYN | PTPN9 | RGS20 | RP5-894A10.6 |
| NUDT22 | RP11-131L12.3 | EIF1B-AS1 | C2orf42 | RNF41 | SETP14 |
| TFDP2 | CD5 | CTB-50L17.8 | NOMO3 | C1orf226 | SPATA13 |
| C17orf53 | EXTL3 | RAB11FIP3 | CTD-2319I12.10 | SLC39A10 | HIST2H2BD |
| FLOT1 | RP11-566K11.4 | SON | CAPN10 | GPLD1 | ATP2C1 |
| LINC01054 | RP1-223E5.4 | BAIAP2L1 | RP11-624M8.1 | CNPY4 | RRP12 |
| RP11-466F5.8 | TMEM129 | LEPR | MICE | MTA3 | TM9SF3 |
| CXorf21 | FUT8 | RP1-153G14.4 | U1 | ZFP82 | CEND1 |
| ZXDB | SLA | AF011889.5 | PGM2L1 | PLEKHM1 | TPM1 |
| RP11-378A12.1 | BMI1 | RABL6 | RPLP0P6 | AGO3 | C6orf1 |
| RP11-26J3.4 | NRL | Z84812.4 | DDA1 | LY86-AS1 | TTC3 |
| C14orf79 | RP11-1319K7.1 | MIF | AC006129.3 | AC009303.2 | KIF15 |
| CYB561D1 | UBE2FP1 | CRHR1-IT1 | KDM1B | KCNAB2 | SPIN1 |
| HOXC4 | FMOD | SSBP3 | RP11-532F6.3 | AGAP7P | GBF1 |
| KIAA0430 | DDIT3 | HIST1H2AI | MPZ | C5orf66 | TUBB6 |
| TPH1 | LIN7B | AC006548.28 | RP11-848P1.9 | HSD17B7 | TSC22D2 |
| ARHGAP12 | FAM53A | POU2AF1 | SEPT7P9 | PHF2 | LINC01480 |
| FCGR2C | IL20RB | ACSBG1 | RP11-172H24.4 | HOXA6 | ELL3 |
| CTC-343N3.1 | WDR1 | ZNF730 | DMRT1 | RPL22L1 | ACE |
| RP11-347I19.8 | LGI4 | KPNA5 | SMAGP | RP5-890O3.9 | SLC35E4 |
| CALCB | AC098614.2 | FKBP1C | FAM35A | ZNF75D | FAM104B |
| CTXN1 | RP11-111M22.2 | RP11-135F9.3 | MCF2L | RP11-367J7.3 | POLRMTP1 |
| SEC23A | VLDLR | VAT1 | KLHL22 | SEPHS1 | CTD-2020K17.1 |
| ZNF22 | EDC4 | SHMT2 | WDFY1 | EPB41L2 | RPL12P28 |
| CTSK | DUSP28 | GPANK1 | KDM5D | AC020571.3 | CKS1BP3 |
| ZNF473 | KIAA0513 | MICU3 | TRIM32 | SLC26A10 | RP11-452F19.3 |
| EGFL8 | AIP | GMDS | HELQ | IGLV5-45 | RP11-158I13.2 |
| GPR63 | BPGM | RP11-862L9.3 | BTG3 | KATNAL1 | TMEM206 |
| LINC01296 | TELO2 | ZNF137P | ERC1 | CTD-2373J6.2 | RP11-17P16.1 |
| DENND5A | ENOX2 | VAMP1 | GOLGA2 | REXO2 | GAS5-AS1 |
| IQCJ-SCHIP1 | MIDN | GMPSP1 | AC090957.2 | FOXC1 | SLC2A8 |
| RP11-542C16.2 | MT1X | NAALADL2 | ADAM10 | ADAMTSL4 | CLDN12 |
| MIR600HG | RP11-50C13.1 | TIGD2 | PRKD3 | MSANTD2 | C16orf62 |
| RP11-461A8.4 | PDE5A | SRP9P1 | HOMER3 | GGT1 | HES6 |
| WFDC21P | LINC01024 | RP11-158K1.3 | RAB27A | SCML2 | SLC25A53 |
| RP11-475C16.1 | PSMF1 | NFYB | TOMM7 | RP11-495P10.10 | KLHL17 |
| RBM8B | ZNF772 | ASPH | RP11-864N7.2 | VIL1 | IL2RA |
| ATG2A | MIS12 | PAK4 | GGACT | KIFC1 | SLC7A7 |
| RP11-474I16.8 | AIM1 | PDE4D | RCAN1 | POTEG | HIST2H3D |
| PPP2R1A | PABPC1P7 | HIST1H2BK | CTB-55O6.12 | RALGAPA2 | FBXW8 |
| RPS6KA5 | POLR1A | RP11-147L13.13 | ACADSB | CBX3P9 | CAMSAP2 |
| ATP7A | CTD-2267D19.3 | RP11-162G10.1 | REM2 | SUV420H2 | HMGB2 |
| ZNF561-AS1 | CTC-523E23.11 | LA16c-306E5.2 | EIF2AK3 | PDIA3P1 | STAT2 |
| RP11-393I2.4 | RP11-465L10.10 | AP2A2 | FYN | DENND5B | RC3H1 |
| RP11-1023L17.1 | CTD-2291D10.4 | DHPS | ASPRV1 | AF001548.5 | RASAL1 |
| AC073869.20 | RP11-29H23.6 | LAMB4 | RP11-399J13.2 | RP11-307C12.12 | RGS12 |
| RP11-155G14.6 | RP11-203L2.3 | ASS1P12 | MROH6 | XYLT1 | CCNY |
| HIST1H2AG | PLEKHG4B | EEFSEC | LTBP4 | IKBKE | CTC-444N24.6 |
| ACOT7 | GS1-114I9.1 | HMGN2P5 | RP11-631N16.4 | RP11-422P24.10 | RP11-881M11.4 |
| SIPA1L1 | Sep-07 | RP11-927P21.5 | AC079610.2 | FBXL14 | KIAA1551 |
| YEATS2-AS1 | LINC00936 | TRAM2-AS1 | LINC00630 | ANGPT2 | MLLT4 |
| RP11-229E13.4 | PCGF6 | MATR3 | SF1 | FAM214B | AC129778.2 |
| KIAA1107 | KIF20B | IL12RB1 | IER5 | ARL8A | GTF2H4 |
| VGLL4 | RPGRIP1L | CACNA2D1 | EHD3 | C5orf30 | ANKRD16 |
| RGPD2 | CTC-351M12.1 | EFNB1 | LINC00672 | FBXO27 | ITFG1 |
| C11orf42 | MRFAP1L1 | HMGN4 | THG1L | PPIP5K1 | ATXN7L1 |
| TMX4 | MGRN1 | ABHD4 | FLT3LG | ELOVL7 | PRX |
| TP53INP1 | ZEB2-AS1 | RP11-2E11.9 | AC020743.2 | TNS3 | RP11-46H11.12 |
| RP11-650L12.2 | LL22NC03-N14H11.1 | KRT8P12 | UQCRFS1P1 | RP11-359J14.2 | CLPTM1L |
| RP11-104F15.9 | ALLC | ASAP1 | NAP1L1 | PBX3 | FBXO46 |
| RP11-705O1.8 | DDX26B | RHEBL1 | CH507-513H4.4 | CH507-513H4.6 | CH507-513H4.3 |
| IGLJ3 | MVP | GPT2 | GOLGA2P10 | FAM50A | RP1-292B18.1 |
| RP11-88E10.4 | CASC3 | SNRPGP2 | KBTBD2 | RP11-264E20.1 | AZIN1 |
| RP11-226L15.5 | FBXW4 | AL162151.3 | RP4-669P10.19 | RP11-638I2.6 | WDR60 |
| HCG18 | RP11-552M11.4 | RP11-142C4.6 | UBE2S | RGPD3 | PCDHGB1 |
| CDC42EP2 | ZNF28 | TVP23A | RP11-35G9.3 | ADAM9 | GDI1 |
| CTD-2116N17.1 | MTHFSD | CUEDC2 | GABPB1 | RP11-1035H13.3 | RP5-882C2.2 |
| PDCD4-AS1 | SLC2A13 | SOS1-IT1 | EXOC2 | Sep-01 | SOX2 |
| FAM43A | AC016700.5 | RP11-6N17.4 | AKAP7 | ZNF844 | EMC3-AS1 |
| AGTPBP1 | EIF4BP6 | TRIM28 | SMC3 | ADORA2B | B3GALT4 |
| TPM3P9 | IL22RA1 | BACH2 | HNRNPA0 | ZNF629 | PRKD2 |
| RP11-321N4.5 | STMN1 | LINC00163 | PIGM | KLHL11 | PRKCE |
| CUTC | THBS3 | AP3B1 | PHF19 | KLHL7-AS1 | DHX16 |
| TRIM22 | KDM1A | FAHD2B | C9orf64 | RP11-968A15.8 | RP11-893F2.18 |
| KAZALD1 | PKN3 | ATP6V0E2 | EIF5AL1 | HIST2H3C | HIST2H3A |
| RP11-739L10.1 | AC011747.4 | CTC1 | CD5L | ZNF711 | OTUD7A |
| MYPOP | RP11-798M19.6 | PRH1-PRR4 | RP4-635A23.6 | RP11-443B20.1 | RPL18AP3 |
| PTP4A3 | TDRD3 | AP2B1 | PHLDA1 | PIK3AP1 | POLR2J4 |
| SLC25A45 | ATXN10 | RP11-1246C19.1 | RP11-484K9.4 | OTUD1 | RCAN2 |
| ATP2A3 | RP11-830F9.6 | LL0XNC01-237H1.2 | KIAA1614-AS1 | PDZD8 | PLXNA1 |
| KCP | WRAP73 | DYNC2H1 | RPSAP15 | SLC25A27 | BUB3 |
| MTERF2 | LRWD1 | ZC3HC1 | FITM1 | SAFB | DACT3 |
| RP11-180M15.7 | TTC5 | PPP1R16A | TPT1P9 | TMEM136 | CXADR |
| AL513523.2 | RN7SKP80 | GNAS-AS1 | RP11-187C18.3 | RB1CC1 | PFAS |
| RAB26 | EFCAB2 | RP11-1191J2.5 | STEAP1B | RP11-330H6.6 | RP11-441O15.3 |
| RP11-211G3.2 | RPP30 | RYK | MATIA | SAP30L-AS1 | ABLIM2 |
| PHF8 | CDCA2 | XYLB | GLI4 | PGAP1 | KIAA1324 |
| CHFR | BNIP3L | SNW1 | USP49 | CWF19L2 | AP000251.3 |
| ZNF513 | PAPD7 | KCNJ2 | AGAP3 | RP11-111F5.2 | TTC8 |
| SMG1P4 | SLCO5A1 | TXK | CADM1 | AC007389.3 | AC007386.2 |
| TOP3BP1 | MEX3C | RP11-403I13.5 | RP1-41C23.2 | FAM65B | POLH |
| YPEL3 | POLM | HCFC2 | CARD6 | AEBP1 | CYSTM1 |
| COPS4 | RNASEH2B | ZNF407 | CLDND1 | TCEAL1 | LEF1-AS1 |
| SLC35E2 | CD81 | RP11-57H14.4 | TMEM117 | CORO2A | RP4-765C7.2 |
| HAUS5 | SLC12A2 | PIGN | CD52 | RNF220 | C9orf163 |
| INTS4 | BEND4 | TCTN1 | RP3-424M6.4 | CNNM2 | MLF2 |
| IL2RG | CRLF1 | PCNXL4 | PABPC3 | KLHL24 | BRICD5 |
| THAP3 | RP11-530A18.1 | RBKS | SUGCT | RP11-343H5.4 | RP1-150O5.3 |
| BFSP2-AS1 | C7orf60 | SIRT1 | SAV1 | TAF6 | KMT2E |
| GDPD1 | ZNF804A | WWP2 | MCPH1-AS1 | NR1I3 | TRPC1 |
| C17orf62 | NT5DC2 | EMC3 | PCGF2 | TTLL4 | RP11-262H14.4 |
| C11orf95 | EIF4E3 | CDIP1 | AC015849.16 | RP9 | BTAF1 |
| AC093673.5 | FN3K | SNORD17 | RP11-65L3.3 | INSIG2 | SLC44A2 |
| RITA1 | NRDE2 | DCBLD1 | RP11-146F11.1 | AC138035.3 | NUP50-AS1 |
| FCHSD1 | TRIM24 | RP11-594N15.3 | CPNE2 | ADAM1A | RP11-15F12.1 |
| AHCYL2 | STXBP5 | PDZK1 | RXRB | FIZ1 | HERPUD1 |
| MAGI2 | PRR12 | SLFN5 | HCLS1 | ETV4 | PGAM1 |
| AP001347.6 | POLE4 | SPAG4 | CDKN2AIP | RP11-652L8.4 | PRICKLE3 |
| RP11-182L21.6 | TES | WSB2 | TBL1XR1 | MORN3 | HOXA7 |
| PWP1 | SLC27A2 | FAM213A | RP1-34B20.21 | RP11-380I10.4 | MARCH5 |
| ATAD1 | CTC-281F24.3 | CRB2 | FRMD6-AS1 | CDKN2C | TMEM185B |
| RP11-29G8.3 | NTAN1 | CRMP1 | RP3-395M20.9 | RP11-295H24.4 | MCTP2 |
| RP11-253E3.3 | RBBP4 | CCDC77 | SUMO1P3 | PI4K2A | RP11-20I23.8 |
| LANCL1 | BMP2K | PAQR3 | FXYD1 | INCENP | L3MBTL1 |
| TAB3 | TM7SF3 | NUDCD3 | CCNJ | HJURP | CHDH |
| CTC-425O23.2 | ELAC2 | RP1-241P17.4 | PEMT | CCDC168 | ITGA2 |
| AC090804.1 | AC009542.2 | CAPN5 | TOM1L2 | RP11-5O17.1 | FCHO1 |
| UBQLN2 | P2RX2 | HIP1 | ZSWIM7 | RP11-713M15.2 | CD3E |
| PPP1R14D | LINC00894 | Z83844.1 | SNRPGP10 | ABCG1 | BLOC1S2 |
| SCPEP1 | CTD-2287O16.1 | PDPK2P | ZSWIM8 | MAPK8 | ZNF555 |
| TNKS2 | RP11-932O9.7 | ZNF157 | FLNB | CSNK1G2 | FANCI |
| TRIM7 | PDCL3P5 | RBM12B-AS1 | ARHGEF26 | GPR31 | DYNC1LI1 |
| SOGA3 | AC109486.1 | BAIAP3 | RPS10-NUDT3 | EML4 | CEP97 |
| MAGEF1 | RP5-1136G13.2 | RP11-651P23.4 | BMPR2 | LINC01560 | ZXDA |
| COMMD3-BMI1 | CTC-523E23.5 | ZFAND2A | WDR86 | GRIN2C | TMEM65 |
| FOXD4L1 | CTD-2227E11.1 | ITPR1 | C5orf42 | TUBA3D | CH507-396I9.7 |
| RP11-139H15.5 | RHOH | FBN1 | MFSD2A | PARN | HILPDA |
| LCLAT1 | AC058791.1 | SEMA4G | SLC35A4 | MAGEH1 | ANKHD1 |
| ACOX3 | C1orf56 | GTF2IP4 | LINC01144 | PDS5B | SGOL2 |
| RP11-138C9.1 | GABRB2 | CYLD | C7orf26 | IGKV3D-7 | ST13 |
| ZNF615 | RP5-894A10.2 | OTULIN | ZNF10 | SIGIRR | HIST2H2AC |
| EFTUD1 | RP11-324E6.10 | TBC1D27 | PPL | RP11-566K11.5 | RNPC3 |
| GTPBP3 | CSTF2T | NCOA5 | RP3-508I15.18 | AC016831.7 | HARBI1 |
| RTKN2 | BCL3 | ARNTL | IL24 | LAX1 | SAFB2 |
| RP1-47M23.3 | RP11-61K9.3 | RP5-1159O4.1 | C2 | RP13-608F4.5 | GLCCI1 |
| SNX8 | SPI1 | IGF2BP1 | CENPV | AC010547.9 | FIP1L1 |
| BEND3 | RP11-22N19.2 | RP11-53O19.1 | PCGF7P | AC021224.1 | KIAA1586 |
| RP5-961K14.1 | RP11-412D9.4 | SH3BGRL3 | ZNF384 | CNPY2 | SLC6A9 |
| ZNF878 | EHMT2 | SMAP1 | PRCD | CFDP1 | MFN2 |
| XKR7 | HERC3 | FLII | DEDD2 | SH3BP1 | ZNF496 |
| RP11-490B18.6 | PITX1 | CENPW | ZNF580 | RP11-320L11.2 | TLE4 |
| RPL23AP97 | DGKQ | NELFA | CTB-119C2.1 | ZNF605 | FBXO32 |
| RPL15P3 | HPS6 | VAV3-AS1 | ZADH2 | AOX3P | RP11-61F12.1 |
| CASC4 | SUGP1 | PPP1R10 | MAPK8IP3 | RAD52 | RPLP1P6 |
| NCOR1 | PPP1R2 | HPS1 | RAB43P1 | OR6L2P | ZNF343 |
| FAM120B | KCTD7 | ASMTL | ASMTL | SNHG5 | GCN1 |
| DSE | TMSB10 | ZNF529-AS1 | ACTR1A | ZNRD1 | KIAA1683 |
| LRRC37A16P | DENND2C | RP5-1126H10.2 | GMEB1 | TMEM151B | FAM193A |
| THOC3 | ADRBK1 | ZNF321P | CDK2 | TFIP11 | TMEM133 |
| AC008522.1 | UTP14A | PKMYT1 | AC002310.14 | SUMO2P17 | ACMSD |
| ASB16-AS1 | LINC00282 | AC092835.2 | TMCC2 | QDPR | KIF21B |
| MIR3142HG | STXBP4 | SAXO2 | RP11-196G11.3 | RP11-83J16.3 | TOX |
| LINC01096 | CTDSP2 | IL17RB | ARRDC1-AS1 | BAZ2A | TTC32 |
| RP13-616I3.1 | AC004540.4 | ITGB3 | SAMD12 | EPS8L1 | ALDH3B1 |
| FCRL4 | AKTIP | GPSM3 | LINC01206 | ATXN7L2 | AMBRA1 |
| CTD-2647L4.4 | PTCH1 | KATNA1 | TNFRSF13C | DNASE1 | DUXAP9 |
| SPTBN4 | RP11-326I11.3 | CLOCK | ZNF623 | KAT6A | STOML1 |
| RP11-649A18.12 | ZNF888 | RP11-114O18.1 | PRKCQ-AS1 | INPP5D | RP11-44F14.7 |
| ZNF140 | AK9 | CITED2 | NCOA2 | BX255923.2 | ZFP92 |
| SERPINH1 | SEC31A | CLIP3 | ZNF451 | TTLL13P | OPRD1 |
| EMILIN2 | RP11-177C12.1 | AKNA | RP11-33O4.1 | RNU12 | AC005776.1 |
| CTD-2527121.15 | RPS6KA3 | STX18-AS1 | IGKV1D-8 | RSAD1 | IL10RB-AS1 |
| INTS6-AS1 | UBE2D1 | RP1-28H20.3 | GPATCH2 | RP1-8B1.4 | SLC4A11 |
| ATAT1 | RHBDF1 | PRKCZ | IFI27L1 | CLPP | KHDRBS3 |
| HNRNPA3P12 | SCAND2P | IGKV3-20 | RIPK3 | EZR | UBE2F-SCLY |
| RP11-351D16.3 | RP11-168J18.6 | CTB-25B13.12 | GOLGA8Q | OCRL | RP3-399L15.3 |
| CASC5 | RASGRF2 | PLAGL1 | CTSF | ZNF226 | LINC01431 |
| GNRHR2 | MAGED4B | LAPTM4A | PEX10 | CTC-564N23.2 | SLC27A3 |
| ZNF37BP | RP11-344P13.6 | GK | KCNH4 | RFX2 | AP1S2 |
| BACE1-AS | RP1-179N16.6 | SAMD15 | AKAP8L | SNHG14 | AC007969.5 |
| CTD-3214H19.6 | GNG3 | E2F6 | ASB12 | FUOM | SKI |
| MOSPD3 | GTF3C1 | RP11-271K21.12 | LINC00324 | PAF1 | LRRC37A9P |
| C14orf28 | SEMA4F | RP11-135J2.3 | RP11-596C23.2 | HCFC1 | PJA2 |
| RP11-173A8.2 | NBEA | MAP2K4 | NLRC3 | ADM | ZCCHC3 |
| DUSP8P5 | ERCC4 | ZBTB1 | COLGALT2 | RP5-942I16.1 | ARFGAP3 |
| YEATS2 | DNAJC4 | RP11-588H23.3 | CASP7 | SRI | RP11-479O9.4 |
| AC068580.6 | RFWD3 | LINC00237 | VPS13B | CTC-575D19.1 | ARL13B |
| LA16c-3G11.7 | FANCG | EIF3A | MCM4 | TSC22D3 | HUS1 |
| ARHGAP11A | RCHY1 | TMEM163 | TIGD1 | LINC01087 | KLF12 |
| CCDC24 | WIZ | MTRNR2L8 | PSMB8-AS1 | RP11-134N1.2 | PLEKHM3 |
| HIST2H2AA4 | PHOSPHO2 | PARP1 | VEGFB | RP11-746P2.3 | RP11-722G7.1 |
| SPDYE2 | SNRNP35 | C9orf24 | RP11-68L18.1 | ZNF432 | TRMT2A |
| IGHV4-59 | N4BP2L1 | AC005339.2 | TIFA | DDX50 | LRCH3 |
| RP11-430B1.2 | RP11-211G3.3 | RP11-15H20.6 | CUL1 | ZNF670-ZNF695 | TSHZ1 |
| TRANK1 | EID2B | MCM3AP | P2RY8 | P2RY8 | WDR76 |
| COX6A1P2 | WDR13 | ZBTB18 | PLK1 | ZNF518B | CHUK |
| RP4-568B10.1 | AC006547.13 | ZNF598 | KIAA1147 | PRPF31 | HAPLN3 |
| MAST3 | RP11-332H14.2 | CTD-2342N23.3 | ZFAND5 | ZRANB1 | ZNF271P |
| ERICH6 | GZMM | RP11-325K4.2 | RP11-403P17.6 | RP11-204L24.2 | SNAP47 |
| TSHZ2 | KCNK15-AS1 | TAS1R3 | UROS | THRIL | MBD4 |
| TAS2R12 | RP11-91P24.6 | RP11-475I24.1 | RRM1 | ENO1P1 | STARD13 |
| HSPA8P3 | CHIC1 | FAM45B | TTL | PHKG1 | RELA |
| RP11-53B2.2 | PCDHB12 | ZNF749 | FUT8-AS1 | PLEKHG1 | ANO8 |
| GAPT | HTATSF1 | ARHGDIA | VSIG10L | MAGED2 | RP11-863P13.4 |
| ZNF20 | AC079630.4 | NOC3L | SIN3B | RP11-91G21.1 | SIDT1 |
| ZNF133 | RP13-103211.7 | KIF2A | AP002884.2 | RP4-800J21.3 | C1orf50 |
| HNRNPA1L2 | RHOC | RP11-244O19.1 | E2F2 | CCL4L2 | MKI67 |
| RP11-1378G18.1 | ZNF416 | SLC24A4 | CASC2 | STYK1 | RP11-298I3.5 |
| LDHAP4 | RALGAPA1 | DNMT3A | LINC00294 | LMLN | LINC01011 |
| DGKZ | RP11-298J20.4 | DMAP1 | CAMLG | ZNF552 | RPS28P7 |
| AFG3L2P1 | ARL11 | HMGN2P4 | NPIPA3 | RP11-171I2.5 | ZNF441 |
| MIR181A1HG | BAHD1 | FAM27C | TTK | ZFAND3 | RP11-408O19.5 |
| AC002398.13 | MED22 | ALPK1 | IDE | RN7SL738P | RN7SL172P |
| CTD-2530N21.4 | POU5F1 | HNRNPKP4 | POU2F1 | COL5A2 | RP11-110I1.13 |
| RPL23AP2 | ABLIM1 | ITGA4 | CTC-559E9.5 | ZBED5 | TRMO |
| OSBPL9 | SIPA1L3 | MAPKAPK2 | HLTF | RP11-643M14.1 | DIAPH2 |
| IFT52 | CECR2 | GPR137 | MCMBP | KCNE5 | CTB-31O20.2 |
| ADD3 | HMCES | ZBTB5 | PTBP1 | TRPT1 | LRRC8E |
| TMEM235 | TROAP | MCIDAS | RP11-850A17.1 | RP11-192H23.7 | MAP2K4P1 |
| TFAP4 | LINC01089 | TNFAIP3 | GNB5 | CMB9-55F22.1 | RDH16 |
| EMC2 | C12orf57 | SCRN3 | SCAMP1 | AP001619.3 | IPO9-AS1 |
| FAUP1 | ECM1 | ZNF331 | KCNIP2 | SSUH2 | TMPOP2 |
| EEF1A1 | CORO6 | UTY | NDUFB8 | RUNDC3A | AC079250.1 |
| CTC-444N24.13 | RP11-359K18.4 | KLHL26 | CERKL | RNF20 | DSTN |
| PDK2 | RP11-567J20.3 | LGMNP1 | AC004076.5 | FUNDC2 | TONSL |
| ATP7B | RP13-554M15.5 | RP11-195F19.5 | MDFIC | PLXND1 | KMT2A |
| HNRNPCP2 | ANKRD44 | DDX24 | RP11-75L1.1 | LINC00426 | SNHG6 |
| RP11-428J1.5 | ZER1 | RABEP1 | NR1D2 | COL5A1 | INO80C |
| RP11-727F15.11 | CDC20P1 | SEC24D | GJB7 | TEX29 | ZNF624 |
| LIG1 | KCNQ5-IT1 | EXTL3-AS1 | FKRP | ATP1B2 | CTC-205M6.1 |
| CTB-13F3.1 | FOXN3 | RP11-670E13.6 | C11orf30 | NPM3 | SP9 |
| ELF2 | GRAP | PKIG | AC092159.2 | LA16c-60H5.7 | AP4M1 |
| LRRC8A | ACSBG2 | ABCD3 | RP11-690I21.1 | LRMP | RP11-588G21.1 |
| CTD-2555O16.2 | NT5C | MED31 | TBL1X | TRDMT1 | TBC1D5 |
| ZNF277 | IFT27 | SEC23IP | RP11-353N14.1 | HELZ2 | RP11-166P13.3 |
| FAAH | ZNF707 | ZRSR1 | PRRC2B | BMP6P1 | PELP1 |
| RP4-798A10.7 | JUN | RP11-726G1.1 | INTS10 | RP11-540A21.2 | CAMK1D |
| OBFC1 | RP1-172N19.5 | GTF2IRD2 | RORA | TIA1 | CTD-2339L15.3 |
| ERLIN1 | LINC01483 | HNRNPCP7 | LTBP3 | SPCS2P4 | LRRIQ3 |
| RP11-640L9.1 | NUP62 | CTA-373H7.7 | CTD-2165H16.4 | IRF2BP1 | AHSA1 |
| LIG4 | LACTB | CDYL | AC006994.2 | AC064850.4 | RNF31 |
| RP11-706O15.1 | GALNT8 | MYLK4 | RAB43 | TRIM41 | AC096579.13 |
| C9orf131 | CMTM1 | WDR11 | LSM14B | LIMD2 | PARPBP |
| AK6 | DOPEY1 | CLMN | CRADD | PTPRO | NCKAP1L |
| RP11-375N15.2 | CA11 | PTMAP5 | CTD-2349P21.5 | RP11-368I7.6 | RP11-1228E12.1 |
| RP11-159D12.6 | RP11-677M14.7 | RPS11P5 | RP11-315O6.1 | RP11-793H13.3 | TMX2-CTNND1 |
| GOLGA6L10 | FAM98A | TRIM2 | RP11-424N24.2 | KB-1980E6.3 | AL022393.9 |
| FNTB | TMLHE-AS1 | ADO | DLL3 | CTC-559E9.8 | ZNF248 |
| FKBP1A | PNRC1 | CCNG2 | SKA2 | GAL3ST4 | SLC52A1 |
| AC159540.2 | WBP2 | DOK3 | PRDM11 | RP4-781K5.2 | SLC44A1 |
| RP11-139H15.7 | RASAL3 | IL4R | TSPAN9 | RP11-167N5.5 | RP11-114M5.1 |
| DTX4 | TRIM26 | HIST1H2BE | OGDHL | DLG1 | RP11-379K17.4 |
| HNRNPUL1 | DDX46 | ZBTB8OS | TAF7 | E2F1 | VPS35 |
| RP11-227G15.12 | IGHVII-22-1 | APBB1IP | WDR41 | TUBB | CH17-264B6.3 |
| PAICSP1 | RP5-994D16.9 | LINC01250 | RACGAP1 | CTC-332L22.1 | GPR153 |
| ADD3-AS1 | NUTM2B-AS1 | LIX1 | F8A3 | RP11-794P6.1 | MAP10 |
| SOCS4 | RP4-758J18.2 | FBRS | AC004381.6 | MRPL43 | LINC01305 |
| SLC13A5 | FAM63B | LINC00235 | HIBCH | AC009120.6 | CTDSPL2 |
| RPL32P3 | PUDP | ANKRD12 | PGP | METTL7B | RP13-494C23.1 |
| SMARCA5 | RP1-60O19.1 | CHRNG | NDUFS4 | SFMBT2 | PGK1 |
| NISCH | RP11-4L24.4 | ELMO2 | RYBP | DCTN1 | SNX11 |
| RP11-345P4.9 | TSNARE1 | PTCHD2 | TEC | CTD-2527I21.14 | SEC63P1 |
| MYBL2 | FAM107B | AC012358.4 | AC007041.2 | IQCB1 | YWHAZP3 |
| BTBD10 | RIPK1 | PIAS1 | MKL1 | NBPF2P | ZNF16 |
| RP11-505K9.4 | CTD-2033A16.2 | RPS2P46 | XRCC6P2 | RP11-252K23.2 | PPP1R12C |
| XXbac- | PRKAR1A | BBS1 | MBD6 | RP11-411B10.2 | PDLIM1 |
| BPG27H4.8 | | | | | |
| AC097662.2 | GNMT | PPP2CA | SOX7 | RP11-93H24.3 | HMGB3P22 |
| SCNN1G | PPM1L | TSSC1-IT1 | SCMH1 | FAM153C | LDLRAD2 |
| RP11-525A16.4 | RP11-36B15.1 | CTD-2116N20.1 | CTD-2162K18.3 | SLC9B1 | GNG5 |
| RP11-380B4.3 | ZNF274 | SYNPO2 | CXorf65 | RP1-265C24.8 | LMO7-AS1 |
| AC007191.4 | MOB3B | STAM | RP11-727A23.4 | SUMO4 | RP4-742C19.13 |
| RPL23AP65 | RP11-293A21.1 | SENP7 | RP13-766D20.2 | DRAM2 | GTF2H2B |
| ITPKC | FAM3B | AC097461.4 | RP11-11M20.4 | RP11-415J8.3 | IFT172 |
| CKAP2L | KIF4A | CTB-58E17.1 | SART1 | AC000068.5 | RP11-454F8.2 |
| PRH1 | SMAD1 | FIGNL1 | GPD1 | LEMD3 | MYL6 |
| RPS12 | AC078942.1 | KRTAP5-AS1 | SLC45A4 | NDUFA3 | SIMC1 |
| FEM1A | TNRC6A | PPP1R3E | RP11-686O6.2 | CCDC142 | METTL4 |
| MDM4 | AMBP | PSMD2 | ASNS | ZNF329 | HIST1H2AB |
| SYNPO | CEP164 | AKAP2 | GSTO1 | RP11-553L6.3 | TMEM177 |
| TMEM263 | AC006449.2 | EIF3CL | RP11-823P9.3 | N4BP2L2 | MAGED4 |
| ZNF428 | KRBA1 | MSL3 | ATP6V1E2 | RHOQP1 | RP1-203P18.1 |
| MAGOHB | IRAK3 | SLC4A8 | LL0XNC01-7P3.1 | ANKRD52 | ZNF510 |
| RP11-1334A24.6 | MAFK | ANKRD20A11P | PEX14 | TKFC | FOXO3B |
| RP11-707M3.3 | TIAL1 | SKIL | ACSL1 | IKBKAP | SLC38A4 |
| TDRD7 | GPRIN1 | WBP1L | KCTD9P2 | TRAC | FPGT-TNNI3K |
| CHSY1 | RP11-78J21.7 | RP5-968P14.2 | ELAC1 | NAB2 | BCL10 |
| FUT1 | KCTD6 | ZNF610 | RP11-456K23.1 | DAXX | CEP63 |
| LINC01578 | C21orf2 | TNNT1 | RBM17 | LRP8 | PHC2 |
| AKT2 | TRIM36 | EEF1A1P5 | SP2 | CBLL1 | AMPD3 |
| DTX1 | ZSCAN20 | PXK | FANCL | SC22CB-1D7.1 | RP11-156P1.3 |
| SH3BGRL | RPL12P37 | MORF4L1 | RP11-603J24.7 | AC004156.3 | RP11-467D6.1 |
| UBE3D | CSNK1G3 | SUFU | RP11-334C17.3 | RHOBTB3 | DLEU2_6 |
| HTRA3 | TGIF1 | CTC-559E9.4 | FITM2 | INTS1 | SOX6 |
| AC005307.3 | ARHGEF3 | PSTPIP1 | HIST2H2BC | FBXO28 | C9orf47 |
| GSTCD | ZMAT1 | ZGPAT | CIRBP | RP11-446E9.1 | WDR48 |
| CCNT2-AS1 | RP3-406P24.4 | RNF170 | PI4K2B | RP11-230C9.2 | TAGLN2P1 |
| LIPA | RP11-53B2.1 | PLA2G16 | APTR | NSFL1C | AC027601.1 |
| STK4-AS1 | LINC-PINT | FAM86HP | RP1-257A7.5 | HIST1H4C | PLEKHA8 |
| INTS9 | RNF6 | CTD-2619J13.19 | HCG25 | RP11-667K14.4 | LYPD6B |
| PER2 | AXDND1 | PELO | ZNF350 | HIST1H2BO | DCAF12 |
| PEX3 | RP11-134D3.1 | EML2-AS1 | CTD-2371O3.3 | HIPK2 | U62631.5 |
| KRCC1 | ZACN | ZNF738 | IL17D | SART3 | RIMS2 |
| SYNE4 | LINC01012 | WDR47 | RP11-325I22.3 | STAMBP | RP11-96K19.4 |
| AP001462.6 | LA16c-329F2.2 | RP11-763B22.4 | FGF11 | KLF10 | ZNF621 |
| RWDD2A | APOF | PSMC3 | RP5-1182A14.7 | ST13P5 | PARP11 |
| CTC-325H20.4 | C10orf111 | GS1-44D20.1 | DLEU1_2 | ZNF131 | RP11-61J19.5 |
| SKIV2L | LL22NC03-27C5.1 | KIAA0101 | MCM10 | ZNF227 | SLF1 |
| AP001258.4 | BRAT1 | ZNF688 | AC002550.5 | RP11-634H22.1 | VPS28 |
| GOLGA8O | AP000936.1 | JPX_1 | HIST1H2APS4 | RP11-429G19.3 | AC083949.1 |
| AC012363.7 | RP4-669K10.8 | KRT8P46 | IGHG2 | CENPN | FZD6 |
| RP11-573M3.3 | RP11-818O24.3 | VKORC1 | MTG1 | LDB3 | TNS4 |
| RPL41 | TJP3 | RP11-863K10.4 | MINA | RP11-178H8.7 | POTEH |
| TDP1 | TRIM37 | PPIG | CIDECP | CTD-2256P15.2 | SNX32 |
| RP11-111F5.3 | DHX32 | RPL13A | LINC01058 | LMNB2 | SLC7A6OS |
| RP4-555D20.2 | RP11-20I23.10 | RP13-650J16.1 | GDAP2 | RP11-296A16.1 | AC092641.2 |
| DGCR2 | RBMXP2 | ST7 | CCDC102B | TBX19 | CTD-2184D3.6 |
| MYLK-AS1 | ZNF581 | ZNF799 | SLC25A25 | WIPF2 | AC112497.1 |
| HIST1H3A | C4orf33 | RHOA-IT1 | ZNF252P-AS1 | YTHDC1 | RP11-166B2.1 |
| SLC35G1 | LINC00115 | RP11-313P13.5 | UBR4 | C15orf41 | PLA2G6 |
| PPP1R15A | HIST1H3I | CTD-2561J22.2 | GNPDA2 | ELP5 | ZFX |
| RP11-481J2.3 | RP11-401L13.4 | DCAF13 | FLJ37453 | NEK7 | TCEANC |
| CFL1 | HEATR5A | SND1-IT1 | DNAL4 | ZNF710 | RP11-140I16.3 |
| TCEB2 | RP11-746M1.1 | CIT | RPL37 | AC010733.5 | MUM1 |
| TOP2A | ANKRD30BL | KB-1608C10.2 | GALNS | EMX1 | RPS4XP16 |
| C7orf55-LUC7L2 | CBX5 | MAN1A2 | KCNG3 | AARS | PSMD1 |
| C22orf23 | MEX3D | RASA2 | AC092798.2 | BORCS5 | TBCB |
| RP11-407G23.5 | PACSIN1 | RP11-216B9.6 | TUBBP1 | ZFP62 | CTD-3105H18.16 |
| POLRMT | POM121B | TMEM234 | AC113404.1 | PBRM1 | RALY |
| FNIP2 | CTD-2293H3.1 | GNAI2 | ERCC2 | POP1 | FOXK2 |
| CTD-253719.18 | NAGS | CTD-2161E19.1 | SNUPN | MESDC1 | TMEM182 |
| RP11-181G12.2 | ZNF512 | SCARF1 | PARD6G-AS1 | Metazoa_SRP | AC012358.7 |
| SIPA1 | ZNF322 | RP11-261P13.5 | GPX3 | TRAK2 | ZMYND8 |
| NRGN | CAPS2 | PCDHB10 | RP11-338I21.1 | RAD21 | CTC-428H11.2 |
| MERTK | AK7 | MTMR4 | COMMD3 | HOMER1 | FRA10AC1 |
| RP11-958N24.2 | BEST4 | CTR9 | RPL26P30 | LEO1 | RP11-463O9.9 |
| PSTK | CTD-2006C1.10 | AC073043.1 | TRIM21 | KIAA0319 | DLX4 |
| TMPO-AS1 | KIAA0586 | ADAMTSL4-AS1 | KRT8 | MCPH1 | TNFSF10 |
| RP11-582J16.4 | PHACTR2 | LSM11 | AC084219.4 | RASAL2 | RP11-134G8.6 |
| DGKA | ECI2 | CTC-436K13.6 | KCNH1 | RP11-398K22.12 | TRPM2 |
| RP11-85G18.6 | CKAP5 | LDLRAD4 | C14orf169 | DHX37 | IGF2BP3 |
| SHF | RP11-29B2.6 | RP11-417L19.6 | LINC01497 | RP1-92O14.6 | NBPF8 |
| CTD-2550O8.5 | ZKSCAN8 | EPC2 | DHTKD1 | ABCA2 | SPRY3 |
| SPRY3 | HIPK1 | UXT-AS1 | PKD1P1 | EAF2 | RP11-714G18.1 |
| DECR1 | RP11-677M14.2 | ALKBH8 | WDR33 | ATXN7L3 | AC005546.2 |
| UGDH | PLIN2 | AC011290.5 | RNF111 | CTBP1-AS | MIB1 |
| RP11-195C7.1 | RP11-16N11.2 | LMO4 | HIRIP3 | CTD-2033C11.1 | MDK |
| HLA-DOA | FOXD4L6 | BRD9 | ARL1 | PRR3 | UBE2Q2P2 |
| CKLF-CMTM1 | HNRNPA1P59 | ING4 | NUP93 | ZNF175 | NUP188 |
| AAK1 | AC010900.2 | GLRX5 | RP11-750H9.5 | ZNF740 | TMEM55A |
| APPL2 | HIST1H2BG | USP39 | MAP3K5 | MFAP4 | AC005062.2 |
| MAFG-AS1 | RP11-32B5.1 | ZNF213 | SNRPN | RP11-287D1.2 | GPR156 |
| TRAFD1 | TBC1D3D | PRRT1 | ITGB5 | ADNP2 | FBXO10 |
| AUTS2 | TRMT112 | ANKRD33B | SLC47A2 | DIP2B | RP11-54D18.4 |
| RP11-356M20.2 | AP001437.1 | ESYT2 | L34079.4 | UBR5 | RP11-81A1.6 |
| GTF2E2 | PRICKLE1 | ANKAR | SUSD5 | CDRT15 | RP11-341N2.1 |
| LNX1 | C10orf88 | TBPL1 | HSPBAP1 | ASF1A | TRMT6 |
| PTMA | PKMP3 | RFX3-AS1 | AC063976.7 | BGLAP | H3F3A |
| RP11-242D8.1 | RGMA | RP11-301O19.1 | EXT2 | RP11-571M6.17 | MRPL55 |
| SMAD5 | SPDYE8P | CAPN14 | STS | COG4 | TAF1C |
| PTPDC1 | TNXB | CKMT2-AS1 | RN7SL23P | SAMD4B | PIN4 |
| PADI2 | ZNF285B | TRIM38 | GABRR2 | RP11-131L12.2 | FAM173B |
| VWA5A | RMDN2-AS1 | RP11-715J22.4 | HPS3 | ANKRD36 | TRIM44 |
| TEAD3 | RPS2 | RP11-114F3.4 | ITPK1 | EIF1AXP1 | KRI1 |
| DYNC1H1 | RP4-621N11.2 | MAGEB6 | PDE7A | BCR | LRRC7 |
| HIST2H2AB | CGGBP1 | FBXO43 | RP4-536B24.4 | PGM2 | LMNB1 |
| RP11-677M14.3 | AC093484.4 | BLOC1S5-TXNDC5 | AP4S1 | CDK7 | RP11-524F11.1 |
| TBC1D3F | FOXP4 | RP11-792A8.4 | CLK2P1 | NLGN3 | RP11-386I14.4 |
| RP11-552F3.10 | TREX2 | EMILIN1 | TTPAL | NET1 | SOD1 |
| FAM60CP | DLEU2 | CTD-3222D19.4 | LIN54 | RP11-128A17.1 | RPSAP58 |
| RP3-402G11.28 | POMK | HELZ | IGIP | KANTR | ZFP91 |
| EML3 | NXPH4 | VN1R83P | FBXO5 | RPS2P5 | CDON |
| CNIH4 | NPM1P6 | CAP1 | DUBR | HMGB1P1 | EBF4 |
| CNOT2 | CHRNB2 | PAQR8 | LINC01278 | AC007950.2 | RP11-158I9.5 |
| HIST1H2AH | FNDC3B | RP11-154J22.1 | RP11-358L22.3 | DPYD | RP11-449H3.3 |
| DND1 | SCX | HNRNPH3 | RP11-830F9.5 | ITPKB | METTL9 |
| KIF3A | KLF6 | AC016292.3 | PBK | RP11-43N16.4 | CTD-2630F21.1 |
| AC069513.4 | FAM222A | LA16c-431H6.6 | EIF4E2 | BCL9L | DLGAP5 |
| RP11-67K19.3 | PRKAA1 | SNRPA | GIT1 | AMDHD2 | RP11-533E19.7 |
| TSSK1A | FAM220A | SUV39H2 | ZBTB2 | RAB13 | RP4-773N10.4 |
| ATPAF1 | BLOC1S1 | TMEM110 | GLYATL1P1 | ZNF316 | CTNNAL1 |
| ARRDC2 | TESK1 | SPAG9 | ZNF142 | C17orf67 | RP11-53B2.5 |
| PTPN23 | RP4-569M23.2 | LRRCC1 | RP11-5G9.5 | SPATA25 | FKBP9 |
| JAKMIP2 | CCDC12 | KDM2B | TERFI | SLC26A2 | SYF2 |
| IL12A | SERINC4 | CRTC3-AS1 | STK40 | KHDC1 | PCBP2P1 |
| HSDL1 | CPAMD8 | ZFAND1 | RHBDL2 | STAG2 | RP11-65B7.2 |
| ADM5 | RGS1 | WDR34 | RP11-195B21.3 | DPY19L1 | CEBPB-AS1 |
| WDR70 | EBF1 | LDHB | RP11-294J22.7 | HLA-J | ZNF808 |
| ZNF174 | MGST1 | FAM19A2 | ZNFX1 | CTC-457L16.2 | CTC-248O19.1 |
| XRN2 | RP11-283I3.6 | CD58 | MAPK6 | CTC-487M23.5 | HDAC2 |
| SLC7A8 | RP11-147L13.11 | CHD4 | ZNF425 | HDAC9 | FBXW5 |
| HYOU1 | AC009948.5 | HIF1A | PDIA2 | PLEKHA6 | CTD-2532N20.1 |
| F2RL3 | LL0XNC01-116E7.2 | MBNL1-AS1 | ROPN1B | HNRNPR | MATR3 |
| NCBP2-AS1 | KIAA0226L | HIPK3 | NACAP1 | RHEBP2 | RRAGA |
| KCND1 | PURG | CTD-2384B9.1 | GDF11 | C17orf49 | FCMR |
| PATZ1 | DGKG | RP11-181C21.4 | EVC | LUZP1 | H3F3C |
| EP300-AS1 | RNF186 | UBC | PGM1 | CTD-2619J13.16 | CEP295 |
| ZDHHC13 | RP11-356N1.2 | CTD-2201I18.1 | SH3GLB1 | RP11-2C24.9 | CES2 |
| RP11-532M24.1 | UBR1 | NR1H2 | RP11-762I7.5 | AKT1 | AC074117.10 |
| FAM175B | ZNF562 | C18orf65 | CDT1 | RP4-734G22.3 | RP11-540B6.6 |
| RPL7P21 | RP11-78O7.2 | RP4-801G22.2 | RP1-29C18.9 | RP1-45N11.1 | ZNF544 |
| AMH | SNCAIP | LINC00638 | AC083873.4 | NXPE3 | ALOX5 |
| TLR8-AS1 | WBSCR16 | RP11-727A23.5 | NFE2L1 | RAB6B | CRELD1 |
| ACRBP | SOX12 | RP11-51F16.9 | IKZF4 | ARHGEF19 | TMEM135 |
| RP4-612B15.3 | GOT1 | FUBP1 | CKAP2 | GPR61 | ZFP36L1 |
| CATSPER3 | COX10-AS1 | RP11-29H23.7 | BCRP3 | CXorf40B | HOMER2 |
| AC093323.3 | NPIPA1 | KIAA0556 | ARHGEF10 | CMB9-55A18.1 | CTD-2012K14.5 |
| GLT8D2 | RP11-119B16.2 | AF196970.3 | RP11-849F2.5 | RP11-21M24.2 | GZF1 |
| SNHG25 | ENY2 | FAM172A | NUP210L | TCF19 | ILK |
| SDF2L1 | IGLV1-44 | MED17 | RP11-165J3.6 | POLR2I | PCNT |
| PGBD2 | ARFGAP1 | ZNF286B | CTC-301O7.4 | MTERF4 | C2orf68 |
| PPRC1 | NT5DC3 | POC1B-GALNT4 | HHAT | TTC4 | AP000275.65 |
| PET117 | THSD1 | PIK3CD | KIAA0895 | ZNF250 | MAD2L1BP |
| RHEB | HIST1H1B | SLX4 | Sep-06 | RP11-1023L17.2 | NSMAF |
| AC004906.3 | NAMPTP1 | SLC3A2 | AC079922.2 | GREB1 | ARSB |
| HERC4 | MTCP1 | SLC35A1 | ACTBP2 | CTB-109A12.1 | C1orf53 |
| SCAF4 | SFXN2 | RP11-164P12.3 | CTC-398G3.2 | SP1 | RANBP3 |
| PTK7 | EZH2 | RP11-4L24.3 | PARP10 | CENPE | CALCOCO1 |
| MNAT1 | AC020951.1 | CD99L2 | MAP7D1 | CACNA1A | ZBTB14 |
| MEA1 | MTA2 | KDM5A | CNOT4 | RP3-466P17.1 | NPAT |
| RP11-111M22.5 | SATB2 | SCD5 | RP11-384P7.5 | PROSER3 | CSRP2 |
| EDRF1 | JUND | ROM1 | RNVU1-15 | LRP10 | RAF1 |
| HNRNPA3P11 | PPP4R1 | PIH1D2 | HDAC7 | HGH1 | CTD-2639E6.9 |
| RPL12P4 | GAPDHP62 | STK32B | MFAP1 | CTD-2555A7.3 | RP11-10L12.4 |
| EML2 | KLHL31 | RP11-45M22.2 | RP3-469D22.1 | CTC-273B12.10 | HNRNPUL2-BSCL2 |
| WHAMM | RBL1 | TCN2 | CYP2E1 | DHX29 | ZNF594 |
| LEAP2 | AC019097.7 | ACIN1 | KIAA1324L | RP11-360F5.3 | HECTD3 |
| EXOC5 | B3GNT2 | RP5-1028K7.3 | RP11-178C3.2 | RPS2P55 | AMPD2 |
| RP11-540O11.1 | TSSC4 | HIST1H4L | SPC24 | SNX18 | CASP2 |
| C19orf57 | USP38 | AKR7A3 | CTBP1-AS2 | ZNF48 | MKKS |
| FSTL1 | ZBTB17 | TOP2B | CAT | HMGN1P38 | RBM12B |
| NDUFV2-AS1 | NUFIP1P | MUS81 | FAM13A | SDF2 | RP11-24M17.4 |
| MSH6 | AC007463.2 | RP11-115C21.2 | RP11-893F2.14 | HMGB1 | COL10A1 |
| RARB | AP001412.1 | POLR3B | PFKP | BCL7C | SDHAP2 |
| TCTEX1D2 | SOCS2P2 | RP11-392P7.1 | ACP2 | ARSA | AKAP12 |
| TMTC4 | SENP3 | CTC-429L19.3 | RP11-368J21.3 | RP11-325L7.1 | ANKRD17 |
| GS1-279B7.2 | PRPSAP1 | ZNF654 | AP5B1 | WDR83 | RP11-274A11.4 |
| ALS2 | C19orf44 | GPRASP2 | FAM204A | RNVU1-14 | TPM3P8 |
| RHPN1 | TLN1 | RP11-203J24.9 | RP11-646I6.6 | KDM3B | CEBPG |
| PGGT1B | GSTM2 | SNRPG | PLK3 | RP3-402G11.25 | C10orf71 |
| CLEC2D | SCML2P2 | RP11-110H1.4 | AC002059.10 | RP11-395N3.2 | SERINC2 |
| RP11-568J23.5 | JMJD6 | HOOK3 | SUPT5H | CCNK | RNF139 |
| UBBP4 | NEIL3 | CEP128 | ZBTB12 | RP11-248J18.2 | CCDC81 |
| RP11-421N8.1 | CNGA1 | CCDC138 | SNX10 | PITHD1 | ZBTB33 |
| RP11-176H8.1 | BTBD7 | RP11-1348G14.8 | RP11-264B17.3 | RP11-75L1.2 | AC096772.6 |
| PCAT14 | PMS2P10 | LINC00665 | AC011330.5 | H2AFV | RP11-435O5.7 |
| PTMS | RP11-62C3.8 | RP11-10L7.1 | LILRB3 | SLC22A20 | MSANTD4 |
| TADA2B | PSMD11 | PSMC1 | TMC3 | NPM1P25 | AL023875.1 |
| MTIF3 | RP11-75C10.9 | STOX2 | PRR19 | DPCD | HIST2H2AA3 |
| BIK | DOK1 | ATG4A | TMEM191B | RP11-526D8.11 | DNAJC25-GNG10 |
| ZKSCAN4 | KCNN4 | L1CAM | ACO1 | PRNP | AP001469.7 |
| OSER1 | PRR13P5 | PTPN18 | COPB1 | UBE2I | TVP23C |
| PIK3CD-AS1 | RABEP2 | TRNAU1AP | PPM1E | C9orf66 | MYH7B |
| ATG10 | KIF22 | DLEC1 | CDKN1B | IRGQ | RFPL3S |
| RP11-425D17.2 | LINC01023 | RP1-56K13.3 | SPEN | CNOT10 | NCK1 |
| HERC1 | ZNF609 | NKD1 | CDH12P3 | CDH12P1 | CTPS1 |
| CCDC74B | RP11-466A19.7 | MTHFD1P1 | RFFL | MTMR6 | RP13-941N14.1 |
| CTC-429P9.2 | PDZD11 | CENPBD1P1 | RP11-701H24.8 | RP11-498C9.12 | ZNF468 |
| RBMX | PLCE1 | RP11-429P3.5 | GPR83 | SOCS5 | CTD-2619J13.9 |
| RAB8B | SERHL2 | PRDM2 | CTB-55O6.8 | ZSCAN2 | LINC00265 |
| NUCKS1 | CROCC | PP2D1 | SP3 | ZSCAN16 | RP11-574F21.2 |
| TAF4 | NOTCH2 | LNX2 | RNPS1P1 | IL4I1 | DBF4B |
| XYLT2 | SMARCA4 | RP11-566E18.1 | RP11-83N9.5 | SPSB2 | PRKCH |
| CTD-2207O23.3 | RP11-276M12.1 | CTSB | TMEM18 | TXNL1 | PHBP9 |
| SYT12 | WNK2 | PIP5K1P1 | SERAC1 | MED19 | RP5-983L19.2 |
| R3HCC1 | UBXN2B | C5orf56 | ADNP | GS1-114I9.3 | GSK3A |
| RP11-33B1.1 | ARMC6 | CORO7 | ASB3 | WAS | DHX30 |
| ZNF781 | MRPS6 | RP11-90B9.3 | ZFYVE1 | RP11-656D10.3 | MAPK1 |
| PLEKHM2 | HNRNPA3P3 | DGCR8 | HIST1H4I | SERPINF2 | RP11-536K7.5 |
| ZMYM5 | IGLV9-49 | DVL2 | GLRX3 | RP11-542A14.1 | RP1-41P2.7 |
| SMG8 | FAM122C | RP11-701H24.4 | SP4 | RAB21 | MKRN2 |
| ZNF649 | TCF4 | COIL | RP11-439L18.1 | ZC3H13 | C8orf59 |
| CCDC174 | CTD-2323K18.1 | RP11-773H22.4 | C1DP1 | PALB2 | ZNF608 |
| TCTN3 | TPRN | PCDHGA9 | HSPA8 | RBM22P2 | TCEA2 |
| SNORA73A | SHOC2 | POLA2 | TPT1 | SRSF3 | AC004987.9 |
| AC010492.2 | ZNF767P | KIAA0391 | SMG7-AS1 | NPDC1 | RP11-401P9.5 |
| C4orf19 | C2orf44 | BLK | VPS37D | ANKRD13C | PAIP2 |
| MCM3AP-AS1 | AGO1 | DNAL1 | LPAL2 | SPIB | MDH1B |
| RNVU1-6 | RBBP4P1 | LIN9 | ADHFE1 | ASAH2B | RP11-361L15.5 |
| Clorf116 | PUS7L | KDELR3 | CYCSP34 | ULK4P3 | RP11-446E24.4 |
| CTD-3105H18.18 | PDE3B | RP11-572P18.1 | TBRG4 | PHACTR2P1 | RP11-275F13.3 |
| ZNF524 | RP3-337H4.8 | RP11-325L7.2 | RP11-275I4.2 | NFYA | EEF1A1P11 |
| UBE2W | CCDC66 | GDE1 | INPPL1 | ADAM20P1 | LINC00152 |
| RP11-255H23.2 | PTGES3P1 | RP11-977G19.11 | ZNF101P2 | SUPT20H | B3GNT8 |
| KRT18P4 | RP11-6L6.4 | RNF103-CHMP3 | CHD2 | ZC2HC1A | CPQ |
| ZFYVE21 | NUDT1 | LRRFIP2 | VPRBP | C17orf107 | AC018462.2 |
| RP11-111K18.1 | TTLL3 | CD226 | LRRC14 | DPH3 | GTF2F1 |
| ZNF281 | RP11-182J1.12 | SRRD | DGCR14 | PCAT6 | CYTIP |
| RALGAPA1P | OTX1 | SMC4 | TINCR | HTRA2 | SLTM |
| VLDLR-AS1 | PEX1 | KATNBL1 | CT45A11P | C10orf2 | AP1M1 |
| CTD-2666L21.1 | SMG1P2 | ARHGEF11 | NIPSNAP1 | C2orf76 | ZAP70 |
| BAAT | RP11-144L1.8 | C6orf226 | TNRC18 | ARL6IP4 | RP11-177H13.2 |
| FAM160B1 | ATG9A | ATP5E | RNVU1-4 | KSR2 | LLNLR-304A6.2 |
| GAPDH | RAB11A | CYBRD1 | ATF4 | RP11-213G2.3 | CTC-436K13.3 |
| ABHD5 | SGPL1 | LPCAT4 | SH3TC1 | ST13P6 | CTD-2382E5.6 |
| COX7CP1 | AMMECR1 | RP11-276E15.4 | UCN | RP1-86C11.7 | RP11-667F9.1 |
| SCNN1B | PAFAH2 | CTD-2547G23.4 | SSBP3-AS1 | ATIC | RP11-1391J7.1 |
| BRPF1 | CTD-2357A8.3 | KLHL5 | MYNN | NFYC-AS1 | INSR |
| SENP6 | LYPD3 | PPP1R12A | RP11-390F4.6 | MYBL1 | ARHGEF25 |
| CEP112 | RP11-667F14.1 | RP11-338K17.5 | CTD-2192J16.15 | RP11-304L19.11 | SCAF11 |
| BRD4 | FSIP1 | PAIP2B | RP11-135L13.4 | AC007952.5 | RP11-533E19.5 |
| AC006538.1 | AC002056.3 | RP11-11N9.4 | DOCK2 | SHFM1 | RNF214 |
| FAM229B | RPL35P5 | OSGEPL1 | ETS1 | AC094019.4 | TGS1 |
| PKM | IDH1-AS1 | NHEJ1 | RP11-108M9.3 | CAMSAP3 | RP11-147L13.8 |
| RNF185-AS1 | APC2 | RBM22 | KCNQ4 | FAM161B | HMGB3 |
| RP11-344B23.2 | PPP2R5C | RP11-520H14.7 | LRRC29 | RP13-465B17.4 | RP13-465B17.4 |
| HSP90AA5P | TTC34 | RP11-10A14.9 | ARMCX5 | OSGIN1 | ANKRD36B |
| RP11-755F10.3 | CTC-487M23.7 | AP000350.6 | DUSP1 | CCDC113 | ZBED6 |
| STKLD1 | OGFOD2 | GHET1 | COL7A1 | COX6C | RBM14 |
| UBAC1 | RP11-927P21.1 | TCL1B | LZTS2 | RP3-395M20.12 | TUG1 |
| HIST1H4J | RP11-297L17.6 | FAM170B | KLHDC3 | RP11-394I13.2 | INVS |
| ENAM | GSK3B | NCOA4 | AC009506.1 | FAM111A | HNRNPA1 |
| CHMP4BP1 | CDKL4 | RLTPR | AF131215.8 | TBC1D15 | RP11-147L13.14 |
| CFP | GDPD3 | EEF1A1P19 | KDM3A | NCLP1 | RP11-553D4.2 |
| RP11-390F4.3 | SOCS1 | PLCB2 | KCTD9 | ATP2B1 | RP11-463O9.1 |
| PCDHB9 | CDKAL1 | SMARCE1 | TTC25 | SV2A | RNF7 |
| TBCC | PRL | AC110615.1 | ALG12 | RPPH1 | AC092301.3 |
| ECD | RP11-629B11.5 | SLC39A8 | MS4A7 | CH17-258A22.4 | UTAT33 |
| IL6R | USP27X-AS1 | COMMD10 | RNF145 | WDR20 | CRNKL1 |
| MLLT6 | TMEM139 | SCFD2 | ZNF782 | ZNF786 | TP53RK |
| RP11-173M1.5 | STIP1 | LINC01420 | WNT5B | HMGN1P36 | CLASRP |
| MORF4L1P1 | CDK5RAP2 | BIRC2 | AC090286.2 | AC006116.22 | ZNF529 |
| ADGRG2 | PPP1R9B | RP11-297B17.2 | FUS | ATG5 | RP11-307L3.2 |
| CTB-161M19.4 | RP11-649A18.5 | NAMPT | TUBB8P2 | TRIB2 | RP11-961A15.1 |
| RP11-118H4.1 | TACC3 | RPL24P8 | RP11-849F2.7 | RP5-875O13.7 | NPTN |
| ANKRD10 | LIMS2 | TPX2 | RP4-545K15.5 | GTSF1L | CROT |
| RP11-153M3.1 | HMBOX1 | DIP2A | GOLGA6L9 | RUBCN | ZNF225 |
| GLYR1 | CTD-2047H16.4 | UBA2 | EXOC3L1 | MYH10 | HMGA1P2 |
| PSPC1 | PHF6 | RP11-528A10.2 | AC002550.6 | ITGB1BP1 | SEC16B |
| LINC00640 | HLA-B | SEC61G | DMTF1 | TSPAN10 | RP11-968A15.2 |
| DEXI | EHMT1-IT1 | NUTF2 | UBN1 | SNRNP200 | C19orf43 |
| AC006042.7 | STRN4 | FHDC1 | TPGS2 | RP11-359E3.4 | SLC4A9 |
| RNF122 | CLIC6 | ATF3 | RP4-665J23.4 | CSPG4P10 | NPIPB8 |
| RP11-350N15.5 | C5orf60 | TBC1D10A | KIF18A | TMEM231 | RN7SL192P |
| PDP1 | AC010091.1 | ID1 | CTA-313A17.2 | SLC39A6 | TRPC5 |
| PRMT2 | RP11-1023P17.2 | SPRN | TIGD5 | RP11-488I20.9 | ASAH2 |
| RNVU1-3 | RAB41 | ZNF417 | CTD-3131K8.2 | RPL12P12 | COPG2 |
| MANEA-AS1 | PRAP1 | ARMC5 | ACTR10 | C4orf46 | NOS1AP |
| TMEM189-UBE2V1 | RPS20P14 | NSMCE4A | NDUFA1 | CENPI | PCK1 |
| H1F0 | ZNF557 | GLYCTK | PPP3CB | KRT18P5 | NOP2 |
| RPS4X | RP13-415G19.2 | PDE1B | RNF8 | CYP1A2 | PKI55 |
| AE000661.37 | FBXL5 | AC073052.1 | PNISR | RP11-142C4.4 | DDX41 |
| RP1-221C16.8 | KB-1836B5.1 | TECTA | RP11-551G24.2 | RAC1P2 | PPP2CB |
| ALMS1 | RP5-1091N2.9 | RP11-493E12.3 | RP3-497J21.1 | RP11-651L5.3 | PECR |
| GPC5 | RIOK2 | TMEM56-RWDD3 | VPS8 | RRP7BP | PBLD |
| MAP3K11 | LYN | RP11-159G9.5 | ARG2 | CWC25 | IL7R |
| GLTSCR1L | RP11-894J14.5 | MRPL52 | LINC01176 | BCAR3 | TUT1 |
| BTG1 | IRF2 | CEP72 | B3GALT1 | ZNF559-ZNF177 | DSTYK |
| PRPF18 | FGG | NGRN | TRIOBP | IGLV2-8 | USP28 |
| RP11-463D19.2 | RP11-216M21.1 | SF3A3 | NUTM2B | CTD-2124B8.2 | AC005224.2 |
| AL589743.1 | C3orf38 | SUMO2 | CFAP58 | DYM | U2AF2 |
| ABCC11 | APBB3 | AC074391.1 | KANSL1L | RP5-1184F4.7 | GTF3C5 |
| CLASP2 | RP11-407G23.2 | LY75-CD302 | MBD3 | AGAP2 | RHOQP2 |
| AP001258.5 | KRBOX4 | SMN1 | RASSF7 | TEX21P | ST6GALNAC2 |
| RHBDD1 | YEATS4 | RPS24 | DAZAP1 | HPS5 | C22orf24 |
| RP11-108M9.5 | AC027612.1 | GSDMD | SERPINA10 | MAZ | RMND5A |
| RP11-492M23.2 | NRBP1 | DNAAFI | NUTM2G | RP1-286D6.5 | GUSBP2 |
| RP11-279O9.4 | HSPD1P1 | RPL21P110 | RARA-AS1 | HAGH | MAPKAPK5-AS1 |
| TRIM59 | IL11RA | GGA2 | EPHB6 | FSCN3 | ZNF346 |
| NR1D1 | RBM15B | ZPR1 | AC007279.2 | EBLN2 | ZDHHC16 |
| CSRNP1 | VPS18 | ZBTB11-AS1 | RPL12P1 | RP11-632K20.2 | RPL23AP64 |
| RP11-514O12.4 | SOCS6 | AC079767.4 | AC073333.8 | SUCLG2P2 | RP11-206L10.8 |
| BAD | TMSB4X | DOCK3 | C10orf12 | KIAA0141 | UBL3 |
| IGLV4-60 | KCTD10 | SUDS3 | RPS23 | RPL26P19 | RINL |
| SFPQ | E2F5 | ZNF566 | RP11-87G24.3 | ANK2 | SNX6 |
| RP11-541N10.3 | SRP72 | RP11-385F5.5 | RP11-536L3.4 | CTD-2541M15.3 | ELMSAN1 |
| ZBTB4 | MBTD1 | DCUN1D2 | IPO8 | CDK2AP1 | CMTM6 |
| HIST1H3F | RBBP9 | SPATA31B1P | SAR1B | RP11-108M9.6 | CDR2L |
| ZBTB24 | ARRDC1 | FKBP1B | AC079742.4 | TAF3 | FGD5-AS1 |
| CTD-3203P2.3 | RP11-155D18.13 | RP4-545L17.11 | KCNH8 | UHRF1BP1L | C1orf74 |
| HPN | TSTD2 | C9orf85 | ACAP1 | AC004383.3 | RP11-344N10.5 |
| S100A13 | SLC41A1 | ENSAP2 | EFCAB12 | DYRK1B | CNOT3 |
| POLR3D | CDC23 | DNAJC19P9 | RFTN1 | RHOT1P1 | PDZRN3 |
| RP11-297C4.6 | YKT6 | UBL4A | EPHX2 | CTC-338M12.6 | CH507-9B2.5 |
| RGPD1 | LRFN3 | RP11-140K17.3 | SF3B1 | LRRC75B | FBXO18 |
| SH3PXD2A | SLC4A5 | HMGXB4 | RP11-160E2.11 | TCAF1P1 | RP11-475O6.1 |
| RP11-572O17.1 | CHST12 | SPARCL1 | ZC3H3 | RP11-112J1.1 | UBE2E3 |
| PAPOLA | LINC00539 | RP11-313D6.3 | ZNF85 | WARS2 | CLDN11 |
| RP11-147L13.2 | STAM-AS1 | RP11-680H20.1 | EIF4HP2 | RP11-434H6.6 | AVEN |
| ZNF442 | AC000078.5 | RP4-675C20.4 | XKRX | PFN1P2 | BMS1P22 |
| EBAG9P1 | ZNF574 | KB-1980E6.2 | AC062028.1 | ULBP2 | RP11-25K21.1 |
| CFLAR-AS1 | RP1-283E3.8 | RP11-371A22.1 | RPL23AP42 | CHD5 | CTD-2319I12.4 |
| NAT14 | C1R | GABARAP | TPRA1 | COPZ1 | WHSC1 |
| RP11-810P12.7 | CTD-2054N24.2 | GLRX3P2 | CHD6 | ANP32A | TRIM52-AS1 |
| PAFAH1B1 | AC004069.2 | RP11-369G6.2 | DHX8 | OR7E7P | FZD7 |
| CCDC88B | RP4-814D15.2 | RP11-347C12.12 | SMAD1-AS2 | CTB-31N19.3 | CECR5 |
| HIST1H2BF | BRMS1L | CSF1 | C2orf27A | LMF2 | MGAT5 |
| C4A | HNRNPK | XXbac- | RP13-580F15.2 | ALDH4A1 | ROCK1P1 |
| | | BPG294E21.9 | | | |
| ADH5 | BRAF | RP11-542H15.1 | MAPKAPK5 | RP5-875H18.10 | KDM2A |
| CTD-2341M24.1 | WWC3 | MKL2 | MGEA5 | RP11-560J1.2 | PARP2 |
| RP11-727F15.9 | FKBP8 | C8orf76 | TIGD4 | DDB1 | KHDRBS1 |
| FAM9C | PCM1 | MBNL3 | UBA6-AS1 | LCP1 | SLC16A9 |
| USP1 | CTD-2027I19.2 | RPL13AP25 | PAN3-AS1 | CD38 | FOXD4L5 |
| CCDC144CP | C14orf93 | TEX10 | BOD1L1 | AC068831.10 | CTU1 |
| RP11-373N22.3 | CTD-3220F14.3 | SERTAD1 | LCORL | FTLP3 | MRPL1 |
| RBM19 | PDCL | AC024704.2 | C1orf95 | ZNF121 | RPL31 |
| PPP4R3A | PTPRF | FAM234B | ZNF207 | ZBED8 | RP11-104N10.1 |
| KPNA2 | TSTA3 | AP000442.1 | FKBP5 | KLHL42 | NUSAP1 |
| FLJ21408 | NUMA1 | RP4-756H11.3 | RP5-1092A11.5 | ZNF532 | RP11-426C22.4 |
| PHACTR1 | C16orf89 | GNRHR | CDK3 | AC147651.4 | IGHV4-34 |
| CAHM | RP11-85A1.3 | ADRM1 | RP11-133K1.11 | MCM2 | RAD54B |
| ZNF232 | CC2D1A | TMEM238 | TNFRSF25 | NONO | LINC00623 |
| CHCHD4P3 | AC073254.1 | TOP1 | SUPT6H | HSP90AA1 | DDTL |
| LINC00933 | BICD1 | FAM118B | RP11-96K19.2 | RPA1 | PPP1R35 |
| SLC28A2 | NUDCD1 | RP3-322G13.7 | CRTC3 | RBP5 | C9orf40 |
| CAPN15 | N4BP1 | ZBTB22 | ALG11 | MYO9B | CTD-3113P16.11 |
| POLR2C | SCARNA2 | SUMO2P1 | RP11-245J9.6 | YIPF5 | ENO4 |
| CTD-2231E14.2 | AC097713.4 | COQ5 | IFT43 | CSTF2 | RP11-144G6.12 |
| GSDMA | SLC25A43 | UQCC1 | PSEN1 | THEG | C1orfl32 |
| MORC2 | SKA1 | ZNF8 | CACNA1C-AS2 | MECR | RP11-173P15.9 |
| MMP25 | GPX4 | C2CD5 | TARS | RP11-98J23.2 | ZNF275 |
| GNB2 | CTB-47B11.3 | RP11-178L8.4 | EHBP1 | KIF1C | AC093838.4 |
| RPL5P18 | ZNF33B | TCF7L1 | PEX2 | RGS9 | GAB1 |
| ALCAM | RP11-366M4.11 | WDR44 | DTL | ZNF565 | ATF4P3 |
| LA16c-360H6.1 | SCYL2 | MOK | RP11-157P1.4 | ERMARD | RPL35AP |
| PRRC2A | MIOS | UBE2SP1 | SELV | MPRIP-AS1 | RP13-16H11.5 |
| AD000864.6 | GATSL3 | GPSM2 | CCDC97 | CTC-425F1.4 | CRP |
| CDK8 | CPSF6 | RP1-37M3.8 | IGLVI-70 | RP4-583P15.14 | MMS19 |
| SESN3 | AC006509.7 | RP11-562A8.4 | HSPA8P7 | ERBB4 | TUBB2B |
| MKLN1 | GBE1 | NUB1 | RP5-1125A11.7 | RSPH14 | PPP1R1B |
| ARHGEF18 | CHAD | RP11-160H22.5 | ZSCAN32 | RP11-379H18.1 | RP11-285J16.1 |
| CDC25C | RAB39A | AP000476.1 | LINC01372 | FAM21A | NDC80 |
| TP53TG1 | CACNA2D2 | CISTR | ADRB2 | CLSPN | RP11-428J1.4 |
| MMP7 | MGMT | PDK1 | UBA6 | TMC4 | NUDT5 |
| ZNF778 | SLC37A2 | PARL | TRAPPC1 | RP11-87H9.5 | ITGAL |
| BEND3P1 | XXbac- | SAA1 | RP11-464F9.22 | ORC6 | CAPRIN2 |
| | B476C20.9 | | | | |
| AC007256.5 | PRMT7 | CHAF1A | FAM71D | MGAT1 | ZNF233 |
| GMFG | IGLV3-19 | UTP18 | SMNDC1 | FOXD4L3 | TMEM123 |
| PIGY | WDR89 | RAP2C-AS1 | PITRM1-AS1 | APOE | CLTC |
| IGKV1-12 | SPECC1 | CH17-437K3.1 | RRM2 | MIATNB | HIST1H2AK |
| ANKRD11 | RP11-466H18.1 | TTC37 | SEMA4D | KCTD21 | ORMDL2 |
| CEMIP | GPR39 | PLEKHM1P | CTC-471F3.5 | IRX5 | TMPO |
| ZNF345 | KCTD18 | CAMK2D | PRKCQ | TMEM86A | SNORA63 |
| RP11-96L14.7 | TRIM39-RPP21 | NDUFA4 | LINC01251 | KLRC4-KLRK1 | BTN3A3 |
| SEL1L3 | NUDT3 | IGLC1 | USF2 | HEATR5B | SAE1 |
| CTD-3138B18.6 | MIER3 | GPATCH11 | OCIAD2 | CTD-2192J16.22 | AC104134.2 |
| RP4-620E11.8 | CTCF | RP1-43E13.2 | RP11-57K17.1 | CCDC80 | RP11-21L23.2 |
| C5orf28 | BCCIP | ZNF280C | GAA | RP11-119F7.5 | EFCAB5 |
| RPL24P4 | CIC | SLC38A2 | RP1-168L15.6 | MMACHC | C8B |
| PIFO | ERAP1 | GAN | FHL3 | ZNF672 | AC009060.3 |
| LRRC40 | PLPPR2 | FCHSD2 | GOLGA8IP | RPP38 | TBC1D9B |
| KIAA0196 | HIST1H2AJ | DNAJB1 | SPATA3-AS1 | IGKV2D-30 | SFTPB |
| CDCA8 | RP11-334E6.3 | HMGB1P5 | ABHD17B | AC142472.6 | MTND4P15 |
| CMBL | RP11-1105G2.3 | LMBR1L | CIZ1 | ALDH1L2 | POMGNT1 |
| AC138969.4 | ALOX5AP | ONECUT1 | ARID1A | BMPR1APS1 | HS1BP3 |
| PRKY | RP11-385F5.4 | SMIM5 | TTLL11-IT1 | FANCD2P2 | ZNF3 |
| FABP1 | PPP1R12B | DHODH | ATP10B | TRIP4 | GIPC1 |
| RP4-761J14.10 | THUMPD2 | CCAR1 | LRRC42 | TBC1D10B | MEF2C |
| RP11-61A14.4 | RP11-331F9.3 | PLCD4 | CPM | SMARCC1 | KIF23 |
| NIN | PHKB | NBPF25P | RNF139-AS1 | GUSBP4 | IGKV3-11 |
| MIR497HG | RP11-332H18.3 | AC068831.6 | CTD-3252C9.4 | HSPA5 | NCAPD2 |
| COMMD7 | AC004471.9 | CDC26 | ING3 | APC | SPTB |
| SERPINB6 | ZNF449 | RP11-94I2.4 | SETD5 | HOXA3 | SSRP1 |
| RPL7AP10 | BBS10 | RLF | KLHDC4 | RP5-1073O3.2 | KIAA1524 |
| AC005682.5 | AAED1 | MED23 | CTD-2649C14.2 | RP11-326I11.4 | C17orf89 |
| CROCCP4 | RP11-289I10.2 | DSCR9 | IK | EIF1B | RP11-22B23.1 |
| RASSF1-AS1 | DDIT4L | TARDBP | TMEM108 | HNRNPM | TTC7A |
| ZDHHC17 | XXbac- | RP11-290F24.6 | YIF1A | AMN1 | BUD13 |
| | B444P24.13 | | | | |
| DDC | LSP1 | AC006994.1 | SBSPON | SHKBP1 | KB-1592A4.15 |
| RP11-84C10.4 | RP11-329A14.1 | CTA-246H3.11 | AOX2P | EIF3FP3 | ZSWIM3 |
| RP11-46J23.1 | CTC-524C5.2 | LDAH | MYO1H | RP11-644F5.10 | GPR108 |
| YTHDF3 | UBB | WTAP | NPM1P37 | RAD54L2 | TRUB1 |
| RP11-662J14.2 | HNRNPC | CDS2 | ANAPC7 | RP11-545M17.1 | ZNF706 |
| SFXN3 | CLECL1 | RP11-264B17.4 | RP4-753P9.3 | ARL6 | HIF1A-AS1 |
| CD79A | GPR146 | AC138035.2 | RP11-108O10.8 | RNU1-28P | RNU1-27P |
| RNU1-1 | RNVU1-18 | RNU1-2 | RNU1-4 | RNU1-3 | U1 |
| U1 | SIAH1 | SCNN1D | RP11-798M19.3 | FHL1 | RP11-313J2.1 |
| RAB8A | SYK | PUS3 | LRR1 | KIAA0232 | RBM39 |
| RP11-396C23.2 | SMG9 | PAQR7 | RPSAP54 | RP11-113D19.9 | CTD-2619J13.14 |
| SCARNA5 | CCDC127 | RP11-534L6.3 | DNALI1 | RPS20 | ZMYM1 |
| ANKRD46 | GALNT7 | PDE8A | TMEM64 | YTHDF2 | RAB5B |
| RP11-615I2.6 | PARG | RP11-174O3.1 | KRT10 | UBQLN1 | IGLV1-40 |
| POLL | RP11-35J23.1 | RP11-215A21.2 | RP11-126K1.8 | MOSPD1 | AC002117.1 |
| SCRN2 | CTD-2260A17.1 | NEXN | RP11-252C15.1 | RNF40 | CTC-499B15.5 |
| G6PC | TP53 | GCC1 | MAP3K9 | ZNF517 | RP11-298I3.3 |
| PTMAP2 | PHF3 | CCDC9 | SLC25A1P5 | GLUD2 | RP11-152H18.3 |
| CHGB | CLINT1 | ATP5G2 | TIGD7 | AC025335.1 | KB-1208A12.3 |
| RP11-297C4.1 | RP11-305O6.4 | RNF38 | UNC5CL | TERF2 | KANK3 |
| HNRNPA2B1 | AC078899.1 | RP11-517B11.7 | MARK2 | RP11-44N11.2 | RP1-118J21.25 |
| RP1-63M2.7 | RP11-785D18.3 | RNF103-CHMP3 | CMSS1 | RNU2-63P | ARL15 |
| HEATR6 | CTD-2021A8.3 | MAFA | GPR174 | CTD-2270P14.2 | HYLS1 |
| RER1 | RP11-252A24.2 | SLX1B | MBIP | FIBP | TOLLIP-AS1 |
| ZNF43 | ASNA1 | ZNF419 | LRRK2 | C5orf34 | TATDN1P1 |
| PODNL1 | CTD-2006C1.2 | RP11-286N22.8 | CTD-2540L5.5 | SELK | SIM2 |
| AC073046.25 | RP11-816J6.3 | DDX3Y | FAM46D | TEF | TINF2 |
| FSIP2 | RP11-717D12.1 | SEC31B | PCDH11X | RP1-159M24.1 | SCARNA16 |
| C9orf147 | KIAA0922 | PTK2B | RP11-552F3.9 | DCLRE1B | PHF1 |
| UBE4B | RP11-730G20.2 | RP11-253A20.1 | ENTPD1-AS1 | RP3-508I15.22 | RP11-386M24.4 |
| TAX1BP1 | RP11-304L19.13 | OPRL1 | C19orf68 | DBI | WASH4P |
| AC022210.2 | RP11-361L15.3 | SPC25 | ZEB1 | ARL5B | POLR2J4 |
| YBX3 | PLXNA3 | RP11-805J14.5 | LINC01003 | CTD-3199J23.6 | CTD-2192J16.26 |
| AC007405.6 | C15orf38-AP3S2 | PHEX | RP11-365F18.6 | C10orf95 | NFE2L2 |
| ERI2 | SNRPFP4 | NPM1P39 | RP11-343H5.6 | BTBD10P2 | ASCC3 |
| IP6K2 | KCNN1 | MLKL | ACYP2 | RP4-784A16.2 | CPSF7 |
| RP4-539M6.22 | PCSK5 | ARRB1 | TRAF2 | CCP110 | EEPD1 |
| VCP | NME1-NME2 | RAB28 | AC020743.3 | PLS3 | NOTCH3 |
| EPC1 | PCCA | CNOT6 | IGFBP4 | PCBP4 | RP13-942N8.1 |
| SCRG1 | GMIP | NUP155 | RHOG | VDAC1P2 | RP4-724E16.2 |
| GNB1 | YWHAQ | SLC16A7 | LINC01183 | H2AFY2 | RP11-480G7.2 |
| RPS19BP1 | NUP153 | TNFRSF10A | CAPZA2 | POM121C | LINC00310 |
| ZNF211 | RP11-724O16.1 | SVIL-AS1 | ZNF273 | RP11-173A16.2 | RP11-712L6.7 |
| RP11-170K4.2 | RP4-583P15.10 | RAD9B | PCLO | RP11-455F5.6 | C2orf49 |
| CTD-2524L6.3 | RP4-622L5.7 | PBX1 | AP001469.5 | AC083884.8 | CERS1 |
| LINC00954 | LRRK1 | ZNF569 | RP11-245C17.2 | DHX40P1 | RP11-455I9.1 |
| RUFY1 | DSCC1 | ARL6IP5 | RP11-459F6.3 | IGHEP2 | ZPBP2 |
| WDR62 | RFX3 | EIF4A3 | PRMT9 | AC079448.1 | MAT2B |
| RP11-285G1.14 | MTCYBP18 | LL0XNC01-240C2.1 | RP11-68I3.10 | INTS12 | SLC7A5P1 |
| TOE1 | RP11-574F21.3 | ADPRM | AC007566.10 | FNBP4 | CDCA7L |
| RP11-495K9.5 | CHD7 | NNMT | RBM10 | CTC-277H1.6 | FAM98B |
| DNTTIP2 | ZMYM4 | RP11-843P14.1 | AC007906.1 | FAM168B | RP4-756H11.5 |
| SLC4A1AP | RP11-108M9.4 | ZNF607 | TESPA1 | RPL21 | G6PD |
| RP11-164H13.1 | PRAME | ARHGAP6 | KIF3C | AC006129.1 | AFF2 |
| NHLH1 | SAYSD1 | FNTAL1 | KIF14 | BMF | RP11-3P17.5 |
| TRMT12 | AC006116.13 | DDX20 | RP4-803J11.2 | SPPL3 | ZNF408 |
| RP11-147I3.1 | ZNF776 | CTB-50L17.10 | RP11-22OI1.2 | ARRDC3 | CTC-529I10.1 |
| RPL39L | EIF3EP1 | ZHX2 | AMER1 | RPL26 | RP11-353N4.6 |
| MDH1 | LINC00526 | ATXN1L | ZEB1-AS1 | MINOS1-NBL1 | TTLL9 |
| ZNF143 | RNF219 | TSC2 | AC005775.2 | ZNF337 | NINL |
| AMBN | CCDC7 | CTD-2544H17.1 | RECQL | APOLD1 | RP11-532F6.5 |
| SALL2 | ACTR5 | CAND1 | AP005901.1 | SPSB3 | RP11-305M3.2 |
| GOLGA3 | RAD23B | SLC25A47P1 | YWHAZP4 | PSMD14 | C1QL4 |
| ESPL1 | SNX30 | NBEAP3 | RP11-463O12.5 | RP11-35G22.1 | RP5-1042K10.14 |
| NDUFA9 | MXRA7 | RP11-10L12.2 | RP1-283E3.4 | XXbac- | ABCC1 |
| | | | | BPGBPG55C20.1 | |
| WIPI1 | CRIM1 | FRG1BP | RP11-173E2.1 | ZNF431 | CCDC157 |
| CPNE1 | HUS1B | CTB-4E7.1 | ALAS1 | DIAPH1 | PBX2P1 |
| RPL10AP6 | AGPAT1 | ORMDL3 | CDC5L | NPM1P29 | SPATA9 |
| RP11-490H24.5 | RP11-347C18.1 | RP5-1142A6.9 | RMRP | TNKS1BP1 | CTD-2313J17.1 |
| RP11-10N16.2 | MSNP1 | RWDD4 | EPHX3 | ZNF385C | RP11-368N21.5 |
| RPS23P8 | MED1 | C11orf68 | RP11-177C12.4 | CTD-3075F15.1 | RP11-488C13.7 |
| SLC35B1 | HLCS | RN7SL832P | RP11-613F22.6 | EID1 | RP11-295H24.3 |
| RP11-231G3.1 | AMIGO1 | CPSF3L | REPS1 | RP11-138I1.2 | QSOX2 |
| SMG6 | MAP2K7 | AC015849.13 | IER2 | SYNGR1 | KLC4 |
| SFMBT1 | RP11-256L11.3 | WTIP | TAS2R50 | RPS29 | RP13-516M14.2 |
| AC097495.3 | CTD-3138B18.5 | ANO1 | C17orf100 | TRAIP | ABALON |
| SRPK3 | EIF1 | PDIA4 | PFDN4 | SPIN4 | TNPO2 |
| ALDH1A3 | RP11-449P15.1 | SH3D21 | KATNBL1P6 | LGALS3 | SCARNA7 |
| USP51 | RP11-552C15.1 | LASP1 | XRCC2 | SORBS1 | CCDC15 |
| MED9 | PDE1A | AP000866.1 | KB-1125A3.11 | ZNF865 | ACTL10 |
| NCAPH | RP11-68I3.4 | MORF4L2 | CTD-2184D3.7 | MZT2B | MMP19 |
| RP11-370B6.1 | HYAL1 | PTGES3P3 | AP000577.2 | ASCC2 | Sep-05 |
| LINC01346 | TMEM191C | SPATA20 | RP3-512E2.2 | EFNB3 | AC108479.2 |
| PRDM10 | TRAPPC12-AS1 | RP11-69L16.5 | ZUFSP | RAB33B | BAK1P1 |
| GPA33 | MMRN2 | TXLNA | CUL4B | RP11-715J22.3 | ZNF32-AS1 |
| FAM134A | RPL34 | ZFP64 | CSNK2A1 | IFITM4P | RP11-259G18.1 |
| FAM180B | RP11-173B14.4 | EXO1 | RP11-158M2.3 | MLEC | AATBC |
| RP11-862L9.2 | TBC1D13 | ZFAT | USP9Y | FLJ13224 | PCDHGC5 |
| AC018766.4 | RP11-486G15.2 | SLC25A18 | IPO5 | RAB6C-AS1 | QPCT |
| RP11-414C23.1 | RP11-288K12.1 | NDUFA5P11 | COX7C | CSTA | CYP2D7 |
| CTD-2369P2.5 | TMEM200A | MGAT3 | ZNF184 | RP11-755B10.3 | AC012368.1 |
| EXOSC10 | HPCAL4 | RP11-347C18.3 | FLVCR2 | RP11-546K22.1 | NFKBIA |
| RP11-679B19.1 | AC017074.2 | FRRS1 | RP11-482D24.3 | GJA9 | PHAX |
| CXXC1 | RXRA | SUZ12P1 | ZNF627 | CPSF2 | LINC00657 |
| TTBK1 | PLEKHA3P1 | LRRC37A | RP3-375P9.2 | SLX1A | HUWE1 |
| RP11-70L8.5 | CBX3 | PLCB1 | ABHD3 | RP5-1057120.6 | RP11-455G16.1 |
| GTSE1 | FPGT | RAB11FIP5 | RP11-131M11.2 | FOXO4 | RP11-1137G4.4 |
| CSDC2 | ZBTB8OSP2 | RP11-548H18.2 | EIF4G2 | DYNLRB1 | BBS12 |
| TVP23C-CDRT4 | RP11-572M11.3 | ARID5A | MEGF8 | GLYATL1P4 | NFKBID |
| CTD-2509G16.3 | RP5-1024G6.5 | RP11-12D24.10 | EVI5 | RP11-550I24.2 | IGHMBP2 |
| APOA2 | CPEB3 | RP1-20N2.8 | RUNDC1 | HS3ST3B1 | THOC5 |
| DCLRE1A | RP5-875O13.6 | AC137932.4 | GNAS | CTD-2035E11.3 | METTL21A |
| SMC2-AS1 | PPP1R18 | SLC39A13 | FAHD1 | PROM2 | RPL10 |
| PLEKHG5 | AC018755.17 | AMD1P3 | CYP26A1 | CCDC107 | AC007325.1 |
| ATAD3A | RP5-827C21.6 | TTC28-AS1_2 | SNX2 | PCBP2-OT1 | HSD17B1 |
| CCDC71L | MAP4K3 | STK16 | CSPG5 | CNOT8 | NDUFS5 |
| AP004290.1 | FGB | PARGP1 | DEPDC4 | RP11-981G7.6 | CDKL3 |
| ZPBP | RP11-923I11.3 | NADK | CENPJ | RPL7AP60 | RP11-325L12.5 |
| IGKV2D-26 | CFAP45 | NPIPB5 | AC003090.1 | GUCA1A | TCOF1 |
| SH3KBP1 | CDKN3 | ZNF846 | LL21NC02-1C16.2 | RPLP1P11 | CCL3 |
| ZNF571-AS1 | ALDH5A1 | SMAD7 | RP11-950C14.7 | RP11-347C12.4 | RP11-345J4.4 |
| DBN1 | GS1-590J6.3 | RP11-209D14.4 | KLF3 | HNRNPDL | CYP2A6 |
| LONP2 | MTF2 | MTCO3P11 | PMS2P2 | CPT1C | ATF7 |
| TRIM34 | GTF3C3 | TTC39B | RP11-752D24.2 | RP11-468E2.1 | KDM4A |
| KAT2B | RP11-44N11.1 | PAPOLG | PSMA3-AS1 | ITGB2-AS1 | ANAPC1 |
| SHISA9 | AC090498.1 | TTC22 | OVGP1 | BICD2 | RP4-569M23.5 |
| AC009120.3 | IGHM | DROSHA | STK31 | HELLS | CASQ1 |
| RPS3AP2 | DNAJC13 | LDHAP7 | ZNF543 | PRDX3 | TTC16 |
| FAM20A | RP11-57A19.2 | TWISTNB | SLC38A1 | WAC-AS1 | FTH1P11 |
| MSMP | HNRNPD | AC004556.1 | RP1-20B11.2 | AC073842.19 | TMEM204 |
| PRELID3A | RP11-388C12.8 | SULT1A3 | RP11-287F9.2 | IFFO1 | AC016734.2 |
| ZNF436-AS1 | SPON1 | RPL10P15 | KRT18P28 | P3H1 | RP11-548H3.1 |
| MARK3 | CTD-2246P4.1 | AC090587.4 | HADH | STK26 | ARX |
| ENPP5 | DCAF5 | LRSAM1 | NPIPP1 | CCT2 | AURKC |
| SEC14L5 | PSMD9 | SIRT4 | CDC42 | FAM218A | ELK1 |
| RP11-49O14.2 | RPL6P27 | SRF | RP11-435O5.2 | RP11-847H18.3 | CTD-2591A1.1 |
| KLRK1 | RP11-777F6.3 | HSPE1 | SUMO3 | HOXA1 | MDM1 |
| NSG1 | SMIM10L1 | FGA | HEIH | ALDOB | ZKSCAN1 |
| AF129408.17 | CHRNE | Metazoa_SRP | RPS6KL1 | HCP5 | UVSSA |
| HSPE1-MOB4 | LRRTM2 | FDPSP7 | RP11-440L14.4 | RP11-314A20.1 | FGF13 |
| FBXW7 | TFAP2E | RP11-671J11.5 | NARFL | MTMR1 | F8A1 |
| RP11-531F16.4 | USP7 | AC093074.1 | DNAH10 | UBE2F | LINC01374 |
| GAB2 | ARID2 | SPNS3 | KCNK13 | EIF1AY | CDCA7 |
| TSKU | AC005606.14 | CDK9 | PHF23 | PPP5C | BTNL3 |
| RP11-282K24.3 | RP11-134D3.2 | CTA-223H9.9 | DCUN1D4 | HSD17B4 | EPM2A |
| NDUFA13 | AC000123.3 | RP11-482M8.1 | RP11-192H23.5 | FTH1P3 | ETV2 |
| RCBTB1 | SEC13P1 | AE000662.92 | LIMS3L | EIF4A1P7 | CDC6 |
| CAMK1 | RP11-464D20.2 | ZNF385B | RN7SL15P | KDM6A | SOS2 |
| RP11-730K11.1 | RP11-152N13.5 | SNRPGP15 | TWF1 | RP5-956O18.2 | TUG1_1 |
| SAP130 | LINC01119 | DEPDC7 | AC022007.5 | CTC-260E6.2 | RPL13AP5 |
| HMGN2 | TRIP12 | RP1-39G22.7 | GRIPAP1 | NXF1 | PHLPP2 |
| C18orf25 | FAF1 | RPL12 | RP13-753N3.1 | RP11-48G14.3 | VPS50 |
| UPK3BL | TADA3 | C1orf174 | EID2 | RP11-195L15.2 | C11orf58 |
| TRNP1 | RWDD3 | RP11-350J20.5 | NDUFA12 | CTD-3220F14.1 | DYNC1I2 |
| ZNF519 | TNPO1P1 | RN7SL381P | AC093166.1 | RPS11 | NUDT2 |
| SNRPD3 | PPA2 | CLPTM1 | CTD-2192J16.20 | CFL1P5 | ATG16L1 |
| ZMYND19 | ZNF236 | SMC2 | CYP3A7 | ENHO | RPL39P3 |
| ATXN7L3B | CAPN3 | BUB1 | RP1-257A7.4 | CLIC4P1 | KDM5C |
| FAM83D | UBE2G1 | GRTP1 | GIT2 | TCL1A | CTD-2235C13.1 |
| SRPRB | GNB3 | AP1G1 | FEM1B | U2AF1L5 | RP11-54O7.1 |
| DYNC1LI2 | KLK1 | SYT7 | C12orf29 | SRA1 | RP5-935K16.2 |
| SRBD1 | AC079807.2 | DBNL | C1orf54 | RP11-583F2.2 | DNAJC16 |
| RP11-571I18.5 | FRMD8 | RAN | COPZ2 | INO80B-WBP1 | POC5 |
| IRF3 | RP11-589P10.5 | RP11-420A23.1 | LRRC46 | CTD-2555A7.1 | RABL3 |
| HNRNPA3P5 | RIMBP3B | C6orf62 | RP11-95D17.1 | AZIN1-AS1 | RP11-346C20.3 |
| SPAG16 | RP11-460I13.6 | PAIP1P1 | CRABP2 | RP11-368P15.3 | TOPBP1 |
| SNX18P12 | TMEM183A | RP11-662J14.1 | USP14 | SPATA4 | CTC-493L21.1 |
| SLC25A39 | HNRNPA3P6 | RP11-839D17.3 | STUB1 | HMX2 | TRERF1 |
| XRCC3 | RP5-836N17.4 | ABHD13 | TRERNA1 | RP11-181C3.1 | UBE2Q2 |
| MEIS2 | RBM42 | C11orf80 | SYCE3 | RP11-64K12.4 | MALT1 |
| IGKV2-29 | C6orf132 | GCLC | GDI2P2 | RP11-408P14.1 | RIMKLBP2 |
| RP11-1157N2_B.2 | ZFYVE16 | FLJ31104 | CASP8AP2 | GPC6 | LINC00304 |
| GNE | FTOP1 | FGD4 | BBS2 | RP11-669E14.6 | RP13-33H18.1 |
| PPM1J | FXR1 | RP11-520A21.1 | AC004837.5 | ATE1 | UBL5 |
| NAALADL1 | STRADB | CDK13 | PTPN2P2 | CSNK2A3 | STIL |
| CTC-490E21.10 | S100A14 | NRIP1 | RP11-640N20.4 | ST6GALNAC4 | ADH1A |
| NMT2 | CRYZ | CTD-2006H14.2 | TSPAN6 | CTC-296K1.3 | KCNJ11 |
| TRPM4 | RP11-367J11.2 | EXO5 | TOPORS | ATP8B3 | GGT5 |
| RP11-659E9.4 | RPL8 | RAE1 | CTA-280A3.2 | RP11-706P11.2 | RAB6C |
| P2RX7 | PNMA6A | CLMP | RP11-793H13.10 | AC005780.1 | RPL3 |
| COX10 | FAM27B | GTF2IRD2B | HSD17B10 | SC22CB-1E7.1 | TUBA3FP |
| ZFPM1 | TUBB8P12 | KNTC1 | RP11-240G22.4 | BRF2 | ENTPD7 |
| RCN3 | FAM179B | H2BFS | STAU2 | RP11-247A12.2 | MESP1 |
| KDM8 | RP11-1193F23.1 | POLDIP3 | PI16 | PEX11G | CNOT7 |
| SERPINB9 | AL133243.1 | RP1-315G1.3 | RP11-434D9.1 | CALM2 | ANXA4 |
| METTL10 | RP11-889L3.1 | SNHG7 | RFC4 | RPS15A | EFCAB7 |
| VPS53 | CCDC88A | PTTG1 | BDH2P1 | RP11-365F18.3 | CICP22 |
| RP11-390E23.6 | NGFRAP1 | COX6A1 | SCG5 | CFAP53 | GAPDHP38 |
| SNAPC2 | RP11-148O21.2 | RP11-501C14.5 | RTEL1 | TACR2 | RP11-961A15.3 |
| PPP2R3A | ANKRD65 | RPS9 | H2AFZ | SLC26A4 | NT5CP1 |
| PLCD1 | RPIA | PSMD10P1 | SUCLG2-AS1 | SPAG5 | ARFIP1 |
| CTC-448F2.6 | RP11-257K9.8 | SLF2 | HNRNPH2 | RP11-494O16.4 | SLBP |
| MCTS2P | MIA | EEF1B2P3 | RP11-824M15.3 | RP11-74D7.3 | RP11-175D17.3 |
| GTF2IP1 | TMEM221 | PIGH | CCDC14 | CTB-33O18.3 | RAD51L3-RFFL |
| RP11-214N9.1 | FTH1P7 | BAZ1B | RP11-758H9.2 | VRK1 | ZNF784 |
| EIF4EP1 | RAB2B | CRY2 | ZNF506 | RP11-391L3.5 | SPINK13 |
| QRSL1P3 | RASGRP2 | SEL1L | ARHGAP32 | CTD-2659N19.9 | RP11-977G19.12 |
| RP11-326K13.4 | FGFBP3 | RP11-474P2.4 | ZDHHC11B | GINS2 | HSPE1P3 |
| CTD-3037G24.3 | PRPF6 | ZMYND12 | XXbac- | RP3-394A18.1 | RFC3 |
| | | | B135H6.18 | | |
| AC008592.3 | NDUFA9P1 | AC007246.3 | RP11-758M2.1 | RP11-656D10.5 | NDUFS6 |
| NFIL3 | RBMXL1 | CCDC28A | ZNF630 | IGHV3-72 | RP11-461L13.3 |
| RPS3AP5 | APCS | ACTR3B | PAM | METAP1 | SMPD3 |
| RP11-571I18.4 | RP11-88I18.2 | CTD-2370N5.3 | USP32 | SDHAF1 | RPS15AP16 |
| VEZT | CHST10 | CAGE1 | AC012513.4 | RP11-326C3.16 | MFSD3 |
| URM1 | CYP39A1 | RP11-204C16.4 | MAP4K1 | PKP3 | CLEC4A |
| IFI6 | TMEM159 | CIDEB | RBMS1P1 | RP5-943J3.2 | MCM6 |
| SOX2-OT | GPR87 | FRMPD1 | MED21 | GTF3C6 | ABCC9 |
| CTD-3105H18.8 | RPL10AP2 | FOXD2-AS1 | TLR4 | GPN1 | RTCA-AS1 |
| AC115617.2 | ZSCAN21 | RP11-110J1.2 | RPS18P9 | DHX40 | AC109829.1 |
| TRIM6 | FXYD3 | CCNB2 | AC016739.2 | HACL1 | GPR65 |
| GRWD1 | TTLL11 | HNRNPL | CHAMP1 | RP3-437C15.1 | BHLHB9 |
| NEURL4 | CDC42P4 | TRAF6 | UBD | CHL1-AS1 | ING1 |
| C12orf65 | LINC01554 | CTD-2145A24.4 | ZBTB40 | RAB3GAP2 | RP11-457M11.5 |
| HM13-AS1 | CDCA3 | PHBP6 | DIAPH3 | AUNIP | RP11-122K13.12 |
| SPATA5L1 | TATDN1 | SGOL1 | NPLOC4 | ARHGAP18 | U1 |
| ROGDI | ARPC3P1 | BACH1 | NOS3 | RP3-388N13.5 | KIF2C |
| ARL14EP | RP11-318C24.2 | TTR | RP5-864K19.4 | CTD-2235C13.3 | ZNF212 |
| RP11-665E10.5 | RPUSD1 | CTD-2554C21.1 | RP11-629N8.5 | TERF2IP | CD109 |
| NR2C1 | SCLT1 | MAX | TMF1 | TEX22 | PEBP1P3 |
| MYH15 | PIP5K1C | CTD-2325M2.1 | AP003391.1 | SET | RP5-1047A19.6 |
| MAN2A2 | RP11-575L7.4 | YBX1P10 | BABAM1 | RSRC2 | COL1A1 |
| FKTN | RP11-566K19.5 | BZW2 | DHDH | CLUHP3 | MGC16275 |
| RP11-261P13.6 | TNFAIP1 | TEP1 | DDX11 | PKD1L2 | FAM35DP |
| FBXL19 | JOSD2 | RPS18P12 | CTD-3099C6.11 | C17orf80 | HEXDC-IT1 |
| VPS51 | AC144652.1 | U2AF1L4 | MAPRE3 | RP13-270P17.2 | ZNF436 |
| CSTF1 | RP11-351C21.2 | RAB3GAP1 | CTD-2583P5.3 | CARS-AS1 | RP11-700P18.2 |
| NEDD8 | SETD6 | ACADVL | ATP2C2 | FAM185A | CDC14B |
| SPECC1L | RP11-345J4.6 | CTD-2619J13.8 | CWC22 | SMU1 | TMEM5 |
| SCP2 | EIF3D | ERF | XRCC6 | RP4-536B24.3 | SNF8 |
| CCDC90B | SAP30L | RP13-20L14.4 | GLYATL1P3 | DNAJC14 | RP11-517I3.2 |
| CTD-2537I9.12 | KAT7 | SPAG7 | RP11-203F10.6 | BNIP3P5 | C2orf81 |
| TUBB4B | RP11-795F19.5 | BSDC1 | RP11-196B3.3 | CCNA2 | CTD-2619J13.13 |
| SNX12 | FTH1P16 | RP11-817O13.9 | AARS2 | E2F3 | CD2AP |
| RP1-234P15.4 | SDK2 | GUSBP11 | ARL6IP1 | PPIAP16 | RSF1-IT2 |
| RNF187 | HTT | CDC27 | RASD2 | FOXO3 | HPRT1 |
| MAPK7 | RP11-727A23.10 | ADCY1 | RP5-1056L3.3 | ANAPC4 | LINC01266 |
| NRAV | TMEM19 | AC009950.1 | GLMN | SNX6P1 | NAA60 |
| EIF3L | RP11-317N8.3 | NDRG1 | DNAJB7 | RP11-311P8.2 | RP13-582O9.6 |
| PSTPIP2 | AQR | RP11-12A20.7 | C12orf4 | NRD1 | NRBF2 |
| CTD-2215E18.1 | RPS3AP47 | RP11-87C12.5 | CNFN | IQGAP2 | ZNF652 |
| AC015987.1 | JAG1 | RP11-251G23.2 | RP11-98D18.15 | TAS2R4 | SFSWAP |
| STX17 | SNORD3B-1 | HHIP | LTA4H | CCT7 | RPS19P3 |
| RP11-315I20.1 | STX8 | APOB | GLIPR1L2 | MIS18A | AC007308.7 |
| SNX9 | GSKIP | RP11-219B4.3 | CTSV | ARMC8 | AP1AR |
| AC015971.2 | IFNGR2 | NAT16 | CHCHD2P6 | PNRC2P1 | ZCCHC6 |
| FAM35BP | RP13-93L13.2 | CTD-3220F14.2 | CTD-2509G16.5 | RGS17 | SCAF8 |
| REPS2 | KLHDC2 | RP11-435J9.2 | CCNI | KCNT1 | CTDP1 |
| DVL3 | CHMP4B | RP11-1017G21.5 | MAGOH | DTX2 | SHISA3 |
| CHL1-AS2 | FAM109A | KLHDC8B | RP11-235E17.2 | TWF2 | CRTAM |
| RP11-262H14.5 | TTYH3 | AC114730.11 | FBXO7 | TMEM74B | NRF1 |
| RP11-376N17.4 | RP11-191L17.1 | AXL | BCLAF1 | DPH3P1 | RP11-5C23.2 |
| C9orf116 | ZNF444 | KAT6B | RP11-406H23.2 | YOD1 | CSAD |
| LDHA | CASP3 | VPS13D | IARS | TCP11 | ICA1 |
| RP1-101A2.1 | RPARP-AS1 | PPWD1 | MRM1 | CTD-2510F5.4 | AC004453.8 |
| ATP1B1P1 | SFI1 | TPRKBP2 | CBFA2T2 | LPP-AS1 | LSM8 |
| RP11-417F21.1 | RP5-890O3.3 | TMEM266 | TRIO | ARHGAP22 | AP000569.9 |
| CFH | AC009120.11 | HAPLN2 | OSCP1 | NXT1 | AC068831.16 |
| C6orf48 | SLC25A5-AS1 | DYNC1I2P1 | UBAP1 | CYFIP2 | NNT |
| DMC1 | ZCCHC5 | INIP | CTB-50L17.16 | MAPK9 | KIF7 |
| STAG1 | PTPRN2 | RPS7P3 | FSCN2 | FAM189A1 | RDX |
| FBXO22 | RP5-881P19.7 | TSPYL1 | SLC30A9 | PREP | ITPR1-AS1 |
| SPACA6P | SEC16A | TRMT2B | CCDC144NL-AS1 | LINC00479 | COPG1 |
| RP11-797A18.6 | KBTBD4 | RP11-34P13.16 | HAVCR2 | RP4-796I17.5 | RP11-214K3.21 |
| EIF4A2P1 | ACTL6A | MOGS | DNAJB9 | BX842568.2 | RP5-940J5.3 |
| RP11-61L19.3 | RPL28 | CDS1 | CAPRIN1 | LINC00958 | RP11-160E2.19 |
| SERF1B | RP11-481J2.4 | PPP1R36 | RP11-275I4.1 | SLCO3A1 | YBX1P2 |
| UQCRBP1 | PRKX | USP6NL | OAF | GPRC5A | UBXN7 |
| TMEM39B | BMS1 | UBXN2A | RP11-334L9.1 | LAGE3P1 | FBXO11 |
| ATR | STXBP1 | RP4-680D5.2 | SAPCD1-AS1 | AC006116.20 | DNAJB2 |
| BRD3 | PHLDB3 | ARMCX4 | SHPRH | CEP76 | TSNAXIP1 |
| RABAC1 | RAB40A | TOP3A | FERMT2 | CTC-308K20.1 | DEPDC5 |
| RPL39 | RP11-524N5.1 | PXT1 | FHOD1 | RPL23A | GAS6-AS2 |
| RNLS | RP11-511H23.2 | ZNF709 | RP11-422P24.9 | PSMB10 | ZNF891 |
| RP1-293L8.2 | FBXO30 | RP11-15B24.5 | TMEM8B | HSPE1P2 | BRD1 |
| KLRC1 | RAB9A | NDUFA4L2 | AD001527.4 | BCL7A | ARID4A |
| AC007228.9 | ZFAND2B | GRAMD4P8 | ATF5 | B3GALT6 | RP3-406P24.5 |
| RP11-73K9.3 | ENO1 | GGCX | UBXN6 | RAB18 | AC007773.2 |
| HOXA-AS3 | NELFCD | RARRES1 | ZMYM2 | RP11-456H18.2 | C14orf80 |
| AC078899.3 | CACNA1I | PLCG2 | SIGLEC16 | PIK3IP1-AS1 | NUDT7 |
| DUOXI | AP000295.9 | HSPA14 | ATG101 | SYCP3 | RPL15 |
| ACTR6 | NDUFB1 | CCNF | ZNF92 | RP11-298E9.7 | EIF4A2P2 |
| ZNF718 | SYCP2 | ITGA7 | IMMP1L | KCNQ1OT1 | CRHR2 |
| BRD2 | LINC00222 | NCOR2 | C1QTNF9B | FOXP1-IT1 | LINC00334 |
| BST1 | RP11-70C1.3 | JADE1 | ERCC5 | CTB-167B5.1 | TBC1D3B |
| MRPS17P1 | RANBP1 | CASP16P | CTD-2378E12.1 | RP5-1115A15.1 | UBE2N |
| RP11-483I13.5 | ACVR2A | HCG11 | ASPHD1 | RP11-473C18.3 | FAM103A1 |
| TAB1 | FAM162A | RGL3 | MC1R | RP11-104G3.2 | ADORA3 |
| JAKMIP3 | RPL10A | SKP1 | ZNF32-AS2 | RPL36A | CH507-9B2.2 |
| RP11-333E13.2 | AC016683.5 | C21orf62 | RPTOR | RP3-439F8.1 | FNDC9 |
| ATP11B | CCDC91 | KLF15 | CLLU1 | RP11-91K11.2 | AC092573.2 |
| FAM73B | ERBB2 | RSRP1 | C9 | NGFR | AC098826.5 |
| CCDC92 | CICP7 | RP1-78O14.1 | ZNF195 | TMSB4XP6 | AC008746.12 |
| RP11-613M10.6 | LURAP1 | SQRDL | ECHDC3 | CXCR4 | TMEM8A |
| URAHP | SMIM10L2A | RPL13AP7 | FAM105A | RP11-521L9.2 | FAM196B |
| NRXN2 | SEPT7P2 | TOX4 | ZNF189 | ENTPD8 | RP11-43D2.2 |
| SEPT4-AS1 | TMEM184A | SMARCD1 | YBEY | PSMA1 | PCDH1 |
| DDX6 | RP1-140A9.1 | FAT1 | MSL1 | RP11-15N24.4 | RP11-92K15.3 |
| USP54 | MED6 | TBC1D8B | PDCL3P4 | RP5-1068H6.6 | WIPF1 |
| RSPH1 | FUT2 | PLBD1 | RP11-1415C14.4 | ATRX | TRPV3 |
| RIN1 | C1QB | DPH7 | SERPINA5 | C16orf87 | CAST |
| WASL | PXMP2 | RP11-147L13.15 | CFHR1 | SETP3 | PNKP |
| AC012074.2 | PPCS | RCCD1 | RPS5 | NDUFA2 | MEF2B |
| CTC-281F24.1 | DICER1 | UGT3A2 | TMA7 | NEK5 | DLEU7 |
| ABCA17P | DAP3P2 | RP11-848G14.5 | SERPINC1 | BYSL | ZNF766 |
| RP11-138I1.3 | RBP4 | DEFB1 | CTC-459F4.3 | ZNF160 | METTL15P1 |
| INTS7 | TRIM5 | PITPNC1 | RP11-367N14.3 | RP11-37N22.1 | KRT8P33 |
| ATP2A2 | ANP32AP1 | G6PC3 | RP11-523O18.7 | AC114730.3 | RP1-225E12.2 |
| RP11-541M12.6 | RP4-751H13.5 | RP11-278A23.1 | C1QTNF3-AMACR | SRSF6 | RP11-157K17.5 |
| ELF1 | TYMS | RP3-467L1.6 | RP11-391L3.1 | RP11-849H4.2 | CYB5A |
| BRD7 | LINC00959 | PCED1A | RP11-631N16.2 | RP11-92K2.2 | DNAJC21 |
| RP11-510M2.2 | RP11-188D8.1 | LEMD2 | PEX6 | LBX2 | TTC9C |
| ID2-AS1 | FAM225A | RP11-42I10.1 | FNDC3A | RP11-11N7.4 | VN1R82P |
| PPP1R8 | TMEM223 | PCDH12 | PIK3CG | RP11-38M8.1 | TRIM27 |
| RP11-148B6.1 | CDK2AP2 | RP11-195F19.29 | SRSF1 | ELP6 | SNRPGP4 |
| RP11-817I4.1 | FAM149B1 | PGRMC1 | WNK3 | RP11-227D13.4 | USPL1 |
| HIST1H2BH | AC012456.4 | KCNK7 | AC005481.5 | RP11-445F6.2 | SMIM13 |
| RP11-264I13.2 | RP3-416J7.4 | PCBD2 | CTD-2270L9.2 | RP11-687E1.2 | RP3-508I15.10 |
| GINS3 | RP11-554E23.2 | F2 | RP11-565F19.2 | RP11-618G20.1 | ITGBL1 |
| LL0XNC01-250H12.3 | CTD-2561B21.7 | RP11-223I10.1 | RP11-15H20.2 | CILP2 | RP11-74C1.4 |
| AP000343.2 | VN1R74P | IFNL3P1 | RP11-105C19.1 | RP11-54G14.1 | RP11-648K4.2 |
| SPO11 | FBXO17 | NLRP11 | FAM171A2 | RBSN | RASA4DP |
| AC000068.10 | GLYATL2 | RING1 | AC005355.3 | RP11-27K13.3 | EIF4HP1 |
| ATP2B2 | MATN1 | EP400 | C20orf27 | C9orf129 | LINC00896 |
| FKBP2 | RPS19 | CTNNBL1 | PRDM12 | WIPF3 | IP011 |
| HIF1AN | AC004623.2 | ABHD17AP1 | HSF4 | CTD-3025N20.3 | PACSIN3 |
| ENG | DDX27 | HMGB1P24 | CLIC4 | ICE1 | AK4 |
| HCG17 | AC108868.3 | ZNF626 | SLC44A5 | RP4-595K12.2 | PRR29-AS1 |
| ARHGDIB | EYA3 | ADAMTS20 | RP11-343C2.10 | GAS2L2 | ATP6V0A2 |
| DUXAP8 | TIMELESS | RP11-299H22.6 | PDE6B | ULK3 | LDHC |
| LINC00882 | RP11-66D17.5 | LIG3 | RP11-379F4.4 | CTD-2562J15.4 | AC007036.6 |
| RP5-844F9.1 | SUGT1P3 | UBE2H | ASH2L | LPAR5 | ALB |
| SYT15 | CHRNB4 | AC105339.1 | ZNF527 | MYH11 | SEC24B |
| RNF224 | AL590763.5 | CTB-31O20.8 | OTUD5 | RP11-295D4.5 | C1orf21 |
| PHIP | SREBF2 | ITIH2 | FAM53C | SEC62-AS1 | CTD-2015B23.2 |
| VMAC | RP1-140C12.2 | RP11-394B2.1 | RP11-109E12.1 | TEN1-CDK3 | AC104534.3 |
| FOXJ3 | CTD-2310F14.1 | AC092881.1 | RP11-96D1.6 | CTB-133G6.1 | SMARCAD1 |
| RP11-44F21.3 | AC005037.3 | RBPMS2P1 | ATP11AUN | USP48 | LINC01543 |
| ATP11C | TRIM35 | MTND6P5 | LEFTY1 | RP11-715F3.2 | RP11-230C9.4 |
| HMGN1 | PLAC9 | RP11-80P20.3 | TAS2R43 | RAB10 | DHRS9 |
| GRID2IP | SUMO1 | MAPK8IP1 | RHOT2 | LINC00476 | RP11-31F15.2 |
| SNRPEP2 | GAPDHP65 | EIF4ENIF1 | RP1-30M3.6 | UPRT | TGIF2P1 |
| RP11-106M3.3 | GGCT | bP-2189O9.3 | MANSC4 | SMURF2 | RP11-512M8.11 |
| ANXA5 | RP11-354M20.3 | NFASC | FLU | RP11-84D1.1 | ZNF12 |
| SIAH2 | RP11-142C4.5 | CGNL1 | PTOV1 | CCNB2P1 | BRD7P4 |
| SEPP1 | AP000640.2 | AMZ1 | RP11-401P9.4 | HIST1H4B | EEF1A1P30 |
| RNF207 | CCR9 | SETX | RP11-462D18.2 | AC006539.3 | AP4B1-AS1 |
| RP11-22P6.2 | AHCYP2 | AP3D1 | C3AR1 | PSMA5 | IFFO2 |
| FMO1 | ATRIP | CTHRC1 | PPP2R3C | EIF5A2P1 | MSH2 |
| RP1-159A19.3 | MED16 | AC096558.1 | ORC1 | GALK2 | SKIV2L2 |
| FAM231A | RP11-18B3.3 | GSDMB | CNTNAP3 | BCL2L2-PABPN1 | HDAC8 |
| PATL2 | CTD-3065J16.9 | IFT74-AS1 | UGP2 | SLC10A7 | RDH10 |
| HSP90AB1 | PSMC1P1 | TNFRSF1B | PPM1D | FGF7 | RP11-872D17.4 |
| ZCCHC7 | STAG3 | CHP1 | RP11-225H22.7 | RP11-572M11.4 | RP13-1032I1.11 |
| RP11-956A19.1 | AC007283.5 | RP11-223A3.1 | HOXC-AS2 | CCT8L1P | RP11-483I13.6 |
| GAR1 | BOLA2B | RP11-305L7.7 | BMS1P20 | RP11-996F15.5 | RP11-488C13.1 |
| RP4-555D20.1 | DHX15 | RP13-991F5.2 | MRPL3P1 | GPR84 | RP11-299P2.2 |
| RPL7AP6 | FAM132A | RP11-284A20.2 | P2RY4 | LONRF2 | RPL13P4 |
| KLK14 | RP11-314N13.10 | SLC11A1 | NFKB1 | CSDE1 | RPL21P10 |
| FHL2 | AC007405.4 | FAM207A | AGGF1P2 | SLC5A12 | CAP2P1 |
| RP11-73E17.2 | OSTC | RP11-25D3.1 | OTOA | RP11-146E23.2 | NUP43 |
| YTHDF1 | ZNF252P | DDX25 | MFSD2B | RP11-182J1.3 | DNAH10OS |
| RP11-352G9.1 | AIM1L | EFCAB6 | DISP1 | WRNIP1 | EBF3 |
| TOR1B | RP1-122O8.7 | TSR2 | AC239868.1 | RP11-434I12.5 | SNX18P13 |
| IGHG3 | ALAS2 | RP11-605F14.2 | SRGAP1 | TAS2R31 | ZFP1 |
| AC004490.1 | LINC00598 | NCBP2-AS2 | RP11-317B17.4 | RP11-79N23.1 | SUN3 |
| AC093495.4 | GABPA | RP1-309F20.4 | FAHD2A | RHEBP1 | CLUH |
| SLC2A7 | SF3B2 | RP11-454E5.4 | RP4-705D16.3 | RP11-647O20.1 | LINC01297 |
| NCAPG | HSFX2 | CTD-2547L16.3 | TPM3P6 | RP11-49I4.3 | AF224669.3 |
| CFL1P1 | AL109761.5 | EIF4EBP2 | CTD-2621117.3 | RP11-48B14.1 | RP13-131K19.6 |
| NACA2 | HTR5BP | TP53TG1_1 | CH17-13I23.3 | RPL7P17 | RP11-430C7.2 |
| HSPH1 | RP13-317D12.3 | RP11-118E18.4 | GEM | CTD-2575K13.6 | PRDX3P2 |
| TET3 | SMC1A | FAM69A | SMTNL1 | DFFBP1 | RPL7P4 |
| RP11-638I2.4 | FUT3 | RP11-693M3.1 | RP11-215P8.2 | TMEM168 | RP11-244J10.1 |
| TRGV5 | SH2B2 | ARID3C | ECHDC1 | JAM2 | RP11-466F5.6 |
| TBC1D30 | CRYM | MLH3 | CEP85 | SIGLEC5 | CTC-436P18.1 |
| CTC-559E9.6 | SPATA45 | VTA1 | HEPHL1 | HNRNPCP4 | RP11-370I10.12 |
| DDIAS | SNRPE | CCM2 | RP11-219B4.7 | KIAA1211L | AL133493.2 |
| RP13-270P17.1 | B3GALNT2 | RP11-74E22.3 | STEAP3-AS1 | RP11-567M16.3 | RP11-273B20.1 |
| RHOXF2B | HIST1H2BM | AC019172.2 | CX3CR1 | MED20 | PDE6G |
| RP11-310P5.1 | SYNCRIP | RP11-234B24.2 | RP11-108P20.2 | RP11-235E17.4 | ING5 |
| RP5-827C21.2 | HNRNPA1P48 | MFSD6 | RP11-345P4.6 | RPS7P4 | RP11-1102P22.3 |
| RHOQP3 | ICMT | OPN1SW | RP11-358B23.7 | MAGOH2P | RANBP9 |
| PRSS8 | RP11-1017G21.6 | RP11-417N10.3 | YY1P1 | SGSH | GFOD2 |
| BATF3 | MORF4 | UPF3AP2 | CIAPIN1P | EBLN3 | RP11-157H4.1 |
| RP11-474B12.1 | KRTAP5-7 | ZNHIT6 | SLC2A2 | HIGD2B | SAA2 |
| RCC2 | RP1-256G22.2 | ERP29P1 | MEGF9 | RP11-396C23.4 | C1QBPP2 |
| RP11-126F18.2 | TRAM1 | RP1-127B20.4 | MED28P7 | RP11-217B1.2 | ST6GAL1 |
| LIMS1 | PRELID1P3 | ST8SIA5 | ACTA2 | MAP2K3 | NIT2 |
| INPP5K | NTN5 | OLFM5P | AC005154.7 | RP11-477I4.4 | CTD-2047H16.3 |
| SLC25A22 | AC010980.2 | CAMTAI | AP001429.1 | LHX3 | LINC01358 |
| RANBP6 | RP11-488L18.3 | NDRG3 | ARPC4-TTLL3 | PDPR | C14orf2 |
| RP11-220I1.5 | CTD-2262B20.1 | RP11-128A17.2 | THOC7-AS1 | SDR42E2 | ABCC8 |
| TBK1 | AC079753.4 | MID1 | DHX34 | MTND4P9 | BSN-AS2 |
| RP3-467K16.4 | RPS4XP6 | RP11-254F19.4 | SRRT | LINC01314 | RP11-51J9.5 |
| CECR5-AS1 | P2RX4 | ZC3H7A | RP11-1348G14.1 | C10orf55 | KB-318B8.7 |
| TRIM62 | RP11-146F11.5 | RP11-48G14.1 | RP11-392O18.2 | RP11-102G14.1 | MORN1 |
| LL22NC03-22A12.9 | GTF2IRD1P1 | RP11-506H20.2 | RP11-372B4.3 | RP11-365O16.6 | METTL21B |
| SREK1IP1 | ACTR1B | RP4-777L9.2 | RP11-367N14.2 | CSRP3 | COMMD1 |
| NDUFB11 | C3 | LINC01252 | RP3-514P16.1 | EIF4BP2 | HIF3A |
| RP1L1 | HNRNPA1P54 | GAL3ST2 | PIN4P1 | ISY1-RAB43 | RP11-849H4.4 |
| CNOT6LP1 | RP11-51L5.5 | RARRES2 | RP13-383K5.4 | AC009970.1 | LINC01410 |
| PNPLA6 | RP11-701H24.3 | CTD-2623N2.11 | HGF | U91328.19 | RP5-875O13.1 |
| NCKAP5 | BTN2A1 | CTD-2179L22.1 | LINC01301 | TRAF3IP2 | RP5-1031D4.2 |
| RPS15AP11 | LAMA2 | ZNF507 | HNRNPA1P61 | ACTA1 | SLFN12 |
| BARD1 | RPL32 | GLOD4 | RP11-104H15.8 | IPO8P1 | SETP22 |
| LL0XNC01-116E7.4 | RHOT1 | RP11-715J22.2 | WDR45BP1 | CTB-96E2.3 | GOLGA6L3 |
| EIF5AP3 | CTB-175E5.7 | HNRNPA1P4 | CTD-2308G16.1 | MED24 | SMG1P1 |
| CTD-2002J20.1 | TMEM74 | RP11-567C2.1 | RP11-57G10.8 | RP11-452L6.1 | AC006145.4 |
| LINC00652 | PIKFYVE | LINC00641 | TMEM97P1 | RNF103 | RP11-192H23.6 |
| C15orf61 | FOXL2NB | EEF1D | ANGPTL5 | SECISBP2 | CNDP1 |
| LCK | F13A1 | ZNF296 | RP11-299G20.2 | RP11-20B24.6 | RND2 |
| RPS18 | CTC-308K20.3 | GAPDHP1 | AP000925.2 | DHX35 | EFCAB9 |
| COL12A1 | ZNF833P | RP5-1173I20.1 | ZNF117 | TCF12 | AP000354.2 |
| RP11-894J14.2 | CFAP69 | CTD-2349P21.11 | GPR173 | NIPAL1 | CTA-407F11.8 |
| TEX12 | CSF1R | MRPS31P5 | FEZ1 | C19orf35 | RP11-791M20.1 |
| AC020594.5 | RP11-703I16.1 | AKAP17A | AKAP17A | RP11-338E21.1 | AL928742.12 |
| CTA-972D3.2 | RP11-643G16.3 | WFDC2 | DUSP22 | CTSL | LINC00239 |
| RP1-121G13.3 | EEF1E1P1 | MYB-AS1 | VTI1BP2 | ANKRD20A17P | GCNT7 |
| CTA-440B3.1 | UTS2 | RP1-182O16.2 | RP11-37C7.3 | ZNF354A | RP11-597M12.2 |
| HP09025 | CTC-510F12.2 | RP4-736L20.3 | MITF | UBE2CP1 | CTD-2583A14.11 |
| RP11-58O9.2 | CNTN2 | KLRC3 | CNTROB | NUP210 | RP11-696L21.2 |
| DLGAP1 | CATSPER2P1 | HMGA1P3 | RP11-303E16.9 | PDXDC2P | CDC20 |
| DGCR11 | DPP8 | RP11-723O4.7 | RP11-452G18.1 | RP11-425D17.1 | RP11-62J1.3 |
| FAM133B | AC084809.2 | VEZF1 | SNAP25 | RP1-5O6.4 | CAPN6 |
| SNAPC4 | SLC9A7P1 | FAM167A-AS1 | POT1 | SYCE1L | RP11-302B13.1 |
| CDH26 | HABP2 | ZNF646 | AK3P3 | RN7SL809P | C15orf48 |
| PPIAP22 | TRBC1 | RP11-430C7.5 | CCNL2P1 | CD200 | GOLGA1 |
| FBXL20 | RP11-713N11.4 | RP11-119H12.4 | SIDT1-AS1 | RP11-148O21.4 | HNRNPUL2 |
| RP11-572O6.1 | RP11-350J20.9 | RP11-2C24.3 | RP11-252I14.1 | ELP2 | RP11-263F14.3 |
| RP11-2H3.7 | PLB1 | DGCR5 | RP11-91A18.4 | KIAA0368 | PCNXL3 |
| HNRNPA1P66 | THAP5P1 | JARID2-AS1 | AC016629.3 | PLEC | RP11-109E24.2 |
| AP2A1 | NXN | VPS29 | RP11-77M5.1 | NME2P1 | UBE2E1 |
| GPKOW | RP4-730K3.3 | ZNF644 | KRT18 | GBGT1 | POLR1D |
| BDNF | EIF4EP2 | FABP5P3 | TRAPPC4 | CTSC | EIF2B1 |
| RP11-162A12.2 | RPLP0 | RP11-363J20.1 | ACTG1 | PRPF8 | C9orf41-AS1 |
| BASP1 | TCEB3B | RP4-761J14.9 | MYO1E | CTC-523E23.4 | SNHG15 |
| SARS | FBXL12 | RP11-1228E12.2 | SLMAP | PCDHGB6 | SNHG9 |
| GNG10 | RNF216 | RP11-445P19.3 | RP11-285F7.2 | TARSL2 | PDXP |
| CICP5 | ALG1 | SS18 | NUP107 | CTC-287O8.1 | RP11-1060G2.2 |
| TUBE1 | CTC-435M10.12 | CYP4F3 | TH2LCRR | RP11-460N20.7 | TTLL7 |
| KIAA0825 | CTA-212D2.2 | RP11-266K4.14 | CTA-113A6.2 | ZBTB20 | RP11-11M20.2 |
| EIF2B5 | KRTAP5-8 | XRCC5 | RP13-614K11.2 | DBP | AC005562.1 |
| ITPA | CAPZA1 | PEBP4 | RP11-439A17.9 | RN7SL219P | ZNF691 |
| NACC1 | RP5-1000K24.2 | GNAT2 | RP11-167N4.4 | RP11-357N13.2 | OVCH2 |
| OLA1P3 | RP11-346E8.1 | IFT80 | ITPK1-AS1 | CYP27A1 | FAM92A1 |
| RPS3P2 | SGMS2 | GRASP | PLK4 | COX7BP1 | SHISA7 |
| ATF4P4 | SPAG5-AS1 | RFPL4AL1 | RP11-1277A3.1 | AC013264.2 | RP11-116K4.1 |
| RPEL1 | RPS4XP3 | AL022578.1 | BAMBI | HNRNPF | PTCD1 |
| RP3-324O17.7 | MAGI3 | RP4-713B5.2 | IGHV1-69 | RP11-9L18.2 | LINC00487 |
| RP11-173P15.10 | COPB2 | RP11-96C23.11 | TMEM59L | RP5-1074L1.1 | RP11-277P12.10 |
| TGFBR3 | TLR8 | CFI | TIMM17B | H1FNT | RP11-557C18.3 |
| HNRNPA1P35 | AC005624.2 | AC124914.3 | RP11-248M19.1 | TAS2R10 | RP11-734E19.1 |
| IGHV3-79 | UGT2B7 | ATP2B4 | UQCRB | RHCE | CTC-338M12.5 |
| RP11-553L6.5 | EVI2A | RP11-188C12.2 | KCNE2 | NUP214 | PLAUR |
| CHMP1B | RP11-368I7.4 | GS1-393G12.14 | PLIN4 | HMGB1P10 | RP6-109B7.4 |
| RP11-524O24.2 | PTOV1-AS2 | KEL | GTF2IP9 | RP11-1277A3.3 | RP11-989E6.10 |
| RP11-108F13.2 | CCT6A | C9orf91 | SUPT16H | DGKH | MTA1 |
| FBXL8 | BNIP3P39 | AL591893.1 | RP11-102M11.1 | CCL2 | RP3-510O8.4 |
| DCUN1D3 | RABGEF1 | RP11-680G24.6 | XXbac- | CRACR2B | SUCLA2-AS1 |
| | | | BPG308K3.5 | | |
| AC008132.13 | RPSAP47 | HM13-IT1 | RP11-89H19.2 | CSNK1A1 | RP11-142L4.2 |
| FAM47E-STBD1 | ZNF280D | ID4 | MEAF6 | SERPINA7 | ACBD3 |
| IRF8 | HNRNPA1P2 | IL1A | SNORA53 | ARHGAP15 | DNAJC19P5 |
| BMP3 | RP11-109P14.9 | RP11-130F10.1 | LENEP | NDUFB8P2 | GSG2 |
| RP13-16H11.8 | PLEKHA8P1 | PFDN1 | RP3-337O18.9 | ALOX12B | ACTL7A |
| RP11-413H22.3 | AC073283.7 | SLC7A2 | STIP1P3 | PTAR1 | NDUFB3 |
| ARL13A | TK2 | NPHP3-AS1 | RP11-289H16.1 | INO80 | TACR1 |
| C7orf73 | FBXL13 | FAM104A | AK2P2 | RP11-523H20.3 | ANGPTL4 |
| RN7SKP110 | ATP6V1G1 | RP11-71H17.1 | RP3-395C13.1 | KIF5A | SLC13A4 |
| RP11-973D8.5 | RNF183 | AC002310.11 | CTD-2371O3.2 | SNORD94 | RP11-126K1.2 |
| IGHV1-69-2 | RP11-1012A1.7 | LINC01364 | RP11-133N21.7 | TAS2R3 | RP1-90J20.8 |
| YRDCP2 | CTD-2252P21.1 | STX1B | RP11-544A12.8 | BEND6 | SERPINA1 |
| RP11-55K22.2 | RP11-67A1.4 | PPP4C | RP11-46A10.8 | ZNF253 | PANDAR |
| ENTPD4 | BACH1-IT2 | FMN1 | PPFIBP1 | VIPR1-AS1 | RP11-178C3.6 |
| ADGRF3 | RP11-272L14.2 | AC139100.4 | MRLN | AC006486.9 | RP11-501C14.7 |
| DDX47 | OSR2 | WI2-89031B12.1 | UNC93B3 | RP11-611O2.1 | IGBP1-AS1 |
| RP11-98G7.1 | NTSR1 | LPAR3 | JAZF1-AS1 | LUCAT1 | DYX1C1 |
| MIR3180-4 | FAM209A | CTC-523E23.1 | RP11-342K6.3 | RP11-229P13.23 | PEAK1 |
| RP11-709D24.6 | ST3GAL5-AS1 | ZNF733P | AF131215.3 | RP11-415J8.7 | TTLL12 |
| UBE2D3P1 | KIAA1429 | ODF2 | RP11-708J19.3 | FTX_5 | ISOC1 |
| DIMT1 | MINOS1P2 | EIF2AK1 | AC007560.1 | PPIL6 | TMSB4XP8 |
| SNORA41 | SNORA17 | SRD5A2 | RP11-981G7.1 | AIRN | AC011738.4 |
| RP11-613M10.9 | ZNF835 | RPS26P52 | RP11-95M15.2 | RP11-568G11.4 | RP11-474D1.2 |
| RCC2P6 | GBA3 | RP11-375O18.2 | RP11-423E7.2 | RP11-536C10.10 | ARL14EPL |
| OR13I1P | EIF5P1 | GCC2-AS1 | PGGT1BP1 | LINC00824 | RP11-314A15.2 |
| RP11-12G12.7 | FCAR | LINC01605 | TMEM14B | TTC17 | HMGN1P4 |
| RP11-535C21.3 | RANP8 | UBE2E1-AS1 | RP11-403I13.10 | RN7SL55P | CDC25A |
| COPS8P2 | RPS8P10 | RP11-983G14.3 | RP11-173A6.2 | THAP2 | RP11-123M21.2 |
| ZNF649-AS1 | RP11-582E3.6 | AC004837.4 | AMN | RP11-298O21.7 | RP3-403A15.1 |
| RP11-454K7.1 | ATP6V1G1P4 | CTD-3193O13.14 | SDHCP4 | RP11-15F12.3 | AC079305.10 |
| RP4-666F24.3 | NAA38 | RN7SKP249 | AC007292.4 | RP11-521C20.3 | CTA-280A3_B.2 |
| IGLV5-37 | CTD-2265O21.7 | RP4-764O22.1 | SUB1P1 | RP11-374M1.5 | CTD-3193O13.13 |
| LOC100421166 | IGLV4-3 | RP11-1365D11.1 | PARK2 | RP11-167N4.5 | RP1-34B20.4 |
| RP11-285A1.1 | BCAN | RP11-382A20.7 | RNASEH1-AS1 | FALEC | TEAD4 |
| AC073343.13 | RP11-393N4.2 | CTD-2534I21.8 | GALNT10 | RP11-680H20.2 | RP11-120B7.1 |
| RP11-290L1.2 | RPSA | RP11-292F9.1 | RP11-367E12.4 | KIAA1671 | TEX35 |
| CHI3L1 | RPL5P23 | FRK | NTN3 | RP11-33E12.2 | RP11-358B23.6 |
| FAM117A | RP11-329J18.5 | AC092651.1 | EMP1 | SRP54 | HTATSF1P2 |
| RP11-109E24.1 | SNX14 | KIAA2013 | LLPH | AC008280.3 | AC091878.1 |
| CAPZB | AQP1 | C6orf52 | RP11-425A23.1 | MESTIT1 | MTND1P9 |
| LPXN | FICD | PAGE2B | RAPGEF4 | ASPN | MORN2 |
| BCL2 | RP11-345I18.4 | MRC2 | ZNF335 | REM1 | RP11-16C1.2 |
| DRAXIN | U51561.1 | TMEM176B | PFN1 | CTD-2007H13.3 | NUP210P1 |
| BTBD1 | TNC | MYRFL | RP5-1024G6.7 | TRPC4AP | RP11-673E1.3 |
| CCSAP | ANKS3 | EEF1B2 | LINC01287 | SLPI | AC024937.2 |
| RPL21P4 | RP11-367J11.3 | XX-FW81066F1.2 | RP11-545M17.3 | CTC-329D1.2 | RP11-43N5.1 |
| TMEM176A | UFD1L | RP11-186B7.7 | HOPX | RPSAP61 | CHRNA7 |
| CDC42BPG | SPRED2 | COX17 | RPS2P45 | SARDH | LINC00861 |
| POLR2A | MAMSTR | RP11-1136G4.2 | AMMECR1L | AC005042.2 | LMTK3 |
| AC005307.1 | IST1 | GRID1 | TMEM154 | SNORA73B | AC016738.3 |
| RP11-253I19.3 | RP11-18H21.1 | TMEM140 | CTD-2410N18.5 | UBALD1 | KIF18B |
| ADAD2 | DHX36 | RP11-762H8.1 | CDH24 | KLF7 | DCST1 |
| AS3MT | RP11-693J15.5 | CCDC73 | CNTLN | RBM20 | RP11-484L8.1 |
| POLR1B | LINC01555 | NSF | BTF3L4 | RP11-530C5.2 | TPT1P4 |
| CTA-963H5.5 | TMC3-AS1 | CTD-2329C7.2 | EEF1A1P4 | RP11-365H22.2 | RP11-707G18.1 |
| NPIPB3 | CAMK2N1 | SNRPCP3 | RP11-731J8.2 | RP11-597D13.7 | GS1-259H13.7 |
| RP11-1017G21.4 | NKAIN1 | RP11-495P10.5 | SNPH | RP11-677O4.6 | STAT5A |
| LINC00909 | PGM3 | MICU1 | RP1-20C7.6 | HIST1H4F | RP11-440D17.3 |
| PPIF | THAP9 | RP11-764K9.1 | NSUN5 | TNFRSF12A | BIN2 |
| TRADD | GRAMD2 | HIF1A-AS2 | HNRNPA1P36 | RPL18AP2 | AC008074.3 |
| RP5-867C24.1 | RP11-83B20.1 | ARHGAP5-AS1 | STARD6 | AC109333.10 | CHCHD7 |
| RP11-455F5.4 | NUTM2D | AC011816.1 | FAM90A1 | PABPC1P1 | AC132217.4 |
| CTD-2600O9.2 | EYS | HSD17B13 | RP11-197N18.8 | SGF29 | TMEM170A |
| RP11-321F8.4 | RP11-511H9.3 | GRHL1 | NEK1 | MOAP1 | RP11-522M21.3 |
| ANPEP | DLEU7-AS1 | COBLL1 | RP4-791M13.3 | NPAS3 | HEYL |
| SEMA6A | EHHADH | TMEM236 | DTNBP1 | RP11-798G7.7 | GOPC |
| ZNF205 | RN7SL211P | KRTAP5-2 | AP003068.9 | RN7SL181P | RP11-1100L3.7 |
| AC012314.8 | CTD-2528L19.3 | SLC1A3 | CDC7 | RP11-15B17.1 | PPP1R2P3 |
| RP11-265N6.2 | PTX3 | RP11-61L19.2 | HYI-AS1 | CLTA | H19 |
| SEPT14P12 | RPS12P20 | FAM209B | DMRT3 | AL357140.1 | CTB-12A17.2 |
| ZNF83 | MCM5 | WDR5B | SYVN1 | RP11-285E23.2 | MRPS34 |
| Sep-10 | ENPP7P12 | FGF18 | HYDIN | RP11-204M4.2 | RP11-315D16.2 |
| SETP5 | KLHL15 | STX17-AS1 | EFTUD1P1 | TMEM45B | BLM |
| RP11-763B22.3 | ACTB | RP1-274L7.4 | ZNF549 | SATB2-AS1 | ERV3-1 |
| SNRPB2 | SPATA6 | RPS12P31 | RP11-129K12.1 | RP11-271C24.3 | MN1 |
| RP11-814H16.2 | TRIM72 | FAR2P2 | AC100830.4 | ETFDH | GK-AS1 |
| C7orf49 | PIK3C2B | ADAM11 | PYM1 | CAV1 | PHBP13 |
| THOC6 | DEF6 | CCDC58P3 | OSBPL5 | FMR1-AS1 | CHTF18 |
| RP11-23P13.4 | PLAA | SPATA6L | LAS1L | RP11-64B16.2 | ZNF720 |
| CDH1 | SETP20 | PRDM8 | RPL37P23 | RP11-353K11.1 | MCM8-AS1 |
| CDH4 | LYPLA1P3 | TGM4 | AC006004.1 | C11orf94 | CLP1 |
| RP11-130C19.3 | RP5-1042K10.13 | HCG4P3 | TFCP2 | PDE1C | YIPF3 |
| R3HDM2 | AC013470.6 | RP11-193F5.4 | SHISA4 | RPL4P4 | RP11-537I16.2 |
| ENAH | DDX52 | PIR | FOXD4 | RP11-45M22.4 | CROCCP2 |
| RPL21P116 | RARRES3 | RP11-166N17.1 | RP1-53O8.2 | KRT8P50 | LINC01342 |
| EMP2 | RP11-206F17.2 | GS1-393G12.12 | ELMO3 | CYP26C1 | CTC-510F12.7 |
| RP11-24C3.2 | RBX1 | MAP1LC3B | RP11-666A8.8 | CTD-2008P7.6 | GTSF1 |
| RAD51C | STK33 | DGCR9 | ABHD14A-ACY1 | AMZ2P2 | PDCD1 |
| CTD-3214H19.4 | CTD-2095E4.4 | SLC17A5 | OR6B2 | FMR1NB | ITPKB-AS1 |
| GC | LL21NC02-1C16.1 | AC007238.1 | RBBP4P4 | RFTN2 | HDAC6 |
| NKX3-2 | PRKCI | FRAT2 | PCDHB13 | RP11-406A9.2 | ZZEF1 |
| CSDAP1 | SNRPD1 | ISX | RAB2A | KLF3P1 | AC097721.1 |
| DDX5 | WBP11 | CSNK1D | RP11-402D21.2 | RP4-639F20.1 | ZC3H12C |
| RP5-1031D4.3 | AC025918.2 | RP11-80H5.9 | RP3-412A9.17 | AC004967.7 | APOH |
| LINC01444 | CCNE1 | AP1B1 | RP11-17M16.2 | EDC3 | RP11-305B6.1 |
| CST3 | ETF1P2 | LL22NC03-23C6.12 | RP11-603J24.5 | STXBP5-AS1 | RP11-271M24.2 |
| CXCR6 | RP11-390B4.3 | RP3-403A15.5 | LINC01298 | ANAPC10P1 | CTD-2292P10.4 |
| MEPCE | RP11-403P17.3 | NCDN | POLR2M | RP11-778D9.4 | ZNF480 |
| NFE2L3 | MED8 | CKS2 | EPS8 | CTD-2192J16.11 | TEX19 |
| AC007386.3 | SMCHD1 | RTN4R | SLC4A1APP1 | RP11-527H14.4 | RP11-386G11.3 |
| RP11-630A13.4 | AC017053.1 | ANXA2P2 | AC015688.3 | SNRPF | UPF3AP1 |
| TMEM178A | AF127936.3 | CDCA4P4 | ZNF75A | COL3A1 | MRPS16 |
| PTENP1 | RN7SL449P | NIFKP6 | RP11-430L17.1 | SMPD2 | UTP3 |
| RP11-686G8.2 | CCDC42 | ANKRD62 | EEF1E1 | WBP2NL | CTD-2135J3.3 |
| ZNF512B | LANCL1-AS1 | MT-RNR1 | MPZL3 | CENPQ | OR1F12 |
| VPS33A | CYP4F2 | KCTD20 | EIF4A1P4 | TUBBP5 | PPP4R2 |
| ADH1C | AC018737.3 | uc_338 | SLC25A3 | PPIAP6 | SCARNA9 |
| MEI1 | RASSF2 | AP000265.1 | RP11-455J20.3 | RPS15AP10 | RP11-156L14.1 |
| RP11-432J22.2 | PCSK1N | TMEM169 | ZKSCAN7 | CDK1 | LARS |
| CH17-302M23.1 | RSL24D1P6 | TTC21B | RP11-91P24.7 | TSKS | HSD17B14 |
| CTC-303L1.2 | RPS19P1 | COG3 | RP11-597D13.9 | RP11-34P13.3 | GAS2 |
| NMRK1 | PCBP2 | PTPRS | AC020915.2 | MDS2 | MAST4-AS1 |
| PABPC1 | LINC00668 | RPL12P8 | PKN2 | CTD-2514K5.4 | RP11-195E2.1 |
| DNMBP-AS1 | CTB-179K24.3 | GUCY2C | RP11-386I8.4 | AC139103.1 | AP006285.2 |
| RP11-903H12.5 | RAB4B | CTB-35F21.1 | WRAP53 | LBP | GOLGA7B |
| RP3-330M21.5 | MTF1 | SUPT16HP1 | CTD-2555A7.2 | COL17A1 | UNC119 |
| ARPC3P2 | TAF5L | CTD-2647L4.5 | NODAL | CTC-436P18.5 | SLC2A3P1 |
| RPL34P18 | BRK1 | RP11-77H9.6 | ESRRAP1 | HHIP-AS1 | RP11-2E11.5 |
| MAEA | TBC1D10C | RPL23 | WDR53 | RN7SL316P | ZNF526 |
| JKAMP | RP11-321F6.2 | LUZP6 | AC005682.6 | SERINC5 | RP11-511P7.5 |
| AC128709.4 | MUC5B | NAP1L1P2 | RP11-525K10.3 | RP11-977G19.14 | AHCYL1 |
| EIF4B | TOMM70A | RP11-435O5.4 | SUCLA2P1 | ILF3-AS1 | CTD-2256P15.4 |
| RP11-423F24.3 | COX20P1 | PHF13 | RP11-840I19.5 | AC009502.4 | RPL35P2 |
| ZNF37A | SNX25P1 | TMEM240 | RNF169 | DDX39B | PDCD7 |
| AC079117.1 | RP5-837J1.4 | BACH1-IT1 | AC113188.2 | HIST1H3C | AP3S2 |
| ZNF586 | EDAR | OST4 | CTD-2135J3.4 | SMARCE1P6 | FGD6 |
| ADTRP | RP11-420K14.3 | RPS7P7 | SLC35F2 | SIAH2-AS1 | RP11-156E8.1 |
| SLC36A1 | TRIM29 | FAM107A | LA16c-361A3.3 | ASB9P1 | RP11-1102P22.2 |
| RP11-9E17.1 | RP11-774O3.1 | ZNF814 | CLCN3 | ORM2 | AC106782.20 |
| CENPA | ZNF267 | TGFB1 | KBTBD3 | RP11-278A23.4 | XXbac- |
| | | | | | BPG116M5.17 |
| ZSWIM1 | FAM58A | XPO7 | RP4-751H13.7 | GDA | SAA2-SAA4 |
| LINC00648 | MYL2 | RP11-1197K16.2 | RP3-355L5.5 | FEN1 | AC004980.9 |
| RTN4RL2 | RP11-216N14.7 | TFP1 | FAM151B | RP11-17G12.2 | EIF5AP2 |
| TMEM261 | AC009302.2 | RP11-567M21.3 | RPL4P5 | HINT1 | UGT1A6 |
| RPL34-AS1 | AJ003147.11 | AAR2 | ERI3-IT1 | CTD-2008E3.1 | HSPA4L |
| AC009487.6 | RP11-286E11.2 | U1 | ANP32C | RP11-890B15.2 | RP11-2C24.4 |
| DDX12P | SF3B5 | CCDC88C | OR11H7 | DDX19A | AIDA |
| RP11-529H22.1 | ZNF595 | COMMD2 | AC005578.3 | HNRNPUP1 | FXR2 |
| FGR | C1QTNF9B-AS1 | RP11-379F4.8 | VIPAS39 | PHLDA2 | ANLN |
| XXbac- | CXCL12 | RP11-94C24.13 | IGHGP | RHBDL3 | C8A |
| BPG170G13.32 | | | | | |
| TTTY10 | CENPF | PAX5 | ANAPC10 | RP11-97O12.7 | CTD-2104P17.2 |
| HOTAIRM1_2 | CTD-2517O10.6 | ZNF106 | LRRC75A-AS1 | NOM1 | CWC27 |
| uc_338 | RP11-3J10.7 | ARR3 | MELK | TCEAL3 | SKA2P1 |
| COL6A3 | RP11-6B6.3 | RP3-508I15.21 | MFSD7 | AC144530.1 | ADAP1 |
| GPR182 | CTD-3222D19.8 | SKP2 | CTC-260E6.6 | PIP4K2C | AC012531.25 |
| RPS6KA2-AS1 | PEX16 | COL4A4 | CTB-54O9.9 | PRELID1P1 | POTEKP |
| RP1-197B17.3 | ADCY10 | TMEM160 | TRMT1 | RP11-347C12.8 | C3orf67 |
| LEMD1 | BEST2 | ZNF202 | SLC6A16 | FBXL16 | BAALC-AS1 |
| CDK19 | CD14 | ALDH1A2 | RP3-417G15.1 | TMPRSS5 | BTF3P10 |
| UNC45A | ARHGEF7 | Sep-03 | Telomerase-vert | DACT3-AS1 | AC000068.9 |
| ARHGEF9 | RP11-43F13.3 | SPATA1 | IFNA21 | IGKV1D-43 | CRACR2A |
| BNIP3P22 | SPRY1 | HP1BP3 | RP13-467H17.1 | PPP1R27 | UBA3 |
| TSC1 | RP11-1069G10.1 | DIRC3 | VAMP2 | RP11-196G18.3 | RP11-330M2.4 |
| ALDH1L1 | PRKRA | LINC01037 | RP11-10J21.4 | AC018890.6 | ZNF721 |
| EIF2A | WDSUB1 | RP11-461L13.5 | FAM60A | ZSCAN22 | FAM207BP |
| GS1-25M2.1 | DUSP5P1 | DENND2A | RP11-343B5.1 | PHLDB2 | B2M |
| HNRNPA1P10 | RP11-65N13.8 | PTPRR | SNORA52 | HSPA8P9 | RP11-402J6.1 |
| MYL4 | AC009228.1 | MYL6B | HOTAIRM1_5 | NKIRAS1 | BHMT |
| MIIP | RP1-29C18.10 | RP11-421L21.2 | DAGLA | YPEL4 | AP000253.1 |
| CTD-2062A1.2 | TSHR | RAB28P5 | CCDC151 | PLXNB1 | PKMP1 |
| RP11-18I14.11 | TANC2 | LOH12CR2 | FZD1 | RP4-669P10.20 | RP11-972P1.7 |
| AC114776.1 | MRPL51 | MAN2B2 | NSMCE2 | MRGPRG-AS1 | NAT1 |
| RP11-2H3.6 | ZNF669 | SLC35E2B | CNPY1 | COL4A3 | CTD-2007L18.5 |
| RP11-44N11.3 | PNN | FBXW2 | RPL31P11 | TARBP2 | FFAR1 |
| RP11-266K4.9 | SH3GL1P3 | RN7SL78P | ZNF365 | CCDC152 | LA16c-390H2.1 |
| STRIP1 | KCTD4 | SRPK2 | RP11-197N18.2 | SHROOM1 | USP50 |
| SCNN1A | IPO7 | RPL7AP25 | MME-AS1 | TRIM56 | ENGASE |
| KHSRP | KB-1615E4.3 | RP3-337H4.10 | UQCRC2 | ATOH8 | CCDC121 |
| HLA-DPA1 | FAM60BP | HPS4 | RP11-409C19.2 | APBA1 | MZT2A |
| FAM166A | NPM1P21 | TNFSF12 | RP11-474P2.6 | NEK2 | DLEU2_1 |
| ROBO3 | LVCAT1 | RP11-466A19.1 | WDR91 | KDM6B | UPF3B |
| RP11-56D16.8 | LRRC75A | RP4-798P15.3 | KRT18P37 | NPIPA7 | RP5-1086K13.1 |
| FAM78B | CFAP52 | RP11-34P13.7 | RP11-537H15.4 | LCMT1 | Sep-02 |
| IGLJ2 | PPEF2 | NEUROD4 | MAMDC2-AS1 | IGF2 | RP11-424M24.5 |
| RP11-227B21.2 | MKNK1 | ASS1 | RP11-677M14.5 | SERBP1P6 | BFSP1 |
| RP5-837I24.1 | FGF14 | CTC-428G20.6 | CHRD | HMGN1P37 | ETV5 |
| AC025442.3 | RP11-27I1.4 | PAPPA | C1RL-AS1 | LARP4P | AP000302.58 |
| HIST1H2AL | ALG1L13P | GXYLT1P4 | CWC15 | RP3-369A17.6 | NANOGNBP3 |
| Sep-04 | CHRND | AP001059.7 | ZNF382 | KLHL14 | SPP1 |
| SLC35E1P1 | CEP89 | ADSSL1 | ZNF234 | RP11-187C18.4 | PVRL4 |
| TRIM54 | ADGRB1 | ZNF180 | RP11-274B21.10 | RP11-573D15.3 | MTUS2 |
| C21orf91-OT1 | HES7 | MARK2P4 | CPNE6 | RP11-446H18.6 | LINC00659 |
| AC015969.3 | CLU | SNAI2 | PHF20L1 | RP11-848P1.4 | AC016629.7 |
| MAP1LC3B2 | ZNF30 | RP11-227G15.2 | CD81-AS1 | ZC3H15 | CTAGE3P |
| SLC22A7 | DCT | METTL17 | CEP250 | CLEC18A | CHTF8 |
| TPR | NFX1 | ZWINT | FAM134B | RP11-339B21.13 | CNPY3 |
| RP11-294N21.3 | CCDC154 | RP11-631M21.7 | ATN1 | DYNLT1 | OSM |
| LAMB2 | RP11-60A14.1 | C3P1 | FTH1P5 | RNFT2 | BMS1P16 |
| RP11-44N22.3 | GPC4 | RBMS2 | LL22NC03-80A10.6 | RP11-229P13.25 | NR4A2 |
| AC007383.4 | RP5-944M2.3 | NTRK1 | TBX1 | RPL7P14 | EIF4A2 |
| ASGR2 | EXOC4 | RP11-477J21.2 | SLC47A1 | RELL2 | HLF |
| RP11-53B2.4 | TBCA | OR13E1P | CBLB | RP11-1136G11.8 | RP11-128N14.5 |
| CECR6 | RP11-732A19.5 | AP001631.10 | CH507-152C13.6 | ABC12-49244600F4.4 | Metazoa_SRP |
| PRPH | ERH | GAREM | H3F3AP6 | RP11-382D8.3 | HMGCS2 |
| DNMT3L | RP11-47A8.5 | DBH-AS1 | RP11-618K13.2 | PCTP | RP11-823E8.3 |
| GNG13 | RP11-90M2.5 | RP3-441A12.1 | GPS2P2 | USP13 | TCEA1 |
| AC023128.1 | KB-1027C11.4 | LARP7 | KCNMB2-AS1 | RP11-227G15.11 | GTF2A1 |
| GTF2B | PAX8 | PMS2 | MYO1F | CCNDBP1 | ZBED4 |
| SLC7A11 | EIF3G | CD248 | ZRSR2 | CHCHD2 | KRT8P39 |
| DLG3 | EIF4A1P2 | PFN4 | RBM4 | DZIP3 | NCLN |
| IGLV2-23 | CTB-43P18.1 | ADAM20 | EXOC6 | RP11-136K7.2 | RP11-290D2.3 |
| UGT1A7 | BGN | C20orf144 | RP11-22B10.3 | BPNT1 | PPP1R11 |
| DLG1-AS1 | TMEM150C | APEX1 | SRSF5 | PSMD3 | NRG2 |
| ATF1 | RP11-689P11.2 | CNGA4 | PGM5-AS1 | MT-RNR2 | MEP1B |
| AC108004.3 | CTD-2081C10.1 | EOGT | FA2H | AC003006.7 | AC110781.3 |
| RP13-46H24.1 | RASGEF1A | RPS25 | CCT5 | RP11-57H12.5 | RP11-219G17.8 |
| RP11-153M7.3 | CXCL8 | AC069368.3 | C17orf78 | PSMD10 | U1 |
| RP11-661A12.7 | U1 | BTBD8 | KLHL18 | STK4 | NECAP2 |
| CTAG2 | MR1 | CCNO | CTC-242N15.1 | TONSL-AS1 | C10orf91 |
| LRRN2 | CDHR2 | OR6L1P | RP11-160N1.10 | RP11-156E6.1 | ZG16B |
| SLC39A9 | UTP15 | CTD-2199O4.3 | FAM83E | GALNT15 | CUL2 |
| RP11-395I6.2 | TMEM17 | RNF208 | BCAM | PLEKHA7 | CTD-2O35E11.5 |
| UBA1 | RP11-136K14.2 | EPPK1 | TNFRSF8 | AIRE | CXorf58 |
| CREG1 | LA16c-60D12.2 | CTD-2083E4.6 | PLA2G12AP1 | RPS2P4 | PPP1R3B |
| FAM83G | CTC-459F4.1 | ADH4 | NAT10 | RP11-30L15.4 | FKBP10 |
| MZB1 | TIMP2 | STX7 | SMAP2 | SLC22A1 | HIST1H2AD |
| TMEM165 | CIR1 | BHLHA15 | LINC01398 | DCAF7 | GEMIN2 |
| PPP1R14B | DPYS | TSSC2 | AC099535.4 | POLR3E | RP11-565J7.1 |
| PIGB | AHNAK | BUB1B | B3GNT5 | ZNF429 | AP000350.5 |
| C14orf166 | SMIM3 | BAGE2 | XRN1 | ITFG1-AS1 | SNORD10 |
| KDELC2 | PPY2P | AC017104.3 | AC005754.7 | AC009963.3 | IWS1 |
| AC217773.1 | ZNF221 | RNU1-106P | FTX4 | RPL39P34 | TCL6_3 |
| RP11-318C24.1 | SURF2 | TLL1 | UQCRHL | HIF1AP1 | RP11-98F14.11 |
| uc_338 | ITIH3 | CACUL1 | LACTB2-AS1 | RP4-635E18.6 | IGLV3-24 |
| RP11-284A20.1 | CH507-42P11.2 | KIAA1522 | RP3-406P24.3 | SNORD91A | LIPH |
| RP11-885B4.1 | RP13-225O21.2 | RP11-571I18.2 | RP11-1250I15.1 | MTCO2P2 | RP11-262M14.2 |
| RP11-739N20.3 | RP11-1212A22.1 | RP4-758J24.4 | RP11-589N15.2 | SNORA70 | Y_RNA |
| SNORA10 | SNORA5A | SNORA14B | SNORA40 | SNORA24 | EXOC3L2 |
| FARSA-AS1 | GIMAP1 | IRS2 | PCDH19 | AF127936.9 | DSG3 |
| RP11-454H19.2 | MRC1 | RP13-147D17.3 | RP11-218E20.3 | PCDH10 | SCN7A |
| ANKS4B | TNN | PIEZO2 | MPDZ | RP11-471M2.3 | MBL2 |
| PCDHB3 | CNNM1 | LPA | GRK7 | ANKRD35 | DUSP27 |
| RP11-416I2.1 | RP11-1099M24.9 | RP11-728K20.2 | ABCG2 | AC005014.5 | SNX19P3 |
| CYP7A1 | RP5-1186N24.3 | CDCP1 | P2RY13 | CCR1 | AC092198.1 |
| TNPO1P2 | AGBL1 | PTPN5 | MAPK4 | RP11-555J4.4 | RP11-976B16.1 |
| RP11-805F19.3 | RP11-30P6.6 | TEDDM1 | SLC26A8 | RP11-641A6.5 | AL360294.1 |
| RPS21P4 | UBE3AP2 | SACS-AS1 | RP3-326I13.1 | RTP1 | ERVV-1 |
| AC104024.1 | RP11-1036E20.7 | RP11-497D6.5 | KCNIP3 | MRE11B | GAS7 |
| RP1-167G20.1 | RP13-870H17.3 | AC018742.1 | NTN4 | RP11-525E9.1 | ZNF33AP1 |
| NKAIN2 | AC121251.1 | CTTNBP2 | LINC00052 | FUT5 | TDRD9 |
| AC009313.2 | FIBIN | TSLP | SULF1 | CECR3 | GFRAL |
| RP11-325E14.2 | CTNNA2 | RP1-28C20.1 | RP3-347M6.2 | CTD-2383M3.1 | SLC17A8 |
| ENTHD1 | MMP2 | CAP2 | VSIG8 | MTND5P6 | ZNF501 |
| APOBEC4 | NPTX2 | SNRPFP1 | RP11-367H5.8 | CYP2G1P | CTD-2587H24.5 |
| CCT5P1 | LHFPL3 | PGLYRP2 | FAM149B1P1 | PRKACG | CCT6P2 |
| MATN3 | LL22NC03-84E4.8 | CTB-113P19.4 | RP1-269M15.3 | RP11-8H2.1 | SPTSSB |
| RP11-405A12.1 | bP-21201H5.2 | NMD3P1 | C11orf96 | REG4 | KCNG2 |
| TCAM1P | RGSL1 | EEF1A1P7 | TUBB8 | RP11-494M8.4 | RP11-513D5.5 |
| MKRN9P | FNDC4 | FKBP4P1 | UCA1 | HSPB2 | PSMC1P4 |
| C2orf27B | GNAI1 | RP11-403B2.10 | RP11-334E6.2 | FEV | KRT18P23 |
| KRT8P26 | ZSCAN5B | TMPOP1 | RP11-249L21.4 | AP000688.14 | ODF3L2 |
| TARDBPP2 | RP11-157G21.2 | IGFL4 | TMEM119 | FRMD6 | EIF4A2P4 |
| LINC00525 | POU4F3 | RP11-157F20.3 | C1QC | HNRNPA1P23 | ART1 |
| ACTBP7 | CHODL-AS1 | ST13P2 | AF131216.5 | PTCD2P2 | ACTG1P16 |
| OR2AE1 | USP12PX | RP5-1039K5.16 | OR10A5 | KRT18P33 | GTF3AP2 |
| PGGT1BP2 | LDHAP3 | KRTAP9-2 | PABPN1L | GYG1P3 | FAM170B-AS1 |
| KRTAP9-3 | SPDYC | DMRTC1B | IFNA5 | OR7E28P | RP5-907D15.4 |
| CT45A3 | HOMER2P1 | RP11-144G7.2 | RP11-1079K10.4 | NAT8 | NCR1 |
| SLC25A38P1 | CTD-2199O4.1 | OR10AB1P | MIR4500HG | RP11-1260E13.3 | KRTAP5-10 |
| SLC25A5P5 | CLDN10-AS1 | RP11-332G1.1 | RP11-286N22.16 | SDCBPP1 | RPL5P3 |
| OR7E31P | RP11-152F13.10 | RPL5P17 | RP11-415I12.3 | C19orf45 | MRGPRG |
| CDA | RP11-466C23.5 | AC009506.2 | AC068196.1 | RP11-18F14.4 | MIR99AHG |
| RN7SL812P | RP11-1112J20.2 | RP5-1039K5.17 | PPIEL | AP000318.2 | RP11-141P6.1 |
| AC084149.1 | RPS4XP13 | LINC00629 | RPS4XP7 | RP5-903G2.2 | CTD-2008P7.3 |
| PDZK1IP1 | RN7SL787P | RP11-344B2.2 | FRG1EP | RHOXF1 | AC084193.1 |
| RP11-287D1.4 | AC073869.1 | AC012493.1 | LINC01326 | NCAPGP2 | SNRK-AS1 |
| RP11-350E12.5 | RP4-816N1.7 | RP11-20I23.7 | SNX18P26 | MTND4LP14 | RP11-799B12.4 |
| HNRNPABP1 | TPM3P7 | SLC8A1-AS1 | RN7SL753P | RP11-798K23.5 | PGAM4P2 |
| RP1-230L10.1 | RP11-73M7.6 | RP11-440L14.3 | RP11-603B24.2 | AC009784.3 | VTI1BP1 |
| GYG1P1 | RP11-354P11.4 | CTD-3157E16.2 | RP11-436D10.3 | RP11-77K12.1 | XXbac- |
| | | | | | BPG252P9.10 |
| RP11-485M7.3 | TRPC4 | ARMC4P1 | LYPLA2P2 | RN7SL513P | RP11-553K23.2 |
| Metazoa_SRP | RN7SL411P | RP11-151G12.2 | AC008427.2 | RN7SKP130 | DPYD-AS1 |
| CTD-2196E14.8 | RP11-518L10.5 | RP4-655L22.4 | RP11-74E22.6 | RP11-3J1.1 | ERVH-1 |
| VTI1BP3 | C17orf98 | RP11-516C1.1 | ZNF197-AS1 | RP11-114F3.2 | RPS5P3 |
| RP11-100N21.1 | RASA2-IT1 | RP11-564A8.4 | CTD-2086L14.1 | RPS3P6 | NPM1P32 |
| RP11-477G18.2 | CYCSP11 | SUB1P3 | CTD-2288O8.1 | TLE1P1 | AC113607.3 |
| RP11-662B19.1 | RP1-292L20.2 | TCEB1P28 | RP11-573G6.9 | RPS9P1 | AC006116.24 |
| CCL19 | BNIP3P19 | CTD-2256P15.3 | RP11-77K12.4 | RP11-697H9.3 | CHCHD2P2 |
| RP11-708L7.6 | RP11-263K19.6 | RP11-698F20.3 | RPL9P33 | RP11-383C5.8 | RP11-177J6.1 |
| AJ239321.3 | RP11-455P21.3 | RN7SL154P | CTB-50E14.4 | RP11-566K11.7 | RP11-490E15.2 |
| RP11-930O11.3 | RP11-108B14.4 | RP11-394B2.6 | POLR2G | RP11-474B16.1 | RP11-413H22.2 |
| RP3-472M2.2 | LINC00400 | RNF138P1 | RP11-526K21.2 | PTMAP3 | RP11-770J1.3 |
| CTC-435M10.10 | RN7SKP203 | RP11-365D23.4 | RP11-13N13.5 | RP11-60I3.4 | RP11-543G18.1 |
| CTD-2568P8.1 | FAM184A | ARF4P4 | RP11-15A1.2 | AC005593.2 | TPT1P6 |
| Metazoa_SRP | RP11-15I11.3 | RP11-295M3.2 | RP4-680D5.8 | AC093106.5 | AC108463.2 |
| PIGUP1 | AC111200.7 | LLNLR-259H9.1 | DAZAP2P1 | RPS26P15 | RPS26P31 |
| H2AFB2 | IGKV1-33 | RPL12P30 | RP11-451N19.3 | RP11-568J23.4 | RP5-1092A11.2 |
| RP13-820C6.2 | RSL24D1P1 | RP11-774O3.2 | PLCXD2-AS1 | RP1-4G17.5 | IMMP1LP1 |
| RP11-719K4.3 | RP11-996F15.4 | RP11-3K24.1 | RP11-90E9.1 | RP11-73M7.9 | RP11-181K3.4 |
| RP5-1029F21.4 | OR7E62P | CTD-2529P6.4 | CTD-3065B20.2 | RP11-342M21.2 | AC005757.6 |
| RP11-54O7.11 | CTD-2182N23.1 | IGLV10-54 | ARHGAP22-IT1 | RPL23AP18 | RPL10P3 |
| RP5-1115A15.2 | CTC-209L16.1 | GS1-69O6.1 | RP11-665C16.8 | RPL34P31 | RP11-363I22.3 |
| CH507-39O4.2 | RP11-259O18.5 | RPL31P63 | IGKV1D-39 | RPL23AP88 | RP11-595B24.2 |
| H2AFZP4 | RP11-625L16.3 | RP4-771M4.3 | MAGOH3P | AC090952.5 | RP11-54O7.2 |
| RP11-74C13.4 | RP11-493L12.7 | RP1-41C23.4 | RP5-1087E8.3 | CTD-2566J3.2 | RP11-807G9.2 |
| RP11-517O13.1 | CTC-498J12.1 | C1orf189 | RP11-148O21.3 | RP11-253M7.4 | NUCB1-AS1 |
| RP11-571M6.18 | CTD-3105H18.9 | RP11-3L10.3 | RP11-791G15.2 | RP11-350D23.4 | RP1-102G20.2 |
| RP11-666A20.4 | RP11-188P17.2 | RP11-344P13.1 | RP11-713D19.1 | RP11-313P22.1 | AC093159.1 |
| RP11-532F6.4 | RP11-154D3.1 | CTD-2527I21.9 | RP11-483M24.2 | RP11-244B22.7 | RP11-230F18.6 |
| RP11-312B8.1 | RP4-585I14.3 | RP11-474G23.2 | PNPLA5 | RP5-836J3.1 | CTD-3193K9.3 |
| AC005616.1 | RP11-697E2.12 | PRUNE2 | CC2D1B | NDUFAF5 | BHLHE23 |
| GTPBP6 | GTPBP6 | POLR2D | SMYD2 | MMP1 | NR2F1 |
| MPPED1 | F7 | TBC1D22B | RP11-307C19.2 | KCNJ2-AS1 | ALS2CL |
| RP11-247L20.4 | DIO2 | RP11-359E7.3 | RP4-555D20.4 | RP11-1100L3.8 | RP11-158I9.8 |
| RP11-366M4.1 | COQ7 | CTC-428G20.1 | ABLIM3 | PLA2G5 | GCNT3 |
| INE2 | LINC00846 | RPSAP6 | ARHGEF5 | RP11-463O9.2 | RN7SL328P |
| GOLGA8F | C19orf33 | RP11-876N24.3 | RP4-633O19_A.1 | RP13-349O20.2 | FCN3 |
| RP11-745L13.2 | DNMT3B | RN7SKP163 | CCDC173 | ATP9A | RP11-10O17.3 |
| ATOX1 | AC139099.6 | NEMF | RP11-756P10.3 | LRRC41 | PLEKHG3 |
| CPSF4 | CCNB1IP1 | KCNH7 | AKR1C4 | RP11-259G18.3 | RP11-203M5.7 |
| NIF3L1 | AC078883.3 | RP11-363E6.3 | RP11-21L23.3 | ENOPH1 | RBPJ |
| DXO | MADCAM1 | MIAT_exon5_1 | NDUFC1 | RN7SL748P | SCML1 |
| PUM1 | BRD7P2 | TSSK6 | CHCHD10 | MNX1 | KRT8P27 |
| BTG2 | C3orf49 | RP11-302M6.5 | NUDT13 | CHRNA10 | ANKIB1 |
| RP11-548P2.2 | RP11-356K23.1 | GLYCTK-AS1 | CTD-2396E7.9 | FAM57B | CDH2 |
| RPL3L | TBCEL | GNAI3 | KIFC3 | CCDC65 | OGFOD1 |
| CSF3 | TP53BP1 | LINC00173 | PRICKLE4 | DCN | SAP18 |
| PROX1-AS1 | CWF19L1 | TMEM102 | FLCN | RP11-252E2.1 | LINC00578 |
| HMGB1P16 | JPX | MYO15B | KIAA0100 | CCDC144NL | BNIP3P16 |
| RP11-371A19.2 | NLGN2 | EEF1A1P6 | ZNF671 | NOX1 | LRRC37A5P |
| OR1M1 | CCDC126 | UTP14C | XXYLT1-AS2 | RALGAPB | THSD7A |
| RP11-3P22.2 | GRID2 | DPP9-AS1 | SHC1P2 | C1QTNF5 | ARL6IP6 |
| AGPS | BMS1P4 | SNORA23 | RP11-33N14.5 | AC008937.3 | RP11-622C24.2 |
| OSMR | RP11-568J23.8 | HIST1H2AM | ZNF7 | PKHD1L1 | LIN37 |
| BCYRN1 | AC009120.4 | SLC35B4 | ARF4-AS1 | SENP1 | RP5-890E16.2 |
| GPS2 | MTOR | RP11-49I11.1 | RBM4B | RP4-806M20.3 | SDE2 |
| RN7SL731P | PON3 | MRPL48 | CTA-25406.1 | RGAG1 | TSPYL2 |
| HMGB1P3 | LSM12 | TMEM232 | AC090587.5 | BCOR | C4BPB |
| FKBP4 | ERP27 | CPN1 | CTD-2369P2.4 | S100A11P1 | AC016738.4 |
| ZNF542P | CASR | KRT6B | HLA-DRB5 | FDX1 | DNA2 |
| HAPLN4 | RN7SKP176 | CTD-2287O16.4 | RP11-684B2.3 | Metazoa_SRP | FOXP4-AS1 |
| MYL5 | PFKFB3 | WNT10B | CHRNA3 | HSPA8P1 | UTS2R |
| ABCA3 | NAE1 | MAML1 | COMMD9 | AMACR | RP11-766H1.1 |
| RP13-539F13.2 | GNG12 | FANCD2 | RP11-266L9.8 | SNTG2 | CH507-154B10.1 |
| RP4-693M11.3 | HULC | RPL13P12 | CTC-429P9.4 | EIF3E | RP5-1021I20.5 |
| CTA-204B4.2 | SERBP1P1 | SPRR1B | ATG2B | UGGT1 | TUBA4B |
| CCNI2 | COX8A | RP11-359G22.2 | NFIX | RP11-708J19.1 | IL17RE |
| ASNSD1 | RP11-63M22.2 | ANP32B | RP11-434H6.7 | SEC23B | FMNL2 |
| JMJD4 | C1orf115 | LINC01170 | HSPB9 | IFNAR2 | RP11-701H24.9 |
| JOSD1 | GGNBP1 | DLGAP3 | THBS4 | LONRF3 | FUZ |
| bP-21201H5.1 | HPCA | RP11-7F17.8 | AP000254.8 | BCL2L12 | FAM21EP |
| ABCC6 | HIST1H4A | DUSP18 | TJAP1 | SPAG6 | RP11-486L19.2 |
| ACVR1C | RP11-90K6.1 | CHD8 | DNAJA1 | ARID3A | PRPF38A |
| TRAT1 | CTB-63M22.1 | TGFBI | APOA5 | CSNK2A2 | SLCO2B1 |
| VCAN | YME1L1 | AC011899.9 | RP11-388M20.6 | TRAPPC3L | LINC01465 |
| RPF1 | ZNF14 | TCF24 | CITED1 | ANAPC15 | FGF2 |
| HNRNPU | PUM2 | FLT4 | BDP1 | EXOC7 | PARP4P1 |
| UGT2B10 | STPG2 | DOK2 | CHMP1B2P | RNH1 | RP11-872J21.5 |
| FAM160A1 | IRF1 | TBX2 | RAD51AP1 | CTD-2302E22.4 | PDCL3 |
| HRG | OSBPL11 | RPS15 | AP4E1 | RP11-366L5.1 | HAP1 |
| CPB2 | INS-IGF2 | STK38 | NR2C2AP | COL6A1 | AC003002.6 |
| NF2 | PCP2 | CDKL2 | CCND3 | RP11-693J15.4 | ZBTB49 |
| CTRC | SLFN11 | RP11-9M16.2 | RP11-102L12.2 | UNC13A | ZNF410 |
| LINGO3 | TDP2 | CADM4 | ERCC6-PGBD3 | AC009961.3 | PZP |
| ATP6V1F | UXT | DTX2P1-UPK3BP1- | PIGR | FLYWCH2 | HTRA1 |
| | | PMS2P11 | | | |
| PPP2R5B | BTNL8 | RAB30 | INTS6 | ZNF91 | ADCK3 |
| SNORD3B-2 | LCN2 | CTD-3128G10.6 | C9orf16 | SMARCD2 | TAF4B |
| WHAMMP3 | EIF4G3 | USP31 | CDH12 | RIMKLB | PPP4R3B |
| LINC00963 | GAPDHS | HSPA8P15 | RP11-327F22.4 | USO1 | KANSL1-AS1 |
| EIF4A1 | MAP3K10 | AURKAPS1 | TCEA1P2 | ANKFN1 | RP11-203J24.8 |
| MRFAP1 | STAB1 | AFP | RP11-429J17.2 | YWHAG | ZC3H14 |
| NCBP1 | SPATA24 | RP11-115J16.2 | SDF4 | AC004980.10 | MASP1 |
| TFF3 | GPX2 | PRIM2 | SIX1 | OAZ2 | CCDC180 |
| RP11-529E10.6 | SGSM2 | RP11-269F19.2 | EZH1 | LINC00634 | JADE2 |
| SLIT3 | USP8 | GPN2 | TMEM42 | ADAMTS17 | ANXA1 |
| SLC46A2 | BACH1-AS1 | TMEM134 | RP11-69M1.6 | RPL5 | DCAF16 |
| YWHAB | PCBP1 | FBXO38 | C1orf100 | MIP | bP-2189O9.2 |
| TEX38 | POLD2 | ZNF548 | RGN | GARS | RPS16 |
| NOL12 | MORC2-AS1 | METAP2 | STK11 | RP11-316M1.12 | RP11-47I22.2 |
| TCN1 | LSM5 | AAMDC | TNFSF14 | RP3-475N16.1 | DEK |
| ECI1 | ZNF154 | MED30 | MCM8 | ZNF251 | C9orf172 |
| CUTA | SECISBP2L | KB-1460A1.5 | HMGA2 | TLDC2 | RP5-997D16.2 |
| TRAF3IP1 | C1orf52 | CACNA1F | RPSAP9 | NPIPA2 | ETNPPL |
| SYNJ1 | RP11-74E24.2 | CTB-113P19.5 | HAUS8 | PSMC5 | CIART |
| TRIM60P14 | IGHV5-78 | DAZAP2 | MPC1 | RP11-643G16.4 | AP1M2 |
| RP11-338K17.10 | FAM153A | ZCCHC9 | GNA13 | STK17B | TDO2 |
| KLHL9 | NADSYN1 | ARL5C | CYP2C9 | LGALS9C | CTBP2 |
| CASC11 | SPAST | SPDL1 | PIGA | AMOTL1 | ZNF292 |
| COX7A1 | MPV17 | CLIP2 | YY2 | ADCK5 | C6orf25 |
| AC093901.1 | PCNA | SLC25A46 | ADAMTS7P1 | SNX13 | ADGRA3 |
| TCTE3 | AC017116.11 | RP11-20I23.2 | PLEKHB1 | RPL38 | PSMA4 |
| KBTBD8 | LINC01344 | RP11-290L1.4 | YARS | OGT | IL9R |
| IL9R | SRP9 | XAB2 | PLEKHJ1 | CTD-2547L24.3 | ECT2 |
| ELOVL3 | HBP1 | RP1-257I20.14 | TBC1D14 | RSPH10B2 | CLNS1A |
| MICB | MTMR9 | IL13RA1 | PAXIP1 | C6 | SRFBP1 |
| EAPP | UTP23 | MTO1 | RP11-521B24.5 | SH2D6 | SLC2A10 |
| CNTD2 | RP11-646I6.5 | RP5-1057120.4 | SMURF1 | MAGEA2B | F9 |
| ZNF326 | PAICS | RNA5-8S5 | RNA5-8S5 | RNA5-8S5 | RNA5-8S5 |
| RNA5-8S5 | RNA5-8S5 | RNA5-8S5 | RNA5-8S5 | C16orf59 | SMPD4 |
| GAREML | CETN3 | IGF2R | ADAMTS1 | CROCCP3 | TM2D3 |
| LRRC16B | MT1E | RRAGC | MBTPS1 | SMG1P7 | RN7SL4P |
| RP11-160C18.2 | SBNO1 | HSPA13 | SYNJ2 | CTC-458I2.2 | CD70 |
| PRB2 | NENF | AGAP4 | ADD2 | UQCR11 | ARIH1 |
| ELMO1 | DNAJC9-AS1 | ATP6V1B2 | EPHA2 | PRDM1 | MARVELD1 |
| HP | C4orf27 | RP11-148K1.12 | LINC01447 | ZNF684 | GNL2 |
| COX7B | SSB | IDH1 | RAP2B | TMEM41A | CYFIP1 |
| HNRNPLP2 | SCAP | ADH1B | CLEC18C | PPP1R13L | RSU1 |
| KRT6A | ADAM17 | GTSE1-AS1 | ADAT3 | RNF185 | RP11-478J18.2 |
| SNRPC | AC006011.4 | BORCS6 | RP11-320N21.2 | APLP2 | NCAPH2 |
| LINC00899 | CTD-2201E18.3 | EXOSC3 | ELAVL1 | UPF2 | LINC01249 |
| RP11-2E11.6 | B3GALT5 | ADCY10P1 | ZC3H11A | SMIM2-AS1 | NIPBL |
| PARP6 | MKNK2 | AC107081.5 | KMT2C | TTF1 | DKC1 |
| PUF60 | PTGES2 | N4BP2 | RP11-333E13.4 | ARSD | ZNF263 |
| SHARPIN | FAR2 | MAP1B | SLC7A11-AS1 | DDX1 | REV3L |
| TMEM187 | SSU72 | NYX | TTBK2 | FAIM2 | FAM66C |
| ITGA5 | COX6B2 | LINC01624 | RP4-728D4.2 | RP11-443B7.1 | CTD-2540F13.2 |
| RP11-15L13.4 | ATPAF2 | RP11-87H9.4 | CTD-3064H18.4 | GGNBP2 | EIF2S3L |
| GTPBP2 | SPEG | EPG5 | GPHN | FGF14-AS2 | COPS3 |
| CCDC94 | TCP1 | HERC2P9 | DCPS | TRABD2A | GPR22 |
| SETD2 | JAGN1 | PRDM4 | RP11-135F9.4 | OXSR1 | SRSF7 |
| DCST2 | SERP1 | QSER1 | PLEKHH3 | C18orf54 | GCC2 |
| CNTRL | COL4A2 | TNNI3 | NME2 | ZC3H18 | SNRNP70 |
| RP11-134L10.1 | MRPL36 | KNSTRN | COLGALT1 | PSD4 | SAMD9 |
| SLC10A3 | CNN2 | ADCY5 | GS1-57L11.1 | COX15 | RP11-113D6.10 |
| FOSB | ACD | AC074212.6 | CHMP3 | PON1 | WDR74 |
| WDR3 | TMEM132B | ACSL6 | POLR2K | AC010761.8 | ARL17B |
| IGKC | BRWD1-AS2 | VPS16 | CEP78 | GABRE | MCF2 |
| TLR10 | TRAPPC12 | CCRL2 | STAU1 | TMEM125 | C2orf69 |
| RP11-496H1.2 | TSFM | SERPINA9 | RP11-508N22.12 | SNRPD2 | SLC26A4-AS1 |
| RP4-548D19.3 | SGTB | RP3-477O4.14 | SBK1 | CEP164P1 | ZNF606 |
| ASTE1 | ESR1 | EIF3J | EPN1 | TFPI | HBS1L |
| HIST1H2AE | CERS5 | B3GNT9 | AC006077.3 | ATRIP | ST7-OT4 |
| TICAM1 | DPY19L2 | BMS1P1 | DDX17 | PIN1 | ARMT1 |
| NARS2 | RPP21 | DDX31 | NRROS | CFTR | MYH16 |
| HS3ST1 | AOC1 | HECW1 | KLHL13 | CYP26B1 | HSPB6 |
| PDK4 | SLC4A1 | PRSS22 | ITGA2B | ASB4 | TMEM98 |
| CX3CL1 | ETV1 | SCIN | PROM1 | NOS2 | DNAH9 |
| MATK | TKTL1 | SELE | FMO3 | SYN1 | CRY1 |
| PGLYRP1 | MLXIPL | ETV7 | SLC6A13 | PRSS3 | ANOS1 |
| NR1H4 | SLC7A14 | MAMLD1 | DPEP1 | NPC1L1 | STMN4 |
| IL20RA | CLCA4 | IGF1 | VSIG2 | MARCO | CYP24A1 |
| ADGRA2 | SLC7A9 | NRXN3 | EHD2 | INSRR | APBA2 |
| EFCAB1 | OTC | SLC18A1 | HOXC8 | RAI14 | SOX30 |
| RAB27B | MYO16 | FAM65C | USH2A | PREX2 | ANO2 |
| CC2D2A | LMO3 | KITLG | TNFRSF9 | EPN3 | COL23A1 |
| FSTL4 | METTL24 | KCNQ1 | ENTPD2 | CHRDL2 | DCBLD2 |
| SERPINB3 | TMCC3 | TBXAS1 | GNA15 | CREB3L3 | RIMBP2 |
| LZTS1 | PRDM6 | WNT8A | CDH3 | LIMCH1 | TSPAN32 |
| WISP2 | ST3GAL6 | POU1F1 | ROPN1 | TLE2 | ME1 |
| TIE1 | SLC9A3 | NGEF | MPPED2 | FGFR2 | EML1 |
| PHKA1 | PDZD4 | ATP2B3 | INPP5A | STON1-GTF2A1L | ADGRF5 |
| MAOB | DRD4 | HES2 | PTPN3 | ST6GALNAC1 | CNGB1 |
| CHAT | CAMK2A | MS4A12 | LMCD1 | CDH19 | SPP2 |
| RHOBTB1 | PVR | CYP2W1 | PTGS2 | PPP2R2C | CA12 |
| NUAK1 | ARHGEF10L | KCNQ2 | SEMA3C | CACNG5 | CACNG4 |
| PLD1 | ATP12A | PAX2 | RGS11 | PLXNA2 | ACTL6B |
| PAK3 | DCX | ACTN2 | FKBP6 | CST7 | AMPH |
| FAP | HOXA9 | FGF20 | BRINP1 | CDH17 | OSBPL6 |
| DUSP13 | DDX43 | SCTR | COL5A3 | KCNN2 | CXCL2 |
| CACNA1S | PKP1 | LRP2 | SLC13A1 | CADPS2 | PCDHB4 |
| PCDHA6 | MPP4 | PGR | OPRK1 | SEMA5B | TRPM3 |
| GRHL2 | SLCO1A2 | COL16A1 | GCKR | MECOM | IGSF9 |
| WNT11 | FOLH1 | EPDR1 | HSD17B2 | ACHE | TMPRSS11E |
| CETP | MT3 | LPCAT2 | TFAP2C | SULT2B1 | SLC15A1 |
| TMEM40 | ARHGAP28 | SMOX | SIGLEC1 | C20orf194 | CPXM1 |
| F11 | CFAP61 | RPH3A | NOS1 | HEPH | FETUB |
| CD209 | NRCAM | SLC26A3 | IL5RA | CMA1 | DAZL |
| FMO2 | TDRD1 | CRTAC1 | TREM2 | PGC | NCR2 |
| NRP1 | IGFALS | MISP | P2RX6 | SLC7A4 | SEC14L3 |
| PRODH | PLA2G3 | LGALS2 | SEZ6L | RP1-37E16.12 | SLC16A8 |
| SLC5A1 | TPTEP1 | TIMP3 | APOBEC3H | PDGFB | ACR |
| PNPLA3 | UPK3A | IL2RB | CHADL | KCNK10 | GZMB |
| C14orf105 | PLEK2 | RIN3 | LGMN | CHGA | SLC8A3 |
| BDKRB1 | MMP9 | VSX1 | PROCR | HNF4A | SALL4 |
| DOK5 | HRH3 | BIRC7 | COL20A1 | ISM1 | SLC52A3 |
| ANGPT4 | RSPO4 | PROKR2 | SIRPB1 | FERMT1 | PDYN |
| CCM2L | PLCB4 | ASIP | CST4 | EPPIN | CELF4 |
| CDH20 | LAMA1 | VSIG1 | GUCY2F | ATP1B4 | CHRDL1 |
| PAGE4 | GLRA2 | BMX | LUZP4 | SMARCA1 | ASB9 |
| PPP1R2P9 | GATA1 | GPR50 | CD40LG | KLHL4 | SRPX2 |
| SYTL4 | ZDHHC15 | NALCN | HTR2A | MLNR | SGCG |
| IRG1 | MEDAG | OLFM4 | MSLN | CCL22 | CCL17 |
| NECAB2 | WFDC1 | FOXF1 | AQP8 | CPPED1 | SALL1 |
| AQP9 | RASL12 | GOLGA8UP | CD276 | TGM5 | PDGFRL |
| CA2 | TRPA1 | SFRP1 | JPH1 | DKK4 | SH2D4A |
| TUSC3 | ADAM2 | TULP2 | CGB | KCNA7 | DKKL1 |
| CLEC4M | NOVA2 | KIR3DX1 | LILRB1 | LILRA1 | OLFM2 |
| SYDE1 | CASP14 | EBI3 | SHD | MYH14 | CD33 |
| CEACAM5 | CRX | SYNGR4 | CABP5 | LILRB5 | SLC5A5 |
| ATP4A | ATP6V0A4 | TFEC | CAV2 | HOXA2 | VIPR2 |
| EVX1 | CHN2 | GRB10 | COBL | PAX4 | MOGAT3 |
| NOBOX | MYL10 | MEOX2 | GLI3 | PRKAG2 | GALNTL5 |
| LHX2 | SH3GL2 | PTGDS | GATA3 | PKD2L1 | PLEKHA1 |
| UNC5B | TLX1 | FGF8 | NEURL1 | SORCS1 | CYP2C18 |
| KRT23 | CRYBA1 | ASPA | HOXB6 | SGCA | ABCC3 |
| CACNG1 | PMP22 | SULT1E1 | ODAM | NMU | UCP1 |
| TBC1D9 | WFS1 | ANXA10 | SOD3 | CPZ | SLC2A9 |
| CLNK | HGFAC | FAM149A | B3GAT1 | P2RX3 | MS4A6A |
| MS4A4A | CCKBR | FOLR1 | FOLR3 | APOA4 | UPK2 |
| ELMOD1 | CALCA | VWF | DAO | SLC6A12 | PRMT8 |
| CUX2 | GSG1 | RERGL | IL26 | IFNG | SLCO1B3 |
| APOBEC1 | NANOG | ST8SIA1 | RFX4 | SASH1 | UST |
| OPRM1 | TULP1 | IL17F | MDGA1 | RBM24 | TSPO2 |
| SIM1 | COL9A1 | SLC17A2 | ADGRG6 | PDE10A | MDFI |
| SMOC2 | PRPH2 | FAM46A | CLIC5 | TBX18 | NUDT12 |
| SEMA5A | C7 | GHR | NME5 | PCDHB5 | PCDHB6 |
| HAVCR1 | IL12B | NPR3 | AGXT2 | PRLR | SLC12A7 |
| IL4 | LIFR | DPYSL3 | PDGFRB | HRH2 | UNC5A |
| CLDN16 | AMOTL2 | RBP2 | RBP1 | BCHE | FGF12 |
| GNAT1 | C3orf14 | UPK1B | SLC4A3 | ADAM23 | CCL20 |
| OTOF | GRB14 | CLIP4 | TLX2 | EVA1A | EFEMP1 |
| IGFBP5 | EFHD1 | IL1R2 | PRKAG3 | SMYD1 | IL1R1 |
| IL18R1 | IL18RAP | SLC9A2 | MLPH | RND3 | KISS1R |
| VAX2 | ATP6V1B1 | EPHA4 | PARD3B | PRRX1 | MARK1 |
| TNR | NPHS2 | PDC | WLS | RPE65 | CD2 |
| NID1 | NT5C1A | SIPA1L2 | ST6GALNAC5 | ADGRL2 | MFAP2 |
| RGS4 | PLA2G2D | CR2 | MPL | ARTN | CNN3 |
| F3 | FASLG | A4GNT | MMP8 | ZNF541 | TNNT2 |
| CTGF | STBD1 | PCDH17 | CCND2 | FGF23 | NR4A3 |
| CSF3R | ONECUT2 | ESRRB | CNRIP1 | PPP1R3C | PYROXD2 |
| PRLHR | SFRP5 | CRHR1 | TEK | INSL4 | CCDC170 |
| PCDHB8 | TNFSF18 | TTLL2 | KCNJ5 | KIAA1217 | PLXDC2 |
| IQSEC3 | ENOX1 | TNFSF11 | DUSP4 | CHRNA2 | ADRA1A |
| NPPB | TBX4 | LRAT | TAS2R8 | KCNJ8 | TAS2R9 |
| GJA3 | HCRTR1 | CCR2 | CPXM2 | TMIGD3 | SV2C |
| MYOG | PRM2 | OCM | KIAA0087 | NPY | INHBA |
| ARL4A | PHF24 | CNTFR | AKR1D1 | SFTPA1 | CHST3 |
| BICC1 | ARL4AP1 | SSPN | CCDC70 | GUCY1B2 | HOXC13 |
| HOXC11 | NFE2 | DBH | IL13RA2 | PLP1 | ATXN3L |
| G0S2 | DAW1 | INHA | OBSL1 | FAM124B | PI3 |
| ARHGAP40 | MATN4 | GDAP1L1 | EDN3 | RBPJL | ZBP1 |
| MAGEA10 | IQSEC2 | RP11-663P9.2 | IRGC | GRM4 | BTN2A3P |
| MOCS1 | SPDEF | TREM1 | ATXN1 | OPN5 | AC105760.3 |
| EREG | DKFZP434K028 | SCGB1D2 | WNT1 | SLC10A2 | BMP4 |
| TEKT3 | BARHL1 | SRMS | LINC00029 | NPBWR2 | C20orf195 |
| IL37 | GRIA3 | FOXA2 | FAM182A | BMP2 | LRRN4 |
| OTOR | DEFB129 | GDF5 | BPIFB9P | AMOT | SLURP1 |
| GPR42 | PRKCG | PRDM7 | NXF5 | OMD | EDN2 |
| BCL11B | IL22 | TSPAN8 | CRYGN | TRPV5 | ADGRE2 |
| IL17B | TNFRSF19 | KDR | GAL3ST1 | RFPL1 | RFPL3 |
| CDC42EP1 | MCHR1 | GALR3 | LIF | KRT17 | RNF112 |
| CPA4 | FOXP2 | HOXD10 | HOXD11 | ISLR | LOXL1 |
| KCNC1 | FAM64A | SHBG | LRRC6 | MTUS1 | SIGLEC9 |
| KLK10 | EGLN3 | FOXJ1 | CDH15 | SHC2 | PLPPR3 |
| NXNL2 | STARD8 | TSPAN16 | C19orf80 | CNN1 | ZSCAN10 |
| PVRL2 | GADD45G | LRCH2 | GDF1 | MAS1 | PXDN |
| GDF15 | HRC | OR11H1 | OLFM1 | SAG | TNNT3 |
| CALY | RBBP8NL | FIBCD1 | PNCK | DUSP9 | LRP3 |
| PRRG1 | BPIFA2 | COX4I2 | C1QL1 | GFAP | PYY |
| CCL25 | LRRC4B | GFPT2 | BARX1 | CA6 | RHOXF2 |
| FSHB | LIN28A | LRRC9 | PPARG | HHLA1 | RAMP1 |
| RGS22 | BTBD3 | RHBG | ATP1A4 | NES | VSTM2L |
| KANK4 | ATP8A2 | TPTE2 | GPR12 | CHRM3 | LGR6 |
| DCLK1 | SPG20 | RNF128 | PRAM1 | LGALS12 | GSTT2B |
| MYO18B | FAM83F | SEC14L4 | ZDHHC8P1 | SFTPD | TMTC1 |
| LYVE1 | DUSP26 | GSTM5 | GSTM3 | CHIA | PTGFRN |
| ADAM30 | CD101 | VTCN1 | ANO3 | CFHR4 | CRB1 |
| RERG | SLCO1B1 | PRH2 | USP26 | FAM127A | PIWIL4 |
| MTNRIB | HOOK1 | CYP2J2 | DSC2 | DTNA | FHOD3 |
| GIF | APLNR | PDGFRA | CLDN10 | DZIP1 | KDELC1 |
| ADAMTS8 | FAM189A2 | CCNJL | HNF1A | TBX3 | CD36 |
| UGT2A3 | FAM71F1 | ADGRB3 | NT5E | MRAP2 | LCA5 |
| ELF5 | PRRG4 | AMHR2 | GLS2 | NMBR | EGR4 |
| DYSF | CHST5 | KCNK1 | CAPN9 | LECT1 | SCEL |
| EDNRB | IL6 | DGKB | MYCBPAP | SCN2A | GALNT5 |
| GATA4 | HLX | IL36G | IL36A | GAD2 | KLF4 |
| ZFP37 | WDR38 | OR1L4 | ENPP2 | IL33 | CCL21 |
| CAPN11 | PI15 | TTPA | TRPC6 | MMP20 | C11orf70 |
| BTG4 | POU2F3 | FXYD6 | FXYD2 | MMP13 | UNC13C |
| UACA | STRA6 | ARHGAP29 | CLCA2 | DNASE2B | CYP1B1 |
| CYP2C8 | CH25H | BTBD16 | TACC2 | PLA2G12B | OIT3 |
| ADAMTS14 | STAT4 | MSTN | SLC40A1 | CILP | HCN4 |
| HERC5 | PRKG2 | GPAT3 | BMPR1B | TRPC3 | FRAS1 |
| SHROOM3 | C4orf17 | PDE6H | CPNE8 | SLCO1C1 | COL2A1 |
| ANP32D | LRIG3 | TPH2 | LGR5 | PTPRQ | LUM |
| SDSL | FOXN4 | DHH | ACVRL1 | METTL21C | RAB20 |
| SSTR1 | RTN1 | RDH12 | SLC25A47 | DUOXA1 | DUOXA2 |
| DUOX2 | HDC | ARRDC4 | CYP11A1 | GOLGA6D | RLBP1 |
| WDR93 | NTRK3 | ITGAX | CHST4 | CLEC18B | ADAMTS18 |
| CDH11 | CDRT15P1 | ZNF287 | DPEP3 | TOM1L1 | SKAP1 |
| ABCA8 | C17orf64 | ASXL3 | SLC25A52 | ASGR1 | PIK3R5 |
| CARD14 | SECTM1 | RNF165 | CBLN2 | STAC2 | CIB3 |
| HUNK | COL6A2 | IL19 | CBLC | ADAMTS10 | SLC6A3 |
| FBN3 | TM4SF5 | GPR32 | ACPT | KLK3 | CTD-2545M3.6 |
| IGLON5 | MMEL1 | CFAP74 | PRDM16 | PADI3 | FHAD1 |
| PADI1 | MYOM3 | DMRTA2 | HSPG2 | TINAGL1 | AZIN2 |
| SLC44A3 | IGSF3 | ZNF697 | FNDC7 | PROK1 | XCL1 |
| MAEL | DPT | F13B | LHX9 | RORC | SELENBP1 |
| FLG2 | S100A8 | PKLR | FLG | LEFTY2 | SOX13 |
| ETNK2 | SYT2 | ABCG8 | TRIM43 | MALL | THNSL2 |
| THSD7B | GULP1 | PTH2R | ABCA12 | TRPM8 | GRIP2 |
| CNTN4 | GADL1 | POMGNT2 | ACKR2 | FAM198A | ITGA9 |
| CTDSPL | COL8A1 | ADGRG7 | TAGLN3 | IGSF11 | BOC |
| AGTR1 | UCN2 | SLC10A4 | SLC10A6 | SNCA | FABP2 |
| SFRP2 | RNF175 | PDGFC | ROPN1L | ADAMTS16 | MYO10 |
| UGT3A1 | GZMA | MEGF10 | ADAMTS19 | CXCL14 | LECT2 |
| TIMD4 | SPINK7 | CPLX2 | TENM2 | KCNMB1 | DCDC2 |
| SLC17A4 | KAAG1 | PLA2G7 | DAAM2 | MLIP | HMGCLL1 |
| LGSN | TPBG | GABRR1 | CLVS2 | TAAR6 | TAAR8 |
| TAAR1 | VIP | SLC22A3 | EGFR | IGFBP1 | ASB15 |
| ZAN | STRA8 | EPHA1 | NLGN4X | SHROOM2 | TMEM47 |
| AKAP4 | CXorf36 | AWAT2 | CPXCR1 | NXF3 | FRMPD3 |
| IGSF1 | GPC3 | GPR119 | CSGALNACT1 | DOCK5 | ST18 |
| TRIM55 | SLC26A7 | RSPO2 | MAL2 | IFNA16 | IFNK |
| NTRK2 | SHC3 | OR13C4 | ZNF462 | ACTL7B | HMCN2 |
| IDI2 | LCN9 | CACNA1B | FAM171A1 | PARD3 | ANKRD30A |
| A1CF | CDHR1 | ANKRD1 | HTR7 | NPFFR1 | CYP17A1 |
| INA | ADAM12 | SYT8 | PAMR1 | KLHL35 | TENM4 |
| NCAM1 | DRD2 | GLB1L2 | GRIK4 | SLC22A8 | ZP1 |
| PLCH2 | KIRREL3 | SCN2B | JPH2 | DUSP15 | CDH22 |
| LINC00266-1 | SLC22A9 | MMP3 | CNTN5 | KLRF1 | MKX |
| FXYD4 | FCGR1A | LATS2 | MIA2 | HNMT | ADRA2A |
| VEGFC | CCDC83 | PRSS23 | PPP1R1C | DOCK1 | IL18 |
| TKTL2 | ENKUR | THRB | DLG5 | MAGI1 | ME3 |
| ADAMTS12 | CCDC3 | POU4F2 | EDNRA | AKR1C6P | WWC2 |
| SLC35F4 | GFRA1 | GLT1D1 | RBM46 | ZNF37CP | KCNE4 |
| TUBA3E | HSPB8 | CYSLTR2 | PTH | GUCY1A2 | BOLL |
| ZNF256 | OLAH | CCDC50 | PLEKHH2 | GRIA4 | IGSF10 |
| GJA1 | DDX4 | CLEC4F | TCTEX1D1 | SLC16A12 | PTPRK |
| CNTNAP4 | RAB3C | PLOD2 | ZIC1 | BMP6 | PLA2R1 |
| ADGRF1 | ADGRF4 | FRMD1 | FAM81B | LURAP1L | CNKSR3 |
| TGIF2LX | TMPRSS11D | PID1 | KCTD15 | CLCA3P | HS3ST3A1 |
| SEMA3D | ANKRD29 | THY1 | PANX3 | GPR15 | CERS3 |
| ABCA9 | ABCA10 | TNIK | WNT3A | PPP1R3A | GBP5 |
| MMP21 | CNTNAP3B | PDLIM3 | WNT7A | EPHB1 | Sep-14 |
| UNC93B2 | ODF1 | VSIG4 | ALS2CR11 | SPAG17 | SAXO1 |
| SLC24A2 | RAET1L | NAT2 | MAGEA8 | GNA14 | GNAQ |
| WIF1 | UGT2B4 | KCNMA1 | ALX3 | CFAP161 | ADAMTSL3 |
| ART3 | SLC28A1 | CXCL13 | NAA11 | FUT6 | KCNS2 |
| LHFPL4 | MPV17L | SHCBP1L | SLC6A1 | SYN2 | CDCP2 |
| STEAP2 | HTR5A | IL34 | CACNA1D | ARSE | KIT |
| CACNA2D3 | MUM1L1 | KCNJ6 | ERG | SVOPL | SLC34A2 |
| ACAN | DPYSL5 | AGAP1 | SLC30A2 | TRIM63 | GRHL3 |
| GALNT14 | XDH | DZIP1L | MRAS | ESYT3 | CLSTN2 |
| COLEC12 | SLC13A3 | SHROOM4 | KCNB1 | CPA2 | CPA5 |
| PPP1R9A | POM121L2 | SLAMF8 | DUSP23 | HTR6 | ITLN2 |
| NPM2 | FGF17 | DMTN | WNT9B | CACHD1 | STC1 |
| TNNI1 | CBR3 | GJD2 | CLDN14 | GOLGA6A | SCUBE1 |
| PADI4 | CES5A | CELF3 | TCHH | ADGRG5 | DRC7 |
| LRRC36 | ZDHHC1 | FAM131B | LYPD5 | PTGIR | KALRN |
| TMPRSS3 | UBASH3A | CRYAA | LRRC3 | ZNF208 | LCN1 |
| GPSM1 | HIPK4 | TMEM190 | PLPP7 | CCR5 | PTH1R |
| HK3 | COL26A1 | DMKN | NPHS1 | HCRT | SIGLEC11 |
| MPP3 | IP6K3 | TREML1 | BCL6B | PRR35 | MEIOB |
| ASRGL1 | TAL1 | BEND5 | ACOT11 | LEXM | BSND |
| PLPP3 | PRKAA2 | SLC45A1 | KNCN | TMEM82 | SYNC |
| ALPL | TTLL10 | MEGF6 | CCDC27 | LRRC53 | VCAM1 |
| NLRP3 | KCNJ9 | OLFML2B | FCGR3B | FAM71A | TDRD5 |
| WDR64 | PKDCC | IL20 | C1orf145 | CAPN13 | KCNF1 |
| KCNJ3 | SPATA18 | CFAP221 | INHBB | HPGDS | SMCP |
| IVL | SPRR3 | BMP10 | PGLYRP4 | S100A9 | TDRD10 |
| GABRB1 | C1orf106 | LINC01116 | SLC22A15 | IGFN1 | PROK2 |
| LMOD1 | ADORA1 | FBLN2 | SPTA1 | FAM86KP | WDFY3 |
| PRICKLE2 | ADAMTS9 | DCLK3 | DNASE1L3 | PCOLCE2 | CXCL5 |
| TM4SF18 | TCF23 | DNAJC5G | CLEC3B | SLC6A20 | RTP3 |
| LRRC2 | NMNAT3 | GRIK3 | CFAP100 | RHO | IGF2BP2-AS1 |
| CLDN19 | HAND2 | MAP9 | ASB5 | C4orf45 | TMEM144 |
| NPY1R | SLC45A2 | ZNF474 | F2RL1 | HTR4 | ESM1 |
| CDC20B | ENPP6 | FAM170A | KLKB1 | FOXQ1 | ADGRF2 |
| GRIK2 | SAMD3 | DACT2 | FAM183BP | MYOZ3 | GPR85 |
| KIF6 | INTS4P1 | ZNF704 | CHMP4C | PGAM2 | DLC1 |
| HNF4G | TNFRSF11B | CA3 | ALDH7A1 | BAALC | FREM1 |
| TMEM71 | MAMDC2 | PRSS37 | CPA6 | RASEF | TRPV6 |
| C7orf34 | TMEM246 | WBSCR27 | C9orf84 | PTCHD1 | OR1Q1 |
| OR1K1 | CLDN3 | NLGN4Y | AQP7 | SLITRK5 | ARMC3 |
| FAT3 | CCDC67 | GPR101 | CLDN2 | LRFN5 | PHOX2A |
| PKNOX2 | NGB | FAAH2 | DACT1 | BEND7 | SLC18A2 |
| TMEM52B | FRMD7 | RET | RNASE7 | OTOGL | SERPINA12 |
| PDZRN4 | SLC6A5 | PASD1 | RP11-299H22.3 | ADAMTS15 | SVOP |
| LRTM2 | LARP6 | ASPG | ALKBH3 | GABRB3 | SCN3B |
| TMEM100 | MOGAT2 | SERPINB7 | OR5P1P | WDR72 | PLD4 |
| TMEM130 | FAM86GP | CLEC3A | BEAN1 | SLC38A8 | CPLX4 |
| RRAD | SERPINB12 | ACSM1 | STRCP1 | LDHD | TAC3 |
| GREM1 | NYAP1 | MS4A8 | CCDC178 | MS4A15 | SGSM1 |
| B4GALNT2 | SUN5 | SAMD14 | TMEM92 | LINC00483 | C16orf92 |
| GOLGA6C | NOD2 | LOXHD1 | RBFOX3 | CD3D | OR51I1 |
| GNG8 | LPO | CA4 | LINC00905 | ZNF528 | AQP2 |
| CDC42EP5 | LAIR2 | PSCA | ATCAY | TMIGD2 | KLK6 |
| KLK11 | KRT80 | ACER1 | CD300C | TEKT1 | PRR25 |
| CASKIN1 | RAB3IL1 | SLC22A11 | SCARA3 | SCARA5 | ZNF355P |
| OR2B2 | KCNJ4 | HIST3H3 | PTGDR | FOXI1 | ACOX2 |
| MOBP | C8orf22 | XIRP1 | LINC00917 | BDKRB2 | SFTPC |
| PHYHIP | HFE2 | CHRM1 | ADAM18 | GDNF | FAM84B |
| KCTD19 | CA7 | OR13J1 | CTRB1 | CTRB2 | STXBP6 |
| PXDC1 | ROR2 | RP3-509L4.3 | AR | C8orf46 | FAM110B |
| ARMC4 | PCSK9 | CXCL10 | SH3TC2 | RP11-220D10.1 | SHE |
| P2RY12 | RNASE6 | NPR1 | COL22A1 | CCDC8 | CT55 |
| GJB1 | CLIC3 | BNC1 | ANTXR1 | PROKR1 | LDB2 |
| NLGN1 | LINGO1 | RBMY1HP | CTNND2 | MUC3A | TM4SF4 |
| FRMPD4 | MYO7B | MYRIP | HMP19 | RNF150 | CCDC144A |
| CR848007.2 | ANKK1 | CNGB3 | CST1 | SEMA3E | DCLK2 |
| KRT78 | FAM71B | SLC26A5 | SGCD | OR5E1P | TTLL6 |
| SYT9 | GPR37 | OR10A4 | SDR16C5 | OR10A2 | OR2AG1 |
| LMOD2 | CELP | CYTL1 | OSCAR | OR7G2 | PKHD1 |
| MBD3L1 | PRKCDBP | CEACAM3 | DDI1 | LINC00208 | FPR2 |
| FPR1 | ALK | GIMAP8 | RLN3 | PROL1 | SMR3B |
| TMEM37 | SOSTDC1 | KRT15 | KRTAP4-4 | PDE7B | KRT27 |
| OR1L3 | SSX6 | OR1B1 | SPACA5 | COL24A1 | RXFP1 |
| NEUROD2 | MAP6 | PTCRA | P2RY6 | LKAAEAR1 | C1orf111 |
| VAT1L | KNDC1 | ANGPTL7 | PRND | AQP4 | FBXW10 |
| CYP4F22 | FOXB1 | ZNF556 | MAL | EPHX4 | LRRC15 |
| CD8B | CALB2 | MBOAT1 | AZU1 | TPSAB1 | CLEC7A |
| NEGR1 | CERS6 | SPTLC3 | ABCG4 | OR5B2 | OR9I1 |
| INSL5 | EFCAB3 | TTC36 | PRSS30P | FUT9 | C2orf54 |
| OR10H5 | CTSW | SMPDL3A | OR10AD1 | THEMIS | MOS |
| HOXC5 | OVOL1 | FLJ42102 | MYEOV | XKR3 | GXYLT2 |
| HPSE2 | BNC2 | RXFP4 | CCDC63 | ACSM6 | RHOD |
| CYSLTR1 | MAB21L3 | VANGL1 | TERF1P1 | DMRT2 | SLC2A14 |
| OR10K1 | NDNF | IQCF1 | OLR1 | GLIPR1L1 | DAB1 |
| CCDC36 | PTPRM | NLRP13 | XCR1 | SULT1B1 | HSPB7 |
| CD7 | MARCH10 | RP11-89N17.1 | C1QTNF1 | SNURFL | RP11-18M17.1 |
| RAX2 | LRRC63 | CD34 | MSRB3 | NWD2 | SNX31 |
| ADCY6 | SLC16A11 | GLIS1 | PODN | SLC6A19 | ABRA |
| VWC2L | GALNTL6 | LINGO2 | IGDCC3 | SLC26A9 | TTC9B |
| KLK15 | GOLT1A | NPAS4 | CMKLR1 | KY | BRSK2 |
| FAM47DP | LEP | RP11-79L9.2 | WDR49 | BTC | CATSPERD |
| VN2R1P | SEZ6L2 | P2RY14 | GPR171 | CD164L2 | CA5A |
| ZG16 | CHST2 | WFDC5 | TMEM51-AS1 | ABO | GAL3ST3 |
| CHST1 | CST6 | NRIP3 | PCSK1 | OR52W1 | TSGA10IP |
| UBQLNL | P2RY2 | PRM1 | LINC00521 | EIF3KP1 | LINC01106 |
| C15orf53 | CREG2 | HOXD8 | DOK7 | LRRN1 | KLHL38 |
| ASCL3 | C11orf16 | TMPRSS7 | RP1-146I3.1 | MC5R | OR11H4 |
| OR51B6 | OR4F21 | IDSP1 | ZNF135 | FOXR1 | RPL37AP8 |
| PFN1P10 | PRR18 | OR5AN1 | PRR15 | MAP3K19 | HORMAD2 |
| NANOGP1 | FOXC2 | ZNF843 | LINC00303 | FKBP9P1 | MIR7-3HG |
| RP11-432M24.4 | GRIN1 | SOX11 | C8orf4 | GCNT4 | TOB2P1 |
| RPL7L1P11 | SCN4B | DSCAML1 | MRVI1-AS1 | LINC00982 | CLVS1 |
| SPACA4 | LINC01561 | ENSAP3 | FLJ40194 | RP4-756H11.4 | ERICH5 |
| GPR4 | IRX3 | RP11-56N19.5 | FAM187B | C12orf54 | C2orf57 |
| SRRM3 | FAM20C | PVRL3 | SLC35G5 | CTD-2530N21.5 | YIPF7 |
| MYOZ1 | CCDC184 | ODF3 | LCN15 | C2orf73 | TSPYL6 |
| ADAMTSL1 | PRSS42 | PPP1R42 | RP4-682C21.5 | RNF212 | AP000345.1 |
| PRM3 | ZNF354B | OR2D3 | DNAJC22 | NEUROG2 | CEP295NL |
| TUBAL3 | MAF | OR6B3 | DEFB125 | OTOS | ACTG1P1 |
| C5orf64 | THBD | LMO7DN | CPN2 | FAM101A | LGALS7B |
| ERICH3 | C14orf39 | KLHDC7A | LL0XNC01-131B10.2 | TRIML2 | OR13D1 |
| IGSF22 | C9orf106 | FAM133A | RP11-458F8.3 | MTUS2-AS1 | GIMAP7 |
| RPS2P28 | GGN | HNRNPCL1 | TMEM151A | WSCD1 | GS1-124K5.9 |
| GATA2 | VWA1 | DNAJB8 | OR6W1P | AC073072.5 | OR9A2 |
| LSMEM2 | RNF151 | GRM8 | RPRML | CDH5 | OR7E22P |
| MRGPRX4 | MYADM | MRGPRX3 | GIPC3 | C1orf194 | B3GNT3 |
| NRXN1 | SSC5D | SPDYE7P | AC079741.2 | FAM71E2 | ANKRD18A |
| C12orf40 | CSNK1A1L | LYNX1 | TH | TDRP | FAM71C |
| TPT1P10 | SLC9A4 | ADGRD2 | PNPLA1 | DEFB124 | KRTAP9-7 |
| CYP8B1 | ZSCAN4 | RNF182 | C9orf139 | FUT7 | MB21D2 |
| RPL21P8 | SHISA2 | CLRN3 | CHST13 | ARSJ | MAP3K15 |
| CXCR2 | GREM2 | OR56B4 | C1orf105 | OR56B1 | FCRL6 |
| SLC26A11 | OR52N4 | MAPK15 | ADIPOQ | SDCCAG3P2 | OR52P1P |
| C4orf50 | HIST3H2A | HSP90AA6P | CCDC108 | AATK | ZNF467 |
| OR6T1 | OR52H1 | TMEM252 | OR4C6 | AP003774.4 | CCDC149 |
| RP11-651P23.2 | PNMAL1 | ADIG | USH1G | TRPC2 | TMEM30B |
| CREB3L2 | UNC5C | LDOC1 | ZAR1 | SYNM | NLRP10 |
| FIGN | IZUMO1 | B4GALNT4 | MBD3L3 | GLTPD2 | KIAA2012 |
| LIPF | PDSS1P1 | RP5-1142A6.8 | IFNL1 | DNM1P46 | OR2B8P |
| TBPL2 | CAV3 | CLEC4G | RP3-341D10.1 | EPHB3 | EPGN |
| OR2V2 | CCNYL2 | NTM | GALR2 | SEPHS1P1 | RGS6 |
| SLC35D3 | HCAR2 | MAGEB17 | CRIP2 | PLCXD3 | VMO1 |
| GALNT9 | ADGRG3 | RP11-558O12.1 | TSPEAR-AS2 | TCEAL7 | ODF3L1 |
| NPM1P14 | ZNF662 | MCEMP1 | PCP4 | FREM3 | BEGAIN |
| FAM43B | GJD3 | CALN1 | GABRR3 | RP5-1120P11.4 | PRR36 |
| NF1P3 | FAM9A | SPDYE4 | REC114 | GBP6 | SYNDIG1L |
| TMEM89 | RIPK4 | ZBTB8OSP1 | SH2D7 | GPR132 | COL18A1-AS1 |
| ACSM5 | PGPEP1L | GOLGA8G | C11orf88 | FAM19A1 | LINC00518 |
| BMP8A | EFHC2 | OR4N4 | LHFP | ZNF703 | SLC35F3 |
| TCEB3C | EMILIN3 | FAM162B | CCR4 | CTA-833B7.2 | ANKRD45 |
| C1orf64 | SH2D1A | ST6GALNAC3 | TMPRSS2 | TREML3P | RP11-366I13.3 |
| RPL7AP28 | KCNJ12 | DGAT2L6 | CDR1 | POU6F1 | PRKD1 |
| CCSER1 | COLEC10 | A3GALT2 | CCR10 | NCMAP | BPIFC |
| POU3F2 | PROS1 | NUTM1 | PTGER4P2 | SOCS3 | RP13-786C16.1 |
| FAM19A3 | ZNF72P | OR7E14P | CLDN6 | FAM110C | MGAT4EP |
| SATL1 | DRD1 | TCEAL2 | CLCNKB | OR6A2 | WT1 |
| SORCS2 | IFNE | SLC22A10 | RFX6 | SLC2A3P2 | SLC24A3 |
| FLRT2 | FAM169B | ZNF479 | LINC00313 | WASIR1 | TNFAIP2 |
| GAPDHP24 | MAGEA11 | ZNF74 | KLHL33 | WBSCR17 | NUPR1L |
| IL3RA | SFTPA2 | C12orf56 | GAS2L1 | OR2V1 | KPNA7 |
| ROR1 | TLCD2 | AC017079.3 | POU3F1 | LYG2 | MORN5 |
| SRL | NLRP9 | NPAP1 | LINC01405 | CCDC190 | TMEM210 |
| TMPRSS11B | THNSL1 | FFAR3 | RTN4RL1 | KRTAP5-5 | CCIN |
| GRK1 | ATP4B | HTR3E | TAL2 | KRT5 | KRT18P14 |
| AGBL4 | ARGFX | PRR30 | UBL4B | CYP4Z1 | BPIFB4 |
| EDARADD | CYP4F12 | MARC1 | SSU72P4 | LCE1E | AC016757.3 |
| GPAT2 | GABRA5 | PRELID2 | SLC36A3 | SLC36A2 | CYP4X1 |
| GLDN | TRDN | TMPRSS12 | FAM228A | AQP7P1 | AKAP14 |
| OR9I2P | CLCNKA | CEACAM19 | SPATC1 | PDE2A | SPDYE17 |
| PRR5 | EEF1GP1 | CYP27C1 | TPI1P3 | FOXE3 | LILRB4 |
| TNFRSF4 | KRT17P2 | CHCHD2P9 | LINC01551 | EMID1 | C17orf82 |
| C3orf70 | TEAD1 | AKR1C1 | SPATA21 | KRT12 | CIDEC |
| DCC | HIST1H1T | CDHR4 | COL4A1 | PLEKHG7 | GJA4 |
| ATP13A5 | AGMO | TLR5 | NANOS3 | TERF1P2 | SAMD11 |
| PERM1 | ERC2 | KBTBD12 | THSD4 | GABRD | AMY1C |
| OR52B6 | C9orf153 | HSPE1P11 | TMEM72 | PEAR1 | RP11-24M17.5 |
| C2orf78 | PALM3 | C1orf168 | AC097382.5 | LRRC74B | DMBT1 |
| OR51I2 | LCN10 | C2orf66 | HS6ST1P1 | PLA2G2C | ZSCAN23 |
| TPRG1 | C1orf204 | RTP5 | C19orf67 | CLCN1 | NRN1L |
| PRSS45 | DAZ1 | TMEM215 | COL4A5 | HEPACAM2 | SMTNL2 |
| RUFY4 | HES4 | SLC38A3 | AC092171.2 | C10orf99 | AC018892.9 |
| PPP3R2 | GPR21 | TCTEX1D4 | NANOS2 | SLC24A5 | AC003003.5 |
| INSC | CFAP77 | FBLL1 | PAQR9 | GOLGA8M | TSPY7P |
| LINC00319 | HILS1 | QRFP | OR13D3P | TBC1D3P2 | FZD9 |
| TMSB4XP2 | OPTC | PLET1 | CATSPER4 | TMCO2 | DPPA3P2 |
| ASTL | GJB3 | USP32P1 | UTS2B | MSANTD1 | LRRC66 |
| SBSN | RELN | APOD | GAGE2A | ANKRD34B | NKAPL |
| OR14I1 | MAOA | AC069277.2 | SPANXN3 | LINC01164 | GJB5 |
| ANKRD62P1-PARP4P3 | RP11-797H7.5 | FAM180A | C6orf222 | FAM179A | KIAA0408 |
| CXCL17 | OR7E12P | USP32P3 | GJB4 | OR2L13 | PTPRT |
| AKR1C3 | OR11H13P | FAT4 | KIF19 | HRCT1 | SIRPB2 |
| KRTAP5-3 | SFTA2 | CGB7 | CPNE4 | ELAVL3 | NUDT11 |
| FUT4 | SLC35F1 | THEM5 | NPIPB15 | RFX8 | ESRRG |
| ARL9 | OR9K1P | CACNA1H | HBG2 | AJAP1 | UGT2B15 |
| HRH1 | ZNF5O2 | SLC30A10 | FAM150A | LCE1B | COL27A1 |
| CTD-2192J16.17 | POTEI | PATE2 | KRT39 | HIST3H2BB | AC090286.4 |
| TEX43 | RP11-88G17.6 | ZNF470 | SIGLEC15 | ZNF300P1 | SLC6A17 |
| SLC22A4 | OR1J2 | FAM110D | TPSB2 | IL27 | RAMP2-AS1 |
| SVIL | OR8S1 | DIO3 | CYP2B6 | HIST1H3D | DCHS2 |
| VEPH1 | PDGFA | AC005276.1 | COL13A1 | ZNF665 | SLC28A3 |
| FAM177B | SSPO | COL4A6 | DMBX1 | ZNF841 | DPP4 |
| PDCD1LG2 | SLC22A24 | PARVA | LHFPL5 | IGKV7-3 | ADAMTSL2 |
| PRB3 | UGT2B17 | HRNR | ZNF311 | ZNF347 | ZNF734P |
| CLEC9A | DLGAP2 | FCGR1B | ZNF560 | TUBA3C | ZNF667 |
| AVPR1B | SIRPA | AKR1B10 | SULT1C4 | CYP2A7 | NUP62CL |
| TMPRSS11F | LPAR1 | SOWAHC | ZNF334 | RP11-566K11.2 | CACNA1E |
| OR5B21 | TMEM239 | ZNF568 | RTP2 | SH3BGRL2 | PLN |
| SLC34A3 | RD3 | RP11-611L7.2 | NUDT16 | MMP17 | ZNF534 |
| TUSC1 | MAGEA1 | LINC01565 | ABCA4 | UNC13B | PPP1R14C |
| SMOC1 | PLXNB3 | EGFL6 | APCDD1L | MUC2 | ALPK2 |
| PAX9 | LRRC10 | SUCNR1 | MTND4LP30 | PNMA5 | SOWAHA |
| MIR210 | RN7SKP184 | SNORA21 | RNU4-39P | RNA5SP477 | RN7SKP173 |
| RN7SKP74 | SNORA17 | RNU1-72P | RNA5SP452 | RNU1-120P | RN7SKP243 |
| RNU4-32P | SNORA71D | SNORA38B | SNORA63 | SNORD118 | RNY1P11 |
| RNU1-124P | RN7SKP180 | SNORA80A | RNU4-1 | SNORA38 | RN7SKP87 |
| RNU4-51P | RNA5SP202 | RNA5S9 | U3 | RN7SKP193 | RN7SKP37 |
| SNORA55 | SNORA71C | RN7SKP237 | SNORA16B | RN7SKP50 | RN7SKP78 |
| RNU12-2P | RN7SKP239 | RNU1-59P | RN7SKP9 | RNU5E-4P | RNU4-8P |
| Y_RNA | RNU1-100P | RNU1-51P | RN7SKP69 | RNU1-16P | SNORA62 |
| RNU1-122P | RNU5E-6P | RN7SKP230 | RNU4-2 | RP11-560A15.3 | Metazoa_SRP |
| RP11-277A4.4 | ZNF525 | RP11-25I9.3 | LINC01562 | LINC01317 | CTD-2026K11.6 |
| RP11-217O12.1 | RP13-36G14.3 | BTBD7P1 | FAM32B | E2F3P2 | Metazoa_SRP |
| HMGB1P39 | TAS2R2P | OR1F2P | RP11-542B15.1 | RP5-1096D14.6 | RHOT1P2 |
| AC012456.3 | LINC01285 | OR2L2 | TCP10 | CAPN8 | LINC00862 |
| SAMD5 | LINC00272 | FCGR3A | FANK1 | KPRP | DDO |
| CCDC162P | PLPP4 | CHIAP2 | CYP17A1-AS1 | RP11-261N11.8 | OOEP |
| C1orf146 | C10orf62 | FAM127B | C1orf141 | GLYATL3 | RP11-188C12.3 |
| LCN8 | TRPC5OS | LRIT2 | LRRC73 | FOXO6 | TDRG1 |
| NEU4 | MAFB | BMP2KL | RP4-640H8.2 | PTPN20 | GDF5OS |
| TXNDC8 | AWAT1 | TCEA3 | OR13C3 | RP11-219G17.4 | FLJ45079 |
| BTNL2 | C6orf10 | CYP21A1P | NXPE2 | RP11-380J14.1 | AC005042.4 |
| LAYN | RP11-410N8.4 | RP11-65D24.2 | CSHL1 | LY6G6C | XXbac- |
| | | | | | BPG32J3.20 |
| LY6G6F | MCCD1 | PSORS1C3 | ZSCAN5CP | AC096670.3 | TRIM10 |
| HCG9 | NMS | RPS26P8 | MOG | OR2H2 | CBY3 |
| C9orf57 | RP5-100419.1 | CTC-281M20.1 | ZNF204P | ST8SIA6-AS1 | PNMAL2 |
| NAT8B | AC021218.2 | GPR20 | SPINK9 | LRRC3C | PSG5 |
| FAM83A-AS1 | ARHGEF34P | BLACE | PCDHA8 | PCDHA2 | PCDHA1 |
| ERICH4 | PRSS1 | AARD | RP11-46C24.3 | SLFN12L | LGALS7 |
| C2orf91 | FAM205A | ACCSL | TRIQK | SLC10A5P1 | SCGB2B2 |
| OR7E36P | KRT17P5 | DGAT2L7P | MUC12 | CTXN3 | RP11-352D3.2 |
| MGAT4D | KRT17P4 | ADGRG1 | MT1H | MT1M | LINC00661 |
| KRT81 | EXOC3L4 | KRTAP10-2 | SPDYE18 | AC013472.3 | RP11-89H19.1 |
| AF064858.6 | LINC00898 | VCX3B | CTD-2375G15.1 | RP3-416J7.5 | ACOT6 |
| CRLF2 | RP9P | LINC01531 | C16orf96 | TTC23L | OR7E91P |
| C22orf42 | FAM99B | KRTAP5-1 | FAM90A2P | RP11-3B12.4 | ONECUT3 |
| RP11-404K5.2 | IFITM5 | C18orf63 | DOK6 | IGLL3P | SERPINB5 |
| CTD-2008L17.2 | HBM | ANKRD20A9P | ANKUB1 | RP11-93O17.2 | VGLL3 |
| PRSS50 | SNORA57 | SNORA22 | SNORA2A | SNORA80B | SNORA22 |
| RNU1-73P | SNORA6 | RNU6-483P | SNORA9 | SNORA29 | RNU4-52P |
| SNORD15A | SNORA76A | Y_RNA | SNORA54 | SNORA27 | SNORA72 |
| SNORA7B | SNORA25 | U3 | SNORA70 | SNORA68 | SNORA37 |
| SNORA70 | SNORA8 | SNORA22 | SNORA20 | SNORA64 | SNORA19 |
| SNORA80E | SNORA46 | MIR7-1 | AL132780.1 | MIR219A2 | MIR671 |
| IGKV6-21 | IGKV3D-11 | IGLV8-61 | IGLV5-52 | IGLV1-50 | IGLV2-33 |
| IGLV3-25 | IGLV3-16 | IGLV2-14 | IGLV2-11 | IGLV3-10 | IGLJ1 |
| TRGV9 | TRGV1 | TRAV13-2 | TRAV9-2 | TRAV22 | TRAV39 |
| IGHV2-5 | IGHV3-7 | IGHV3-13 | IGHV3-15 | IGHV1-18 | IGHV4-28 |
| IGHV3-38 | IGHV3-48 | IGHV1-58 | IGHV2-70 | U3 | U3 |
| SNORA16 | SNORA46 | RNA5SP323 | SNORA74B | SNORA75 | SNORA75 |
| SNORA12 | U3 | SNORA75 | RNA5SP53 | SNORA45B | KRTAP16-1 |
| KRTAP29-1 | KRTAP9-6 | RP11-134K1.2 | AP002414.1 | RPS26P3 | RPL23AP4 |
| POU5F1B | BTF3P12 | PTTG3P | SYT3 | RPL23AP80 | RP11-383G10.3 |
| RP11-532L16.1 | EEF1A1P32 | OR5S1P | HNRNPA1P34 | TPM3P1 | RPL5P5 |
| EEF1B2P7 | RP4-612B18.3 | PFN1P11 | RP11-760D2.10 | yR211F11.2 | FAM106A |
| RP5-933K21.2 | RP11-613F7.1 | ACKR1 | PHBP1 | NPM1P46 | RP3-352A20.1 |
| RP11-306O13.1 | PPP1R14BP5 | ARF1P2 | ELK2AP | RPL23AP60 | RP11-252O18.3 |
| GAPDHP36 | RPL36AP48 | AC016732.2 | ARHGEF35 | RP11-355O1.7 | RPL12P38 |
| ST13P10 | EEF1A1P16 | RBMS2P1 | RPL12P42 | YWHAZP5 | TSGA13 |
| RP11-613M5.1 | RP11-212P7.1 | RPL18P11 | RPL9P18 | CYCSP19 | RPL7AP34 |
| ATF4P1 | AC092933.4 | RP4-778K6.1 | NAP1L1P3 | LINC00671 | GAPDHP71 |
| RP11-779O18.2 | RP11-66N11.6 | RPSAP46 | ANXA2P1 | HNRNPA1P33 | CTB-47B8.5 |
| KRTAP4-12 | KRT222 | YBX2P1 | H2AFZP1 | VDAC1P7 | HMGB1P37 |
| FIRRE | RP11-972K6.1 | EIF4A1P8 | ACTN4P1 | TPT1P13 | GBP7 |
| AC132008.1 | SETP8 | AC005522.6 | AC005077.8 | RP4-595K12.1 | KRT18P19 |
| AKR1B10P1 | RP11-380G5.3 | BOLA3P3 | CTD-2158P22.1 | OLA1P2 | SLC25A5P3 |
| SEC14L1P1 | LA16c-60G3.7 | RPS10P14 | KB-1683C8.1 | HNRNPA1P32 | PSMC1P2 |
| RP11-941H19.2 | AC004129.9 | RPL13P2 | TAF9BP1 | RPL7AP64 | RPL3P6 |
| RP11-546M4.1 | NPM1P33 | HBG1 | AC092610.12 | RP11-94D20.1 | RP1-102E24.1 |
| KRT18P17 | ITGA1 | KRT18P29 | API5P2 | RP11-264F23.1 | RPL7AP14 |
| AC007277.3 | RP11-414H17.5 | ATP5F1P3 | KRT18P38 | RP11-414H17.2 | ARPC3P5 |
| RP11-169L17.3 | AC073310.4 | PGAM1P4 | IFNA7 | RPL6P5 | RP11-549L6.2 |
| RPL23AP39 | CYP4F30P | SMCO1 | WEE2 | RP1-258N20.3 | TMEM213 |
| ACTN4P2 | RP11-488L18.1 | RP11-699L21.1 | RNF152P1 | C12orf74 | SETP21 |
| PUDPP3 | RAD51AP1P1 | AZGP1P1 | OR1X5P | AC133644.3 | NEURL1B |
| AC009967.3 | RP11-457K10.2 | LCNL1 | GOLGA2P8 | SEC14L6 | HIGD1C |
| KRT42P | AC130709.1 | AP000343.1 | AC087491.2 | MEG3 | POLR3DP1 |
| RP11-392M9.2 | IQCF6 | AC090616.2 | CDIPT-AS1 | RPL5P35 | RP11-429J17.8 |
| RP11-54I5.1 | AC004870.5 | CTD-2509G16.1 | HNRNPA1P15 | RP5-1053E7.3 | HMGB1P4 |
| RAD51AP2 | RP11-115L11.1 | APOC1P1 | LINC00243 | RP11-229M1.2 | RPL23AP3 |
| RP11-674E16.4 | SPATA31D1 | TBC1D26 | ISPD | RP11-274J2.1 | RPL7AP26 |
| HSPD1P7 | AC114730.5 | PRDX2P1 | PKMP5 | DUSP8P4 | DUSP8P3 |
| RP11-30M20.1 | NLRP2P | MUC5AC | GOLGA6B | UBE2QL1 | LA16c-60G3.8 |
| RP11-62E14.2 | NPM1P12 | ZNF705D | RP11-754I20.1 | CTAGE12P | RP5-890O15.3 |
| BCRP4 | RPL27AP | FAM230B | LINC00189 | TTTY5 | CNN2P7 |
| GAB4 | RP5-967N21.2 | C20orf202 | KRT18P35 | GCGR | CELA2B |
| LRRC37A14P | RP11-364B6.2 | FEN1P1 | MARCKSL1P2 | RPL23AP17 | C1orf167 |
| MMP23A | KRT19P2 | RP11-360O19.1 | MRPL42P2 | RPL12P2 | RAET1M |
| LINC00955 | GAPDHP72 | RPL7P25 | RP3-355L5.3 | RP11-812I20.2 | TUBB2BP1 |
| AL022393.7 | AC131097.4 | RP3-329A5.1 | RP1-13D10.3 | RP1-245M18.2 | LARP1P1 |
| RP11-360O19.5 | RP1-319M7.2 | RP1-182O16.1 | AC018865.5 | OR2W2P | SSXP10 |
| RP11-474L11.5 | RP11-477E3.2 | RPS16P5 | IQCB2P | PTGES3P2 | RP4-803A2.1 |
| HIST1H2BPS2 | RP1-95L4.4 | CCT7P1 | RP1-303A1.1 | RP11-160A9.2 | AC099552.4 |
| HIST1H4PS1 | OR4F7P | KRT18P48 | HSPE1P8 | CICP18 | ZNF192P2 |
| PRKRIRP5 | KRT18P22 | KRT8P43 | POM121L14P | RP11-367G18.2 | RP11-393I2.2 |
| HNRNPA1P1 | NIP7P3 | H2AFZP3 | RP11-12A2.1 | GAPDHP63 | RP11-2J18.1 |
| RPL7P28 | RPL5P20 | AC010150.1 | RP1-34L19.1 | RP3-453I5.2 | RP3-340B19.5 |
| HSPD1P16 | TDRD15 | FAM138C | RP1-209B5.2 | CNN3P1 | RP11-528A10.1 |
| SOD1P1 | RP11-214N16.2 | AC011298.2 | HMGB1P20 | RPS20P2 | RP11-397G5.2 |
| RP11-491H9.3 | RP11-106E7.1 | TEX40 | RP11-3B12.3 | RPL17P25 | ZNF259P1 |
| HIGD1AP16 | RP11-262H14.11 | RP11-532F6.2 | RP1-199J3.5 | VN1R11P | RPSAP45 |
| ATP5F1P6 | RPL35P3 | RP11-394A14.2 | LAP3P1 | RP11-307I14.2 | AF238380.8 |
| HNRNPA1P12 | FLJ35934 | RPS24P12 | RP11-230C9.1 | RP3-431A14.4 | KRT8P42 |
| RP3-520B18.1 | YAP1P1 | RP1-177A13.1 | TUBBP9 | RP3-509I19.6 | RP3-406A7.3 |
| RP11-277I20.3 | RP1-92C4.1 | IGBP1-AS2 | HNRNPA1P41 | U3 | SNORA11 |
| SNORA81 | SNORA34 | SNORA3 | AL590226.1 | MIR1307 | C7orf65 |
| CTD-2527I21.4 | PLXNA4 | HMSD | NPTXR | HEPN1 | TAS2R40 |
| PRR21 | TMEM14EP | TTC4P1 | LINC01118 | PSMB11 | LINC01124 |
| AC079354.3 | POTEM | POTEJ | RN7SKP285 | RNU2-65P | RN7SKP16 |
| SNORA73 | RN7SKP151 | RNU2-17P | RN7SKP114 | RNU2-33P | RN7SKP221 |
| RN7SKP158 | RNU2-28P | RN7SKP271 | SNORA79 | RNU4-25P | RN7SKP235 |
| Y_RNA | RNU2-70P | RN7SKP123 | RN7SKP38 | RNU2-7P | RNU2-68P |
| RN7SKP259 | SNORA57 | RN7SKP165 | RNY3P8 | RN7SKP30 | RN7SKP273 |
| KLF2P3 | RP11-224O19.5 | RP11-374M1.4 | RP11-408H1.3 | RP5-858B6.3 | AC016716.1 |
| RP4-800M22.2 | RP11-274J16.5 | RP11-52I18.1 | RP11-285G1.9 | AC064834.2 | RP11-310H4.1 |
| RP4-788P17.1 | MAPK6PS2 | CHCHD2P5 | RP11-15M15.1 | RP11-763B22.7 | RP11-397P13.7 |
| RPL23AP53 | RP13-297E16.4 | KIF28P | EEF1A1P8 | RP11-24F11.2 | SMPD4P1 |
| TNRC18P2 | RP11-275I14.2 | CKMT1A | RBMX2P3 | RP11-61K9.2 | RP3-476K8.4 |
| AL356806.3 | RP1-292B18.4 | EBLN1 | AC125634.1 | MTCO1P17 | AK4P4 |
| AC008277.1 | RP11-131L23.1 | AC012314.20 | CTB-111F10.1 | EEF1A1P24 | RP11-93B14.4 |
| IFNWP18 | ADH5P3 | RAET1E-AS1 | SFR1P2 | RP5-1172N10.2 | LINC01208 |
| RAD17P2 | AC007879.5 | RP11-145E17.2 | RP11-321C24.1 | RP11-644K8.1 | AC007389.2 |
| PSMD4P1 | MIR503HG | AC008073.9 | MED15P9 | RP11-54O7.3 | RP11-307B6.3 |
| LGALS8-AS1 | RP11-403I13.4 | RP4-593M8.1 | BOLA3P4 | KRT8P28 | AC004870.4 |
| AC008074.4 | HLA-DPB1 | AC006372.5 | AC005517.3 | GNL2P1 | RPL23P4 |
| AC068657.2 | ZNF32-AS3 | EEF1A1P36 | AC079630.2 | DPPA2P1 | RP11-353H3.1 |
| RP11-430G17.1 | RP11-458I7.1 | PRKRIRP8 | ZNF587P1 | AC002456.2 | DDX11L1 |
| BNIP3P42 | SMCR2 | RP11-528A4.3 | AC104809.2 | LL22NC03-102D1.18 | RP3-471M13.2 |
| AC019070.1 | RP11-488L18.6 | RP11-728K20.1 | RPL21P32 | RP11-274B18.3 | AC091493.1 |
| IGKV3D-15 | RP11-30O15.1 | EGFR-AS1 | RP11-430K21.2 | AC009487.4 | AC091729.7 |
| LINC01057 | RPL23AP41 | AP001630.5 | ATG12P2 | POM121L10P | DPPA2P2 |
| MIR548XHG | AMMECR1-IT1 | RP11-309L24.6 | HAGLR | RP11-10L12.1 | AL590762.6 |
| LINC00466 | AC104699.1 | OR2L1P | AC110299.5 | MRRFP1 | WARS2-IT1 |
| AC090044.2 | LINC00403 | LINC01133 | RPSAP18 | RP11-191L9.4 | AC005077.7 |
| ENSAP1 | MSL3P1 | RPL7P7 | RPL12P14 | DPRXP1 | AP000357.4 |
| RP11-496H15.2 | BICD1P1 | RP11-462G2.2 | MTND2P5 | RP11-252P18.1 | RP11-814E24.3 |
| RP3-417O22.3 | RP3-395M20.2 | LINC01280 | LINC01272 | OR6D1P | RP5-956O18.3 |
| AC010886.2 | SLC31A1P1 | MKRN5P | RPL23AP14 | AC017035.2 | ATP5F1P1 |
| RP11-439L18.2 | PGAM1P6 | CDCA4P2 | AC009313.1 | AC073900.4 | RP11-495P10.3 |
| TTC21B-AS1 | RPL36P4 | MAPKAPK5P1 | KRT8P45 | PPIAP10 | AC008264.4 |
| RP11-370B11.3 | HMGB3P21 | AC008065.1 | RP11-438N5.2 | RP11-430H12.2 | AC012314.19 |
| RP11-365D23.2 | RP5-884C9.2 | RP11-30B1.1 | ZMIZ1-AS1 | RPS5P2 | BMS1P12 |
| AC096921.2 | AC106053.1 | PDSS1P2 | AC106900.6 | RP11-397D12.4 | AF124730.4 |
| IGHV3-74 | LINC00885 | PDIA3P2 | GAPDHP46 | ZNF812 | UBE2D3P3 |
| RPS17P13 | OR11M1P | AC025165.8 | CITF22-49D8.1 | RP11-576D8.4 | TEX26-AS1 |
| RP11-380G5.2 | VN1R21P | LINC01167 | CR769776.3 | MUC20P1 | RPSAP50 |
| AC006026.9 | AP000704.5 | KRT18P39 | RP11-445O16.3 | RP11-235D19.2 | TUBB4BP2 |
| LINC00853 | BEND3P2 | SEPT14P13 | RP11-307E17.9 | RP11-134G8.10 | LINC00265-2P |
| OPA1-AS1 | GXYLT1P2 | MTCYBP29 | AC007899.3 | VPS26BP1 | RP11-3B12.5 |
| TIMM8BP2 | KRT8P30 | LINC00184 | RP11-82L18.2 | PSAT1P4 | RP11-403H13.1 |
| LARGE-AS1 | RP11-160H22.3 | RP13-228J13.9 | RP11-409K20.7 | RP13-228J13.6 | RP13-348B13.2 |
| AP004289.1 | AC091492.2 | AC096574.4 | RP11-132N15.1 | RP11-383G10.5 | GS1-184P14.2 |
| RP11-426L16.3 | WWC3-AS1 | RP11-123N4.4 | DAP3P1 | AL450226.2 | RP11-15B24.1 |
| SNORA71A | RP11-405O10.2 | ATP6V1E1P1 | AC083900.1 | RPL23AP36 | RANP4 |
| MORF4L1P4 | PGBD4P5 | AC073957.15 | AC012354.6 | CTD-2538A21.1 | BRI3P1 |
| RP5-828K20.1 | OSTM1-AS1 | RP11-795A2.2 | ATP5LP4 | AATK-AS1 | RP11-388K2.1 |
| RP11-481K9.4 | ZNF33BP1 | LINC00092 | RP11-197M22.2 | SMARCE1P1 | AC007362.3 |
| HSPA7 | RPL21P10O7 | RPS2P1 | ZNRF2P2 | RP11-466F5.4 | RP11-536C5.2 |
| AC018693.6 | AC005105.2 | MTND4P4 | RP11-344A5.1 | RP11-1086F11.1 | RP11-512N4.2 |
| LINC00350 | LHFPL3-AS2 | HMGB3P1 | RPL21P66 | SNX18P3 | RP11-820K3.2 |
| RPF2P1 | MIPEPP1 | PPP1R26-AS1 | RP11-3B7.1 | AC010878.3 | RP11-207C16.4 |
| RP3-509I19.1 | AC019330.1 | AP001626.1 | LINC01504 | RP4-665J23.2 | RP13-93L13.1 |
| ZNF70P1 | RFPL1S | RP11-262D11.2 | RP11-120J1.1 | RP11-38J22.2 | GBP1P1 |
| AC104651.2 | RPL15P4 | RP11-330C7.3 | CYP4A22-AS1 | AC005237.4 | PAWRP1 |
| LINC01393 | ZNF385D-AS1 | RP11-210H10_A.1 | AP000320.6 | UBE2D3P2 | FAM197Y8 |
| RP11-384B12.3 | DRAXINP1 | EDNRB-AS1 | LINC01623 | MTOR-AS1 | RP11-325P15.1 |
| RP11-70C1.1 | ZNF469 | RP4-604A21.1 | HNRNPA3P15 | PABPC1P12 | RP11-70P17.1 |
| AC005487.2 | EIF2S2P5 | RPL14P5 | FCF1P6 | CCNT2P1 | AL163953.2 |
| FAM225B | AC005522.7 | PTPRD-AS1 | RP4-742C19.12 | FAM66E | AC007000.10 |
| AP001627.1 | NPM1P18 | LL22NC03-88E1.17 | AC017002.4 | AL773572.7 | RP11-267N12.2 |
| RP11-417O11.5 | LINC01389 | DHRS4L1 | RP4-603114.3 | OR6V1 | AC073321.4 |
| MTND4P23 | WARSP1 | UBXN7-AS1 | RPS18P1 | NIPA2P1 | RP11-46A10.2 |
| LINC01529 | LINC00694 | BMS1P11 | RP11-288L9.4 | RP11-6J24.3 | HSPE1P13 |
| RPS27P16 | AC009310.1 | CACYBPP1 | RP3-395M20.7 | CTAGE4 | RP4-566D2.1 |
| PCA3 | RP4-575N6.4 | C5orf67 | NTF4 | NRIR | RP11-192N10.2 |
| AC010746.3 | AC102953.4 | HMGB1P26 | RPL23AP34 | RP11-312J18.3 | SEPT14P3 |
| RP11-403E24.1 | COX5BP7 | RP11-57H12.3 | CDY10P | YBX1P8 | ZNF503-AS1 |
| MTND4P32 | RP11-24H1.1 | SLC39A12-AS1 | EIF3LP3 | LINC00937 | SEPT7P3 |
| AC111200.8 | AC098823.3 | RP1-228P16.7 | RP1-138B7.5 | RPS27AP3 | SLC25A39P1 |
| LINC01375 | LINC00685 | RP11-328D5.1 | AC007790.4 | RPS12P5 | RPLP0P1 |
| LEMD1-AS1 | RP11-276H19.1 | RP1-310O13.7 | LL0XNC01-221F2.2 | RP1-18D14.7 | PSPHP1 |
| RPL12P10 | RP1-80N2.2 | OR52I2 | RP11-528N21.1 | SMARCE1P2 | RP3-323P24.3 |
| LINC00351 | CROCC2 | RP11-217B7.2 | AC083822.2 | HAGLROS | AC102948.2 |
| ST7-AS2 | RP11-358D14.2 | MRPL23-AS1 | IGLV3-4 | AC093642.4 | HMGN2P7 |
| RP11-111F5.6 | RP11-1L9.1 | AC018641.7 | RP11-551L14.4 | OR7H1P | RPSAP21 |
| USF1P1 | AC104655.3 | UPK1A-AS1 | FAM138B | AC126365.1 | AC074375.1 |
| BTBD9-AS1 | RP11-417L14.1 | MYL6P1 | RP5-1092L12.2 | NPM1P49 | TRAF6P1 |
| CYP4F26P | RP11-505O17.1 | RP11-258F22.1 | UBE2V1P5 | IFNWP9 | AC017060.1 |
| PAPPA-AS2 | AC083855.5 | AC092155.4 | LINC00974 | UBE2V1P1 | RP13-13A3.1 |
| RP11-128M1.1 | AP006216.10 | AC019118.4 | OR13Z1P | RPL23AP30 | CHMP1AP1 |
| RP11-526D8.7 | RP11-509J21.4 | LINC01108 | PWWP2AP1 | NPM1P4 | SMCR5 |
| DISC1-IT1 | TAS2R46 | FABP7P1 | GAPDHP54 | RP11-654E17.2 | TBCAP1 |
| TLK1P1 | RP1-167F1.2 | OSTCP8 | AC011893.3 | RP5-881L22.5 | RP3-419C19.2 |
| RP11-278H7.1 | PAICSP3 | AC004112.4 | DUTP3 | RP11-569A11.1 | AC002472.11 |
| RP11-575L7.2 | RP4-782L23.2 | LA16C-395F10.1 | LINC01359 | RP11-298J20.3 | HIST1H2BPS3 |
| ISCA2P1 | NRBF2P2 | LINC00161 | RBMY1J | ALG1L5P | RP4-714D9.4 |
| RP4-533D7.4 | MTCO1P21 | AC003088.1 | ABHD17AP6 | RP4-591B8.2 | RP11-544A12.5 |
| AL049758.2 | FUNDC2P4 | RP11-234N17.1 | LINC00511 | GTF2IP7 | RP11-395B7.4 |
| RPL6P2 | ANHX | RP3-340N1.2 | RP11-12A16.3 | FOCAD-AS1 | RP3-466I7.1 |
| AC107983.4 | RP11-460N20.4 | RP3-471C18.1 | RPL12P44 | RP11-545A16.3 | DDX11L16 |
| PGCP1 | RP11-6C10.1 | RP1-45I4.3 | MIR663AHG | AC009518.8 | RP11-121A14.3 |
| CNN2P1 | RP5-1100E15.4 | SPATA13-AS1 | CICP17 | TTC39DP | RP11-330M2.3 |
| YWHAZP7 | RP11-96L7.2 | KRT16P2 | RP11-95I16.2 | RP1-228H13.1 | RP11-258C19.4 |
| THRAP3P1 | MTND5P25 | AC079753.5 | PPIAP2 | RP11-571F15.2 | HPN-AS1 |
| RNF6P1 | AC008746.3 | AMYP1 | ZMYM4-AS1 | RP5-1042K10.12 | RP11-109P14.2 |
| RP11-397P13.6 | MRPS21P2 | AC069213.4 | FCF1P1 | MTND4P30 | RP11-158D2.2 |
| LINC01537 | AKR1B15 | RP11-295P9.2 | CTA-342B11.1 | RPS20P15 | RPL7P6 |
| RP3-323B6.1 | SFR1P1 | AC018647.3 | RP11-139K1.2 | LINC00376 | RP13-140E4.1 |
| RP11-119F7.3 | RP11-162A23.5 | ATP5G1P5 | RP1-28O10.1 | RP1-167A14.2 | CERS6-AS1 |
| RP11-492E3.2 | PHBP11 | RP11-431K24.3 | SOX21-AS1 | RP11-522L3.9 | ISCA1P6 |
| UST-AS1 | CYCSP24 | LINC01068 | AC087499.10 | HNRNPA3P2 | RP1-266L20.4 |
| MTND6P18 | AC006042.6 | RP4-565E6.1 | CALR4P | AP000344.4 | VTI1BP4 |
| AC072062.3 | RP4-738P11.3 | AC007365.4 | RP11-248J23.5 | FAM212B-AS1 | RP1-180M12.1 |
| MRPS17P9 | AC008850.3 | AC093166.2 | RP11-184B22.2 | SEPT2P1 | DPY19L2P3 |
| C1orf234 | PSPC1P1 | FAM66A | OR5P4P | RP11-141M1.1 | RP11-143M1.3 |
| RP6-102O10.1 | UQCRBP2 | AC003989.4 | RP11-312B8.2 | MTND5P1 | DHX9P1 |
| RP11-350G8.5 | ZNF680P1 | RAPGEF4-AS1 | HNRNPA1P46 | HCG20 | AC069257.8 |
| AC019084.7 | HSPD1P9 | RP4-781K5.4 | RP11-15B24.2 | RP11-151F5.2 | RP11-69E11.8 |
| Z83001.1 | BOD1L2 | IFNA14 | PNKDP1 | AC003991.3 | RP11-15B24.3 |
| AC099684.1 | LINC01494 | RPL3P1 | RP11-84A14.4 | AC004941.3 | RP13-57D9.3 |
| LL22NC03-23C6.15 | AC097713.3 | RP11-455B2.9 | RP11-363H12.1 | PTPN2P1 | AL022341.3 |
| RNF144A-AS1 | LINC00693 | AP001476.4 | EFCAB14-AS1 | GS1-304P7.2 | MOB1AP1 |
| RP11-127L20.3 | ARMCX3-AS1 | AP000593.5 | RP11-220I1.4 | RP4-686C3.7 | BMS1P17 |
| GAPDHP45 | RP11-522L3.5 | RP11-730A19.5 | RPL17P43 | MIR646HG | RPS26P4 |
| CELSR3-AS1 | RP11-365F18.1 | AC106873.4 | TXNDC12-AS1 | RP5-1125A11.4 | RP4-575N6.2 |
| AP001468.58 | RP1-111D6.3 | CDY4P | RP11-549L6.3 | RP4-633I8.4 | RPS15AP9 |
| RP11-176D17.3 | U51244.2 | GRK5-IT1 | LINC01013 | AC006460.2 | RP11-339B21.8 |
| RP11-281A20.2 | RP11-236F9.2 | RP5-1087E8.2 | AC073326.3 | RP3-340B19.3 | RP11-114M1.1 |
| AC013436.6 | MACC1-AS1 | RPL7P52 | RP5-872K7.7 | AC093166.3 | ZNF883 |
| RP11-495P10.9 | HOTAIR | RP4-534N18.2 | AC005152.3 | AC008940.1 | RPL23AP58 |
| RP11-791O21.5 | RP11-20O24.1 | KRT18P1 | TRGV5P | TTC3-AS1 | RP4-676J13.2 |
| RP11-492I21.1 | RP5-826L7.1 | RP11-155G14.1 | AP001065.15 | RP11-316K19.3 | AC008781.7 |
| RP13-39P12.3 | LIMS1-AS1 | AC007327.6 | RP11-336N8.3 | LINC00266-4P | HCG22 |
| AC113607.2 | RP11-253D19.1 | AC073641.2 | LAMA5-AS1 | MARK2P13 | RP1-305B16.3 |
| RP11-293F5.8 | CT45A5 | RP11-112J3.15 | ATP5G2P4 | AC096664.2 | RP11-439E19.6 |
| AC008281.1 | OR1H1P | AP000355.2 | AC002069.5 | SNRPD2P1 | AC004485.3 |
| SLC25A6P5 | SATB1-AS1 | HCG23 | UBTFL6 | RP11-107G24.3 | EXOSC3P1 |
| RP5-979D14.1 | RP11-224O19.4 | RP11-88I18.3 | AC067945.3 | HMGN1P2 | DUTP1 |
| AC074338.4 | TRBV23OR9-2 | PSMA6P2 | LINC01548 | OR51AB1P | AC068491.2 |
| AGBL5-IT1 | ACTG1P2 | SNX18P4 | TCP1P1 | AC108059.4 | RP11-775D22.2 |
| AC103564.7 | LINC00200 | KCTD9P6 | LINC00582 | AC022201.4 | PPP1R12BP1 |
| PNPT1P1 | AC098973.1 | LINC00377 | RP11-552D8.1 | LRRC37A12P | RP11-227H15.4 |
| AC004691.5 | AC091493.2 | RP11-483F11.7 | RP1-40E16.2 | RP4-580N22.2 | AC069154.4 |
| RP11-135A24.2 | AC006947.1 | GK-IT1 | RP11-193I22.2 | AC007563.3 | RP4-639J15.2 |
| SZT2-AS1 | LINC00452 | AC147651.5 | MIR5689HG | RP11-494O16.3 | SFTA3 |
| RPL27AP8 | AC007349.4 | ANKRD26P4 | THEMIS3P | LYST-AS1 | ACTG1P11 |
| RPL39P18 | AC105053.3 | AC092155.1 | MYCBP2-AS2 | AC053503.4 | AC079776.1 |
| RP1-153P14.3 | RP4-717I23.2 | RPL21P44 | MYL8P | AL590762.10 | RP11-390F4.10 |
| MTND5P2 | METTL21AP1 | AC007879.7 | RPSAP22 | LINC01150 | AC007327.5 |
| AC114755.5 | RP11-305L7.6 | AC011242.5 | KARSP1 | EEF1DP3 | RPL23AP19 |
| RP11-556N4.1 | U91324.1 | RP11-567B20.2 | RP11-142M10.2 | DYNLT3P2 | RP4-644L1.2 |
| AC013444.1 | AC118754.4 | SNRPFP2 | RPll-522L3.11 | XIST | CCNB1IP1P1 |
| RP1-308E4.1 | RP5-1174J21.2 | AC093899.3 | RP5-837O21.4 | PGA3 | RP11-505P4.7 |
| RP11-363N22.2 | OGFR-AS1 | EIF1P1 | PAICSP5 | HNRNPA1P6 | RP11-206L10.4 |
| AC016999.2 | RPS4XP5 | RP1-151F17.1 | PRPS1L1 | RP11-111F16.2 | RP11-93N20.1 |
| B3GALNT1P1 | FLJ31356 | RP11-135A1.3 | RP5-1119A7.10 | LINC01435 | RP11-15I11.2 |
| RP11-574K11.8 | AKR1B1P1 | HMGB3P9 | SNAP47-AS1 | RP4-568F9.6 | RP11-217B7.3 |
| RP11-481H12.1 | FNTAP2 | RP11-95P13.1 | CDY11P | BIRC6-AS1 | AC017104.4 |
| RP11-402G3.5 | TRPM2-AS | RP11-384C4.6 | CDC42-IT1 | MTAPP2 | STK24P1 |
| TMEM254-AS1 | RP11-407B7.1 | AC018712.2 | RP11-431N15.2 | CTA-126B4.7 | RP11-275N1.1 |
| AC091177.1 | RP11-163G10.2 | EEF1A1P39 | RP11-282I1.1 | RP4-539M6.18 | RP11-305L7.5 |
| AC018463.4 | ATP5G1P1 | HNRNPA1P28 | LINC01149 | OR4F3 | AC005518.2 |
| ATP6V0CP1 | HSPB1P2 | RP11-173G21.1 | FMO7P | RP11-108M9.2 | RAD23BP2 |
| NFE4 | AC002539.1 | MIRLET7DHG | HERC2P4 | RP1-40E16.9 | PGAM1P3 |
| RP11-213G2.2 | CICP6 | ELOVL2-AS1 | FEZF1-AS1 | BEND7P1 | RP3-323N1.2 |
| RP13-455A7.1 | NCAPD2P1 | RPL23AP52 | KARSP2 | RP5-1139I1.1 | AC018717.2 |
| AC007163.6 | RP5-902P8.10 | LINC00856 | XX-C2158C6.1 | RP11-313A24.1 | AC114803.3 |
| CTF2P | NEK2P4 | ANKRD18B | U73166.2 | RP11-741G21.1 | AP000695.6 |
| ATP5G2P3 | AC124057.5 | OR51A10P | AC108463.1 | COL6A4P1 | RP11-297A16.2 |
| BASP1P1 | RP11-379C10.4 | RP11-305L7.1 | LINC00102 | HMGB1P11 | USP17L1 |
| AC092162.1 | AC114763.1 | AC027612.3 | OR6R1P | RP13-228J13.5 | CCT5P2 |
| AC093609.1 | RPSAP8 | RP11-402G3.3 | TSEN15P2 | RP11-469A15.2 | RP4-781K5.6 |
| DNM3OS | RP11-359N11.1 | RP5-996D20.3 | RGPD4-AS1 | PRB4 | PRKRIRP1 |
| CEACAM22P | AC067945.2 | SAP18P3 | RP11-681L4.1 | LINC00332 | GGTLC4P |
| RP11-14C22.6 | RP4-612B15.2 | RP11-296L22.8 | RP11-478K15.6 | AC007036.4 | ZNF341-AS1 |
| SNAP23P | AC009961.2 | PSAT1P3 | ARHGAP26-IT1 | SMARCE1P5 | RP11-243J16.8 |
| C2orf27AP3 | SRGAP2-AS1 | KB-288A10.17 | RP11-417B4.2 | CTD-2186M15.1 | AC007349.7 |
| RPEP3 | AP001187.11 | RPL31P2 | GOT2P2 | LINC00486 | FAM204CP |
| AC004840.8 | TMBIM7P | RP11-395C3.1 | MIR205HG | RP11-314C16.1 | RP3-452M16.1 |
| RPL12P15 | MTND5P8 | RP11-346K17.3 | OR52B5P | RP13-26D14.2 | AC105402.4 |
| RP4-760C5.3 | AC008154.4 | RP11-298J23.5 | PRELID3BP11 | RP1-34H18.1 | HLA-T |
| ARHGAP42P2 | RPL7P56 | EEF1B2P1 | RP11-38L15.3 | RP11-34D15.2 | RP11-462B18.2 |
| FARP1-AS1 | ZBTB46-AS1 | LINC01510 | GS1-600G8.3 | RP11-1M18.1 | TRIM31-AS1 |
| LINC00349 | RPS3AP3 | PSMC1P3 | PCED1CP | C17orf105 | IGKV1OR2-108 |
| EZH2P1 | TBL1XR1-AS1 | RP11-310H4.6 | RPL34P6 | AC017006.3 | VDAC1P6 |
| RP11-444C12.1 | AC006028.11 | AC072052.7 | HMGN2P16 | NDUFAF4P4 | RNF2P1 |
| AC007919.19 | RP11-310H4.3 | RAC1P3 | RP11-83J16.1 | AC016700.6 | AC006050.2 |
| LINC01516 | RP11-343N15.2 | FAR2P4 | RP1-14D6.7 | AC097359.2 | RNASEH1P2 |
| RP5-1170K4.7 | AC141930.2 | RP4-535B20.1 | LINC01347 | AC007285.7 | RP11-187A9.3 |
| HINT1P1 | FBXO36-IT1 | RPS6P2 | RSL24D1P11 | OR55B1P | PTCHD3P2 |
| RSL24D1P3 | AC091814.3 | RP11-245P10.4 | Z95114.3 | LINC01363 | AC021021.1 |
| RP5-943J3.1 | RP11-296O14.2 | CTD-2104P17.1 | LINC00283 | ATP5BP1 | AGBL5-AS1 |
| GHc-210E9.2 | FSIP2-AS1 | AC011742.3 | RPS8P6 | OGFOD1P1 | OR7E111P |
| RP1-27K12.2 | NANOGP5 | RP11-367J7.4 | AC004895.4 | AF064860.7 | COX6CP2 |
| VN1R110P | ARHGEF9-IT1 | RP11-164H5.1 | RP11-183I6.2 | AC092652.1 | LINC01354 |
| AC090960.1 | RPL31P50 | RP11-317B17.3 | LINC01388 | LINC01198 | C18orf42 |
| AC016735.2 | RP11-216N14.5 | XX-FW83128A1.2 | HMGB3P14 | AC067945.4 | AC007875.2 |
| OR5K2 | AP001625.5 | NDUFB1P1 | MTND5P15 | RP11-29B9.2 | SETP10 |
| GAPDHP37 | RARRES2P3 | AC007682.1 | HS1BP3-IT1 | RP4-706G24.1 | GNAI2P2 |
| RP5-967N21.7 | RP11-12D24.6 | RP11-452K12.7 | LINC00202-2 | AC011899.10 | RPL7L1P12 |
| RP11-57H12.2 | CAP1P2 | RP11-571E6.4 | AC074183.4 | FAAHP1 | AC007391.2 |
| ZBED9 | RP11-45J1.1 | LINC01105 | RP1-302D9.3 | MRPL35P2 | LINC01031 |
| LARGE-IT1 | RPS6KA2-IT1 | RPL31P7 | AC079781.8 | AL022344.2 | RP11-149I23.3 |
| RP13-30A9.1 | LINC00867 | RPLP1P13 | RP11-100G15.2 | RP1-313L4.3 | TOMM22P2 |
| AC098824.6 | RP3-477M7.5 | OR2L6P | FTLP8 | RP1-149A16.16 | LINC00202-1 |
| CDY3P | ASCL5 | BTF3L4P1 | XXyac-YX155B6.5 | OR52I1 | IFNWP15 |
| MTND1P32 | RP1-292B18.3 | AC012485.2 | RP11-557H15.4 | RP1-249I4.2 | AC009299.5 |
| RP11-395C17.1 | RPS27AP2 | RP11-46O21.2 | SEMA3B-AS1 | ATP5LP2 | RP1-102D24.5 |
| RP5-882O7.4 | MTCYBP3 | RP11-308D16.1 | OR52U1P | NDUFA5P1 | RPL5P6 |
| TMPRSS11CP | RAD17P1 | RP11-511111.1 | RP11-234K24.3 | AC009495.3 | CTB-51J22.1 |
| RPL31P15 | GXYLT1P3 | RP11-103C3.1 | RP11-487I5.4 | RP11-416N4.1 | RP4-794H19.1 |
| LINC01450 | RP11-337A23.5 | RP11-241F15.7 | TGFB2-AS1 | RASA3-IT1 | SAPCD2P3 |
| RPL12P11 | MRPL45P1 | VDAC1P13 | RP4-673D20.3 | RP1-257I9.2 | RP11-63K6.7 |
| SRD5A1P1 | RSU1P2 | RP4-591N18.2 | RPL23AP35 | CTC-303L1.1 | LL22NC03-22A12.12 |
| LINC01412 | TIMM9P2 | RPL23AP31 | RP11-175B9.2 | RP11-365O16.3 | HLA-DQB2 |
| MTND2P23 | RP11-342C20.2 | AC008073.7 | FAM136BP | ACTG1P21 | ATP11A-AS1 |
| LINC00442 | HSPE1P9 | AC012370.2 | AC017006.2 | NUTM2HP | RP4-669P10.16 |
| AC016831.6 | TRIM80P | YWHAEP1 | AC007551.2 | RP11-767C1.1 | RP11-15B24.4 |
| RP11-177A2.5 | RP1-85F18.6 | ETF1P1 | RP4-784A16.4 | RP11-498B4.5 | RPL23AP50 |
| OR11I1P | FTH1P25 | HNRNPDP1 | RP11-204M4.1 | PRR13P1 | GSTO3P |
| LMLN-AS1 | AF131217.1 | RPL34P27 | RP4-665N4.4 | TRBV29-1 | RP11-544M22.3 |
| MTND6P24 | USP12PY | RP11-98D18.2 | RPL23AP87 | RP11-390F4.2 | IPO7P1 |
| HSPA8P18 | LINC00374 | HMGN2P20 | POLHP1 | CYP1B1-AS1 | RP11-146N23.4 |
| CICP26 | AC067959.1 | NALCN-AS1 | RPEP4 | SLC9A3P1 | HDAC1P2 |
| RP5-908M14.5 | FAM204BP | AC097523.1 | AC016700.2 | TTLL7-IT1 | LINC01271 |
| RP11-5P18.5 | RPL23AP25 | LINC00892 | RP5-837O21.2 | MTND3P17 | RP1-67K17.3 |
| RP11-537A6.9 | SYF2P2 | HMGA1P8 | RPS24P6 | RP11-259F16.3 | RP11-536P6.3 |
| RP3-468B3.2 | KLF4P1 | RP11-324I22.2 | RP11-216N14.9 | CNOT7P1 | NPIPB7 |
| RP3-339A18.6 | RP11-445K13.2 | RP11-558F24.2 | TRGV2 | AC009960.3 | AC018878.3 |
| ZNF90P2 | RP11-307L3.4 | AC092664.1 | RP11-324F21.1 | RP4-569D19.5 | AC104809.4 |
| AP000688.29 | PRKAR1AP | RP11-296A18.5 | RP11-104L21.2 | AC012065.5 | KRT18P67 |
| CYP2AB1P | LINC01077 | AC125238.2 | U40455.1 | KRT18P62 | RP1-187B23.1 |
| RP3-467K16.2 | HNRNPA1P60 | OR5B1P | LINC01518 | HCG21 | RP1-122K4.2 |
| AC017104.2 | LINC00690 | KRT8P15 | RP4-635A23.3 | RP11-153K11.3 | BTF3P5 |
| RP11-62H7.2 | DDX11L10 | CYP2T1P | PGAM1P11 | RP5-905G11.3 | RP11-491H19.1 |
| OR52T1P | CICP1 | AC093388.3 | SPIN4-AS1 | AC083899.1 | HNRNPA1P27 |
| CEACAMP1 | RP11-312J18.7 | RPL22P11 | RP11-342M1.7 | AC012506.2 | LL22NC03-30E12.11 |
| LINC00284 | RPL36AP13 | AP001628.7 | AC004947.2 | MTCO1P20 | RP11-154P18.2 |
| AP001442.2 | AC139887.4 | AP000695.4 | RP11-535M15.2 | RP11-135A24.4 | AC017078.1 |
| ETF1P3 | AC093732.1 | AC104113.3 | AC022201.5 | AC026904.1 | RPL7P44 |
| RP11-182110.2 | MTCO2P17 | EZR-AS1 | LINC01503 | XXbac- | RPS15AP40 |
| | | | | BPG181B23.6 | |
| RP11-154D17.1 | AC004012.1 | LINC01360 | RP11-288L9.1 | RP11-310H4.2 | AC016735.3 |
| FDPSP2 | RP11-297H3.3 | RP11-379J5.5 | AC013439.4 | IGKV3OR2-268 | CTD-3105H18.11 |
| RP11-214N15.5 | RP11-236P24.3 | TXNP6 | RP11-254B13.4 | RP11-452D2.2 | FAM96AP1 |
| LL0XNC01-237H1.3 | LINC00463 | EDDM3CP | TPRG1-AS1 | RP3-388E23.2 | MTND4P11 |
| KRT19P4 | RP11-145A3.4 | RP11-288G3.4 | RPL13AP6 | RPL23AP83 | AC055764.1 |
| RP11-395L14.3 | TCEB2P1 | RP11-64P14.7 | RP11-730A19.9 | MTCYBP1 | AC004854.4 |
| RP5-887A10.1 | LINC01191 | U82671.8 | RP5-1050D4.2 | RP1-67A8.3 | FHP1 |
| RP4-704D21.2 | RPL7L1P1 | RP11-353N4.5 | RP11-430L16.1 | AC012370.3 | RP11-146I2.1 |
| RP1-138B7.4 | RPL30P2 | COX7BP2 | RP11-213H15.1 | RP13-16H11.7 | CLYBL-AS1 |
| RP11-432J24.3 | RPL9P2 | MRPS10P2 | BCAS2P2 | RPS26P47 | PFN1P3 |
| RPSAP31 | SNX18P7 | AC007278.3 | RP11-561O23.5 | ELK2BP | CCDC188 |
| RPL5P7 | CICP13 | RP11-213G2.1 | RP11-528G1.2 | RP11-666F17.1 | RP11-1275H24.1 |
| AC005534.6 | RP11-211N8.6 | RP11-266I3.1 | CLDN34 | RP11-496N12.6 | OR7E46P |
| RPL13AP20 | GS1-124K5.10 | FYTTD1P1 | C5orf58 | PPP1R2P1 | RP11-37L2.1 |
| RP11-100G15.7 | RP3-510D11.2 | LINC01309 | AC022431.3 | TPMTP2 | RP4-784A16.1 |
| FABP5P14 | AC010096.1 | RP11-5P18.1 | RPL7P11 | AC016894.1 | OR7E155P |
| AC104820.2 | ACTBP13 | NUS1P2 | AC073283.4 | GPR50-AS1 | RP11-225H22.4 |
| VDAC1P3 | PRUNEP1 | TUBB8P11 | CTB-107G13.1 | ATP5A1P8 | LL22NC03-123E1.5 |
| AP002381.2 | RP11-203H2.2 | LINC00412 | LINC01103 | RP11-483H20.4 | AC114730.7 |
| RFTN1P1 | FAM197Y2 | AC090505.5 | LINC01135 | U91328.2 | AC092687.5 |
| RP11-5P22.1 | LINC01239 | CDC42P2 | CTD-3105H18.7 | NDUFA5P3 | PIGFP2 |
| MTND5P26 | AC007879.2 | RP11-384C4.7 | AP000344.3 | RP11-101O6.2 | GRID1-AS1 |
| SDHCP3 | AC104667.3 | RP11-356I2.1 | LINC00700 | SHISA8 | RP11-534L20.4 |
| RP11-280O24.3 | XX-C2158C12.1 | CUBNP2 | AC013429.5 | AC016745.3 | SNRPCP19 |
| RP11-344B5.4 | AF121897.4 | RP11-109I13.2 | AP001626.2 | AC097468.7 | MNX1-AS2 |
| BMP7-AS1 | RP11-61I13.3 | AC098820.2 | RPL7P8 | LINC00940 | RP11-637O19.2 |
| VDAC1P4 | FNDC1-IT1 | RPL7L1P3 | WI2-85898F10.1 | RP11-1217F2.1 | RP11-83A16.1 |
| RP11-90L20.2 | AC128709.3 | AC013474.4 | COX6CP17 | RPSAP16 | RP5-857K21.2 |
| RCBTB2P1 | LINC01366 | RP11-348H3.4 | RP11-613C6.4 | AC009495.2 | RP1-3E10.2 |
| RP11-121A14.2 | TUBBP6 | RP11-236P24.1 | RP1-151B14.6 | DANT2 | RP11-384B12.2 |
| AC010240.2 | TMEM185AP1 | KDM5C-IT1 | FLJ41941 | AC137723.5 | MRPL53P1 |
| GXYLT1P6 | AC012360.4 | GEMIN2P2 | AC005301.8 | AC023128.2 | CTA-276O3.4 |
| RP11-399E6.1 | RPL36AP15 | DNM1P51 | RP4-764D2.1 | GS1-600G8.5 | EPN2-AS1 |
| RP4-641G12.4 | AC005808.3 | AC010149.4 | KATNBL1P3 | SUMO2P10 | RP11-342M3.5 |
| MTATP6P17 | DPY19L2P4 | XXbac- | RP5-928E24.2 | AC006946.15 | AC114755.7 |
| | | B476C20.14 | | | |
| LINC01221 | RP11-693N9.2 | LLPHP2 | AC009505.2 | MSC-AS1 | LINC00402 |
| RP11-230B22.1 | AC087499.5 | SUMO1P4 | NOP56P1 | AP000477.2 | RP3-412A9.12 |
| AC073150.6 | RP11-448G4.2 | LINC00494 | AC016644.1 | LINC00484 | LINC00298 |
| RP11-109P14.8 | AC018442.1 | RP11-395P16.1 | CYCSP52 | ANKRD34C | MFRP |
| AC013268.3 | AC007349.5 | TRIM60P19 | PHACTR2-AS1 | BRD7P5 | DPYD-AS2 |
| AC079779.5 | ZNRF3-IT1 | HCFC1-AS1 | MYL6P2 | ADAM21P1 | RP5-1022P6.5 |
| LINC00837 | TBCAP2 | BHLHE40-AS1 | CNN2P3 | AC124944.4 | HS6ST2-AS1 |
| OR7E145P | RP11-338C15.5 | RP1-102G20.4 | ARHGEF7-AS2 | RP11-59O6.3 | AC023115.2 |
| TSPEAR-AS1 | RP11-460E7.8 | MTCO2P11 | NEXN-AS1 | RP11-250H24.6 | AC007405.8 |
| AC008280.1 | AC002543.2 | EGOT | RP11-472G23.10 | VN2R19P | AC007050.17 |
| AC105921.5 | HIGD1AP12 | RP11-160E2.16 | PATE3 | RP11-323C15.2 | RP11-90O23.1 |
| AC019117.2 | CHIAP1 | FABP5P2 | AC073410.1 | GAPDHP14 | HSPB1P1 |
| RP5-101101.3 | HMGN2P28 | BNIP3P2 | RP11-630I5.1 | DUX4L26 | RP11-65J3.6 |
| RP11-231P20.2 | PRADC1P1 | GAPDHP57 | RP1-182D15.2 | SSU72P6 | RP11-163G10.4 |
| RP11-297K7.1 | PRKX-AS1 | RP11-105N14.3 | LINC01376 | PPP1R11P1 | RP11-645N11.3 |
| VEZF1P1 | AC017048.2 | MTCYBP24 | RP11-15J10.8 | DIAPH2-AS1 | AC017083.2 |
| LINC01361 | RP4-728D4.3 | CLEC2L | AC114737.3 | AC013463.2 | NPM1P8 |
| EEF1GP7 | RP11-340I6.7 | TLX1NB | OR7E109P | RP11-348H3.5 | AC019117.1 |
| SLFN14 | AC005300.5 | RP3-486I3.5 | RP11-759F5.1 | PPIAL4G | FMR1-IT1 |
| RP5-1103B4.3 | AC079584.2 | RP11-334A14.2 | RP11-440G9.1 | POU5F1P5 | AC084809.3 |
| RP11-320G24.1 | RP11-447M12.2 | RP11-132N15.2 | AC090018.1 | RP11-79M19.2 | RP11-107M16.2 |
| RP11-296A18.6 | UBE2L5P | ATG10-IT1 | AC067956.1 | AC130689.5 | LINC00427 |
| RP11-104D3.2 | AC007040.8 | RP11-89N17.4 | RP3-336K20_B.2 | KLF2P4 | NPM1P40 |
| AC007278.2 | RP1-125I3.2 | AC097711.1 | H3F3BP1 | RC3H1-IT1 | RP1-118J21.5 |
| AC079807.3 | RP11-243J16.7 | YWHAQP5 | RAD23BP1 | SNRPGP14 | RP4-669L17.2 |
| EEF1DP8 | IFNA4 | CTA-221G9.7 | DLX2-AS1 | RP11-108L7.4 | AC104076.2 |
| RP11-85O21.2 | RP11-296P7.4 | BZW1P1 | LINC01010 | SMAD9-IT1 | RP11-359I18.1 |
| RP11-411K7.1 | RP5-1184F4.5 | RP11-365O16.1 | AC078941.1 | BCRP8 | RP11-383C6.2 |
| RPS3AP12 | ANKRD20A7P | LINC01563 | AC091633.2 | RP11-239E10.2 | LINC01474 |
| AC105393.1 | RPL23AP23 | RP11-44H4.1 | RP3-522J7.5 | AP001615.9 | RPS20P5 |
| XXbac- | ASS1P7 | RP11-128B16.3 | RP5-1063M23.2 | RP4-651E10.4 | RP11-792A8.1 |
| B476C20.11 | | | | | |
| RP3-329E20.2 | AC003092.2 | RP11-98D18.7 | RP11-366M4.8 | ZDHHC20-IT1 | LINC01141 |
| OR52N3P | GAPDHP51 | ANKRD44-IT1 | LINC00614 | RP11-544A12.4 | YBX1P9 |
| KRT18P52 | RPL21P33 | RP1-315G1.1 | RP11-16C18.3 | RPL34P1 | AC097374.3 |
| RP13-436F16.1 | RP4-550H1.5 | RPL5P2 | RP11-168K11.2 | EHMT2-AS1 | RP11-73E6.2 |
| RP11-342M1.6 | AC018890.4 | TAAR9 | RP1-131F15.2 | CYP2F2P | LINC01056 |
| HAND2-AS1 | MRPS6P2 | DDX10P1 | DNAJC19P1 | MCFD2P1 | RP11-443F16.1 |
| RPL12P27 | RPS7P15 | AC019080.1 | RP11-229A12.2 | NR2F1-AS1 | DLGAP5P1 |
| ZBTB40-IT1 | IMPDH1P4 | SETP4 | RP11-322A17.1 | TRBV30 | MTND2P8 |
| RP4-568F9.3 | AC073069.2 | AC068492.1 | OR51R1P | RSL24D1P8 | RP11-96C23.9 |
| CKMT1B | HIGD1AP8 | RP4-809F4.1 | AP000563.2 | RP11-201O14.1 | RNF223 |
| XIAP-AS1 | SYP-AS1 | AC004461.4 | RP5-1065J22.2 | PATE4 | RP11-509J21.2 |
| RP11-334N17.1 | BRWD1-IT1 | RPL21P121 | RP11-165J3.5 | RP11-402L1.4 | RP11-139I14.2 |
| CAMTA1-IT1 | RP11-226L15.1 | AP001092.4 | XRCC6P1 | AC069257.6 | RP11-305L7.3 |
| RPSAP2 | RP1-159A19.4 | RP11-15P13.1 | RP1-127L4.10 | CDC27P2 | RPL23AP71 |
| CDK2AP2P2 | RP11-554F20.1 | AP000361.2 | RPL7P53 | XXbac- | RP13-926M18.1 |
| | | | | B135H6.15 | |
| OR2L9P | PKN2-AS1 | MTCO3P17 | AC096775.2 | PYY2 | GCSHP3 |
| RP11-383J24.2 | RP11-20J15.2 | IGKV1OR10-1 | AF064858.10 | OR9A1P | MTCO2P19 |
| FRY-AS1 | HMGB3P5 | HLCS-IT1 | RNASEH2CP1 | KRR1P1 | RPL30P4 |
| RP11-360O19.4 | IRGM | AP004289.2 | AC008937.2 | EEF1A1P28 | P4HA2-AS1 |
| DCTN1-AS1 | NPAP1P3 | AC002368.4 | RP3-423B22.5 | AC092620.3 | RP11-129B9.1 |
| DDR1-AS1 | RP11-54A4.2 | GFOD1-AS1 | AC010894.5 | CICP11 | HNRNPA1P39 |
| RP11-400K9.4 | RPS3AP29 | RPL23AP33 | RP11-332O19.2 | RP11-432N13.2 | RP1-67K17.4 |
| RP4-630A11.3 | AC097533.1 | POU5F1P4 | LINC00398 | NALT1 | RPL23AP32 |
| KLF7-IT1 | RP3-393E18.2 | AC097523.3 | Z95114.5 | PPIL1P1 | AC013267.1 |
| TMEM161BP1 | RP11-231N9.1 | FLG-AS1 | NPAP1P6 | COX6CP10 | RP4-641G12.3 |
| ZDHHC4P1 | AF011889.2 | RP1-128O3.6 | MRPL48P1 | PPP1R26P1 | AC006195.2 |
| LINC01625 | AC066692.3 | LINC01589 | SLC9A3P2 | BRWD1-AS1 | LINC01053 |
| RPS2P35 | AC017079.4 | RP11-656D10.6 | RP5-875H3.2 | RP11-69L16.4 | GS1-309P15.4 |
| HMGN2P17 | AC067940.1 | RP11-402L1.12 | LINC00707 | RP3-436N22.3 | AC012360.6 |
| HOMER2P2 | RP11-100G15.4 | RP11-431K24.1 | RN7SKP198 | SNORA81 | SNORD97 |
| RP11-23D24.2 | RNU1-13P | SNORA47 | RP11-536C10.1 | RPL21P106 | GAPDHP39 |
| RPL27P12 | RPS20P22 | RN7SL546P | RN7SL589P | RPSAP4 | RN7SL184P |
| RP11-400L8.2 | RP11-492E3.51 | RP11-407P2.1 | PCDHA13 | CTD-2301A4.1 | RP11-101K23.1 |
| CYP4F62P | RP11-330L19.2 | RN7SL569P | RPL7P38 | KLHL41 | RPS3AP41 |
| RP11-163E9.1 | RPS4XP18 | MARK2P8 | PLCH1-AS1 | RPL9P5 | POM121L7 |
| RPS14P8 | RP11-9D8.1 | GOLGA2P2Y | RP11-109N23.1 | RN7SL12P | SETP11 |
| RN7SL388P | OR1J4 | RP11-449H3.1 | RUVBL1-AS1 | HMGN1P7 | RP11-543D10.2 |
| MTND1P3 | MYADML | RPL9P31 | RP4-803A2.2 | CTD-3193K9.1 | RP11-665C16.1 |
| RP3-461F17.1 | RP11-6F2.3 | RN7SL692P | RN7SL688P | RN7SL672P | RP11-496B10.3 |
| ITIH4-AS1 | DENND6A-AS1 | RN7SL213P | RN7SL754P | RP11-767L7.1 | RPL23AP72 |
| IGKV1-6 | RN7SL225P | IGKV1-37 | RN7SL34P | RN7SL674P | RN7SL530P |
| RPS10P16 | AC015691.13 | LINC01471 | AP001625.4 | RP11-246A10.1 | ENPP7P2 |
| YBX1P3 | RPL7P24 | RP11-561B11.1 | OR9A3P | RPS3AP32 | RPL9P30 |
| BMS1P3 | RP11-641C17.4 | RN7SL146P | FABP5P11 | RP11-204K16.1 | RP11-758P17.3 |
| CPHL1P | RPS27P29 | RN7SL587P | RN7SL794P | B4GALT4-AS1 | RN7SL717P |
| RN7SL100P | RP11-841C19.1 | RP11-331F4.4 | AC017002.1 | RPL36AP45 | VPS26AP1 |
| CTD-2061E19.1 | RPL9P3 | KIR3DL2 | RPS26P34 | RP11-95I19.1 | RN7SL472P |
| RN7SL116P | RPL29P2 | RN7SL277P | UOX | RP5-1061H20.3 | RPL21P123 |
| CTD-2313F11.1 | RPL3P9 | RP1-184J9.2 | AC010153.3 | RP11-20L24.1 | RN7SL258P |
| RN7SL403P | C1QTNF9 | RP11-174O3.3 | RP11-555K12.2 | AC006539.2 | MYL6P4 |
| PNMA2 | RPS2P39 | RP11-430C7.4 | RPS4XP15 | KRBOX1 | ERLEC1P1 |
| AC098831.4 | RN7SL288P | RP11-435F17.1 | AC073934.6 | MTND4LP7 | CTD-3236F5.1 |
| AC093627.10 | RP11-572F4.1 | IGKV1-16 | RP11-65E22.2 | RN7SL209P | RP11-372E1.1 |
| HOXA11-AS | CTD-2036J7.1 | RPL29P24 | RN7SL709P | RP11-689J19.1 | RPL7P43 |
| RPL17P40 | RPL17P44 | FLJ46066 | PRICKLE2-AS1 | RPL22P19 | LINC00881 |
| ARHGAP31-AS1 | RP4-752I6.1 | RP11-106J23.1 | TDGF1 | RN7SL165P | SNAPC5P1 |
| RP11-572M11.1 | MTND4LP17 | RN7SL443P | RN7SL801P | RP11-275H4.1 | AC093620.5 |
| ENPP7P4 | RP11-221J22.2 | RPL34P33 | WDR82P2 | SETP6 | RN7SL385P |
| PMS2P11 | RP11-758P17.2 | RPL36AP43 | HNF1A-AS1 | RP11-192C21.1 | FAM195CP |
| RN7SL505P | EEF1A1P25 | RPL37P6 | CTD-2224J9.4 | TDGF1P6 | RPL7P19 |
| RN7SL182P | LINC00635 | PTPRG-AS1 | RP11-85G20.2 | CA15P1 | RN7SL767P |
| RP11-624D20.1 | ZBTB20-AS1 | IGKV2D-24 | RN7SL338P | PAQR9-AS1 | ATP5A1P7 |
| PRICKLE2-AS1 | RPL23AP8 | RN7SL482P | RP11-520D19.2 | CTD-2007H13.1 | KRTAP5-4 |
| RP11-34P13.9 | RP13-585F24.1 | UGT1A1 | RPS23P6 | INMT | RN7SL253P |
| RPS27P12 | RP11-732A19.1 | RN7SL704P | RN7SL265P | VWFP1 | ZNF90P1 |
| XACT | RP11-555M1.3 | RN7SL438P | RN7SL390P | ENO1P4 | RP11-1000B6.2 |
| RPL7P42 | RN7SL434P | RN7SL363P | KLF3P2 | RP11-85I21.1 | RP11-292E2.2 |
| HOGA1 | RN7SL517P | HNRNPA1P24 | CTC-340A15.2 | RN7SL76P | RN7SL566P |
| RN7SL323P | RP11-338L18.1 | ALG1L15P | RN7SL68P | RP11-1396O13.1 | RP11-70F11.2 |
| RP11-690C23.2 | DNAJB8-AS1 | RPS3AP49 | CTD-2555K7.1 | MARK2P6 | TOPORSLP1 |
| SERBP1P3 | ABCF2P1 | ARHGDIG | RN7SL127P | RP11-568K15.1 | RN7SL594P |
| RN7SL306P | RN7SL57P | LINC00996 | RP11-659G9.1 | RP11-197K3.1 | RPL12P32 |
| RP11-875H7.5 | CTD-2184D3.1 | RN7SL683P | RPL6P9 | RP4-631H13.2 | RN7SL800P |
| RP11-267J23.1 | C3orf67-AS1 | RP11-731J8.1 | UPK3BP1 | RPS15AP32 | RN7SL500P |
| RP11-270M14.1 | RP3-477O4.5 | NDUFB4P1 | KLHL6-AS1 | OR7E100P | MRPL42P4 |
| RN7SL144P | RPL21P11 | RP11-469J4.3 | CSAG4 | CTD-2339M3.1 | RPS3AP34 |
| CTC-359D24.3 | RPS24P16 | RP11-271C24.2 | RP11-745A24.2 | RN7SL40P | CCT4P1 |
| CTD-2015H6.2 | RN7SL362P | RN7SL806P | RP11-562A8.1 | RP11-30K9.1 | RHOT1P3 |
| RN7SL824P | IGKV1D-17 | RP11-651P23.5 | RP11-436H11.1 | RPS26P21 | KRT8P35 |
| RN7SL749P | CTC-484M2.1 | RP11-190P13.2 | RPL5P11 | RN7SL413P | RP11-298O21.6 |
| RPL12P7 | EPS15P1 | RPSAP3 | FTH1P23 | RP11-446F17.3 | RPL7P40 |
| FTH1P4 | RP11-697H10.1 | RN7SL239P | RN7SL16P | RPL12P35 | RP11-556N21.1 |
| PTMAP8 | MARK3P3 | RN7SL354P | GAPDHP47 | RP11-58E21.1 | RBMY2FP |
| RPL31P58 | RN7SL842P | RP11-613M5.2 | LINC00870 | RPS3P7 | RN7SL452P |
| RN7SL22P | RP11-115N4.1 | RP11-734J24.1 | HNRNPA1P17 | RP11-286H14.8 | AC073130.3 |
| RN7SL350P | RN7SL558P | RN7SL614P | CTGLF9P | RPL31P61 | RP11-268I9.1 |
| POM121L6P | RP11-397E7.1 | RN7SL285P | RP11-538P18.1 | FGF14-IT1 | RN7SL174P |
| RN7SL307P | RP11-445N18.7 | RP11-57G22.1 | RN7SL775P | CTD-2110K23.1 | AC012442.5 |
| RP11-454H13.1 | RP11-473O4.1 | RP11-330L19.1 | RPL35AP32 | RP11-245J9.4 | RPL23AP49 |
| RP5-837J1.1 | AF165138.7 | RP11-715J22.1 | RPS18P13 | INGX | RPL7P51 |
| RP11-572M11.2 | RN7SL337P | RP11-521D12.5 | UBA52P5 | RPS6P21 | RPS23P1 |
| RP11-775J23.2 | RP11-627K11.1 | IGLVIVOR22-2 | RN7SL19P | CTC-458A3.1 | RN7SL649P |
| RN7SL364P | RN7SL838P | UPK3B | RP11-2I17.1 | RP11-14I2.2 | RPL35AP26 |
| RPS2P44 | RP11-281P11.1 | RP11-215P8.1 | RP11-4K3_A.3 | WBP1LP1 | RPS4XP14 |
| RPS3AP40 | OR7E66P | RP11-379F4.1 | RN7SL598P | RN7SL638P | RN7SL105P |
| RPL23AP63 | RPLP0P2 | CTD-3131K8.1 | RP11-864G5.1 | RP11-112N19.1 | OR7E89P |
| RP11-390K5.1 | RN7SL610P | RN7SL67P | RN7SL487P | RN7SL419P | RN7SL320P |
| ELFN2 | RPSAP5 | RN7SL430P | RPS12P21 | RN7SL743P | RN7SL684P |
| RP11-473P24.2 | RPS24P17 | RN7SL447P | RP1-300I2.2 | RP11-1396O13.2 | B3GAT3P1 |
| RPL35P6 | RP11-266K4.1 | RPL5P24 | RP11-389C8.1 | MARK2P17 | GSTA2 |
| RN7SL833P | RPSAP51 | RP11-757F18.3 | RP11-1070A24.1 | RP11-810P12.1 | RP11-114H7.1 |
| RP11-379K17.5 | FOXP1-AS1 | PDZK1P1 | ZFAND6P1 | RPS4XP10 | LINC01330 |
| RPL26P35 | RN7SL698P | GMCL1P1 | IFITM10 | RP11-139K4.2 | RP11-64K12.1 |
| RN7SL168P | RN7SL395P | RN7SL36P | RN7SL845P | CTB-47B8.1 | RP11-119K6.6 |
| RP11-654C22.2 | RP11-531F16.3 | LY6G6D | RN7SL145P | RN7SL330P | RP11-428G5.1 |
| IGKV3-15 | RP11-354H21.1 | RP11-206M11.7 | ERVFRD-1 | OR2A1-AS1 | RP11-278L15.6 |
| LINC01391 | WNT5A-AS1 | RP11-713H12.1 | RN7SL334P | RP11-241J12.1 | RN7SL863P |
| XKRY2 | RN7SL280P | RPL10AP1 | CTAGE8 | PTCHD4 | RP11-133K1.1 |
| RN7SL47P | ASLP1 | RP13-1056D16.2 | RP11-373E16.3 | RN7SL153P | ALKBH3-AS1 |
| RP11-529F4.1 | CTD-3064M3.4 | RP11-744N12.3 | RP11-286E11.1 | ARNTL2-AS1 | KB-1471A8.1 |
| AC004053.1 | RP11-847H18.2 | RP11-620J15.2 | MIR4300HG | CEBPA | GS1-24F4.2 |
| LINC01513 | ALDH1L1-AS2 | RP11-260E18.1 | RP11-77I22.2 | RP11-13A1.1 | RP11-386I8.6 |
| LINC00968 | RP11-7F17.5 | RP11-1094H24.4 | LINC00535 | RASSF8-AS1 | RGMB-AS1 |
| SOCS2-AS1 | RP11-700H6.1 | CTC-338M12.7 | RP11-588G21.2 | LINC01498 | RP11-273G15.2 |
| CTD-2235C13.2 | RP11-392P7.6 | MIR4458HG | CTB-127C13.1 | RP3-510L9.1 | RP11-543C4.1 |
| RP11-422N16.3 | DPH6-AS1 | INSL3 | RP11-709A23.1 | CTC-338M12.9 | SMURF2P1 |
| CTD-2012J19.2 | RP11-472K22.1 | GAPDHP60 | ATG10-AS1 | RP11-153M7.1 | KCTD9P5 |
| CTD-2139B15.2 | RP11-366M4.12 | AC037459.4 | CTB-49A3.2 | CTC-499J9.1 | CTD-2194F4.2 |
| TNXA | DDX43P1 | CTD-2308B18.3 | RP11-717H13.1 | RP11-293A21.2 | LINC00504 |
| RP11-501M7.1 | FOSL1P1 | RP11-503N18.4 | RP11-576N17.3 | PRR5-ARHGAP8 | KB-1269D1.8 |
| GAPDHP61 | RP11-1191J2.4 | RP11-284A20.3 | RP11-321E2.3 | RP4-598P13.1 | CTD-2207A17.1 |
| RP11-640B6.1 | DDX11L9 | CTC-338M12.2 | ZFAT-AS1 | LNX1-AS2 | RP11-145G20.1 |
| CTC-338M12.1 | RP11-265O12.1 | RP11-174F8.1 | RP11-10A14.5 | RP11-241F15.1 | RP11-90P5.1 |
| CTD-2516K3.3 | RP11-158I23.1 | RP11-366M4.6 | RP11-302F12.5 | LINC00992 | TOX4P1 |
| CTC-493L21.2 | RP11-377G16.2 | CTC-236F12.4 | NMNAT1P4 | ACTN3 | CTC-276P9.2 |
| RP11-974F13.5 | LINC01095 | ZNF807 | CTB-17911.2 | RP11-227H4.5 | CTD-2016O11.1 |
| TNFSF12-TNFSF13 | CTC-537E7.3 | RP11-950K24.2 | FAM138E | RP11-72K17.2 | HMGB1P44 |
| BCLAF1P1 | CTD-2256P15.1 | CTD-2113L7.1 | RP11-395I6.1 | LAMTOR3P2 | RP11-87F15.2 |
| RP11-815N9.2 | RP11-317B7.2 | CTNNAP1 | SUMO2P6 | CTB-113P19.1 | COX6B1P5 |
| TBCAP3 | RP11-326I11.1 | RP11-72L22.1 | KRT18P25 | PCNAP1 | LINC01033 |
| WSPAR | RP11-478C1.8 | C5orf66-AS1 | AC051649.12 | RP11-381N20.2 | CTB-35F21.2 |
| SLED1 | PRDX2P3 | CTD-2290P7.1 | AC006445.7 | RP11-1C1.5 | RP11-1C1.6 |
| RP11-304F15.5 | CTC-506B8.1 | CTD-3080P12.3 | AC138517.4 | ATP5L2 | CDH12P2 |
| RP11-452J21.2 | BCL9P1 | RP11-557J10.3 | PGAM1P8 | RP11-468H14.2 | MTHFD2P6 |
| UNC93B8 | CTC-436P18.3 | ACTR3BP4 | FTH1P24 | AC010468.2 | ATP6V1G1P6 |
| RP11-834C11.6 | RP11-1C1.4 | HMGB3P17 | CTD-2234B20.1 | RP11-317O24.1 | IL20RB-AS1 |
| RP11-391J13.1 | CTC-563A5.2 | RP11-438D8.2 | CFAP99 | CTD-2224J9.7 | KRT18P45 |
| LINC01091 | CTB-49A3.5 | LINC01470 | GAPDHP70 | EGFLAM-AS1 | ZEB2P1 |
| LINC01438 | RP11-642E20.3 | RP11-789L4.1 | HSPD1P15 | AC226118.1 | CLRN2 |
| CTB-46B19.2 | RP11-703G6.1 | OR52V1P | RP11-710F7.3 | HOXC13-AS | RP11-552M14.1 |
| OR7E94P | C5orf66-AS2 | VN1R104P | TNRC18P1 | CTD-2227C6.2 | MIR143HG |
| RP11-217C7.1 | TUBA3GP | KRT19P3 | RP11-1336O20.2 | RP11-598F7.4 | RP11-217E13.1 |
| RP11-60A8.1 | RP11-563M4.1 | RP11-447H19.3 | SCARNA22 | CTC-428G20.2 | PGBD4P3 |
| GAPDHP76 | RP11-166J22.1 | HS3ST5 | RP11-983C2.2 | CTB-57H20.1 | RP11-434D9.2 |
| RP5-1029K10.2 | LINC01265 | CTD-2280E9.1 | XXbac- | UQCRBP3 | CCDC79 |
| | | | B444P24.8 | | |
| YWHAQP6 | TMEM158 | RP11-814P5.1 | RP11-584P21.4 | RAF1P1 | CTD-2003C8.2 |
| CBX3P3 | RP11-148B6.2 | RARRES2P4 | RP11-576N17.5 | CACNA1G-AS1 | AC107982.4 |
| RP11-553P9.1 | MORF4L2P1 | CTC-498M16.2 | RP11-268P4.5 | CTC-321K16.1 | HMGN1P15 |
| RP13-577H12.3 | RP11-397E7.2 | SEPT14P9 | CIR1P2 | RP11-9G1.3 | SEPHS2P1 |
| LINC01612 | RP11-122A21.2 | RP11-94C24.8 | NCAPGP1 | ZBED1P1 | RP11-44F21.4 |
| MGC32805 | KDELC1P1 | LINC01340 | CTD-2272G21.2 | RP11-503L19.1 | AC008592.5 |
| RP11-22A3.2 | TRIM75P | CTC-467M3.3 | UNC93B4 | RP11-395P13.4 | KLHL2P1 |
| RP11-448G15.1 | CTC-461F20.1 | SEC63P2 | AACSP1 | KIAA1210 | RP5-877J2.1 |
| RP11-834C11.5 | CLSTN2-AS1 | TRIM36-IT1 | DUTP7 | ENPP7P9 | PPM1AP1 |
| FLJ33360 | RP11-640N11.2 | ABT1P1 | AC007126.1 | AC008984.6 | GPR162 |
| ABHD17AP3 | RP11-254I22.1 | KNOP1P5 | RP11-215P8.4 | LINC01511 | RP11-39E3.3 |
| RP11-517I3.1 | RP11-597D13.8 | RP11-933H2.4 | XXbac- | RP11-295K3.1 | RP11-834C11.7 |
| | | | BPG32J3.19 | | |
| HNRNPKP5 | LINC01596 | RP11-6L6.2 | CTB-161M19.1 | KB-1042C11.1 | RP11-392B6.1 |
| RP11-366M4.14 | C8orf17 | RP11-404E16.1 | RP11-834C11.4 | RP11-232L2.1 | RP11-451H23.1 |
| RP11-241F15.10 | RP11-440I14.4 | AC138035.1 | RP11-1084J3.3 | RP5-1063M23.1 | RP11-63A11.1 |
| RP11-723O4.3 | RP11-531A21.4 | DCAF13P2 | RP11-91H12.4 | HNRNPA3P13 | RP11-11N5.1 |
| HMGB1P29 | METTL21EP | CTD-2281M20.1 | RP11-588L15.2 | RP11-632F7.3 | CTB-161M19.2 |
| LNX1-AS1 | ENPP7P11 | RP11-402J6.3 | AC073635.5 | RP13-128O4.3 | ORAOV1P1 |
| RP11-159K7.1 | SLC25A30-AS1 | RP11-629B11.4 | HMMR-AS1 | WBP1LP4 | CTC-321K16.4 |
| ZNF969P | RP11-168A11.4 | RP11-115D19.1 | FCF1P8 | RP11-701P16.2 | RP11-701P16.3 |
| RP11-478C1.7 | MTND5P5 | HOXC-AS3 | RP11-45L9.1 | OR7E21P | RP11-101E3.5 |
| RP11-689P11.3 | LINC00589 | RP1-68D18.2 | RP11-54F2.1 | RP11-400D2.2 | CTC-278L1.1 |
| CTD-2187J20.1 | RP11-889L3.4 | RP11-1057B8.2 | CNOT10-AS1 | MIR3945HG | RP11-540D14.8 |
| RFPL4B | RP11-340A13.2 | RP11-400D2.3 | RP11-506H20.1 | CTD-2337A12.1 | RP11-317G22.2 |
| AC006499.1 | RP11-461G12.2 | HSPD1P11 | MTND3P5 | RP11-697N18.1 | RAB5CP2 |
| AP000350.10 | RP11-18H21.3 | AC096582.9 | RP11-484O2.1 | YTHDF1P1 | RP1-240K6.3 |
| LINC01256 | FAM90A25P | AC008592.4 | CTC-369A16.2 | RTEL1P1 | LINC01094 |
| HAUS1P1 | RP11-436H11.5 | RP11-46A10.6 | IMPA1P | RP11-381N20.1 | CTD-2245E15.3 |
| LINC00605 | RP11-478C6.1 | CTC-313D10.1 | COQ10BP2 | HMGB1P35 | LINC01267 |
| NIFKP7 | HADHAP1 | RP11-673E1.1 | RP11-610P16.1 | AC009487.5 | RP11-826N14.4 |
| UNC93B7 | RP11-774D14.1 | RP11-119D9.1 | RP11-20I20.1 | PRB1 | AC007036.5 |
| RP11-614F17.2 | RP11-631M6.3 | RP11-181K12.2 | RP11-478C6.5 | RP11-618I10.4 | PTX4 |
| ACA59 | RNA5SP216 | RN7SKP179 | RNU4ATAC18P | SCARNA17 | RNU2-27P |
| SNORA31 | RN7SKP265 | YRNA | SCARNA16 | SNORA21 | RN7SKP272 |
| SCARNA6 | RN7SKP2 | SCARNA1 | SCARNA11 | RN7SKP64 | CTD-2373N4.5 |
| IGLV2-34 | RP11-459E5.1 | CTD-3023L14.2 | CTC-436K13.2 | RP11-589C21.2 | CIR1P1 |
| CTB-47B11.1 | RP11-279L11.1 | CTC-209H22.3 | RP11-152C15.1 | RP11-1149M3.2 | RP11-410L14.1 |
| HOXA10-AS | AC084082.3 | RP11-351A11.1 | RP11-359P18.6 | RP11-706C16.7 | RP11-546B8.3 |
| PGAM5P1 | NRBF2P4 | IGHV3-76 | C8orf88 | RP11-10A14.6 | IGKV2-14 |
| RP11-128L5.1 | RP11-99A14.1 | RP11-238K6.1 | MIOXP1 | RP11-779O18.1 | CTB-160O22.1 |
| AC008703.1 | SERPINE3 | IGHVIII-76-1 | RP11-61G23.2 | RP11-497H16.8 | RP13-923O23.6 |
| RP11-420B22.1 | CTD-3023L14.3 | KB-1460A1.2 | RP11-90P5.2 | RP11-731F5.2 | RP11-589F5.4 |
| KB-1254G8.1 | AC078852.2 | RP11-231D20.2 | CTB-47B8.4 | NCRNA00250 | CDC42P5 |
| AC133633.2 | RP11-619L12.4 | IGLV3-6 | IGLV2-28 | CTD-2363C16.2 | IGKV2OR22-3 |
| IGKV1-35 | RP11-110G21.2 | RP11-1C8.4 | IGHVII-26-2 | ATP6V1G1P2 | CTA-398F10.1 |
| RP1-170O19.14 | RP11-417F21.2 | XRCC6P4 | AC106801.1 | CTD-2114J12.1 | RP11-347C18.4 |
| RP11-1174L13.2 | RP11-402L5.1 | PYDC2 | CA3-AS1 | RP11-115C21.4 | CTD-2340D6.2 |
| KB-1184D12.1 | RP11-723D22.2 | IGHV3-30-2 | RN7SL260P | RP11-369K17.1 | AC144568.4 |
| CTC-370J7.1 | RP11-486M23.1 | IGLVV-58 | LINCR-0001 | CTD-2311A18.1 | SOD1P3 |
| NPM1P52 | KB-1615E4.2 | IGHVII-78-1 | KB-1410C5.2 | CTC-348L5.1 | RP11-567J20.2 |
| KBTBD11-OT1 | RP11-1259L22.1 | RP11-51J9.4 | RP11-787D18.1 | RP11-10N23.4 | RP11-893F2.13 |
| LZTS1-AS1 | LINC01289 | RP11-779O18.3 | KB-1460A1.3 | MARK2P11 | RP11-350F16.1 |
| RP11-527N22.2 | CYP4F44P | RP11-21C4.4 | RP11-439C15.4 | HMGB1P23 | LINC01030 |
| CTD-2363C16.1 | RP11-1134I14.6 | IGLV1-41 | KB-173C10.2 | RP11-96A1.5 | KB-173C10.1 |
| IGLVIV-66-1 | RP11-16P20.3 | IGLVI-38 | RP11-770E5.2 | RP11-380I10.3 | IGLV3-31 |
| CTB-113P19.3 | IGLVIV-53 | HIGD1AP18 | CTB-43E15.1 | RP11-363L24.3 | CTB-178M22.2 |
| LINC00051 | MAP2K1P1 | RP11-10J21.3 | BRIX1P1 | CTC-436K13.4 | RP11-328K2.1 |
| RP11-172E10.1 | RP11-697N18.2 | IGLVV-66 | IGLV3-7 | AC078852.1 | AC005740.5 |
| VENTXP6 | RBPMS-AS1 | CTB-7E3.1 | RP11-1007J8.1 | RP11-661A12.4 | RP11-359P18.5 |
| IGKV2-18 | RP11-1149M10.1 | AB015752.3 | RP11-419L20.2 | RP11-167P20.1 | CTC-558O2.2 |
| KB-1043D8.6 | RP11-11C20.1 | FAM90A12P | RP11-582J16.3 | RP11-1080G15.2 | RP11-17A4.2 |
| RP11-398H6.1 | RP11-18A15.1 | RP11-473O4.4 | RP11-131N11.4 | RP11-382J24.2 | RP11-6I2.3 |
| IGHV3-32 | CTB-17P3.4 | IGLVIV-59 | CTD-2045M21.1 | RP11-267M23.3 | RP11-318K15.2 |
| IGHVII-60-1 | MAFA-AS1 | RP11-10A14.3 | RP11-760H22.2 | RP11-578O24.2 | IGLVI-68 |
| RP11-211C9.1 | IGHV4-55 | RP11-56F10.3 | RP11-578F21.2 | RP11-179A10.1 | RP11-215D10.1 |
| RP11-182J1.1 | SIGLEC14 | RP11-347E10.1 | RP11-21L19.1 | RP11-864G5.3 | RP11-33I11.2 |
| RP11-231N3.1 | RP11-718B12.5 | RP11-304C12.3 | HSPB2-C11orf52 | OR7E11P | ALG9-IT1 |
| RP11-867G23.4 | RP11-867G23.13 | PRKRIRP4 | RP11-885L14.1 | SLC25A1P1 | RP11-23F23.2 |
| PTP4A2P2 | CTA-797E19.1 | KB-1073A2.1 | AP000487.4 | RANP3 | FBXO3-AS1 |
| RP11-867G23.10 | RP11-775A1.2 | RP11-113K21.1 | RP11-624D11.2 | RP11-463D19.1 | NAV2-AS3 |
| RP11-113D6.9 | OMP | RP11-196E1.3 | MTND2P26 | UBTFL9 | RP11-1007G5.2 |
| RP5-1173A5.1 | AP000662.4 | RP11-22P4.2 | RP11-700F16.2 | RP11-356J5.4 | RP11-702F3.1 |
| CTD-2560E9.3 | STT3A-AS1 | RP11-109L13.5 | RP11-265D17.2 | RP11-354A14.1 | COPS8P3 |
| RP11-659G9.3 | RP11-12D16.2 | RP11-565P22.6 | RP11-35J10.4 | RP1-145M24.1 | RP11-560B16.5 |
| RP11-351I24.3 | OR7E10P | RP11-801G16.2 | RP11-691N7.6 | RP11-1H15.1 | RP11-50B3.1 |
| RP11-27P7.1 | AC084121.17 | AC022182.1 | GVINP2 | FDPSP4 | AC022182.3 |
| RP11-672A2.4 | RP3-400B16.4 | CTD-2530H12.2 | SLC22A18AS | AF131215.6 | RP11-648O15.1 |
| RP11-203M5.2 | CTD-2523D13.2 | RP11-867G23.1 | RP11-113D6.3 | RP11-945A11.2 | CTD-2516F10.4 |
| PTPRJ-AS1 | NAV2-AS1 | RP11-482L11.1 | RP11-513D5.2 | RP11-263C24.3 | RP5-1160K1.8 |
| INMT-FAM188B | RP11-1081L13.4 | RP11-65M17.3 | RP11-167J8.3 | RP11-429J17.7 | RP11-702H23.2 |
| ALG1L9P | FTLP6 | CTD-2509G16.2 | RP1-68D18.3 | AP000442.4 | RP11-113K21.3 |
| ARL6IP1P3 | RP11-665E10.1 | RP11-45A12.2 | RP11-23J9.4 | RP11-347H15.5 | RP11-661A12.9 |
| RP1-317E23.6 | RP11-263C24.1 | RP11-843A23.1 | AF186192.5 | RP11-326C3.10 | RP11-58K22.4 |
| RP11-794P6.2 | RP11-406D1.2 | TBC1D26 | RP11-159H10.1 | AP006621.8 | RP11-91P24.3 |
| RP11-655M14.12 | OVOL1-AS1 | RP11-685M7.5 | GLTPP1 | HNRNPA1P76 | CTD-2313N18.8 |
| HSPE1P18 | AP002954.3 | RP11-720D4.3 | NANOGP8 | RP11-945A11.1 | RP4-541C22.5 |
| RP11-794P6.3 | OR5M11 | RP11-304M2.3 | RP11-438N5.4 | CTD-2655K5.1 | CTD-2523D13.1 |
| PPP1R1AP1 | CTD-322413.3 | RP11-430H10.2 | RP11-702F3.4 | RRM1-AS1 | ABCC6P2 |
| RP11-91I20.1 | RP11-757C15.4 | RP11-655C2.3 | RP11-624G17.3 | RP5-901A4.1 | OR52B2 |
| RP11-428C19.4 | RP11-160H12.2 | RP11-91I20.4 | CTD-2530H12.4 | RP11-484D2.5 | RP11-299M14.2 |
| CTD-233717.1 | NOX5 | RP11-700F16.3 | RP11-382M14.1 | CCDC179 | RP11-412B14.2 |
| RP11-619A14.3 | RP11-672A2.5 | RP11-1134I14.8 | RP13-726E6.2 | DUXAP5 | CTD-2028E8.1 |
| C8orf49 | HCAR3 | RP11-770G2.5 | PCDHA3 | CASC23 | CTD-2011F17.2 |
| AP002954.4 | CTD-2530H12.8 | RP11-510I21.1 | RP11-632K5.2 | CTD-2210P24.6 | SEPT14P8 |
| RP11-113D6.6 | RP11-998D10.1 | RP11-687M24.4 | RP11-1H15.2 | RP11-867O8.5 | RP11-65M17.1 |
| CTD-2381F24.1 | RP11-347H15.2 | AC005363.9 | RP11-164N3.1 | RP11-390K5.3 | RP11-780O24.2 |
| OR10V2P | CTA-797E19.2 | RP11-727A23.8 | RP11-627G23.1 | RP11-770G2.2 | UNC93B6 |
| RP11-273B20.3 | AP000462.2 | RP11-377D9.3 | FAM222A-AS1 | RP11-885B4.2 | RP1-127H14.3 |
| APOOP3 | RP11-46H11.2 | RP11-529H2.2 | RP11-283G6.5 | RP11-22B23.2 | LINC01479 |
| RMST | OR6J1 | RP5-1154L15.1 | RP4-559A3.7 | RP11-338K17.8 | Metazoa_SRP |
| RP11-612B6.1 | AC073610.5 | CTD-2102P23.1 | RP11-286N22.10 | RP11-351O2.1 | AP003419.16 |
| RP11-656E20.5 | RP11-69M1.3 | RP11-513G19.1 | PSMC1P9 | RP11-20D14.4 | AC084117.3 |
| TAS2R63P | TCEB1P31 | RP11-190A12.7 | PAPPA-AS1 | RP11-982M15.6 | RP11-709A23.2 |
| RP11-81H14.3 | AP000721.4 | RP13-672B3.5 | LINC01152 | RP5-944M2.4 | EGLN3P1 |
| RP11-968O1.5 | RP11-76C10.5 | RP11-820K3.4 | SMLR1 | RP11-627K11.3 | TSPAN9-IT1 |
| RP11-439H13.2 | AC005255.3 | RP11-1018J11.1 | RP11-1038A11.2 | RP11-771K4.1 | RPL7AP3 |
| RP11-989F5.1 | RP11-504G3.4 | METTL8P1 | RP11-338E21.3 | ABCC6P1 | RP11-21A7A.3 |
| RP1-7G5.5 | NTAN1P3 | RP11-349I1.2 | RP11-173P15.3 | RP11-428G5.4 | PPIAL4D |
| RP11-1060J15.4 | RP11-500M8.4 | RP1-102E24.8 | RP11-794G24.1 | RP11-809N8.4 | RP11-363J17.1 |
| RP11-428G5.6 | RP11-860B13.3 | RP11-212D19.5 | RP11-598F7.6 | NANOGNBP2 | ENPP7P5 |
| CYP2B7P | RP11-815J4.6 | MTRNR2L1 | RP11-76I14.1 | RP11-144023.8 | RP11-218M22.2 |
| RP11-599J14.2 | RP11-444B24.2 | RP11-785H5.2 | PGA5 | CACNA1C-IT3 | RP11-613F22.7 |
| RP11-45F15.1 | RP11-17G12.3 | RP11-234B24.5 | STH | RP11-503G7.1 | RP13-977J11.5 |
| RP11-76C10.2 | RP11-158L12.5 | TPT1P12 | AC138744.2 | GS1-410F4.5 | RP11-467L13.4 |
| CCND2P1 | RP11-664D1.1 | RP11-503G7.2 | IQSEC3P1 | RP11-817J15.2 | RP11-598F7.3 |
| RP11-6B19.2 | LIMS3 | RP11-972L6.2 | RP11-351O2.2 | RP11-800A3.7 | RP11-137N23.1 |
| RP11-783K16.10 | RP11-667M19.4 | RPL29P33 | RPEP6 | AE000658.22 | CLIP1-AS1 |
| LRRC37A13P | RP11-259O18.4 | OR4F28P | RP11-129B9.2 | RP11-320P7.2 | RP11-813P10.1 |
| CTD-2213F21.3 | RP11-58A17.3 | RP11-754I20.3 | RP11-611O2.5 | HNRNPA1P50 | FAM205BP |
| RP11-54A9.1 | RP11-136F16.2 | RP11-611E13.3 | RP3-405J10.4 | RP11-767I20.1 | RP11-434H14.1 |
| RP11-190J23.1 | RP11-557F20.2 | RP11-496I2.2 | RP11-511B23.2 | RP11-478B9.3 | MGAM |
| RP11-616L12.4 | BNIP3P6 | RP11-780K2.1 | RP11-74K11.1 | RP11-328C8.2 | RP11-644F5.12 |
| RP11-56G10.2 | RP11-2H8.2 | RP11-603J24.9 | RP11-263K4.3 | RP1-71H24.1 | RP11-290L1.3 |
| RP11-159D23.2 | RP11-359M6.1 | RP11-983P16.2 | MRPL2P1 | RP3-521E19.2 | RP11-956E11.1 |
| NPIPB1P | RP11-20E24.1 | RP3-405J10.2 | RP11-144F15.1 | RP11-44N21.1 | RP11-863H1.1 |
| GSTP1P1 | RP11-44N21.4 | RP1-128M12.3 | RP11-570L15.2 | RP11-278C7.2 | RP11-579D7.2 |
| CBX3P5 | RP11-76E16.2 | RP11-175P13.2 | ILF2P2 | MGAM2 | RP11-536C10.21 |
| RP11-186F10.2 | OR5BK1P | RP11-1143G9.4 | RP11-162P23.2 | CTB-193M12.1 | RP11-2H8.4 |
| RP11-46I1.2 | RP11-616L12.1 | RP11-263K4.1 | RP11-769N19.2 | RP3-432I18.1 | RP11-493L12.5 |
| RP11-493L12.3 | RP11-46I1.1 | RP11-620J15.1 | RP1-228P16.5 | RP1-228P16.4 | RP11-61E11.2 |
| RP11-603K19.1 | RP11-406H4.1 | Metazoa_SRP | CTD-2021H9.3 | RP11-474P2.2 | RP11-753H16.3 |
| RP11-18O15.1 | RP11-1105G2.4 | RP11-394J1.2 | NKX2-2-AS1 | ZNF75BP | RP11-654D12.2 |
| CLEC5A | RP1-46F2.3 | RP11-219B4.5 | RP11-570L15.1 | RP11-887P2.5 | POLR2KP1 |
| TESC-AS1 | BRWD1P2 | NUTF2P2 | RP11-690J15.1 | CTD-2314B22.1 | KLF17P1 |
| RP1-267L14.3 | RP11-161H23.9 | RP11-115H15.2 | RP11-536G4.2 | RP11-603J24.6 | RP11-651L5.2 |
| SLC25A3P2 | RP11-1070N10.4 | RP11-831F12.2 | RP11-326E7.1 | NT5CP2 | RP11-665C16.6 |
| RP11-711D18.2 | RP11-661G16.1 | RP11-109N23.5 | RP11-903H12.3 | RP11-361H10.3 | SPESP1 |
| SRMP2 | ZFP64P1 | DIO3OS | CTD-2062F14.2 | EIF3LP1 | RP5-892G5.2 |
| RP11-638I2.9 | RP11-16B13.1 | RP11-159L20.2 | CTD-2376120.1 | CTD-2313J17.3 | BLZF2P |
| DUX4L16 | RP11-320M16.2 | FAM181A-AS1 | NBEAP1 | CTD-3051D23.4 | SERPINA2 |
| LINC-ROR | COX7A2P1 | RP11-492D6.3 | RP11-354B3.1 | RP11-857B24.1 | RPL21P5 |
| LINC00930 | RP11-509A17.3 | RP11-517O13.3 | RP11-638I2.8 | RP11-407N17.2 | RP11-74M13.4 |
| LINC01269 | RP11-1033H12.1 | RP11-433J8.1 | LINC01193 | CKAP2P1 | RP11-404P21.6 |
| RP11-603B24.1 | LINC00637 | RP11-982M15.7 | RP11-862G15.1 | RP11-80A15.1 | RP11-688G15.3 |
| RP11-841O20.2 | RP11-613G13.1 | DIO2-AS1 | NANOGP7 | BMS1P15 | KRT18P6 |
| RP11-404P21.9 | CTD-2302E22.2 | RP11-7F17.3 | CTD-2555K7.3 | CTD-2566J3.1 | RP11-355I22.5 |
| RP11-286O18.1 | RP11-945F5.1 | RP11-894P9.2 | RP11-463J10.4 | CTC-554D6.1 | LINC01146 |
| EIF4EBP1P1 | FDPSP3 | RP11-270M14.5 | HIGD1AP17 | RP11-193F5.1 | SCOCP1 |
| RP11-260M19.2 | BHLHB9P1 | NPM1P5 | RP11-47I22.1 | RP11-463J10.2 | RP11-1078H9.2 |
| RP11-255G12.2 | RP11-857B24.5 | SALRNA1 | RP11-747H7.1 | RP11-829H16.5 | RP11-934B9.3 |
| LINC01467 | COX5AP2 | RP11-125H8.1 | RP11-368P15.2 | RP11-398E10.1 | DOCK11P1 |
| RP11-671J11.7 | RP11-8L8.2 | RP3-449M8.6 | RP11-973N13.2 | RP11-561B11.6 | DNAJC8P1 |
| RP11-810K23.6 | DUX4L18 | CTD-2062F14.3 | RP11-409I10.2 | AE000661.50 | CTD-2058B24.2 |
| AE000662.93 | RP11-1140I5.1 | PEBP1P1 | RP11-386M24.5 | RP1-261D10.1 | RP11-488C13.6 |
| SEPT7P1 | TRAV30 | RP11-348M3.2 | RP11-270M14.4 | RP4-687K1.2 | LINC00924 |
| CTD-2240H23.2 | RP1-261D10.2 | DUX4L17 | CTD-2017C7.3 | RP11-463C8.4 | RP11-154B12.3 |
| UBE2CP4 | RP11-424I19.2 | RP11-253M7.3 | RP11-133K1.6 | RP11-209K10.2 | CTD-2071N1.1 |
| LINC00928 | CTD-3076O17.2 | SLC35G6 | HNRNPA1P62 | CTD-2555C10.3 | RP11-349G13.1 |
| GOLGA8VP | RP11-361D15.2 | RP11-521C20.1 | RP11-313P18.2 | AC002306.1 | TTTY25P |
| RP11-616M17.1 | RP11-325L12.3 | NDUFA3P4 | AC007950.1 | ANKRD62P1 | CTD-2501E16.2 |
| RP11-122D10.1 | RP11-355N15.1 | CTA-339C12.1 | RP11-37J13.1 | PLCB2-AS1 | RP11-561C5.7 |
| OR11K1P | LINC00596 | HNRNPMP1 | RP11-323I15.5 | TGIF1P1 | RP11-349G13.2 |
| RP11-798K3.2 | RP11-111E14.1 | RP11-90E5.1 | RP11-307C19.1 | RP11-64K12.9 | RP11-815J21.4 |
| CTD-2240J17.1 | RP11-815J21.2 | RP11-505E24.3 | RP11-605F22.1 | RP11-313H3.1 | RP11-430B1.1 |
| RP11-30K9.4 | ISCA1P4 | HERC2P7 | RP11-7M10.2 | RP11-158M2.5 | HNRNPCP3 |
| RP11-593F23.1 | RP11-35O15.1 | CTD-2651B20.4 | NPM1P43 | RP11-403B2.5 | RP11-736N17.8 |
| NIFKP8 | RP11-815J21.1 | CPEB1-AS1 | RP11-540O11.7 | AHCYP7 | NDUFAF4P1 |
| RP11-621H8.2 | RP11-19J5.1 | RP11-244F12.3 | RP11-616K22.2 | RP11-74D7.1 | RP11-661D19.3 |
| UBE2Q2L | CYCSP38 | RP11-685G9.2 | CTD-2651B20.3 | RP11-540O11.4 | RP11-272D12.2 |
| RP11-325E5.4 | CYCSP2 | RP11-335K5.2 | CTD-2329K10.1 | KRT8P23 | RBM17P4 |
| RP11-317G6.1 | RP11-429B14.3 | RP11-540O11.6 | RP11-562A8.5 | RP11-597K23.1 | RP11-467H10.2 |
| RP11-557C18.4 | PCAT29 | RP11-351M8.1 | RP11-89K11.1 | LINGO1-AS1 | RP11-485O10.2 |
| RP11-702L15.4 | RP11-139F4.2 | RP11-499F3.2 | RP11-485O10.3 | RP11-227D13.1 | RP11-752G15.4 |
| NUTF2P6 | CTD-3032H12.1 | CSPG4P11 | RP11-356M20.3 | AC066615.1 | RP11-158M2.4 |
| RP11-90B9.2 | RP11-182J1.13 | RP11-1006G14.2 | RP11-20I23.3 | RP11-359K18.3 | RP11-554A11.9 |
| LYPD8 | RP11-63E9.1 | LINC00567 | HERC2P10 | RP11-467L24.1 | AC097374.2 |
| RP11-177N22.2 | RP11-1012E15.2 | RP11-293B20.2 | RP11-1H8.4 | RP11-343C2.7 | RP6-91H8.2 |
| CTC-527H23.2 | AC022819.3 | RP11-322D14.1 | RP11-218M11.1 | CTD-2323K18.2 | CTD-2651B20.7 |
| RP11-48G14.2 | CTD-2012K14.3 | RP11-170L3.4 | AC009133.15 | CTD-2547G23.2 | RP11-652G5.1 |
| RP11-2E17.2 | CDC37P1 | RP11-252K23.1 | TGFBR3L | RP11-65J21.1 | RP11-1035H13.2 |
| RP11-480I12.10 | CTC-527H23.1 | CTD-2651B20.6 | CTC-527H23.3 | GOLGA2P11 | RP11-182J23.1 |
| CTD-2313J23.1 | RP11-254F7.2 | RP11-615I2.1 | RP11-92G12.3 | RP11-77K12.7 | LINC01070 |
| AOC4P | RP11-883G10.1 | RP11-177B4.2 | LA16C-312E8.2 | RP11-258F22.2 | CSPG4P13 |
| RP11-322D14.2 | RP11-748L13.6 | CH17-53B9.2 | CTC-471J1.2 | MTCYBP28 | RP11-95H11.1 |
| LINC01413 | RP11-2I17.4 | CTD-2022H16.3 | RP11-432I5.6 | RP11-756H20.1 | RP11-108B14.5 |
| CTD-2026K11.2 | RP11-680F20.10 | RP13-395E19.2 | CCDC187 | FRRS1L | CTD-2026K11.3 |
| RP11-588K22.2 | RP11-46D6.4 | EIF4EBP2P2 | C6orf229 | RP11-973H7.1 | RP11-386M24.6 |
| RP11-152P23.2 | DNM1P34 | RP11-343C2.9 | AQP4-AS1 | RP11-540B6.2 | WBP11P1 |
| RP11-1129I3.1 | CTD-2649C14.1 | RP11-800A3.4 | RP11-505K9.3 | RP3-406A7.7 | LA16c-444G7.2 |
| LINC01544 | RP11-96D1.7 | RP11-304L19.2 | NBAT1 | KCNJ18 | RP4-536B24.2 |
| RP11-834C11.11 | CTD-2196E14.9 | RP11-151H2.1 | RP11-297C4.3 | RP11-166B2.7 | CTD-3193O13.1 |
| AC004381.7 | RP11-368N21.1 | CTD-2576F9.1 | RP5-1085F17.4 | CTB-134F13.1 | MT1L |
| RP11-457I16.2 | RP11-63M22.1 | RP11-403N16.3 | TPST2P1 | CTA-363E6.6 | CTD-2055G21.1 |
| DUX4 | RP1-140K8.5 | RP3-522J7.6 | RP11-1166P10.1 | RP11-439L8.5 | RP11-1299A16.3 |
| HERC2P5 | RP1-178F10.1 | RP11-315D16.4 | RP11-368L12.1 | RP11-69H7.2 | RP11-152L20.3 |
| AC004158.3 | RP11-744D14.1 | RP11-278A23.2 | CTD-2576D5.3 | RP11-546O6.4 | RP11-146F11.4 |
| RP11-513N24.1 | RP11-744D14.2 | VN1R67P | RP11-165M1.2 | RP11-329J18.3 | VPS35P1 |
| RP11-107F6.3 | RP11-24M17.3 | RP11-319G9.3 | RP11-256I9.2 | RP11-403P17.5 | RP11-615I2.3 |
| CTA-331F8.1 | RP11-10K17.3 | TAT-AS1 | CKBP1 | RP11-96D1.5 | RP11-554A11.7 |
| RP11-95M5.1 | AC008088.4 | LINC00565 | RP11-196G11.2 | CTD-3154N5.1 | SPCS2P1 |
| RP11-476D10.1 | LINC00557 | RP11-297L17.2 | RP11-333I13.1 | MKRN3-AS1 | LA16c-395F10.2 |
| RP13-122B23.8 | RP11-534L20.5 | RP11-546B15.1 | RP11-1437A8.5 | RP11-116D2.1 | RP11-744K17.1 |
| GS1-279B7.1 | RP11-84D1.2 | CTD-3247F14.2 | AC012322.1 | CTD-2588J6.2 | RP11-209D14.2 |
| RP11-24M17.6 | RP11-673P17.4 | RP11-6O2.4 | RP11-7K24.3 | RP11-554A11.8 | RP11-5N19.3 |
| RP11-435I10.3 | RP11-96K14.1 | TMEM178B | RP11-62H20.1 | ADAMTS7P3 | RP11-58A18.1 |
| RP11-68I18.10 | RP11-356C4.5 | RP11-844G16.1 | CTD-2015G9.2 | RP11-805I24.1 | RNF126P1 |
| RP11-863P13.5 | NIFKP4 | RP11-344A16.2 | CTD-2006K23.1 | RP11-304L19.4 | RP11-517C16.4 |
| RP11-545A16.1 | RP11-44I10.3 | MUC22 | RP11-276H1.2 | RP11-359E8.5 | RP11-653J6.1 |
| RP11-279O17.1 | EIF4BP5 | LINC01616 | RP11-46D6.5 | RP11-626G11.1 | PYCARD-AS1 |
| RP11-388M20.2 | RP11-21B23.1 | RP11-165M1.3 | CTD-2012K14.2 | RP11-578F21.3 | ATP5F1P7 |
| LA16c-390E6.3 | RP11-22C11.2 | RP11-399O19.9 | RP11-347C12.9 | CTD-2576D5.4 | RP11-589C21.5 |
| RP11-626G11.5 | RP11-429B14.4 | RP11-578F21.6 | RP11-63B13.1 | RP11-407P15.1 | RP11-82O18.2 |
| RP11-304L19.8 | CTA-30512.1 | RP11-319G9.5 | RP11-626K17.3 | RP11-16E23.3 | FSCN1P1 |
| RP11-744I24.2 | RP11-553K8.5 | RP1-168P16.2 | HERC2P11 | AC002310.17 | RP11-178L8.3 |
| RP11-426L16.9 | HERC2P8 | CTD-2033A16.1 | RP11-243A14.1 | RP11-79H23.3 | RP11-189E14.4 |
| RP11-357N13.3 | RP11-390D11.1 | RP11-618N24.1 | LA16c-390E6.5 | RP11-489G11.3 | GOLGA6L7P |
| RP11-21L1.1 | BEAN1-AS1 | RP11-1C8.5 | RP11-573G6.6 | RP1-168P16.1 | RP11-719K4.6 |
| RP11-178L8.5 | LINC00165 | CTD-2562G15.2 | CTD-2196E14.3 | RP11-480G7.3 | RP11-307O13.1 |
| RP11-669C19.1 | AC002551.1 | RP11-506E9.3 | RP11-498D10.3 | RP11-328J14.2 | CTD-2354A18.1 |
| DNM1P28 | RP11-350O14.18 | LOXL1-AS1 | RP11-44F14.2 | RP11-691G17.1 | RP11-264L1.3 |
| RP11-303E16.6 | RP11-67A1.2 | RP5-867C24.5 | MTND5P33 | GFY | KYNUP1 |
| RP11-700H6.4 | AC003009.1 | RP11-66H6.3 | RP11-177N22.3 | RP11-81K2.1 | RP11-1260E13.4 |
| DKFZP434A062 | AC139099.5 | CTD-2561B21.10 | CTC-524C5.5 | RP11-455O6.2 | BCAR4 |
| MTCO1P28 | CTD-2033A16.3 | CTC-479C5.11 | CTD-2318B16.2 | RP11-81A22.5 | RP11-462G12.1 |
| RP11-353N14.4 | AC144836.1 | RP11-1260E13.2 | CTD-2561B21.4 | MTND4P34 | CTD-319515.4 |
| LA16c-380H5.2 | CTB-193M12.3 | RP11-676J12.8 | TVP23CP2 | SAMD11P1 | RP11-1099M24.8 |
| RP11-95J11.1 | TXNP4 | LA16c-349E10.1 | CTA-276F8.2 | RP11-334C17.5 | RP11-77K12.5 |
| RP5-1107A17.2 | RP11-156P1.2 | RP13-1032I1.10 | AJ003147.9 | GLIS2-AS1 | RP11-44F14.8 |
| RP11-214O1.1 | U91319.1 | CTD-2545H1.1 | CYCSP40 | CTD-2561B21.11 | RP11-669E14.4 |
| CTD-3060P21.1 | RP5-1029F21.2 | RP11-1260E13.1 | RP11-141J13.3 | RP11-189E14.5 | RPL23AP86 |
| KARSP3 | RP11-457I16.4 | CTD-2047H16.2 | CTD-3088G3.6 | RP11-396B14.2 | RP11-388C12.1 |
| RP11-382B18.3 | AC004878.8 | RP11-95P2.3 | RP11-189E14.3 | RP5-1107A17.4 | RP11-1055B8.2 |
| RP11-542C16.1 | CTC-479C5.6 | LA16c-306E5.3 | RP11-109M19.4 | RP11-204E4.3 | RP11-65J21.4 |
| RP11-530N7.3 | ABHD17AP5 | RP11-822E23.5 | TMEM220-AS1 | RN7SL460P | CTD-2541J13.2 |
| RN7SL471P | RP11-17M24.2 | RP11-973F15.2 | PPIAL4C | RP1-56K13.2 | RP11-338L22.2 |
| RP11-78F17.1 | RN7SL716P | RP11-805L22.3 | AF186192.6 | PPP4R1-AS1 | RP11-269G24.3 |
| RP11-471L13.3 | RP11-321A17.4 | RP11-466A19.6 | OOSP1P2 | RP11-746B8.1 | SNORA4 |
| RP11-321A17.3 | RP11-6N17.9 | RN7SL426P | RP11-334E6.12 | EEF1DP7 | CTD-3010D24.3 |
| RP11-344B2.3 | RP11-53I6.2 | RP11-571L19.7 | RP11-210K20.2 | RP11-180P8.1 | RP11-579O24.3 |
| RN7SL32P | RPll-434D2.11 | RN7SL850P | RP11-504I13.3 | RN7SL42P | RN7SL336P |
| RP11-1124B17.1 | RN7SL744P | RN7SL455P | RP11-20B24.2 | RP11-856M7.4 | RP11-227G15.9 |
| RN7SL784P | SCML2P1 | RP11-20B24.3 | RP11-715F3.1 | RP1-253P7.1 | RP1-37N7.1 |
| RP11-171G2.1 | RP11-283C24.1 | RPL35AP25 | RNU4ATAC | ANXA8L1 | RP11-6N17.1 |
| RP11-474I11.7 | RN7SL488P | CCDC58P1 | RP11-769O8.2 | SNORA77 | CTD-2006O16.2 |
| RP11-338L22.3 | RP4-594I10.3 | RP11-110H1.8 | RP11-861L17.3 | RP11-605F20.1 | RP11-640N20.5 |
| RP4-777O23.2 | WI2-1959D15.1 | RP11-145E5.5 | RP11-464D20.6 | RN7SL793P | RN7SL326P |
| MAPT-AS1 | RN7SL732P | RP11-118G23.2 | CTD-2267D19.4 | RP11-381P6.1 | RP11-498C9.17 |
| RP11-143K11.5 | RN7SL45P | RP11-107K17.2 | RN7SL230P | AC100830.5 | ALOX12P1 |
| RP11-53I6.3 | RP11-661C3.2 | RN7SL262P | RN7SL622P | AC010761.6 | RN7SL664P |
| RN7SL246P | FTLP12 | RP11-70O5.2 | RP11-31I22.2 | RP11-285E9.5 | RP11-31I22.3 |
| CTB-187M2.2 | RP13-638C3.6 | MIR133A1HG | RBP3 | RP11-403A21.3 | RP11-775G23.1 |
| RP11-822E23.7 | PGDP1 | AC104984.4 | RN7SL199P | RP11-466A19.5 | RP11-16C1.1 |
| GLUD1P7 | RP11-1148O4.2 | RP11-361L15.4 | RN7SL656P | RP11-449D8.1 | RP11-131K5.2 |
| FCGR1C | RP11-60A24.3 | AA06 | RP11-183C12.1 | RP11-958F21.3 | RN7SL652P |
| GLUD1P8 | TUFMP1 | RN7SL378P | RP11-5A19.5 | RP11-82O19.2 | RN7SL648P |
| RP11-883A18.3 | RN7SL5P | RP11-354P11.3 | KYNUP2 | RN7SL444P | RN7SL624P |
| RNASEH1P1 | CTD-2349P21.10 | RP11-387H17.6 | RN7SL376P | EEF1E1-BLOC1S5 | RP11-20B24.5 |
| RP11-737O24.2 | MIR5195 | RN7SL577P | RP11-16C1.3 | RP11-567L7.5 | BAHCC1 |
| RN7SL574P | RP11-118E18.2 | RP5-906A24.2 | FBXO36P1 | RP11-354P11.2 | RP11-190I17.2 |
| RP11-855A2.5 | RP11-552O4.2 | RP1-66C13.4 | RP11-100K18.1 | NPM1P2 | RP11-159D12.10 |
| RP11-173M1.4 | RP11-51L5.3 | RP11-344E13.4 | RP11-142O6.1 | RP11-710M11.1 | RP11-180P8.3 |
| RP11-91I8.3 | RP11-822E23.2 | RN7SL220P | RP11-401F2.3 | FAM106CP | RP11-138P22.1 |
| RP11-385D13.3 | RP11-881L2.1 | LINC01443 | RN7SL377P | RP1-71H19.2 | RP11-583F2.5 |
| RP11-856M7.6 | RP11-927P21.2 | RP11-1376P16.1 | RP11-160E2.21 | RP11-30L3.2 | RP11-214O1.2 |
| RP11-855A2.3 | RP11-1109M24.7 | RP11-678G15.2 | RP11-218M11.6 | RP1-77H15.1 | RP11-6N17.2 |
| RP11-599B13.7 | ITM2BP1 | RP11-744K17.8 | RP11-567M16.5 | CTB-171A8.1 | RP5-1022P6.7 |
| SLC25A6P4 | ZNF793-AS1 | CTB-70G10.1 | RP11-400F19.8 | AC104532.3 | RP11-820I16.3 |
| AC006126.3 | RP11-712P20.2 | RP11-116O18.1 | CTC-232P5.1 | RP11-354P11.8 | LLNLR-284B4.2 |
| RP11-127I20.7 | RP11-703I16.3 | RP11-886H22.1 | AC002984.2 | RP11-800A18.4 | TCF4-AS1 |
| RP11-807E13.2 | RP11-384O8.1 | RP11-58G13.1 | CTD-3193K9.4 | RP11-128P17.2 | CTB-30L5.1 |
| AC005336.4 | AC005943.2 | RP11-127I20.5 | RP11-666A8.9 | KRT18P61 | RP11-15J10.9 |
| AC005789.9 | PCAT19 | CTD-2587H24.4 | CTB-129P6.11 | CTD-3064H18.2 | RP11-64C12.7 |
| CTB-31O20.6 | RP11-449J21.5 | AC005954.3 | AC005262.3 | RP11-383D22.1 | SDHDP1 |
| RP11-78A19.3 | RP11-527L4.9 | RP11-397A16.3 | PHF5CP | RP11-813F20.4 | RP11-693L9.2 |
| CTD-2132N18.2 | RP11-49K24.6 | AC005786.5 | HNRNPA3P16 | CTB-184G21.3 | RP11-64C12.6 |
| RP11-209M4.1 | ZNF790-AS1 | RP11-1151B14.4 | CTD-2008P7.9 | CTC-543D15.3 | CTB-129P6.4 |
| RP5-951N9.2 | RP11-567M16.1 | CTB-186G2.1 | RP11-703I16.2 | RP11-178C3.1 | LINC01415 |
| AC002398.12 | RP11-411B10.3 | RP11-1094M14.12 | RP11-1094M14.8 | CTD-2231E14.5 | CTC-548K16.5 |
| RP11-1151B14.3 | TCF4-AS2 | CTC-296K1.4 | CTD-2319I12.2 | AC005616.2 | AC006116.15 |
| DNAH17-AS1 | CTC-454I21.4 | RP1-37N7.5 | SMUG1P1 | CTB-39G8.2 | RP11-13K12.5 |
| LOC440461 | RP11-126O1.4 | CTC-398G3.6 | RP11-815J4.5 | CTC-518P12.6 | CTD-3116E22.6 |
| RP11-845C23.2 | RP11-13K12.1 | ZNF285 | CTD-2621I17.6 | RP11-815J4.7 | LINC00868 |
| AC007229.3 | AC005779.2 | AC005264.2 | RP5-1052I5.2 | CTC-503J8.4 | STK25P1 |
| AC104532.4 | CTD-2540B15.11 | RP11-322E11.5 | RP11-687F6.4 | NDUFA3P1 | RPIAP1 |
| RP11-323N12.5 | CTD-3116E22.7 | AC138472.6 | RP11-219G17.6 | RP11-1136J12.1 | AC138430.4 |
| RP11-64C12.4 | CTD-2008P7.10 | RP11-686D22.3 | RP11-95O2.1 | RP11-973H7.4 | RP11-807E13.3 |
| RP11-358B23.1 | CTB-55O6.4 | AC010525.5 | RP11-1030E3.1 | ERVK-28 | CTD-2013N17.4 |
| RP11-108P20.3 | CTD-2105E13.6 | RP11-95O2.5 | RP11-147L13.7 | AC024592.9 | CTD-2189E23.1 |
| AC010761.14 | RP11-64C12.3 | RP4-604K5.3 | HMGB2P1 | AC061992.2 | RP11-686D22.8 |
| RP11-358B23.5 | RP11-559N14.6 | RP11-677O4.4 | RP11-2N1.2 | RP11-154H12.3 | SLC25A36P1 |
| CTB-55O6.10 | CTD-2659N19.2 | RP11-49K24.5 | CTB-191K22.5 | CTB-133G6.2 | RP5-1023B21.1 |
| RP11-120K24.3 | ZSCAN5D | CCDC177 | CTD-2525I3.2 | CTD-2620I22.3 | CTB-189B5.3 |
| CTD-252513.3 | CTC-244M17.1 | AC067969.2 | CTD-3032J10.2 | BNIP3P40 | TOP1P1 |
| SLC6A14 | CTB-60B18.12 | VN1R84P | CTD-3193O13.11 | AC003002.4 | Metazoa_SRP |
| AC010524.4 | AC004076.9 | RP11-556I13.2 | AC002985.3 | FKBP4P6 | CTB-60B18.18 |
| CTD-2623H2.7 | CTD-3222D19.11 | BNIP3P25 | LRRC2-AS1 | L34079.2 | CTD-2568A17.8 |
| CTC-360J11.4 | FENDRR | TRMT112P6 | CTD-2626G11.2 | AC011551.3 | AC011530.4 |
| BNIP3P27 | BNIP3P38 | ABC7-42418200C9.1 | AC018755.18 | CTD-2545M3.8 | RP11-32B5.8 |
| AC004076.7 | AC005523.3 | AC003956.1 | RP11-514A9.1 | VN1R80P | CTD-2207O23.10 |
| CTB-52I2.3 | RP1-122P22.4 | RP11-739B23.1 | CTB-33G10.11 | CTD-3187F8.7 | CTB-60B18.10 |
| LETM1P2 | FAM231B | CTD-2553L13.4 | RP11-588H23.5 | CTC-444N24.9 | CTD-2105E13.14 |
| CTD-2561J22.1 | CTB-61M7.2 | CTD-2396E7.7 | CTD-3030D20.1 | AC104534.2 | ADGRE4P |
| CTC-450M9.1 | VN1R90P | CITF22-62D4.1 | SIGLEC22P | CTB-167G5.6 | PNMA6B |
| CTD-3099C6.7 | CTD-3233P19.8 | CSAG3 | RP11-886P16.6 | AC005387.3 | CT45A1 |
| ERVV-2 | AC005253.4 | SPANXN2 | RP11-439A17.5 | RP1-172N19.1 | KRT18P40 |
| MTRNR2L12 | CTC-513N18.6 | CTC-360G5.6 | CTD-2528A14.5 | RP11-256I23.2 | CTD-3222D19.10 |
| AC010646.3 | CTD-2525I3.5 | TRABD2B | BRI3BPP1 | RP3-431P23.5 | CTC-218B8.3 |
| AC005387.2 | ZNF350-AS1 | CTB-83J4.2 | CTD-2105E13.15 | CTD-2527I21.7 | AC010522.1 |
| CTD-2278110.6 | CTC-453G23.4 | CTC-312O10.2 | CTD-2616J11.11 | LINC01224 | BNIP3P8 |
| CTD-3032J10.3 | Z69720.2 | CTB-174O21.2 | DMRTC1 | CYP4F23P | CLIC4P2 |
| CTD-2192J16.21 | CTD-3222D19.5 | TDGF1P7 | CTB-167G5.3 | RP11-542M13.2 | CTD-2521M24.11 |
| AC005614.5 | CTB-60E11.9 | CTB-180A7.3 | GLTSCR1-AS1 | SCGB2B3P | RP11-11A5.1 |
| CTD-2537I9.16 | CTD-2587H19.3 | AC004754.3 | RP11-463C8.7 | RP11-266J6.2 | RP11-477H21.2 |
| RP5-1139B12.2 | RP4-740C4.5 | RP11-178L8.9 | RP6-99M1.3 | RP11-442H21.2 | RP6-91H8.3 |
| RP5-1139B12.3 | RP11-394O4.5 | RP11-214K3.20 | RP11-2B6.3 | RP11-248J23.6 | RP11-324I22.3 |
| RP11-148O21.6 | SPACA6P-AS | RP11-498E2.9 | RP3-426I6.5 | RP3-430N8.10 | RP11-130L8.2 |
| RP11-282O18.7 | RP3-430N8.11 | AF003625.3 | RP11-272L13.3 | RP3-449M8.9 | RP11-218C14.8 |
| RP11-93H12.4 | CTD-2132N18.4 | RP11-380L11.4 | RP11-685G9.4 | RP11-416A14.1 | RP11-214K3.22 |
| WI2-80269A6.1 | MIR222HG | RP11-750H9.7 | RP11-351121.11 | KB-1958F4.1 | AP000473.8 |
| RP11-214K3.18 | RNU11 | RP11-245P10.6 | RP11-326C3.14 | RP5-1139B12.4 | VTRNA2-1 |
| RP11-118F19.1 | RP11-214K3.23 | TERC | RP11-568J23.6 | LA16C-380H5.3 | RP11-494H4.3 |
| RP1-170O19.24 | RP11-803D5.4 | RP11-89C3.2 | AC015849.2 | RP11-779O18.4 | CTC-471J1.10 |
| RP11-420B22.2 | RP11-17P16.2 | RP13-122B23.9 | RP11-731D1.4 | RP11-453E17.4 | RP11-566F5.1 |
| STX8P1 | RP11-461L13.4 | GS1-165B14.2 | B0RCS7-ASMT | RP11-697E2.10 | BNIP3P9 |
| UNGP3 | RP11-547M24.1 | RP11-470B22.1 | CTC-412M14.5 | RP11-44D5.1 | RP11-351O1.4 |
| RP11-1348G14.6 | OR7E108P | RP11-511111.2 | RP11-972P1.8 | RP11-411B10.8 | IGHV3-29 |
| RP11-230C9.3 | MKI67P1 | YTHDF2P1 | RP11-24C14.1 | RP11-145P16.3 | RP11-311H10.7 |
| RP11-15E18.5 | RP11-225N10.3 | RP11-362B23.1 | RP11-713H12.2 | RP1-134O19.3 | LOC401913 |
| RP11-373D23.2 | TSIX | HNRNPA1P67 | CTB-17911.3 | CTC-512J12.7 | RP11-349G13.3 |
| RP11-381K20.4 | RP11-568J23.7 | RP11-568G11.5 | RP11-231P20.5 | RP4-802A10.1 | IGKV2OR2-1 |
| DEAR | RP11-138E2.1 | CTD-2324A24.2 | AD001527.7 | PDHA1P1 | RP13-685P2.7 |
| RP11-501C14.9 | PDLIM1P1 | RP11-632K20.8 | DPRXP6 | RP11-416H1.1 | RP11-574K11.27 |
| CHCHD2P11 | RP11-730B22.1 | RP3-466P17.2 | RP1-168P16.3 | CTC-571O20.1 | RP11-674I16.1 |
| VDAC1P5 | RP11-420K10.1 | RP11-609L3.1 | RP11-311B14.1 | GPR75-ASB3 | RP11-272L13.4 |
| RP11-27M5.1 | SRD5A3P1 | XXbac- | RP5-879K22.1 | RP5-966M1.5 | HSPE1P7 |
| | | BPGBPG24O18.1 | | | |
| RP11-62H7.3 | RP11-54C4.2 | RP11-137J7.3 | RP11-123C21.2 | RP11-439C15.5 | RP11-844P9.4 |
| CTHRC1P1 | CTC-539A10.7 | RP11-60E8.2 | RP11-963H1.1 | RP5-933K21.3 | RP11-1113N2.4 |
| CTD-2342N23.2 | CTD-2553L13.7 | RP11-479I16.2 | RP11-457M11.6 | PHBP20 | NAMA |
| SUMO2P14 | RP11-223J6.1 | RP11-123C5.5 | RP11-33A14.3 | RP5-966M1.4 | YWHAQP7 |
| RP11-479F13.1 | RP11-642C5.1 | RP11-409O11.3 | RP11-629P16.1 | RP5-855F16.1 | RP4-555D20.3 |
| RP13-216E22.5 | RP3-523E19.2 | RP11-369K17.2 | KRT18P9 | RP5-894D12.4 | SUMO2P15 |
| RP11-424I3.1 | CTD-2117L12.3 | RP11-1109F11.5 | TCEB1P33 | CTC-412M14.6 | DPRXP2 |
| RP11-1152H14.1 | RP11-1109F11.3 | RP5-891H21.5 | RP11-797J4.1 | RP11-701H24.7 | RP11-843B15.3 |
| RP11-81A1.8 | RP3-483K16.4 | RP11-253I19.4 | RP11-33I11.3 | C8orf59P2 | RP11-382D12.2 |
| RP1-161P9.5 | IGKV2OR2-2 | MZT1P2 | PRR13P3 | RP11-751K21.1 | RPL23AP90 |
| RP11-295B17.6 | MMP28 | CT45A2 | CICP24 | RP4-799G3.1 | CTC-559E9.9 |
| RP11-603B24.6 | RP11-36B6.1 | ARF4P1 | RP11-692M12.5 | RP11-579D7.8 | RP4-665N4.8 |
| RP11-52M17.1 | RP3-418A9.3 | HSPE1P14 | ACTG1P22 | RP11-435I10.5 | RP11-289A15.1 |
| MARK3P1 | RP11-270B14.1 | RP11-95I16.4 | RP11-1E1.2 | RP11-564A8.8 | RP11-1259L22.2 |
| RP11-393B14.2 | BNIP3P20 | RP11-312P12.3 | CTD-2256P15.5 | RP11-169K17.4 | CTD-3020H12.4 |
| RP11-787I22.3 | RP5-1182A14.5 | CTB-113I20.2 | RP4-680D5.9 | XXbac- | RP11-87N3.6 |
| | | | | BPGBPG55C20.3 | |
| RP11-254F7.3 | SNORA51 | RP11-426L16.10 | RN7SKP4 | RP5-894D12.5 | RP11-337N6.2 |
| RP11-1C8.7 | RP11-436D23.1 | CTD-2228A4.1 | RP4-789D17.5 | RP11-44N12.5 | RP11-63L7.5 |
| RP11-222K16.1 | CTD-3064M3.7 | CTD-2140B24.6 | RP11-134K13.4 | U47924.29 | AC226119.4 |
| RP11-28H5.2 | RP11-345P4.10 | CTC-350I8.1 | CTB-40H15.4 | FAM231D | RP11-332J15.4 |
| CTD-2376I4.2 | RP11-542A14.2 | CTD-2653M23.3 | XXcos-LUCA11.5 | AC005754.8 | CTD-2366F13.2 |
| KB-1836B5.4 | RP11-360I2.1 | RP11-107C16.2 | LINC01607 | RP11-465B22.8 | RP11-350N15.6 |
| RNU4-5P | PROX1-AS1 | CASC15 | RP11-387M24.5 | RP4-657D16.6 | RN7SL803P |
| AC004775.5 | RP3-500L14.2 | RP11-11N5.3 | RP5-855D21.1 | KB-1958F4.2 | RP11-346C20.4 |
| RP11-489E7.4 | RP11-305P22.9 | TPTE2P2 | RP11-394A14.4 | RP11-506K6.4 | KB-1517D11.4 |
| RP1-151F17.2 | RP11-307L14.2 | RNU4-89P | RP11-359I18.5 | RP11-446N19.1 | RP11-25O10.2 |
| RP11-441F2.5 | RP11-556O9.4 | RP11-284F21.10 | RP11-185E12.2 | RP3-511B24.5 | RN7SL778P |
| RP11-54O7.16 | RP1-225E12.3 | RP1-136B1.1 | RP11-95G17.2 | RP11-158G18.1 | RP1-317E23.7 |
| RP11-474O21.5 | RP11-169K17.3 | RP11-284J1.1 | RP5-1024N4.4 | RP4-535B20.4 | RP11-96C23.14 |
| RP11-255P5.2 | RP11-351J23.2 | RP11-324L17.1 | RP11-480C16.1 | RP11-462G22.2 | OR10AH1P |
| RP11-737O24.5 | GS1-259H13.13 | AP000320.7 | RP11-190A12.8 | PCDHB16 | RP11-797D24.4 |
| RP11-216L13.18 | AC004471.10 | RP11-290M5.4 | RP1-63G5.8 | RP11-78A19.4 | RP11-534C12.1 |
| CTB-161C1.1 | OR7E47P | KB-208E9.1 | RP11-222G7.2 | RP11-155D18.12 | RP11-74E22.5 |
| RP11-433A10.3 | RP1-170O19.23 | U91328.22 | RP11-15I20.1 | RP6-74O6.6 | RP11-91K8.5 |
| OR10AE3P | RP1-137D17.2 | RP11-332H14.1 | RP5-1103G7.10 | RP11-98C1.2 | RP1-309K20.6 |
| RP11-299H21.1 | RP11-121A8.1 | RP11-724N1.1 | RP11-330O11.3 | RP11-500M8.7 | RP11-307C18.1 |
| OR7A19P | RP11-190C22.9 | RP11-329B9.4 | RP11-284F21.11 | RP5-1180E21.4 | RP11-85E16.1 |
| RP11-150D20.5 | ARL2-SNX15 | CTD-2303H24.2 | RP11-508N22.13 | RP11-285F16.1 | FAM95C |
| ATF4P2 | RP4-569D19.8 | RP11-834C11.14 | RP11-834C11.12 | CTA-714B7.7 | GRIN2B |
| RP11-1275H24.3 | RP11-201K10.3 | RP11-532L16.3 | RP3-508I15.20 | RP11-350G8.9 | RP11-350J20.12 |
| RP11-290D2.6 | RP11-406H21.2 | RP11-104L21.3 | RP11-307N16.6 | CTB-96E2.2 | RP3-510H16.3 |
| RP11-20I20.4 | CTD-3148I10.15 | AP000692.10 | RP4-549L20.3 | RP11-107N15.1 | CTC-432M15.3 |
| RP11-644N4.1 | RP5-1180E21.5 | KB-7G2.8 | SERPINA3 | ZBTB8B | RP6-109B7.5 |
| RP11-445N20.3 | CTB-13L3.1 | RBAKDN | RP11-141M3.6 | RP5-899E9.1 | RP11-466F5.10 |
| RP11-96C23.15 | RP4-597N16.4 | RP1-170O19.22 | AC006946.17 | RP11-54O7.18 | RP5-1147A1.2 |
| RP11-305O4.3 | RP11-102N12.3 | RP11-108L7.14 | RP4-671G15.2 | RP4-621B10.8 | RP11-80H18.4 |
| RP11-50D16.4 | HIST1H4K | OR4F16 | CTD-2049O4.2 | CTD-2024I7.18 | RP11-426C22.7 |
| RP11-861L17.4 | RP11-433A19.2 | ST7-OT4_1 | RP11-392A14.9 | CTD-2564P9.3 | RP11-66N24.7 |
| uc_338 | MIR142 | RP11-15E1.4 | RP11-406H23.5 | RP11-566K19.8 | RP5-837J1.6 |
| CT45A7 | LHX1 | RP5-1021I20.8 | U1 | LLfos-48D6.2 | RP11-420C9.1 |
| RP11-370I10.11 | CEACAM20 | RP11-620J15.4 | RP11-81H14.1 | RP11-394O2.3 | AL078471.5 |
| RP11-133K1.12 | RP11-174N3.4 | RP11-603J24.21 | RP11-227D13.5 | RN7SL733P | RP11-346J10.3 |
| CTD-2306A12.1 | Metazoa_SRP | RP11-332H18.7 | RP11-585P4.6 | Metazoa_SRP | RP11-732M18.4 |
| RP11-340F14.6 | RP11-570L14.1 | ABC12-47964100C23.1 | RN7SL539P | RP11-140H17.2 | FAM27AP1 |
| RP11-474C8.8 | Metazoa_SRP | AC018737.1 | RP11-407N8.6 | AL133243.3 | RP4-568C11.4 |
| LLNLR-284B4.1 | KB-68A7.1 | RP11-357P18.2 | RP4-545L17.12 | ADRA2B | RP11-20G13.5 |
| SNORA71E | PCA3_1 | RP11-239C9.1 | RP11-717F1.2 | RP11-227G15.10 | TTC28-AS1_3 |
| RP11-40B20.1 | RP11-203I16.7 | RP11-216L13.21 | LL22NC03-84E4.13 | RP5-994D16.11 | RP11-154H23.5 |
| CTD-2540L5.9 | RN7SL196P | RP11-972P1.10 | C8orf89 | RP11-323P17.2 | RP11-58A18.2 |
| RP4-583P15.16 | Metazoa_SRP | RP5-965G21.5 | CTC-338M12.13 | RP11-146B14.1 | SEBOX |
| RP1-62D2.4 | RP11-434C1.6 | CTD-3035K23.7 | RP11-819M15.2 | RP11-474P2.5 | ST7-OT4_4 |
| CTC-260E6.12 | RP11-49G2.3 | TBC1D3J | RP11-986G18.2 | Metazoa_SRP | RP11-2C24.7 |
| Metazoa_SRP | RP1-47A17.1 | RP11-618L22.1 | CCL23 | TCEB3CL2 | RP1-59D14.10 |
| AC131056.3 | RP11-407N8.5 | Metazoa_SRP | Metazoa_SRP | RP11-49I11.4 | RP11-136L23.2 |
| RP11-214K3.25 | RP11-65I12.1 | CTB-167G5.7 | CTD-2311M21.4 | RP5-1009E24.9 | SPDYE13P |
| RP3-492J12.2 | RN7SL600P | CTB-58E17.2 | MIR6753 | AC233755.1 | CH17-262O2.1 |
| GNAS-AS1_5 | SNORD91B | RP11-442O1.3 | RP11-711K1.8 | RP11-182J1.17 | CH17-219N22.1 |
| RP11-324D17.3 | CCL16 | RP11-385J1.3 | RP11-4B16.3 | AC008984.2 | RN7SL544P |
| FTX_3 | RP11-428O18.6 | Metazoa_SRP | RP11-234G16.4 | SPDYE19P | RP11-83N9.6 |
| CTD-2017F17.1 | RP3-425C14.6 | RP11-329N22.2 | ZEB2_AS1_1 | RP11-346J10.2 | RP11-125A15.3 |
| HNF1B | RP11-844G16.3 | RP11-126O1.6 | LA16c-360A4.1 | RP11-569G13.3 | RP11-793H13.12 |
| RP11-322E11.2 | RNVU1-19 | TCEB3CL | RP11-3K24.3 | Metazoa_SRP | XKR5 |
| RP11-802O23.4 | RP11-35J10.6 | RP11-12A20.12 | RP11-209D20.2 | KCNQ1OT1_1 | RP11-876N24.7 |
| RP11-1021O19.2 | CCL15-CCL14 | U47924.32 | RP4-777L9.3 | RP11-100G15.12 | HNRNPCL2 |
| RN7SL736P | HTR1DP1 | RP4-697K14.15 | RP11-479O9.3 | CTC-421K24.1 | LINC00162 |
| IKBKGP1 | RP11-497G19.7 | Metazoa_SRP | RP1-278C19.8 | uc_338 | EIF3FP1 |
| AC108479.3 | RP11-180M15.6 | CTD-2240J17.4 | REXO1L12P | KCNQ1OT1_5 | CTD-2027I19.3 |
| RN7SL197P | CH507-152C13.3 | RP11-507M3.1 | RP11-734B5.2 | RP11-455O6.8 | RP4-790G17.7 |
| CTD-2008A1.3 | RP11-154D6.2 | SNORA43 | TP73-AS1 | LINC00226 | RP4-781B1.5 |
| PIK3R6 | SCARNA10 | OR4E1 | CTB-91J4.1 | CTB-32O4.3 | AC011043.1 |
| RBBP4P3 | GTF2IP8 | UPF3BP1 | RP11-241M13.2 | RP5-928E24.4 | RP11-106D4.3 |
| Metazoa_SRP | FLJ36000 | VN1R9P | RP11-490B18.5 | CCL14 | HIST1H2BB |
| RP11-157L3.9 | RP11-266K4.13 | NPPA-AS1_1 | RP11-210K20.6 | RP11-400G3.5 | Metazoa_SRP |
| BMS1P14 | CMB9-94B1.2 | RP11-295H24.5 | RAB7B | RP11-300D11.3 | DACH1 |
| Metazoa_SRP | RP11-35J10.7 | IGKV1OR1-1 | RP5-1057I20.5 | RP13-977J11.9 | RP11-3D4.4 |
| Metazoa_SRP | YWHAEP7 | Metazoa_SRP | RP11-381O6.1 | RP11-500G22.4 | RP11-461A8.5 |
| CH17-125A10.1 | RP5-1116H23.5 | CTD-2515H24.2 | RP11-290H9.5 | RP11-374M1.10 | RP11-19G24.2 |
| Metazoa_SRP | CH17-360D5.2 | RP5-875H18.9 | AL662801.1 | RP4-608O15.3 | GSTTP1 |
| RP11-318K12.1 | HYDIN2 | CTD-3128G10.7 | LA16c-359F1.1 | MIAT_exon5_3 | RP11-476H16.1 |
| RP3-453C12.15 | SNORA71 | RP11-101P17.15 | RP5-1116H23.6 | RP11-520B13.8 | RP11-113A11.3 |
| CTD-2024F21.1 | SNORA9 | RP13-554M15.8 | RP11-89G4.1 | AC007325.2 | CH17-125A10.2 |
| RP11-74E22.8 | SDR42E1P2 | RP4-550H1.7 | RP11-153I24.4 | Metazoa_SRP | RP13-580B18.2 |
| RN7SL556P | ATP5A1P10 | AP003041.1 | RP11-699C17.1 | RP11-157L3.3 | RP11-462D18.4 |
| RP11-141M1.4 | Metazoa_SRP | CH17-385C13.2 | REXO1L6P | RP5-1116H23.3 | XXbac- |
| | | | | | BPG154L12.5 |
| RP11-256K9.1 | RP11-538C21.2 | CTB-96E2.10 | RN7SL473P | RN7SL286P | AC213203.1 |
| AL022345.10 | PKD1L3 | Metazoa_SRP | RP13-347D8.7 | RP11-298C2.1 | RP11-746P2.6 |
| RP11-73M11.3 | RP5-1057B20.3 | UPF3BP4 | RP3-468O1.6 | CTD-2525I3.8 | CTC-268N12.3 |
| CTD-3116E22.8 | RP11-12A20.8 | RP11-295M3.4 | RP11-255M2.3 | AC009133.22 | Metazoa_SRP |
| XXbac- | RP11-459O16.9 | Metazoa_SRP | ZFAT-AS1_2 | FP325317.1 | RP11-693J15.6 |
| B33L19.15 | | | | | |
| Metazoa_SRP | GOLGA6L17P | RP11-757O6.7 | H2AFB3 | ABC7-42404400C24.1 | CTD-2588E21.1 |
| RN7SL763P | Metazoa_SRP | AC005752.10 | SMAD5-AS1_1 | GOLGA6L22 | FAM230A |
| RP11-33N14.3 | RP11-467P22.5 | RP11-122G18.9 | SNORA76C | RP5-851M4.1 | RP11-333J10.2 |
| RN7SL478P | uc_338 | Metazoa_SRP | RP11-90D4.4 | HOXA11-AS1_4 | XXbac- |
| | | | | | B562F10.12 |
| RP11-428P16.3 | RP11-735A19.3 | SNORA67 | RP11-356M6.1 | RP11-426C22.8 | RP11-157L3.11 |
| RP11-35O15.2 | SOX2OT_exon4 | CTD-2240J17.2 | NEAT1_2 | TEX28 | RP11-466M21.1 |
| RP11-15E1.6 | RP11-525G12.1 | U1 | RP11-972P1.11 | AC116050.1 | NEAT1_1 |
| CTD-3028N15.3 | RP11-476I15.5 | CH17-262O2.2 | SSTR3 | RP13-766D20.4 | CNTNAP3P5 |
| RP5-906C1.1 | uc_338 | AC020956.3 | UPF3BP3 | Metazoa_SRP | SNORA61 |
| RP11-55J15.2 | RP3-324O17.8 | CT45A6 | GRAMD4P3 | RP11-1006G14.5 | XXbac- |
| | | | | | BPGBPG34I8.2 |
| CTD-2267D19.7 | OR7E104P | MIAT_exon1 | RP11-255P5.3 | CTD-2240J17.3 | RP11-261C10.8 |
| RP11-66B24.9 | RP11-165D6.1 | RP11-19E18.2 | RP11-144H23.2 | UPF3BP2 | RP11-123C21.3 |
| RP11-108K14.12 | RP11-432I13.6 | IGKV2OR2-8 | OR4F29 | CTA-313A17.5 | RN7SL113P |
| TBC1D3E | RP11-405M12.3 | RP11-506H21.5 | CTD-2318O12.1 | CTC-268N12.2 | CDK2AP2P1 |
| RP11-434C1.4 | RP11-49K24.9 | IQCA1L | LA16c-352F7.1 | AC000403.4 | Metazoa_SRP |
| Metazoa_SRP | RP5-1116H23.4 | AC007325.4 | Metazoa_SRP | NPPA-AS1_2 | RP11-567L7.3 |
| PRO1804 | RP11-108A14.1 | RP11-183A22.3 | CTC-448F2.7 | CTD-2342N23.1 | RP11-65F13.3 |
| RP11-523J2.1 | RP11-401P9.7 | CTD-2299N12.1 | RP11-137O10.1 | RP11-139H14.5 | AC104981.1 |
| SMG6-IT1 | RP11-67H24.3 | RP11-39H3.2 | RP5-849H19.3 | RP11-662I13.3 | CTD-3022L24.1 |
| RP11-426J5.3 | RP11-762L8.6 | RP11-1430O6.1 | RP11-360A10.1 | RP11-697K23.3 | RP11-391L3.4 |
| RP11-131P10.2 | RP11-81K2.2 | AC007787.3 | CTC-270D5.1 | CTGLF11P | CH507-216K13.1 |
| CTD-253719.15 | CH17-140K24.5 | RP11-83B20.3 | RP11-322E11.3 | RP1-167O22.1 | RP11-461F11.2 |
| AL356289.1 | RP11-823P9.4 | CTA-243E7.1 | RP11-83B20.5 | RP11-394B2.3 | RP11-76C10.4 |
| RP11-264L1.2 | LINC01451 | AC139099.7 | RP1-163M9.7 | RP11-43D4.3 | RP11-287D1.5 |
| CTA-113A6.1 | RP11-632P5.1 | RP5-991G20.6 | CTA-212A2.3 | RP11-263G22.1 | GPR1-AS |
| RP11-543H23.1 | CH507-152C13.5 | AC019206.1 | RP11-195O1.5 | CMB9-22P13.2 | RP11-536C12.1 |
| LA16c-380A1.2 | CTD-3137H5.5 | RP11-573M3.5 | CTA-345G4.1 | RP11-64C12.5 | CTD-3149D2.2 |
| CH507-145C22.4 | RP11-75I2.3 | RP11-83B20.6 | RP11-864N7.4 | RP11-776B3.1 | AGPAT4-IT1 |
| RP11-475D10.4 | RP11-360N9.3 | RP1-247F3.1 | RP11-1182P23.5 | RP11-650L12.4 | CH17-140K24.6 |
| AC003973.3 | AC069063.2 | AF127577.13 | RP11-81A1.4 | RP11-369J21.12 | CTD-2353F22.2 |
| RP11-849I19.2 | RP11-382B18.1 | LINC00244 | RP11-641J8.1 | RP11-258F1.2 | RP11-190A12.9 |
| RP11-218M11.7 | CTD-2576N18.1 | LA16c-313D11.13 | AC005915.1 | AC006026.2 | AC005480.1 |
| RP11-44D5.2 | RP11-386I8.5 | AC009065.4 | CTD-2286N8.1 | RP11-391L3.3 | RP11-91I8.2 |
| RP13-977J11.3 | RP11-391A7.1 | RP11-392A14.8 | CTD-2576D5.1 | RP11-72I2.2 | RP11-354E23.4 |
| AC243945.1 | RP11-676B18.1 | RP11-575M22.1 | GS1-306C12.1 | RP11-83B20.2 | CTB-96E2.6 |
| AC009022.1 | RP11-133K1.8 | OR4X2 | AC087762.1 | AC005786.6 | AC002044.1 |
| RP4-566L20.1 | RP11-637C24.5 | RP11-502F1.1 | RP11-348J12.5 | RP11-235C23.6 | AC005796.2 |
| RP11-399C16.3 | RP11-573G6.4 | RP11-989F5.4 | RP11-521H3.3 | RP11-507J18.5 | RP11-1086I4.2 |
| RP11-278J6.5 | RP11-274A11.5 | RP11-755E23.2 | MAPT-IT1 | ECSCR | CH507-9B2.8 |
| RP11-485M7.2 | RP11-71L14.3 | CTD-2028E8.2 | CTD-2335A18.2 | CTD-3035K23.3 | CTA-212A2.2 |
| LLNLF-158E9.1 | RP11-81A1.7 | RP11-416H1.2 | AC124312.1 | RP11-79C6.2 | RP11-44F14.9 |
| CH507-42P11.3 | CTD-2169H9.1 | RP11-278H7.5 | AC004813.1 | RP11-586K12.2 | RP11-499E14.1 |
| RP11-665E10.2 | RP11-586K12.1 | RP11-317F20.3 | CTC-510F12.3 | AC005538.3 | RP3-495K2.4 |
| CR392000.1 | RP11-394B2.7 | AC010287.1 | RP11-142L16.2 | RP11-775C24.4 | RP11-648O15.2 |
| RP11-504G3.2 | RP1-228P16.9 | RP11-20F18.1 | CH507-216K13.2 | AC005618.9 | RP11-539L10.4 |
| RP11-570J4.1 | RP11-420N3.3 | CTB-151G24.2 | CTD-2315M5.2 | RP11-483E17.1 | RP11-264L1.1 |
| FLJ42393 | GHc-857G6.7 | BIRC6-AS2 | RP11-360N9.2 | RP11-481J8.3 | FO538757.3 |
| RP11-145E17.3 | LA16c-312E8.4 | RP11-243M5.5 | CH17-76K2.6 | RP11-483I24.1 | WI2-85898F10.2 |
| RP11-141B14.2 | RP5-1181K21.4 | RP11-472K17.3 | CTD-2553L13.5 | RP11-243M5.4 | GABARAPL3 |
| CH17-140K24.8 | CTC-436P18.4 | RP11-76C10.3 | CTB-139P11.2 | AL353997.3 | AC005086.2 |
| CTD-2123J17.2 | RP4-671O14.7 | RP11-118E18.1 | CH17-140K24.2 | RP11-10J21.2 | RP11-540O11.8 |
| DNM1P41 | AL133335.1 | CTC-463A16.1 | RP11-61K12.2 | RP11-5G9.6 | LA16c-380F5.1 |
| RP11-907D1.3 | RP11-361H10.5 | CTC-312O10.3 | AC002044.3 | OR1B1 | CH507-42P11.7 |
| RP11-334E15.1 | RP11-423G4.8 | RP11-133M8.3 | RP11-158L12.6 | AC093642.1 | CTD-3137H5.4 |
| AC073333.1 | AC016142.1 | RP11-331F4.5 | RP11-314N13.9 | RP11-354E23.3 | CH507-210P18.3 |
| RP5-906A24.1 | CH507-42P11.6 | RP11-319G9.1 | CH507-42P11.5 | RP11-243M5.2 | RP11-504I13.2 |
| RP11-174G17.3 | RP11-454F8.3 | RP11-67L14.1 | AL590235.1 | RP11-321N4.4 | RP11-159D12.3 |
| RP11-145P16.2 | RP11-574K11.26 | AL049794.1 | RP5-1037N22.2 | MTCO1P1 | RP11-44N21.3 |
| RP11-466A19.4 | RP11-6L6.6 | RP5-1007F24.1 | RP11-340F16.2 | RP11-560F18.1 | RP11-556H2.1 |
| RP11-314C9.1 | RP11-340L3.1 | FRG1KP | RP11-177H2.1 | RP11-78J21.6 | RP11-583F2.7 |
| RP11-64C12.1 | CTD-3076M17.1 | RP11-665E10.4 | RP1-111J24.1 | RP11-1110F20.1 | RP11-545D22.1 |
| RP11-667K14.14 | RP11-15F12.6 | RP11-438L7.1 | CH507-152C13.4 | RP5-1065O2.4 | RP11-83B20.4 |
| AC092159.1 | RP11-9H20.2 | RP13-204A15.5 | RP11-93O14.3 | CTC-503J8.8 | RP5-898I4.1 |
| RP11-42F12.1 | RP11-818O24.2 | AC007326.10 | CITF22-92A6.2 | RP11-446E24.3 | AC104057.1 |
| Metazoa_SRP | SNORA16A | AC092636.2 | RP11-35J1.2 | AL031777.1 | Metazoa_SRP |
| LINC01348 | SCAANT1 | RP11-96C21.2 | Metazoa_SRP | AP002518.1 | SH3PXD2A-AS1 |
| RP11-275E15.3 | PCAT5 | AC133644.2 | AC137695.1 | AC090181.1 | RP13-465B17.5 |
| Metazoa_SRP | AL049822.1 | AC239868.2 | RP11-68E19.2 | AC009133.23 | LINC00628 |
| Metazoa_SRP | Metazoa_SRP | RP1-12G14.9 | RP4-777O23.3 | U91328.1 | AC239868.3 |
| LINC01127 | LINC01097 | AL009179.1 | LINC00997 | Metazoa_SRP | Metazoa_SRP |
| LINC00506 | Metazoa_SRP | BLACAT1 | TGFB2-OT1 | AC010168.1 | RP11-223J6.2 |
| DNAJB5-AS1 | LINC00538 | Metazoa_SRP | ERC2-IT1 | AC093373.1 | C2-AS1 |
| RP11-567F11.2 | AL031777.2 | Metazoa_SRP | Metazoa_SRP | RP11-439M15.1 | Metazoa_SRP |
| RP3-510D11.4 | CTB-127M13.1 | RP11-126O22.10 | RP11-126O22.9 | CTD-2336O2.3 | TMBIM4 |
| RP11-506F3.1 | CTA-286B10.8 | RP4-621F18.2 | CICP19 | HSP90AB4P | OR4G3P |
| RP11-27G14.4 | RP11-126O22.8 | CYP3A7-CYP3A51P | RP11-126O22.2 | RP11-126O22.4 | RP11-496N12.9 |
| RP5-1052M9.5 | FAM138F | CTA-286B10.7 | C11orf71 | CH17-31A6.1 | AC008993.3 |
| FRG1DP | AKR1C3 | RP11-307E17.11 | RP11-731J8.3 | LA16c-407A10.3 | BLOC1S5 |
| CTD-2195M15.4 | RHOXF1P3 | TRBV7-2 | RP11-683L23.7 | RP11-807C20.2 | KB-1396H2.2 |
| CDC27P3 | RP1-273N12.4 | RP11-176H8.3 | DUX4L37 | RN7SL1 | RP11-557N21.1 |
| FRG1FP | RP11-210L7.3 | RP11-417N10.5 | RP11-834C11.15 | CTD-2331H12.8 | ABC11-4932300O16.1 |
| RP11-964E11.3 | CTC-325H20.8 | RP11-107E5.4 | HYMAI | WASIR1 | IL3RA |
| CRLF2 | RP13-297E16.4 | RPL14P5 | LINC00685 | DDX11L16 | LINC00102 |
| RP13-465B17.5 | CLSTN3 | EIF3H | EDF1 | JMJD8 | ASPM |
| E2F4 | USP33 | FBXO9 | RP11-143J12.3 | AFF3 | CCBE1 |
| AC000120.7 | KPNA6 | GPR183 | CTD-2616J11.9 | SMAD2 | CDKN2B-AS_3 |
| PVT1_1 | COPS2 | LOXL3 | OFD1 | SCARNA8 | Y_RNA |
| SNORA1 | CSPG4P5 | BRS3 | KLF14 | NOTO | PLEKHS1 |
| PLA2G4E-AS1 | CTD-2525P14.5 | RP11-252I13.1 | IHH | NECAB1 | CTD-2647E9.1 |
| RP1-80N2.4 | RP1-257C22.2 | ARIH2P1 | EEF1A1P22 | ZNF415 | RPL4P3 |
| MYO5BP1 | ABCC5 | SRPK2P | GLYAT | SDC2 | HNRNPA1P37 |
| SEPT14P2 | OR52B3P | HMGN2P6 | OR7E37P | VN2R10P | PRPF38AP2 |
| TSPAN15 | DBF4P1 | IL1RAP | HIGD1AP14 | HBE1 | AC006116.27 |
| RP11-734K2.3 | YWHAZP6 | CACNA1C-AS4 | RP11-433P17.1 | AC010987.6 | CDC27P1 |
| RP11-390P2.4 | RP13-580B18.4 | RP11-756J15.2 | RN7SL466P | RP11-309L24.4 | PSMA2P3 |
| RP11-112J3.16 | HERC2 | IGHEP1 | RP11-434D2.9 | RP1-50O24.6 | ZNF354C |
| RPL15P2 | ERHP1 | RP11-92C4.4 | AC012363.4 | HMGB3P10 | RP11-286H14.4 |
| AC002064.7 | RPL34P34 | RP11-686G8.5 | CTD-3131K8.3 | RP11-322E11.6 | BCAS3 |
| TRAV14DV4 | TBX21 | AF196972.4 | MUC1 | CTD-2284J15.1 | RP11-276E17.2 |
| RP11-323F24.4 | PGAM1P10 | SEZ6 | GPR151 | RP11-542F9.1 | KCTD16 |
| ZNF330 | RP11-732A19.2 | RP11-523O18.1 | RP11-452L6.5 | CTC-251I16.1 | MAGT1 |
| NF1 | SERPING1 | U3 | RP11-475E11.9 | PRR4 | CTAGE15 |
| PP14571 | RADIL | PCDHGB2 | WASH6P | WASH6P | RP11-329N22.1 |
| ME2 | BHLHE41 | WBP1 | CFHR3 | RP4-694B14.8 | GPATCH1 |
| RP11-427L15.2 | MARCH7 | DUTP2 | GLO1 | SLC8A1 | RP11-466F5.3 |
| PRKAG1 | C1orf216 | RP5-1009N12.1 | ACAA1 | HNRNPA1P5 | CEP85L |
| AC003005.2 | RNU2-2P | TMPRSS6 | TMX2P1 | RP4-706A16.3 | RP11-76H14.2 |
| PRPSAP2 | RP11-164P12.5 | RP11-535A19.1 | NKPD1 | CLUL1 | SLC16A2 |
| RP11-667K14.8 | YWHAZ | PAX8-AS1 | G3BP1 | RP11-524F11.2 | RP11-247A12.7 |
| TMEM25 | RP3-380B8.4 | RP11-160A9.3 | RN7SKP160 | NTNG1 | RP11-133K1.9 |
| CTC-534A2.2 | GNB1L | S100A16 | CTC-246B18.10 | COX5BP6 | AATF |
| RN7SL77P | RP11-265B8.5 | S100Z | LDHAP2 | ACTR3 | RBBP7 |
| RP11-347P5.1 | C19orf84 | PRRT2 | TCF25 | RPS2P7 | NFATC1 |
| AC009005.2 | MORC3 | CKM | HIC2 | RP11-468O2.1 | SNORD22 |
| RP3-405J10.5 | CEP57L1P1 | PAK2 | RP11-12A20.6 | RP11-53OC5.1 | AF196972.9 |
| EIF1AX-AS1 | CFL2 | CALB1 | RP11-876N24.4 | NDUFB6 | SDHDP2 |
| C6orf163 | WNT2 | RP11-98D18.16 | LDHAL6A | DDX50P2 | IFITM3 |
| MAP2K5 | BAG2 | LAMC2 | RP4-593C16.4 | RP11-848P1.5 | CTD-3035D6.1 |
| LCMT2 | CYP3A4 | NT5DC4 | H2AFZP6 | ZDHHC20P4 | FOXD3 |
| DNAJA1P3 | RPL21P119 | HNRNPA3P1 | HLA-DQB1 | RPSAP76 | GPR45 |
| LAMB3 | RP11-756A22.7 | CCDC181 | RNF5 | RP11-316M21.6 | RALB |
| RP11-15B17.2 | P3H3 | RP11-429B14.1 | HOXC10 | SAMM50P1 | PPP1CB |
| RP11-323I15.2 | RP11-428C19.5 | SS18L2 | EXOSC4 | RP11-2K6.1 | GYS2 |
| VDAC2P3 | TENM1 | UBXN4 | TBC1D7 | RP11-507E23.1 | ANKRD49 |
| SMIM12 | RP11-71L14.4 | GLTP | RP5-1092A3.5 | RP11-317N8.4 | XXbac- |
| | | | | | B444P24.14 |
| AP003068.18 | RP3-414A15.2 | LDHBP2 | RP5-1050D4.3 | CCDC183 | USP5 |
| AC007249.3 | ABCF3 | RP11-671J11.4 | HNRNPKP2 | RP11-80I15.1 | ZCCHC11 |
| RPS21P1 | CYS1 | RGPD4 | RP11-284F21.9 | RP11-571M6.15 | LRRC69 |
| F5 | AF131215.1 | RAD18 | RP11-872D17.8 | GRPR | ST3GAL2 |
| RBBP8P1 | TACSTD2 | RP11-316O14.1 | RP11-638I2.10 | TBC1D3P1-DHX40P1 | RP11-326A19.3 |
| RP11-1070N10.5 | RUNX2 | SULT2A1 | PDE8B | ITIH4 | TG |
| MORF4L2-AS1 | GAS1 | STC2 | RN7SKP269 | RP11-440I14.2 | HSPD1P5 |
| RP11-503I22.2 | RP11-757G1.6 | RP3-467L1.4 | SEPHS1P4 | RPL18 | NOTUM |
| WWP1P1 | RP3-391O22.3 | CTD-2587H24.1 | CTA-384D8.36 | ASAP1-IT2 | NOXO1 |
| AL603831.1 | RP11-84C10.1 | THCAT158 | HARS | RP11-158M2.2 | RN7SKP150 |
| SYT5 | S100A11P2 | TERF1P4 | XXcos-LUCA16.1 | ANGPTL3 | GTF2IP5 |
| SHH | AC004057.1 | NEPNP | GDF7 | RPL21P75 | RP11-438L7.3 |
| RP11-329N15.3 | CEBPZ | CTA-150C2.20 | KCNC4-AS1 | AC079354.5 | SDC1 |
| RP11-250B2.3 | EIF5A2 | RP11-259O2.3 | PSMD5 | ATAD2 | RP11-17E4.1 |
| MARCH8 | RP11-405L18.4 | RP11-242D8.3 | RP11-723D22.3 | MAGEA6 | LINC00242 |
| CTC-499B15.7 | SCDP1 | RP11-1072C15.6 | VPS13A | PSME4 | RP11-1090M7.3 |
| KB-1460A1.1 | SERPIND1 | CENPUP2 | PHGR1 | LLNLF-173C4.2 | HMGN2P3 |
| FOXD1-AS1 | CLCP2 | AC007163.3 | KRIT1 | NPTN-IT1 | TGOLN2 |
| RP4-811H24.9 | RP11-582E3.4 | RP11-80H5.7 | MTCO1P31 | CTD-2073O6.1 | VASN |
| PTTG4P | RP11-498C9.16 | VWA5B2 | RP11-572N21.1 | HSFY3P | FBXO40 |
| RP11-96C23.13 | RP11-111J6.2 | DYNLL1 | MTRF1 | ZNF488 | SDC4 |
| AK3P5 | RP11-82O19.1 | ZNF962P | SPARC | RP11-820K3.3 | IL7 |
| RPL29P23 | RP11-13J8.1 | LINC00462 | RP11-541P9.3 | RP11-66B24.2 | AC004951.5 |
| SNRNP40 | OAS1 | AC159540.14 | RP11-770J1.7 | CASS4 | RP11-544O24.2 |
| CPNE7 | KNOP1 | RP13-638C3.5 | STX2 | PHKG2 | RP11-709D24.8 |
| RBM41 | AC007743.1 | FCN1 | HSPE1P4 | RP11-576I22.2 | CBX5P1 |
| RP11-303E16.5 | EGF | HECW2 | RP11-693J15.3 | RP11-323J4.1 | GTF2IP23 |
| RP11-379B8.1 | RP1-93H18.1 | ECM2 | RP11-1094M14.7 | SLC5A3 | RP11-426C22.1 |
| NAT8L | RP11-596D21.1 | CTA-363E6.7 | LL22NC03-32F9.1 | DPPA3P1 | XXbac- |
| | | | | | BPGBPG55C20.2 |
| STX12 | RP11-172E9.2 | YWHAEP5 | TMEM258 | RP11-244F12.2 | AP001610.5 |
| RN7SL130P | RP4-533D7.5 | CTB-12O2.1 | MIR762HG | SETP16 | EIF4BP3 |
| LYPLA2P1 | RPS4XP17 | RPL29P14 | GPR37L1 | FAXC | PEBP1P2 |
| RP11-1167A19.6 | RP5-864K19.7 | RP11-67L3.5 | RP11-45M22.5 | AC133633.1 | STX3 |
| P4HA3 | AC017002.2 | CTD-2313J17.7 | UMODL1-AS1 | GCM1 | OVOL3 |
| HRASLS2 | RP1-261G23.7 | C16orf58 | COL19A1 | RP11-567F11.1 | RP4-584D14.6 |
| RP1-63M2.5 | RP11-584P21.2 | MRPS27 | RAPGEF1 | MYH9 | RP11-258F1.1 |
| RP1-80N2.3 | RP11-356J5.12 | MAPK3 | WDR63 | Metazoa_SRP | RP11-973N13.4 |
| SMIM15 | ZSCAN26 | RP11-428G5.7 | CTC-429P9.1 | CTA-397H3.3 | RP11-603J24.14 |
| IGBP1P1 | RP11-50D9.4 | RPL7P46 | AC004237.1 | EPHB2 | AGT |
| CELF1 | CHCHD6 | RP11-109L13.1 | FZD4 | CTC-435M10.3 | CC2D2B |
| RP1-206D15.6 | NPM1P19 | RP11-445N20.2 | DR1 | SCARNA6 | LMNTD2 |
| CEP135 | RAI1 | RPS10P18 | FLT1 | BCL2L11 | AL021546.6 |
| RP11-495P10.7 | FAR1-IT1 | RP11-252O18.2 | TLE6 | MROH7-TTC4 | CTD-3028N15.1 |
| C16orf45 | ANKRD40 | RP11-514P8.2 | NPW | RP11-459O16.8 | SRSF12 |
| NDRG2 | SMPD1 | RAP1AP | RP3-414A15.11 | SH3BP4 | RP11-531A24.5 |
| AC100830.3 | RP11-1060G2.1 | KTN1-AS1 | FBXL3 | PRNCR1 | ZNF587B |
| RP11-573D15.2 | MCFD2 | AC110769.3 | ZNF702P | RP11-25L3.1 | RP11-303E16.7 |
| NIPAL4 | AC018766.6 | RPS15AP12 | RP11-334E6.10 | AC009120.10 | RP11-104H15.10 |
| EEF2 | Metazoa_SRP | CPE | COL28A1 | AADAC | RP5-1119A7.14 |
| VDAC2P2 | SPATS2L | USP8P1 | SERPINA11 | TXLNG | AC079466.1 |
| RP1-102H19.7 | STAU2-AS1 | YBX1 | MBOAT2 | GCOM2 | RPSAP13 |
| RP11-17J14.2 | RP11-405M12.4 | MIR193BHG | NAP1L4 | TGFA | RP11-274A11.3 |
| RN7SL851P | SLC25A2 | FAM221B | NACC2 | RP11-694I15.7 | CTD-2008P7.1 |
| FLNC | RP11-162A12.4 | AC096649.2 | MCM3 | CTD-2017C7.1 | C10orf67 |
| TUBA1B | RP11-171I2.3 | AC003682.16 | DDX59 | AC011747.6 | SNHG1 |
| NOP14 | CPS1 | CTB-32O4.2 | RP11-835E18.4 | RP13-487P22.1 | AFM |
| RP11-261P9.4 | HR | CTA-14H9.5 | SEMA6B | RN7SKP70 | RP11-111A21.1 |
| MCOLN3 | RP11-387H17.4 | SDAD1 | RP1-102K2.9 | RP11-727F15.13 | AC093110.3 |
| RP11-59H7.3 | RP11-6D1.3 | C16orf52 | R3HDM1 | RP11-34A14.3 | HDAC11-AS1 |
| DPY19L1P1 | LINC01234 | RP4-794I6.4 | RNF157 | RLN1 | RP11-326I19.2 |
| AC140725.4 | CTC-325J23.2 | AC021876.4 | AC253572.1 | RP11-557J10.4 | LDHBP1 |
| TMEM121 | PHLDA3 | PLVAP | PDXDC2P | LINC01400 | RPL7P9 |
| CTB-55O6.13 | LRRD1 | AC005154.8 | PIK3R1 | RP11-73M18.11 | HMGB1P14 |
| HTR3A | VSNL1 | RP11-84A19.4 | AL163953.3 | PSMA7 | RP11-697E22.2 |
| RP11-38G5.2 | TFG | TRIM11 | LMAN1L | PHBP12 | TAS2R18 |
| CNOT6L | IGFBP3 | EFNA1 | C20orf196 | AC006538.4 | CH507-9B2.4 |
| KCNE1 | AC012594.1 | AC079779.6 | IMPACT | PTPN21 | RBM38 |
| RP13-977J11.6 | CTD-2201E18.5 | GBP4 | RP11-303E16.8 | BHMG1 | CTBP2P7 |
| KLHL10 | MYOF | IGBP1 | ZKSCAN3 | CDC73 | ILKAP |
| RP11-351I24.1 | ACTG1P15 | IMPA1 | CABP7 | MIR3179-1 | RPS15AP38 |
| CTC-137K3.1 | SRGAP2 | RP11-723O4.6 | RP11-96D1.3 | RP11-182N22.8 | RP11-122G18.7 |
| CTC-756D1.3 | AC009060.2 | CHRM5 | ANO10 | MFHAS1 | ABCC2 |
| LL22NC03-24A12.9 | DNAJA2 | OPHN1 | CCDC58 | SERPINE2 | NDEL1 |
| RP11-16E23.4 | FAM234A | RP4-593H12.1 | ZFP69B | RP11-9E13.4 | LA16c-OS12.2 |
| RP11-426C22.5 | CELF2-AS1 | CTA-253N17.1 | CCDC106 | RP11-493L12.4 | AC009948.7 |
| HMGB1P8 | HECA | UBE2J2 | PCNPP5 | TRAPPC8 | FCGR2A |
| PITPNM3 | YPEL5P2 | FDPSP8 | IDNK | RP11-454L9.2 | RP11-602N24.3 |
| CYP2F1 | EWSR1 | RABGAP1L-IT1 | RP11-169E6.1 | RPL14P3 | UBA52P8 |
| KCNV2 | ADSL | RP11-46F15.2 | RBMX2 | RP11-478C6.6 | PWAR1 |
| ARAP1-AS2 | RP11-214K3.5 | NID2 | RP11-45A17.3 | HSFX1 | GAS2L3 |
| PHACTR3 | RPS17 | PIK3R4 | AC083843.2 | KIZ-AS1 | SARNP |
| DES | RP11-400N9.1 | CRKL | ULK4P2 | CTB-83J4.1 | RP1-253P7.4 |
| RP11-39C10.1 | GREB1L | HGD | LINC01126 | RPL13P6 | MIA-RAB4B |
| SYPL2 | KRT18P31 | SP6 | GPHA2 | CYP4A22 | E2F6P1 |
| RENBP | ACSM2B | SHCBP1 | GRPEL2-AS1 | RP11-262H14.3 | TBC1D8 |
| RPL14P1 | LRRC19 | RP11-120K18.2 | GAPDHP2 | CTD-2619J13.27 | CHRNA4 |
| RNF152 | RP11-109A6.3 | RP11-516A11.1 | TRAPPC13 | MT1G | TCTE1 |
| RP11-66N5.2 | WDR26 | AC005082.12 | CTD-2515A14.1 | BIVM | ADCK1 |
| RP11-299G20.5 | PEBP1 | CTD-2012K14.8 | STRN | AC009950.2 | LLNLR-304A6.1 |
| EEF1A1P10 | TRMT61B | PPP1R37 | GPR52 | DOLPP1 | CTD-2349P21.6 |
| RP11-317P15.4 | HADHB | RP11-51J9.6 | RPS6 | TNFSF13B | RN7SL535P |
| FBLIM1 | CD274 | RP11-84C13.2 | RP1-288H2.2 | SLC25A14P1 | AC009245.3 |
| EOMES | RP11-395N3.1 | SLC22A13 | CDC42EP4 | CLDN9 | GAS6 |
| CENPCP1 | SLC9A5 | AC003104.1 | GAPVD1 | RP11-1250I15.2 | AK4P3 |
| PSMG2 | KLF1 | AC099668.5 | C11orf65 | CFHR5 | AC006160.5 |
| MPRIP | KIAA1468 | UNC80 | PREX1 | DNAH14 | YWHAH |
| NFE2L3P1 | RP11-500B12.1 | ASB13 | CTD-2583A14.9 | ELFN1 | RP11-45A17.2 |
| ENPP7P8 | SMAD3 | FAM206A | ZNF554 | RP4-733B9.1 | RP11-528I4.2 |
| TEX15 | RP11-121C6.4 | ARHGAP26 | TCERG1 | RP11-395A13.2 | U2SURP |
| TTC14 | GJC2 | PCDHGB9P | FANCD2OS | MBOAT4 | CDV3 |
| EIF2S3 | CCDC53 | RP11-344P13.3 | WBP4 | HIGD1B | TNPO3 |
| FAM228B | ACVR2B-AS1 | ACSL4 | RPL41P2 | PARK7 | GOLGA8T |
| RP11-617B3.2 | RP11-427J23.1 | RP4-785G19.5 | RP11-767C1.2 | KCNS3 | FAM187A |
| TOB2 | WNT7B | NEDD1 | ATMIN | RP11-3D4.3 | RP11-101E7.2 |
| IFIT1P1 | BLOC1S4 | RP11-266L9.6 | RFESD | RP1-278E11.3 | MORN4 |
| KCTD3 | LINC01088 | ZNF705A | KCNS1 | RPL4 | POLA1 |
| RP11-452H21.4 | RPL36 | CDRT1 | BAIAP2 | URGCP-MRPS24 | RP11-166B2.5 |
| PCDHGA3 | SLC30A3 | RP11-50D9.3 | RP11-692D12.1 | WASF3 | RPL30 |
| FOXK1 | NENFP1 | THOC7 | ABCE1 | SEC24C | AC005534.8 |
| HSP90AB2P | MBP | RP5-1050D4.4 | ZMYND19P1 | CHRM4 | RP11-523H24.3 |
| ARNT2 | DHX57 | RP11-307P22.1 | SERPINA4 | FOXA3 | AC023491.1 |
| AC007325.3 | RP11-111H13.1 | CDKN2A-AS1 | RP4-669L17.8 | ACE2 | FBXO21 |
| CARD9 | COMMD6 | COASY | ZNF622 | XXbac- | MUC20 |
| | | | | BPG246D15.9 | |
| RP11-135A1.2 | ZNF714 | MTATP6P1 | HOXC6 | GAPDHP48 | RP11-687F6.5 |
| CTB-102L5.7 | KPNB1 | RP11-304L19.3 | RP11-471B22.2 | RP11-560A15.6 | NUPR1 |
| MARK2P9 | AC105052.2 | ZNF320 | HMGB1P19 | RP11-295I5.3 | RP11-155O18.6 |
| MAPRE1 | MLK7-AS1 | NPM1P26 | RP11-277P12.6 | IGLVIVOR22-1 | EI24P2 |
| RP11-305K5.1 | ZCCHC14 | RTN3P1 | RP11-59C5.3 | ZFAS1 | DIS3 |
| RP11-498C9.4 | RP3-426I6.2 | MOXD1 | AC009133.12 | TDH | RHOBTB2 |
| DNAJC11 | RP5-981O7.2 | OSR1 | COX6B1 | RP11-61L19.1 | TRIM45 |
| ADRBK2 | PLCL1 | MAN1A1 | IQCA1 | MIR3180-2 | MIR3180-3 |
| HPCAL1 | RP11-571F15.3 | CSNK1G2-AS1 | RPL12P6 | AC093642.3 | U52111.14 |
| RP11-357N13.6 | GS1-166A23.2 | RP11-973H7.3 | RP11-141M1.3 | RP11-488P3.1 | ADAMTS7 |
| RNF34 | SRRM1P3 | GATSL2 | ZNF17 | RP11-277P12.9 | RP11-37B2.1 |
| AZGP1 | RP11-339B21.10 | AL109927.1 | CYP21A2 | RP11-873E20.1 | AC010642.2 |
| ISOC2 | CTD-3037G24.4 | CD163L1 | ZSCAN12 | NRARP | LMOD3 |
| AP003419.11 | RP11-90H3.1 | NDUFS1 | BTN2A2 | SF3B6 | GABRP |
| AC004540.5 | PCED1B | HIST1H3PS1 | EIF5A | RUNDC3A-AS1 | ANKRD20A3 |
| UROD | AGA | IGLV1-51 | TYW1B | TRPM7 | LRRC24 |
| THAP11 | AQP3 | HOTAIRM1 | VAV1 | AC001226.7 | CCDC144B |
| DNAJC9 | RP11-369C8.1 | RPL31P52 | SCRT1 | RP11-380N8.7 | TIMM23B |
| FTH1P8 | RPL7P23 | UBA52 | PROSER2-AS1 | ST13P19 | RP11-736N17.4 |
| OTUB2 | GDPD5 | NDST2 | CTA-268H5.14 | ZNF471 | RP11-635N19.1 |
| RAMP2 | LINC00264 | WDR55 | SERPINA6 | PCDHA10 | RP13-270P17.3 |
| CSE1L-AS1 | ADIRF | CTSE | RP11-712B9.2 | RP11-677M14.6 | AGO2 |
| RP11-244N20.7 | PSMD13 | RP11-384P7.7 | NR5A1 | DENND6A | LINC00907 |
| GVQW1 | CCDC13 | FPR3 | AC009303.1 | RP11-261C10.7 | GUSBP5 |
| LINC01036 | ZNF593 | RP11-1191J2.2 | RAB17 | CLPX | CTD-2542C24.5 |
| DCAF11 | MAPK6PS3 | RP11-517C16.2 | RP3-522J7.7 | CTD-2516F10.2 | LINC00857 |
| RP5-1142A6.2 | RPP14 | MUC19 | NSD1 | RP5-1049G16.4 | G3BP2 |
| GALNT13 | KLF2P1 | ATXN7 | SYTL1 | DYRK2 | RP11-647K16.1 |
| AC104451.2 | NDUFAB1 | GBP3 | MPND | LYG1 | RP3-465N24.6 |
| RPS4Y1 | DAPK1 | TMCO3 | SSC4D | DCTN3 | KB-1572G7.3 |
| MFSD1P1 | AC007365.3 | RP11-536C10.12 | CH17-262A2.1 | TRGV3 | HSPA8P5 |
| SORD | ATE1-AS1 | ZMYND15 | RP11-188P20.3 | GPC2 | PHRF1 |
| CTB-129O4.1 | BANF1P3 | DDX42 | RP11-468E2.2 | FAM193B | RPL36A-HNRNPH2 |
| ZCCHC17 | RN7SL273P | RP11-550F7.1 | HNRNPCP1 | MSN | RP11-126L15.4 |
| UTP6 | CRLF3 | YWHABP2 | CEACAMP3 | UBE2V1P2 | RP11-295K2.3 |
| ARHGAP31 | RBMS3-AS2 | FBXO34 | SEPSECS-AS1 | VNN3 | DENND1C |
| WHSC1L1 | PRR14 | LFNG | RPAP3 | CTA-398F10.2 | ACRV1 |
| LINC01270 | ROMO1 | TOX3 | HIATL2 | MYO1A | NLRP4 |
| POLN | P3H4 | RP11-473M20.16 | JMJD1C | LL22NC03-23C6.13 | DAB2 |
| RP11-421F16.3 | MYADML2 | LYPLA2 | CCDC40 | MED26 | IPO4 |
| LINC00365 | MCAM | ZNF141 | RP5-884G6.2 | FOLR2 | CTB-50L17.7 |
| RP11-496I2.5 | AC026271.5 | ATP1B3 | LTBR | MGC39584 | SUMF2 |
| RP11-384C4.2 | DRC1 | APOC1 | THRAP3 | RP11-505P4.6 | PKD2 |
| PRKCA-AS1 | RP11-244H3.4 | THUMPD1 | NPM1P27 | PRKG1 | RP11-24I21.1 |
| NUP98 | RP11-252I14.2 | MPRIPP1 | PLAC4 | CTD-2006C1.12 | CITF22-92A6.1 |
| XKR6 | RP11-843P14.2 | RP11-199F11.2 | SERF1A | ZNF770 | ZNF792 |
| DUTP6 | HLA-V | RP4-564F22.7 | RP1-102E24.9 | RUSC2 | HMHA1 |
| C16orf72 | RP11-149F8.3 | KIF5B | IGHV2-70 | CCL27 | PSMC2 |
| HBA2 | RP11-378A13.1 | RANBP2 | RP11-248B24.1 | RP11-1398P2.1 | DCDC2B |
| MAK16 | RMI1 | TRIM33 | RN7SL263P | LA16c-360H6.2 | CHD1 |
| RP11-667M19.5 | BMP7 | SERPINA3 | ANKRD31 | CTD-2026K11.4 | TMEM115 |
| CLIC4P3 | OGN | DIDO1 | RP11-89F3.2 | ANKRD13D | WFIKKN2 |
| RP11-383G6.3 | AC009014.3 | RP4-695O20.1 | AC003005.4 | PABPC1P3 | PDLIM2 |
| RP11-475E11.2 | GJB2 | RP13-20L14.6 | RP11-255H23.4 | PEX7 | RP11-26J3.3 |
| NEDD9 | RP11-349N19.2 | GMEB2 | RP1-212P9.3 | HKDC1 | EDA2R |
| HDLBP | ZNF639 | IGLV2-5 | RP5-1142J19.2 | NLRC5 | PRSS33 |
| FNIP1 | RPL34P17 | DNMT1 | DONSON | RP11-1348G14.5 | NHS |
| MEIS3 | RP1-232L22_B.1 | SOX15 | HIGD2A | ARF4P2 | TMEM56 |
| SMIM6 | CLTB | CTD-2213F21.2 | GTF2I | LMF1-AS1 | IFNA20P |
| ZNF497 | TGM2 | C8orf37-AS1 | RP5-1125A11.6 | COG6 | MT-ATP8 |
| IL10 | SPDYE16 | WNK1 | AC006372.4 | FPGS | RP11-798G7.5 |
| YTHDC2 | AC004221.2 | ATAD5 | LENG8-AS1 | EEF1G | ADH5P4 |
| MTRR | AEBP2 | MRPS33P2 | CSPG4 | AC007161.5 | RP11-616K22.1 |
| LINC01419 | TFF1 | LINC00158 | RP4-665J23.1 | RP11-685A21.1 | HBA1 |
| INHBC | TTI2 | YAE1D1 | MRPL3 | RP11-1376P16.2 | RP11-275I14.4 |
| DPRXP4 | SNHG11 | NAA25 | TCEB1 | RP4-539M6.21 | FKBP15 |
| RP11-423H2.3 | COX5BP3 | RP11-443P15.2 | AC002075.4 | CPEB2-AS1 | VIM |
| GPR35 | RAVER1 | ZNF853 | RP11-126K15.1 | GS1-124K5.13 | RP11-793I11.1 |
| RAB5CP1 | AP000640.10 | CREB3L1 | ALOX15B | AC007292.7 | COL14A1 |
| KIDINS220 | AC005786.3 | RP1-65P5.3 | ANAPC5 | Z98744.1 | RP11-146E13.5 |
| RP11-326C3.7 | ZNF148 | STEAP1 | KCNH6 | GPRC5C | CHRAC1 |
| RPL39P36 | SNORA5C | GLIS3 | AC000041.8 | STIM2 | RP11-296K13.4 |
| SNORA48 | uc_338 | ABCA11P | SEPT14P4 | RP11-295D4.7 | RNU2-11P |
| ABHD15-AS1 | SDS | POMP | FGD5 | CTD-2349P21.7 | RP11-304M2.6 |
| RP11-12A20.10 | AC074117.13 | ZAR1L | ZFP69 | AFF4 | RP11-4M23.3 |
| HHIPL1 | U3 | RP13-582O9.7 | CTC-204F22.1 | RP11-455O6.5 | HSP90AA2P |
| RP11-85B7.5 | SOX2OT_exon1 | RP11-24J19.1 | AC005838.2 | C1QA | TMEM127 |
| SNORA45A | SNORA2 | SNORA49 | RP3-365I19.2 | CH17-232I21.1 | MIR1248 |
| BRCA2 | RP11-792A8.3 | FAM155A | KIAA1644 | ABCA13 | CDC42BPA |
| RP11-66B24.7 | CD24P4 | CTD-2311M21.3 | ANGPTL1 | SOX9 | ANKRD20A8P |
| SPATA31C2 | KLHL34 | RIMS1 | RP5-998N21.4 | GPR88 | LINGO4 |
| BEND2 | FAM83C | GLP1R | RP11-712C19.1 | PARP1P1 | SNX19P2 |
| NR2F2 | TARID | GNG4 | INSM1 | ABCB11 | FOXI3 |
| CXCL9 | TNPO1P3 | FER1L5 | ICOS | KCNA4 | ERVMER34-1 |
| CXCR1 | CTAGE10P | CTAGE11P | CICP2 | CLEC14A | RP11-84A19.2 |
| RP11-522N14.2 | LINC00839 | SLC9A3P3 | TRHDE | RP11-742D12.2 | CEBPA-AS1 |
| TMEM78 | AC092535.3 | MAPK6PS4 | RP11-305A4.3 | NOX3 | FAM66B |
| SDAD1P2 | SULT4A1 | C3orf80 | RP11-389G6.3 | LAMA4 | SPRY4 |
| RP11-331G2.6 | NDN | MICAL2 | KB-176G8.1 | RP1-136J15.5 | ELN |
| RP11-190D6.1 | RP11-561O4.1 | RP11-1216L17.1 | MAATS1 | RP11-769O8.1 | TPD52L3 |
| LINC01018 | KLRG2 | GSX1 | TIMP4 | RP11-185E8.2 | CYLC1 |
| ZFP2 | CTD-2535L24.2 | RPL23AP92 | RP11-59D5_B.2 | RP11-26J3.1 | PLA1A |
| AF064858.8 | RP11-420O16.2 | LL22NC03-84E4.11 | PSMD12P | BTBD10P1 | CTD-2126E3.5 |
| EPO | CTD-2126E3.3 | ADAD1 | CTD-2651B20.1 | RBBP4P5 | PPIC |
| ACTR3P2 | RP11-350G24.2 | IL1RN | RP11-324E6.6 | RP11-211N11.5 | FLJ22447 |
| RPL21P132 | HAO2 | ANXA3 | RP11-804M7.1 | CEACAMP10 | RAB32 |
| EIF4A1P9 | KCTD9P3 | KCTD9P1 | RP11-259P15.4 | POLR3GP1 | NAP1L4P3 |
| DSPP | RP11-483G21.3 | RP11-288C17.1 | CTC-512J14.7 | HNRNPA1P26 | REP15 |
| FDPSP1 | PRODH2 | HMGA1P1 | CTD-2331H12.5 | ZNF41 | TCP10L2 |
| RAET1G | LINC00941 | GAPDHP44 | RP11-473M10.3 | HCG4P11 | AC104389.28 |
| RP11-510N19.5 | RP11-761B3.1 | AC073316.1 | HNRNPA3P7 | HMGN1P30 | PLAC1 |
| RBMX2P5 | AC068491.3 | HSD17B1P1 | MTND1P8 | SMARCA5-AS1 | OR4K7P |
| CTD-2571L23.9 | WDR82P1 | FRG1JP | SPINK1 | ESRRAP2 | RP11-364P2.2 |
| LINC01320 | CNTNAP3P1 | SLC25A5P2 | SDCBPP3 | SDCBPP2 | RP11-388B24.4 |
| RP11-183G22.1 | GPR79 | NPM1P35 | RP11-268P4.4 | RP11-346C16.2 | AC064852.4 |
| VDAC3P1 | RP13-20L14.2 | IGKV1OR9-2 | XXbac- | BMS1P8 | RP11-477J21.6 |
| | | | B476C20.10 | | |
| RP1-202O8.3 | LINC01169 | MTND1P10 | RP11-411B10.4 | PEX5L-AS1 | CT45A10 |
| ATP1B3P1 | RPL37AP1 | CTB-20D2.1 | CTD-2528A14.3 | RP11-452K12.4 | RP11-367H1.1 |
| VGLL1 | RN7SL221P | RN7SL605P | RP1-164L12.1 | RPS4Y2 | AC007204.2 |
| AC069363.1 | RPS4XP22 | RP11-778J16.2 | AJ271736.10 | AJ271736.10 | RPS4XP11 |
| RP11-462L8.1 | CDC42EP3P1 | RBP7 | RP1-302G2.5 | RP11-197K6.1 | RN7SKP266 |
| RPL34P21 | RP11-467L19.8 | RP11-109N23.6 | TMEM31 | RP11-63E16.1 | RN7SL502P |
| RP11-982M15.5 | RPL7P34 | SNCG | RP13-714J12.1 | RN7SKP57 | GTF2IP2 |
| CLYBL-AS2 | BCRP1 | RP3-412A9.15 | AP000282.2 | RP11-650K20.3 | RP11-486P11.1 |
| RP11-458F8.1 | RN7SL799P | MRPS36P4 | ZNF444P1 | SMR3A | RN7SL745P |
| RP11-365H23.1 | RP11-556O5.6 | SNHG10 | RP11-174G6.1 | RP11-478H16.1 | RP11-256L6.2 |
| Metazoa_SRP | AC016722.3 | RP1-310O13.13 | RP11-219G10.1 | AC010884.1 | RN7SL128P |
| RP11-627J17.1 | AK4P5 | PEX5L-AS2 | RP11-195C7.2 | RP11-686D22.7 | LLPH-AS1 |
| LDHAP5 | RP11-325L12.7 | RPL9P32 | RP11-344P13.4 | CTB-70D19.1 | CTD-2595P9.4 |
| CTD-3110H11.1 | RP1-102G20.5 | RP11-736E3.1 | RP1-273G13.2 | RP11-90D4.3 | IGHA1 |
| CTD-2583A14.1 | HMGB3P6 | HMGN1P35 | RP11-411G7.2 | RP11-599B13.8 | RPL19P21 |
| RP11-669B18.1 | RP11-35J10.5 | CTD-2207O23.12 | H2AFB1 | Metazoa_SRP | RN7SKP292 |
| NUDT19P4 | RP11-527F13.1 | HOXC-AS1 | AC011933.2 | RP11-93O14.2 | RP4-612C19.1 |
| RP5-1018K9.1 | AP001464.4 | IGKV2-23 | AC004837.3 | AF127577.10 | RP11-483P21.2 |
| RP5-940J5.8 | RP11-325K19.1 | IGKV1-22 | AC007272.3 | RP11-407A16.3 | RP1-27K12.4 |
| RP11-19J5.2 | RP11-573D15.1 | CTD-2568A17.1 | RPL17P46 | RP3-343K2.4 | CTD-2358C21.3 |
| RP11-34P1.2 | COX8C | RP11-10N23.5 | RPS12P27 | HOTTIP_3 | Z69890.1 |
| CTD-2105E13.16 | VPS13A-AS1 | RPL12P21 | MTND3P10 | CTD-2538C1.3 | CTB-102L5.4 |
| PTMAP4 | AL450992.2 | AC015936.3 | RP11-232D9.1 | NCOA7-AS1 | RP11-288E14.2 |
| ST6GALNAC4P1 | RP11-74C1.2 | FAM83C-AS1 | RP11-16F15.4 | RP11-474I11.8 | CFL1P6 |
| LINC01545 | RP11-301G21.1 | RP11-203M5.8 | RP11-95I19.2 | RPL21P134 | RP11-672L10.6 |
| RPSAP14 | RP11-254G11.1 | AF213884.2 | RP11-49K24.3 | AC006373.1 | AC006116.14 |
| RP11-493L12.6 | RP11-813P10.2 | RP11-387D10.4 | PPIAP32 | RPS27AP1 | GTSCR1 |
| RP11-611O2.2 | RP11-542G1.1 | RP11-316M1.3 | RP11-476B13.2 | EIF5AP4 | RP11-874G11.1 |
| CTD-2609K8.3 | CTD-2288F12.1 | CTD-2538G9.5 | CTC-459M5.1 | RP11-554E23.4 | HOTTIP_2 |
| PTRH2 | A2ML1-AS1 | RP11-508M8.1 | IGKV1-27 | RP11-194G10.3 | CTD-2529O21.1 |
| CTB-52I2.7 | LINC00555 | IGLV1-62 | RPS15AP6 | RP11-613E4.4 | RP11-476H20.1 |
| RPS24P18 | CCDC58P5 | AC008074.5 | RP11-49O14.3 | RP11-243M5.1 | AC015849.12 |
| ZDHHC20P1 | RP13-444K19.1 | RP11-21M24.3 | PRR13P2 | RP3-333H23.9 | RP11-54O7.14 |
| CTD-2287O16.3 | AC011290.4 | CTD-2623N2.5 | RP13-49I15.6 | DPPA2 | RN7SL176P |
| RPL23AP1 | LINC01123 | SYNJ2BP-COX16 | IL15RA | RP11-327P2.5 | RPL35A |
| PRPF4B | RPS27A | RN7SL573P | C19orf53 | POPDC2 | LHX6 |
| RP11-175O19.4 | bP-21264C1.1 | PRPS1 | CSNK2B | PTGIS | TMEM116 |
| RP5-875H18.4 | ANXA7 | TRAPPC6A | RP11-274E7.2 | UROC1 | IL17RD |
| OLA1 | TRHR | AC068831.3 | IGHVII-30-1 | SUV39H1 | TMEM27 |
| BLOC1S5 | AP001062.8 | CTC-548K16.2 | PCDHGB7 | RP11-863K10.7 | FABP12 |
| LA16c-425C2.1 | RNASEH2B-AS1 | GID8 | ZNF358 | OPLAH | PAIP1 |
| RP11-444E17.6 | STAB2 | CPD | UBXN10 | OR2D2 | TSEN2 |
| ZNF192P1 | DNAJC2 | CD4 | FAM131C | TBC1D3K | IFNLR1 |
| UQCRQ | RP11-452L6.6 | SYMPK | RASSF8 | KDM4C | RP11-123K3.4 |
| RP11-717K11.2 | RP11-161M6.6 | AC009237.8 | OSBPL10-AS1 | RP11-196B3.1 | MMP14 |
| GLUD1P3 | JPH4 | RP11-1000B6.5 | MKNK1-AS1 | RP11-287D1.3 | CHODL |
| ASXL1 | TSPAN14 | USP32P2 | FCER2 | XPO5 | PLAGL2 |
| SLC5A2 | ATAD2B | SSSCA1 | GNAL | BRWD3 | RHOB |
| WNT9A | ARAP3 | CD1B | GNLY | ENO1-AS1 | AC105393.2 |
| ARMC10P1 | RP11-1334A24.5 | RP13-638C3.3 | XPNPEP2 | RASSF4 | PMS2P9 |
| KLF11 | PLCG1-AS1 | RP1-232P20.1 | MVD | RP11-457M11.7 | AC002128.5 |
| OXCT2P1 | RP11-3D23.1 | RP11-1143G9.5 | RP11-59H7.4 | KBTBD6 | ORM1 |
| RP5-1171I10.5 | RDH10-AS1 | ODCP | RP11-567P19.1 | LL09NC01-139C3.1 | RP11-170M17.2 |
| PER1 | DRAM1 | WDHD1 | RP11-165N19.2 | IGKV1-8 | CTD-3193O13.8 |
| RP11-148K1.10 | RP11-661O13.1 | FTO-IT1 | ACTG2 | SPATA2L | EFCAB8 |
| ZBTB6 | RP11-73M18.7 | CLLU1OS | NXPH3 | TMA16 | UCK1 |
| C9orf173-AS1 | RP1-40E16.12 | KRT19 | KPNA3 | LINC00664 | SLA2 |
| VAMP5 | MCUR1 | RP3-496C20.1 | PRELID1P4 | RPL23AP95 | TPP2 |
| ASB7 | RP11-214K3.19 | TSPO | PTTG2 | CHML | PIPOX |
| CHMP1A | TFF2 | PCSK4 | CTD-3074O7.11 | PRSS36 | PHF12 |
| RHNO1 | HIST1H2BI | HSP90AA4P | MYO7A | RP4-605O3.4 | Z95114.7 |
| ADAMTSL5 | NBR1 | AJUBA | AD000684.2 | DDI2 | GSTA4 |
| ACSM2A | ZNF165 | TTI1 | DDX18P5 | CTA-38K21.2 | XIAP |
| RBMXL2 | RP11-142A22.3 | B3GALNT1 | MICD | CH507-513H4.5 | MTHFD2 |
| PLEKHN1 | CTAGE5 | MON1B | RP11-770E5.1 | LINC01002 | UBTF |
| RP11-293I14.2 | AC125232.1 | ZNF254 | RP5-1039K5.13 | IGKV1D-22 | MANSC1 |
| SNORD3A | SNRK | CTD-2369P2.8 | HMGB1P31 | TMEM55B | ZHX1 |
| H2AFY | COL1A2 | AC092580.3 | SYNRG | ZNF737 | RP11-235E17.3 |
| RP11-45M22.3 | IFNNP1 | RP11-129M16.4 | RP11-196B3.4 | GMPR | AARSD1 |
| PLK5 | RP11-121C6.5 | RP11-248J18.3 | RP13-36G14.4 | TNFAIP8L1 | RP11-535A19.2 |
| LINC00632 | RASA1 | PSENEN | RP11-649E7.7 | CTA-212A2.1 | AC005220.3 |
| AC092295.4 | PSMD6 | RP4-613A2.1 | C11orf72 | LCP2 | UBA5 |
| RP11-153F10.2 | RP11-196G11.6 | RP11-438L19.1 | RP11-417P24.1 | KCTD14 | TPI1 |
| SULT1C2 | RPS3AP26 | QRICH1 | PSMD8 | CTD-2026D20.2 | GYPC |
| ESPNP | RP11-468E2.6 | SLC52A2 | NCKIPSD | PM20D1 | GBAS |
| AC017101.10 | CTC-276P9.4 | RP11-755O11.2 | RP11-161I2.1 | HAUS4 | KRTAP5-9 |
| MED28P1 | SNORD15B | DLG3-AS1 | DOCK11 | SLC20A1 | RP11-269M20.3 |
| FAM96B | RP11-196B3.2 | MID1IP1 | CEP57L1 | CTB-134H23.3 | CFAP47 |
| RP11-6O2.3 | CICP8 | RP11-428P16.2 | SPZ1 | C17orf50 | LIPI |
| AC092620.2 | TASP1 | RPL37A | RP11-298C3.2 | RP11-848P1.3 | PPIA |
| RP11-403I13.7 | NPPA | AC104297.1 | RP11-419I17.1 | MTX1P1 | C10orf25 |
| AF015262.2 | RP11-132A1.4 | KCNMB3P1 | CALCRL | IBTK | ABTB1 |
| RPS24P8 | RP11-762H8.4 | RP11-384K6.6 | VRK3 | PPID | RP13-314C10.5 |
| ING2 | DNAJB13 | PRKRIRP7 | IL5 | RP11-195F19.30 | IMPDH1P10 |
| CSRP2BP | PGAM1P7 | RP11-235E17.5 | TXNDC16 | AC026248.1 | TRUB2 |
| RP11-362F19.1 | NUP205 | AC004019.13 | RP11-521C20.2 | DLX2 | GTPBP4 |
| CTD-3148110.9 | GAPDHP42 | RPL5P29 | SSSCA1-AS1 | CTC-543D15.1 | LINC00346 |
| CTAGE1 | LINC01220 | LAGE3 | JMJD7 | NHSL2 | AC097724.3 |
| FRGCA | CAPS | UVRAG | HEATR9 | SLC2A6 | CREM |
| RN7SKP287 | MED29 | CTB-33G10.6 | ERRFI1 | C11orf52 | AGAP11 |
| RN7SL657P | NFKBIL1 | TPO | ART4 | TXNDC9 | RP11-452H21.1 |
| APITD1 | GSDMC | MIR3681HG | CTD-3222D19.2 | RP11-398A8.5 | TYW1 |
| FAM133DP | CSNK1A1P1 | CFAP54 | PSMB3 | MAP3K3 | RNASE1 |
| ANKRD53 | RP11-680A11.5 | KRAS | PGM5P2 | FAM71E1 | GGTA1P |
| AKIRIN1 | LINC00384 | LILRB2 | LLNLR-268E12.1 | RAD17 | RP11-783K16.14 |
| HIPK1-AS1 | UBE2MP1 | PDE4DIPP1 | ZCCHC24 | SRSF9 | TEX2 |
| RP11-348N5.7 | HIST1H1D | NLE1 | SGSM3 | FBXO33 | AFF2-IT1 |
| DDX50P1 | FNBP1P1 | GNL3L | TAF10 | RP11-418H16.1 | AKAP1 |
| NME1 | AC137934.1 | VNN1 | HSD11B1 | TPCN2 | PLA2G2A |
| UBE2Z | AMDHD1 | PDP2 | APOC4 | AC015849.14 | POLR3A |
| RP11-432B6.1 | CDK16 | LINC00698 | LA16c-431H6.7 | RP11-791G16.2 | SDHAP3 |
| GLT8D1 | FTCD | POM121 | SLC35A5 | EFTUD2 | TMEM262 |
| LGALS9B | TMEM54 | YBX1P4 | AKAP6 | SF3A1 | ALOXE3 |
| SLC19A1 | CNPPD1 | MRPL37P1 | LA16c-313D11.9 | RP11-690I21.3 | MAPKAP1 |
| ZSCAN18 | SERTAD3 | RP11-1007O24.2 | PYCR2 | HPX | SLC12A1 |
| F10 | BCL2L14 | RP11-123K3.9 | RASSF3 | RP11-697E2.11 | SNHG12 |
| RP11-465N4.4 | EDEM1 | RPL7AP31 | PRKAR1B | DANCR | VTN |
| GPX1P1 | RP11-425L10.1 | GDNF-AS1 | RP11-498C9.2 | ERCC8 | PRMT1 |
| EIF3M | CTNNA3 | NFRKB | CEACAM6 | CTC-250P20.2 | PAPPA2 |
| SPATS2 | AC092597.3 | SENP2 | RP4-778K6.3 | DCTN6 | STK24-AS1 |
| URB2 | COX6CP1 | MED27 | TMPRSS4 | bP-21264C1.2 | RP11-624C23.1 |
| C11orf57 | RQCD1 | RP11-629N8.4 | EXOSC9 | RPLP1 | FBXO31 |
| CCDC109B | EPHA3 | C4BPA | ANKMY2 | PI4KA | OXER1 |
| RP11-486A14.2 | MLXIP | RP5-884M6.1 | RPL3P4 | RP11-829H16.3 | DDX39A |
| RP11-770J1.4 | RSRC1 | IL11 | RP11-371I20.2 | CARF | GRIK1 |
| LINC01341 | NCOA7 | RNASEH1 | SLC2A12 | CHST9 | CYP4A11 |
| WDR88 | TATDN2 | POFUT1 | RRP36 | RP11-333J10.3 | SNHG22 |
| RIMBP3C | FEZ2 | DHX33 | WASH5P | RN7SL425P | RP11-341G23.4 |
| CLDN5 | PIWIL2 | GCK | RP11-425M5.7 | SNX18P8 | C5orf63 |
| LINC00880 | NFAM1 | POP5 | SYNPO2L | AGXT | RP11-267N12.1 |
| RP11-493E12.1 | SYP | PEG3 | LARP4 | RP11-762I7.4 | RANP1 |
| AC137932.6 | MYO1B | LINC01481 | ZNF324 | USP42 | RP4-725K1.1 |
| ASB16 | RP11-264B17.2 | RPS10P3 | CTD-2358C21.4 | RP4-800M22.1 | EXOC3-AS1 |
| LTC4S | RP3-342P20.2 | LYPD6 | DLG2 | TTC27 | CSMD1 |
| RP11-1008C21.1 | RPA4 | CSNK1G1 | PDZD3 | RP3-368A4.6 | RP11-644F5.11 |
| ZNF283 | CTC-336P14.1 | RP11-875O11.3 | RP1-199J3.6 | RP13-977J11.8 | AKAP13 |
| RPL5P1 | KCMF1 | CACYBPP3 | CTB-75G16.3 | RP11-285E9.6 | EXOC8 |
| LA16c-380H5.6 | FAM126B | RP11-804A23.2 | RP11-864J10.2 | UBE2V1 | SLC34A1 |
| M1AP | RP11-295I5.4 | SYCP2L | AC093107.7 | CICP9 | RAB33A |
| HNRNPA1P8 | PEX11A | ATP5A1P3 | NEAT1_3 | ZDHHC20 | PSMA6 |
| SERPINB9P1 | NUP133 | STAT3 | CDC25B | ADH6 | GOLGA2P5 |
| RP5-971N18.3 | SKAP2 | RP11-886P16.10 | RP11-177H2.2 | AC007326.1 | LHB |
| CHMP2A | AC073342.12 | RP11-423G4.10 | CTD-3126B10.4 | RP11-20I23.11 | HNRNPA1P53 |
| TRA2B | MRPS18AP1 | MLYCD | CREB3 | CARM1 | NUFIP1 |
| ZNF823 | RP11-304L19.1 | RP11-261C10.5 | RP11-810M2.2 | ALMS1P | GTPBP8 |
| AC159540.1 | CTD-2021A8.2 | IL23R | MGC45922 | RP11-544I20.2 | LARP1P1 |
| TMEM14DP | ARHGEF28 | RBM34 | RP11-77H9.5 | HPR | GPX8 |
| SGK223 | CTD-2023M8.1 | AC006994.3 | RP11-359P18.1 | CFHR2 | PRR34 |
| RP11-376O6.2 | SUPT4H1P1 | RP11-33B1.3 | CTD-2325P2.4 | MRPS36P1 | PGBD5 |
| RP11-451F14.1 | THRSP | CBX1P1 | GATM-AS1 | AP006621.5 | KIF13A |
| CCDC68 | CDRT4 | FOXL1 | RP1-59D14.1 | TRAV36DV7 | PKNOX1 |
| RP4-678D15.1 | TUBB8P7 | TRIP13 | SPEF2 | GTF2H2C | MVB12B |
| CDO1 | CDC40 | ASH1L-IT1 | SETP17 | RP11-669N7.2 | RPSAP11 |
| RPS6P25 | FCF1P5 | GMPS | SEC24A | XXyac-YX155B6.7 | ZNF620 |
| KLF3-AS1 | XXbac- | SIK2 | PMEPA1 | RP13-228J13.8 | TMC7 |
| | BPG181M17.5 | | | | |
| NDUFS8 | NR0B2 | RP3-354N19.3 | C1DP5 | AC005329.7 | RPSAP72 |
| SSR4 | ZNF300 | RP5-1024G6.2 | RN7SL521P | B3GALT2 | RP11-254F7.4 |
| COL15A1 | WFDC3 | AP000560.3 | LY6G5B | KIAA1143 | SLC25A5 |
| RP11-848P1.7 | RP11-303E16.3 | RP5-1042K10.10 | AC007381.3 | RP11-4B14.3 | RP11-872J21.3 |
| AP2M1 | SPICE1 | GTF2IP14 | SGOL1-AS1 | RP11-149I2.4 | TRAF4 |
| KLK2 | RORA-AS1 | PDIK1L | SPTAN1 | RP11-284H18.1 | RP11-77G23.5 |
| PITPNA-AS1 | BCDIN3D-AS1 | LL0XNC01-36H8.1 | TRA2A | RP11-234O6.2 | RP11-52A20.2 |
| NSUN4 | PALMD | C2orf72 | IMPDH1P6 | UBE2V2P3 | FXYD5 |
| DHRSX-IT1 | DHRSX-IT1 | BTBD2 | ARPP19 | C9orf69 | PDCD6IPP2 |
| SLC5A9 | SMIM1 | RP11-546K22.3 | ANGPTL2 | RN7SL663P | RP11-629O1.2 |
| CIRH1A | RP11-91P24.5 | RP11-386M24.3 | VHL | FSCN1 | AC008746.10 |
| CLIC1P1 | NREP | DOCK4-AS1 | OAZ1 | ATP13A4-AS1 | FGL1 |
| LINC01134 | ZNF333 | CTD-2562J17.6 | RP11-144O23.22 | ZNF518A | RP11-63G10.3 |
| LINC00563 | MANEA | PGRMC2 | CICP20 | NPRL3 | ENTPD3 |
| CTD-2527I21.5 | AP000593.7 | CTD-2620I22.2 | AC000095.9 | RPL21P3 | ZNF830 |
| PRADC1 | ZNF567 | AURKA | KHSRPP1 | AL135745.1 | AC002400.1 |
| ZNF397 | TWF1P1 | PCDHB7 | SNRPA1 | AC005363.11 | MEIS1 |
| AC009120.5 | RP11-660M5.1 | CHMP7 | FAM187B2P | FXNP2 | ACOT8 |
| MYL6BP1 | ACOT12 | TANGO6 | INHBE | DUS1L | SOX5 |
| CFAP44-AS1 | UCP3 | AC016708.2 | SMAD4 | RP11-56B16.1 | LSM4 |
| ARRDC5 | EIF4H | MAFG | ALYREF | RP5-937E21.8 | CAMK2G |
| PTMAP1 | MS4A14 | AC012512.1 | C15orf52 | AC017081.1 | RIOK1 |
| RP11-797D24.3 | RP11-20G6.1 | PTP4A1P3 | FAM181A | PATL1 | RNU2-6P |
| ASPDH | WDR82 | ARGLU1 | PRSS2 | RPL37P2 | RGPD8 |
| PTPRE | MAP3K7 | SCOC | THPO | RPL3P2 | TMEM243 |
| CTD-2224J9.8 | CTD-2553L13.9 | TULP4 | RBM14-RBM4 | ZNF45 | AC004538.3 |
| RP11-455B3.1 | HCFC1R1 | TMED10P1 | HRAS | FTH1P20 | SHMT1P1 |
| RPL7AP15 | CCHCR1 | RP11-589P10.7 | RP11-330H6.5 | RP11-162A12.3 | C11orf73 |
| LTV1 | SELP | RP11-162G10.5 | CLDN1 | TMEM110-MUSTN1 | RP3-340B19.2 |
| AC005954.4 | TBC1D20 | RN7SKP23 | MYO15A | RP11-234A1.1 | PDIA5 |
| HEATR4 | AC141586.5 | RP11-388M20.1 | MEF2C-AS1 | PMPCB | MDM2 |
| RP11-834C11.10 | RP11-498M15.1 | NOL8 | NRAS | ITIH1 | SLC2A3 |
| RP11-384L8.1 | RP11-423H2.1 | KB-1732A1.1 | ISLR2 | DDX18P1 | RAD1P1 |
| RP11-175P19.2 | GABARAPL2 | VN1R1 | CPSF3 | MRPL27 | PGBD3 |
| KNOP1P2 | CISH | COX5B | FBXO8 | LINC00261 | MICAL3 |
| RP11-34F13.3 | CLTCL1 | TMEM241 | PPAT | USP22 | USB1 |
| FAM86EP | RFXAP | ZNF107 | EPRS | PDS5A | TRABD |
| KRT18P15 | GLE1 | OSBPL7 | RPL23AP20 | CAB39L | RP11-203B9.4 |
| TTC12 | CTD-2008P7.8 | ENDOU | RP11-440L14.1 | SNORD3C | RSBN1L |
| TRAPPC6B | ZNF695 | SRSF10P1 | CBL | RPL21P7 | DDX19B |
| C6orf99 | FGFR3 | MOCS3 | AKR1C2 | RP11-348N5.9 | RP11-89C3.3 |
| GOLGB1 | CPNE9 | LSAMP | NOP10 | RP11-570G20.1 | RP11-327F22.2 |
| GRIN3B | BHMT2 | MIR4432HG | PRRC1 | MAT2A | GPR3 |
| RP11-344B5.2 | SPTLC1P1 | RP11-1148L6.5 | NAP1L1P1 | TAC4 | HSF5 |
| ASB6 | SPDYE15P | SLC29A3 | LIPC | NPY6R | IGKV2OR2-7 |
| IGKV2OR2-7D | TEX14 | CFAP58-AS1 | GMCL1 | CTD-2308B18.1 | LRBA |
| MMAA | RP11-51F16.8 | CD200R1 | AL021807.1 | RP11-618P17.4 | CYTH2 |
| AGGF1 | ANOS2P | ZBTB3 | NAT6 | CD83 | UQCC3 |
| CRYZL1 | RP11-435I10.4 | MT1P1 | SLC43A2 | LTK | AF146191.4 |
| PXDNL | AP000266.7 | POLR2H | CTD-3149D2.4 | FAM114A2 | RP11-138B4.1 |
| ZNF628 | ERGIC3 | FAM184B | DNAJC27-AS1 | RP11-685M7.3 | VSX2 |
| PSMC1P5 | LDLRAD4-AS1 | PAK6 | RP3-370M22.8 | CTB-134H23.2 | UQCRH |
| POM121L8P | MYO5BP2 | RP11-875O11.2 | ITIH5 | XXbac- | FASTKD5 |
| | | | | B444P24.10 | |
| RP1-149C7.1 | OR5BA1P | CH17-270A2.2 | RP11-38J22.1 | RP11-380I10.2 | ATP5C1P1 |
| RPL5P22 | HNRNPA1P44 | HOTAIRM1_1 | AC011284.3 | C11orf74 | RP11-624L4.1 |
| Metazoa_SRP | RP11-731C17.1 | CTC-505O3.3 | ALKBH2 | HIST1H1A | SEPHS1P6 |
| PCSK6-AS1 | PRRT3-AS1 | RP11-173P15.7 | RP11-77O7.1 | CH17-478G19.2 | RP11-799M12.2 |
| CTC-534B23.1 | SPG21 | AC068522.4 | RPL21P120 | IGKV2-26 | PSMD10P3 |
| DNM1P38 | IGHV1-3 | RP11-573G6.8 | RP11-881M11.1 | RP11-696F12.1 | RP11-91H12.1 |
| CTD-2561J22.5 | RP11-674E16.1 | RP11-1057B6.1 | TSPAN5 | RP1-93H18.6 | BZW1 |
| RP11-767N6.7 | ARFGAP2 | CALR | ABCG5 | XPO1 | PRKAB1 |
| MRPS31P2 | C4B | EXOSC5 | AC079922.3 | CTD-2589H19.6 | ZFYVE19 |
| RRM2P3 | ZNF200 | LSM10 | MFAP1P1 | CTC-1337H24.3 | EIF4A1P5 |
| MGAT4B | RP11-498P14.3 | SLC6A8 | RP11-552M6.1 | ZNF483 | VAMP7 |
| VAMP7 | PRMT5 | IGFLR1 | TMEM94 | ACTG1P4 | CDK5RAP1 |
| RP11-257O5.2 | AKAP8 | MIEF1 | AC006946.12 | RP5-1174N9.2 | RP11-264M12.2 |
| PLA2G4E | TTLL8 | HK2 | TERF1P5 | RP11-166O4.4 | RP11-426C22.6 |
| CYB5R3 | CCDC69 | KANSL3 | EPHA10 | CHRNA1 | ZNF79 |
| CTB-131K11.1 | CTD-2228K2.1 | TTC7B | RP11-626G11.3 | TAF1A-AS1 | HNRNPA3 |
| RP11-96O20.4 | S100P | RP11-432F4.2 | CTD-3088G3.8 | FAM216A | RP11-10H3.1 |
| SDHAP1 | GCHFR | ARHGEF17 | MIR22HG | SRSF9P1 | PNMT |
| KIF21A | RP1-149A16.3 | CCDC84 | RN7SKP175 | NAA15 | RP11-179H18.5 |
| SGK2 | DPPA4 | CTD-2583A14.8 | COA1 | PACSIN2 | RP11-250B2.5 |
| CTD-2542L18.1 | ZNF2 | RP11-778D9.12 | KLHL8 | TIPIN | IGHG4 |
| POU5F2 | LINC00891 | RBM45 | RP11-395L14.18 | CTC-1337H24.4 | MPHOSPH10 |
| SCN11A | LYZ | RP11-1070N10.7 | KB-1440D3.14 | LIPT1 | ARMCX3 |
| RP11-778D9.13 | ADCY9 | MLLT1 | ST13P13 | RP11-108O10.2 | RPS12P28 |
| GPR62 | RP11-715J22.6 | CTD-3001H11.2 | RP11-30P6.1 | MAGEA9B | KRT18P7 |
| RP11-701H24.5 | ACP1 | PAXBP1-AS1 | PALM | IBA57-AS1 | RP11-328P23.3 |
| TJP1 | KLRB1 | PTCH2 | KCNK4 | YIPF1 | RALBP1 |
| WNT4 | AKR1A1 | QRSL1 | CTD-2528L19.4 | RP5-1139I1.2 | TPD52L1 |
| POLR1E | CTB-58E17.3 | RP11-736K20.6 | LAP3 | SMC6 | SH3BP5L |
| AC009133.17 | RPL7 | ZNF674-AS1 | CCDC71 | ZYG11A | RP11-178D12.2 |
| SPATA2 | RP3-425P12.4 | LL22NC03-2H8.4 | CCDC34 | RP11-497G19.2 | GIMAP4 |
| TUNAR | P2RY1 | RUNX1 | RP11-1006G14.1 | RP11-171I2.1 | PRR14L |
| MRPL35P3 | RP11-30L15.6 | MTRNR2L4 | CPSF1 | CHCHD1 | RP11-649A18.3 |
| RP11-295G24.4 | RP11-165M1.1 | CEP170 | CYP4F11 | RP11-800A3.3 | DDX51 |
| AC013460.1 | RP11-626E13.1 | MYCT1 | RP11-297C4.2 | GGTLC2 | AC004449.6 |
| CCT8 | MTMR12 | GFRA3 | RP11-533F5.2 | MYOZ2 | GOT2P3 |
| RNF181 | SNORA84 | LINC00618 | ITGAM | RPS14 | AC000095.11 |
| COPS7B | DNM2 | AC009299.2 | ATP13A2 | DNAJC3-AS1 | AC022405.1 |
| AC010412.1 | DBIL5P | RN7SL650P | GPAM | SF3A3P2 | C1orf127 |
| RP11-905K4.1 | KIF17 | AC006272.1 | CABP1 | AP000892.4 | SLC12A6 |
| TCEA1P4 | RP11-386G11.5 | RPL13 | TMEM203 | OR2W6P | FNDC1 |
| FAM35CP | Metazoa_SRP | POLE3 | MAN1C1 | RP5-965G21.6 | RIMKLA |
| RP11-383B4.4 | CPOX | CHST14 | RP11-728G15.1 | RP11-129G17.2 | FAM76A |
| RP4-740C4.7 | RP11-34E5.4 | CTC-344H19.4 | CCDC93 | RP11-44F14.5 | CACNB4 |
| RUVBL1 | LINC01535 | RP11-512F24.1 | USP37 | FXN | GLRA1 |
| E4F1 | TNFRSF11A | PSORS1C1 | RPSAP69 | ZDHHC9 | ANKRD30BP2 |
| RP11-122M14.1 | RP11-602M11.4 | GTF3C2 | RPS21 | SPEF1 | RPL7L1P8 |
| RPL9 | ENPP4 | CTD-2026K11.5 | IGHV3-73 | RASL11B | PSMD10P2 |
| KIAA1191 | TNS2 | METTL5 | RP11-325E14.5 | SEC11C | RP13-258O15.1 |
| RP11-378A13.2 | ZBTB12P1 | CTD-2619J13.3 | WSB1 | VAPA | LYSMD4 |
| APOA1 | NUDT21 | VPS36 | BTBD19 | PFN1P1 | ILF3 |
| RP11-364L4.1 | ANAPC2 | ELOVL2 | RP11-490N5.3 | TMEM9B | FAM3C2 |
| WEE1 | RP11-54O15.3 | AF129075.5 | DRAIC | RP11-400F19.12 | JUNB |
| RP11-573M3.2 | ORC3 | RBM7 | GK5 | GDPD2 | STK17A |
| RP11-146N23.1 | MGAT5B | RP11-115N12.1 | AC006014.7 | TRIAP1 | FERMT3 |
| PTPRN | GJA1P1 | IAH1 | CCT6P1 | PSMB7 | CALM3 |
| C19orf73 | TM4SF19 | NUTM2E | VWA3B | RPL29P12 | RP11-11C20.3 |
| EPS15 | RP11-587D21.1 | TP53I3 | ERCC3 | RP11-49K24.8 | RP3-415N12.1 |
| LSM3 | FARSB | PHF14 | CCAR2 | ZYG11B | FAM200B |
| ZNF764 | LRRC17 | RP11-455J15.1 | RP5-915N17.11 | POP7 | RP11-374M1.2 |
| PKD1P6 | FAM172BP | ABCB7 | FLT3 | CDC37 | ZNRF1 |
| RP11-295G20.2 | AP000280.66 | GLIS2 | GEMIN4 | POLR2B | CTA-384D8.34 |
| DDX18 | C14orf159 | CTD-2619J13.23 | MYD88 | LA16c-385E7.1 | MLLT4-AS1 |
| RP11-298I3.4 | RP11-681L4.2 | RPL32P26 | BCAP29 | RP11-113K21.6 | RP1-89D4.1 |
| TDGP1 | MTND5P14 | PRELID3B | GEMIN5 | DIP2A-IT1 | DNAJB12 |
| IGHV3-71 | GAS8-AS1 | GNRH2 | SMIM8 | FTH1P1 | AIFM3 |
| CTAGE6 | OGFOD3 | RP11-119F19.4 | ZNF699 | PLEKHO2 | LMAN2 |
| RP3-414A15.12 | MASP2 | SLC8A2 | IGHV3-30 | Metazoa_SRP | OR2C1 |
| ATP8B5P | CTD-2035E11.4 | NDRG4 | CTD-2286N8.2 | FOXD1 | YBX2 |
| MRPS26 | OR7D2 | CLEC2B | PXN-AS1 | PDLIM7 | NRTN |
| BOLA2P2 | AGRN | DEPTOR | RP11-386I23.1 | LAMTOR5 | DNM1P35 |
| PAWR | STT3A | DAPP1 | DDN | FAM133CP | LINC01587 |
| FAM41C | AC011558.5 | CDKN2A | ITM2A | TMOD3 | AC092192.1 |
| SOCS2 | RBPMS2 | RP11-355F16.1 | PPEF1 | RP5-1021I20.2 | YWHAZP2 |
| RP11-1070A24.2 | RAC2 | RPS2P41 | FLJ26850 | RP11-1000B6.3 | RP11-615J4.4 |
| CFAP99 | MTND4P14 | RP11-680F20.12 | BCL2L10 | RPS26P6 | PARP4P2 |
| RINT1 | CTC-498J12.3 | ZNHIT2 | CBX3P4 | FAM213B | SEC61A2 |
| MKRN1 | NUP50 | LINC00612 | UBE2A | RP11-120K19.4 | RP11-264B17.5 |
| USP47 | RP11-617F23.2 | WNT16 | RP11-367G18.1 | RP11-81A22.4 | IER3IP1 |
| LVRN | C10orf71-AS1 | RP11-38L15.8 | BNIP3P17 | CH17-264L24.1 | ATP5D |
| DST | AC005251.3 | RP11-124N2.1 | MTX3 | PABPC1P4 | CMYA5 |
| RP11-608O8.2 | OR2A4 | RP11-446H18.5 | DDX55P1 | RP11-104H15.7 | RPL22 |
| RP11-131L12.4 | CTD-2015H6.3 | ZC3H7B | LSG1 | CPB2-AS1 | RP11-111A22.1 |
| RP11-1012A1.4 | GSTM4 | RPGRIP1 | DPF2 | RP11-383C5.5 | RP11-627K11.6 |
| RN7SL798P | CTD-3194G12.2 | CTD-2283N19.1 | C2orf61 | AC005943.6 | RPL23AP67 |
| PLAC8L1 | RBCK1 | RP11-454K7.3 | DPY19L2P1 | RPL7P49 | CTD-2515O10.5 |
| RP11-18B3.2 | STX19 | DSG2 | RP5-951N9.1 | RP5-963E22.6 | DGCR6L |
| RP11-365N19.2 | SLC25A6 | SLC25A6 | HIST1H1PS1 | TAS2R30 | RP11-895M11.3 |
| CXCL11 | LXN | RP11-15J22.8 | ZNF587 | REEP3 | SH2D4B |
| TUBB1 | HDDC2 | EHD4-AS1 | KCTD9P4 | PRC1 | RP11-290H9.2 |
| AMZ2P1 | FAM86B1 | IGLC4 | SPATA17 | C19orf66 | RP11-413M3.4 |
| RPL5P12 | RP11-340I6.6 | PWWP2B | RP11-707G14.8 | PLSCR2 | CISD3 |
| APOA1-AS | AC008985.1 | ESPN | HSP90B1 | RP11-58K22.5 | SNORA18 |
| RP11-170N16.3 | RPS7P11 | RP3-414A15.10 | RP11-314A20.5 | ZMAT5 | MFSD10 |
| CTD-3035K23.6 | TRIM50 | FRG1CP | RP11-726G23.10 | CTD-2013N17.1 | CELF6 |
| GALNT16 | TRAPPC11 | ITGA11 | LYPLAL1-AS1 | CTC-492K19.7 | SPRYD4 |
| PLIN1 | RP1-102E24.10 | RP11-16K12.2 | MTHFD1 | PPM1B | DESI2 |
| RP11-159F24.5 | RP11-154D6.1 | AC091633.3 | BX255923.3 | RP11-756H6.1 | RP11-23J18.1 |
| EPX | CERK | RTKN | RP11-637O19.3 | CTB-102L5.8 | CTD-2588C8.8 |
| PC | GTF3C4 | RP11-98D18.9 | SORD2P | RP11-446H18.1 | LPGAT1 |
| SNRPB | RP11-443B7.3 | RP11-507K13.6 | RNASE10 | KRT17P8 | CDC16 |
| RP13-228J13.1 | AC093616.4 | ARHGEF12 | NCL | SCG2 | RP11-302F12.10 |
| CTC-246B18.8 | AC006538.8 | TUBB7P | ARHGAP33 | SNX15 | AP3B2 |
| CTC-448F2.4 | SFN | KIRREL | FAU | KCNH3 | RP5-864K19.6 |
| RP5-892K4.1 | RP11-777B9.1 | PFN1P4 | Metazoa_SRP | RP11-326L17.1 | RP11-568J23.1 |
| HLA-DQA2 | BCL2L12P1 | ZNF92P3 | RP13-88F20.1 | RP11-311D14.1 | CASP6 |
| RP11-563N6.6 | RP11-152H18.4 | AP006621.6 | FOXD4L4 | RP11-861E21.3 | MIXL1 |
| CHMP2B | RP3-461P17.10 | AC004951.6 | CTD-2199O4.6 | DIO1 | LINC00571 |
| PDZD7 | RAB38 | RP11-517P14.7 | MIEN1 | CTD-2616J11.2 | RFPL4A |
| OR1J1 | OFD1P17 | CIITA | RP11-64K12.8 | OR2B6 | SAAL1 |
| HAAO | PAAF1 | FAM86B2 | TDGF1P5 | RP11-549B18.1 | REEP6 |
| AC008982.2 | RP11-114H24.6 | FUT11 | PPM1G | RP11-430H10.1 | RNF215 |
| PLCB3 | GRAP2 | RP11-671E7.1 | FER1L4 | FKBPL | RP11-793H13.11 |
| CTB-35F21.3 | OR2A7 | ZCCHC12 | SNX4 | LL09NC01-251B2.3 | TBC1D16 |
| RP11-83A24.2 | CALHM1 | MTCO3P12 | Z95114.6 | RP11-334J6.7 | EEF1A1P9 |
| UBE2FP3 | UBLCP1 | RPL7L1P9 | MARCKSL1P1 | RP11-379B18.5 | FAM103A2P |
| ZNF670 | MSRB2 | POTEA | AC006378.2 | TYSND1 | ZBTB7A |
| HNRNPAB | LARP1B | KANK2 | ATP8A2P2 | TTC21A | RP11-432I5.4 |
| RP11-144C15.2 | PRSS12 | RP11-546J1.1 | PPIAP3 | RPS8 | NELFE |
| RP5-1065J22.4 | VWA3A | SHANK3 | RP11-14N7.2 | HSD11B2 | THUMPD3 |
| RP11-384M20.1 | FAM101B | RP11-493P1.2 | MCRS1 | RYKP1 | NAB1 |
| RP11-631M6.2 | PRR11 | LINC01436 | FER1L6 | SNTB2 | YRDCP1 |
| TDG | HNRNPA1P45 | RN7SKP225 | RP11-3D4.2 | CH507-145C22.1 | GP5 |
| RP11-197P3.5 | CRYL1 | CNOT1 | SH3GL1 | TMEM230 | ZCCHC2 |
| WWTR1 | TSPAN1 | NFYC | MIER1 | RWDD4P2 | NBAS |
| CTD-3051D23.1 | SRSF4 | RP11-90P5.5 | FOXM1 | RP13-126P21.2 | C1orf229 |
| RP11-423H2.4 | RP11-672A2.6 | TRAPPC3 | RP11-876N24.5 | STK35 | RAB1C |
| PPP2R4 | C12orf50 | RP11-47G11.2 | RP4-539M6.20 | ZNF638 | CTC-459F4.6 |
| RP11-105C19.2 | RP11-356B19.11 | LINC00551 | PPIL1 | MT1F | UHRF2P1 |
| RPL19 | RP11-4B16.4 | RAB4B-EGLN2 | RP11-804H8.6 | CCDC116 | HSPA8P19 |
| KIF1BP | RP11-447L10.1 | AC004878.2 | RPL23AP55 | ZNF24 | RP11-146E13.4 |
| CCDC155 | ALG3P1 | ADPRHL2 | RP11-561O23.9 | HLA-C | OR6S1 |
| LINC00271 | TADA2A | ANTXRL | ATG12 | AP000662.9 | KIF24 |
| FCN2 | C7orf71 | CTD-2653D5.1 | RP11-159F24.6 | RP11-484D2.2 | TMEM248 |
| CRBN | RP11-97O12.5 | QRICH2 | MTHFD2L | SMCO3 | EVPLL |
| POLG | LINC00987 | AC110926.4 | RNF5P1 | CCT4 | RP1-99E18.2 |
| TAOK1 | C19orf47 | CTA-384D8.33 | SACM1L | RPL14 | RP11-713M15.1 |
| ZFC3H1 | IGKV2D-28 | PFDN5 | PLEKHD1 | RBMXP4 | RPS15AP36 |
| FAF2 | S100A11 | FABP3 | ZNF25 | CMC1 | ACSL5 |
| CALML6 | SLC22A23 | AK8 | RP5-1107A17.3 | RBL2 | DYNLRB2 |
| GNB2L1 | RP11-1399P15.1 | DHFR | CBARP | ULK2 | EIF3B |
| RP3-476K8.3 | CHPF2 | CTB-129P6.7 | AC008074.1 | CTD-3074O7.5 | ULBP1 |
| SSNA1 | AL022476.2 | BCL2L15 | RP11-502I4.3 | AC093249.1 | HCG14 |
| RP5-874C20.8 | RP11-16L9.2 | ERAS | ZNHIT3 | RN7SL737P | RP11-493E3.1 |
| RP11-478C6.2 | FAM129B | KCTD2 | ZNF890P | AC104024.2 | MCM7 |
| C1orf101 | KRT18P57 | Z69666.2 | OSBP | RP11-361M10.3 | ZBED3-AS1 |
| IGKV2-28 | HLA-DRB1 | RP11-182N22.9 | HEATR1 | RP5-1007H16.1 | RAB40AL |
| AC004985.12 | EIF2B3 | DNLZ | BRPF3 | RPL7P57 | GPRC5B |
| RP11-91P24.1 | KB-1507C5.3 | SUSD2 | CATIP-AS1 | LINC01184 | C9orf173 |
| ALG1L2 | CTD-2616J11.14 | SDHAF4 | RP11-1114A5.4 | RP11-434P11.1 | TM6SF1 |
| GPS2P1 | RP11-134K13.2 | Metazoa_SRP | BNIP3P10 | RSF1 | SRP14 |
| CYTH3 | AC012442.6 | LINC01399 | CTA-722E9.1 | AC011406.2 | LUC7L3 |
| KAT8 | AF131215.2 | CTD-2574D22.6 | ARGFXP2 | ZNF880 | KCNQ5-AS1 |
| FAM177A1 | CCDC43 | POMZP3 | FBXO45 | RP11-198M15.1 | RPS15AP24 |
| LANCL2 | NFKBIE | FAM167B | RP11-613F22.8 | KLHDC8A | LRFN4 |
| RP11-80H5.2 | RPL27A | RP11-110I1.5 | LNP1 | RP11-18C24.8 | PHKA1P1 |
| DIEXF | TMEM63A | RP11-1E4.1 | PDCD2 | CTC-273B12.5 | RYR2 |
| CPEB2 | EFR3B | SETSIP | CTB-31O20.3 | HIP1R | USP41 |
| RPL34P22 | MAU2 | PDCD6IP | PNMA3 | RP5-1153D9.5 | RP11-24N18.1 |
| RP11-92F20.1 | EI24 | ZNF736 | RP11-51F16.1 | PNPLA8 | MTX2 |
| PITX3 | RP11-268P4.6 | RGAG4 | PHBP15 | HSF1 | RP11-118M9.3 |
| AC115115.3 | NRBF2P3 | KRT8P34 | RPS12P26 | PPIHP1 | PKD2L2 |
| RP11-181E10.3 | LY6K | RP11-395L14.17 | CTD-2203K17.1 | RP11-707P17.2 | RP11-749I16.3 |
| IGFBP7 | ATP10A | RP11-65L3.4 | MAP4 | RP11-550I24.3 | RP11-15B17.4 |
| RP11-248G5.9 | CXADRP3 | AC005757.7 | COX20 | NDUFC2-KCTD14 | RPL23AP74 |
| AC109642.1 | SLC22A14 | RP11-417L19.4 | NBEAL1 | ZNF575 | CTD-2302E22.5 |
| SMARCB1 | TRGV4 | KNOP1P4 | AC124944.3 | APP | MGP |
| RP11-6F2.5 | NUDT9 | HSPA12A | CTD-2373N4.3 | TUBGCP3 | CALHM3 |
| RP11-75A9.3 | ADGRE1 | SERF2 | RBM15 | RP11-255B23.1 | TAS2R42 |
| RP11-642A1.1 | ZNF282 | RP11-666A8.7 | L29074.3 | SUPT4H1 | ZFHX4 |
| XPOT | TUBGCP5 | RP11-142I2.1 | PYGM | ALOX12P2 | PTGES |
| RP11-80I15.4 | FAM92B | VN2R17P | CUBN | CBX3P1 | RP11-347C12.10 |
| CNN2P4 | ABHD11-AS1 | DDX53 | ASIC4 | OCIAD1-AS1 | NRADDP |
| RP11-320A16.1 | CCDC150 | NELFB | RP11-438J1.1 | RP11-7I15.3 | CTD-2260A17.3 |
| HMGN1P24 | TMC2 | LINC00266-3 | AC006547.15 | AL772307.1 | GFPT1 |
| RP11-472G21.2 | C21orf140 | LDHAL6FP | PARP8 | RBM27 | NCOA6 |
| GFI1B | RP11-401L13.7 | ZNF394 | U47924.31 | AL590762.7 | TNNC2 |
| DOT1L | PCED1B-AS1 | RP11-1084A12.1 | ZW10 | PARP4 | RPL23P2 |
| RP5-1198O20.4 | R3HDM4 | BAP1 | PPP1R16B | RP11-845C23.3 | ABHD1 |
| SCAMP1-AS1 | PROC | RP4-753M9.1 | CTD-2529P6.3 | RP11-147G7.3 | RP11-679C8.2 |
| THTPA | AQP6 | AC009303.3 | ICAM3 | OSTCP1 | RP11-34F13.2 |
| PART1 | ELOVL4 | NOSIP | RIMBP3 | LOX | AC109631.1 |
| C1GALT1P1 | RPL5P9 | NQO1 | TMEM171 | RP11-442G21.2 | ABL1 |
| ZFP36L2 | UBE2L4 | SEC1P | PPHLN1 | RP11-754B17.1 | RP11-82K18.2 |
| AC079779.4 | CDHR5 | TRIM71 | RP11-720D4.2 | TIGD6 | ZNF414 |
| AC129492.1 | PNP | SLU7 | FGFR1OP | AC009274.6 | LRRC31 |
| DBIL5P2 | API5 | AF111168.2 | CMTM2 | YDJC | C22orf29 |
| TAF7L | CSTF3 | WISP3 | AC062017.1 | OCLN | AC064836.3 |
| C11orf31 | GPBP1 | PCDHGA5 | RRAGD | RBM23 | ADGRV1 |
| TRG-AS1 | RP1-197B17.5 | RP11-1072C15.1 | RP11-79D8.2 | CAMK2N2 | MYOT |
| CTD-2587H19.2 | HK2P1 | CRYAB | RPH3AL | XKR9 | RNF26 |
| FAM163A | RP11-159N11.4 | IL1B | NUP160 | CNOT11 | RP11-96O20.5 |
| FAM83A | E2F8 | RP11-395I14.2 | RP5-973N23.4 | IPPK | RP11-761I4.5 |
| SIGLEC17P | RASGRP4 | SLC39A5 | BX004987.4 | FOXN3-AS2 | PERP |
| JMJD1C-AS1 | KAT5 | RP4-584D14.7 | KIAA1958 | ZNF484 | CRAMP1 |
| LRP5 | GGPS1 | RP11-265N6.1 | ABT1 | RPL35P1 | LL22NC03-30E12.13 |
| RP11-235C23.5 | CTD-2547L24.4 | FADS3 | OSBPL1A | AC007365.1 | IGLC5 |
| RPS10 | DPCR1 | CDIPT | LINC00942 | RP11-276H1.3 | LA16c-380H5.1 |
| WBSCR22 | CYR61 | RP1-130H16.18 | SLC1A1 | PDE12 | EIF2S2P3 |
| CATIP | HSPD1P6 | COL6A4P2 | UBE2Q1-AS1 | ACAT1 | PTP4A1 |
| RP11-167N4.2 | RPE | LTB | AC007193.9 | AC018755.16 | ECT2L |
| RP11-329J18.4 | EAF1 | UNC13D | NARF-IT1 | UTP20 | SSX1 |
| RP11-474G23.3 | TBP | MYO19 | EEF1DP1 | TGIF2 | RP11-196I18.3 |
| RANBP3L | CYP46A1 | SLC6A6 | KLHL2 | GAPDHP22 | CTD-319515.3 |
| NSUN5P2 | FBL | RP3-355L5.4 | CPEB1 | RP11-635N19.2 | NCBP2 |
| SLC1A7 | RP11-757G1.5 | RP11-85G20.1 | RP11-38L15.2 | SERBP1P5 | TAS2R6P |
| RLIMP1 | RP11-609L3.2 | PRDX1 | FOXO1 | HCG4P5 | MTCH1 |
| BNIP3P26 | VAC14 | RP11-514P8.7 | SEC22B | RP11-397E7.4 | RSBN1 |
| UNK | GORASP2 | CTD-2611O12.6 | RSF1-IT1 | RP11-77K12.3 | APOBEC3D |
| SVEP1 | DHRS4-AS1 | PCDHB19P | C10orf10 | RP11-195F19.9 | RP11-166B2.8 |
| ST7-AS1 | RPL23AP81 | RP11-756P10.6 | CDK11A | ARHGAP39 | GPATCH3 |
| C6orf183 | CTC-459F4.9 | ANKRD20A19P | ELP4 | H2AFX | FCF1P7 |
| DDTP1 | ZNF816-ZNF321P | RP11-38G5.4 | SYCE1 | EMD | RP11-108L7.15 |
| RP11-480N24.4 | RP11-503N18.5 | RAD50 | RP11-109A6.2 | RP11-497H16.7 | KLC3 |
| MAB21L2 | OR6E1P | PAICSP4 | GOLGA6L4 | RP11-256P1.1 | HIST1H3B |
| RCBTB2 | RP11-531A24.7 | ERI3 | CBR3-AS1 | KCND3 | RP4-673M15.1 |
| CTC-548K16.6 | CTB-191K22.6 | RP11-378J18.6 | RP11-235E17.6 | RPS20P35 | ISM2 |
| C7orf61 | EIF6 | PGK1P2 | RPL17 | STAP2 | LACC1 |
| RP11-44M6.3 | RP13-735L24.1 | FAM53B-AS1 | CTB-58E17.5 | UPB1 | ZGLP1 |
| TCF3P1 | RP11-480I12.9 | PABPN1 | SLC23A1 | RPL5P4 | RP1-224A6.8 |
| G2E3 | GRK6P1 | RP11-181K12.1 | LINC00240 | RAB40C | CYP20A1 |
| PTGDR2 | XRCC4 | PTGER3 | HMGXB3 | GSTM1 | RP11-3K16.1 |
| NANP | FAM86FP | RP11-613C6.2 | RP3-404F18.5 | HMCN1 | RP11-49C9.2 |
| RP11-423C15.3 | MARCKS | VIM-AS1 | ANKRD54 | ABI1 | MRPS23 |
| RP11-256I23.1 | FAM171B | RP13-15M17.1 | TAT | HHLA2 | PIGHP1 |
| RPL21P65 | HNRNPA1P14 | PWAR5 | RP11-190C22.8 | NTHL1 | CD99 |
| CD99 | WDR37 | ABCB8 | PBX2 | RP11-1096G20.5 | XX-C283C717.1 |
| PCOLCE-AS1 | ATP6V1A | GS1-257G1.1 | CTD-2545M3.2 | CNP | RP11-1250I15.3 |
| RP11-84A14.5 | SERINC3 | KLF2P2 | RP11-550A5.2 | FAM192A | HLA-L |
| MOB4 | TRIB3 | BEND3P3 | MAPK14 | RPL21P40 | RP11-76P2.4 |
| LINC00336 | RP11-154H23.3 | OTUD6B | RAPGEFL1 | S100PBP | PCDH11Y |
| SPATA41 | SCG3 | LSM2 | WASH1 | ACTL8 | FEM1C |
| RP11-485G7.6 | NACAD | B3GAT3 | RP11-497H16.5 | RP11-705C15.2 | PAH |
| AC124997.1 | SRPK1 | RPL32P29 | RP4-614O4.11 | CTD-2017C7.2 | TTC9 |
| RP11-416N13.1 | NOP58 | ODF2L | C6orf201 | CHMP4A | OR7M1P |
| TAP2 | DDX23 | RP11-34P13.15 | ATXN2 | TAS2R13 | RP11-327F22.1 |
| RP4-669H2.1 | PHTF2 | UBXN1 | RP2 | C6orf58 | NMRAL1 |
| RP11-19G24.1 | FAM13A-AS1 | RP4-625H18.2 | PPP1R1A | RP11-138I18.1 | HOXD12 |
| IMMP1LP2 | APIP | PAGR1 | SUDS3P1 | MADD | PRMT6 |
| CTB-50L17.9 | CCDC6 | ZNF675 | DUT | DENND5B-AS1 | RBBP6 |
| DIS3L2 | TM9SF1 | IGKV2D-29 | RP11-75C10.7 | CLCF1 | PMS2P3 |
| CCND3P1 | RPS13 | RPL7AP2 | PRPS1P2 | MYLK2 | GCNT1P1 |
| RP11-109P14.10 | AC006116.19 | PAK1IP1 | AC000089.3 | RP11-96D1.9 | RP11-182J1.5 |
| KRT8P29 | ESCO1 | CAPN7 | FAM91A3P | RP1-102E24.6 | STMN1P1 |
| LZTR1 | RP11-243M5.3 | FLJ12825 | RP11-114F3.5 | PPP3CA | MST1P2 |
| RP11-884K10.7 | APOC2 | LSM14A | ALG1L | CTC-508F8.1 | CALR3 |
| MTATP6P11 | PTDSS1 | ITGB6 | KRT18P55 | LINC00548 | PABPC4 |
| EEF1DP2 | GGA3 | PRPS2 | SPX | AL358852.1 | CTB-176F20.3 |
| ARF3 | SH3BGR | C11orf63 | PPP1R3G | COLQ | BTF3P6 |
| RP11-138A9.1 | RP11-512M8.5 | SETD8P1 | ZNF134 | AC010525.4 | LINC01132 |
| RP1-78B3.1 | EMBP1 | CD24 | APOBEC2 | RN7SL138P | RP11-764D10.2 |
| RP3-508I15.14 | NOL11 | TIMM23 | ACADL | SNX29P1 | MSH4 |
| RP11-549J18.1 | RPL17P50 | RP11-467D18.2 | RP11-848G14.2 | LINC00680 | HSD17B11 |
| RP11-485M7.1 | FRY | WNT3 | RP11-16O9.2 | RP11-55K13.1 | C21orf58 |
| WBP1LP2 | MBNL1 | PQBP1 | KRT8P11 | UBR7 | NDUFB7 |
| EPHA1-AS1 | RP11-380M21.1 | SEMA3G | NSRP1P1 | RP11-52L5.6 | AC010761.9 |
| RPL7P60 | AC241377.2 | RP11-1074O12.1 | AC015849.19 | HNRNPH1 | RP11-352G18.2 |
| RP11-700J17.2 | AF131216.1 | CTD-2373J6.1 | CASP8 | RP11-1221G12.2 | RAD1P1 |
| SDHDP6 | RP11-278C7.5 | WAC | CTD-2526A2.2 | ARIH2 | EXOC5P1 |
| AC018738.2 | PPIAP31 | RNF14 | SMIM7 | NICN1 | XXyac-YX65C7_A.2 |
| RABIF | EIF4G1 | DLL4 | HNRNPA1P16 | IDH2 | PHB |
| C18orf8 | P2RX5-TAX1BP3 | ERBB2IP | RP4-814D15.1 | RP11-668G10.2 | TSSK5P |
| FLJ27354 | RP11-530N7.2 | RP11-161H23.10 | SRGAP3 | ERICH6B | RAPGEF3 |
| WDR46 | SPDYE21P | RP11-796G6.1 | LILRA4 | CTD-3185P2.1 | RHBDL1 |
| USP2-AS1 | MICALL1 | RP11-173C1.1 | RP13-476E20.1 | EVA1B | OTUD4 |
| RP11-57H14.5 | BCL2A1 | RSL1D1 | SUZ12 | RP11-227D13.2 | NVL |
| KYNUP3 | GPR179 | EEF1A1P3 | ST20-MTHFS | FAM111B | RP1-168L15.5 |
| RP11-333O1.1 | ATP5J | RNMT | COL11A2 | ABHD18 | AC016907.3 |
| OSBPL2 | BAG6 | RP5-827C21.1 | TTC38 | CTD-2233K9.1 | POLR2J |
| GCNT6 | AC006509.4 | RSPH4A | LY86 | SNORA7A | C21orf62-AS1 |
| AC007952.6 | RP1-163M9.8 | RP11-744A16.4 | LAG3 | IQCH-AS1 | GTF2A2 |
| RP11-133N21.10 | TAF1D | MARC2 | RP1-13D10.2 | CTC-453G23.5 | PPP2R5D |
| GCOM1 | AC005614.3 | RP11-13N13.6 | ZNF430 | IMP3 | HNRNPA1P68 |
| MLH1 | GOLGA8J | SERPINH1P1 | IL17REL | RP11-360F5.1 | DMXL1 |
| SCN5A | NLRP1 | CXorf23 | KLLN | KCNJ10 | SP7 |
| PSORS1C2 | C1D | CTD-3096M3.1 | CASC10 | LZIC | PPAN |
| WDR61 | MT-ND4L | ANKEF1 | RP11-521C20.5 | ARL17A | FYCO1 |
| RP11-115H18.1 | FABP5 | DFNB31 | ZKSCAN5 | ADNP-AS1 | KLHDC9 |
| DNAI1 | NUDC | TRAK1 | PPP3R1 | ATP6V1G2-DDX39B | PSMB6 |
| RP11-374P20.4 | CTD-2265O21.3 | TAF15 | SRSF2 | RAB42 | RP11-1102P22.1 |
| RP11-20I23.5 | RP11-649A18.4 | MYLPF | SRSF8 | RP11-374M1.11 | ATF2 |
| AC195454.1 | IL13 | RBM28 | RNF125 | HEATR3 | MTRNR2L3 |
| DNAJC7 | C19orf48 | RP11-674I16.2 | SNX29P2 | RP11-267M23.4 | RP11-452I5.2 |
| STEAP3 | RP11-325E5.1 | LPAR6 | RP11-91I20.3 | KCNK15 | XPO4 |
| AP001350.4 | RPL18A | KARS | HMGB1P21 | ZNF887P | PSMC6 |
| RP11-800A18.3 | ZMYM3 | FAM157B | SEMA3B | DDX39B-AS1 | PIM1 |
| CFAP57 | RP13-554M15.2 | DHRS13 | PDPK1 | CES1 | CTB-33G10.1 |
| RPL5P30 | DNAJC19 | SLC33A1 | RP11-173A6.3 | ENPP7 | MAP4K4 |
| PEX5 | RP11-16E12.2 | AL023806.1 | KLHL1 | RP11-661A12.8 | CTD-2081C10.7 |
| PSMC4 | CD47 | AC016722.4 | NUBP1 | EXOC1 | RP11-17A1.3 |
| UPK1A | CTD-2020K17.4 | RASD1 | RP11-468E2.4 | RANGAP1 | VDAC1P11 |
| SNORA31 | ZBTB48 | MCM9 | RHOXF1-AS1 | GPR152 | RPS6P16 |
| RP11-46A10.5 | ARMCX7P | RP11-85K15.3 | DNAJB4 | JRK | RP11-141C7.3 |
| RP11-563D10.1 | CTD-2228K2.2 | SOBP | NAPG | RP11-109D20.2 | LINC01556 |
| GMFB | SRSF11 | NECAB3 | RP11-1024P17.1 | DNAJC25 | AP1G2 |
| LINC00960 | SPATA32 | HIRA | ZC3H10 | ACOT9 | RP11-452G18.2 |
| UBE2D3 | AP000892.6 | GSPT1 | CTD-2619J13.5 | RP11-323F24.3 | TNNI3K |
| RP5-1065J22.8 | RP11-244H3.1 | DUSP11 | DRAP1 | AC009095.4 | RP11-737O24.3 |
| OSBP2 | XXbac- | PTPN22 | ASH2LP1 | FBXO48 | CD79B |
| | B562F10.11 | | | | |
| NXT2 | ARL14 | GLIPR1 | UBE2CP2 | CCDC103 | KCNIP2-AS1 |
| ZNF398 | ZNF280A | ABHD17A | CHAC2 | INPP4B | AL133243.4 |
| VWA2 | HSD11B1L | RNF168 | RP11-15E18.1 | CTD-3065B20.3 | CHD9 |
| LINC00950 | DBNDD2 | RPS3 | CFLAR | TBL3 | ZDHHC5 |
| BRCC3P1 | PITRM1 | MYEOV2 | COA6 | API5P1 | ADAT1 |
| CTD-2132N18.3 | RP11-302B13.5 | CHN1 | MTSS1L | UCKL1 | RP4-806M20.4 |
| RP11-370A5.2 | GAD1 | GS1-124K5.4 | TAF11 | RPSAP36 | CPLX3 |
| RRM2P2 | MS4A1 | HIGD1A | SLITRK6 | RP11-212D19.4 | PRDX3P1 |
| CTD-3137H5.1 | E2F7 | CDH23 | RP11-327E2.5 | TAS2R19 | MPDU1 |
| KRT18P43 | TBX15 | SPTY2D1 | IPMK | RMRP | RP4-761J14.8 |
| S100A2 | DGCR12 | RPL7AP30 | CACNB1 | USP4 | RP11-445J14.1 |
| MAK | RP11-46H11.3 | FDX1L | BNIP3P4 | CTD-2325P2.3 | RP11-645C24.4 |
| RIC1 | C5AR2 | HAMP | MRPS24 | IGLVI-56 | BTG1P1 |
| FO538757.2 | AIMP1 | COPS5 | B3GAT2 | EMC4 | WDR45B |
| IL10RB | AC026150.6 | SIK1 | SAA4 | CTC-550B14.6 | OSGIN2 |
| MRPS11 | RP11-598P20.5 | EXOSC8 | CABIN1 | TSNAX-DISC1 | PGM5 |
| RP11-326C3.13 | RP11-195B17.1 | RP11-211N8.3 | UCKL1-AS1 | AC083843.4 | RP11-216L13.17 |
| STAT6 | ROCK1 | GNL3 | SPDYE12P | DFFA | TNP2 |
| AC026954.6 | VPS26A | RP11-66H6.4 | RP11-775C24.5 | SMIM17 | VPS4A |
| ZBTB32 | TXN | AP000436.4 | CTB-181H17.1 | JCHAIN | PLEKHH1 |
| RRN3 | ABHD14A | C5orf47 | RAD9A | AC068533.7 | HSPA4 |
| PPIP5K2 | TIMM10 | MSH3 | INF2 | DPP9 | CYB5D1 |
| POLR2J2 | DBF4 | FBXL15 | TEX30 | CCDC189 | CALM1 |
| CNBP | RP5-1071N3.1 | ATF6B | RP11-307C12.13 | NGLY1 | SRCIN1 |
| AOAH | RP11-756G20.1 | MARCH6 | RP11-415F23.4 | ADIPOR2 | KCTD11 |
| RPA2 | AC005618.8 | RP5-1180D12.1 | C2orf82 | HNRNPRP1 | RP11-439E19.9 |
| PIGX | ARHGAP1 | AC002398.11 | RAB5C | C7orf25 | MCTP1 |
| HHIPL2 | PPIH | ZNF280B | SQSTM1 | RP11-178L8.8 | RP11-77P16.4 |
| RP11-64K7.1 | KLHL29 | CH507-145C22.3 | AC002519.8 | BAG5 | CCDC13-AS1 |
| RP11-252A24.3 | TAS2R5 | HIAT1 | AMIGO2 | RP11-1379J22.5 | SMKR1 |
| VBP1 | OLA1P1 | GPBAR1 | TOMM20L | ENO1-IT1 | ZIK1 |
| SNORA71B | SLC25A25-AS1 | SLAMF7 | GP6 | SIK3 | TANGO2 |
| TMEM217 | SLC25A12 | AC144831.3 | DALRD3 | PTPRB | ALAD |
| AANAT | VSTM4 | RP4-681N20.5 | RP11-411H5.1 | MRPL15 | CDCA5 |
| CD22 | RAB11FIP1 | ATF7IP | PSMB9 | RP11-301L8.2 | GTF2H2 |
| RPL7P1 | CTD-2349P21.12 | RP11-665C16.9 | THBS1 | NKG7 | PSMA2 |
| GVQW2 | RABGGTB | LINC00847 | PLAT | GIGYF2 | ATP13A4 |
| DNM1P47 | RAB34 | SH3PXD2B | ANKFY1 | CMTR2 | AC026202.3 |
| CYP2D6 | DPRXP3 | ARHGAP35 | NFXL1 | ATM | MUT |
| PYCR1 | PDE6C | RSPH10B | RP1-224A6.3 | NOL4L | FKBP14 |
| TCEB3 | MAD2L2 | CCDC115 | RP11-195C7.3 | ASS1P1 | IREB2 |
| C15orf57 | FABP6 | ULBP3 | MAP4K5 | MRPS16P3 | RP11-248C1.3 |
| RP5-854E16.2 | NAA30 | CTC-265F19.2 | ZC3H11B | P2RY10 | NPFF |
| RP11-1008C21.2 | VN1R42P | TACC1 | HCK | LA16c-313F4.1 | RP11-362K14.5 |
| CLCC1 | RAB35 | CCNL1 | CICP4 | TP53TG1_2 | NSDHL |
| RP11-68E19.1 | CTD-2555K7.2 | CH17-264B6.4 | PFKL | CTD-2249K22.1 | MANBAL |
| WDR81 | LINC00969 | CTD-2013N17.6 | DPF3 | CTSD | RP3-402G11.26 |
| FGFR1OP2 | FBXW12 | HFM1 | BMS1P2 | PFN1P6 | RP11-864J10.4 |
| CSE1L | ZNF222 | ARSK | SMIM18 | SMYD3 | LA16c-360H6.3 |
| HMGN1P8 | TRIM60P18 | HOXA-AS2 | SMC5 | NCOA3 | SIKE1 |
| SYNJ2BP | CTAGE9 | NAA20 | TTC19 | TNK2 | DPY19L2P2 |
| RP11-383C5.3 | TRIM13 | FH | BANF1 | IQCK | TMEM255B |
| IGLV3-21 | NPAS1 | PIP4K2B | NT5M | HYAL2 | RP11-27I1.6 |
| RBM25 | RP11-247I13.11 | PPME1 | RP11-473O4.5 | RP11-804A23.4 | LANCL3 |
| CENPM | CTD-2302E22.6 | CTD-2196E14.5 | PLRG1 | WDR77 | UQCRC1 |
| NDUFB2 | RAB22A | KIF9 | AC005077.14 | PPAN-P2RY11 | ZNF771 |
| ASUN | DDX3P1 | NOP9 | RP11-640N20.6 | GATC | MC4R |
| C6orf47 | TIMM8A | C1orf27 | CTD-2522E6.4 | TRIM52 | KTI12 |
| RBM5 | BRD8 | BOP1 | RNF114 | IGHV7-81 | AC007292.6 |
| FAN1 | C11orf54 | THA1P | SKIDA1 | DCUN1D2-AS | BOK-AS1 |
| MED28P3 | LBR | PGS1 | RP11-452L6.7 | SSBP1 | HSP90B2P |
| PRR5L | SURF6 | ELFN2 | PTPN14 | RP11-126O1.5 | PTGR1 |
| ARHGEF37 | POTEF | HACD3 | RP11-160E2.6 | RP5-1061H20.4 | PSD2 |
| MCF2L-AS1 | RP11-143I21.1 | UNC79 | RP11-111M22.3 | NR1I2 | MB |
| GART | PTPN11 | CTC-378H22.2 | NKRF | RNFT1P3 | GAS6-AS1 |
| RP11-210N13.1 | RP11-346D14.1 | CENPU | RP4-616B8.5 | AVIL | RP13-753N3.3 |
| ZBTB43 | MIF4GD | CTD-2014D20.1 | SLC25A32 | RP11-20I23.6 | HMGA1 |
| CTCFL | PAQR4 | IGHV3-33 | RP11-231L11.3 | RP11-214F16.8 | NSRP1 |
| EGR3 | NEK11 | CTB-131B5.2 | ZWILCH | ISY1 | RP11-686O6.1 |
| ADAMTS4 | RP11-423P10.2 | RP5-1112D6.4 | CCBL2 | LAMC3 | CHORDC1 |
| DECR2 | SH2D3A | CPTP | AP003385.2 | SGMS1 | HECTD1 |
| PPP2R1B | CCDC114 | TFAP2A | AP000580.1 | USP44 | TTC1 |
| CTD-202417.13 | RP11-609N14.4 | PIK3CB | RP11-151N17.1 | AVPI1 | RP11-445L13_B.3 |
| WDR12 | AC145110.1 | MMP24 | SLC2A5 | LINC01137 | RP11-573D15.8 |
| CHAC1 | UBP1 | ATP5B | RP11-171I2.4 | S1PR1 | APAF1 |
| METTL2B | C14orf178 | CALM2P3 | NGDN | MIR1539 | AC005306.3 |
| TRMT11 | TMEM186 | WDR87 | WDR83OS | Metazoa_SRP | ALDH1A1 |
| C8G | RP11-624A21.1 | ZBTB26 | STRC | RP11-354E23.5 | RAB15 |
| GSTA1 | LINC00998 | ZNF101 | EIF4EBP1 | RP11-486I11.2 | SLC30A4 |
| PSMD5-AS1 | RP11-996F15.2 | GEMIN8 | USP16 | MICU2 | SRCAP |
| CHRNA9 | PCF11 | YLPM1 | RFX7 | LRRC8D | INO80B |
| RP11-745O10.4 | WDYHV1 | ADCK2 | LCN12 | RP11-225N10.1 | RPL9P9 |
| SNORA74A | TBR1 | RP11-348H3.2 | RFC1 | MGARP | SNIP1 |
| WASH7P | LINC00945 | EFCC1 | FTH1P12 | SCARNA13 | RP11-815I9.4 |
| MMP24-AS1 | APOBEC3C | EIF5 | MED4 | VIPR1 | CLDND2 |
| GFRA2 | RP11-165F24.3 | NHSL1 | TLK1 | AC136352.6 | LINC00493 |
| CTC-559E9.1 | LINC00176 | IPO13 | TFPT | BNIP3P37 | ZNF235 |
| IGHV4-31 | NPIPB4 | OTUB1 | AC003075.4 | SMG1 | FAM217B |
| C10orf131 | PLEKHA4 | HAUS1 | GRK5 | MIS18BP1 | C5 |
| PCGF3 | RP11-43F13.1 | POT1-AS1 | CNIH2 | RFX1 | DDAH1 |
| ERCC6L | AC005740.6 | FGF9 | RP11-325K4.3 | LAD1 | CD27-AS1 |
| TUBA4A | TMEM219 | GS1-259H13.2 | CYCSP10 | ATP10D | BBS7 |
| RP11-314N13.3 | ZNF668 | MESDC2 | NACA3P | RP11-333E1.2 | RP11-255E6.5 |
| RAI1-AS1 | CTD-2555O16.1 | FAM136A | AFAP1L1 | CTD-2666L21.2 | FBXO3 |
| ZC3H6 | ARCN1 | PA2G4P2 | C2CD2L | ERN2 | RP11-458J1.1 |
| CH507-24F1.1 | RP11-179B2.2 | NIFK | BLOC1S6 | ACSM3 | P4HB |
| KIF27 | ATP6V1C1 | SEC61B | RP11-440I14.3 | AC068134.10 | RP11-10C24.1 |
| RP11-154H23.4 | C4orf48 | MRPL4 | SVIP | CDK5RAP3 | RP11-803B1.8 |
| RP11-511B23.1 | TMEM245 | ACBD4 | NDUFS3 | RP11-178L8.7 | CDK12 |
| YARS2 | AP000487.5 | ZNF426 | FAT2 | MED13 | MT-CYB |
| AP001059.6 | UBE3C | RP11-96H19.1 | DDX49 | ZFYVE28 | IL27RA |
| CORIN | PCDHB18P | DCK | A1BG | FTSJ3 | MTERF3 |
| SLC15A4 | PAX6 | RP11-680G24.4 | RP11-301G19.1 | RP11-749H17.1 | ACTR8 |
| RIMS4 | UQCR10 | SNX3 | STT3B | ZNF592 | CARS |
| TAF1L | RP4-592A1.2 | TIMM10B | ATP5J2 | ZCCHC8 | AHSG |
| RP11-799D4.4 | AK4P1 | KIF1B | DNAH7 | RP11-100G15.3 | C12orf43 |
| LSM6 | TRIP11 | COPS8 | PMS2CL | TAF2 | RP1-228P16.1 |
| ZC3HAV1 | PRG4 | DCAF4 | RP11-36I17.2 | PTP4A2 | RIC8A |
| GXYLT1P5 | TCP10L | SF3B3 | NRIP2 | CTC-479C5.12 | RP11-1212A22.4 |
| RP11-583F2.1 | WNK4 | SAMD1 | KRT7 | SNU13 | RPS3A |
| MDC1 | C16orf46 | NSUN3 | RP11-568J23.2 | CNIH1 | MTCO1P30 |
| ZNF614 | APLP1 | MTL5 | COG8 | RP11-296E7.1 | CCPG1 |
| COG7 | TOMM6 | RP11-16L9.1 | GIGYF1 | RP11-420A6.2 | NEU3 |
| HEBP1 | C12orf49 | RPS4XP1 | TUSC2 | RP11-319G6.1 | UTRN |
| AP001046.6 | SLC25A20 | FAM87B | VHLL | TMEM81 | ATF7IP2 |
| MPI | IQGAP1 | TGFB1I1 | PTGES3P4 | AP001042.1 | POLR2CP |
| GLTSCR2-AS1 | ALG1L9P | RP11-561C5.4 | SRGAP2C | RP11-571L19.8 | GPR180 |
| MYL12BP2 | AC098820.4 | RP11-539L10.3 | MST1 | RP11-1148L6.9 | PDF |
| ADPGK | ULK4 | CEP57 | SELT | ACAP2 | FSD2 |
| RP11-89C3.4 | LINC01426 | BNIP3 | FDXACB1 | LINC00662 | CTD-2267D19.1 |
| TGM1 | AC006042.8 | PSMA2P1 | RTCB | C9orf114 | RP11-456P18.2 |
| BCL7B | GUF1 | CCDC153 | ARPIN | SDR39U1 | RP11-448A19.1 |
| LL22NC03-63E9.3 | RP11-15K19.2 | TMSB4Y | LETM2 | CTA-228A9.4 | DQX1 |
| ZNF658B | ARPC1A | ANKRD18DP | UBE2J1 | SNAPC3 | IGKV5-2 |
| ESAM | CARNS1 | FAM199X | OLFML3 | RP11-359B12.2 | SERHL |
| DLK2 | HOXA11 | SMARCAL1 | XKR8 | LGALS4 | NOXA1 |
| RP11-338K13.1 | UPF3AP3 | BRE | ATP6V1G3 | MFN1 | PINK1 |
| ARHGAP9 | CTD-2206N4.4 | GPR160 | COQ10A | RTFDC1 | RP11-466J24.1 |
| RP11-106D4.2 | RP11-20D14.6 | DNAJC10 | RP11-158H5.2 | CFAP97 | PEX13 |
| RP5-1021I20.6 | MYLK | TPI1P1 | DNAJC1 | MRPL54 | BTN3A1 |
| TMEM220 | RP11-120M18.2 | NPC2 | RP4-561L24.3 | KLHDC10 | SNHG19 |
| ASB1 | RPS4XP2 | PFDN6 | RPL24 | RP1-111B22.3 | RAB1A |
| OIP5 | GRM1 | SERGEF | FASTKD3 | ZSCAN5A | MRRF |
| CHCHD3P3 | LINC01422 | DPY30 | LYRM9 | ATP5I | FUNDC1 |
| ATG7 | SRRM5 | COL25A1 | PSMB5 | PSMB2 | RP11-434D12.1 |
| EEF1A2 | RP5-1120P11.3 | LINC00309 | LINC01189 | USP9X | MYC |
| RP11-791G16.4 | NFIB | RP11-12M9.4 | CHM | NOB1 | RPSAP19 |
| PANK2 | FAM126A | RP11-271K11.1 | RP11-20G13.4 | SND1 | NOL6 |
| RP3-467N11.2 | PHF10 | RP1-281H8.3 | HSPA12B | RN7SL49P | RABL2A |
| OCIAD1 | RP11-15A1.4 | KDM4A-AS1 | LIN7A | PTPRCAP | MICALL2 |
| MTMR14 | BPHL | RP11-145M9.5 | LUC7L2 | AC000123.2 | PPATP1 |
| NFATC2IP | MFGE8 | PROZ | RP11-795F19.1 | ZGRF1 | RCOR1 |
| LIMK2 | MPHOSPH6 | MTND1P11 | FTH1P2 | GOLGA4 | WNT6 |
| DDX21 | CTC-559E9.13 | UBFD1 | SLC2A4 | AL136097.1 | KNG1 |
| TMEM180 | RP5-1157M23.2 | CLK1 | RP11-981P6.1 | COQ9 | POLQ |
| CARD8-AS1 | NUFIP2 | SNAPC5 | LL22NC03-80A10.11 | EPAS1 | ENO3 |
| ZNF564 | RP11-1148O4.1 | LONRF1 | RP11-111K18.2 | RP5-1159O4.2 | CTD-2331H12.7 |
| RSL24D1 | RP11-214K3.24 | RP11-20B24.7 | UBL7 | RP11-598P20.3 | SUMO2P19 |
| RP11-640N20.9 | INAFM1 | WFIKKN1 | FXYD7 | DAAM1 | TMEM167A |
| ACYP1 | GNS | ZNF420 | RBM26 | CTD-2293H3.2 | RP1-267D11.6 |
| NHLRC1 | SLC9C1 | CTC-542B22.2 | RP11-76C10.6 | RP1-224A6.9 | SEC13 |
| AC074183.3 | RP11-498C9.13 | MLLT10P1 | TRRAP | F12 | RP3-400B16.1 |
| RGS9BP | RTF1 | PTPRVP | FAM134C | RP11-475I24.8 | YES1P1 |
| PCDHGA2 | DYRK1A | LONP1 | RP11-723J4.3 | ZNF81 | FTH1P10 |
| TEFM | CTD-2049O4.1 | RP11-1C8.6 | TNIP2 | KRR1 | ABI3 |
| CTC-297N7.1 | RNF13 | STRAP | AP3M1 | ATP6AP1L | RP11-701H16.4 |
| PTPMT1 | RP5-991G20.2 | RP11-887P2.6 | LAMTOR3 | LINC01602 | C7orf43 |
| ALDH8A1 | MITD1 | SRR | USP3 | CDK11B | HLA-H |
| PRMT3 | ZNF664 | ICE2 | RP11-431M7.3 | TSSC1 | TEKT2 |
| RP11-231E19.1 | MAD2L1 | RP11-946P6.6 | TNFAIP8 | CAPN2 | LSM7 |
| T | RP11-17E13.2 | PALM2 | CSK | SPG7 | RP11-152F13.8 |
| HAO1 | CYCS | TBC1D3L | PPP2R2A | SKA3 | LLNLR-246C6.1 |
| RP11-379K17.9 | ZFHX2 | RP11-432B6.3 | BLMH | CTC-215O4.4 | C21orf91 |
| DCTN5 | LINS1 | UBIAD1 | GOSR1 | SRP72P2 | RP1-69M21.2 |
| SLC39A12 | RGP1 | FAM186A | TMTC3 | LA16c-313D11.12 | DNTTIP1 |
| GPCPD1 | DCP2 | CTB-50L17.14 | VPREB1 | AC002398.9 | NAV3 |
| APOO | RP11-806L2.2 | APH1B | PRPF38B | RP11-895M11.2 | CTD-2145A24.5 |
| AC074367.1 | NOL10 | SEC61A1 | ZMIZ1 | RN7SL81P | CXCR3 |
| RP11-1293J14.1 | AC016910.1 | IDH3B | ANKLE2 | AC005104.3 | CTD-2349P21.1 |
| DCDC1 | ISCU | CYP2D8P | SLC5A6 | PCMT1 | RP11-318E3.9 |
| RP5-1112D6.8 | RP11-61N20.3 | DCTN4 | LINC01260 | RP5-867C24.4 | ZCCHC10 |
| RP11-79P5.9 | RP11-45A12.1 | MTBP | TSPAN4 | AGR3 | FBXL18 |
| RP11-649E7.5 | RP11-151A6.6 | CD28 | SEPT7P6 | TSSK3 | SEMA4B |
| SIRT2 | TRMT10A | RPL7A | LATS1 | RANGRF | FRG1HP |
| RP11-180O5.2 | TNFRSF14 | RBBP5 | NDUFA7 | UFSP1 | AC093162.5 |
| CPT1B | RP11-325P15.2 | KBTBD7 | RP4-616B8.4 | KIAA1549 | SRSF10 |
| NOA1 | YWHAE | CTD-2152M20.2 | GDI2 | XXbac- | ATP5G1P4 |
| | | | | B476C20.13 | |
| PLGLB2 | RP11-486F17.1 | ST5 | MED10 | RP11-214J9.1 | CTD-2044J15.1 |
| MFAP5 | BOK | RP11-569G9.7 | RAD21-AS1 | LRRC27 | CCDC130 |
| OGFRL1 | AC006942.4 | EXOSC2 | RP11-379F4.9 | SLC35B3 | FLJ38576 |
| NACA | FASTKD2 | IGKV1D-13 | FGL2 | DYNLT3 | RECQL4 |
| SUGP2 | RLIM | SENP5 | RAB24 | RP11-414J4.2 | PIH1D1 |
| RP11-385D13.1 | VDAC1P9 | RP11-194N12.2 | SEPHS2 | RP11-342K6.4 | BAZ1A |
| LINC01534 | VDAC3 | EIF4E | RP11-700A24.1 | NUP54 | ICOSLG |
| RP11-46C24.7 | RP13-516M14.10 | RP11-559M23.1 | NECAP1 | ZNF100 | RP11-65D17.1 |
| PCDHGA7 | PLAC8 | TSEN34 | RP11-137H2.4 | GPR143 | GTF2E1 |
| RP11-266L9.5 | OXCT2 | NME3 | AC022154.7 | ORC2 | CDC45 |
| LRRC8B | DHRS3 | RP11-39K24.4 | FAM188A | ARVCF | SBF1 |
| UHRF1BP1 | RICTOR | IGHE | LINC01473 | FAM214A | ITFG2 |
| GBP1 | TRMT10C | AL133245.2 | KREMEN1 | FAM89B | CNKSR1 |
| RP11-245J9.5 | MTRF1L | NIM1K | FAM102A | AEN | C2CD4C |
| LINC00167 | MEMO1 | LINC01185 | COQ3 | PSME1 | GCLM |
| PROSER2 | CD44 | RUFY3 | POLE | CDCA4 | RP13-131K19.1 |
| CTA-989H11.1 | KLHL36 | TMEM75 | CMIP | FKBP3 | SLC39A2 |
| RRP9 | PLPP2 | SLC16A6 | RPL3P7 | RP11-264J4.8 | CCDC54 |
| HNRNPA1P70 | MIS18A-AS1 | AC008993.2 | SPTBN5 | ZNF732 | RPAIN |
| POU5F1P3 | CICP23 | HERPUD2 | PRKDC | RP11-164J13.1 | USP10 |
| FOXJ2 | TMEM45A | AC004549.6 | RP11-543B16.2 | ACTG1P14 | RHBDD3 |
| FANCF | TRAPPC2 | NAPSA | FAM8A1 | PPFIA4 | ISCA1 |
| RP3-337H4.6 | HMG20B | RP4-801G22.3 | GPBP1L1 | PRKACA | RP11-295P9.13 |
| RP11-250B2.4 | LINC01619 | HIST1H1E | RBM48 | RPL6 | RP11-363N22.3 |
| MYSM1 | RP11-421E14.2 | ARF4 | DTD2 | PTPRA | TMED5 |
| Metazoa_SRP | RAPSN | RP11-173A16.1 | FARSA | RP11-168O16.1 | RN7SL559P |
| ST3GAL1 | SF3A3P1 | TAPBP | VAPB | GALNT4 | ZNF713 |
| CYP51A1P1 | GTF2H5 | OR2AG2 | RP11-284M14.1 | RP11-102M11.2 | GPS1 |
| RP11-46H11.11 | VAV2 | MPPE1 | BRAP | RPS7 | RP11-449G16.1 |
| PVRL1 | ZNF681 | KIAA1109 | RP5-902P8.12 | CENPH | RP11-410L14.2 |
| BAK1 | SMAD9 | RP11-227G15.3 | ERICH6-AS1 | SORBS2 | RPL27 |
| RP11-181D18.4 | CANX | SDHB | IGLV11-55 | FAM208A | ZNF746 |
| CTD-2649C14.3 | RPL7AP65 | RP11-739N20.2 | FLVCR1-AS1 | RP11-519C12.1 | GFI1 |
| RP5-1165K10.2 | RPS20P4 | MOCOS | MRPS31P4 | ANKMY1 | MAF1 |
| DHRS7 | RSPH6A | ZBTB9 | DTHD1 | CA13 | RP11-295D4.4 |
| HEMGN | COPS6 | GLRX2 | MTMR8 | RP11-473M20.5 | NDUFV2 |
| FAM217A | NLRP14 | WDR7 | RP6-159A1.2 | CTC-366B18.2 | USP24 |
| TXNDC12 | HAUS6 | RP11-454F8.4 | LINC00528 | MRPL40P1 | ALKBH5 |
| PDHA1 | ATL2 | THOP1 | SNX1 | BACE1 | IBA57 |
| AFG3L1P | ASXL2 | ADPGK-AS1 | RP4-584D14.5 | RP11-390K5.6 | NBN |
| TMEM251 | RP11-421L21.3 | RP11-597A11.4 | RPUSD2 | PIGS | TMEM150B |
| ZNF558 | SAPCD2 | POM121L1P | ZNF396 | RIN2 | ZFPM2-AS1 |
| NDUFV2P1 | ZNF146 | ASAP2 | CTD-3154N5.2 | ODC1 | CTA-363E6.8 |
| MXD4 | EPB41 | ASF1B | SLC2A3P4 | RP11-755F10.1 | RNF126 |
| ALDH2 | SUPT7L | RP11-69J7.1 | RP11-386J22.3 | RP11-411B10.7 | SERPINE1 |
| RN7SL834P | BTN3A2 | RP4-564F22.5 | BNIP3P24 | CTC-325H20.7 | LRP2BP |
| PRKAR2A-AS1 | FAM3C | NCAPD3 | RP11-728K20.3 | TTLL5 | NEO1 |
| LENG8 | SSR3 | CDK5R2 | RP11-762H8.2 | PRKRIR | RP11-107E5.3 |
| MTR | GCFC2 | AC007292.3 | TREML2 | RP5-1021I20.4 | UBE4A |
| CORO1C | HENMT1 | RP3-449O17.1 | TOMM5 | RP11-290F5.2 | MED7 |
| IFT20 | CCDC82 | BTF3 | RP11-30K9.5 | PIAS2 | PIF1 |
| AC145124.2 | UBXN11 | RP11-140L24.4 | NR2E3 | C5orf45 | RP11-347C12.11 |
| ABCD1 | CYSRT1 | RP5-1021120.1 | RP11-537H15.3 | PMS2P6 | RP11-120E11.2 |
| DRICH1 | LA16c-60D12.1 | RP6-218J18.2 | WASF4P | RP11-307L14.1 | GPR150 |
| GJC1 | RP4-647C14.2 | AL590762.11 | RP11-727A23.7 | CTD-2210P15.3 | RNVU1-7 |
| GNPTAB | CH507-9B2.9 | AP000692.9 | YAF2 | NOP56P3 | ERGIC2 |
| VCPKMT | RP11-98J23.1 | UBOX5-AS1 | STXBP2 | RP11-488L18.4 | RP11-736N17.11 |
| MTND4P12 | RP11-169K16.8 | RP11-345J4.5 | C7orf57 | NFIC | SEPT14P19 |
| RPS28 | RP11-142L4.3 | ATP6AP2 | PAXBP1 | SCARNA10 | ATP6V0E1 |
| RASL11A | OSTF1 | LUC7L | UBE2L6 | ACAT2 | FN1 |
| HIVEP3 | HSPA1L | RP11-103B5.4 | RP13-514E23.1 | TMBIM4 | DDX58 |
| DHRS4L2 | CDR2 | TAF1B | NCR3LG1 | C17orf47 | RP11-295G24.5 |
| CNDP2 | PHKA2 | LSM1 | ESRRA | AHNAK2 | GOLGA8S |
| RNF219-AS1 | WNT2B | SMG1P3 | RP11-837J7.3 | RP5-1112D6.7 | RP11-667K14.5 |
| RPL21P122 | RP11-676J12.9 | BSPRY | HOTTIP | SPDYA | COPE |
| FAM186B | LINC01484 | BTF3L4P2 | BLOC1S3 | RP11-44F14.6 | AC005785.5 |
| ADAM29 | RP11-536C5.7 | SMYD4 | LLNLR-222A1.1 | COX7A2L | RP5-890E16.5 |
| AHCTF1 | DSN1 | ERO1B | RP11-477N3.1 | RP11-426D19.1 | NUP35 |
| RP11-681H18.2 | RP11-539G18.3 | PHF5A | RP11-331F9.4 | RP11-210K20.5 | IMMT |
| ALDOA | FRG1 | PAOX | THOC1 | RP11-705C15.4 | RP11-1020A11.1 |
| CTB-50L17.5 | AC010132.11 | DNAJB11 | GNAO1 | RPL11 | RP11-161H23.5 |
| TF | RP11-385F7.1 | HDAC1 | RP11-15E1.5 | PPIE | CTD-2301A4.3 |
| RP11-667K14.3 | RP11-165D7.5 | TMEM145 | TM4SF20 | BUD31 | CTD-2083E4.4 |
| HLA-K | CRLS1 | LINC01506 | RPS2P32 | RP11-416N2.4 | RP11-570P14.1 |
| CTD-2248H3.1 | GORASP1 | ACOXL | GAPDHP52 | ELMOD2 | RCOR3 |
| RP11-242J7.1 | C11orf98 | DOK4 | AURKB | RP11-475I24.3 | CYB5RL |
| TMEM14C | DUS2 | RP11-110I1.11 | RP11-803P9.1 | PCNPP1 | HDHD2 |
| SMYD5 | MIR194-2HG | ZNF70 | ZNF618 | HES1 | CEP131 |
| AC007038.7 | ARF1 | CCL5 | CTD-2561B21.8 | CHURC1-FNTB | LA16c-358B7.4 |
| RP11-707O23.1 | CALU | PIBF1 | AP000255.6 | RP11-474D14.2 | RP11-297D21.4 |
| PHACTR4 | RP11-982M15.8 | THAP5 | TLR6 | RERE | RP11-1046B16.3 |
| DET1 | SLC44A4 | RP11-316M21.7 | VCPIP1 | ZRANB2-AS2 | ECSIT |
| DDR1 | KLF2 | RBM43 | SZRD1 | CMAS | PMCH |
| DIXDC1 | CRYBB2P1 | HLA-DOB | PRPF4 | RP1-30M3.5 | CTC-366B18.4 |
| RP5-908M14.9 | RP11-212P7.3 | RP11-424M24.3 | RP3-425C14.5 | AC007389.1 | VPS26B |
| NAF1 | URI1 | CTC-260E6.4 | FNDC8 | RP11-493G17.4 | PRKRIP1 |
| CTD-2616J11.3 | SLC39A7 | SNHG21 | DPY19L3 | ACTG1P17 | MON2 |
| RAPGEF6 | EIF2B2 | RPL12P9 | GOLGA5 | BMS1P10 | RALY-AS1 |
| RP11-334J6.6 | CTD-2510F5.6 | NDOR1 | METTL3 | GS1-293C5.1 | FANCE |
| SUSD1 | HIST1H4E | IGLV3-9 | PHBP2 | EIF3F | CRHBP |
| HM13 | SNX5P1 | RP11-455F5.5 | BFAR | OASL | RP11-463I20.1 |
| RP11-159D12.8 | CASP4 | ESYT1 | DLEU1 | DCHS1 | EDRF1-AS1 |
| B4GALT6 | LRRC58 | ZDHHC11 | RPL30P11 | RP11-484D2.3 | HGSNAT |
| ALOX15 | TNPO1 | KIAA0196-AS1 | AC024937.4 | DDX56 | RP1-317E23.3 |
| GLG1 | FUBP3 | CTD-2313J17.5 | PLCH1 | TRMT13 | KLRD1 |
| ZNF689 | ATP5L | DHDDS | RPL17-C18orf32 | RP11-424C20.2 | ZRANB2 |
| SS18L1 | AC090505.6 | ZNF33A | RP11-467L13.7 | NHLRC2 | RP11-139H15.6 |
| NUTM2F | VNN2 | A2M | ZNF696 | TRAPPC5 | RP11-662B19.2 |
| RP11-410E4.1 | RPL7AP11 | RP11-676M6.1 | FAM21C | SYT1 | LMO2 |
| RP1-313I6.12 | ODF2-AS1 | INTS6P1 | FAM96AP2 | CENPB | CDC123 |
| HDHD3 | DNAH12 | EVPL | PPIAP29 | ADSS | CREB1 |
| GMPR2 | CTD-2013N24.2 | GPR135 | PROSER1 | OR1F1 | RPS16P9 |
| ZHX3 | RP11-45A17.4 | RP11-108K14.8 | RPL18P10 | HIST1H3E | C12orf73 |
| INO80E | BRD2-IT1 | TCAIM | RP11-57A19.5 | LINC01268 | MRPL34 |
| NOC4L | GTF2F2 | RP11-203H19.2 | FNTA | AC073063.10 | STX5 |
| MRGBP | METTL23 | RPS3AP25 | CMTR1 | IRF9 | SHQ1 |
| TMEM62 | DDX26B-AS1 | SASS6 | PPARGC1B | RAP1A | GTF3A |
| CYB5R1 | RP11-58A11.2 | PWWP2A | CTD-2017D11.1 | RAP1GAP | RP4-635A23.4 |
| PCDHGC4 | KCNC4 | TUBB8P8 | ZNF213-AS1 | DENND2D | DOCK9 |
| RP11-147L13.12 | PAN3 | CTD-2184C24.2 | EXTL1 | RP11-87E22.2 | TMEM9 |
| RHBDD2 | RP11-63K6.1 | RP11-264E20.2 | ARHGEF1 | YY1 | SUGT1P2 |
| GTF2H3 | RP11-817I4.2 | SLC30A5 | APEH | MIPEPP3 | TRIM74 |
| RP4-613B23.5 | RP11-894P9.1 | BNIP1 | CPED1 | ARFIP2 | CORO7-PAM16 |
| GPI | NOL3 | ABHD14B | RP11-545E17.3 | TEX261 | IKZF2 |
| COMMD5 | RN7SL75P | TMEM126B | RP4-616B8.6 | MPP5 | RP11-503P10.1 |
| PCDHGB8P | HLA-DMA | ATP2A1 | DCUN1D5 | SOCS7 | PSMB1 |
| PPP6R3 | CTA-268H5.12 | ITGB3BP | RP11-775C24.3 | HIST1H4D | TMEM189 |
| SEMA6C | TMEM88 | MPHOSPH9 | SYAP1 | FBXO41 | CTD-2036P10.6 |
| IQSEC1 | XPOTP1 | RP11-23P13.6 | C4orf47 | RP11-731F5.1 | RAB6A |
| C1orf131 | BIRC6 | CTC-251D13.1 | INAFM2 | CTD-2396E7.10 | AC005253.2 |
| NOL7 | SEPT7P7 | ATP6V0B | CHRNB1 | TMEM200B | EXOC3 |
| HCN3 | ZDHHC3 | HAUS7 | PLIN5 | LRGUK | SUSD6 |
| NEMP1 | USP45 | RP3-455J7.3 | ETFA | RP11-158L12.4 | COA7 |
| TIMP1 | KLRC4 | PTOV1-AS1 | DSTNP1 | SMCR8 | ACTG1P10 |
| RP11-572B2.1 | CTD-2002H8.2 | ZNF763 | CHPT1 | RP11-407G23.7 | RP11-268J15.5 |
| RP5-994D16.3 | RP11-136K14.3 | LA16c-325D7.2 | RAB30-AS1 | MAP3K2 | ECE2 |
| CH17-80A12.1 | RP1-117O3.2 | USP43 | RP11-54C4.3 | VTI1A | WASH2P |
| NCAPG2 | HSPA6 | SVBP | CHI3L2 | RP11-229D13.3 | AC106875.1 |
| CBWD2 | CTD-2086O20.1 | RBBP4P2 | MRPL41 | NEK4 | DOC2GP |
| SEMA7A | NAPA-AS1 | SFT2D1 | RP11-142E9.1 | SF3A2 | DTX3L |
| AC010761.13 | GS1-124K5.3 | TMEM126A | ACTA2-AS1 | CLIC1 | FLJ37O35 |
| PANK4 | RP11-172F4.2 | ERCC6 | ORC4 | LYAR | EIF2S2 |
| CIAO1 | MYO18A | AC097721.2 | CTD-2369P2.12 | CCL4 | C7orf55 |
| B4GALT4 | AFTPH | ACTR3C | NTAN1P2 | RP11-706O15.3 | RP11-83A24.1 |
| DBR1 | ADAMTS13 | AK3 | VPS4B | SDAD1P1 | TMEM205 |
| PDHB | ZXDC | RP11-461A8.1 | TOMM34 | SNHG4 | C4B-AS1 |
| C4A-AS1 | EPM2AIP1 | NUPL2 | GGA1 | ACAA2 | CDK17 |
| ZNF433 | CTD-2561B21.3 | MRPL57 | WNT8B | ATG3 | YBX1P1 |
| AUH | UBE2SP2 | INTS5 | PGD | ST13P15 | BRWD1 |
| POLD3 | GNPNAT1 | PP7080 | DERL2 | NHLRC4 | HINT3 |
| KB-1410C5.5 | RPL7P22 | MGAT2 | ZMYM6NB | RP11-849F2.9 | HCG4B |
| RMND1 | UBE2B | ANXA9 | RP11-977B10.2 | CH507-513H4.1 | CH507-528H12.1 |
| GRAMD1B | TRIM68 | TMEM179B | RP3-425C14.4 | RP11-417L19.5 | AUP1 |
| ADAT2 | RP11-417N10.4 | C15orf62 | RP11-855A2.2 | LINC01125 | RP11-1000B6.7 |
| FAM124A | ARMCX5-GPRASP2 | GALNT3 | STX18 | NMD3 | C19orf25 |
| RP11-28G8.1 | TSPAN12 | IGHV4-61 | C9orf78 | ZNF775 | TPD52 |
| KIAA2026 | PRKAR2B | SARAF | HAUS6P3 | PIGP | NPM1 |
| MT-ATP6 | CTC-260F20.3 | GALNT11 | CDC37L1 | RP11-460N11.2 | RP11-270C12.3 |
| RP11-500G22.5 | NOL9 | WDR19 | ADGRL4 | RP11-203B7.2 | PHLDB1 |
| PFKFB1 | CCDC136 | BCAT2 | RP3-402G11.27 | RP11-343L5.2 | PPIL4 |
| MNS1 | TBC1D31 | CENPC | RFXANK | DGCR6 | RP1-197B17.4 |
| CHMP5 | NUBP2 | CTU2 | ANKLE1 | HERC2P2 | POM121L9P |
| BAG4 | TRDV3 | TTC33 | SLC2A1 | ZNF597 | TMEM222 |
| SLC41A2 | RP11-234B24.6 | TMEM38B | BTBD18 | TBC1D3I | KLHL12 |
| POLG2 | MT-ND6 | LAMB1 | RP11-702F3.3 | FDX1P1 | USP36 |
| RP11-526I2.1 | Sep-08 | AP5Z1 | MRPL42 | POLK | MRPL35 |
| RP11-253M7.1 | SETD8 | THAP1 | OPA1 | RAB1B | AC034243.1 |
| RP5-1009E24.8 | IMMP2L | SBDSP1 | RP3-325F22.5 | PHOSPHO1 | SCOC-AS1 |
| DNAJC8 | OVCA2 | RP11-474P2.7 | B4GALT1-AS1 | IL17C | EIF3LP2 |
| AKR7A2P1 | MT-ND2 | RP1-39G22.4 | XXbac- | IL15 | ZNF674 |
| | | | BPG249D20.9 | | |
| NUP58 | STK24 | METRN | MASTL | RPL31P49 | RP11-382A20.1 |
| RP13-554M15.7 | SERBP1 | C2orf88 | ZNF138 | TRAPPC10 | TUBAP2 |
| DNPH1 | C3orf58 | RNF11 | RP11-932O9.9 | FBXO24 | HTR2B |
| RP11-521C22.2 | DCLRE1C | TRMT10B | DSP | VN1R108P | RP11-20I23.1 |
| PEG13 | TEKT5 | CH507-154B10.2 | RP11-574E24.3 | PQLC1 | GOLGA8K |
| SYS1 | RN7SL431P | CTD-2020K17.3 | NAP1L4P1 | RIF1 | MTRF1LP1 |
| SRXN1 | RP11-333E1.1 | PROCA1 | RP11-567G24.1 | SYCE2 | TECPR1 |
| CENPO | RP11-135N5.3 | AP001469.9 | DUX4L50 | MRPL39 | TREX1 |
| TMEM164 | RPRD1B | RP11-837J7.4 | COG2 | PRR26 | PSMG3 |
| DTX2P1 | DOCK7 | RP11-456J20.1 | TUBG1 | ABCC5-AS1 | OTUD3 |
| EED | RAD51D | PREB | RP11-513I15.6 | PRR23D2 | ERP44 |
| MAPKAPK3 | RPS10L | EXOSC7 | CTA-390C10.9 | TMPRSS9 | RP11-303E16.2 |
| NOTCH4 | PCID2 | SLC9A3R2 | DNAJC3 | NDUFB4 | MXI1 |
| SUN2 | MPHOSPH8 | LINC01160 | DPP7 | EEF1A1P13 | DPH5 |
| PIGZ | CTD-2095E4.3 | TMEM63B | POGLUT1 | MCCC1 | RP11-42O15.3 |
| HSPA9 | RP11-127B20.3 | LRRC57 | MYOM1 | NLRP6 | SPCS1 |
| ERGIC1 | SCRIB | AXIN1 | PNO1 | FGF22 | CTD-237614.1 |
| PSME3 | RP11-493E12.2 | MPLKIP | ADGRF5P1 | RHOA | ZNF264 |
| ABHD17C | MRPL49 | POLR2F | MAGEA2 | ATP6V1D | CLDN23 |
| TRMT5 | TOR1A | CIAPIN1 | SLC25A51 | POTEE | IRAK2 |
| RSPRY1 | RPL29 | MRPS33 | RP11-356J5.13 | SH3GLB2 | REST |
| AC005523.2 | MPZL2 | SPTY2D1-AS1 | HMGB3P24 | OAS2 | ZFHX3 |
| PPA1 | GEMIN6 | LL0XNC01-116E7.5 | RP11-477D19.2 | FIS1 | PRKAR2A |
| RP11-288I21.1 | EEF1A1P14 | TGDS | FMR1 | TRIM69 | DENND4C |
| MMS22L | P2RX5 | WDR18 | TMED4 | RP11-74J13.9 | AP000487.6 |
| RP11-690I21.2 | NPHP3 | RNPEPL1 | ACTR2 | RP11-554J4.1 | PPM1A |
| FRAT1 | DHRS7B | RP11-355O1.11 | RP11-465B22.3 | CTC-523E23.3 | RP11-50D9.1 |
| NDUFB9 | TMLHE | DNAAF5 | MVB12A | POC1B | AC010883.5 |
| HSPA2 | TMCO6 | RP11-932O9.8 | C17orf75 | APCDD1L-AS1 | FOXN3-AS1 |
| KMT2D | DCAF13P1 | CTD-3092A11.2 | RUNX3 | MED14OS | C5orf51 |
| RP3-412A9.16 | IGKV2-30 | ZNF215 | RP11-667M19.2 | PCNP | EMC9 |
| REXO1 | CCDC25 | ZNF395 | NEK3 | C1QBP | FAM185BP |
| CPLX1 | LA16c-390H2.4 | BAIAP2-AS1 | MACC1 | GS1-124K5.12 | RP11-713N11.6 |
| RPL7P32 | AC010504.2 | FBXO47 | IRAK4 | ERAL1 | VPS37A |
| PRR23D1 | ECHS1 | LINC00674 | PRDX5 | RANBP10 | HECTD4 |
| AKAP11 | AP001619.2 | XPO6 | SLC25A37 | RP11-1000B6.8 | AC137932.5 |
| MYCBP2-AS1 | LINC00886 | MRE11A | CHERP | SETD1B | PMS1 |
| RP11-259N19.1 | ZFP91-CNTF | PML | TLE3 | PTPRH | PIGT |
| INTS4P2 | RP11-379K17.12 | RP11-295P9.3 | RP11-274B18.4 | RP4-816N1.6 | SLC11A2 |
| RP11-16P6.1 | RP11-25K19.1 | NUP37 | ZNF66 | RP11-401N16.2 | DISC1FP1 |
| ZNF445 | RP11-120D5.1 | OPA3 | PHKA2-AS1 | ATP5C1 | RP11-408A13.4 |
| RP11-30K9.6 | C6orf106 | NNT-AS1 | EEF2KMT | WDR59 | EIF2S2P4 |
| VPS37B | PPP1CA | GFER | RPS27L | RP11-700P18.1 | AKAP17BP |
| RNF216P1 | MGA | TSR3 | RP11-567I13.1 | ADCY3 | GLI1 |
| TBCE | RP11-734K2.4 | RP11-19P22.8 | RP11-63A1.1 | MT-ND3 | GUSBP1 |
| NAALAD2 | CTD-3088G3.4 | SPG11 | KCNQ5 | HAT1 | USE1 |
| RNGTT | ESF1 | STOML2 | C6orf3 | MMADHC | RPS20P33 |
| CUL4A | TMEM52 | FTL | KCNJ14 | RP11-298I3.6 | CTC-756D1.1 |
| SMN2 | CNTF | USP20 | TSG101 | RP11-128N14.4 | SEC62 |
| RRN3P2 | EPB42 | RRP15 | BTK | GPR75 | ZNF747 |
| AVL9 | LA16c-380F5.3 | COQ10B | RRS1 | BCAS2 | GOLPH3 |
| HSPD1 | RP11-468E2.10 | PPT1 | MTND1P23 | CA5BP1 | POTEC |
| MRPS17 | CTC-260E6.3 | RP11-666A8.12 | SNX19 | RP11-378J18.9 | RP11-867G23.8 |
| CTC-398G3.1 | ERN1 | SGIP1 | ANAPC13 | TMEM147 | AC092580.1 |
| OR13A1 | DCAF17 | RP1-266L20.2 | SPDYE1 | HNRNPA3P10 | AC113189.5 |
| AL928768.3 | ZDHHC6 | TMEM104 | GRPEL1 | ANP32BP1 | HOXA4 |
| RP11-192H23.8 | PTGER4 | RP11-326C3.11 | ADD1 | LINC01315 | RP11-435O5.5 |
| CCNT1 | ZNF500 | ABCF1 | RP11-266L9.4 | NDUFA6 | GINS1 |
| AASDHPPT | TSR1 | MKRN3 | FAAP100 | SLC38A10 | SLC26A6 |
| RGS17P1 | RNF123 | RP11-72I8.1 | UBE2R2-AS1 | ZSCAN30 | DFFB |
| AOC3 | ATP1B3-AS1 | EFCAB14 | AC022182.2 | HMG20A | CEP152 |
| INO80D | AGFG2 | GEMIN8P4 | WASH3P | PNPO | TBC1D1 |
| TIMM9 | NUS1P1 | RPL7AP66 | B3GNT4 | FTSJ1 | PASK |
| CDK5 | HAL | FYB | HMGN3-AS1 | AGAP6 | RP11-247I13.3 |
| SRM | RP11-599B13.3 | SAP30BP | AF230666.2 | ACTG1P24 | ENPP1 |
| NSFP1 | LTN1 | TMEM80 | TRIM25 | RP3-465N24.5 | COA3 |
| B4GALT2 | TIMM8B | VWA8-AS1 | UBE2M | FBXW11 | HLA-A |
| OSGEP | ACBD5 | RP11-216B9.9 | C15orf40 | ARHGAP11B | PYCARD |
| RP11-465N4.5 | HINFP | MGME1 | CTD-2545G14.7 | RP11-378E13.3 | RPL5P34 |
| RP11-81H14.2 | RP11-855A2.1 | APTX | MRPL33 | RP11-15A1.7 | PTGES3 |
| DNAJB5 | AC087294.2 | ARF5 | OSBPL8 | RNPS1 | RP11-1072A3.3 |
| PANK3 | RWDD1 | Metazoa_SRP | IRF2BP2 | CBX3P2 | CDKL5 |
| RP11-525G13.2 | CEP68 | SEPSECS | JAK3 | TIMM50 | FAM120A |
| MLC1 | NUDT16L1 | DCAF13P3 | YAP1 | HCP5B | SMS |
| SLC35E1 | SIRT7 | ZNF491 | SPDYE5 | NABP2 | ZNF367 |
| SP2-AS1 | URB1 | VARS2 | CENPT | TAF1 | DNPEP |
| CRIPAK | UBOX5 | RP5-1185I7.1 | CCDC62 | RP11-304F15.3 | REL |
| CEP95 | AIG1 | MAP3K12 | UQCRFS1 | SIGMAR1 | RP5-1160K1.6 |
| RP11-293M10.6 | IFNB1 | NHP2P1 | DPP3 | STK32C | RP11-295D4.3 |
| RP11-476C8.2 | NDUFAF2 | MRPS30 | RPSAP41 | PANK1 | UNG |
| EFCAB11 | LLGL2 | PDCD10 | ST14 | TMEM191A | CD53 |
| ACLY | COA4 | CTD-2350C19.2 | SPIN2B | AC008746.5 | TMSB4XP4 |
| MED4-AS1 | MTG2 | RAPGEF2 | TMEM184C | PIM3 | IGLV6-57 |
| ARPC4 | TUBD1 | NEFH | MT-ND5 | RP13-104F24.3 | HKR1 |
| RP11-130L8.1 | GDAP1 | SNHG17 | GLS | ZNF829 | FARS2 |
| FAM208B | LRRC37A17P | RGS19 | SERPINB1 | REEP2 | MTERF1 |
| CORT | AC005154.6 | ARFGEF2 | FLJ20021 | EDEM2 | ANKRD26P1 |
| CITF22-49E9.3 | IGKV2-24 | IFT122 | BMP8B | MFNG | PTBP1P |
| TYW5 | CETN2 | RP13-672B3.2 | STAG3L4 | SMUG1 | CMPK1 |
| PRPF39 | PER3 | ARID4B | RP11-732A19.8 | AHI1 | PVRIG |
| FAM69B | EIF3C | RP1-101K10.6 | ZP3 | NEMP2 | AC026150.8 |
| MRS2 | ATP5G1 | CISD1 | RP11-706O15.7 | ZNF800 | NAIP |
| SLIT1 | IGLV3-1 | COA5 | CTD-3092A11.1 | RP1-191J18.66 | AP001056.1 |
| LRRC28 | INTS2 | TSPYL5 | RP11-419C5.2 | NUP88 | FP325331.1 |
| ZRANB2-AS1 | CTD-2517M22.17 | ZNF805 | CCDC102A | SNAP23 | C5orf15 |
| PHF11 | TIMM44 | RP1-228H13.5 | PTPN13 | NARS | PNLDC1 |
| GXYLT1 | EID3 | UPF1 | HCG2040054 | RP11-321A17.6 | HAUS3 |
| C5orf24 | RP11-240G22.5 | ZFPL1 | MRPL50 | TRIP10 | LEPROT |
| PCP4L1 | PKDREJ | FTH1 | ZNF197 | ANKRA2 | ZNF783 |
| TYROBP | CTB-193M12.5 | SCYL1 | VN1R81P | PLA2G4C | SUCLG2 |
| LYSMD3 | OGDH | DNTT | EFCAB10 | TMEM38A | ZNF362 |
| TRNT1 | TAPT1-AS1 | CELF5 | RP11-343H19.1 | ITGAE | MIR3179-3 |
| PURB | PDE6A | CARHSP1 | RP11-458D21.1 | MBLAC1 | MTND5P28 |
| OTUD6B-AS1 | RABGAP1 | TXNL4B | RP11-79O8.1 | ZNF815P | PLA2G10 |
| GEMIN7 | CHEK1 | AP5M1 | AC092338.5 | TCEB3-AS1 | ATP1A3 |
| USP19 | LRRTM4 | MAP4K2 | U47924.27 | SGPP2 | PCSK7 |
| KPNA1 | FAR2P3 | SDHAF2 | GMNN | EXTL2 | PA2G4P1 |
| AMIGO3 | SMIM14 | PRPF19 | FAM32A | RP13-638C3.4 | HEXIM2 |
| ZCCHC4 | RPL35 | ZNF19 | FGD1 | GAMT | RP1-228P16.8 |
| NDE1 | RP11-109G23.3 | KCNJ13 | ANKHD1-EIF4EBP3 | PRRT4 | TANC1 |
| EIF5B | RP11-318A15.2 | PPDPF | RNFT1 | RAC1 | SLC25A1 |
| RP5-855D21.3 | MRPL16 | DAG1 | RP11-1167A19.2 | RP11-231I16.1 | RP4-671O14.5 |
| FASTK | RP11-640I15.1 | LRRIQ4 | RP11-267M23.1 | CLSTN1 | FAM120AOS |
| CCDC85B | AGAP2-AS1 | TMED7 | ARPC3 | PHF20 | RP11-429D19.1 |
| NSL1 | ST3GAL4-AS1 | WDR5 | RAD23A | MTND6P4 | ARMC10 |
| PEF1 | XXyac-YRM2039.3 | DHFRL1 | EIF2D | MRPL23 | ALG13 |
| MRPL44 | RAET1E | HSPBP1 | EPHB4 | MAFF | MAMDC4 |
| NME4 | C20orf24 | CREB5 | RP13-104F24.1 | BOLA2 | FCHO2 |
| FAM129A | HTRA4 | RPAP2 | ARAP1-AS1 | HYKK | HMGCR |
| TMEM44 | DCTN2 | VDAC1P8 | IARS2 | TLK2P1 | RP11-545I5.3 |
| RP11-127B20.2 | ARPC2 | CCT6P3 | SAMSN1 | MFAP3 | NOSTRIN |
| FANCA | RP11-163E9.2 | LINC00865 | OGFRP1 | CISD2 | GGT6 |
| ZNF219 | TIPARP-AS1 | USP40 | RP11-266L9.3 | MTHFD1L | RP11-66N11.8 |
| RP11-638I2.2 | ZNF391 | SUN1 | EARS2 | CD68 | NOC2L |
| RP3-408N23.4 | RP11-313P13.4 | FYTTD1 | CTF1 | TIRAP | APOBEC3F |
| FAM223B | FAM223A | AC023590.1 | TM4SF1 | RAB44 | EXOSC6 |
| RYR3 | RP11-114N19.3 | HYI | REEP5 | CTD-2199O4.7 | EXT1 |
| CEBPZOS | RP11-229P13.2 | CHIC2 | RP11-398A8.1 | TOMM22 | AGBL5 |
| ZFYVE9 | TMEM39A | RP11-388C12.5 | PDCD6 | LYST | ATP6V1E1 |
| STAP1 | RBAK-RBAKDN | RP11-165F24.5 | AREG | PPIL2 | ZNF788 |
| METTL2A | TMEM9B-AS1 | PAQR5 | HSPD1P10 | PPP6C | BCKDK |
| PSMD7 | RP11-524O1.4 | PTPRJ | CH507-9B2.1 | KPTN | RAB14 |
| SPDYE2B | RP13-890H12.2 | SLC30A7 | POLR3K | MRPS31 | SHISA5 |
| SPIN2A | STXBP3 | TMEM106C | ZDHHC23 | DRG1 | CTD-3224K15.2 |
| ITGB7 | PALLD | AAMP | COG1 | C16orf91 | Metazoa_SRP |
| RHD | ELF4 | RP11-306G20.1 | RP11-159F24.3 | TMEM60 | BAG1 |
| AC083843.1 | S1PR2 | RP1-148H17.1 | RPSAP26 | SLC7A5 | KB-1507C5.4 |
| CTDNEP1 | SNAPC1 | BCAR1 | PSMA6P1 | AC012363.8 | EIF4BP7 |
| EP400NL | RNF222 | A2M-AS1 | RP11-738E22.3 | AC093627.9 | ZNRD1-AS1 |
| OACYLP | AC068831.15 | CSAG1 | AP000648.5 | ZNF514 | GRB2 |
| GBA2 | SAR1A | ZNF774 | LINC01225 | RP11-464F9.20 | CARNMT1 |
| AFG3L2 | APRT | ZNF768 | MEF2A | ESD | AP000295.10 |
| RP4-635E18.7 | MATN2 | SPOP | ANKRD36C | GPX7 | AP000769.1 |
| RP11-555K12.1 | RP11-337N6.3 | MCCC2 | PMS2P1 | RP5-1039K5.19 | ATP5G3 |
| SCFD1 | SAP25 | WNT5A | RP11-580I16.2 | SLC46A3 | SLC23A2 |
| STXBP5L | INADL | ENDOG | NIPSNAP3A | KIAA1462 | TLR1 |
| TMEM109 | CTA-414D7.1 | ZNF589 | DPM2 | NKIRAS2 | NR2C2 |
| NINJ2 | PGAM4 | GUK1 | SCAND1 | MAPT | HIC1 |
| IMPG1 | LINC01063 | ZBTB34 | AC005822.1 | AC098820.3 | ERLEC1 |
| NCAN | RPL36AL | AP000349.2 | DNAH2 | TMEM249 | RP5-991G20.1 |
| RP11-803D5.1 | EXD1 | ALKBH1 | FAM87A | SUCLA2 | RP11-567M16.6 |
| TIMM22 | PRKACB | RASIP1 | C2orf50 | HEMK1 | DCAF4L1 |
| DNAJC27 | RP4-530I15.9 | NIPA2 | RN7SL333P | RP11-552F3.4 | COX19 |
| ABI3BP | RP11-507K2.6 | MUSTN1 | SLC38A9 | ZC3H12A | NTPCR |
| ZNF185 | LIN7C | AC009501.4 | NRSN2-AS1 | TAS2R14 | FAM72B |
| ANXA2R | MBD1 | C22orf39 | DNM1L | RP11-574F11.3 | PDSS1 |
| TGFBR1 | NRBF2P5 | COQ2 | RP13-192B19.2 | RP11-358N2.2 | KLHL28 |
| VDAC2 | LILRA6 | AC135050.5 | PSAT1 | C14orf37 | CTD-2622I13.3 |
| CCNB1 | MYDGF | RP11-435O5.6 | GCDH | UHRF1 | ACTG1P20 |
| RFC2 | CTC-451P13.1 | GPATCH8 | STK10 | MYBPC3 | RAD51-AS1 |
| F8 | C3orf17 | GRIN3A | AC006116.17 | SPTLC1 | ZNF680 |
| INSM2 | RP11-255E6.6 | RP11-192H23.4 | RP11-799B12.2 | KHDC1L | WIPI2 |
| MRPS5 | PSME2P2 | HOOK2 | TRIM61 | LMBRD2 | LRRC37A4P |
| AC004893.10 | CATSPERG | Mar-02 | AC024560.3 | RBAK | SLC9A1 |
| KIAA1328 | GJC3 | MRPL47 | RNASE4 | LYRM5 | IMPDH2 |
| RP11-50I19.2 | FOXP3 | NDUFAF7 | RP11-109N23.4 | TMEM50A | RP11-384K6.8 |
| RP11-356I2.4 | CBFB | RWDD4P1 | RP11-547D23.1 | CD55 | RP11-474L23.3 |
| RAB5A | RALA | HHEX | EEF1A1P12 | MRPL20 | TMIGD1 |
| RP11-266L9.1 | RP11-697E2.6 | CCDC124 | GOT2 | AGL | PIK3C3 |
| CYP51A1-AS1 | CTD-2553L13.10 | AC005785.2 | AC027612.6 | RP11-316M20.1 | KLHL3 |
| AC104653.1 | RP11-488L18.8 | SPTBN1 | LRRC37B | ANXA6 | METTL22 |
| HMOX2 | RPL9P25 | SEH1L | SLC15A2 | AP001059.5 | FAM200A |
| ANAPC1P1 | RP11-778J15.1 | SVILP1 | RNF166 | C12orf10 | FAM135A |
| ARAP2 | DAB2IP | METTL14 | LNPEP | MYL9 | SPDYE11 |
| PAXIP1-AS2 | DUSP2 | NKAP | TMEM218 | C16orf70 | IL32 |
| PFKFB2 | UBE2T | TMEM43 | RP3-329A5.8 | SULT1A2 | ZNF750 |
| PRIMPOL | NTNG2 | GEN1 | ERVFRD-3 | ARPC1B | RP11-697E2.9 |
| SRRM2 | RP11-25L3.3 | DNAH1 | LINC00355 | KIF3B | FAM188B |
| RP11-496I9.1 | SIN3A | RP11-277P12.20 | RP11-345J4.1 | PARS2 | MTPAP |
| STAR | NADK2-AS1 | BEST1 | CEP44 | SURF4 | AF131215.4 |
| AC012123.1 | SLC5A10 | RP11-286N22.14 | RUNDC3B | LINC01337 | TXNRD1 |
| PTGES2-AS1 | SUCLG1 | VMP1 | MAGEA12 | POU5F1P6 | RPL18AP7 |
| RASSF1 | THBS2 | WRB | MTCO2P12 | AP000437.3 | FAM98C |
| COQ4 | RP11-596C23.6 | PRSS27 | ARL16 | MRPS36 | RRH |
| CACTIN | C4orf26 | RRP1 | MROH8 | CTB-152G17.6 | METTL6 |
| CEP120 | COX7A2 | RCC1 | CCDC85C | RP11-260M2.1 | AC092667.2 |
| ALDH3A2 | IGLV1-47 | HTR7P1 | LEPROTL1 | ZNF787 | TMEM208 |
| CHID1 | WDR36 | EFHB | SELO | KB-1440D3.13 | HLA-F |
| PDE4A | RPL23AP10 | RAB7A | ZFY-AS1 | PGF | SHMT1 |
| RBM44 | MED15 | OSGEPL1-AS1 | ZSCAN29 | RP11-490K7.4 | UCHL5 |
| RP11-242C19.2 | RP4-569M23.4 | RP11-6N17.3 | RP3-508I15.9 | RARS2 | ATP13A3 |
| Sep-15 | CHMP6 | MTMR7 | TBC1D29 | TMX3 | MPP7 |
| CRK | RP11-138I1.4 | PAPD4 | BSCL2 | CTD-2587H24.10 | RPPH1 |
| TOR2A | STARD3 | C5AR1 | GS1-393G12.13 | CTC-457L16.1 | TGFBRAP1 |
| AC135048.13 | RP1-292L20.3 | ZNF724P | RP11-798G7.6 | RNF4 | TPRG1L |
| PDIA3 | SNHG20 | AC105760.2 | PHB2 | COX4I1 | FAM49B |
| CEP192 | HSPA9P1 | ARPC5L | ZCRB1 | ZNF319 | IER5L |
| SQLE | ZNF317 | CFAP43 | TYRO3P | KANSL1 | SH3RF1 |
| RP5-908M14.10 | SMAD5-AS1 | ELK4 | RP11-10O17.1 | HSPA1B | LINC00431 |
| TPST2 | NUDT15 | ASPSCR1 | NPEPPS | ARAP1 | ZNF773 |
| NAV1 | CYB5R4 | SC5D | KPNA4 | HEY1 | RP11-474G23.1 |
| COTL1 | RP5-1014D13.2 | DAGLB | CTD-2342J14.6 | IDI1 | AC116366.6 |
| URGCP | ABTB2 | CEPT1 | MLX | SMG1P5 | UBE2L3 |
| SLC10A1 | ZSCAN25 | RFK | WDR11-AS1 | POLE2 | CHST11 |
| GADD45GIP1 | HSPB11 | TOMM20P4 | HADHA | RP11-53I6.4 | CTC-277H1.7 |
| PTS | MRPL18 | TOP3B | SYT16 | MRPS18C | LMBRD1 |
| TTC30B | RP13-977J11.2 | MUTYH | PSMA3 | TMOD4 | MCC |
| CTD-3126B10.2 | AP4B1 | RP11-81A1.3 | CUL3 | SLC25A17 | POLDIP2 |
| ELOF1 | WDR54 | RP11-861E21.2 | AC009473.1 | RP11-977G19.5 | IRAK1 |
| WNT10A | RP11-121M22.1 | DKFZp779M0652 | RP11-1072C15.4 | RP11-51O6.1 | THAP7 |
| IGHA2 | VRK2 | STRADA | ENO2 | SLC41A3 | ST3GAL1P1 |
| ITPRIPL1 | RP11-317N8.5 | ACSS2 | RSAD2 | RBM3 | RPS11P6 |
| ZNF44 | DEPDC1 | MAB21L1 | SMIM11B | ERVH48-1 | NDUFA5 |
| RP11-504P24.9 | TMEM120A | RP11-297N6.4 | RP11-307E17.8 | MT-ND1 | PCYT1A |
| GPALPP1 | C1orf162 | WWC1 | RP1-265C24.5 | CTB-39G8.3 | PCDHB2 |
| ERO1A | RP11-84G21.1 | PTPN1 | RP11-403P17.4 | CD74 | RRP1B |
| RP11-408A13.3 | AC010468.1 | MKRN2OS | LINC01001 | AC002310.10 | FAM81A |
| PTTG1IP | GPX1 | GUCD1 | C1orf35 | CTD-2270N23.1 | DCP1A |
| RTTN | IDUA | RP5-1041C10.3 | PSME2 | NT5C3B | SLC5A11 |
| CDC34 | THAP9-AS1 | L3MBTL2 | DDX28 | AKAP10 | SRD5A3-AS1 |
| ATP5O | ADAM21 | GET4 | C7orf31 | HPD | GACAT2 |
| PRELID1 | C1QTNF9B-AS1 | RPL23AP24 | SRRM1 | OIP5-AS1 | IL12RB2 |
| RILPL1 | TMEM70 | ATP6V0C | CHRFAM7A | CYHR1 | CHAF1B |
| OS9 | ARHGEF40 | OSBPL10 | SLCO4A1-AS1 | RP11-384F7.1 | A1BG-AS1 |
| C22orf15 | LRG1 | RTCA | FBLN1 | TBC1D25 | CTC-487M23.6 |
| TSNAX | PLP2 | BTN1A1 | RN7SL608P | TMEM259 | WDR92 |
| MROH2A | UGDH-AS1 | RHOQ | PSKH1 | XRCC6BP1 | RP4-758J24.6 |
| LYRM1 | SNX20 | CDC42SE2 | LETM1 | SMIM4 | RP11-66B24.4 |
| RP11-589M4.1 | CD8A | SLC25A14 | ARHGAP4 | CTC-453G23.7 | ZNF337-AS1 |
| SCARF2 | AP1S3 | SNX5 | RP11-642A1.2 | KIAA1715 | CACTIN-AS1 |
| QARS | UBTD2 | CTB-75G16.1 | KLC1 | GOSR2 | SPINT2 |
| UNC50 | TRAPPC2P1 | CAMTA2 | TYW3 | CNBD2 | ANKZF1 |
| CFD | PLEKHA2 | ACVR1 | SLC25A23 | FAR1 | ALG6 |
| RP11-342M1.3 | CD3EAP | RP11-430G17.3 | ZNF547 | RP11-342K2.1 | MMGT1 |
| KRT9 | BRMS1 | SLC27A1 | COX11 | NEK9 | CTD-2650P22.1 |
| PKN1 | RP11-831H9.11 | STYXL1 | RP1-199J3.7 | EPS15L1 | CEP170B |
| CAPNS1 | ARF6 | COL4A3BP | SBDS | PA2G4 | CBR4 |
| CUL5 | PPP1CC | RCAN3 | INPP5B | CEP162 | CTC-512J14.5 |
| AGAP9 | AMD1 | CBWD1 | CTB-50E14.5 | ZNF302 | PLXDC1 |
| PTGES3L | MINOS1 | PYROXD1 | SLC38A7 | NEURL2 | CXorf40A |
| SRP68 | ANAPC11 | PACS1 | PTGES3L-AARSD1 | XXbac- | TBC1D12 |
| | | | | BPG157A10.21 | |
| TAF1A | ACSF2 | PET100 | DARS | RP1-309F20.3 | AIFM1 |
| MAST1 | LRIF1 | FAM122A | RP11-822E23.8 | HOXC9 | FN3KRP |
| STAM2 | FLNB-AS1 | RIOK3 | UTP11L | MAN1B1 | PGLS |
| RIPK2 | UBE2V2 | RP11-380O24.1 | CRIPT | RP11-93G5.1 | MSL2 |
| CCZ1 | WDR24 | RP11-166A12.1 | ZBTB16 | TTC28-AS1 | RP11-506M12.1 |
| OTUD4P1 | FAM13B | RP11-534L6.2 | RP11-453E17.3 | KL | CACNB3 |
| HS2ST1 | HIGD1AP1 | VPS11 | MACF1 | RP11-1149O23.4 | AES |
| CEBPB | ZFR | SFXN1 | RP11-936I5.1 | ZMYM6 | SUGT1 |
| LCA5L | CRAT | AP2S1 | FBXO2 | MMP15 | DNAAF2 |
| RP5-1099D15.1 | SESN2 | MIR155HG | USP30-AS1 | ILVBL | AC093690.1 |
| TPMT | EGFL7 | WDR25 | C6orf203 | MIEF2 | FLJ30679 |
| SPDYE9P | CTC-756D1.2 | RP11-950C14.3 | RP11-481C4.2 | BNIP3P1 | RASSF5 |
| ELOVL5 | RP11-381E24.4 | MRVI1 | RP11-337N6.1 | CASC1 | SMIM19 |
| AL133243.2 | CXorf56 | NEK10 | PPP6R1 | SOCS5P4 | RP11-982M15.2 |
| RP11-439E19.7 | TAX1BP3 | MRPL11 | RP11-394O4.3 | SRPR | TMEM11 |
| ANAPC16 | ZSWIM8-AS1 | SLC1A4 | NDUFA6-AS1 | ZNF346-IT1 | SPRYD7 |
| TTLL1 | RP11-867G23.3 | AC010976.2 | DCUN1D1 | C19orf70 | FANCB |
| PCDHB11 | KANSL2 | JPH3 | IP6K1 | TGFB2 | ETF1 |
| CCL28 | PCYT2 | AAGAB | CTD-2196E14.7 | EFR3A | NFS1 |
| CTD-2313J17.6 | MPP6 | RP11-378J18.8 | SCO2 | FUCA1 | CTD-2278110.4 |
| NAA40 | ZBTB47 | RP11-479G22.8 | DIABLO | GPN3 | MTND4P26 |
| CNST | MYCBP2 | BRI3BP | SLC25A5P1 | CYB5B | RP11-392O17.2 |
| FAM166B | TNIP1 | TMUB2 | CCDC86 | EPSTI1 | LA16c-306A4.1 |
| CTD-2184D3.5 | ALOX12 | ASB14 | AC136289.1 | CTD-2358C21.5 | CCDC18 |
| SLC7A6 | RGS18 | RP11-251G23.5 | DAD1 | PPP3CC | TBC1D19 |
| FAM229A | FRMD5 | CASC4P1 | CDADC1 | NARF | SNCB |
| DNAJC28 | RP11-473M20.9 | MEFV | EML6 | AC000123.4 | LINC00957 |
| DNM1 | NUBPL | CTA-984G1.5 | MRPS35 | C6orf120 | C11orf71 |
| RP11-18H7.1 | CTB-92J24.2 | CTD-2330K9.3 | RP11-505K9.1 | RP11-77H9.8 | ANKRD28 |
| RP11-381E24.1 | BPTF | STK25 | AP001962.3 | TMEM253 | SPDYE3 |
| ZNF584 | MALSU1 | MBLAC2 | KAZN | ATP5H | EEA1 |
| TBCCD1 | PMS2P4 | BIRC5 | KIAA1919 | XX-FW83563B9.5 | AHCY |
| AC005519.4 | RP11-345J4.3 | RP11-434P11.2 | MRPL22 | TUBGCP6 | AL592183.1 |
| KIRREL2 | FAM27E3 | PMM1 | RP11-958N24.1 | RP11-371E8.4 | RP11-15J10.1 |
| C16orf71 | SLC25A13 | PCIF1 | RALGDS | BAIAP2L2 | RP11-158H5.8 |
| AC002467.7 | PDAP1 | UFM1 | SETDB2 | MEN1 | HOXB7 |
| DIS3L | RP11-7O11.3 | RP11-428G5.5 | OBSCN | AASDH | C10orf105 |
| C1orf233 | ANKRD20A5P | DTWD1 | SIRT3 | LINC01424 | TPTE2P5 |
| LA16c-380H5.5 | RP11-336A10.2 | ATG4D | TUBA1C | GSR | PLIN3 |
| ZNF761 | HNRNPA3P9 | TMEM87A | DLAT | CTD-2012K14.7 | PTRF |
| ITPR3 | STX16 | RP11-34P13.14 | RN7SL1 | ANO7P1 | MVK |
| CCDC59 | GOLGA8B | GYS1 | CEP104 | PCMTD2 | SLC7A1 |
| ANKRD13A | FAM174A | RP11-57H14.2 | M6PR | CDC42P6 | GRPEL2 |
| XRCC1 | AC007192.4 | CTBS | CEP83-AS1 | JUP | TBRG1 |
| SPOCK1 | NIP7 | RNF113A | MARCH1 | TMEM256 | QTRTD1 |
| RP11-314A20.2 | TAF12 | LRCH1 | NUDT18 | CTNS | ZNF638-IT1 |
| BSN | PPIB | PMS2P7 | C9orf152 | POP4 | TTC41P |
| RP1-55C23.7 | FUT10 | CANT1 | AMZ2 | NEBL | AC005618.6 |
| MDP1 | DGKE | WDR78 | ATL3 | GCSHP5 | CTRL |
| ATPIF1 | MAVS | ATP5F1 | SLC4A2 | ZMAT3 | PGBD4 |
| MAP2K1 | TCIRG1 | TIMM13 | ABR | SLC25A4 | BNIP2 |
| FRS2 | LRRC59 | PMPCA | SP110 | SPCS3 | HMMR |
| GPD1L | ERP29 | RP11-73O6.3 | PLEKHG4 | MIR181A2HG | CTC-444N24.8 |
| RP11-399B17.1 | LY75 | DDX10 | RP4-784A16.5 | AC002115.9 | RPL9P29 |
| PCDHGA11 | CMC2 | RP11-111M22.4 | UHRF2 | RP3-395M20.8 | CTD-2270P14.1 |
| RPN2 | NKTR | ERCC6L2 | ANKRD61 | SELM | CUL7 |
| DTWD2 | NCR3 | ARIH2OS | GOLGA6L5P | HESX1 | PARP9 |
| ZNF286A | LINC00299 | LYPLAL1 | RP11-325N19.3 | TBC1D17 | ARHGAP24 |
| IFIH1 | MTHFD2P7 | SNORD22 | GTF2H1 | SARS2 | DAND5 |
| TMOD1 | RPRD1A | JRKL | AC004893.11 | REEP1 | TPCN1 |
| RP5-857K21.4 | EIF2AK2 | OR7E38P | MRPL32 | ARMC2 | RP11-158H5.7 |
| C9orf142 | RP11-175P13.3 | WDR31 | RP11-326C3.15 | ARV1 | RP11-96K19.5 |
| RP11-1072A3.4 | CHCHD4 | DENR | EIF1AX | TAS2R15P | PRCP |
| PUSL1 | CTD-3126B10.5 | ZNF268 | LPIN1 | PRDX4 | MRPS22 |
| MTATP8P2 | ERVK3-1 | PPP1R15B | MOCS2 | GNPTG | ZFYVE26 |
| AGFG1 | RNASEK-C17orf49 | RP11-299P2.1 | DNAJC6 | ADGRD1 | TUBA8 |
| RP11-960L18.1 | ISCA1P1 | PTPRC | LINC01136 | RMDN3 | RP11-282O18.3 |
| NIPA1 | ATP5A1P5 | GATAD2A | CCDC39 | DIP2C | DMGDH |
| FBXL6 | CDC37L1-AS1 | RNF43 | USP53 | ARL2BP | ANKS6 |
| RP11-171I2.2 | CCND1 | TRDC | GRSF1 | N6AMT2 | C8orf82 |
| RP11-1149O23.2 | VWA7 | RP11-766F14.2 | CTC-492K19.4 | WDR7-OT1 | PBDC1 |
| SLIRP | SDR42E1 | SLFNL1 | KREMEN2 | RP11-307C12.11 | RPS7P1 |
| RP5-966M1.7 | PPT2 | XK | RPS26 | ANO7 | C12orf60 |
| TMEM50B | RP11-727A23.1 | DHRS12 | DPM1 | RBPMS | GCSH |
| GUSB | AC011330.13 | IGFBP2 | ATP6AP1 | MNX1-AS1 | AC068499.10 |
| CBWD5 | RP11-250H24.2 | FUCA2 | RP11-425D10.10 | NUS1 | IKBKG |
| DFNB59 | COL6A6 | CD320 | PPP1R3D | TCHP | TUFM |
| ECH1 | AAAS | GPNMB | DDX55 | MOB1A | TIMM17A |
| GSN-AS1 | CTC-265F19.3 | TECR | MFF | COX5A | IFRD1 |
| RP11-666O2.2 | STAG3L1 | THOC2 | BRIX1 | SLC12A3 | TMEM69 |
| DAP | PSMG3-AS1 | RP11-10C24.2 | EEF2K | RP11-793H13.8 | MCOLN2 |
| PTP4A2P1 | RP11-250B2.6 | SYS1-DBNDD2 | BCORL1 | HSPA8P8 | SLC14A1 |
| ZNF550 | GIPC2 | LRRC18 | RP11-658F2.8 | OMA1 | DERL3 |
| TIMM21 | RP11-729L2.2 | FGFRL1 | SUPV3L1 | CCDC7 | TSN |
| AL928654.7 | CTA-363E19.2 | HCAR1 | VWA9 | LARP4B | RP11-67L3.4 |
| RP11-819M15.1 | KDM4D | SPOPL | MTPN | C19orf52 | CHKA |
| PPIL3 | POLR2E | PES1 | RP11-326I11.5 | EMC10 | AC133528.2 |
| PLEKHA5 | RP6-109B7.2 | NDUFS7 | CLEC16A | XXbac- | RP11-298I3.1 |
| | | | | BPG299F13.14 | |
| NQO2 | BCDIN3D | PSD | HCG27 | CDKN2AIPNL | RP11-881M11.2 |
| NR4A1 | TMEM173 | RRS1-AS1 | POLR3G | DDT | PIP5KL1 |
| CINP | TTC30A | LINC00342 | KCNA6 | CACNB2 | CAMKK2 |
| TMEM30A | SNX18P27 | MRPL14 | ARFRP1 | RP4-785G19.2 | ZNF790 |
| SSH2 | PDRG1 | RP11-10E18.7 | ZNF324B | HN1L | CEBPE |
| MCTS1 | RP11-500G10.5 | SLC4A10 | CTB-25B13.5 | DCP1B | CFAP44 |
| NFKBIZ | ETAA1 | ANKDD1B | CTA-445C9.14 | ELP3 | RP11-383I23.2 |
| RP11-374F3.5 | RP11-574K11.5 | RP11-359P18.7 | SMC5-AS1 | CTD-2349P21.9 | MRPL13 |
| ZNF304 | AC087163.2 | TPRKB | RP11-66N24.4 | CD86 | RP11-831F12.3 |
| TFRC | AIMP2 | AURKAIP1 | CTC-543D15.8 | EIF4E1B | RP13-395E19.3 |
| IFIT2 | SLC39A14 | VASH2 | ALG8 | CTD-2555O16.4 | AP001505.10 |
| RP11-338K17.6 | RAB3A | LSMEM1 | RP11-219D15.3 | DDHD1 | RP11-563E2.2 |
| DVL1 | RP11-472B18.1 | ESRP1 | INPP1 | DENND1A | SPTSSA |
| DDX3X | RPF2 | NKX3-1 | AF131215.9 | IMPDH1 | KANK1 |
| DENND4A | RP11-191G24.1 | TOMM20 | CHURC1 | RP11-206L10.2 | RP11-347C12.1 |
| TMEM161B-AS1 | RP11-690D19.3 | PPARD | RP11-490G8.1 | ARMC12 | RP11-603J24.17 |
| TMEM59 | SH3D19 | NRN1 | RP11-447D11.3 | LYPLA1 | RP11-661A12.5 |
| MYBBP1A | CEP83 | RAB4A | ZBTB25 | CHPF | UBR2 |
| IRF7 | AC108676.1 | DEAF1 | C18orf21 | PSMD12 | KIF4B |
| DCTPP1 | HIST1H3J | PIGK | RPUSD3 | SLC35C2 | C5orf22 |
| RP1-29C18.8 | DGUOK-AS1 | VKORC1L1 | RPP40 | ALG9 | DNAJA3 |
| ATP1A1 | KHK | RNF141 | RPUSD4 | RP11-1348G14.4 | NR3C1 |
| IPO9 | RP11-752G15.10 | LOXL4 | RP11-212P7.2 | CKMT2 | PPIAP11 |
| FCER1G | ZSCAN31 | ABRACL | LL22NC03-86G7.1 | DNAJC5 | CEP290 |
| AREL1 | MCCC1-AS1 | KTN1 | ZNF124 | MRPS10 | PPTC7 |
| PHYHD1 | ZNF777 | MRPL46 | LINC01237 | CTD-2010I16.1 | DLD |
| OAT | RP11-453N18.1 | RP11-63A1.4 | C12orf76 | STMN3 | RECQL5 |
| UBAP2 | RP11-317B3.2 | POLR3F | CTD-3099C6.9 | JHDM1D-AS1 | CTD-3193O13.12 |
| DDX60 | CS | UBR3 | RP11-218F10.3 | CTD-2357A8.2 | NDUFB10 |
| RP11-767N6.2 | NAPRT | OSBPL3 | LAMTOR1 | ASIC3 | CTA-228A9.3 |
| CICP14 | UPF3A | TBL1Y | JDP2 | RPS3AP6 | COMMD4 |
| VGF | C11orf16 | HCG4P7 | RP11-421M1.8 | ATG4B | WDR6 |
| RP11-88E10.5 | BOLA3 | ELL | CTC-444N24.7 | MRPL12 | ESCO2 |
| PMM2 | FAM219B | ST13P18 | CTD-2231E14.8 | CTB-180A7.6 | ARL5A |
| TLL2 | RP11-347I19.7 | APOBEC3G | RP11-263K19.4 | RPL29P11 | RP11-136K14.1 |
| APMAP | THAP7-AS1 | MZT1 | UFL1 | TMEM131 | RP11-34P13.8 |
| USP25 | CTD-2349P21.3 | RPL7L1 | SDCCAG8 | ZNF839 | PNRC2 |
| ACTRT3 | RFC5 | RP5-1085F17.3 | FAM3A | SLC9A6 | DPAGT1 |
| URB1-AS1 | MPV17L2 | SURF1 | SLC25A38 | FOSL1 | NUCB2 |
| RP11-101E13.5 | CAPN1 | CES4A | DENND1B | PRORSD1P | ZNF837 |
| MRTO4 | AP001189.4 | PIDD1 | IFT57 | IZUMO4 | MAPK1IP1L |
| EMC1 | HOXA5 | RPL7P50 | SSFA2 | SH3GL1P2 | TMED8 |
| HSH2D | RPAP1 | RP11-342K6.1 | ZNF700 | USHBP1 | RRM2B |
| RP11-324I22.4 | CTAGE7P | CCNL2 | FRYL | TSC22D4 | RP11-110I1.12 |
| RP5-1042I8.7 | RP11-761I4.4 | CARD19 | RP11-840I19.3 | C1RL | STRN3 |
| CDK14 | HTATIP2 | EBPL | EBAG9 | CAB39P1 | PSPN |
| NSMF | TXNDC17 | CUEDC1 | SLC39A11 | ACOX1 | SLC35C1 |
| RRAS | NDUFC2 | ZNF84 | RP11-131H24.4 | NAGPA | CENPK |
| GCH1 | PRKXP1 | RP5-862P8.2 | PDE4C | MRPL53 | PIAS4 |
| RP11-802D6.1 | RP11-348P10.2 | THADA | RAET1K | RP4-775C13.1 | MIA3 |
| FAAP20 | RP11-505K9.5 | SLC35D1 | STARD9 | LST1 | GNL1 |
| TDRKH | EIF4A1P10 | RP13-210D15.8 | VMA21 | CFB | CASD1 |
| BRCA1 | ADAL | WARS | DGUOK | C3orf20 | EFCAB13 |
| WASF2 | HOXA13 | HOXA10 | TSACC | GAS8 | LSS |
| KCTD13 | MRPL38 | ATRAID | EIF2B4 | OXCT1 | RP13-516M14.8 |
| AGPAT4 | RP1-97D16.1 | SAMD12-AS1 | CNEP1R1 | CPT2 | SNRNP25 |
| FAM91A1 | LINC01215 | SAMD8 | SPIN3 | RP11-20O24.4 | TTN-AS1 |
| DHX38 | LIPE-AS1 | NOP14-AS1 | TTC39C-AS1 | RP11-400F19.6 | TRMT61A |
| TNFRSF10C | RP11-274B21.9 | RP11-474N24.6 | RTN3 | CLN6 | ADAM1B |
| TNKS2-AS1 | IDH3G | BCKDHB | FAM72A | SDCCAG3P1 | AC023271.1 |
| AC005786.7 | ZMPSTE24 | CCDC163P | NOP56 | C1orf122 | RBM47 |
| RP4-622L5.2 | CTD-2521M24.8 | ABCC4 | AC093818.1 | PTER | APEX2 |
| LLNLF-176F2.1 | KLF8 | CYB561 | CD99P1 | CD99P1 | PLEKHF2 |
| PORCN | IGFBP6 | PTBP3 | EIF3I | AGBL3 | ADI1 |
| RP11-656D10.7 | HSD3B7 | ICT1 | LENG1 | TSPY26P | TIMM10B |
| DUSP14 | ADCY4 | CTD-3105H18.4 | CCDC150P1 | CIRBP-AS1 | SNN |
| RP11-435F17.3 | RP11-138A9.2 | AC069282.6 | SERINC1 | RP11-722E23.2 | ZRANB3 |
| ATG13 | RNASEK | LMBR1 | FAM157C | CEMP1 | IGDCC4 |
| ARHGAP8 | RP11-166B2.3 | THAP6 | NUTM2A | RTN2 | RP11-783K16.13 |
| PWAR6 | KLB | THAP4 | TVP23B | FTSJ2 | Sep-09 |
| RHPN1-AS1 | MPG | RP11-12A20.4 | SCCPDH | RP11-4B16.1 | ABHD16A |
| KB-431C1.5 | RP11-592N21.1 | QTRT1 | RP11-186B7.4 | TMEM242 | GOLGA7 |
| RP11-16F15.2 | UBE3A | RP11-288C18.1 | SOD2 | RP11-66N24.3 | ADPRHL1 |
| RP11-120K19.3 | Metazoa_SRP | RP11-93B14.6 | BOD1 | AC005606.15 | SPIDR |
| RRP7A | MRPS9 | TNKS | COPS7A | EFHD2 | AC068282.3 |
| SMG1P6 | KDM7A | NDUFA11 | PA2G4P4 | RP11-1099M24.6 | ACOT13 |
| TMUB1 | ZNF76 | ZNF214 | TOB1 | RGCC | CTD-3014M21.1 |
| EPHX1 | ACAD9 | EEF1B2P6 | RP3-461F17.3 | PIK3C2A | RBBP8 |
| RP1-100J12.1 | FAM86DP | ARL8B | RP11-844P9.2 | ATG16L2 | GLA |
| MRPS28 | RP11-575L7.8 | TMED10 | SEC11A | ABCF2 | EXPH5 |
| TMEM185A | TALDO1 | TMEM167B | RTP4 | STARD7 | RP11-697N18.4 |
| TMEM120B | CSPP1 | TAP1 | TAOK3 | CTH | PCDHGB4 |
| C9orf3 | DPY19L4 | RBMS3-AS3 | SYPL1 | VWA8 | TNNI2 |
| MTMR11 | IMP4 | DGKZP1 | SRD5A1 | NAPA | RP11-342K6.2 |
| IFNAR1 | CTDSP1 | SAMHD1 | AC009404.2 | VCL | RP11-819C21.1 |
| MAP3K6 | PPM1K | KIAA1841 | MARVELD3 | RP11-274B21.12 | RP5-1050D4.5 |
| GSN | RP13-923O23.7 | CNTD1 | RP11-449J21.3 | ORMDL1 | PPP6R2 |
| PLAG1 | CATSPERB | SNAP29 | RP11-532F12.5 | SLC31A2 | RPP25L |
| ZNF446 | FAM173A | PRMT1P1 | GPAA1 | POLB | NBEAL2 |
| RP5-821D11.7 | LRRN4CL | GRIP1 | TMBIM6 | TOB1-AS1 | TOMM40 |
| CIPC | ETNK1 | C1orf123 | ARG1 | STON1 | RP11-20B24.4 |
| PICALM | TMEM260 | PAXIP1-AS1 | RP11-21K12.3 | RN7SL268P | DLX3 |
| DOHH | AC007318.5 | PCCB | CLN5 | KDELR2 | TMED3 |
| BMPR1APS2 | C14orf119 | STAG3L2 | SPRTN | ANKRD27 | ANKS1A |
| HMGN2P46 | PIK3CD-AS2 | SEC22A | RP11-697E22.1 | ASS1P2 | GCA |
| XPC | UBE2K | PLEKHB2 | RP11-196G11.5 | RBFA | RP1-72A23.4 |
| CTC-429P9.5 | CTD-2260A17.2 | LL22NC03-2H8.5 | ZNF217 | CLK4 | RPS7P10 |
| TIPARP | BCS1L | POLR2L | DRG2 | RPL12P16 | DNAJC15 |
| SPAG1 | FBXL17 | TP53I11 | FLRT1 | RP11-574K11.32 | FBXW9 |
| RP11-600F24.7 | GOLT1B | TXLNGY | MT-ND4 | ZNF169 | RP11-75C10.6 |
| SYTL2 | ERBB3 | AC010894.3 | KRT18P63 | CRELD2 | TEN1 |
| GHRL | CXCR5 | OR7E47P | ACOT2 | RP11-661A12.12 | C11orf45 |
| CDAN1 | TUBB8P1 | PPP1R7 | PCYOX1 | CCDC64 | RSPH9 |
| SLC9A8 | H3F3B | CTC-529I10.2 | MCOLN1 | MRPL17 | ZNF266 |
| GANAB | GTF3C2-AS1 | LAMA3 | RP11-795H16.2 | UBTD1 | ZNF182 |
| NSA2 | CHRNA5 | ANKRD37 | SLC25A30 | AC074286.1 | CTD-3184A7.4 |
| SZT2 | ZBTB44 | RP11-996F15.6 | PITPNB | CYB5D2 | TLR2 |
| CALM2P2 | MTDH | REXO4 | SOS1 | GPAT4 | LAPTM4B |
| RNF39 | TM9SF4 | AP003774.1 | RPL9P7 | FARP1 | RP11-645C24.5 |
| AP000223.42 | NUAK2 | CTD-2410N18.3 | RP11-517B11.4 | C14orf1 | TULP3 |
| TMEM214 | KRTCAP3 | RIBC1 | SMO | PURA | RP11-390P24.1 |
| TRIM23 | TFEB | RP11-31F19.1 | ZNF295-AS1 | HDAC10 | EPB41L4B |
| GTPBP10 | ST13P4 | CTD-2001C12.1 | PRR22 | SNX25 | RP11-245D16.4 |
| RP11-320M2.1 | RP11-783K16.5 | MTND2P28 | RP11-1007O24.3 | RP4-682C21.2 | TRAF3IP2-AS1 |
| ZSWIM6 | C17orf97 | RP11-347C18.5 | KLRG1 | PGAM5 | RP11-230F18.5 |
| RP13-254B10.1 | TXNL4A | TANK | FOXRED1 | EPT1 | RP11-93B14.10 |
| PGAP3 | PDIA6 | PSEN2 | NRM | CTB-79E8.3 | DTYMK |
| VIMP | VPS25 | NKAPP1 | C17orf51 | LRCH4 | TPGS1 |
| SNX18P9 | SAMD13 | AC005540.3 | RP11-165E7.1 | PLD6 | LRRC37BP1 |
| CTB-26E19.1 | ZNF585A | POC1A | MED11 | AC010226.4 | WDR43 |
| CHKB-AS1 | REV1 | CRIP1 | GUSBP3 | IMPAD1 | LINC00843 |
| RB1 | CTD-2506P8.6 | VPS37C | BRIP1 | PMFBP1 | UBE2G2 |
| C19orf12 | RP11-56B16.2 | TMEM101 | IKBIP | PICK1 | ACADM |
| SNX29 | UFSP2 | DPEP2 | FBXO42 | ERMAP | NDUFAF6 |
| CCDC146 | TMEM68 | RP11-499E18.1 | TPK1 | XPA | CH17-260O16.1 |
| EMC7 | QPCTL | GMPPB | PITPNA | OLFML1 | RP1-138B7.7 |
| MME | TMED2 | RP11-632K20.7 | ATP5A1 | RP11-152N13.16 | LINC01353 |
| RP11-278C7.1 | DDOST | RP11-467P9.1 | RP11-435D7.3 | RP1-239B22.5 | CTA-29F11.1 |
| COL8A2 | CTD-2619J13.17 | CLDN20 | CTC-429P9.3 | FGFR1 | RP11-1379J22.2 |
| SENCR | MDN1 | ZNF726 | CNTNAP1 | NASP | UNC93B5 |
| TRAP1 | CTD-2574D22.2 | SHB | RP11-231C14.7 | CTA-384D8.35 | RABEPK |
| SLC25A15 | SOWAHD | PLCXD1 | PLCXD1 | TLK2 | CTD-2017D11.2 |
| RP11-332M2.1 | FAM122B | ZNF827 | RP13-104F24.2 | RP11-118K6.3 | CTD-2574D22.4 |
| RP11-530C5.4 | AC007383.3 | RP11-218L14.4 | RP11-259K5.2 | HAUS6P1 | HINT2 |
| LIAS | RMI2 | ZNF423 | ZNF69 | AC007192.6 | IGLV7-46 |
| PROX2 | RP13-216E22.4 | MRPS18A | BCAS4 | IFRD2 | NAIF1 |
| TRIM14 | KAT2A | FAS | RP11-77H9.2 | VPS52 | RBMS3 |
| RP11-632C17_A.1 | FANCM | DEPDC1-AS1 | AC012358.8 | MTFMT | COMTD1 |
| RP1-20B21.4 | RP11-757O6.6 | RARS | PPP2R5E | TDRD12 | MAP2K2 |
| TCFL5 | ISG15 | TPI1P2 | SDC3 | C6orf89 | RPA3 |
| TRIM31 | HNRNPA1P49 | DPH2 | CCNH | KLF16 | DOLK |
| GYG1 | RP4-800G7.3 | FAM73A | MTMR3 | DYNLL2 | MRPL10 |
| STX4 | FCF1P2 | ATP6V1H | ZNF836 | WRN | RP11-48B3.3 |
| MYL12BP1 | PKIA-AS1 | TMEM198 | RPL21P28 | USP15 | LLNLR-276H7.1 |
| KIAA1161 | PHF21A | MLST8 | RUVBL2 | PDZD9 | SCUBE3 |
| SLC25A36 | CCS | TLR3 | MFSD11 | RP11-983P16.4 | STRBP |
| RBM18 | CTD-2623N2.3 | SPDYE10P | MILR1 | APOL4 | NHP2 |
| AC090945.1 | NME9 | SETD7 | ADIPOR1 | NT5C3AP1 | TEX9 |
| FOXN2 | GSS | SERPINB8 | SH2B3 | RP11-353N14.2 | CCDC125 |
| TMEM128 | UBALD2 | ZDHHC4 | C12orf71 | AC002116.7 | FBXL19-AS1 |
| UBE3B | SPATC1L | CH17-431G21.1 | WDR75 | ZNF576 | RP11-226M10.3 |
| ARAF | AC097495.2 | ZNF678 | RP5-1148A21.3 | RP11-731C17.2 | APPL1 |
| MFSD5 | OXCT1-AS1 | COX16 | BOLA3-AS1 | RABL6 | NDUFAF3 |
| NPM1P9 | RBM33 | RP11-65L3.2 | RP11-411B6.6 | AGPAT5 | C16orf95 |
| ZNF530 | CTD-2006C1.13 | GIN1 | BCRP2 | XRRA1 | TBX6 |
| CTD-2017F17.2 | FAM47E | SOGA1 | TCEANC2 | ACAP2-IT1 | CTD-2012J19.1 |
| ZFY | ORAI2 | LMF1 | CHL1 | ATP5EP2 | AKT1S1 |
| MSS51 | SLC35B2 | FCF1 | ANKRD20A18P | AP1S1 | LINC00116 |
| C19orf18 | TP53TG5 | ORC5 | CEP41 | ITM2C | ZHX1-C8orf76 |
| TTYH2 | SIRT5 | FRS3 | PRR7 | C10orf54 | NSMCE1 |
| PDE9A | ZBTB10 | LINC00205 | INE1 | RP11-131M11.3 | TMEM106B |
| IRF2BPL | RP11-50B3.4 | TRAF5 | UBAC2-AS1 | AC011999.1 | NDUFB5 |
| MUL1 | ZC3H8 | NAGLU | SGTA | FILIP1 | CCDC22 |
| LINC00106 | LINC00106 | ZNF582-AS1 | COMMD8 | RP11-166P13.4 | WWP1 |
| CTD-3234P18.6 | TEX264 | RP3-337H4.9 | GHDC | SLC25A19 | LINC00337 |
| CTGLF12P | CALML4 | SAMM50 | TAOK2 | DTNB | RHOF |
| RP11-73M18.8 | CCDC117 | SP140 | BX322557.10 | SLC43A1 | MFSD12 |
| RP11-448G15.3 | HN1 | CTB-36H16.2 | NEB | CHCHD3 | ASTN2 |
| PHTF1 | PUS1 | ANKRD39 | RP11-278J6.4 | GTDC1 | FAM227B |
| FTCDNL1 | LRRC4 | INTS8 | CCDC51 | MOB3A | B4GALT5 |
| RP11-1277A3.2 | SLX4IP | PYGO1 | HIST4H4 | KIAA0895L | SUMF1 |
| BNIPL | ZNF341 | LILRA2 | PEX12 | TMEM138 | RP11-12J10.3 |
| LCTL | PRG2 | APBA3 | UGGT2 | COMT | RP11-434B12.1 |
| TAF13 | SMIM20 | TRIM47 | RP11-950C14.10 | SIVA1 | ANKRD20A1 |
| CELSR2 | FAM210A | MRPS12 | MBNL2 | NPRL2 | RP4-657D16.3 |
| RP11-93B14.9 | PSMC3IP | PABPC1L | RN7SL481P | CDKN2B | PIGQ |
| PTPN2 | VAMP8 | MBD2 | SPAG8 | DERA | SDCBP2-AS1 |
| LINC01277 | CTC-360G5.9 | RP11-110G21.1 | TOPORS-AS1 | TIGAR | TMEM184B |
| GPR107 | MT-CO1 | TRIM4 | SYNGR2 | HNRNPH1P1 | TMEM147-AS1 |
| HSPA1A | TMED1 | TM7SF2 | ZBTB41 | TMEM53 | AGRP |
| MTFR2 | STAT1 | AMY2B | SRD5A3 | C21orf59 | AC004067.5 |
| LHFPL2 | TRIM67 | LMAN2L | NUP85 | PNKD | ADORA2A-AS1 |
| NDUFV3 | ASAH1 | LARS2 | RP11-10C24.3 | ITPKA | TAS2R20 |
| HMBS | ZNF582 | C3orf33 | ALDH3B2 | SAC3D1 | DSCR3 |
| C17orf96 | PSMB8 | RP11-274B21.14 | AKAP9 | RP11-68I3.11 | CHKB |
| LGALS8 | CTD-2600H12.2 | CCDC96 | CCNC | PPP1R32 | ROCK2 |
| RP11-382A20.3 | PXMP4 | MIER2 | AP000240.9 | AK2 | ARMC10 |
| TMX2 | RP11-617F23.1 | DNAJC5B | FLOT2 | CTC-444N24.11 | RSPH3 |
| Z83851.4 | AP3M2 | RP11-370A5.1 | CAB39 | GABPB1-AS1 | HARS2 |
| MED14 | RP11-11N7.5 | FGD3 | ZFP41 | TMEM192 | ZNF852 |
| NLN | DNHD1 | SNX33 | NR6A1 | METTL8 | C19orf60 |
| FBP2 | NAPEPLD | SUV420H1 | CDH13 | LINC01004 | CTD-2547L16.1 |
| ATXN2L | RP11-73M18.6 | KLHL21 | NLRP2 | NNAT | STK19 |
| TUBG2 | TMEM237 | CD46 | GNPAT | KB-226F1.2 | STEAP4 |
| BORCS8 | CTC-338M12.4 | PEX26 | CTD-252513.6 | L3HYPDH | RP11-400F19.18 |
| STRIP2 | RPSAP12 | RP1-198K11.5 | TXN2 | C10orf128 | RP11-174G6.5 |
| RP11-552M11.8 | RP11-121C2.2 | ATP5HP4 | CPT1A | SH3BP5-AS1 | SNHG16 |
| RP11-568N6.1 | YIPF2 | TBC1D22A | TFPI2 | RP11-407N17.4 | BCO2 |
| ENTHD2 | APPBP2 | RNMTL1 | SHC4 | MIR17HG | LYRM7 |
| UCHL3 | RP11-1149O23.3 | FOPNL | C16orf13 | GNRH1 | TFDP1 |
| C12orf66 | TMPPE | RNF135 | VPS9D1-AS1 | PSMG1 | TSSK4 |
| RP11-44M6.7 | RP3-508I15.19 | RP11-345K20.2 | RP5-1187M17.10 | CTD-2650P22.2 | KXD1 |
| C12orf45 | ANKRD30BP1 | DNAJC24 | VOPP1 | ANKRD19P | PLA2G15 |
| C15orf56 | ZCWPW2 | ASIC1 | CEL | DLST | TRAPPC2L |
| NT5C1B | NEDD8-MDP1 | DOC2A | PTPN6 | TMEM156 | IDI2-AS1 |
| RP11-442N24_B.1 | SGK494 | CYB561D2 | MYCBP | RP11-655M14.13 | NEU1 |
| CARD8 | RMND5B | HLA-F-AS1 | RBFOX2 | RNF167 | TMEM86B |
| LRRC37A3 | RP11-12M9.3 | BET1L | MRPL45 | SPR | TRPV1 |
| NAT9 | POR | RP11-468E2.11 | SLC3A1 | ZDHHC7 | RCE1 |
| TMEM150A | RP4-614O4.13 | SLC8B1 | MSRB1 | RP11-80H18.3 | USP35 |
| ZNF813 | RP11-73M18.2 | ZNF260 | RP11-379F4.7 | RP11-192M23.1 | RPL4P6 |
| UQCC2 | RP11-705C15.3 | MFSD1 | HIVEP1 | DOCK9-AS2 | ZBED6CL |
| NEDD4L | EIF2S1 | TRAF1 | CES3 | RP11-420K14.6 | CSTF3-AS1 |
| AC021188.4 | CTC-471J1.8 | RP11-304L19.12 | RP11-817O13.8 | C22orf46 | NSUN2 |
| ZNF383 | SLC30A6 | HDAC3 | UBL7-AS1 | LINC00920 | CECR7 |
| RP11-563J2.3 | RP11-90L1.8 | H2AFJ | APOOL | RP11-7F18.2 | PAPD5 |
| SLFNL1-AS1 | RGL2 | CAD | ACVR1B | BIN3 | YIPF4 |
| GINM1 | SLC35F6 | KIAA0753 | RP11-1020A11.2 | RP11-712L6.5 | ZCWPW1 |
| RP13-514E23.2 | SLC25A26 | AHSA2 | SLC16A14 | RP11-177B4.1 | BBC3 |
| RP11-284B18.3 | KRBA2 | ASL | RP3-405J10.3 | RP11-141B14.1 | CDK4 |
| BECN1 | SWI5 | ICAM4 | ALG10B | SLC27A4 | FGFR4 |
| SLC51A | RP4-798A10.2 | NAA35 | SNRNP27 | CCZ1B | RP11-697N18.3 |
| ABHD10 | PCDHGB5 | CA9 | IFT74 | CIB1 | COX14 |
| TATDN2P2 | LMNA | PCDHGB3 | RP11-546D6.3 | LDHAL6B | AK5 |
| RP11-79P5.10 | PKIB | UIMC1 | SP140L | WEE2-AS1 | RP11-48B3.5 |
| RABGGTA | CTD-253719.13 | CTC-241N9.1 | SNHG3 | MRPL30 | PGAP2 |
| ANGEL1 | MEMO1P1 | IL17RC | FOS | CLCN4 | ZBTB45 |
| NFIA | SP100 | MBD5 | TMEM97 | CLDN15 | PPFIA1 |
| RP11-106M3.2 | RP11-196G11.4 | TAF9 | GALE | C1GALT1 | CHCHD5 |
| HCCS | LINC01572 | TRAPPC9 | ZNF26 | C15orf65 | BCKDHA |
| CTD-2410N18.4 | PRDX2 | STARD4 | IPO7P2 | RP11-523O18.5 | CELSR3 |
| TUBGCP4 | PUS7 | CSTB | GNL3LP1 | RP11-360L9.4 | RP11-407N17.5 |
| ORAI3 | RAD54L | LRP4-AS1 | MMP25-AS1 | NPIPB11 | MTTP |
| U2AF1 | TXNDC5 | C2orf16 | RP11-299J3.8 | PAFAH1B2 | MDH2 |
| NT5C3A | SWSAP1 | ERICD | RP5-1039K5.12 | NCKAP5L | HEXA |
| CFAP36 | RP11-16E23.5 | CREBZF | RP11-211N8.2 | RP11-373L24.1 | ITCH |
| TRAF7 | HACD2 | SLC12A5 | GSTK1 | AC024592.12 | PSMD6-AS2 |
| FSBP | IGLV4-69 | PHBP5 | SNX17 | GSTP1 | CLASP1 |
| PLCXD2 | FAM159A | TMEM41B | ZNF655 | ST8SIA4 | LINC00501 |
| PINX1 | IFNGR1 | VDAC1P1 | OARD1 | ACTG1P3 | RTN4IP1 |
| RP11-355B11.2 | RP11-215P8.3 | MANBA | AKR7A2 | ZNF616 | SCARB2 |
| TRIT1 | LY6E | AC092171.4 | AC083899.3 | SLC16A1 | RP11-6N17.6 |
| CCDC47 | RP11-297B17.3 | SCN8A | CTC-250I14.3 | MOSPD2 | MYO1D |
| VAMP3 | TTC13 | ELF3 | MOV10 | VSTM5 | NTMT1 |
| THEM6 | ZMYND10 | FAM131A | CYB561D2 | HSD17B6 | UNC119B |
| SETD3 | CPNE5 | JAK2 | TNFRSF10D | SCAI | PTCD2 |
| TM2D2 | CTD-2528L19.6 | EMG1 | LINC00562 | PANX2 | FAM71F2 |
| IDH3A | GATAD1 | UBE2D2 | PALD1 | MTMR10 | RP11-85I17.2 |
| RP11-517P14.2 | LRRC61 | CPNE3 | ALKBH4 | RP11-667M19.9 | MOV10L1 |
| MTFR1 | RP4-657E11.10 | LINC00094 | PTGR2 | ZBTB11 | PINLYP |
| SETMAR | CTD-2083E4.5 | SETD4 | RP11-497H17.1 | CLK3 | PIGW |
| TAC01 | RNF130 | SLC37A1 | SHANK2 | C8orf44-SGK3 | NOXRED1 |
| SSR1 | PIGO | STX1A | LYRM2 | FAM227A | RP11-151A6.4 |
| RP11-402J7.2 | C1orf61 | GM2A | NDUFAF4 | CTD-2308B18.4 | TBC1D2 |
| VTI1B | UBN2 | CCNE2 | COG5 | CTD-2521M24.5 | EXD2 |
| HHLA3 | CTD-2521M24.4 | CFAP206 | FBXW11P1 | TAZ | MALAT1 |
| CTD-2095E4.5 | RNASEH2A | PCDHGA8 | RP11-1094M14.11 | VDAC1 | TBCD |
| IKBKB | PTDSS2 | CTC-265F19.1 | RP11-637A17.2 | RP1-12G14.7 | MRPS25 |
| APOL5 | AL136419.6 | LRRC23 | PLGRKT | BZW1P2 | MRPL37 |
| NOMO2 | NCOA1 | ZNF551 | PIGU | HNRNPKP1 | IFI44L |
| HSDL2 | ISCA2 | LCOR | TFCP2L1 | RP11-155G14.5 | AC007285.6 |
| SLC2OA2 | RPN1 | METTL16 | RP11-334G22.1 | RP11-274B21.3 | MRPL19 |
| LPIN3 | LA16c-358B7.3 | ENTPD5 | LRRC37A15P | DCAF10 | DDX60L |
| ABHD2 | ITSN1 | DGAT1 | RCN1 | SMC1B | UBE2R2 |
| AC109826.1 | CUL9 | RP13-131K19.2 | SLC31A1 | CTC-454I21.3 | BDH1 |
| RTN4 | OXNAD1 | ATP5SL | RP13-516M14.4 | RP4-635E18.9 | LSM12P1 |
| TIAF1 | MT-CO3 | RP11-977G19.10 | RDH14 | RP13-188A5.1 | CD302 |
| SGK3 | BBOX1 | SMTN | SPECC1L-ADORA2A | RP11-167P11.2 | GPM6B |
| LINC01140 | DBT | CCDC120 | RP11-117D22.2 | VPS39 | PAK6 |
| RPL23AP7 | SMIM11A | ZC3H12D | STRA13 | RP11-134E15.2 | C8orf33 |
| NONOP2 | COQ6 | RPL26L1 | RP11-757O6.1 | FAM95B1 | FAM169A |
| TICRR | MRPL21 | HIATL1 | HSD17B7P2 | KCTD5 | PAM16 |
| CTC-479C5.10 | SCAMP2 | RP11-182L21.5 | SLC24A1 | RNF44 | ZFP36 |
| VPS13C | LARGE | MAPK8IP2 | BID | RP3-426I6.6 | SLC35A2 |
| RP11-527J8.1 | RP11-74J13.8 | CNR2 | TTC39A | CREBRF | RP11-205M5.3 |
| RP11-231E4.5 | BCL2L13 | NAA10 | EMC6 | CHST7 | C1orf210 |
| TUBG1P | ENOSF1 | C1orf109 | RP11-1H8.5 | RP13-392I16.1 | MTMR9LP |
| MCEE | MAP3K13 | AC018804.7 | ALG2 | RP11-70L8.4 | ZNF821 |
| PLOD1 | DDRGK1 | RP11-521B24.4 | FMO4 | PDCD5 | CTD-2616J11.10 |
| STAG3L3 | ACADS | RP11-809N8.2 | PIK3IP1 | NFKBIB | BNIP3P11 |
| LMAN1 | ZNF785 | APOL2 | CABP4 | RP11-87C12.2 | BLCAP |
| TMEM33 | KB-1125A3.12 | C1orf159 | GCNT2 | PCDHGA6 | IQCC |
| ZNF600 | CARS2 | TMEM141 | KLF13 | HSF2BP | CCNT2 |
| TMEM256-PLSCR3 | SLC25A41 | GRK6 | C2orf47 | MRPS2 | LAMP2 |
| CYP3A5 | RP11-104O19.4 | AP001055.6 | APOBEC3B | COCH | KSR1 |
| TMX1 | SLC25A42 | DNAJC18 | BROX | RP11-497G19.1 | AC008697.1 |
| SLC25A35 | FADD | PRPF40A | SLC1A5 | LIN52 | FAM83H |
| GDF9 | ERVW-1 | EBNA1BP2 | CTB-43E15.2 | SEC63 | FBXL4 |
| ATP2A1-AS1 | MTAP | USP34 | CLN8 | RP11-247L20.3 | C20orf203 |
| ECHDC2 | RP11-861A13.3 | PROB1 | CLIC2 | RP11-1148L6.8 | RP11-467L13.5 |
| RP5-1057I20.2 | PANO1 | RP11-379K17.2 | SEPT7-AS1 | SEC22C | FHIT |
| NCK1-AS1 | AC046143.3 | HAS3 | MLLT10 | RP11-45P15.4 | CTC-550B14.7 |
| CHEK2 | AC009133.21 | PTRH1 | SLC37A4 | RTEL1-TNFRSF6B | GALT |
| HIBADH | TMEM107 | MCMDC2 | DDX11-AS1 | TCTA | TCL6 |
| SLC27A5 | HEXB | RP11-381K20.2 | TEAD2 | LINC01355 | NME6 |
| VDR | FBRSL1 | GRIK5 | ZNF599 | MON1A | TRIM39 |
| RP11-61L23.2 | RP11-255C15.3 | CLPB | INPP5E | STK3 | C19orf38 |
| MUC6 | GPR176 | EMC8 | NANS | ST20 | SCARNA15 |
| SKP1P1 | RP11-46D6.1 | AIF1L | P2RY11 | DNAJC30 | FLJ42969 |
| YIF1B | NAA50 | GGTA2P | BCAS1 | SNX21 | CD19 |
| ABCB10 | ZSWIM4 | AP000289.6 | ANKDD1A | HSCB | PTPRD |
| CTD-3216D2.5 | SEPW1 | TTYH1 | MMP23B | UBA52P6 | ABCD4 |
| ZFAND4 | GRAPL | EIF3K | EMB | NRG4 | RP11-488C13.5 |
| RP11-5C23.1 | RP11-613D13.5 | P2RX1 | FAM117B | MTCO1P12 | C20orf96 |
| RP11-2B6.2 | LYL1 | C8orf58 | RP11-324O2.3 | RP4-647C14.3 | ZNF611 |
| CACNA2D4 | PI4KAP2 | SKOR1 | AC016747.3 | APOM | RP11-574K11.29 |
| RP11-536K7.3 | LMTK2 | ABCB9 | RP11-497H16.9 | IMPG2 | RRP8 |
| DERL1 | NSUN6 | FRZB | ASMTL-AS1 | ASMTL-AS1 | SLC25A16 |
| TSPAN33 | LRIT3 | TET1 | RP11-514P8.6 | C1orf147 | TAMM41 |
| GPT | RNF138 | PWP2 | CBWD3 | AC004980.7 | RP11-77E14.2 |
| CYC1 | GRK4 | AC091729.9 | PUM3 | RELT | ZFAND6 |
| SLFN13 | ENTPD3-AS1 | SLC39A4 | OTP | TAGLN | LACE1 |
| PRR7-AS1 | LINC00852 | TFR2 | ZDHHC12 | NAA16 | USF3 |
| CTSO | NOCT | TFAM | PLPP6 | SPCS2 | ABHD8 |
| RP11-585P4.5 | LAMTOR4 | TRMU | CTD-2292M16.8 | CTD-2544N14.3 | AGPAT3 |
| RP11-258C19.7 | RP11-345P4.7 | MINCR | CTD-2353F22.1 | NRSN2 | RAP1B |
| LRRFIP1P1 | SH3GL1P1 | C15orf39 | TROVE2 | RP11-383F6.1 | DNASE1L2 |
| LINC00471 | MAP3K8 | YJEFN3 | RAP1GAP2 | RP1-59D14.5 | PRIM1 |
| DUS4L | RP11-426A6.5 | TLN2 | PUS10 | MTIF2 | MAML2 |
| SH2B1 | MFI2 | POLR1C | BRCC3 | RP11-271K11.5 | SCO1 |
| GPR82 | SMAD6 | TMED9 | VASP | LA16c-321D4.2 | IFT81 |
| MT-CO2 | RPL24P2 | TRMT44 | NPM1P24 | AC034220.3 | RAPH1 |
| FAM109B | SNRNP48 | CTC-471J1.11 | LINC00893 | CCER2 | LAMP3 |
| SDCCAG3 | DNAH3 | PRR13 | RP4-734P14.4 | HLA-DRA | PNPT1 |
| GFM2 | BORA | RP11-582J16.5 | APOL3 | ATG14 | PLSCR1 |
| PRKCSH | NAPB | PSMA8 | TIMMDC1 | LRRFIP1 | BORCS8-MEF2B |
| RP11-723O4.2 | PREPL | FUK | RDH11 | NPEPL1 | CD164 |
| RP11-138I18.2 | CYGB | FASN | HEXA-AS1 | GFM1 | MZF1 |
| TAF6L | RP11-676J12.4 | CASZ1 | NEIL2 | ZNF563 | NDUFA8 |
| RP11-134G8.5 | C12orf77 | PIGF | CTA-292E10.8 | LINC01521 | CTB-131B5.5 |
| NUDT14 | BAX | ALG5 | SUB1 | CMPK2 | AC005083.1 |
| CCDC64B | AF131216.6 | ACO2 | LINC00654 | PARP12 | LA16c-390E6.4 |
| TFB1M | NEIL1 | SWAP70 | FAM118A | MB21D1 | RP1-266L20.9 |
| RP11-291L22.9 | TMEM175 | MTFP1 | RPS6KB2 | TNFSF9 | PARD6B |
| KCNK6 | GLCE | CDKL1 | KCNAB1 | AC142528.1 | APOBR |
| RP11-563J2.2 | LINC00643 | RP11-685N10.1 | NCF1C | VAC14-AS1 | RP11-415F23.3 |
| MCU | SERPINF1 | DDX11L2 | RWDD2B | CTD-2369P2.10 | GPR68 |
| SUSD3 | LRRC37A2 | LPCAT3 | JAM3 | CTA-313A17.3 | RP11-186N15.3 |
| PHF7 | ARMC7 | ZNF487 | RP11-727F15.12 | KRT18P59 | ZNF18 |
| TAS2R64P | MFSD9 | SREK1 | TFE3 | DPH1 | RIMKLBP1 |
| STARD7-AS1 | RP11-802O23.3 | MIRLET7BHG | LINC00449 | CKB | RP11-16E18.3 |
| SDCBP2 | RP11-700J17.1 | CCT6B | RP11-21J18.1 | SAT1 | C17orf58 |
| RP11-770J1.5 | RNF121 | MIR4435-2HG | RARG | RP11-538I12.2 | RP5-966M1.6 |
| PIGG | AFMID | ATP5S | TTC28 | MAP7 | B4GALT7 |
| KDF1 | CTD-2228K2.7 | ICAM5 | CD63 | TTC26 | RP5-827C21.4 |
| TM9SF2 | N4BP3 | SFXN4 | CXXC4 | BNIP3P18 | AK1 |
| AC002116.8 | ZNF132 | NBR2 | B4GALT1 | GPR157 | TECRP1 |
| DHRS11 | GALNT6 | LGR4 | LRRC56 | LRPPRC | AMFR |
| LINC01232 | HSPB1 | RP4-563E14.1 | NDC1 | MYLIP | CBSL |
| FAM53B | EBP | SLC4A7 | MYO9A | RP11-752G15.8 | CDCA4P1 |
| CTD-2196E14.6 | ZMAT2 | RP11-10A14.4 | IFT88 | ARHGAP27 | FSTL3 |
| RCN1P2 | ITSN2 | CTD-3162L10.1 | RHOV | GHRLOS | RP3-323A16.1 |
| AKIP1 | LCT | RAB12 | GLB1L | RP11-347C12.3 | ZKSCAN2 |
| GCNT1 | MLLT3 | CARKD | RP11-677I18.3 | RRN3P1 | PISD |
| RP1-92O14.3 | MTCH2 | RP11-1036E20.9 | TXNRD2 | TP53INP2 | RP11-503E24.2 |
| NFU1 | CTB-92J24.3 | CDPF1 | FOCAD | MXD3 | PARVB |
| STPG1 | TSPAN17 | CTD-2240E14.4 | LY6G5C | ATP6V0D1 | CASP10 |
| RP11-674N23.4 | RP11-486O12.2 | RASL10A | RCL1 | MGAT3-AS1 | DDHD2 |
| ARFGEF1 | CLCN7 | DUSP7 | LLNLF-173C4.1 | ZNF23 | ZNF34 |
| CHKB-CPT1B | CCR6 | SLC38A6 | RP11-97O12.6 | NEAT1 | ERLIN2 |
| PAPLN | LRPAP1 | MTHFR | EME1 | ACAP3 | SDHAF3 |
| RP11-521I2.3 | TCP11L2 | PI4KAP1 | CTD-2396E7.11 | ALDOC | C19orf24 |
| TP53I13 | LINC00926 | RP11-4O1.2 | SV2B | FURIN | AIFM2 |
| MRPS15 | CDKN2B-AS1 | CTD-2540L5.6 | C1GALT1C1 | N6AMT1 | TPPP |
| ZNF224 | RP3-388M5.9 | AD000671.6 | CMC4 | C2orf74 | KDELR1 |
| RP11-50E11.2 | LINC00216 | CD180 | CAPG | GRIN2D | ISG20 |
| AC006014.8 | CTNND1 | RP11-802E16.3 | ALKBH6 | SLC39A3 | C19orf71 |
| MPEG1 | RP5-1120P11.1 | BMP1 | SPINT1 | PMAIP1 | SPNS2 |
| TCF20 | RP11-104N10.2 | ZNF585B | AC099850.1 | RP11-278C7.4 | ALKBH7 |
| SRP19 | RP11-326A19.5 | PCMTD1 | ZNF561 | TMEM57 | CYP51A1 |
| AC012146.7 | RP11-640L9.2 | RP11-180C16.1 | HMGN5 | COLEC11 | TMEM99 |
| GATB | MYBPC1 | ALG10 | RSG1 | ALOX12-AS1 | AC114271.2 |
| ZNHIT1 | RIBC2 | MAP6D1 | TKT | CLIP1 | LPAR2 |
| TBC1D3C | BCAP31 | RP11-415F23.2 | SDHD | LIME1 | GALNT12 |
| TMEM181 | TFB2M | PBX4 | IFT22 | XXbac- | GRAMD1C |
| | | | | B461K10.4 | |
| RP11-274B21.4 | WDFY4 | FBXW4P1 | SNX16 | GPR137B | HERC2P3 |
| TTF2 | COX18 | RP11-488L18.10 | RP11-490O6.2 | LBX2-AS1 | SLC25A34 |
| YRDC | DSTNP2 | ZZZ3 | RP11-96D1.8 | ZBED5-AS1 | SSH1 |
| SYNE2 | RP11-48B3.4 | ANKRD18EP | LTB4R2 | RP11-130L8.3 | MNT |
| ZNF619 | CCDC183-AS1 | ZDHHC18 | C9orf43 | NDUFAF1 | HAUS2 |
| DHCR24 | GSE1 | AQP11 | MYO6 | AC007880.1 | SLC29A1 |
| CRCP | XX- | WDR97 | STARD4-AS1 | PQLC3 | METTL15 |
| | C00717C00720L.1 | | | | |
| YTHDF3-AS1 | CCBL1 | RP11-206L10.9 | BST2 | WASH7P | CCDC134 |
| MIB2 | RP11-418J17.1 | NRBP2 | KLHDC1 | FBXO44 | FADS1 |
| CTA-217C2.2 | XXbac- | UBE2D4 | ACSL3 | KCTD21-AS1 | TBL2 |
| | BPG32J3.22 | | | | |
| OXSM | HMGCS1 | RP11-798G7.8 | HMGA1P4 | PLBD2 | CTPS2 |
| RP11-553K8.2 | RP11-274B21.2 | SLX1A-SULT1A3 | CCDC137 | SLC25A11 | SLC35F5 |
| ZNF860 | RP11-482G13.1 | MFSD4 | KIN | MRPS7 | TPD52L2 |
| NFIA-AS1 | TPT1-AS1 | DBNDD1 | MINK1 | AGAP5 | RP3-368A4.5 |
| DNAJB14 | NIPAL3 | CXCL16 | MMP11 | PPM1M | DEGS1 |
| RP11-97C16.1 | XBP1P1 | RP6-65G23.3 | RNF19B | WDR45 | ASCC1 |
| MED18 | TMEM44-AS1 | CTD-2186M15.3 | RP3-486I3.4 | LLNLR-307A6.1 | TMEM87B |
| P4HA1 | SPON2 | CCNG1 | ZDHHC21 | AGGF1P1 | KMT2E-AS1 |
| RP11-500C12.1 | RP11-386G11.10 | HEXIM1 | HSP90AB3P | KIAA1549L | ITM2B |
| NPHP4 | ADCK4 | NUCB1 | ALMS1-IT1 | ARHGAP42 | POFUT2 |
| MSR1 | FAAP24 | FAM96A | CTD-3224K15.3 | AKR7L | NOP16 |
| COLCA2 | IGLL5 | RDH13 | ZNF32 | SEC24B-AS1 | ZNF573 |
| KIFC2 | CSF2RB | NSUN5P1 | RP11-689B22.2 | SLC9A3R1 | CAAP1 |
| MSMO1 | FLVCR1 | ACP5 | CTD-2012K14.6 | GLIDR | DLX1 |
| ZNRF3 | ARHGAP19-SLIT1 | C1QL3 | FAHD2CP | RP3-468K18.6 | NCBP3 |
| DOCK8 | C14orf142 | LTBP2 | INCA1 | MXD1 | RP11-498C9.3 |
| MED28 | NABP1 | RP11-326C3.12 | STYX | FAM156A | CTA-276F8.1 |
| RP11-73M18.10 | AC002310.7 | ENDOV | CTD-2587H24.14 | GPR18 | SLC35A3 |
| TM2D1 | IFNW1 | AC002310.12 | ADGRE5 | SLC35E3 | RP4-756G23.5 |
| PALM2-AKAP2 | MBTPS2 | ITGAV | GTF2IRD2P1 | TPP1 | SYTL3 |
| RP11-372K14.2 | CTC-203F4.2 | MRPS18B | CTC-487M23.8 | MRPL28 | RP11-134G8.7 |
| BSG | LZTFL1 | PIGL | NDUFV1 | ZMIZ2 | ITGA6 |
| AF127936.5 | SLC43A3 | HRK | RDM1 | RP11-930P14.2 | AC127904.2 |
| PRR15L | MAN2C1 | ERMP1 | AC145343.2 | PPT2-EGFL8 | NPR2 |
| PM20D2 | Z97634.3 | SPDYE6 | ATP11A | MAGIX | C9orf9 |
| ANKRD9 | ORAI1 | HELLPAR | TST | HVCN1 | LRTOMT |
| RP13-638C3.2 | HSD17B12 | NAGA | RP11-888D10.4 | SLC22A31 | WDR4 |
| EIF4EBP3 | CTB-89H12.4 | CYP4F35P | LL0XNC01-116E7.1 | GOLM1 | ST7L |
| ANKRD20A4 | NIFK-AS1 | NAAA | ARRB2 | SERPINI1 | LRRC20 |
| RP1-197B17.7 | RC3H2 | HDDC3 | TBX10 | SLC6A4 | PRR34-AS1 |
| FBLN7 | DISP2 | UCP2 | AP000432.1 | RP11-33O4.2 | MBOAT7 |
| ANKRD42 | NPC1 | CTB-5E10.3 | DAPK3 | RP11-529K1.3 | PTPN7 |
| EME2 | REEP4 | RP11-496H1.1 | RPS15AP1 | ARRDC3-AS1 | PLPP5 |
| TSPAN13 | TMC6 | KLHDC7B | OCLM | MECP2 | RASA4CP |
| NOMO1 | FAM212A | RP11-23N2.4 | LDLR | PRICKLE4 | NCKAP1 |
| RP11-356M20.1 | PAN2 | TMEM201 | CYP51A1P2 | DLL1 | FAR2P1 |
| C11orf53 | COPRS | RP11-143K11.7 | RP11-141O15.1 | CTD-2574D22.3 | TMEM161A |
| TMBIM1 | EFEMP2 | RP11-576C2.1 | CNN2P9 | RP11-513M16.8 | ICAM2 |
| BBS4 | TBKBP1 | TXNDC15 | RP5-1092A3.4 | DESI1 | ELOVL6 |
| PHPT1 | SIRT6 | TCEB1P19 | RP11-686F15.2 | L2HGDH | MTND5P11 |
| STK11IP | RP11-34P13.13 | ELOVL1 | RP11-295D4.1 | RPL12P25 | LINC00339 |
| FAM129C | IQCE | LAPTM5 | CRB3 | ATP9B | SIDT2 |
| DNASE2 | CACNA1C-AS1 | HSD17B8 | PACRGL | PHYKPL | ANGEL2 |
| CYBA | DNAJB6 | NCF1 | SPATA12 | TDRD6 | BCLAF1P2 |
| RGS14 | IGLV7-43 | PCDHGA4 | DCXR | ZFP14 | RP11-342I1.2 |
| PIK3R2 | L3MBTL4 | TMEM161B | ZNF613 | RAB11FIP4 | RASA3 |
| TYRO3 | IFT140 | METTL1 | AGER | MRI1 | MACROD1 |
| D2HGDH | ZNF692 | BZRAP1 | ARID3B | RHPN2 | MRPL2 |
| NUDT19 | PDXDC1 | PIEZO1 | PARP16 | NMI | RABL2B |
| FBXL2 | RP11-146D12.2 | FAM86C1 | PTCD3 | RP11-296I10.3 | UBR5-AS1 |
| GRHPR | PILRA | ZNF440 | FBXL22 | SNAI3 | NUDCD2 |
| CXorf38 | CD72 | STX10 | FAM160A2 | LRP1 | VASH1 |
| APOPT1 | GP1BA | FLYWCH1 | AGR2 | ANTXR2 | SMDT1 |
| PRR33 | AC017104.6 | RP11-381O7.3 | GMPPA | NFAT5 | RP6-159A1.4 |
| RAB40B | ARHGEF33 | AP006222.2 | RP11-127I20.8 | FAM76B | MAPK12 |
| APOL6 | MYO5C | FASTKD1 | POLR2J3 | AC024560.2 | MTHFS |
| CKLF | WDR66 | PELI3 | RP1-179N16.3 | EHD4 | TYMSOS |
| CRYBA4 | CEP19 | RP11-809O17.1 | SLC22A18 | BET1 | LRRC34 |
| SSTR2 | ZNF572 | DNAH17 | UNKL | NDUFB2-AS1 | ARL2 |
| C8orf37 | TNRC6C-AS1 | ATP6V1C2 | BTBD6P1 | LINC01547 | AP000347.2 |
| MROH1 | SORBS3 | PSMG4 | ANKRD23 | SATB1 | RP11-33B1.4 |
| PDCD6IPP1 | AC003092.1 | TP63 | ALG3 | CCDC38 | RP11-161M6.2 |
| SLC12A9 | MIPEP | RP11-111F5.5 | ZBTB21 | ENDOD1 | DNAAF3 |
| SCAMP5 | IQCG | CTD-2538C1.2 | ARSG | EPB41L1 | ABCB6 |
| PPCDC | TRIM16L | HAGHL | ABCA1 | LRIG2 | CYP4V2 |
| MST1L | RP11-480A16.1 | NDFIP2 | ANKRD26 | LRRC43 | DLEU2L |
| RP11-529K1.2 | NDST1 | JSRP1 | GLB1 | CCDC30 | OXA1L |
| DYX1C1-CCPG1 | CABYR | RP5-1074L1.4 | HOMEZ | NR1H3 | CBR1 |
| TMEM174 | CMTM4 | EIF1AD | C6orf136 | DRC3 | ZNF717 |
| RAB11FIP1P1 | TBC1D2B | PDHX | PPP1R21 | SLC16A13 | CTD-3094K11.1 |
| CMB9-22P13.1 | SDK1 | GOLGA8H | RP11-866E20.3 | DCAKD | RP11-486B10.4 |
| RETSAT | AC013461.1 | FAM86B3P | FAM132B | EPB41L4A | TLCD1 |
| CTA-246H3.12 | LRFN1 | CAMP | REC8 | SETD9 | NIPSNAP3B |
| FECH | FAM21FP | ODF3B | TTN | SDHA | CTB-147N14.6 |
| CRYZP1 | RBM6 | LGALS3BP | GMDS-AS1 | MAN1B1-AS1 | ADAM8 |
| RP4-800G7.2 | C2CD2 | CH17-340M24.3 | ABHD12 | XPNPEP3 | CD40 |
| CTA-292E10.6 | PIGBOS1 | SLC9A7 | CH507-9B2.3 | SPRYD3 | INSIG1 |
| GS1-124K5.2 | IL9RP3 | SMIM10 | RP5-967N21.11 | CCDC87 | FAM114A1 |
| IL18BP | C17orf104 | FKBP11 | RP4-758J18.13 | TRPV2 | DHRS1 |
| PCOLCE | RP11-517H2.6 | ADAP2 | CTSA | TNK2-AS1 | SHPK |
| SPN | ACAD11 | DLGAP4-AS1 | DNAJC17 | NFYAP1 | JMY |
| HMGB3P4 | FRMD4A | PDSS2 | TXNDC11 | CYP27B1 | AC005944.2 |
| CLN3 | PPM1F | SPATA33 | AC006486.10 | RP11-577H5.1 | ARHGAP20 |
| RP11-657O9.1 | STAG3L5P | ANK3 | SLC2A11 | ATP13A1 | SACS |
| RP11-613M10.8 | C1S | MOB3C | SH3YL1 | AC005532.5 | RP11-343C2.11 |
| STIM1 | ATP6V0A1 | ZNF181 | CTB-52I2.4 | TMEM199 | FAM160B2 |
| RP11-234B24.4 | LAT2 | RP11-566K19.6 | MX1 | SLC25A40 | LPCAT1 |
| SCD | CHRNA6 | ATP5J2-PTCD1 | S1PR4 | CORO1B | TK1 |
| TSPAN3 | PPP2R5A | TCTN2 | APOL1 | CYTH1 | NADK2 |
| RP4-591C20.9 | GANC | RP13-516M14.1 | NPTX1 | CLCN6 | RP1-60O19.2 |
| GNPDA1 | CTD-3105H18.14 | CLDN7 | MAG | SCARNA12 | NIPAL2 |
| LRRC39 | CSRP1 | DMD | HSBP1 | TCAF1 | AC062029.1 |
| SLC25A33 | RP11-291B21.2 | CTD-2012J19.3 | TRIM73 | TRIM66 | POMT1 |
| BBOF1 | SHTN1 | TXLNB | PTGS1 | PCBP1-AS1 | ACPP |
| SULT1A4 | GDPGP1 | SLC16A1-AS1 | RP4-694A7.2 | ACER3 | OAS3 |
| SLC16A3 | LA16c-306A4.2 | CRTC1 | MEIG1 | UBAC2 | MLANA |
| ANKRD7 | ABCC10 | SBF2-AS1 | BLVRA | NOTCH1 | ZNF658 |
| RP11-930P14.1 | CBS | CENPP | RP11-848P1.2 | NPB | MRPL40 |
| EXD3 | GUCA1B | DHX58 | CRTAP | PQLC2 | FOXH1 |
| EPB41L5 | NDUFA10 | AC006547.8 | WI2-87327B8.2 | PDXK | TARBP1 |
| CGREF1 | UAP1L1 | LINC01006 | ZNF660 | RP11-309L24.10 | CICP27 |
| ESPNL | WDR90 | RP11-552F3.12 | ZNF701 | PDDC1 | KIF9-AS1 |
| TJP2 | ADORA2A | SENP8 | YIPF6 | SPNS1 | CTD-2026D20.3 |
| ZNF485 | ZFP90 | ITGB4 | RP11-513M16.7 | FDXR | TYMP |
| KLRAP1 | RP11-521B24.3 | CITF22-1A6.3 | SLX1B-SULT1A4 | BISPR | C3orf35 |
| TNFSF15 | METAP1D | CTD-2083E4.7 | B4GAT1 | MTFR1L | LINC01222 |
| FRG2C | WASIR2 | STK36 | RP11-394B2.5 | HMGCL | RNF149 |
| RP5-832C2.5 | DCTD | RP11-797A18.5 | CNTNAP2 | ACOT1 | HLA-E |
| RP11-111F5.4 | HYAL3 | AC005076.5 | PCYOX1L | LCAT | RAB29 |
| UMAD1 | LIPJ | SPTLC2 | ADK | CRYM-AS1 | TSPAN31 |
| FOXRED2 | PODXL | DND1P1 | RP5-1142A6.10 | IQCH | PON2 |
| RGPD5 | TSTD3 | RP11-574K11.24 | RP11-124D2.7 | ADAM15 | ZNF460 |
| RP11-504P24.3 | RP11-424G14.1 | GALK1 | NUMBL | ACACA | NEK8 |
| HACD4 | C16orf86 | TMEM209 | XXbac- | POMT2 | CAPN12 |
| | | | BPG181B23.7 | | |
| PTK6 | PNPLA4 | PRMT5-AS1 | BBX | EPS8L2 | VSIG10 |
| PVT1 | RND1 | ZBTB46 | NCS1 | CTD-2366F13.1 | MANEAL |
| RP11-31H5.3 | CTC-281F24.5 | WWOX | PLCG1 | SERTAD2 | RP11-864I4.1 |
| DUSP3 | RP11-574K11.31 | PLOD3 | RP11-20G6.3 | OR7E126P | MAP7D2 |
| SEC14L1 | HDAC11 | P4HTM | RP11-471B22.3 | LINC00877 | PCBD1 |
| ELFN1-AS1 | RASA4B | XXbac- | NHLRC3 | TMC8 | B9D1 |
| | | BPG181M17.6 | | | |
| CAMKK1 | MKLN1-AS | TMCO4 | FAM9B | RPS6KB1 | RP11-362K14.7 |
| LTB4R | STAG3L5P-PVRIG2P- | AC011747.3 | IPCEF1 | DARS-AS1 | RP11-311C24.1 |
| | PILRB | | | | |
| DISC1 | HNRNPU-AS1 | AC156455.1 | ARMC9 | RP11-13P5.1 | GPER1 |
| PXN | RDH5 | LETMD1 | RFT1 | SMPDL3B | PDLIM5 |
| WDR73 | RP11-53O19.3 | PLS1 | RNPEP | RP11-166O4.6 | CTC-425O23.5 |
| CEBPD | RP11-228B15.4 | PGPEP1 | ABHD11 | CTD-2587M2.1 | Sep-11 |
| GATS | SCAMP4 | ULK4P1 | ORAOV1 | DNAH8 | TRIM65 |
| RP11-932O9.10 | IVD | CCDC57 | CATSPER2 | SREBF1 | PLLP |
| LINC00467 | RASL10B | RP4-669L17.10 | RP11-98I9.4 | CTA-941F9.10 | RP11-458F8.4 |
| PMEL | RILPL2 | KIAA1033 | NBL1 | CD37 | H6PD |
| UNC93B1 | C17orf99 | ZNF789 | DEGS2 | C12orf75 | CRIP3 |
| ANKRD10-IT1 | AC008079.10 | SEPN1 | TGIF2-C20orf24 | RRAGB | UBA7 |
| PYCRL | THYN1 | RP5-965G21.4 | CELSR1 | AC025171.1 | DMPK |
| PILRB | SLC45A3 | LINC01347 | SLC29A2 | LCMT1-AS2 | RPL23AP82 |
| CD27 | RP4-583P15.15 | CPEB4 | ACOT4 | TMEM91 | CASKIN2 |
| ETFB | CICP3 | TMEM198B | RAB36 | SLC17A9 | MOB1B |
| FARP2 | GNGT2 | IGHV3-23 | IGSF6 | TM4SF19-AS1 | TMOD2 |
| HRSP12 | HLA-DMB | RLN2 | PCSK6 | IQUB | TRIP6 |
| RP11-455F5.3 | BLVRB | CD6 | GCAT | NUMB | TNK1 |
| GS1-124K5.11 | C8orf44 | MAP1LC3A | ZNF705E | UPP1 | WDTC1 |
| FAM95B1 | NCF1B | TMEM229B | SAPCD1 | DPH6 | OR7E128P |
| HERC6 | SCML4 | ZNF461 | CXXC5 | ARHGAP10 | PFKM |
| CERS4 | DOCK6 | GPRASP1 | GRAMD4 | BFSP2 | LA16c-329F2.1 |
| RRAS2 | MFSD8 | RP11-485G7.5 | HPDL | NDFIP1 | CNNM3 |
| ARHGEF16 | RP11-563N4.1 | LAMP1 | RP11-395I6.3 | CTSZ | CMTM3 |
| EPCAM | ANKRD30B | MARK4 | SMARCA2 | DLEU1_1 | TUBB4A |
| RP4-738P15.6 | ETHE1 | TMC5 | PRRG2 | MUC4 | MND1 |
| MPST | LINC00884 | RP11-92C4.3 | RP11-38C17.1 | ZNF276 | VPREB3 |
| MAN2B1 | C1orf228 | RP11-96D1.10 | RP11-362K14.6 | TMEM255A | ATAD3B |
| HID1 | MGAT4A | GTF2IP13 | NWD1 | PLD2 | TMEM132A |
| CTC-510F12.4 | CCDC171 | LRRC14B | LSR | OLFML2A | ELMOD3 |
| TP73-AS1 | CLEC17A | MED13L | ZNF579 | ZFP3 | LINC01588 |
| SPPL2A | NFATC3 | TLR9 | SPIRE2 | RP11-77K12.9 | NAV2 |
| TNF | CBY1 | LRP11 | RP11-231C14.4 | RP11-723O4.9 | METTL7A |
| RP11-16E12.1 | LINC01128 | PNPLA2 | BATF | SLC16A4 | RGMB |
| SRC | RP11-561B11.2 | TNFRSF10B | ZDHHC24 | PKD1P6 | FBF1 |
| PROSC | FDFT1 | ENPP3 | USP6 | GTF2IP12 | FAM86C2P |
| TAS1R1 | SLC2A1-AS1 | AP5S1 | UEVLD | NMNAT1 | PLA2G12A |
| BRI3 | DHRS4 | THRA | ZNF876P | ARHGEF39 | CKAP4 |
| CENPBD1 | ZBED2 | LIMA1 | NPPA-AS1 | CDK10 | ACAD8 |
| LACTB2 | RP11-547D13.1 | RP11-325F22.2 | KDSR | NMB | GRINA |
| RP11-660L16.2 | RP11-69E11.4 | TSGA10 | PARP14 | AC024937.6 | SLC25A10 |
| SCLY | IL21R-AS1 | ABHD15 | N4BP2L2-IT2 | DNASE1L1 | RP11-577H5.5 |
| KCNRG | TRIM9 | FAM49A | SLC37A3 | ALDH1B1 | USP30 |
| SPPL2B | SAT2 | FAM86JP | ITGB1BP2 | FBXO6 | TMED6 |
| ACCS | ZNF791 | LEKR1 | ELK3 | SLC16A6P1 | RP11-15A1.3 |
| IFIT3 | RILP | FAM195A | JMJD7-PLA2G4B | FAM78A | AC010524.2 |
| NCF4 | LTA | EPN2 | MAST2 | PSAP | PSPH |
| POLI | BNIP3P41 | HEXDC | LIMD1-AS1 | CTNNBIP1 | GRAMD3 |
| RP11-159D12.5 | LIMD1 | TATDN3 | TTC23 | EIF3J-AS1 | ABCA7 |
| CHIT1 | RNASET2 | ANGPTL6 | RP11-439E19.10 | SNAI3-AS1 | MGST2 |
| EPB41L4A-AS1 | C19orf54 | CD96 | PRF1 | OSTM1 | IL6ST |
| GRM2 | PMS2P5 | NR3C2 | RMDN1 | CYB561A3 | SCART1 |
| SRP14-AS1 | LBHD1 | TECPR2 | NT5DC1 | ATP1A1-AS1 | C21orf33 |
| RNF213 | PECAM1 | SLC2A4RG | ALPK3 | GID4 | SLC4A4 |
| SCHIP1 | RBM26-AS1 | LITAF | IPP | RPS6KA1 | RP11-799B12.1 |
| MYO5B | MCAT | MACROD2 | PARD6A | MYCL | PARD6G |
| UGCG | PRKCD | RP4-717I23.3 | LRRC45 | PLXNC1 | RP11-890B15.3 |
| NR2F6 | IFT46 | POLD4 | OXLD1 | AP000347.4 | RP11-498C9.15 |
| RP11-499P20.2 | AC108488.4 | LINC00174 | LA16c-316G12.2 | RP4-714D9.5 | TC2N |
| FRMD4B | NMT1 | CTD-2636A23.2 | LGALS9 | FBXO4 | SSBP4 |
| ANO9 | ETS2 | ZNF546 | CBWD7 | RP11-545P7.9 | RP11-609D21.3 |
| NLRC4 | FAM83H-AS1 | LINC01585 | RP13-20L14.10 | AGPAT2 | PRPF40B |
| CTD-2135D7.2 | ILDR1 | SUOX | FES | TSEN54 | VPS33B |
| RP11-305E6.4 | SLC22A5 | DDB2 | ZNF682 | TOR4A | MAPK13 |
| ATHL1 | DHCR7 | FAM150B | TMEM106A | CLUAP1 | RP4-635E18.8 |
| GTF2IP20 | INPP5J | CLYBL | RP11-429P3.8 | GSTZ1 | RP11-12J10.4 |
| HIST1H2BN | CD59 | UBXN8 | CDNF | SLC48A1 | LIPG |
| TNS1 | PEX5L | AC009403.2 | A4GALT | RP11-478C19.2 | RP11-360L9.7 |
| SARM1 | SYT13 | IFNG-AS1 | POLR3H | GIPR | IRF5 |
| RP11-747H7.3 | SPTBN2 | GRB7 | ACAD10 | PLEKHF1 | QPRT |
| SLC35G2 | XAF1 | MSX1 | ZNF490 | RP11-218M22.1 | AC073133.2 |
| AACS | RP11-768B22.2 | HYPK | RP11-526I2.5 | OCEL1 | OMG |
| RP11-274B21.1 | FBXO36 | PNPLA7 | LINC00888 | NLRP7 | SMIM22 |
| GINS4 | RAB19 | MSH5-SAPCD1 | RP11-288H12.3 | TESC | AC074212.5 |
| CTD-2036P10.3 | RASA4 | FSTL5 | DICER1-AS1 | CLHC1 | DYNC1I1 |
| MUC16 | GADD45B | SULF2 | MSH5 | CSNK1E | CRYGS |
| LINC00910 | ESRP2 | SIL1 | CNNM4 | MIR9-3HG | STOM |
| AGTRAP | SNTA1 | MIR210HG | MIOX | PPM1N | CFAP70 |
| FAXDC2 | SOX8 | PPP3CB-AS1 | B3GNT7 | NAPSB | COL9A2 |
| EMP3 | CCDC159 | AMT | LAT | SAMD10 | C2orf15 |
| SLC29A4 | ZNF540 | METTL20 | NEK6 | NLRX1 | RRBP1 |
| MET | ACKR4 | ID2 | RP11-388P9.2 | LINC01569 | RP11-96D1.11 |
| RP11-473I1.9 | SELPLG | PARP3 | DYNC2LI1 | RP4-671O14.6 | MAP3K7CL |
| ATAD3C | CD82 | CCDC110 | BAZ2B | AP001625.6 | RAC3 |
| SPHK1 | APLN | KDM4B | NAALADL2-AS2 | CABLES1 | PIGV |
| DLGAP4 | C4orf22 | SESN1 | TNFSF13 | AC007620.3 | USP18 |
| ADAM28 | RP4-813D12.3 | HMOX1 | ZC2HC1C | TRIM16 | USP3-AS1 |
| ALDH6A1 | CTC-510F12.6 | CD151 | PPARA | MKS1 | ZNF577 |
| RP11-326C3.2 | FAM63A | CLCN2 | AHRR | FAM26F | TMCC1-AS1 |
| BATF2 | RREB1 | BMS1P4 | COL9A3 | CCDC78 | AGBL2 |
| BANK1 | LINC01485 | TMED7-TICAM2 | MARVELD2 | LDLRAP1 | SLC46A1 |
| FRG2B | PCK2 | ACY1 | KIF26B | IFIT1 | ZNF780A |
| RP11-449P15.2 | SLC16A10 | ZNF826P | NPIPB6 | ITPKB-IT1 | SULT1A1 |
| FAH | C7orf50 | PLD3 | PARVG | ZNF404 | SLC23A3 |
| KCNAB3 | UBE2O | DMXL2 | TBC1D24 | GLIPR2 | RP11-809C18.3 |
| TMEM63C | PRSS53 | GNB4 | CH5O7-42P11.8 | IFI35 | MATN1-AS1 |
| RNF32 | PLCD3 | DAPK2 | PTAFR | MAPK10 | MYRF |
| DEF8 | MMAB | ITGB1P1 | MYO1C | GOLGA8R | RTBDN |
| FBLN5 | DHRS2 | ZNF239 | HSBP1L1 | GOLGA8A | IGHD |
| AC007386.4 | FAM46C | DNAH6 | SLC17A7 | LY96 | FADS2 |
| TNFRSF1A | SEMA3F-AS1 | LYSMD2 | SH2D5 | DAPL1 | MAML3 |
| HNRNPLL | C6orf223 | CAMKV | RP11-320N21.1 | AGAP10P | B3GNTL1 |
| LAIR1 | PPP4R4 | AC144831.1 | C11orf85 | DDIT4 | PLAU |
| FMNL3 | LRRC25 | AP001057.1 | BTBD6 | SGPP1 | NUDT8 |
| KLHL23 | HPGD | DUSP16 | FAM210B | AP001058.3 | DUSP19 |
| ZNF318 | LHPP | UBE2Q2P1 | TMPRSS15 | HFE | SH3BP2 |
| METRNL | LAMTOR5-AS1 | UBAP1L | IFI30 | LINC01104 | TMEM143 |
| TBC1D4 | CRYBB1 | OLMALINC | AP001046.5 | SAMD7 | ENKD1 |
| GRN | STAC3 | RP11-85B7.2 | THEMIS2 | PINK1-AS | ECE1 |
| GPRIN3 | ITGA10 | PCAT7 | IFITM2 | KIAA1257 | KB-1572G7.2 |
| RP5-965G21.3 | RYR1 | LL22NC03-75H12.2 | TCF7L2 | SCARB1 | BHLHE40 |
| RP11-159D12.2 | RP11-1149M10.2 | ITPRIPL2 | DFNA5 | ZNF486 | SLCO4A1 |
| MYT1 | EIF2AK4 | ZNF793 | SOAT2 | NKX6-3 | XBP1 |
| REPIN1 | ADCY7 | RBM5-AS1 | AC073133.1 | GNG7 | GPR34 |
| PFKFB4 | ITGB1 | RAB3D | RP11-403A21.1 | NCALD | FRG2 |
| CYBB | ZNF570 | EVI2B | LAMA5 | C16orf54 | CTTN |
| MAPK11 | ADM2 | PTK2 | C1orf186 | IFI27 | MMRN1 |
| GALM | AC092580.4 | CD1A | VEGFA | PTPRG | EYA4 |
| ENTPD6 | SLC38A5 | SLC12A4 | GYLTL1B | FAM65A | IFI44 |
| IGHV1-24 | ICAM1 | GNG11 | LINC01021 | PAG1 | TPM2 |
| ADARB2-AS1 | PYGB | TRAF3 | NINJ1 | ANK1 | GLRA3 |
| ISYNA1 | SLC15A3 | ERI1 | RPP25 | LRRC32 | SPSB1 |
| MST1R | ACSS1 | SPOCK3 | CCDC85A | OXR1 | KIAA0930 |
| IFITM1 | POF1B | PRTG | NTN1 | CSRNP3 | MYBPC2 |
| CDKN1A | DENND6B | FBP1 | DIRAS1 | IL21R | ACY3 |
| DENND3 | RP11-482H16.1 | FNBP1 | TTC39C | PLXNB2 | CD9 |

TABLE 4
Proteomic Data: Changes in protein expression in OCI-Ly1-S vs. OCI-Ly1-R

| | | | | | |
|---|---|---|---|---|---|
| P21926 | P09467 | Q96RU3 | Q96HD9 | Q8N584 | Q6K0P9 |
| Q5TEJ8 | P09326 | O15031 | Q9Y6K8 | Q5EG05 | Q96C23 |
| O00182 | E9PF32 | Q8TF42 | O95340-2 | Q06278 | Q96BY6 |
| Q92542 | P42892 | Q05397-5 | P05362 | Q9Y228 | Q9UKB3 |
| J3KNB4 | O43252 | Q6ZVF9 | J3KQV0 | Q6JBY9 | Q16566 |
| P11216 | Q9UPR0 | Q6ZTQ3 | P11217 | Q16880 | P04211 |
| Q5H945 | Q92974 | P06737 | Q15126 | Q16666 | P29466 |
| Q8TEA8 | O15460 | P35610 | Q86WJ1 | Q92766-2 | Q15121 |
| O14862 | Q5R372 | P51858 | O60262 | P01880-2 | Q86VW0 |
| Q9Y2Q0-3 | Q86TP1 | Q14644 | Q9P2K8 | P55265-2 | Q96L92 |
| Q9P2K5 | Q14247-2 | P55265-4 | P15104 | Q7Z3K3 | Q9BZ72 |
| Q7Z6I6 | P26447 | Q9BTT0 | P27540 | O95848 | O95994 |
| P04839 | P61024 | Q86X27 | Q92733 | O60346 | Q6ZS17-4 |
| Q9BT43 | Q9ULH0 | O15020 | G3V3T2 | Q10471 | Q6NXT6 |
| P48960 | Q5VUA4 | Q9Y6Y0 | P11137 | Q14249 | Q9C0C9 |
| Q9UNI6 | Q15306 | P30041 | Q8WV74 | Q15418-2 | Q00534 |
| P16930 | Q5VT52-3 | Q68EM7 | Q68EM7-2 | Q8N1G0 | P07738 |
| P42684 | Q9Y3C8 | P09601 | Q5T5H1 | Q9P0B6 | Q8IZE3 |
| P07355-2 | Q05823 | Q8NFU3 | O75663 | Q8WZ42-6 | O00584 |
| Q99541 | Q86TG7 | Q5VSY0 | Q5SW96 | Q86U28 | P05556 |
| Q5VT52 | Q9H869-9 | O14828 | Q9BUK6 | Q14814-4 | Q53GL0 |
| P14324-2 | Q9BWG4 | Q16555 | Q8WWI1-3 | P48382 | Q8NCW5 |
| P29353-3 | Q96LR5 | Q9P2E9 | Q9P2E9-2 | Q9NPH2 | Q7Z7E8 |
| Q9NX05 | Q8N6N7 | Q8WVV4-1 | P52758 | P06753-3 | P06753-6 |
| Q15149 | Q8N0V3 | P50440 | Q9H4A5 | P05771-2 | P11137-4 |
| Q9UM54 | Q9UHB6-4 | P11047 | Q8N6R0 | G5E9Q6 | Q13568-2 |
| E9PL24 | Q9H869 | Q9NXJ5 | C9JG80 | Q8IW45 | P25445 |
| Q16222 | A0PJW8 | O15231-6 | Q9BXL7 | F8W6H6 | P30043 |
| P35520-2 | Q13772 | P50440-2 | Q8TB37 | Q86TG7-2 | Q8N9I9-2 |
| P15428 | Q96DA2 | Q5R372-5 | Q5VZK9 | Q86V21 | Q9H2G2-2 |
| B5MCA4 | Q9BR61 | O75600-2 | Q07817 | P06753-2 | Q07065 |
| Q16822 | Q00013 | Q05655 | Q9UKU7 | P49189 | P14854 |
| Q8N465 | P26440 | Q9H5K3 | Q15052 | Q9BUL9 | Q96QF0 |
| Q9UHB6 | Q9HBG7 | G3V5T0 | P21854 | Q9BTZ2 | O60504 |
| P07919 | Q9Y6H3 | Q86X76-2 | O94964-2 | Q9Y6K5 | Q8NFQ8 |
| Q96S99 | Q8NFF5 | Q92535 | Q9P215 | Q8N4S9 | P29350-3 |
| Q9Y5K3 | Q9NZ45 | Q15427 | H0YKM0 | Q15654 | F5GXK8 |
| Q93050-3 | Q7L9B9 | P51570 | Q9UIB8 | Q9Y6X9 | Q53EL6 |
| P29353-6 | P25942 | F8W726 | O43291 | Q9UQR0 | Q9UKT5 |
| Q6ZMZ3 | Q96JY6-5 | P31641 | Q8WXI9 | Q9BR76 | O95568 |
| O00764 | P15121 | Q14157-5 | M0QXB5 | Q8WVB3-2 | O00159-3 |
| P35080 | Q9BZD4 | Q9UBF8 | Q14157-1 | Q8N9M5 | P49757 |
| Q9UBQ7 | Q9Y3D6 | P12109 | O95466-2 | Q8N4P3 | P86397 |
| P49585 | Q96G23 | Q99797 | O94985-2 | Q9BWE0-4 | Q7Z2T5 |
| O95236-2 | Q14451-3 | Q14353 | O00560 | F5GX28 | Q96S90 |
| P82921 | P19174-2 | P51965 | Q9BZQ6 | Q8WV28-3 | Q9NVQ4-2 |
| P19174 | Q9BV40 | P54819 | Q92522 | Q7Z7K0 | Q9HCU8 |
| Q9UBR2 | Q7L513-10 | Q9UPR3 | Q92934 | P00813 | P52732 |
| Q9H0F7-2 | Q02818 | P09668 | Q8NDH3 | P23743 | Q96DP5 |
| Q96CV9 | O43405 | Q9BWL3 | Q8WV28 | Q8TAT5 | Q15758 |
| P50238 | P61925 | P11049 | P28288 | Q5VT06 | Q12955 |
| P13674 | Q12955-4 | Q9BYN0 | P20592 | Q7Z4W1 | P0CG34 |
| P31995 | Q9UKA9-4 | Q9BPX5 | Q14766-3 | Q8NI37 | Q9H0P0-1 |
| Q6V0I7 | H0UI06 | Q9BUI4 | Q9Y5B8 | Q9HB71 | O00115 |
| P34910-2 | P14859-2 | Q96ER9 | P10606 | Q9H1E5 | P32942 |
| P09493-10 | O60568 | Q8NBN7 | O43920 | Q4G0N4 | P51452 |
| P13674-2 | Q99519 | P35270 | Q06481 | Q9Y5J9 | Q6UXH0 |
| Q9NYJ1 | Q14764 | Q01415 | Q8NF91 | Q8NFV4 | P20273 |
| P62256 | B4DNW0 | P28799 | Q12873-3 | Q12873-2 | Q765P7 |
| Q5T3I0-3 | Q9NVI1 | P53778 | Q9H3D4 | Q8IV08 | Q58WW2-3 |
| P82912 | P53602 | E7ESJ3 | O94826 | Q9Y606-2 | P30042 |
| Q13946 | Q9H223 | O15264 | Q8IWC1 | Q6P996 | Q9HBH0 |
| O75506 | Q5HYI8 | Q9Y624 | Q96DU3 | P19971-2 | Q9UBL0-2 |
| Q9H497 | Q8ND83 | Q9NV96 | Q56A73 | P16152 | O95817 |
| Q9BQE5 | Q99496 | Q6ICG6-2 | Q8IUH5 | P51553 | P11388-4 |
| Q14153 | Q86WX3 | P57086 | P00167 | P40855 | O75844 |
| A6NJ78 | Q14894 | Q9BXS9 | Q9BX95 | Q15047 | Q6UUV7 |
| P04424 | Q5JTJ3-3 | Q02880-2 | Q9Y2Y0 | P05129 | Q15596 |
| Q969P0 | Q9NQT8 | Q96MW7 | P21291 | F8WCP2 | O14777 |
| O00418 | P04844-2 | Q9BTK6 | Q9H4A4 | B5ME60 | Q96LB3 |
| P34949 | P56381 | Q9BW92 | Q8NDB2 | Q8NBM4 | Q7Z7K6 |
| Q13761-2 | Q8IW35-2 | G3V3G9 | Q9NR45 | Q9Y6R0 | Q8N6F7-2 |
| Q92540 | Q8IWL3 | O14921 | O14936 | Q5T440 | P35219 |
| Q9NX61 | Q9BX40-3 | Q15274 | O43819 | Q9P0P0 | P52630 |
| O75976 | O94762 | O43924 | Q96PU8 | O14773 | P16219 |
| Q9P016 | Q8TAD7 | Q13496 | P56377 | Q5SQP8 | Q13795 |
| Q99653 | P98194-3 | Q9Y657 | Q9NSY1 | Q8IXU6 | Q9NXH8 |
| P40200 | Q96RD7 | Q9UK58 | Q9BWW4 | P07339 | Q99447 |
| P39880-3 | A6BM72 | Q9UPW6 | Q8N2K0 | Q13418 | Q96L93 |
| Q8IUC4 | Q460N5 | P04637 | Q9BUP3-3 | P53365 | Q96DT6 |
| O95716 | Q86UU0 | Q9UP95-7 | Q9NRZ9 | Q96NT1 | Q9UH65 |
| Q99574 | O15164 | Q4U2R6 | Q9UF12 | Q6PIW4 | P18850 |
| Q96K30-3 | P43307 | P82909 | Q9Y5P8 | F8WCZ3 | P35580 |
| Q53ET0 | P11279 | Q6UWP2 | P09669 | P35580-2 | Q9UDY2-3 |
| Q3T8J9 | E7EQA9 | P67936 | P55263-2 | Q9H6T0 | P13284 |
| O96006 | P50053 | O75648 | P17252 | Q9Y5V3-2 | Q9UDY2 |
| Q9HBI1 | P17947-2 | Q9UNF1 | O75116 | Q9UQ13 | Q9HA82 |
| Q7Z5K2-3 | Q8TEM1 | P55263 | Q14765 | Q9H7X7 | P28290 |
| P47985 | Q9NRP2 | Q96MH2 | Q9BRK5 | Q9Y287 | Q8TDY2 |
| Q9NRR5 | Q9NRD1 | P04792 | P19367 | Q6PGN9 | Q9BV29-2 |
| P41212 | P32189 | Q08211 | Q5T9A4 | P51970 | Q9HBZ2-2 |
| Q02535 | P07602-2 | P56211 | Q8NHH9 | Q86VS8 | Q1RMZ1 |
| P49917 | O95168 | O43752 | O14745 | Q9Y2H0 | Q99828-2 |
| O15397 | Q70UQ0-4 | O14950 | P49914 | Q7RTP6 | Q9BRT3 |
| Q9BQC3 | Q9BXW9 | Q6ZRV2 | Q9GZT9 | Q96M86 | J3QRS3 |
| Q6DD88 | Q8TCD1 | P37173-2 | P09211 | P07602-3 | Q9NS18-2 |
| A6NKB5 | Q5BJD5 | Q9H4X1 | Q6P3R8 | Q9BWG6 | A6NHZ5 |
| P62805 | Q02446 | Q5VV89 | Q9P031 | P51797-5 | Q5JTV8-3 |
| Q99081-3 | Q9H9P8 | Q9Y375 | Q08380 | Q07820 | Q6PID6 |
| Q8IVS2 | P49796 | Q8WX94-3 | Q12846 | P43304 | Q6YN16 |
| O75570 | Q49B96 | P15954 | Q9H6S3-3 | Q9UBH6 | Q53FA7 |
| O43399-7 | Q9UEW8 | Q92506 | P35914 | Q8TAE8 | Q5TA31 |
| P54274 | A6NFY7 | P46013-2 | O75879 | Q5TBB1 | Q15080 |
| O75410 | Q9BV86 | Q8IUI4 | Q6PJ69 | Q63HN8 | Q9BZJ0 |
| P38117 | Q8N5I2 | Q13233 | Q13114 | Q14114 | Q8N0X4 |
| Q9UPU7 | G5E948 | Q8N4C8 | B7WPA5 | Q9UBN6 | Q96EL3 |
| Q12983 | B8ZZS0 | O14949 | Q9H6L2 | Q9H173 | J3QTJ6 |
| Q02338 | Q8NHY2 | Q8NE86 | O75386-2 | Q15438 | P46013 |
| Q96BR5 | P42081 | Q01151 | Q96AQ6 | Q9Y2Z9 | Q5TCU8 |
| P0C869-6 | P01857 | Q02363 | P18859-2 | Q92466 | Q96KB5 |
| P49821 | Q9NP66 | B1AMI2 | Q6PIU2-2 | Q9Y244 | O15062 |
| P52888 | Q5JTW2-5 | P83916 | O94768 | P09132 | Q9UBK9 |
| J3KTA1 | P18085 | Q9Y4G2 | O75379 | J3KNA0 | Q8TEQ0 |
| Q96AX9-14 | Q9H3S1 | Q8IYS1 | Q86SZ2 | O95819-3 | Q9H211 |
| Q9Y6M9 | Q9NPF0 | Q9NRW7 | E7EN19 | O15173 | B5MCF1 |
| P04844 | Q9Y3D8 | Q9BUB5 | Q96C10 | Q53S33 | P81877 |
| Q96E52 | Q8N5G0 | Q9NX74 | Q9UJ70 | Q14186 | Q8NDN9 |
| Q9Y5L4 | Q9NQH7 | Q96ED9 | Q6IC98 | Q14766-4 | P48060 |
| Q8WW01 | Q9H7Z7 | P57772 | O75177 | Q9HC35 | O75081 |
| A6NHX0 | Q08357 | Q9UGM6 | Q13948-2 | P23434 | Q9UL54-2 |
| P53004 | O75064 | Q8WVM7 | P46926 | Q92622 | Q00796 |
| Q8N183 | Q14865 | O60783 | Q9H334 | Q14244-7 | Q9UBL0 |
| A8MYU2 | Q9P2A4 | P01861 | A6NDG6 | Q8TCG1 | P37235 |
| P49760 | Q8NG31 | Q92503-2 | Q3V6T2 | P32455 | Q3V6T2-3 |
| Q16877 | Q9NY61 | Q5JTV8 | Q9Y2R0 | Q8IYN2 | Q9BV57 |
| P32119 | Q9BVS4 | Q9UKW4-4 | Q6VEQ5 | Q9Y4E1-4 | Q9BYC8 |
| O75928-2 | P31321 | Q96FJ0-2 | Q99798 | Q9HAJ7 | Q14667 |
| P60981 | Q8WVC6 | Q9H4B6 | Q6UWS5 | Q9GZS1-2 | Q96N67 |
| Q9ULP9 | Q8IVG5 | F5H1U9 | Q14687 | Q96C92 | Q8IUF8 |
| X6R8A1 | Q8NFZ5 | E9PGC8 | Q92820 | Q32MZ4-4 | Q8IUD6 |
| O43665-3 | Q9UPT9 | Q9UI08-2 | P08574 | Q9Y5X2 | O00220 |
| Q15050 | P56180 | Q9UGU0 | Q13835 | Q96I15 | P42336 |
| Q99757 | Q9NRZ7-3 | P00403 | Q5JVS0 | Q9BUT1 | P20674 |
| P62324 | P12883 | Q9Y2Q9 | Q9H967 | O75817 | Q9HD23-4 |
| P36957 | Q8N0S6-2 | Q9UBV2 | Q86YH6 | Q9NRF8 | Q7Z7K6-3 |
| E7EPN9 | P56945-6 | A8MXV4 | Q70EL1 | Q6GQQ9 | Q9UFW8 |
| Q9BTE3 | Q9Y520-7 | O14513 | P08240 | Q9Y2H1 | P09972 |
| Q9P035 | Q9BV79 | Q8NAF0 | P82970 | P53634 | B4DJM0 |
| Q13541 | Q12765-2 | P49335 | P21912 | Q9NQA3 | Q9NWR8 |
| P58004 | O43488 | Q7Z7A4 | Q9BTT6 | P35568 | Q641Q2 |
| Q8TB61 | Q5JQC9 | B1AM49 | Q6UW78 | Q9ULL1 | P22830-2 |
| P10244 | Q8N884 | P53675 | Q9NRD5 | Q9UQ80 | Q8WWW0-2 |
| Q96IJ6 | Q9Y3E7 | A6PW57 | Q8WVN8 | Q9Y5J6 | Q8N8N7 |
| P22570-7 | Q9Y2H2 | Q63ZY3 | Q9NQ88 | Q6AI08 | O95182 |
| Q9UHA2 | P61011 | Q9HC07 | P30040 | Q14571 | P29401-2 |
| Q7L8W6-2 | Q86T24 | E7EX70 | P82914 | Q9Y3C1 | Q96BK5 |
| Q9BSI4 | Q8WY36 | Q96CG3 | Q9UII2 | P42680 | O00161 |
| Q8N8Y5 | Q16658 | Q66K64 | P23381 | Q9BYC5 | O94900 |
| O95983 | P31040 | P51668 | P28074 | Q9H3H1 | Q8N2F6 |
| Q8IWJ2 | P10114 | Q96RL7 | O15446 | Q96EY8 | D6W5Y5 |
| P19532 | Q9UNE7 | Q9P2M7 | Q9P0S2 | P06493 | Q01085-2 |
| H3BP20 | P04062 | Q8N4T4 | Q9NWS6 | P37802 | P0C7P0 |
| O60733 | Q68E01-2 | Q9Y6I4 | P25325-2 | P09936 | Q9BYN8 |
| Q8N726 | P01344-3 | Q14693-3 | Q14005 | Q9HCM1 | Q9NTG7 |
| A6NIH7 | Q3ZCW2 | O95243 | O94855-2 | Q15554-4 | P49593 |
| O95299 | O00330 | O14548 | P50995 | P15056 | Q7LFL8 |
| Q9BYX2 | Q5VZZ6 | E7ETE0 | Q8N983-4 | P56181-2 | H0YH15 |
| Q86WQ0-2 | Q9BX40-2 | Q8TAK5 | Q9P2H0 | P41440 | P18206 |
| Q96P48 | O95251 | Q13158 | P31930 | P57735 | Q96GC6 |
| Q13342 | Q9Y619 | P52306-4 | Q9H4I2 | E9PRZ1 | P50213 |
| F5H1Y4 | Q13939 | A7XYQ1 | Q96PE7 | Q96IQ9-2 | Q9NX58 |
| Q9UIL1 | Q9Y3Z3 | Q16143 | P35813-3 | G5E9A6 | P46977 |
| Q01085 | P42166 | Q9BSH4 | P52306 | Q9BUZ4 | Q9NWQ4 |
| Q86XX4-2 | O95478 | Q96B70 | Q9Y291 | Q9HBM1 | O75380 |
| K7EPM4 | Q9HD33 | Q96CM3 | Q9Y315 | Q9NP58 | P52815 |
| Q9UQE7 | Q9NVV0 | Q8TCT9-2 | Q9NYP9 | O95235 | Q96T49 |
| Q00987-11 | O00762 | Q9UJZ1 | P03928 | P68431 | P42696 |
| Q13325 | M0QXA7 | Q16635 | P32929 | Q9H0U6 | Q16775 |
| Q9NZJ6 | Q9H3S4 | B4DTU4 | Q06546 | Q86U90 | Q8NEN9 |
| Q9NRW4-2 | O94880-2 | Q9BWC9 | F5H211 | Q9P1F3 | Q96CN5 |
| Q6PII5-2 | Q9GZN2 | P53396 | P42081-4 | Q6P1X6 | Q32MZ4-2 |
| Q6P161 | O43709 | P50552 | Q8NHG8 | Q96JZ2 | O00471 |
| Q8TDZ2-4 | Q96C19 | Q9NTI5 | Q8TE49-2 | Q5T1R4 | P18858 |
| Q9ULV4-3 | Q6P0N0 | F8WEC0 | Q9H0U3 | O95352 | Q96EU6 |
| Q06830 | Q70UQ0 | O75683 | Q17R31-3 | P31937 | Q7Z628 |
| Q13402 | Q969T7 | O43278 | Q96A19 | Q14011 | Q6DHV7 |
| A3KMH1 | P17568 | Q92667 | Q5RI15-2 | Q8IZK6 | Q96N06-2 |
| Q6P087 | Q6P0Q8 | Q9Y483 | O94889 | P53396-2 | Q6JQN1 |
| B1AKJ5 | P55789 | Q9H9L3 | Q5XUX1 | Q5TA50 | Q9Y3L5 |
| Q9NZM3 | Q9BZE1 | P26842 | Q15283 | Q8TCA0 | Q8NAX2 |
| P78330 | P33552 | P13861 | Q9Y3B9 | P52564 | O75145 |
| Q93052 | Q9H4L7-2 | O96000 | Q9P0M9 | O15305 | Q5T6F2 |
| O94776 | P51159 | P07237 | P32971 | Q8TAP8 | Q9H6Q4 |
| O94880 | O15049 | Q96MG7 | Q53H80 | Q9NX62 | Q96NL6 |
| Q8ND82 | Q8IUW5 | Q9BYC9 | P40926 | Q8WVK7 | O76075 |
| P36871 | Q53H54 | P29590 | Q96I51 | Q9Y3A2 | Q6ZS86 |
| Q9NX14-2 | Q9BW19 | Q02156 | Q8N4T8 | P17275 | Q9NSI2 |
| Q9NS87 | Q9UKA9 | P29590-2 | Q96NL8 | Q96KA5 | P51151 |
| Q8IVH2 | P50579 | Q9UI26-2 | Q09019 | Q8NHP6 | O95202 |
| P31150 | Q96A49 | Q7L2H7 | Q13084 | Q9NXA8 | Q8TB36 |
| Q96JM3 | Q9UHA4 | Q9HAT8 | B0QYS7 | P45973 | F8VZ29 |
| P52306-2 | Q9HBH1 | Q9NPB6 | Q9Y6R4 | Q96A57-2 | Q8N3J5 |
| Q969H8 | P11182 | Q9UKA2 | Q9GZY8 | Q9BYV9 | P20020 |
| Q9C0B1 | P30101 | P06576 | Q9Y2E5 | Q9UJU2-6 | P82930 |
| Q9NRX2 | Q8IVF4 | Q16831 | Q7L8W6 | P35237 | P07311 |
| Q99807 | Q99986 | B8ZZC8 | Q99607 | Q9H8M7 | Q12857-4 |
| Q9BZG8 | Q9UKK3 | Q9NS69 | A4D161 | Q3ZCQ8-2 | Q3KQV9 |
| O75530 | O95298 | Q12907 | F8VQZ7 | Q9ULU4-16 | Q15181 |
| Q9H160 | P09622 | J3QRU1 | P50336 | O75845 | Q9UBF2 |
| P16083 | Q9NP72 | Q9UIL8 | Q9HA77 | Q8N3U4-2 | Q9ULE0 |
| Q9Y639 | Q9BSD7 | Q6PK18-2 | Q99996 | Q07617 | Q8N5I9 |
| O43304 | P31350 | Q9BSH5 | Q9NRP4 | A9Z1Z3 | Q9Y613 |
| P08758 | P82673 | Q96HQ2 | P15924 | P56182 | Q9BX68 |
| Q5W0V3 | Q9Y2Q3 | Q9NQW7-3 | Q9NXF1 | Q8NDD1-3 | P61604 |
| Q9Y692 | Q5VUM1 | O00410 | O95197 | O95789 | J3KNW7 |
| Q9BYT8 | P53985 | Q92665 | P01130 | Q9Y680-2 | P23921 |
| O43837 | P02461 | Q9UJU2 | Q70Z53 | E9PJD7 | Q9H6P5 |
| O14792 | Q14643 | Q32P28 | Q6NY19 | Q6P1K2 | Q15772 |
| Q9HD45 | Q9ULU4-19 | Q13571 | Q8N2Z9-3 | Q9Y617 | Q9NPJ3 |
| Q15345 | Q9BVL4 | Q13136 | Q969S9 | Q03393 | E9PB38 |
| Q5K651 | Q8TES7-6 | Q9NRW3 | O60344 | Q9HAV7 | Q86UW6 |
| Q8WWQ0 | Q6PML9 | Q8N567 | Q9H9A6 | P30044 | Q99717 |
| Q9BZK7 | Q86YP4-3 | Q9NQ50 | P27105 | P32321-2 | Q14681 |
| O95831 | O00255 | O43187 | H3BMM1 | P56937 | Q6IN85 |
| P51532 | Q5JUR7 | P13498 | Q92520 | Q6PD74 | Q9BQ75 |
| Q96AT9 | P22415 | Q02750 | Q02809-2 | Q96MT7-2 | H0YL11 |
| Q96HE7 | P31323 | P98174 | O95861 | A2A288 | P55011 |
| Q8IWF2 | P22695 | Q17RY0 | Q9NXG6-3 | Q9NRN9 | P62851 |
| Q6ZU35 | Q6UWZ7 | Q969M7 | P52657 | P42765 | Q14204 |
| Q9Y4K1 | P19338 | Q14019 | Q8NFD5-3 | P17707 | Q9UKI3 |
| Q9Y2D5 | B2RTY4-4 | P01137 | Q96GY0 | P49023-2 | Q15004 |
| Q969V3 | Q9UHQ1-2 | Q14678 | Q8N3R9 | Q8NE65 | Q9BVV7 |
| Q9UGP4 | E7EMS2 | Q9NZJ9 | O14967 | Q9BVJ6 | Q9NZI7 |
| P16278 | Q8TD55 | Q9UK23 | Q6P4F2 | Q09327 | Q9UNA4 |
| Q9H8H2 | Q86YS7-3 | Q96LC9 | Q6P1Q0-7 | Q9BV20 | Q96B01-3 |
| G8JLL9 | Q92769 | Q6BCY4 | Q13480-2 | Q9NR48 | Q9H0Q0 |
| Q4G176 | Q6ZS81 | P82933 | Q86TM6 | Q13303 | Q13303-2 |
| Q9BQ70 | Q8WUB8 | Q9NYB0 | P46109 | P14598 | Q99442 |
| Q15172 | Q14684 | Q6IN84 | Q9Y385 | S4R3B8 | O43719 |
| Q8TDP1 | P42898-2 | O96028 | Q8NF37 | Q96A72 | P57081 |
| P48507 | Q6PL18 | B9A057 | Q7Z7F7-2 | Q9BY77 | O75475-3 |
| Q92538 | O60831 | Q9UGI8 | O15040 | Q8TEH3-6 | Q9NXV6 |
| Q9Y5J7 | Q15555-5 | K4DIA0 | Q8NEF9 | Q9NP73-2 | Q6PGQ7 |
| Q8IXT5 | Q7Z3C6 | Q7RTS9 | Q96G01 | O43464 | Q9ULC5-3 |
| Q04837 | P14316 | O60264 | P56277 | Q6ZNB6 | P08133 |
| P46063 | Q9NWK9 | J3KN01 | P48047 | G5EA39 | P49795 |
| Q15554 | O95749 | P61009 | Q9BUN8 | Q15645 | P51617 |
| Q14061 | O75915 | P82675 | Q9NRX4 | Q9UMX5 | Q149N8 |
| P35232 | Q9NXH9 | Q9NRW4 | Q96C01 | P06746 | Q9H6E5 |
| Q9UHK0 | I3L0E3 | U3KQL2 | Q96EB6 | P17050 | Q5MIZ7-2 |
| Q8TAQ2 | Q9H668 | Q14839-2 | P51692 | E7EQ64 | P14921 |
| P24752 | P35579 | A0JNW5 | Q9Y3D3 | Q96GX5 | Q9ULE6 |
| P24941 | P35558 | P19404 | H3BSW6 | F5H006 | Q96D71 |
| P10599 | Q9Y6B6 | O43716 | Q9H4Z2 | O15235 | Q8NC42 |
| Q9HCH0 | Q9UBB4 | F8VX04 | Q4VC31 | P80217-2 | Q9BYX4 |
| Q330K2 | Q9NVI7-2 | Q5ST30-4 | Q9H019 | Q8TAF3 | Q02218 |
| P35221-2 | O15160 | O14576 | Q6ZRI6 | E9PD98 | Q0VF96 |
| Q8TEA7 | O14757 | Q9UHD9 | P18621-3 | Q96BD8 | O60220 |
| A6NI72 | P43378 | O75971 | Q86YH6-2 | P00492 | O75143-5 |
| Q6UXN9 | F8VXC8 | Q70IA6-3 | Q8NEC7 | Q68CP9 | Q12912-2 |
| Q9NV79 | Q9Y4A5 | P08107 | Q969S3 | P49069 | Q14197 |
| Q86U86 | Q9Y4C8 | Q5T4U5 | P00451 | O95696-2 | Q9BPX6-3 |
| Q00526 | A2VDJ0-5 | P25774 | Q8WWV3 | Q8WYN0 | P41247 |
| Q8NI35 | O95336 | Q8NDD1 | O76080 | P04818 | Q9Y2Y9 |
| Q96GK7 | O60664-3 | Q86WR0 | Q9Y4B4 | Q14149 | Q96IR7 |
| Q969H6 | Q9BWT6 | Q14683 | P27824-2 | P13598 | Q96KC8 |
| Q53EZ4 | Q9Y242 | Q12912 | Q13287 | Q8N766 | P15374 |
| P53621 | Q9GZT4 | Q9Y5N6 | Q9Y232 | P16402 | B7Z1P2 |
| Q8WW22-2 | P13693 | Q96GN5 | Q15649 | O60664 | Q96C24 |
| Q9UJ98 | Q01201 | Q9BRT9 | Q8NC60 | Q9Y5Y6 | Q02252 |
| Q9H5Z1 | Q6PK04 | O95104 | O14737 | Q9BZX2 | Q9UN86-2 |
| Q7L2K0 | O60307 | Q9Y5B6 | Q9NX20 | Q9P2M4 | Q99933 |
| Q96EY1 | Q93084-2 | Q6NTE8 | Q6DN12 | I3L4Q0 | P10586 |
| P15391-2 | Q9BXP2 | Q00341 | Q8N543 | P50748 | Q06210 |
| Q6XZF7 | O43639 | Q13505 | Q9Y3C0 | P16615 | Q8WWN9-2 |
| Q9H7E9-2 | Q9HCS7 | P41162 | O14647-2 | Q9BV90 | Q9Y376 |
| Q92664 | O95379 | P36956-4 | F8VWW7 | Q5XKP0 | P42773 |
| Q13526 | O75503 | Q13085 | P32322 | Q13432 | P20591 |
| O15213 | P23284 | Q9BW60 | Q9BX84 | P38919 | Q96HL8 |
| Q9P1Z0 | Q9NZJ9-2 | O76095 | Q96LT7 | Q3B726 | Q96Q40 |
| P21333 | Q93100-3 | Q9UJK0 | O94903 | Q9UID6 | Q9NXE4-4 |
| Q92618 | O43615 | Q7L523 | Q8IWU2 | Q96EK6 | Q9Y530 |
| P08579 | P10809 | P06748 | Q5JPH6 | O15155 | J3KP28 |
| A1XBS5 | Q6ZYL4 | Q9BYD1 | Q8WWH5 | Q96PZ2 | P36639 |
| Q9HCN8 | Q15714 | Q15771 | Q7Z6K5-2 | O00267 | Q96LL9 |
| Q8WVJ2 | Q9UFF9 | O14519 | Q66LE6 | O43395 | Q8WUX2 |
| Q5JWR5 | Q13185 | J9JID7 | C9JTB0 | P24394 | Q4ZHG4 |
| P04114 | Q8NBR6 | Q9Y6A4 | Q13614 | E9PJ55 | Q15084-2 |
| Q6PII5-4 | P78549 | P26196 | Q96IQ9 | Q86TS9 | O75792 |
| Q9NS23-5 | O43463 | O95900 | Q15773 | O15535-2 | Q14696 |
| Q9UI14 | O95619 | Q92925 | P10415 | P53597 | Q5U5Q3 |
| Q5TEC6 | Q9NYP7-2 | O43818 | Q8IVV7 | Q16763 | Q8N655 |
| P56279 | P15529-2 | P13716-2 | P16333 | Q8WVE0 | Q16891-4 |
| Q8IXM3 | Q9NWU1 | B7ZAQ6 | P14927 | Q9NUI1 | A0A075B730 |
| Q9Y6M7-13 | Q9H0R6 | Q8NEM2 | Q9Y3D9 | P51451 | Q8WWK9 |
| P08651 | Q9H8V3 | Q9Y2Z2-6 | Q8IW19 | Q9Y6E0-2 | Q9UKT9 |
| Q2TB10 | Q9NY97 | O43715 | Q8WWK9-5 | Q9H410 | P62072 |
| Q9UIG0 | Q9NVA1 | O43325 | P48651 | Q9BVG4 | Q6UUV9-2 |
| Q13586 | Q8N9L9 | Q15942 | Q8IUR0 | Q9BQ95 | Q8IUE6 |
| Q96KP4 | Q8N103 | Q9Y5M8 | Q9H1J1 | Q9NZ08 | Q9H082 |
| P41226 | Q68CJ6 | Q15788 | O00566 | Q13151 | P36507 |
| P13804 | Q562E7 | K7ES69 | P56556 | O43566-5 | P39656 |
| Q96C57 | Q9BTV6 | Q8IUH4 | G3V1A6 | P15498 | Q5VV41 |
| Q6P1R4 | O75439 | Q9H8U3 | F5H2X7 | P00387-3 | Q9NWA0 |
| O43566 | O43896 | Q9NS91 | F8VWL3 | P23634 | O75368 |
| Q9H3K6 | P60520 | O75582 | Q96HS1 | Q16891-2 | Q86VQ1 |
| Q8IXK0-5 | O43491 | Q10713 | Q8IYM9 | Q5TAP6 | P62140 |
| Q00610 | Q6P4F7 | P49902 | Q96CW1-2 | P60866 | P62330 |
| P53007 | Q86X29 | Q13620-3 | Q9NZE8 | Q9BV68 | Q15435 |
| Q6P1N0 | O15294 | Q9Y3B4 | O75884 | Q13442 | P23526 |
| Q5T653 | Q9UI43 | Q8N0T1 | Q5FBB7 | Q2TB90 | Q9Y5S9-2 |
| Q9Y4W6 | Q96DB5 | O75695 | Q92793 | Q9Y5S9 | P61204 |
| B0QY89 | Q9BXV9 | Q8N5U6-2 | Q8NBS9 | Q96SU4-2 | Q9UN86 |
| Q6TFL4 | Q9BY12 | P06733 | M0R226 | Q16531 | Q9UG01 |
| P82932 | Q14674 | O95711 | P82650 | Q9BZF1 | O76021 |
| O60493 | P14635 | P20962 | Q9Y2K7 | Q96JM7 | O94804 |
| Q8TCC3-2 | Q9BX84-6 | Q12986 | Q86XZ4 | P11926 | C9JRZ6 |
| Q15436 | Q9NVS9 | Q6PJF5 | Q76FK4-4 | Q15532 | Q9Y2K6 |
| O14907 | O00273-2 | Q14257-2 | Q9BXS4 | Q96HM7 | Q5T0D9 |
| P61927 | Q16774 | Q05BQ5 | P30533 | A0FGR8-2 | X6R2S6 |
| O15527 | Q16718 | P30086 | Q13686 | Q6P9B9 | P17096 |
| P26885 | Q01433 | K7EMV4 | Q9GZP4 | Q9H7B2 | P30084 |
| O95997 | Q86UL3 | O60671 | Q562F6 | Q05209 | O00311 |
| Q13126 | Q9H777 | Q8N8Z6 | P25705 | Q92608 | Q8TEK3 |
| Q9NQR7 | Q7Z333-4 | O15270 | P32519 | Q96GC5 | Q14008 |
| P29375 | Q9BSC4 | Q86XI2 | Q16864-2 | O75533 | P62633-2 |
| A0FGR8-6 | Q9NZM4 | P58546 | P04075 | Q9H3S7 | P46199 |
| Q15907 | R4GN35 | Q8N5D0 | H7C2Q8 | Q9Y6A5 | P38432 |
| Q92636 | Q8TBC5-3 | G3V4A6 | O60216 | O43521-12 | Q96CU9 |
| Q29RF7 | P00390-2 | Q04323 | Q69YH5 | O95294-4 | Q96AB6 |
| Q9NZJ7 | Q9BRX8 | Q9NQ55-3 | Q13362-4 | P32249 | Q01082-3 |
| Q9BUE6 | Q8NB37 | Q96MG8 | Q92621 | P78356 | P84101 |
| Q96K76-4 | P26232-5 | Q9P0U3 | Q9H267 | Q7Z7G8 | O00217 |
| Q9UK99 | O95721 | Q9NZI8 | O95167 | Q9Y3P8 | Q9Y2L9-2 |
| Q9UQ90 | P60763 | P17096-2 | Q96LA8 | Q6NX45 | Q9H0M0 |
| P48735 | P52565 | Q15025 | O60907 | Q9NZC3 | G3V5X4 |
| Q86YN6 | Q8ND30 | Q6ZSJ8 | A6NJ78-4 | Q6ICL3-4 | P62753 |
| Q96RT7 | Q969Q0 | Q7Z5L2-2 | E7ETU7 | P43897 | P49411 |
| O75191 | Q71DI3 | Q9UJ83 | Q9Y4B6 | Q9BSF8-2 | P02795 |
| Q9UKG9-3 | Q9Y263 | Q643R3 | Q7Z6M1 | Q9H1Z4 | Q9Y4G6 |
| Q96HA7 | Q5VW38 | Q8NBF2 | P57076 | P28702-3 | Q96DC7-2 |
| Q9BT30 | P07203 | Q9NWB7 | Q5VYK3 | Q8WXH0-2 | Q15208 |
| P56962 | Q9P270 | Q8WUN7 | Q8IZT6 | Q15334 | Q9Y2S6 |
| Q9UNK0 | P54687 | Q13191 | Q9HCE1 | Q9H6K1 | Q6AI12 |
| Q9BSY4 | P61020 | Q9UPW5 | Q2KHM9 | Q5SW79 | Q9Y2G2-5 |
| Q8N3X6 | Q9Y3A3 | Q9Y265 | Q01082 | Q9H6U8-3 | Q9GZQ8 |
| Q9UG56 | Q9H3K2 | D6RIF6 | A8MUP8 | P37837 | Q96EY1-2 |
| P84077 | Q5VZL5 | P82664 | Q9BQ39 | O96033 | Q96E11 |
| Q9Y676 | Q7Z5Y7 | P57105 | P30793 | Q86VR2 | B4DSR2 |
| Q6PFW1 | Q15819 | Q6NVV7 | Q8N531 | Q16787 | Q13263 |
| J3KPV8 | P62253 | P98171-2 | Q96DE5 | P53350 | J3KN10 |
| Q53H82 | Q96QP1 | P24863 | A1YPR0 | P13929 | Q92547 |
| Q9NUD5 | Q9Y584 | Q13616 | Q9Y5K5-3 | O95365 | Q8WZ64 |
| P30405 | Q14657 | Q8NBA8 | E7EV10 | P18075 | Q8TDX7 |
| Q53H96 | A4D1E9 | P15260 | Q5JS54-2 | Q9NR33 | Q99611 |
| Q9HB19 | Q96JJ3 | Q9H840 | O43592 | O76094 | Q13011 |
| J3KPP0 | O14530 | P60174 | O14776 | Q09472 | P35659 |
| Q6UWU4-2 | P62633-8 | Q9NQS1 | Q9UI09 | Q9H078-2 | Q9NWZ3 |
| P04150 | O75438 | Q9Y6E2 | Q15382 | P62829 | P50453 |
| Q9UNQ2 | Q6P2Q9 | Q14142 | O95461 | Q9NR96-4 | Q8N4C6-7 |
| Q96DM3 | Q96SB8 | O15541 | Q15388 | P10242-4 | Q9C0I1 |
| Q13503 | P23786 | P61026 | Q13574 | Q0JRZ9 | O75815 |
| B4DLN1 | Q15022 | Q9BWH2 | Q96K37 | Q15797 | Q8WVB6 |
| Q9UPP5 | Q0PNE2 | Q15185 | Q8TB05 | Q99417 | Q32Q12 |
| Q9H0V1 | Q8TCS8 | Q53T59 | C9JYP6 | Q14165 | Q7Z422 |
| Q8WY91 | Q92674 | P78527 | O14607-4 | O43379-4 | Q2TAC2 |
| Q9HB65 | O43598 | Q8IUZ5 | J3KNL2 | Q9NWX6 | Q9NVZ3 |
| P62380 | P20810-6 | O15066 | J3QL71 | P80297 | Q9NPE2 |
| Q9Y6G9 | O95295 | Q969Y2-2 | O75190 | Q9NYL4 | Q9H5X1 |
| P09960 | Q9H7N4 | P63272 | J3KP27 | Q9H2W6 | P0CG13 |
| Q5SW79-3 | Q96RS6 | Q9NVH6-8 | Q9UNL2-2 | O00154-4 | Q9NZM5 |
| O14657 | Q9NWY4 | P15559 | Q9C073 | Q15906-2 | Q9BXB4 |
| P63173 | Q13422 | A6NIW2 | P15923-2 | O60306 | P42695 |
| Q9Y5K5 | Q3SXM5 | Q6N063 | P09104 | Q8WXX7 | Q9P2Q2 |
| P34931 | O75718 | Q96I99 | Q96DT7 | Q969X5 | Q13487 |
| P42167-2 | Q9NXX6 | Q9NRP0-2 | Q02127 | Q9HCE3 | P49662 |
| O43447 | P29992 | Q96SN7 | Q6Y1H2 | Q5GLZ8 | P61962 |
| Q6PI48 | P51610 | P48729 | Q6IAN0 | Q7L8J4 | Q9H900 |
| Q12968 | P41236 | O60518 | P16949 | O75478 | Q14692 |
| Q9UPS8-2 | Q13422-2 | Q96T76 | P04053 | Q9H5I1 | Q9Y620 |
| Q7Z5L9-2 | M0R2A0 | Q14807 | Q5T6S3 | Q9UL26 | Q9HAV4 |
| Q9NP73 | Q7Z6M3 | Q96JN0 | Q9Y248 | Q6DN14 | Q8TCE6 |
| O75475 | O00186 | O75381 | Q8N0Z3 | Q9UBC2-2 | P98179 |
| Q96LW4 | Q9HBL8 | P13196 | Q8N5Y2 | O75414 | K4DI93 |
| Q13620-1 | Q9Y5A9 | P15289 | Q7L1Q6-3 | P53999 | Q7Z5L9 |
| Q9H089 | O43167 | P53701 | Q9NRA2 | P36551 | Q6ZN55 |
| Q9H008 | Q96JP5 | P49589 | D6RAM3 | P35222 | Q4J6C6 |
| P18074 | Q8IWV7 | O60825 | Q06730-3 | Q96II8 | Q9BQA9 |
| B0I1T2 | Q16774-2 | Q9HD15 | Q9BWU0 | Q6P2I3 | Q05513 |
| Q8N9N8 | O00273 | Q8IYW5 | Q9NZ52 | O43765 | Q7Z7A3 |
| O95297 | Q9UJV8 | P81274 | Q9UH73 | Q2M389 | O60930 |
| Q9NX18 | Q969H0 | Q9H6K4 | Q9UJX4 | P13073 | Q13362 |
| O43809 | P51157-2 | Q9NZR2 | Q9H7H0-3 | Q9NWL6 | Q96AB3 |
| Q9NY33 | P07737 | P11177 | Q9NRX1 | O14933 | Q9BPX3 |
| Q9P0U4-2 | Q9UJY4 | Q14554 | Q9UPN6 | Q9BWT3 | Q86TU7 |
| J3KN32 | Q9NQ89 | Q6ZXV5 | Q9GZT3 | H7BZ55 | Q9UHV9 |
| Q96JH7 | Q9BYJ9 | Q5VW36 | O95822 | Q86XP1 | O75909 |
| Q15782 | P63010-2 | Q14651 | O60315 | Q969X0 | P61088 |
| P50914 | Q9UN19 | Q13464 | Q9H093 | Q12802 | O00165-2 |
| Q8NFI3 | P30622 | Q14527 | Q9H867 | P63172 | O94829 |
| Q9HC52 | Q9UHN6 | O60290 | P41743 | Q9UP52 | Q9BTE7 |
| B7Z9C2 | Q9BUH6 | Q5T5X7 | Q15154 | Q4LDG9 | Q9Y3B7 |
| F5H049 | Q8N3J3 | Q5VWZ2 | P41227 | Q96RG2-2 | Q02930 |
| P07948-2 | O94782 | O00425 | O95347 | P62750 | Q9Y605 |
| Q16134 | Q13405 | Q8WYB5 | Q5JSZ5 | Q9Y5P6 | A6NFQ2 |
| O60488 | Q6P1J9 | Q02447 | Q9BRT8 | P32780 | P09110 |
| O75131 | Q9HBL7 | Q15911 | Q9BU89 | Q8IYA6 | O14986-2 |
| Q96SZ6 | Q9BSL1 | Q9P0R6 | H3BSS5 | Q9Y2X9 | P62891 |
| Q9UMY1 | P23025 | O94967-4 | Q9HB40 | Q8NCD3 | D6RER5 |
| Q9HCC0 | P08397 | P13612 | Q8N108-12 | Q13608 | E7EQ34 |
| Q96KC2 | Q6MZP7 | P48449 | B4DR61 | P49327 | Q6VMQ6 |
| Q01581 | Q5PRF9 | Q14202 | P33981 | Q9BXK5 | Q7Z6K3 |
| Q92830 | P18887 | Q9H074-3 | Q9NXC2 | P05204 | Q5TC82 |
| Q9NYA4 | O43318 | Q9Y6R7 | O15269 | Q96S94 | Q9P2N7-5 |
| Q9H425 | Q8N165 | A6NEM2 | Q9NTJ3 | Q14202-2 | Q96B01 |
| P55209 | O94973 | P00558 | Q6PL24 | Q9NYJ8 | Q14147 |
| P12259 | Q6N043 | Q15041 | Q8WUW1-2 | P35869 | Q8NCN4 |
| Q9Y4P3 | Q8IV53 | P22794-2 | P60510 | Q12931 | Q9H0I3 |
| P24043 | Q969G6 | Q8NBX0 | O00592 | P31942 | O43676 |
| P05386 | P08567 | Q08050-3 | P39019 | Q14CB8-2 | Q92556 |
| O14874 | Q9NRY2 | Q99551 | Q01850 | P28907 | P50851-2 |
| Q9UPY8 | Q9Y230 | Q9UBP6 | Q9UL18 | Q9H0C5 | Q9NTX5 |
| Q01196-8 | Q96B49 | Q6F5E8-2 | Q86VP6 | Q9Y295 | O43674 |
| A5D8V6 | J3QSW6 | Q92785 | O43283 | O60613 | Q9H7F0 |
| Q9Y6Y8 | P50542-2 | O94822-3 | P00973-3 | P16220 | Q96BY9 |
| Q00688 | C9JLH9 | Q92616 | P31327-3 | Q9UHP3-3 | P51817 |
| Q8IU81 | P28838 | P49589-3 | P46736 | Q9H3U1-2 | Q96M29 |
| P41214 | Q99595 | Q9NX70 | P83881 | Q9H9Y4 | Q9Y697 |
| Q5VST6-2 | Q8N3F8 | O15439 | P79522 | O43684 | Q96EH3 |
| P63027 | Q9H2P0 | Q8TAG9 | Q9H6F5 | Q86VD7 | Q9UNM6 |
| Q9UKQ2 | Q5VIR6-4 | O60499 | Q14232 | Q9NUL7 | Q8IUC6 |
| Q9NP61 | Q16206 | Q9Y4X0 | Q9NPH0 | Q96KQ7-2 | P62310 |
| P13798 | G3XAA4 | Q15012 | Q08945 | P56589 | P08237 |
| Q92947 | Q09161 | Q13485 | O75356 | P33121 | Q9HBU6 |
| P42167 | Q9UHJ3 | Q96Q89-2 | Q12888-2 | O75808 | Q9BQ24-2 |
| P16591 | Q6P4I2 | Q6STE5 | Q8NEJ9 | Q8TB03 | Q12905 |
| Q99426 | Q9BXY0 | P09488 | Q99829 | Q9BSY9 | Q5VTE6 |
| P48551 | Q96H22 | Q13794 | Q2TAZ0 | Q6ZRS2 | Q8NDV3 |
| Q16539 | P55010 | P61019 | A2A3N6 | Q9NTM9 | Q9Y259 |
| Q8WUD1 | Q96P53 | Q13469 | Q9BXB5 | Q96G46 | P07108 |
| Q96AJ9 | Q9UBW7 | Q9Y587 | P28331-2 | Q13107 | B7ZC38 |
| Q17RN3 | Q9H4W6-2 | Q14444 | Q96IU4 | P51397 | Q9Y6X8 |
| O96017 | Q9BY27 | O43822-4 | Q9NRR4 | Q96S44 | P60953-1 |
| O95544 | Q8IZC4 | Q96I59 | Q9C0K0 | O95602 | Q9UHD8 |
| E9PHG5 | Q96EY7 | P20023-3 | Q00653 | P62993 | O43318-2 |
| P13995 | Q9Y2U8 | Q9Y296 | Q53RE8 | Q6F5E8 | Q9UGY1 |
| Q96FJ2 | Q8IXJ6 | O75027-3 | O43504 | Q8N335 | Q8IY81 |
| Q96FX7 | Q96BW9-3 | Q8N9N5 | Q8N5G2 | P62306 | A8MTJ3 |
| Q9BSK4 | Q13242 | Q9H8M2 | R4GMX8 | O00506 | O43813 |
| P05198 | Q14592 | Q99683 | P43490 | Q9NW64 | Q8NAV1 |
| Q32NB8 | P61018 | Q9UKA4 | Q16543 | Q9H773 | Q7Z739 |
| Q3KNV8-2 | O94886 | P07948 | O43678 | J3QLS3 | Q96CX6 |
| Q02878 | Q53R41 | Q9NW81-4 | Q16760 | Q92835 | Q9NRG7-2 |
| Q8IWB7 | P30085 | Q9Y618 | Q9UPT8 | Q9NP81 | Q9HBD1 |
| Q9Y3Q3 | Q96G03 | Q1ED39 | Q8WU10 | P33316 | Q14155-5 |
| Q99633 | P27816-5 | Q7L804-2 | Q15058 | Q9H6A9 | P02545 |
| Q8IWA5-2 | Q5THR3 | Q9P2R7 | Q969E2 | P35236-3 | Q9NX08 |
| P19623 | E7EMN2 | Q96PZ0 | Q9BWT1-2 | Q00535 | Q9BVC5 |
| Q99741 | P52655 | O94888 | Q92973-2 | F8VW96 | Q96AD5 |
| Q9NWT6 | P48506 | Q9H098 | Q8NHP8 | O60502 | F8WCF6 |
| Q9P0P8 | Q13459 | Q13459-2 | P41091 | Q9UPP1 | P53611 |
| Q96LW7-2 | O43823 | P62491 | P50747 | Q9H2G9 | Q96SZ5 |
| Q9UHD8-2 | Q8TF30 | Q14137 | Q9NWW6 | O14925 | O75575 |
| O00559-2 | Q3L8U1 | Q96CT7 | O94925 | O14593 | O75469-7 |
| Q96FZ2 | Q15468-2 | Q06547 | P53801 | O14562 | P46778 |
| Q8IY18 | Q66K74 | E9PNP3 | O43427 | P78364 | Q9UBZ9 |
| Q99536 | Q9H9Y6-3 | Q06203 | Q2KHR3 | F6WH68 | A5YKK6 |
| P46779 | Q9UBC3 | Q13363 | Q9H6A0 | Q8IYL2 | Q9H6R0 |
| O75325 | Q13501 | Q96DH6 | Q8TCJ2 | Q9NX01 | O60331-3 |
| E7EQ69 | Q6P1L5 | Q13426 | Q6ZVH7 | Q9BUK0-4 | Q96GF1 |
| Q99728 | P78536 | Q9BQA1 | Q8IWT6 | Q8IWD4 | P62081 |
| Q8IVD9 | Q9HB07 | F8VQQ3 | P06280 | Q96L91 | P52292 |
| Q9BQF6-2 | Q969G3-2 | P33241 | Q9UKT4 | P09917-4 | O14893 |
| Q9Y490 | Q9BT73 | Q02241 | Q6IPU0 | Q9ULW3 | J3KNX4 |
| Q8N392 | Q9NPD8 | P41273 | O15498 | P26373 | Q155Q3 |
| Q8NB49 | Q9HD42 | Q9NW13 | P56537 | Q9UHB9 | Q9NUY8 |
| Q9NSI8 | B5MBW9 | Q9Y283 | P21953 | Q9HAD4 | Q8IZM8 |
| Q6UW56-3 | O43766 | Q8N9M1 | O15042 | O96019 | Q96T83 |
| Q13740 | Q92567 | Q92833 | P20340-2 | O75396 | Q9H9B1 |
| P61254 | Q7Z478 | Q13576 | Q9BTU6 | Q9H0C8 | B4DUT8 |
| P63241 | E7EVA0 | P62857 | Q96BP2 | O95865 | Q5T9C2 |
| Q8IYF3 | Q9H936 | Q53H12 | Q9ULX9 | Q9Y4X4 | O43156 |
| Q27J81 | Q9P265 | Q13905-3 | P36404 | Q6IQ32 | P20701-2 |
| O15118 | Q7Z7H8-2 | Q01459 | Q71F23 | Q8IYH5 | Q9H3H3-3 |
| Q15404 | Q8NF50 | Q15363 | Q96F86 | Q96TC7 | O75027-2 |
| Q8NBM8 | P10155 | P60953 | Q99470 | P46087-4 | B4DQP0 |
| Q69YI7 | Q9BS26 | Q9H0E9-2 | Q6ISB3 | P63218 | O75688-2 |
| Q7L9L4 | Q9NXN4 | Q6GYQ0-6 | Q14088 | Q9ULD2-3 | O43314 |
| Q8NCE0 | Q9NSK0-3 | P78346-2 | P08236 | P11387 | Q13889 |
| Q9BWU1 | P53609 | P46087-2 | P49137 | Q8TBF2-8 | Q9NPI7 |
| Q9NWV4 | Q6P4Q7 | O00170 | Q96T88-2 | Q9NZL9 | Q8WV60 |
| Q15119-2 | Q13309 | Q9ULZ2 | Q7Z4F1 | Q6DD87 | O43299 |
| P82663 | Q96G21 | O94905 | P62487 | P63151 | Q6ZW49 |
| P15848 | Q9NZJ4 | Q9Y2S7 | Q13425 | P62877 | Q86SG6 |
| G3XAB3 | Q13217 | Q96CN7 | Q96G28 | Q2NKX8 | Q5TAX3 |
| P20749 | Q96TA2 | Q4LE39 | Q6UXV4 | P42704 | Q9BQ52 |
| Q9NRA8-3 | O14672 | Q6VY07 | Q99704 | Q8N3L3 | Q15072 |
| Q68D91 | O95620 | Q68D85 | P55036 | Q13618 | Q8TC71 |
| O00512 | Q9UHJ6 | Q9UMS0 | P46776 | Q9NUT2 | O43189 |
| Q9UK33 | Q9HA47 | Q96BT3 | O95067 | Q96FS4 | O43314-2 |
| Q3MHD2-2 | Q9NXD2 | P04049-2 | Q49A26-5 | P62854 | Q9P015 |
| Q15056-2 | Q8WUH2 | Q8TAA3-5 | Q9NYW8 | Q9H992 | Q9ULM3 |
| Q15276 | P06744 | P80303-2 | Q86W42 | P08754 | Q9UIC8-2 |
| P09874 | P18846 | P40939 | P55957 | Q96IY1 | Q9NPG3 |
| Q9UKD1 | Q9ULW0 | P51948 | O94876 | Q4KMQ1 | O75947 |
| Q9GZY4 | Q96HN2 | Q8WVM0 | Q9NRF9 | Q9Y5J1 | Q9NTX5-5 |
| P84085 | Q05932 | Q9NQB0-3 | Q86V88 | P20340 | Q99961 |
| Q5TCQ9 | Q9H6R6 | P01892 | Q9BYD3 | Q93008 | O95551-2 |
| O95271 | Q13615 | Q9H0H5 | Q8N3A8 | P11274 | Q9NWZ8 |
| P26583 | Q6IPR3 | B4DLH4 | Q7Z6M4 | Q8N511 | Q86XL3 |
| O43681 | P62266 | Q6P1L8 | P50749 | C9JJN9 | Q6PGP7 |
| Q99590 | Q9HBH9 | O75643 | Q13572 | Q6BAA4 | P51003 |
| P35613 | Q9BRA0 | O95373 | Q13129 | Q9NVX2 | Q15061 |
| Q8WUK0 | Q9H8G2 | P18077 | Q86V85 | Q96Q11 | Q7L099-3 |
| Q9H4E7 | Q8TBZ3-2 | Q96CS3 | Q9H9J2 | Q96CN9 | Q9Y2D9 |
| Q9NRL2 | Q6ZN54 | Q8TEQ8 | Q9NX07 | P51812 | Q5JPI9 |
| Q13045 | Q9UN37 | O94832 | Q15031 | P62917 | Q9UH99-2 |
| Q07960 | Q08J23 | Q9NQZ6 | Q13029 | O95178 | O75608 |
| O14618 | P36873-2 | P25054 | Q96A35 | Q8TB52 | P14625 |
| J3KR72 | Q13724 | P41279 | Q9BQE9 | O95544-2 | Q9H492-2 |
| Q9ULD5-3 | Q92545 | Q7L1W4 | Q8WVR3 | Q15910-2 | Q15119 |
| Q14573 | Q9NPI1 | O00483 | Q96B54 | Q9ULZ3 | Q9UPN9-2 |
| O60674 | Q08117-2 | Q969P6 | Q02539 | Q9Y3Q8 | Q02083 |
| Q96HJ9 | P19784 | Q6UWP7 | P20700 | Q6WRI0 | Q96H35 |
| P78318 | Q12979-2 | Q6PCT2 | Q6NUM9-2 | Q5QJE6 | Q96FK6 |
| P51531 | Q9H910-2 | Q8IVH4 | P01591 | Q86XR7-2 | Q6UWU4 |
| Q3KRA6 | Q7LGA3 | Q15561 | Q6P2H3 | P16435 | Q2M1P5 |
| O75956 | Q8N3X1-2 | Q8N442 | Q02086 | P35606 | P49006 |
| Q86U70 | P15291 | P78362 | Q9ULL5-3 | Q8N264 | Q6P1K8 |
| Q8WUA7 | Q969R5 | P09917 | Q96F44 | Q10570 | Q9Y3D0 |
| Q3MIT2 | Q96EK9 | Q93009 | Q15397 | Q8NB78 | J3KPC8 |
| Q9HBM0 | O00507 | H0YMV8 | Q9BVQ7 | Q13243 | Q7L2Z9 |
| Q07812-2 | P23229 | Q96DH6-2 | Q15056 | P55211 | P57071-2 |
| P15927 | Q9H501 | Q99623 | Q9H5U6 | Q8WW43 | Q9BWN1 |
| Q9BQI3 | Q86Y38 | Q13557-12 | Q9NQW6 | Q15011 | Q9C0B0 |
| Q5VTB9 | F8WE65 | Q9BRJ2 | P04181 | 060828 | Q96EQ0 |
| P21796 | Q8WVK2 | Q13557 | Q9UBN7 | P19525 | O95155-4 |
| P55809 | Q9H582 | Q8WZ82 | Q96LD8 | Q9H081 | Q8WXH0-5 |
| Q9NQC7 | Q9H845 | Q9BYB4 | E7EQS8 | P24928 | P62937 |
| Q7Z2W9 | Q6P3W7 | Q6P158 | Q8IXB1 | Q53H47 | Q96GM5 |
| P61077-3 | B5MCU0 | Q5TKA1 | Q05D32 | Q9Y223-2 | P54136 |
| Q96BP3 | Q9NWS8 | Q86X10 | Q5TFE4 | Q8N8D1 | P68543 |
| E9PC47 | O00116 | P62937-2 | Q14980 | Q14980-2 | P10644 |
| Q9Y394 | P28749 | Q13535 | Q05086-3 | Q8TBB5 | O75688 |
| O95372 | O75251 | Q8NBU5 | P47974 | P62136 | Q96B45 |
| Q8N300 | J3KPT4 | Q9H9V9 | Q9NQ48 | O75676 | Q6P4H8 |
| J3KQ32 | O00463-3 | P42857 | Q9NQA5 | Q92878-2 | Q9NUN5 |
| Q8N5H7 | Q9BU70 | P30876 | Q96BY7 | Q9Y5Z4-2 | Q8WXD5 |
| O95139 | Q9NX38 | P43405 | Q7KZ85 | Q9UMS6-2 | Q9H5Q4 |
| O94761 | P62258 | Q9HCK8 | Q6UXH1-5 | P10768 | P05023-4 |
| P53567 | O95628-10 | O95059 | Q8NA72 | Q8NE01 | Q9UBK8 |
| Q14534 | Q9P000 | O14980 | Q99729-3 | Q9H6D7 | O14654 |
| O14734 | Q9BRT2 | P42785 | Q86Y79 | Q9H3M7 | P61225 |
| Q15723 | Q96SI9 | P20936 | Q13164 | Q6ZNA1 | P08631 |
| Q96RY5 | Q92536 | 060942 | O43189-2 | P52756 | P51649-2 |
| Q5MNZ6 | Q9Y5Z4 | Q13952-3 | Q6SJ93 | Q5SSJ5 | P26640 |
| E9PMM4 | Q8N8A6 | P30304 | P49748-3 | Q9HCG7 | P20339 |
| Q8WW35 | Q96GJ1 | Q9UGN5-2 | Q9H1B7 | Q14207 | Q9P0N9 |
| P42677 | Q8WZ42-12 | Q6IA69 | O14920 | P31483 | Q8N954 |
| Q9NUX5 | O14524 | Q5JSH3-2 | Q8TD17 | Q6ZRQ5 | O60888-2 |
| O94830 | Q96GA3 | Q7Z3K6-2 | Q5C9Z4 | A1KXE4 | Q68DN1 |
| Q9C0G0 | P22732 | Q9H2U2 | Q9BX63 | Q9BVP2 | Q9Y4P8 |
| Q13601 | Q9Y5T4 | Q9H714 | O00463-2 | Q15118 | O75461 |
| Q12792 | Q96T68 | O96018 | Q9H2C2 | Q9BZI7 | Q9HD34 |
| Q9H9G7 | Q96BR1 | Q5SVQ8 | P12235 | Q99497 | Q96BQ5 |
| Q9BYD2 | Q9H6Y7 | P42858 | Q9H061 | Q16630-2 | P20839-6 |
| Q96GD4-5 | Q6ZRP7 | Q96DV4 | Q5VWC4 | Q5XKK7 | Q01831 |
| Q9H3N1 | Q13415 | O75165 | Q01130 | G3V4K3 | Q9P1U0 |
| Q8ND04-2 | P40763 | Q16539-2 | P25098 | Q9BZ23 | Q9H0W9 |
| P12236 | Q10567 | Q9NVX0 | O60669 | Q9Y5B9 | Q9BXK1 |
| Q9UNW1 | Q8NA42 | P07741 | Q9UPN9 | P23458 | O60927 |
| C9JC87 | Q9UG63-2 | O75417 | E7EU96 | Q8WVS4 | Q13489 |
| Q8NI27 | P63000-2 | Q5T6F0 | P15153 | Q14185 | Q9NS00 |
| J3KNL6 | O00233 | O15156-2 | Q9NR50 | Q9Y303-3 | Q8NHZ8 |
| Q9NXG0-2 | Q8N0U8 | P61764-2 | J3KPX5 | Q14781 | Q14191 |
| Q86X52 | P36776 | Q15853 | O75643-2 | Q12824 | Q92841 |
| Q8N2G8 | Q9Y448 | Q5JSJ4 | Q8IYB7 | Q96HE9 | Q8N7M2 |
| Q8IWT3 | Q12904 | Q4ZIN3 | Q8WUM0 | Q9UNZ5 | Q8NBT2 |
| Q9UGP8 | O00541 | Q9GZR2 | Q8WXA9-2 | O95881 | Q86YH2 |
| Q9Y399 | Q96AE4-2 | O75880 | P54098 | Q9NVH0 | P43250-2 |
| Q86VU5 | Q9GZT6 | Q9NVM4 | Q9BRA2 | P17706 | Q14596 |
| Q9C0F1 | E7EWW0 | Q96RT8-2 | Q4AE62 | E9PQA6 | Q9Y2C4 |
| P78316 | Q6P1N9 | Q9HCM4 | Q9Y314 | Q9H0B6 | Q9BU02 |
| Q9UPU5 | Q15542 | O14867 | P47914 | Q6PIF6 | Q4G148 |
| P38646 | Q96EK5 | Q6ZUV0 | P51826-2 | Q9H446 | P35241 |
| P17039-2 | P27797 | Q7Z401-2 | Q9BRJ7 | Q15021 | Q10567-3 |
| Q8NDV7 | Q7L211 | Q9Y2Z4 | Q9UNF0 | Q8NA19 | Q99618 |
| Q7KZF4 | Q9Y388 | Q9HAW4 | Q9P0I2 | Q8N201 | Q7Z6J9 |
| F6XY72 | Q9HC36 | Q9UIQ6 | Q8TCY9 | P51659 | Q9UNZ2 |
| Q9UM82 | Q07021 | P36542 | P31947 | P09543 | Q96RR1 |
| Q9H1C4 | 043309 | O15121 | P36405 | P62158 | Q15691 |
| O75391 | P08559-4 | Q96NC0 | Q8TF05 | Q96GM8 | O14678 |
| Q9NV06 | Q96AH0 | Q96FI4 | Q9HA64 | Q8N7R7 | Q8TCT0 |
| Q9Y487 | K7EM18 | Q9Y421 | Q969Q5 | K7ELL7 | P20042 |
| Q7Z434 | Q9Y2M0 | Q96L94 | C9JLB7 | Q8N3C0 | P49454 |
| Q9BSV6 | P21675-4 | Q9UBB9 | J3KNF5 | P09661 | Q9P275-2 |
| Q96GQ7 | O15229 | Q9Y6D6 | O14579 | P63220 | Q49A26 |
| Q9Y3A4 | P48551-2 | Q5TA45 | Q8TEY7 | Q93034 | O75153 |
| O60281 | P63092-4 | Q9P021 | P54577 | Q13162 | 095470 |
| G3V1L9 | Q6NUM9 | P01834 | Q96BR6 | P48553 | Q96N11 |
| O00635 | P27987 | E5KLJ5 | Q9Y4X5 | Q06609 | P32121-3 |
| Q9BZL4 | P53618 | Q92900 | P30050 | O75190-3 | P47897 |
| Q53G59-2 | Q6XUX3 | O60870 | Q9UNX3 | Q15392 | P13639 |
| P61106 | B0AZT5 | P49406 | Q16540 | Q96JB5 | P55145 |
| Q9Y4C4 | Q9P2W1 | Q709C8 | O94916-5 | Q9NVR5 | Q9BZH6 |
| Q4G0P3 | Q96QE5 | Q5JPF3 | Q9UBS0 | Q96HW7 | Q9BRQ8 |
| Q9Y4R8 | P28062 | Q8IY17-4 | Q9Y3I0 | P54277 | Q96DZ1 |
| Q5HYK3 | O15550 | Q9UIF7-3 | Q9HCJ0-2 | Q9P2P6 | Q13163-2 |
| O43159 | Q15776 | Q6P2D0 | Q6NSJ0 | O43361 | O95169 |
| P15907 | O43402 | Q96BH1 | O00743 | F8W1B3 | O76031 |
| O60759 | Q92623 | P10515 | Q8WXX0 | O14646 | P42766 |
| Q8NCA5 | Q9NP92 | Q6IQ21 | Q15361 | Q9Y6N1 | Q8NHU6 |
| P78345 | Q7Z3B4 | Q96EW2 | Q5JUW8 | Q68DI1 | P54727 |
| Q9UBW8 | P02794 | B4DWF2 | Q9ULF5 | C9JA08 | Q8N1B4 |
| O60282 | P29372-4 | P78560 | Q96FV9 | Q9NXR8 | Q7RTN6 |
| E9PKB1 | Q9Y5K6 | Q99943 | P23919 | Q9Y281 | Q9C0G6 |
| Q63HQ0 | Q9UL40 | P84550-2 | Q9NPF4 | Q9HCD5 | Q9UL40-2 |
| E5RIM7 | Q9BVS5 | Q8N5Y8-3 | Q9NXW2-2 | O15357 | Q6XQN6 |
| Q8IWT0 | Q8ND76 | O75352 | O43670-4 | Q9HCG8 | Q8WXQ4 |
| Q8IYU8 | Q3ZCT1 | P61224 | P07954-2 | Q13315 | P0DI81 |
| Q14934-3 | Q8TD30 | Q15904 | O43175 | Q13214 | P52747-3 |
| O94925-3 | Q13823 | P61964 | Q5VZE5 | P49840 | Q9BU64 |
| Q14146 | Q14376 | A0AVT1 | P18433-3 | P46108-2 | Q8WUQ7-2 |
| Q9NS93 | O95551 | O75373 | Q86WH2 | Q96AE4 | F8W727 |
| Q9Y664 | P42285 | Q8IXH6 | Q8N5N7 | Q9BTC0 | Q8N4V1-2 |
| Q9BQP7 | Q86US8 | Q9H0E2 | Q969T9 | Q8N720-3 | Q8NE09 |
| O75436 | O76071 | P49790 | P22102 | H0Y2M6 | O15162 |
| P78332 | Q8NCK3 | O95782 | Q8WZA9 | C9JLR9 | Q14258 |
| P26639 | Q9NVN8 | G3V1S3 | B4DU55 | Q9Y305-4 | Q13488 |
| Q9P2D1 | Q6Y288 | P41002 | Q8IXM7 | Q99676 | Q9Y485 |
| Q96H55 | Q9UNS1 | Q9Y3D5 | Q12769 | O00567 | F5GYQ1 |
| Q92805 | Q9BRD0 | Q15398-3 | Q8TDQ7 | Q53GS9 | Q9UHR6 |
| P14735 | Q86XK2-5 | Q9UPY3 | Q3KRA9-2 | Q9P2N6-3 | O43913 |
| Q9NTZ6 | Q15291 | Q9UGC7 | Q8WVP5 | Q69YL0 | Q96JC1-2 |
| Q8N488 | P16871 | Q14152 | P52788 | Q86XN7 | Q5T1M5 |
| Q96GG9 | Q7L1T6 | O75387 | P54802 | Q32P51 | Q2M2I8 |
| Q9NVS2 | Q9UNN5 | Q9ULT8 | Q07889 | Q3ZCX4 | Q7Z6L1-2 |
| Q8IWV2 | Q15477 | H3BM74 | Q8WUH6 | Q8N6N3 | Q99614 |
| P31785 | Q92696 | Q9BYG5 | O00462 | P17026 | Q8TDG4 |
| Q15651 | Q8IZ07 | Q96S15 | Q14790-6 | P68402 | Q9Y237-2 |
| Q86X95 | Q7Z412 | Q92889 | Q13542 | P35240-2 | P35637 |
| O75312 | D6REX3 | P51957 | Q9H9B1-4 | Q9BY50 | Q96CM8-2 |
| Q6DKI7 | Q9NV56 | Q96IW7 | Q9UBC1 | Q8NB50 | Q08379 |
| P62341 | F5H156 | Q7L2E3-2 | Q9NYK6 | Q9NWD8 | Q14978-2 |
| Q16537 | P16401 | Q9BRJ6 | Q3B7J2 | Q9H993 | P62995 |
| Q8IXH8 | Q9BPX7-2 | D4PHA4 | Q96NW7 | F5H604 | Q8N1G4 |
| O96020 | Q96SK2 | Q8WUU4 | Q3KP31 | P55769 | O00461 |
| P49590 | O14681 | Q99836-6 | P83876 | Q8IX18 | P29728-2 |
| O96005 | B4DN88 | O43707 | Q14653 | O15211 | P41252 |
| P39023 | P51580 | Q9UK76 | Q13435 | O75223 | P15622-3 |
| Q6ZUT1-2 | Q8N5P1 | Q9BTV7 | Q8IZ73 | Q5T5Y3-3 | O75925 |
| P20248 | Q9ULI0 | Q92845 | O95674 | Q86YB8 | Q96NB2 |
| O43824 | Q15629 | Q5W026 | Q14166 | Q13098-7 | O14662-2 |
| F5H0B0 | Q9BUJ2 | Q03936 | F5GXV7 | Q9ULX6 | Q96K21 |
| Q92922 | O75821 | Q7L590 | Q14562 | Q9NPF5 | Q86SF2 |
| F5GXE4 | P06396-2 | Q9HAP2 | Q6IAA8 | Q96S55 | Q9NXW9 |
| Q9H1Y0 | Q16670 | Q9BSK1 | Q5VWQ8 | P28715 | Q13569 |
| P31689 | Q8N0W3-2 | Q13404 | G3V2F7 | Q9H2U1 | Q6PDB4 |
| Q9BWD1 | H7BY35 | B3KSJ7 | Q69YN2 | Q9Y316 | Q96GW9 |
| O00139-2 | Q6ZNJ1 | O95071 | Q96L73 | Q7L5D6 | Q8IYE1 |
| O75448 | Q9NWB6 | Q13625-3 | P25963 | Q06330 | Q8TAX9 |
| Q9HAB8 | Q9UPN7 | P55210 | Q96S19 | O15355 | Q6UB35 |
| O43734 | Q5JRX3-2 | Q9Y6D5 | Q99627 | Q9UIM3 | P30047 |
| Q6IEG0 | Q96B36 | Q96P16 | P23109 | O15440 | Q7L099-2 |
| Q5T7W7 | A4D1P6 | O43439-4 | Q9BVI0 | P08962 | Q96DA6 |
| P40259-3 | Q9HC21 | O14746 | O60292 | Q13409-2 | Q14141 |
| Q13137-4 | M0QY77 | Q8N859 | Q9BXA9 | Q9BYW2 | Q86UQ4-4 |
| Q9P0J0-2 | Q86XP3 | Q9NZ01 | P63092-2 | Q9NSE4 | Q96ME7 |
| Q8N5C6 | Q9NV35 | Q9P0L1 | Q8IZD2 | Q6P6B7 | Q96R06 |
| Q9Y3A5 | Q8IWX8 | E3W974 | Q8IWR0 | P30049 | Q96SB3 |
| O75822 | Q96F07-2 | P43243 | Q53HL2 | Q00059 | P0CW22 |
| P84243 | Q96C34 | Q9BX10 | Q9NVH2 | O75140-8 | Q4AC94 |
| Q9HAF1-3 | Q7Z6M2 | A0A044PY82 | P62834 | Q7Z6W7 | Q8IXJ9 |
| Q9Y608 | Q8IX90 | D6RAR4 | O75995 | Q86XF0 | Q9UBT7 |
| P09651-2 | Q9BUL8 | P48147 | Q8WXA3 | Q99467 | Q9Y3Y2-3 |
| Q8WV16 | Q92523 | Q7L592 | Q9Y241 | P50993 | Q9H4A6 |
| P23497-4 | Q96I76 | Q7Z6V5 | Q99674-5 | A8MXP9 | O94875-4 |
| Q8NCY6 | O75037 | Q13206 | O14981 | Q7Z6J8 | Q96PU4 |
| Q8N8R7 | P50416 | Q96BD5-3 | Q8TBK6 | P35269 | Q8IWA4 |
| Q8IY31-2 | Q5FWF5 | P53794 | Q8TF50 | Q9Y450 | Q14978-3 |
| P57678 | Q9UBB5 | Q15046 | Q96E29 | Q6P1X5 | Q7Z6I8 |
| P12955 | Q6NUK4 | Q38SD2 | D3DWI5 | Q9C037 | Q5T1M5-2 |
| Q0VDG4 | Q9H1A4 | Q9NQZ5 | Q8IYI6 | Q9HCR9 | Q96F07 |
| Q9Y4U1 | Q7Z4I7-2 | A0A075B6F3 | Q96SE7-2 | Q92546 | P54652 |
| Q99638 | J3KQV8 | Q70CQ2 | P21127-2 | Q5T6N4 | P17066 |
| Q6UVJ0 | O95714 | O75190-2 | Q5UIP0 | Q71F56 | Q7LBC6 |
| Q96GS4 | Q969X6 | Q9NYK5 | O95864 | Q6IQ22 | Q01968 |
| Q6FI81 | Q96FH0 | O75390 | P40937 | Q16740 | Q9Y6H1 |
| Q9H6Y2 | Q86WB0 | Q9H9B4 | Q9UIF9 | Q9UBU6 | Q5S007 |
| P16455 | P00488 | Q86UV5 | Q8IXI2-3 | O95429 | Q92619 |
| E9PKP7 | Q96PQ7-3 | O75937 | Q86WA6 | Q8IWW6 | O75629 |
| Q07912-3 | Q99613 | P41240 | Q99766 | Q49AG3 | Q9BRQ0 |
| P62841 | Q5T2R2 | Q9Y5X3 | Q99952 | Q53QZ3 | Q8WV07 |
| Q8TAD4 | Q96EY9 | Q9BRX9 | Q92990 | Q9Y678 | Q15643 |
| Q9Y484 | Q9P2S5 | Q8NAA4 | Q7Z7L9 | H0YK93 | Q6PK18 |
| A6NMQ1 | Q9NYL9 | Q5JTD0-2 | Q9BYD6 | O75955 | Q9UBS4 |
| Q92609-2 | P14317 | Q86UW9 | O43255 | O75616 | Q02223 |
| P28482 | P46108 | P49448 | O14492 | Q9BTF0 | P45985-2 |
| P53990-4 | Q13232 | P06239 | P34896 | P30566 | O43493 |
| Q8N3Z6 | P47224 | P07814 | Q9H6S0 | P49005 | P60228 |
| O15116 | Q9NQS7 | Q9P2E5 | Q9H2K8 | Q15366-4 | Q7Z2E3 |
| Q8TC12 | Q93073 | Q8NFC6 | P63092-3 | P84098 | P17948 |
| Q13907-2 | Q9BZV1 | O75528 | Q99549 | Q8TE02-4 | Q8WUF8 |
| Q96J42 | Q07864 | F5GX51 | Q13588 | P35754 | Q8IXM2-3 |
| P35789 | E9PF18 | Q15366-5 | F8VZX2 | Q9Y4E5 | Q9NVE7 |
| Q8NGR9 | Q5T6C5 | Q8IYT2 | P14174 | Q7Z7L7 | O00232 |
| O95400 | Q9UHW5-2 | Q8WXI4 | Q8IVF7 | Q4V328 | O95397 |
| Q96CB9 | Q9P219 | P53990 | Q5T1C6 | P17980 | Q8TC56 |
| E9PC69 | Q9UEE9 | Q9BRX2 | Q9Y3U8 | O00488 | P16989 |
| Q9H270 | Q9HC62 | Q92499 | P83731 | P24539 | Q06547-3 |
| Q13277 | Q8IWI9-4 | P10412 | P34932 | Q86Y82 | Q96NB3 |
| A6ND36 | P56134 | O43829 | P37198 | Q15366-2 | Q09028-2 |
| D6RH02 | O00264 | Q9P2B7 | E9PDF5 | Q8TC07-2 | Q9UDY4 |
| P50750-2 | P78352-2 | O43147-2 | Q9Y3C4 | Q9BSJ2-4 | Q9NPA0 |
| Q06124-2 | O14775 | Q13564 | Q96IZ7 | Q969S8 | Q8WW12 |
| Q6UWB1 | P30519 | Q9NUQ2 | Q15937 | Q92930 | Q4VC44-3 |
| Q5JTZ9 | P04350 | Q9UKK9 | O95081 | Q15311 | Q96CP2 |
| O43303 | P84103 | R4GNH3 | Q6ZU80 | Q15393 | A8MW78 |
| Q7LBR1 | O75170-5 | P46777 | Q15785 | Q8IY22 | P33991 |
| O75157 | O43808 | Q5TC12 | P83369 | Q96BT7-4 | Q6UB98 |
| Q9HBM6 | Q8IY17-5 | Q9H814 | A8MT69 | O75431 | Q96DT5 |
| O60832 | Q99571-2 | P09429 | Q96CW5 | Q9Y2S0 | Q96C36 |
| O43172 | Q6NZY4 | Q8NF64 | Q12959-2 | Q8NEP9 | Q6PIJ6 |
| Q9NPQ8 | P57737 | B9EGE7 | Q13951 | O43747-2 | Q9Y6D0 |
| Q9UIS9-11 | P25398 | O14787-2 | P27635 | P63167 | Q96HA8 |
| Q9BW85 | Q9UKX2-2 | Q9H1E3 | Q6KC79 | Q8N587 | Q5VYM1 |
| Q8IYB8 | Q7L014 | P49247 | Q8N9V3 | P16150 | Q9P1Z2 |
| Q5T6V5 | F8W8F9 | P48454-3 | P30305-4 | Q96EA4 | Q15628 |
| P02686 | Q9UKB1-3 | P49721 | P52739 | C9JYJ0 | Q7Z3B0 |
| Q96J02 | P22307 | O15371 | Q3KQU3 | J3KPE3 | Q96RP9 |
| Q8IYR2 | O15126 | Q9BQ15 | Q8TDR0 | A6NGJ0 | Q9BRX5 |
| O95477 | P20226 | J3KQ12 | Q96P70 | P55735 | Q9NXF7 |
| Q8WYA6 | Q6UWE0 | Q9UL46 | P23511 | E9PAV3 | Q6DCA0 |
| Q8NI60 | Q8IZ21-2 | Q8N5M1 | Q9H8E8 | P40227 | P48380 |
| Q86V48 | Q14997 | P49116-2 | Q4FZB7 | Q6ZMS4 | Q9Y679 |
| Q5SWX8 | Q86V15 | Q9C0J1 | Q3KQZ1 | Q9Y3B8-3 | P07992 |
| P62906 | Q8NDA8 | Q00613 | Q5VWG9 | P51116 | Q9Y2B0 |
| Q7Z2Z2 | Q9BX70 | Q8WUM9 | Q09028 | Q8NBN3 | Q8TB72-3 |
| C9JFV4 | O75694 | O94875-11 | Q16667 | Q9Y6K0 | Q15366 |
| Q9NWT1 | Q8TDH9 | P61086 | Q9UHA3 | Q96BN2 | P54132 |
| P11277-2 | Q9Y4F9-2 | Q15751 | Q86VX2 | Q9BW61 | Q9Y2I7 |
| Q9Y4W2 | P61160 | H3BQQ2 | J9JIE6 | Q8N122 | Q8IYB5-3 |
| P11766 | Q969Q4 | Q9Y4L1 | Q99622 | Q96RQ3 | Q92539 |
| Q9H0G5 | Q03519 | P11586 | Q8NC51-2 | Q8IZH2 | Q9UIS9-9 |
| Q6IBW4-4 | Q99805 | Q8WXX5 | Q8N1F7 | Q8WYQ5 | Q7Z4G4 |
| Q9Y3B8 | Q9BWH6 | Q86UK7 | Q9P2P5 | Q92896-2 | Q9BXL8 |
| P09651 | Q9P2E3 | Q92625 | Q96E14 | O94763 | O95989 |
| Q4KMQ2-2 | Q8WTS1 | Q9BXS5 | Q92504 | Q8WVY7 | Q13049 |
| Q15642 | Q7L4I2 | Q96AQ8 | P55327-4 | Q9H1K1 | Q96MD7-1 |
| Q5HYJ3 | O75843 | O15297 | Q6PI47 | Q15843 | P61247 |
| Q96EZ8-2 | Q12849 | P52701 | Q9UBD5-2 | Q8IZ81 | P83111 |
| O60508 | O15212 | P0C7T5 | P49770 | Q9H875 | Q8N8A2 |
| P49841-2 | Q96I24 | O14730 | Q9H9Y2 | O14628 | Q14119 |
| O94952 | P53671-3 | J3KPF3 | Q96KP1 | Q6W2J9 | Q9Y2R2 |
| Q96C90 | Q8NFA0 | Q96LI5 | Q9UHY1 | O43181 | O00628 |
| P59923 | O95625 | Q9ULD4 | Q8NG11 | Q9Y324 | Q9UKT7 |
| Q6I9Y2 | J3KQY1 | Q8N3Y1 | Q9BRU9 | B1AN89 | P62263 |
| Q96BW1 | O75362 | Q9Y2K1 | Q15365 | Q9NX40 | Q9UKL3 |
| Q86UY6 | Q5SY80 | Q8N141 | Q9HB20 | Q9GZN1 | Q8TBZ6 |
| P19447 | Q9H2H8 | M0QZW1 | Q8NHG7 | Q6UN15 | Q9UJX2 |
| O95292 | Q9Y478 | Q9Y5Y5-2 | P52746 | O60921 | O60841 |
| Q16626 | Q86Y37 | Q92552-2 | Q9BYE7 | Q9NYP3 | P21281 |
| Q8WWI5 | P33176 | Q9UKN8 | Q6UB99 | O75420 | P17040 |
| Q9ULU8-4 | P05141 | G3V599 | P08047 | Q9Y2Z0-2 | P46940 |
| J3KNN3 | Q7Z5R6 | J3KNB2 | O75477 | Q16512 | Q16656-4 |
| Q96FF9 | Q13510-2 | O00139-4 | F8W8F5 | Q5JVG8 | P16989-2 |
| Q8N972 | Q96BU1 | Q9NVH1 | Q9UQ88-2 | M0R0E0 | Q9NNW5 |
| Q9UHY8-2 | Q9NYC9 | Q86V81 | Q96ND0 | P19838-2 | Q5T7B8 |
| Q14558-2 | Q8N137-3 | Q659A1 | Q9P2D7 | Q13439 | Q8TDN6 |
| Q9Y3B1 | Q5SRE5 | Q9NYH9 | Q8NB46 | Q8WTW4 | Q13769 |
| Q9BTT4 | Q6PIK3 | O94842 | Q7Z4L5 | O43617 | P05387 |
| P46100 | P17480 | Q4VC05-2 | Q5XPI4 | Q49AM3 | P13807 |
| O00308 | Q01094 | Q9NXG2 | Q5JVF3 | Q13347 | Q7Z3U7-5 |
| O43316 | Q9H3E2 | P54886 | P61586 | Q99471 | O75319 |
| Q9NQP4 | Q6P1Q9 | Q15814 | Q92802 | P46019 | P86790 |
| Q5VSL9 | Q9NZJ5 | O75123 | Q9BVC6 | P78406 | O15360 |
| O75182-2 | Q12789 | Q9H269 | Q96D53 | Q9H9D4 | Q8IZD9 |
| P78371 | Q8NFG4 | Q15653 | Q2T9J9 | P0C671 | Q16850 |
| P54886-2 | P30154 | Q5T011 | Q86TB9-4 | Q9UK97 | P46527 |
| Q14188 | O95684-2 | P15531-2 | P11908 | Q147X3 | Q96HA1 |
| Q15054 | Q15700-2 | Q96DU7 | P62942 | Q8TF46 | O95453 |
| Q9NQV6-6 | Q8NHY6 | Q9Y4K3 | Q9Y4E6 | Q2NL82 | P15884 |
| Q8WUD6 | Q9Y383 | Q5T447 | Q14151 | P22234 | Q9Y5L0 |
| Q9UEG4 | Q9Y2X0 | Q6NVH7 | Q5QJ74 | P60604 | Q9Y2Z0 |
| Q9H6H4 | Q9UHD8-4 | Q9BUF5 | A8MWQ5 | Q9Y371 | Q53F19 |
| Q99733-2 | P68371 | Q13630 | Q9H2Y7 | Q5RIA9 | Q99873-3 |
| O60476 | H3BQK9 | P49354 | Q9UPQ9 | Q9HAU5 | Q9BYM8 |
| Q5VU65 | Q15633 | Q15746 | Q8NDZ4 | Q9NWZ5 | Q9BXP5-3 |
| F8VVA7 | Q9BPZ3 | O00459 | Q9H9S4 | H3BNR1 | Q9P258 |
| P33993 | Q96QG7 | Q6IE81 | Q07666 | Q9NYU1 | O95758-5 |
| P46976 | Q15833 | Q9BZE2 | Q15526 | O95573 | Q96KQ4 |
| Q8IUI8 | Q17R98 | Q2TBE0 | P08238 | Q8IXQ6-2 | Q9UKD2 |
| P22090 | Q9UEU0 | Q7Z589-7 | Q6ZT12-4 | Q8IVL0 | P42574 |
| Q08378 | Q9UQ84 | Q9HCU5 | Q6ZU65 | Q3MII6 | Q7L775 |
| Q5T280 | Q969E8 | Q99942 | Q8WWC4 | Q9UHQ9 | Q9NP50 |
| Q92997 | Q15173 | Q14669-2 | P55197 | P43034 | Q9UMR5-3 |
| Q92572 | Q9NY27 | F8W950 | Q8WYL5 | Q9UMN6 | Q9C0E8-4 |
| Q92564-3 | Q15120-2 | P28065 | Q92604 | P24534 | A6NDY0-2 |
| Q6PH81 | Q14966 | P62714 | Q96A26 | Q9NUM4 | Q96G25-2 |
| Q92530 | Q03468 | Q68DK7 | Q7Z570 | Q5SVZ6 | Q7L5Y9-2 |
| Q66PJ3-3 | Q96JJ7 | Q9NQG7-4 | F8VXU5 | Q9NRK6 | Q16795 |
| Q99661 | O60231 | Q9NV72 | Q8IZP0 | Q6ULP2 | G3V4H1 |
| P04899 | Q5H9R7-5 | P45983 | Q8NEE6 | Q8TD31-2 | Q8IYB3-2 |
| F5H527 | O60610 | Q6IBS0 | Q9H4B7 | Q9BU61 | Q7Z7H5 |
| Q13033-2 | Q5T0F9 | Q9UL25 | O95229 | P11171 | Q9Y508 |
| Q96EK4 | P49815 | H0Y413 | Q03923 | Q8TCB7 | Q6PKG0 |
| Q92673 | Q9NUQ7 | Q99418 | P11802 | P53582 | Q14691 |
| Q03701 | Q9H9A7 | Q8TED0 | Q99873-4 | P59998-2 | Q96K31 |
| Q9C0J8 | P60891 | P27448-3 | P27448 | Q9UPN3 | Q15036 |
| Q53GI3 | Q8TA86 | O15504 | P56385 | Q9HBH5 | Q6AZW8 |
| Q8NCE2 | P53041 | Q96IV0 | Q86Y13 | Q9Y4Y9 | Q8TD16-2 |
| A8MTY0 | Q49AR2 | O43572 | Q96DY7 | Q9BRP8 | Q8WVD3 |
| Q8WUF5 | Q92614 | Q66PJ3-2 | Q66PJ3 | Q9BRP1 | Q14498-2 |
| O15347 | P40222 | Q9UKY1 | O43143 | Q15834 | Q9UNX4 |
| A6NP11 | Q92614-5 | Q68CQ7 | Q53TQ3-2 | O60763 | Q9Y4D1 |
| P28066 | O96007 | Q15131 | O15394 | Q12901-2 | P14618 |
| P19474 | Q9P0W2 | O00257 | Q14697 | Q5W0Z9 | Q12913 |
| Q8IXI1 | P46379-3 | A0A075B6T4 | P24666-2 | Q9BSW2-2 | Q13123 |
| Q99735 | Q5JPI3 | O14974-3 | Q9H1I8 | P18669 | P57768 |
| Q8NEZ2 | P49643 | P30414 | Q8WU67 | Q96GE4 | Q14CZ7 |
| Q9Y2S0-2 | Q9Y285 | Q92688 | P30046 | Q8NDF8-5 | Q96EF0 |
| Q8N573-2 | Q6NW34 | P51809 | Q6PD62 | P17014 | Q9Y5T5 |
| Q13131 | Q9NYT6 | Q13509 | Q9NR31 | Q9UPS6 | Q96M89 |
| Q9H2G4 | Q9HC44 | O14974-4 | P56715 | Q8TBE9 | Q15042 |
| P21817 | Q8NHV4-3 | Q10469 | Q06587 | Q16533 | P43246 |
| E5RIN2 | O14879 | O15258 | P13984 | Q86SQ7 | O60506-3 |
| Q9NXK8 | Q9Y6B7 | P24298 | Q5TDH0 | P25205 | Q92995 |
| P16403 | Q99714 | Q6ZNG1 | Q9BVT8 | P39687 | P78347 |
| P49642 | Q9NZ32 | Q7Z3J3 | Q9NR30 | P18583-3 | Q9NQT5 |
| Q9NPD3 | Q8IX12 | P26368 | Q15569 | Q9NR82-6 | Q9ULC4 |
| P22033 | Q8NEZ3 | Q5FWF4 | Q96PQ6 | Q9H147 | Q5M7Z0 |
| Q96IR2 | O43166 | Q9NVJ2 | B8ZZD4 | Q9NXC5 | P43686 |
| Q66K89 | Q9H9T3 | P82979 | E7ESW6 | Q86VF2-5 | P62826 |
| Q9P1Y6 | P50151 | P04004 | Q8WVT3 | Q96KM6 | Q8N4J0 |
| Q08AG7 | P61966 | Q9BW27 | Q66PJ3-7 | Q96BM9 | Q14156 |
| Q5SXM2 | P51148 | Q9HBE1-4 | Q96S38 | O75820 | Q96ST2 |
| Q9P2I0 | P49321 | Q8IXW5 | O60765 | P11172 | Q6WCQ1-2 |
| Q86X83 | Q9BXJ8 | Q9NQR1 | Q9GZR1 | Q96BX8 | Q96C86 |
| Q6GMV2 | Q9H8Y8 | Q2TAY7 | Q9H2K0 | Q96LW1 | Q9BT40 |
| Q96JK2 | X6R3V9 | Q9UK73 | Q9Y3A6 | Q13555-8 | O95758-6 |
| P52429 | Q9UJX6 | Q8WVZ9 | Q86UE3 | Q96RF0 | Q9ULJ7 |
| P18583-6 | Q9HCL3 | Q5BKY9 | Q8NCG7 | Q9Y4E8 | Q96PB7-6 |
| Q14241 | Q8TF01 | Q9UL33 | Q9H8K7 | Q9H7D7 | P62269 |
| P62899 | Q9UGR2 | Q9UNY4 | O75865-2 | Q6ZV29-5 | P18754-2 |
| Q9HD47 | O43865 | P24666 | P61353 | Q9BUE0 | P00338 |
| A6NK75 | Q9H7B4 | Q6ZUX7 | O14974 | Q8N3Z3 | Q8IXQ5 |
| Q9ULH1-2 | P11912 | P49916 | P10746 | Q8NI77 | Q01167 |
| O60318 | Q9UER7 | Q6PIY7 | A6NHR9 | Q9P032 | O95757 |
| G3V1V4 | Q8WXF1 | Q13177 | Q96BW5 | P36578 | Q7L5Y9 |
| Q8IVL6 | O94892 | Q15545 | P49588 | Q9H871 | Q8IUI8-2 |
| B7Z4W5 | Q6P587-3 | Q9NUQ8 | Q8TCD5 | Q6PCD5 | P46934-4 |
| Q9H741 | Q9UL58 | P49750-4 | P57679 | Q99436 | Q9Y3X0 |
| Q8TAV0 | P48643 | Q9H6H4-2 | Q9BSM1 | Q92698 | P20073 |
| Q8TF76 | Q9NQZ2 | Q99575 | Q8TEX9-2 | Q56P03 | P47813 |
| A8MQ14 | Q9BXF3 | P55060 | P61513 | Q02078-6 | Q9ULD8 |
| Q9NZZ3 | O15234 | Q5VWQ0 | Q8IWA0 | D6RAR6 | P04920 |
| Q9NUJ1 | Q96T58 | Q99570 | Q9Y3S2 | E5RJD8 | Q96B23-2 |
| Q09FC8-5 | Q86TJ2 | Q6ICB0 | P61758 | P20585 | 094992 |
| Q9H467 | Q9Y255 | Q14697-2 | Q7Z4H7 | P62244 | Q96PV6-2 |
| P0C024 | P43007 | Q9NRH1 | P62249 | Q96M27 | O95777 |
| Q9Y4C1 | Q9UHR4 | P67775 | Q8WXW3 | Q92600-2 | Q12894 |
| Q9NNX1 | O14713 | Q5VWN6 | O95163 | Q9NSV4 | Q96RE7 |
| P11166 | Q92985-4 | F5GX68 | Q15428 | P23258 | Q9NZJ0 |
| O75882 | Q92994 | Q96PU5-7 | P0CG48 | Q86Y01 | Q9UBI1 |
| P07305 | Q99490 | P60709 | P20645 | Q99832 | Q3T906 |
| P49756 | Q13111 | O60566-3 | Q9ULQ1-3 | A8K0S8 | Q8IV32 |
| O94763-4 | O00203 | Q01469 | Q8N0Z8 | Q969U7 | F8W9L8 |
| Q9UNP9 | P21964 | Q9HAU4 | Q9UNT1-3 | Q9Y6K9-2 | Q32P41 |
| Q9BWJ5 | Q9NYY8 | Q9H9F9 | Q14BN4 | P04222 | Q13445 |
| Q9NQY0 | Q9UPN4 | Q6ZN08 | Q9HAW0 | P49755 | P24386 |
| P67809 | O60934 | P62847 | Q9H074 | Q7Z4H3 | Q9UDW3 |
| Q9NPJ6 | Q7L0Y3 | Q8WUI4-5 | Q12980 | Q7Z7F0 | P10074 |
| Q9BXI6-2 | Q9NP79 | Q9UDW1 | P02647 | Q86T82 | P08922 |
| Q9UHB4-2 | D3YTA9 | Q15652 | Q9NUP7 | O75467 | O14641 |
| Q9Y570-4 | Q15286 | P49207 | Q14974 | Q96EB1 | Q9P2T1-2 |
| P80723 | P08473 | Q8IVP9 | Q9NRN7 | Q9UL01 | Q02224 |
| Q92613 | Q5JU67 | M0QZI3 | P54646 | Q9Y266 | Q5TB80 |
| D3YTG3 | E9PRY8 | Q9NQC3 | O00370 | O43390 | Q96EE3-1 |
| O00442-2 | Q6AW86 | Q9NWV8 | P17081 | P51665 | P78537 |
| P0CG39 | Q8NEY8-8 | Q9Y5Q9 | Q14746 | Q96HY7 | O60547 |
| P18615 | Q13155 | P57740 | Q9UH62 | Q9P0M6 | P04406 |
| O15047 | P51991 | X6R7X0 | O15530 | Q6ZS11 | Q5VVQ6 |
| Q8N4Q1 | H3BS01 | Q12872-2 | Q9Y6R9 | Q86WN1 | Q9ULX3 |
| P09497-2 | B8ZZ87 | Q9P287 | Q16186 | Q8TAP6 | Q9HCE5 |
| Q9BVW5 | Q16181-2 | Q8N5B7 | Q96T37 | A1L188 | P58317 |
| Q9Y3I1 | Q6RFH5 | O60784-2 | Q8N5R6-2 | Q9Y2Y1 | Q6PHR2 |
| O75348 | Q8TD26 | Q7L8L6 | Q9UJU6 | F6S8M0 | Q16181 |
| O15511 | Q8WUX9 | Q13077 | Q12981 | O43861 | Q8NEY8-9 |
| E9PDK2 | P32298 | Q9NPA5 | Q9NT62 | Q9UI30 | P26358-2 |
| Q7L3T8 | P49591 | Q9NXR7 | O43293 | Q92620 | Q9NVP1 |
| M0R2Z9 | Q9NPA8 | Q6P2D8 | Q8NE71 | Q9UEE5 | P51398 |
| Q7Z3D6-2 | P42568 | Q68CR1 | Q8NCF5 | P49903 | Q9P2F9 |
| Q8IW00 | Q00169 | Q9UGK8 | Q9ULJ3 | O95140 | Q8NDX5-7 |
| O43257 | Q9BRL6 | Q8ND24 | Q9NVG8 | O00303 | Q96JB3 |
| Q5JTH9-2 | O14929 | Q5VX20 | Q16633 | Q9NWS0 | Q8IZ83 |
| Q13951-2 | O60291-2 | Q9NQ34 | Q5TB30 | Q9BRP8-2 | Q53GQ0 |
| Q8WV92 | E5RJF8 | Q8WUA4 | P11233 | Q15528 | Q96B97-2 |
| O77932 | A1A5D9 | Q6Q0C0 | X6RAL5 | Q04656-3 | Q8IXS8 |
| Q13112 | Q16851 | Q8WVM8 | Q9UBT6 | Q03933 | Q7Z3I7 |
| P63261 | Q8WUM4-2 | Q8WUM4 | O75940 | Q9UHN1 | Q03426 |
| Q86SX3 | Q9BT23 | Q9Y3E2 | O95747 | P55212 | Q9P2W9 |
| Q99504 | Q5TGY3 | Q6ZNA4-3 | Q15155 | Q8IZD4 | P62273 |
| O75592 | Q9Y3L3 | Q13105-2 | Q8N4N3 | Q6P4E1-4 | Q5W111 |
| P67812 | Q96LJ7 | Q9NRD9 | Q9BT17 | Q16513 | Q8IYX3-2 |
| R4GMX3 | Q5EE01 | Q7RTV0 | Q9NZD2 | P62314 | Q53FT3 |
| Q6PII3 | O43776 | Q9UL63 | O43660 | Q92562 | E7EVC7 |
| Q9UKG1 | Q9P2K2 | O60449-2 | Q9BSE4 | Q2M218 | O75607 |
| Q92624 | Q5NDL2 | Q86TI0 | Q9Y224 | Q15005 | P17097-2 |
| Q96GX9-3 | C9JXA7 | O00443 | O75351 | Q96PY6-3 | O95239 |
| P09417 | I6L8A6 | J3QR07 | Q5SY16 | Q6PI98-4 | O43516-3 |
| Q96AY2-2 | Q05639 | O43353 | Q5T4S7-2 | Q12906 | O75152 |
| Q9NR56-5 | Q9Y6W3 | P13667 | Q8N6M0 | Q9NR12-2 | Q8IZ40 |
| P32019 | P31949 | Q8N2W9 | Q9BYP7 | O43772 | Q9NPL8 |
| O95758-1 | P63313 | Q9C0F3 | Q96ES7 | Q9UIJ7 | Q9UJU6-3 |
| Q9BRZ2 | Q01970 | Q9Y4P1 | Q01650 | O00767 | P29692-3 |
| Q9Y2T2 | Q9H3R0 | Q9UJW0 | P22694-2 | Q9NSD4 | P0CG38 |
| Q96J01 | Q9P2Y5 | Q70Z35 | Q96MX3 | Q9BRS2 | Q9Y5W8 |
| Q96AT1 | Q9UHD2 | G3XAE9 | Q9NSU2 | P25490 | Q86W74 |
| Q9NP87 | Q8WVD5 | P05423 | Q9UHD1 | P30520 | C9J6P4 |
| O00429-2 | Q9Y2G5 | Q75QN2 | O15304 | Q9UBU8-2 | Q9H1P3 |
| Q14526 | A2RRP1 | Q9H2F5 | F8VYC4 | Q8N4W9 | Q6PK81 |
| Q96EV2 | O60678 | O75449 | Q92734 | Q9NUQ9 | Q9UPT5-6 |
| P35573 | H3BST1 | Q96MX6 | P60660-2 | J3KR97 | Q96K17 |
| Q9UJX5-3 | P05388 | Q53HV7 | O00268 | P51178-2 | Q9P013 |
| Q6DT37 | O75083 | P48059-5 | O43396 | Q9Y462 | Q13057-2 |
| Q9P0L0-2 | Q14118 | Q6P2C8-2 | G3V1V0 | Q9HCG1 | Q96GX9 |
| P40938 | P15170-3 | Q6ZTN6-3 | P49720 | Q8IUC8-3 | Q8WV22 |
| Q9HB96 | O43151 | Q96L34 | O75746 | Q9BSJ8 | O43933 |
| O96008 | Q6P3X3 | O60826 | P54725 | Q15018 | Q9BYX7 |
| Q9NWH9 | Q9NUP1 | Q16643-3 | Q9UHB7 | P51114-2 | Q7RTV3 |
| Q8N5M4 | O15260 | P0C2W1 | P30419 | O60884 | P78312 |
| P18583-5 | P43487-2 | P43487 | Q15014 | Q96IZ6 | Q13885 |
| P19634 | O43583 | P50990 | P18440 | P62745 | Q14240-2 |
| Q14240 | Q9NZ09-4 | Q92560 | P53384 | Q7Z2W4 | Q15034 |
| Q13423 | Q13042 | O15067 | Q9Y2G8 | Q7Z4V5 | Q9BXR6 |
| O75400 | Q6ZWJ1 | Q9H8Y5 | P62913 | P36508 | Q8NDZ2 |
| Q9BRG1 | Q56VL3 | P15880 | Q8IY57-5 | Q9C0B5 | O00299 |
| E2QRJ6 | O60739 | Q8TF32 | Q9NVT9 | Q7Z5G4 | Q9BVM2 |
| Q9H204 | Q01081 | O15037 | Q8N2C7 | A6NDU8 | O14709 |
| Q2TAA5 | B7ZL88 | Q01518 | P35790 | O43390-2 | Q8WYP5-2 |
| Q96QU8 | E9PNI7 | Q6IQ19 | P23443 | Q14004-2 | Q9UKK6 |
| A6NNY8 | O00716 | O95153-3 | Q96PV7 | Q8NBL1 | O60563 |
| Q96K49 | P12268 | O60711 | P58557 | Q9UMZ2 | Q9NUD7 |
| P62308 | Q5D1E8 | Q969Q6 | O60573 | Q5T3J3 | Q9H3F6 |
| G3V1Q4 | O60287 | O75427 | Q9H553 | Q96KG9 | Q9H0F6 |
| Q66K79 | Q92747 | Q86VN1 | Q8IW41 | Q96ME1-4 | P25440 |
| Q3LXA3 | A9UHW6 | Q9P0L0 | Q96T23 | Q5FWF6 | Q9GZN8-2 |
| Q8IWY9 | O15050 | Q13547 | Q14160-3 | P28676 | Q6BDS2 |
| Q7Z2K6 | E7EX59 | Q5VYS8 | Q8WV44 | Q14686 | Q9H307 |
| P62701 | Q8TAU3 | Q9NTJ5 | Q02388 | Q7Z6E9 | P47756-2 |
| Q9BZL1 | Q0P5N6 | Q9C0B9 | Q8IXZ2 | Q02790 | O15523 |
| Q9H4Z3 | Q6ZN18 | Q6IQ49 | Q8TAQ5 | Q9BT22 | Q86W50 |
| Q9UK61 | Q9H0A0 | O14818 | Q8NB90 | Q86WI1 | Q93096 |
| Q9BW66-3 | Q86YD1 | P40337 | E9PSH4 | Q16594 | Q6ZTR5 |
| P05388-2 | P52824 | Q2VY69 | Q9BX82 | P40429 | E9PJL5 |
| Q9NTX7 | P51114 | Q92994-3 | Q8NFU5 | Q96KR1 | P30048 |
| P00367 | Q96DU9 | P49674 | Q92966 | Q9NVH6-6 | Q04917 |
| Q01432-4 | P41208 | H3BUX2 | P08621 | Q96A73 | O60684 |
| Q9NR12 | P51571 | Q9Y512 | Q96RN5 | Q08ER8 | O14561 |
| P62879 | Q6P597-3 | Q9BZF3-5 | P84550 | Q7Z3T8 | Q9UPG8 |
| Q92918-2 | O15287 | Q06787-9 | A6NJG2 | Q92989 | Q3B7T1 |
| Q4KMP7 | Q16851-2 | Q9H583 | Q13438-7 | Q5JPE7 | Q9ULC3 |
| P61968 | Q969Z0 | O95801 | Q6P1Q9-2 | P68104 | P40818 |
| Q03111 | A0AVI2 | Q9UBS8 | Q9BVN2 | P19388 | O43295 |
| P51572-2 | Q96JN8 | O95402 | P52594-4 | Q96EL2 | Q96SB4 |
| O14979-3 | Q9NZ43 | Q9UI12 | Q8IV63 | P47755 | Q6PI26 |
| Q9BW91 | P61163 | G5EA36 | Q99583 | Q6ZMS7-2 | Q9UJ41 |
| O43660-2 | O14545 | A0A075B7G6 | Q02386 | Q5JVG2-3 | Q8N9K5 |
| O95278-3 | Q9UL15 | A6NED2 | Q86YS6 | O43852-3 | Q13144 |
| Q96QC0 | Q8TD19 | Q9C0B7 | Q9Y6X5 | P17676 | Q9UMX0 |
| O15014 | Q8N9R8-2 | P09012 | Q6ZS30 | Q969N2 | P42025 |
| Q8IYJ3 | Q86UT8 | P35249 | P54619-3 | P50991 | Q8TBE0 |
| Q5TEU4 | O95487-3 | Q5THJ4 | P16383 | Q96SQ5 | Q32NC0 |
| Q14320 | Q6P582 | Q9Y4Z0 | H7BXH9 | Q8TDD1 | J3KQA9 |
| Q9NVR2 | Q9BW62 | Q9H0W8 | Q01518-2 | Q9NXE8 | B8ZZN6 |
| Q07020 | Q6S8J3 | O00409 | P21399 | Q14789-2 | Q14789 |
| Q9UK96 | P08621-2 | P52907 | Q9BTA9-5 | Q13573 | Q92783 |
| Q5VIY5 | Q13627-2 | Q9NP80 | A8MUV8 | Q9H3R5 | Q9P1Y5-2 |
| P17844 | Q9UIW2 | Q9NRG9 | Q9NZ63 | Q9UBP0-2 | Q5RL73-2 |
| A2A3K4-2 | P08865 | Q9BRR6 | P61970 | P0C1Z6 | Q8NBQ5 |
| Q07866-8 | A8MUM1 | Q14694 | Q15637-6 | Q9BXR0 | Q5VW32 |
| Q86UQ0-3 | Q9H8M2-4 | Q9H1D9 | Q9NVV4 | P83436 | P50613 |
| Q8IZC7 | O60341 | Q53GS7 | Q9Y4H4 | P78417 | Q9P2G9 |
| Q9UBF6 | Q9UQ35 | Q9H2M9 | Q8IU60 | Q68DK2 | Q92828 |
| Q8TB40 | Q8N5F7 | Q9UP83-2 | P62979 | O15553 | Q9Y6V7 |
| Q9Y2H6 | P09234 | O75832 | Q96JC9 | Q9BY44 | Q6FIF0 |
| Q86T03-2 | Q6R327-3 | O43345 | Q6P5R6 | P12081 | Q9H5N1 |
| Q96B97 | Q9NQG5 | Q8IWE5 | J3QTA6 | Q69YQ0 | Q86XN6-2 |
| Q86XN6 | Q495B1 | Q12840 | O00329 | Q13356 | Q96SD1 |
| Q9UQR1 | A6NI79 | E9PK91 | P20336 | Q9ULE3 | P49773 |
| Q8IYS2-2 | Q8IWY8 | Q9HCM3-2 | Q9Y5S2 | P53367 | O15146 |
| P23610 | P50570 | P53814-6 | Q86UD4 | Q9NY93 | Q99547 |
| Q9UBV8 | Q9NRM2 | Q8IX15-3 | Q99576-3 | Q15796-2 | Q7Z7C8-2 |
| O94874 | Q9H4B0 | Q99615 | Q8WU76 | Q9C0D3 | F8VX10 |
| Q04721 | Q9P0T4 | Q8NFH3 | Q9UK45 | P17017 | Q9H7Z6-2 |
| A0A075B7E4 | Q13416 | Q8N9N7 | P17612 | Q93074-2 | Q9Y6J9 |
| Q13310-3 | Q13451 | Q8IWZ3-6 | Q8NB91 | A5A3E0 | J3KQK0 |
| Q9BQ67 | Q96GX2 | Q14566 | O95396 | Q8TCG2 | Q96LX8 |
| O60706-2 | Q7Z6B0 | Q92993-3 | P55884-2 | P68036 | Q9NYF8 |
| Q9H4M9 | Q6IA86-6 | Q12974 | O43633 | Q14CM0 | O60293 |
| P48637 | P46459 | O15533-3 | Q9BSB4 | P42768 | Q9BUA3 |
| Q9NY74 | G3V5N8 | Q96H20 | Q86UE4 | Q05996 | Q96K58-2 |
| O43432-3 | Q9NPB8 | Q9Y2M5 | P05160 | Q8WZA0-2 | P51149 |
| Q8WUA2 | Q9Y3C6 | Q9H6E4 | P00747 | Q15057 | Q9BYV8 |
| Q86U38 | Q14739 | Q9NYR9 | Q9HCN4 | Q07866-4 | O43149 |
| O95999 | Q8NDX6 | P54709 | Q02556 | Q7L5N1 | Q16560 |
| Q7Z3B3 | Q16881-6 | Q9NQX6 | Q9NR56 | F2Z2X4 | Q08722 |
| P38159 | P40306 | Q9H832 | C9JSP0 | Q8TDD1-2 | O94927 |
| P98170 | Q9GZQ3 | Q96HR8 | Q14590 | Q14139-2 | Q14457 |
| Q4L235 | Q9Y2W1 | P84090 | A8MXQ7 | Q92574 | A0A075B6E7 |
| Q9Y3T6 | O75179 | Q96QT6 | J3QS41 | Q96T60 | Q13895 |
| M0R2T5 | O43663 | P55160 | P05114 | E7EX17 | Q14159 |
| Q8N7H5 | Q04864-2 | Q14738 | O15379-2 | Q96KF7 | Q15717 |
| Q71RC2-4 | A6NN14 | Q9HAV0 | O14656 | Q9UBL3 | Q9HCI6 |
| Q96JG6 | P27482 | O95249 | Q16881-5 | Q96A33 | P0CJ78 |
| P62820 | O95149 | Q9NR28 | F8VWT9 | Q9BTA9 | Q9C005 |
| Q14289 | Q96CS2 | Q9Y547 | Q7L3B6 | O15131 | Q9H981 |
| Q8N2K1-3 | Q15027 | Q9NUS5 | Q4ADV7 | B4E0Y9 | Q14790-9 |
| Q9NWU2 | P23497 | Q15669 | Q96JD6 | Q5JRA6 | Q9BY42 |
| P68133 | Q9Y6W5 | O15392 | Q9UII5 | P13056 | Q16881 |
| Q8WUR7 | Q8TC05 | P49356 | Q96QR8 | Q6PJ61 | P17535 |
| O75113 | Q07866-9 | O15143 | O14578-4 | Q8N4U5 | Q8NBI5-2 |
| P00374 | Q00577 | P41250 | Q9Y2I8-2 | G3V325 | Q9NRY5 |
| Q9P2D0 | Q9GZT8 | P40692 | Q9NPB0 | Q8N163 | Q96F63 |
| Q86YW9 | Q9BYI3 | Q6H8Q1-9 | F8WBW8 | Q9Y2L1 | Q09328 |
| P62277 | P14649 | Q969K3-2 | Q9BZZ5-2 | Q92576 | P07437 |
| P53367-2 | Q8N8K9-3 | P30153 | Q9UBB6-2 | Q14004 | P61244 |
| Q86UY8 | Q8TEU7 | O95639 | Q0IIM8 | E7ETY2 | Q8WU90 |
| P04066 | P52597 | Q9BPW8 | P21283 | Q96EY4 | P11021 |
| Q96F24 | Q6YP21 | O60506 | O75323 | Q15738 | Q58FG0 |
| Q9NWW7 | Q5T160 | Q96NY9 | P30740 | Q9BQS8-3 | Q99459 |
| O14772 | Q9H7P9 | Q9NP77 | O14617-5 | P60660 | Q8NI22 |
| Q14699 | P33527 | H0YG20 | Q9Y6I9 | Q8IWZ8 | Q9UID3 |
| Q8NCC3 | Q9UKJ3 | Q15650 | Q8IY47 | O43567 | Q9UEY8 |
| O95456 | A6NMH8 | Q9P2P1 | P25789 | Q9C0F0 | Q86UX7 |
| Q9BTX1 | O75150 | O94986 | Q8NBL3 | P01871 | Q6NS38 |
| Q9Y6Q9 | Q9NW08 | Q9UJW7 | Q96HD1 | F8WCP5 | Q6NUQ1 |
| Q8IZQ5 | O43760-2 | Q8IYN0 | Q8IYT8 | P29597 | Q2NKX9 |
| Q99759-2 | I3L1I5 | C9JUH3 | Q9H9J4 | Q96JI7 | P50402 |
| P50502 | Q15459 | Q96F46 | P35251 | Q9NRG0 | E9PG19 |
| P61218 | Q9H0U9 | P60468 | O94864 | Q9UBP0 | Q92598 |
| Q9P2N5 | Q96E22 | P07686 | Q12893 | G3V4A5 | Q8IZ13 |
| P22626 | Q5TZA2 | Q9P289-3 | O60244 | Q96T52 | P01106-2 |
| Q8N5A5-2 | Q14055 | P0C0S5 | O14733-3 | Q9H974-4 | Q9H857-2 |
| Q6ZN28 | P05161 | Q9BZF9 | Q9HD40 | Q8N1G1 | Q16595 |
| Q8NFY9 | O60645-3 | Q9H5L6 | P49366 | Q6PI47-3 | Q8NEZ4-3 |
| O95988 | P62424 | O15226-2 | P53805 | O00429 | Q9HC77 |
| Q8NHQ9 | P61960 | Q96LA6 | F8WCN2 | Q6P9G9 | Q0VDD8-4 |
| Q9NQT4 | Q9BQ61 | Q8WXE1 | P30825 | Q9Y3V2 | O60256 |
| Q9Y2F5 | Q9Y5R8 | Q9H2P9-5 | P51946 | Q6P6C2 | Q9BPY3 |
| O95905-3 | Q9UMY4 | Q9H496 | Q7Z736 | Q9NZW5 | Q8N8R5 |
| Q5T8P6 | Q99816 | A0PJW6 | Q86UB9 | A8MU27 | O75376 |
| Q14CX7 | Q9BQ04 | H3BPX2 | Q00536-2 | Q8WUU5 | C9J4J4 |
| Q96G74-3 | Q5VZU9 | Q08209 | Q9H0U4 | Q15836 | O14979 |
| Q01664 | Q8TBA6 | Q13492 | O00411 | Q86WZ6 | O00221 |
| Q9BQI6 | Q6Y7W6 | Q9NRS6 | P61956 | P48059 | O75489 |
| Q96Q15 | Q71RC2-3 | Q8IWF6 | Q9BQS8-4 | Q2TAA2 | Q9P1U1 |
| Q9NVR0 | Q8NCN5 | P29144 | P49257 | Q96S82 | O95834-3 |
| Q6PCB5 | O14744 | Q86Y91-4 | Q9UFC0 | P62888 | O75400-3 |
| E9PRG8 | P31939 | Q5T200 | Q7Z4Q2 | Q9NUA8 | O15144 |
| P61289-2 | P50897 | Q13283 | O75665 | O00487 | Q86UE8-2 |
| Q8NEZ5 | Q6V1X1 | Q96I25 | M0QY76 | O00479 | O43677 |
| Q13257 | Q00978 | Q9P209 | P23193 | Q9BZ67 | Q9NVK5 |
| Q9H0A8 | Q9NX24 | O00178 | Q9NZT2 | Q9UJC3 | P49459 |
| O75586-2 | P30291 | Q9NUK0 | Q8TDM0-3 | Q5T8D3-3 | Q6ZVM7 |
| Q9Y294 | Q5VTR2 | Q9BZF2 | Q03518 | Q9NZN8 | Q6XE24-2 |
| Q9UK39 | Q16576 | Q8TDX9 | Q96GS6-2 | Q96T21 | Q86YA3 |
| Q68CZ6 | Q6DKI1 | Q16644 | O60885 | Q14511-3 | E7EVH7 |
| Q02543 | P25786-2 | Q8TBF4 | Q9NWF9-1 | Q92759 | P13637-2 |
| Q6L8Q7 | P31948 | Q86Y39 | P52741 | P31948-3 | Q9UNH7 |
| Q86Y56 | Q9H3W5 | P14373 | Q86W34 | P13747 | G3V1C3 |
| Q96HC4 | P45880 | Q9UKM9 | Q99567 | H0Y3P2 | Q9NVU7 |
| O15254 | Q96BZ8 | Q9UJS0 | P19387 | P17098 | P33076 |
| P50570-4 | K7ES30 | Q08752 | O00522 | E9PR30 | A0A075B752 |
| Q9NSG2 | Q13247 | Q9BV19 | B7Z6K7 | Q9H490 | Q9NUQ3 |
| Q8TCF1 | Q13554-5 | Q8N1F8 | Q15424 | Q8N129 | Q2PPJ7 |
| O43149-3 | Q99250 | Q9Y5Q8 | Q6V9R5 | Q7Z698 | P55072 |
| O00399 | Q9NRY4 | Q9Y519 | Q8TBX8 | Q9HA38 | P27986 |
| Q8WU39 | P53677 | Q9P107 | Q6NYC8 | Q96PK6-5 | P11441 |
| Q14677 | Q9C0C2 | P30613 | Q06323 | Q9BVC4 | O75886 |
| Q96N21 | Q14103-3 | Q9GZZ9 | Q9H9A5 | P16298-4 | A2RUC4 |
| Q15424-4 | Q13200 | Q9NV66 | Q9UGJ1 | P22681 | O94972 |
| Q7L2J0 | Q15059 | O15265-2 | Q8NI36 | Q86SX6 | Q9BZE9-2 |
| Q86TN4 | Q9NZD8 | Q5T1Z4 | P62241 | Q9BUV8-5 | Q6PJG2 |
| Q9UHR5 | O95670 | P31629 | Q96K80 | O95486 | Q15437 |
| P62304 | P62191 | Q07002-3 | Q6NSZ9 | P46781 | Q9UBU7 |
| Q9HA65 | Q9H3P7 | Q15544 | P55081 | Q9BRQ3 | Q96AZ6 |
| Q14671 | Q13330-3 | P42566 | Q8N668 | Q15048 | P38935 |
| Q9BQG0-2 | P11940 | Q9NQR4 | Q9UBB6 | Q9NUW8 | Q9Y4K4 |
| Q9NU23 | Q7Z5U6 | Q6ZMW2 | O15055 | J3KPD3 | Q13428-4 |
| Q8TAT6-2 | Q9BRP4 | Q9UIV1 | P14678-3 | O94760 | Q8N5L8 |
| A8MYG5 | P29083 | E7EW50 | P61221 | Q9Y5Y2 | Q8ND56-2 |
| Q2M296-3 | O60673 | P25208 | Q9BV44 | D6RG30 | Q15051 |
| Q02548 | A8MQT2-2 | P17858 | Q9NYK1 | P54198 | Q10589 |
| Q9Y673 | Q96DI7 | Q68DH5 | Q9H999 | P13686 | Q5T8P6-3 |
| P16066 | P29084 | P56192 | P17028 | Q13472 | Q13227 |
| O15063 | Q00839-2 | Q15165-1 | Q92610 | Q7Z2K8 | P07900-2 |
| O75874 | Q9BV38 | P35250 | Q6NYC1-3 | Q5JSP0 | Q15370 |
| Q9Y5Y0 | O94966-6 | Q9H4L5-3 | Q5PSV4 | B5MDF5 | O14802 |
| Q9Y277 | Q9NX46 | Q96P31-3 | O43524 | Q9BTY7 | P26599 |
| P62318 | Q9Y6J0 | O15400-2 | Q12899 | P48723 | P98175-2 |
| Q92917 | Q6SPF0 | Q96E39 | Q9Y2V7 | P23246 | P02786 |
| Q9BW71 | Q86WV7 | P56270 | Q9UHQ4 | Q14689 | O00148 |
| Q5BKZ1 | O15230 | Q92979 | Q8N5I4 | O43852-4 | Q86UP2 |
| Q16778 | F8W130 | D6RFG8 | B1AJZ9-3 | Q9BWK5 | O43242 |
| Q9H0R4 | Q8WU58 | O43741 | Q2YD98 | Q9UGU5 | P52272 |
| Q9NQG6 | Q9H2C0 | Q504Q3 | P51795-2 | P10398 | Q8WUH1 |
| Q9UPZ3 | Q6NVY1 | Q14676 | Q8N5M9 | O75494 | Q13190 |
| Q5VVJ2 | Q9NYV6 | O14556 | Q8TDW7 | O94913 | Q12933-2 |
| Q9C0C7 | Q8TCU4 | P40616 | P35658-3 | Q9H8G1 | O75636 |
| O00505 | Q96DX4 | Q6NUN9-2 | Q07890 | O94921 | P22059 |
| Q9UIF8 | P10109 | Q8N4P6 | P54920 | O14965 | O15438-4 |
| Q96QK1 | O15381 | Q96DN5 | O95793-3 | Q66GS9 | Q9H6R7 |
| Q16560-2 | Q96EX1 | O75794 | Q9H488 | Q5SZL2-4 | Q8IY37 |
| O43805 | Q5VTL8 | E9PEJ6 | Q96MT3 | P05455 | Q5TC84 |
| P08048 | P13010 | Q92797 | Q96BN8 | P62328 | Q8NEY1 |
| P25686 | Q8WX92 | Q9H7Z3 | Q9NU19 | Q8WWY3 | P46782 |
| Q9UHV7 | Q6NXT4-2 | Q4G0F5 | Q9UFG5 | P49761-1 | Q96EV8 |
| P35244 | Q9H0S4 | Q6PJI9 | Q9H9Q4 | Q96JQ0 | Q9UKY7 |
| Q58FF6 | P42226 | P35658 | O15015-2 | Q86TI2 | Q9UHI6 |
| Q14680 | P55795 | Q9BW72 | Q96RS0 | O95391 | P00519-2 |
| Q15007 | Q659C4 | Q96HY6 | Q96PY5-3 | Q5MNZ9 | P84095 |
| Q9Y6N5 | Q8TAD8 | Q9BUR5 | Q9UJJ7 | P14209 | Q9H0X4 |
| B9A054 | Q8WWM7 | P60983 | P31153 | Q9Y2L5 | Q15269 |
| Q8NC54 | Q8NBP0 | Q06787 | Q06787-7 | Q8N806 | Q9H444 |
| Q9NZN5 | O15514 | P36897-2 | Q9BTV4 | Q96QE3 | Q5TGZ0 |
| P52209 | Q5M9N0 | P53804 | E9PDN6 | Q9UHF7-2 | Q9BZE4 |
| Q96EP1 | Q9P2J5 | O43290 | Q13838 | Q6P4R8-2 | Q6NW29 |
| Q8NHM5 | Q8N5K1 | Q9Y253 | P42575 | O43148-2 | B4DZM6 |
| Q01804 | Q8IYD1 | Q6UX04 | O00571 | B4DWJ3 | Q96P11-2 |
| Q9Y2U5 | Q14331 | P41229 | Q8IYL3 | O75529 | Q9NXR1-2 |
| P0C881 | Q9BXJ9 | Q6NXT1 | P40425 | Q04206 | Q08AM6 |
| J3KPZ4 | Q96GC9 | Q8NBJ5 | Q9NS40 | Q96LA6-3 | Q16698 |
| Q5J8M3 | Q9NTW7 | Q8NB66 | Q7Z392 | P61978-3 | Q9NU22 |
| Q12962 | Q9Y5U2 | P63208 | Q9H9Q2 | P62495 | Q9NQE9 |
| Q9Y546 | Q8IUR7 | Q9NVW2 | Q9ULA0 | Q9Y572 | Q12972 |
| P09382 | Q6GMV3 | Q9BWT7 | Q712K3 | Q4G0J3 | Q6DKJ4 |
| Q9BVL2 | Q9NVC6 | Q8N128-2 | Q99747 | P36969-2 | Q9Y5G7 |
| P63162-2 | P25685 | Q9Y282-3 | Q9Y6M5 | Q9BSQ5 | Q0VG06 |
| Q9BUQ8 | P51449 | Q8WZ60 | P98161 | P28070 | Q9H8T0-2 |
| P25788 | Q8N6T7 | Q8IXQ3 | P32969 | J3KNB0 | P42771 |
| Q15233 | Q16401 | P18124 | Q9UBM7 | Q6FI13 | O60814 |
| Q9Y252 | P26038 | P49368 | O75175 | Q9Y6M4-4 | P09525 |
| Q15369 | Q5VV52-3 | Q5SNT2 | Q9H477 | P54840 | A1L0T0 |
| Q9BWM5 | Q86WA8 | P33992 | P58876 | Q8IXS6-2 | P12956 |
| P07195 | Q9NUL3 | Q5EBL8-2 | P48426 | Q96GD0 | P17020 |
| Q71RG4 | O95825 | Q08431 | Q9Y2Q5 | Q92782-2 | Q9BYT9 |
| Q8TDR2 | Q969V6 | P61129 | J3KTL2 | P42345 | P34897 |
| Q9BVK6 | O43164 | P20916 | Q9U110-3 | Q969M3-3 | Q99707 |
| Q8WU79 | O95376 | Q9BZ95 | Q9P2K3-3 | J3KNP4 | P0C7V5 |
| Q9H8H3 | O15534 | Q8N680 | C9K0J5 | Q12996 | P23469-2 |
| Q15831 | Q9BSJ5 | Q8IUH3 | Q9H6T3 | Q8WUD4 | Q9UDT6 |
| Q6P1M0 | Q969R8 | P14866 | Q8N999 | Q05D60 | Q8NDX1 |
| Q969J2 | O95870 | Q86VV8 | Q8N2M8 | O14964 | Q9UQN3 |
| Q9BT09 | Q9Y5K8 | Q8N6T3 | Q99879 | P85037 | Q71SY5-4 |
| Q9H0K1 | P54105 | P52294 | Q08257 | Q8WUY9 | Q75N03 |
| P15822 | Q9UM13 | Q01105-2 | P55347 | J3KR51 | Q9Y535 |
| P07199 | Q14677-3 | Q9GZL7 | O00231 | Q7Z4G1 | Q969J3 |
| Q8NDT2 | Q5W0Z9-3 | G3V256 | Q9Y2P8 | Q04637-8 | Q96SZ4-2 |
| E7ERS3 | P20618 | Q8TE04 | O43414 | P40925-3 | P04632 |
| P56270-2 | Q03164-3 | O43791 | Q9NPC7 | P36954 | Q9P0J7 |
| Q14586 | Q5T2D3 | Q2WGJ9 | L0R819 | P04179 | P49959 |
| F8W7U8 | Q6P9B6 | Q96ST3 | M0R2B7 | Q6PJT7-2 | Q9UL03 |
| Q92831 | Q9NX31 | P00734 | Q9H8W4 | Q9HBE1 | Q5THK1 |
| Q5W0B1 | Q12830 | Q04637-4 | Q5VV42 | P11474 | Q5HYI7 |
| P68366 | Q96IZ0 | Q9C0D7-2 | P09913 | P17152 | Q9NYV4 |
| Q6ZS27-3 | Q93075 | Q8TEP8-3 | P45984 | P55786 | O43422 |
| P50583 | Q9BWF3 | P20290 | O94915 | Q00325-2 | Q70EL2 |
| Q92611 | O60271 | Q9BSR8 | Q9UKI8-5 | Q99700 | Q08170 |
| P04183 | P17036 | Q658Y4 | O43513 | E9PF55 | Q6DHV5-2 |
| Q14135 | Q92551 | Q9UNS2 | A6NML8 | Q9Y221 | Q03188 |
| B4DJL6 | J3KQ70 | Q5SQS7 | Q8N1G2 | Q9H3Z4 | P78344 |
| Q86U42 | P08575 | P51587 | Q9P086 | Q14344 | Q04637-5 |
| P61978-2 | Q9ULG1 | O00754 | O75569 | O75943 | O00193 |
| P20290-2 | Q9BTD8 | Q9BU76 | M0QYZ2 | Q14318-2 | O95926 |
| Q9NUZ1-4 | P10075 | Q9H930 | Q15329 | Q9HAS0 | Q68CP4 |
| Q9Y3C7 | Q14CZ0 | P51790 | P49736 | P63279 | R4GMW7 |
| Q6N069 | Q9UGV2 | Q9BUR4 | Q8WXG6-4 | Q8TF68 | Q04637 |
| P55771 | Q9UI47 | Q96HP0 | O43292 | Q9H4L5 | Q8WW59 |
| Q7L1V2 | Q8TBC3 | O75717 | Q86VH2 | P84022 | Q15006 |
| Q15020 | Q96LT9 | O75531 | Q9NV70 | Q14318 | P04080 |
| B0YIW6 | Q9H115 | Q9Y6D9 | Q9NRG4 | Q8N960 | Q9H6W3 |
| Q01658 | Q9UMS4 | Q8NC56 | P16885 | Q96EH5 | A3KN83-2 |
| Q7Z403 | Q5SRQ6 | Q15742 | Q7L3S4 | Q8TEB1 | Q9Y3P9 |
| H0YKT5 | Q9UH92 | Q9UPM8 | Q6P444 | Q92905 | Q6IQ20 |
| Q8NBZ0 | P57737-3 | Q2KHT3 | P46531 | Q9H254 | P48634 |
| Q6EKJ0 | F5H619 | H3BUF6 | Q14CS0 | O60583-2 | P62195 |
| H7BY22 | A7E2V4-2 | Q9Y5V0 | Q9HB09 | P13051 | Q9Y4D8-5 |
| Q86YV9 | P53803 | Q9UJT0 | Q86XR8 | O60294 | P62280 |
| Q00839 | P49792 | Q06187 | P50452 | Q96SW2 | Q5QPK2 |
| B1AKR6 | Q9BXW7 | Q14103-4 | Q14209 | Q8TC44 | Q9Y2R4 |
| Q9BY43-2 | Q9Y5Z9 | Q8IXQ4 | K7EMK7 | P17987 | Q99871-2 |
| Q96QT4 | Q96MT8 | Q92794 | P01889 | P21359 | Q9UBU9 |
| Q5BJF6 | P49711 | Q9BQE4 | Q5T8D3 | Q8IY92 | P49458 |
| Q9H0E3 | Q96B26 | Q6EEV4-2 | U3KQM8 | O95394-3 | Q8WTW3 |
| Q6KCM7 | Q8IVQ6 | O43815 | Q9ULH7-5 | P31751 | Q9BPZ7 |
| Q86VE0 | Q9BTL3 | Q96NW4 | P0CG12 | O95630 | H0Y860 |
| Q6WKZ4 | O94806 | P15311 | Q9GZM8-2 | F5H2M7 | Q6ZMI0 |
| Q7Z460-4 | O60524 | Q0VDF9 | Q9Y5W7 | E7EQ45 | O15111 |
| O14497 | O00750 | Q5BJH7 | Q9NVP2 | Q9UK59 | Q6NUQ4 |
| Q499Z4 | Q00765 | Q7Z569 | Q9H6U6-7 | Q9H8W3 | Q8WVX9 |
| Q9BRR8 | O43542 | Q13813-2 | O00629 | Q04760 | Q9UPT6 |
| Q13490 | Q92541 | O15091 | Q9BRR9-2 | O00401 | Q9UKF6 |
| Q5T1V6 | Q9UMR2 | Q9P2D3 | P54578 | Q567U6 | O14602 |
| P04040 | Q8NI08 | Q14690 | P22314 | P23528 | O14770 |
| P26572 | P51956 | Q8NDM7 | P15336-5 | O95260 | Q56UN5 |
| Q8NG68 | Q8WVL7 | Q02040 | Q13596 | P52948 | O60336 |
| P11836 | Q7Z460 | Q96S59 | Q9NYZ3 | O75340 | Q14181 |
| P27708 | P16104 | Q9BU23 | Q86W56 | P61201 | P42338 |
| Q76L83 | Q6ZUM4 | Q6ZUJ8-2 | Q15750 | A6H8Y1 | Q9Y333 |
| Q9H1K0 | Q15696 | Q9H2D6 | Q8TDC3-2 | Q96EX3 | Q9UHX1-2 |
| O75093 | Q9BZ95-3 | Q92890-1 | Q9NZQ3 | Q9UBT2 | P17029 |
| O75554 | P51608-2 | P35611-3 | Q5QP82-2 | Q13148 | Q96QD9 |
| Q96MW5 | Q01780 | Q9H3C7 | O15228 | J3KNF8 | Q9BS34 |
| Q8WVC0 | Q9P260 | Q96PK6 | Q96Q27-2 | S4R3H4 | Q9BVA0 |
| P60842 | Q9NPE3 | Q8N6S5 | Q6NXE6 | P46060 | E9PBK7 |
| P98164 | Q6YHU6 | Q9NZL4 | Q8NE35 | Q86Z02 | O15084-1 |
| Q9BVV6-3 | Q05469 | O95835 | Q96S66 | Q9NRL3-3 | Q9HB90 |
| P55039 | Q9BR77 | Q9Y5B0 | Q06265-2 | Q8NEM7-3 | Q9P2J9 |
| Q8WXR4-7 | Q15032-3 | Q92575 | P12110 | Q15572-6 | P78317 |
| Q9H5V7 | O00533-2 | P55198 | O43251-8 | Q9UFH2 | P06730-2 |
| Q96Q05-2 | Q9BT92 | Q9UI10 | P17544-6 | B4DY08 | Q15648 |
| O60271-4 | Q9H0L4 | C9JCK5 | Q9NZC9 | O95707 | Q9H6R4 |
| O94906 | P11831 | Q9BUB4 | Q6DKK2 | J3KPY6 | Q01813 |
| Q9H7X3 | Q2TAL8 | Q52LJ0-2 | P52298 | P68363 | Q96DX7 |
| O75934 | P04843 | Q99543 | P27695 | Q08334 | P00491 |
| O95248-4 | Q5T0B9 | P42331-4 | Q8IV38 | Q9NVM6 | Q969L4 |
| P20338 | G5E9V6 | Q5T2E6 | Q06413-5 | P55201-2 | Q8N139 |
| B5MDU6 | E7EQR8 | P22670 | Q13619 | O14976 | O43683 |
| Q9Y2V2 | Q9UIA9 | Q99460 | Q96N46 | Q14964 | Q9UKX7 |
| P23396 | Q9UH03 | Q96SN8 | Q04446 | O76003 | Q9Y6C9 |
| P38606 | Q15013-3 | O43502 | P61981 | Q92643 | P30307 |
| Q9UQL6-3 | Q9NUL3-7 | Q96CB8 | Q8WYH8 | Q9BY32 | Q05048 |
| P06454 | Q9BZ29-4 | P04035-3 | P23759 | Q9P0J1-2 | Q13813 |
| Q96DF8 | Q14568 | Q15029 | Q8WTT2 | Q9ULR0-1 | Q08999 |
| Q08623 | Q9NRG1 | Q7Z5W3 | Q9BRP7 | Q96JB2 | P62873 |
| Q9H3P2 | A6NIV6 | P04233 | Q562R1 | Q9NRF2 | H9KV53 |
| P29218 | Q9UJA3 | G5EA30 | Q02978 | O15127 | P09496-2 |
| Q9UK41-2 | Q6PCB8 | P62987 | B2RNG4 | Q9UJX3 | Q86YV0 |
| Q96S52 | Q96N64 | P32970 | Q6P2P2 | Q9Y2I1 | Q14145 |
| Q13595 | Q14161 | Q8N6H7 | Q15075 | Q9Y5A6 | P49754 |
| O75896 | P42126 | Q9H6Z4-2 | Q8WTS6 | P04220 | Q00403 |
| Q14151-2 | O96013 | Q9UK53-2 | P53779 | Q8WXI7 | Q15386 |
| J3KNZ9 | Q17RS7 | Q58FG1 | Q9H299 | O15318 | A6NKF1 |
| O43929 | Q13371 | Q92859 | P48736 | Q9HDC9 | P0CAP2 |
| Q969F9 | Q96HI0 | J3KN59 | Q9UHX1 | B4DWG0 | Q86SQ9-2 |
| Q12874 | P36894 | Q86U44 | Q86XK3-2 | O14763 | Q9H4L4 |
| Q9UGH3 | P61599 | Q96FC9-2 | Q58A45 | Q9NR09 | O75534-2 |
| Q13868 | Q96HR3 | Q9ULD9 | Q96PM5 | O75496 | A5PLN9-5 |
| Q9NW82 | Q9P2G3 | Q1MSJ5 | Q8N9Q2 | P12004 | Q96RU2-2 |
| Q9BSF4 | Q9BT78 | Q8TF74 | A6NFI3 | Q8NEB9 | Q13427 |
| Q86YV5 | Q7Z591-5 | Q8TAA5 | Q15813 | Q9UJA5 | P48739 |
| O95644 | O95644-2 | P63104 | O60701 | O95816 | Q9BT25 |
| Q9H0E3-3 | Q9Y696 | Q9Y312 | P46734 | P22087 | P52789 |
| Q13561-2 | Q9NVM9 | Q9NXS2 | O15018 | O60583 | Q9UBQ5 |
| Q9UKV5 | Q9Y3T9 | Q14164 | F2Z2W7 | P49753 | Q9HCE0 |
| P0CB38 | Q9HB58-7 | Q9H0D6 | O75691 | O95671 | Q96K75 |
| Q9H2J4 | E7EWQ4 | J3KR33 | Q9Y2G3 | Q8NFH4 | Q9NPJ8 |
| Q9Y2E4 | Q14C86-6 | Q9UJ78 | O95613 | O95219 | Q12772 |
| Q9UBI9 | Q13825 | O94817 | P53992 | J3QLM1 | Q9HD20 |
| Q9HD20-2 | Q15390 | Q9P253 | P13797 | Q6ZSZ5 | O14908 |
| O95159 | P13535 | E9PAU2 | Q9UDY8 | Q9NUU7 | Q9UKZ1 |
| O75164 | P36915 | Q8IV04 | F8W8D3 | Q07352 | Q8WVQ1 |
| Q68CZ2 | Q96RL1 | Q9P2R3-2 | B3KS98 | Q9NUP9 | Q14761 |
| P56181 | E7EX90 | Q9NS86 | Q9BRT6 | O60308 | Q9H6Z4 |
| Q9UM11 | P01116-2 | O60749 | A6NC98 | P52566 | Q9BUL5 |
| P28068 | B7WPE2 | Q16629 | P39748 | P50395 | Q9P2R6 |
| P13473-3 | Q92945 | O95166 | Q9H5V9 | Q8N684-3 | P07384 |
| Q01844 | O43264 | Q9BXS6-7 | J3KQJ1 | Q9Y2X3 | O75964 |
| Q9HCI7 | F5GY05 | Q96HU1-2 | Q5F1R6-3 | Q8IVH8 | O60237 |
| P45974-2 | Q9BV73 | Q86Y07 | Q6P5Z2 | O60337 | Q96EP0 |
| Q9Y5P4-3 | Q05519 | Q7Z591 | P31946 | P31946-2 | Q7L273 |
| Q9BWS9 | Q8NFZ0-2 | P49915 | H3BRJ5 | Q9Y4E5-4 | C9JEH3 |
| O75330-3 | Q15631 | Q9NS73 | Q8NEK5-2 | O15382 | Q96DR4 |
| P51648-2 | Q9BTE6 | P63146 | P41182 | Q9H0K6 | Q96PE3 |
| P08243 | O95433 | Q12788 | Q86X55 | P14550 | Q96AX1 |
| Q9HC38-2 | Q2QGD7 | O14686-3 | Q8N4S0 | P60900 | Q9Y217 |
| Q6ZSR9 | J3KMZ7 | Q9H1A3 | P29374 | Q9Y2W2 | Q587I9 |
| Q9NQW1 | Q16204 | Q8NFW8 | Q9Y4F3 | O75787 | P49427 |
| Q9UHX1-5 | E9PES4 | Q9H7L9 | P33240 | O00151 | Q15008 |
| Q9Y6I3 | Q9NW68 | Q12834 | Q8NEL9 | P20671 | P0C0S8 |
| Q93077 | Q13867 | P42331 | Q9HC10 | O75935 | P32004 |
| Q969V5 | X6RLX0 | Q8TB96 | Q15431 | Q9HCE7 | Q8TEQ6 |
| Q5QP82 | P23368 | Q969F1 | P27361 | Q96MF7 | P20929-2 |
| P26641 | O60232 | P01111 | Q9Y2U9 | K7ERU7 | Q9Y3F4 |
| O60925 | Q9H5H4 | Q9P2G1 | Q9NQ29 | Q9NV88 | Q86W92 |
| Q6P1M9 | Q3YEC7 | Q9BXS6 | Q6QNY0 | Q9UHL4 | Q9Y6X3 |
| Q6AI39 | Q8IWV8 | O60427 | P13489 | Q9BS16 | P07902 |
| Q9NSD9 | P27694 | Q8IY95 | O75208 | O95232 | Q9BVI4 |
| Q14847 | Q86TX2 | Q13748 | Q96AG4 | P04233-2 | O75534 |
| Q8NBF6 | G8JLB6 | M0QY62 | P35998 | O43237 | O75446 |
| Q92888-3 | Q8N9N2-2 | Q9BXS6-6 | P25787 | Q13610 | Q9H944 |
| Q9H116 | Q16526 | P27144 | P13994 | Q9UBE0 | Q5VZF2 |
| Q9NVU0 | Q96IK1 | Q9NS56 | Q5SQI0 | Q9NNW7 | Q96FW1 |
| Q86VE9-4 | Q9Y3D2 | Q96EP5 | J3KNI1 | Q92615 | P36543 |
| Q15973 | Q92804 | Q6QNY1 | Q3SXM0 | Q5T9S5 | O15525 |
| Q6P4A7 | P41223 | Q5RKV6 | Q6ZUJ8 | Q9NVF7 | Q6PJT7-3 |
| P01112 | Q9Y6G5 | Q9C0B2 | O00458 | O43324 | Q9Y320 |
| Q66K14 | Q16563-2 | Q6P3S6 | Q96DY2 | A2BF26 | P61769 |
| Q7Z7A1 | Q5VWJ9 | P17812 | P61313 | O95793 | Q56NI9 |
| Q5T8I9 | Q6B0I6 | Q92738 | P62875 | P52434 | A2AJT9 |
| P37108 | O95985 | Q9H0J9 | Q15067-2 | Q00537-2 | Q9UKV8 |
| Q9UBI4 | Q96BZ9-3 | Q8WUX1 | Q14563 | C9JCC6 | Q9BUW7 |
| Q9H4H8 | P50548 | Q9C0D2-3 | Q5T6J7-2 | P17031 | Q9UKV3-3 |
| Q15003 | Q9UJV9 | Q15102 | O75151 | Q9NSI6-2 | Q9GZS3 |
| Q9Y3B2 | Q12965 | Q9H1U9 | Q03001 | Q86YT6 | Q12851 |
| P45954 | Q16342 | Q6P2E9 | Q6NXR4 | E7EWN3 | Q13751 |
| Q8N653 | Q8N5C7 | Q15493 | Q8NFU7 | Q7Z340 | Q7Z624 |
| O60513 | Q8WUJ0 | Q9NZB2-6 | Q8WUZ0-2 | P13796 | Q16637 |
| Q86YC2 | Q9Y3S1 | Q15019-2 | J3KQ48 | Q99487 | Q9NW38-2 |
| Q5W0U4 | O60869 | Q16514 | Q9BQE3 | Q96CS4 | Q9UET6 |
| Q86U06 | O75306 | P31146 | Q8NEY8-3 | Q6PJG6 | Q8TEA1 |
| Q9NUG6 | Q9UHG3 | Q86UA6-8 | P02788 | G5E9N2 | P00505 |
| P00441 | Q9UL59 | Q96JF6 | Q52M93 | P99999 | Q9HAN9 |
| P01024 | P01023 | P20742 | P19823 | P17900 | P51508 |
| P07996 | P02774-3 | Q16854 | P23142 | P0C0L5 | P98182 |
| Q15063 | Q13424 | Q9BSG1 | Q9UIE0 | O95363 | Q6PF04 |
| Q06033 | P02751-15 | P04733 | Q5TEC3 | Q9BSK2 | P08697 |
| P47989 | Q86VK4-5 | B7ZKJ8 | Q9BU19 | Q92902 | Q8IVS8 |
| O15239 | Q86SE9 | O75298 | Q8NE62 | P54284 | Q9HDC5 |
| P51884 | A8MX12 | P36955 | Q9Y6T7 | Q9UPV9 | Q8WUY8 |
| Q6ICH7 | Q5VYJ5 | P50454 | Q9NWT8 | O94941 | Q16772 |
| P0CG23 | Q15406 | Q6ZNH5 | Q8TCU6 | Q12766 | Q8N7B6 |
| Q9H0V9-2 | Q9NQV7 | J3KTH2 | P01579 | Q13609 | P08603 |
| P01008 | Q9H6S1 | P43652 | Q96T25 | O95445 | Q5JUQ0 |
| Q6ZSB9 | E9PIM0 | Q494V2-2 | Q86UK5 | Q12923-4 | Q13563 |
| P08151 | | | | | |

### Use of Signature Genes

The invention provides BCL-2 related gene signatures for use in a variety of diagnostic and/or therapeutic indications. For example, the invention provides BCL-2 related signatures that are useful in a variety of diagnostic and/or therapeutic indications. In certain embodiments, the invention provides for signatures of BCL-2 inhibitor resistance.

“Signatures” in the context of the present invention encompasses, without limitation nucleic acids, together with their polymorphisms, mutations, variants, modifications, subunits, fragments, and other analytes or sample-derived measures.

Exemplary signatures are shown in Tables 1, 2, 3 and 4 and are collectively referred to herein as, inter alia, “BCL-2 associated genes,” “BCL-2 inhibitor resistance associated genes,” “BCL-2-associated nucleic acids,” “signature genes,” or “signature nucleic acids.”

These signatures are useful in methods of diagnosing, prognosing and/or staging a treatment or response in a subject by detecting a first level of expression, activity and/or function of one or more signature genes or one or more products of one or more signature genes selected from those listed in Tables 1, 2, 3 and 4 and comparing the detected level to a control of level of signature gene or gene product expression, activity and/or function, wherein a difference in the detected level and the control level indicates that the presence of a response in the subject.

These signatures are useful in methods of monitoring an treatment or response in a subject by detecting a level of expression, activity and/or function of one or more signature genes or one or more products of one or more signature genes selected from those listed in Tables 1, 2, 3 and 4 at a first time point, detecting a level of expression, activity and/or function of one or more signature genes or one or more products of one or more signature genes selected from those listed in Tables 1, 2, 3 and 4 at a second time point, and comparing the first detected level of expression, activity and/or function with the second detected level of expression, activity and/or function, wherein a change in the first and second detected levels indicates an effect of the treatment of change in the response in the subject.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). The term “monitoring” generally refers to the follow-up of a disease or a condition in a subject for any changes which may occur over time.

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

These signatures are useful in methods of identifying patient populations at risk or suffering from a BCL-2 or BCL-2 family driven disease or disorder based on a detected level of expression, activity and/or function of one or more signature genes or one or more products of one or more signature genes selected from those listed in Tables 1, 2, 3 and 4. These signatures are also useful in monitoring subjects undergoing treatments and therapies to determine efficaciousness of the treatment or therapy. These signatures are also useful in monitoring subjects undergoing treatments and therapies for aberrant BCL-2 or BCL-2 family driven disease(s) or disorder(s) to determine whether the patient is responsive to the treatment or therapy. These signatures are also useful for selecting or modifying therapies and treatments that would be efficacious in treating, delaying the progression of or otherwise ameliorating a symptom of a BCL-2 or BCL-2 family driven disease or disorder. The signatures provided herein are useful for selecting a group of patients at a specific state of a disease with accuracy that facilitates selection of treatments.

In certain embodiments, the signature genes are used to determine BCL-2 responsive pathways. For example, groups of signature genes may indicate pathways that are differentially active or inactive in BCL-2 inhibitor resistant subjects. In contrast to gene-level analysis, pathway-level geneset enrichment analysis (GSEA) based on RNA expression or protein expression can be used to reveal significantly enriched pathways. The analysis of data for the BCL-2 responsive genes revealed 35 significantly enriched pathways (Table 5). Consistent with pathway-level results from Applicants' gain- and loss-of-function screens (Tables 1 and 2), positively regulated pathways included lymphoid differentiation and chromatin maintenance, while top negatively regulated pathways related to metabolism and the endoplasmic reticulum. In addition, as observed in Applicants' functional genomics screens, the most coordinately upregulated transcripts and proteins (Tables 3 and 4) originated from genes critical to cellular metabolism (AOX1, GLUL, PAPSS1, GATM, TSTD1, GALM, FBP1). The other upregulated transcripts/proteins highlighted other mechanisms of interest, including cell cycle regulation (CDK6, CDKN1A [encoding p21], TT39C), B-cell biology (DOCK10) as well as autophagy (DENND3, OPTN) and reactive oxygen species generation (CYBB). In certain embodiments, pathway specific biomarkers may be used in methods of diagnosing, prognosing and/or staging a treatment or response in a subject. For example, detecting metabolites or intermediates related to OXPHOS or glycolysis in a subject tumor sample can be used in monitoring, diagnosing, prognosing and/or staging a treatment or response. The pathways may indicate appropriate treatments that modulate such pathways. Screening for agents capable of modulating pathways are described further herein.

TABLE 5
GENESET ENRICHMENT ANALYSIS

| | | | | |
|---|---|---|---|---|
| NAME | SIZE | NES | NOM p-val | FDR q-val |

POSITIVELY REGULATED PATHWAYS

| | | | | |
|---|---|---|---|---|
| PCG PROTEIN COMPLEX | 43 | 1.9235198 | 0.0 | 0.014543598 |
| STEROID HYDROXYLASE ACTIVITY | 24 | 1.8683462 | 0.0 | 0.05157862 |
| T CELL DIFFERENTIATION | 117 | 1.8198463 | 0.0 | 0.120191924 |
| LYMPHOCYTE DIFFERENTIATION | 193 | 1.8131264 | 0.0 | 0.10498436 |
| POSITIVE REGULATION OF STRIATED | 45 | 1.804444 | 0.0 | 0.1016172 |
| MUSCLE CELL DIFFERENTIATION | | | | |
| DNA PACKAGING COMPLEX | 100 | 1.7810507 | 0.0 | 0.14139923 |
| REGULATION OF SYNAPTIC TRANSMISSION GABAERGIC | 24 | 1.7761554 | 0.001980198 | 0.13557017 |
| VENTRAL SPINAL CORD DEVELOPMENT | 32 | 1.7603424 | 0.0 | 0.16121376 |
| SYMPATHETIC NERVOUS SYSTEM DEVELOPMENT | 17 | 1.7601466 | 0.0 | 0.14384659 |
| BILE ACID TRANSMEMBRANE TRANSPORTER ACTIVITY | 15 | 1.7552881 | 0.0 | 0.14266519 |
| POSITIVE_REGULATION_OF_HEART_RATE | 19 | 1.751453 | 0.008032128 | 0.14007458 |
| PALATE_DEVELOPMENT | 75 | 1.7337528 | 0.0 | 0.18172325 |
| POSITIVE REGULATION OF SYNAPTIC | 16 | 1.7321408 | 0.0 | 0.17230986 |
| TRANSMISSION GLUTAMATERGIC | | | | |
| POSITIVE_REGULATION_OF_DNA_REPLICATION | 79 | 1.7309617 | 0.0 | 0.16379696 |
| RETINAL_GANGLION_CELL_AXON_GUIDANCE | 15 | 1.7172953 | 0.0018552876 | 0.19632848 |
| CHROMATIN_ASSEMBLY_OR_DISASSEMBLY | 160 | 1.7099936 | 0.0 | 0.20630613 |
| SOMATIC_STEM_CELL_POPULATION_MAINTENANCE | 58 | 1.709694 | 0.0 | 0.19516784 |
| RESPONSE_TO_THYROID_HORMONE | 20 | 1.7076536 | 0.0 | 0.18985793 |
| DORSAL_VENTRAL_PATTERN_FORMATION | 68 | 1.7060037 | 0.0 | 0.18497069 |
| POSITIVE_REGULATION_OF_FILOPODIUM_ASSEMBLY | 23 | 1.7044773 | 0.00589391 | 0.18069062 |
| REGULATION_OF_T_HELPER_CELL_DIFFERENTIATION | 26 | 1.6993806 | 0.0041067763 | 0.18644898 |
| SPINAL_CORD_DEVELOPMENT | 77 | 1.6937388 | 0.0 | 0.1966693 |
| POSITIVE_REGULATION_OF_INTERLEUKIN_2_PRODUCTION | 31 | 1.6870611 | 0.0020618557 | 0.20881929 |
| NEGATIVE_REGULATION_OF_DENDRITE_DEVELOPMENT | 26 | 1.6839164 | 0.0 | 0.20982039 |
| CHEMOREPELLENT_ACTIVITY | 22 | 1.6788061 | 0.004282655 | 0.22063723 |
| PROTEIN_DNA_COMPLEX_SUBUNIT_ORGANIZATION | 211 | 1.6782055 | 0.0 | 0.21470988 |
| WNT_PROTEIN_BINDING | 27 | 1.6703441 | 0.004048583 | 0.23074459 |
| REGULATION_OF_INTERLEUKIN_2_PRODUCTION | 48 | 1.6673484 | 0.0018832391 | 0.2332876 |
| EPHRIN_RECEPTOR_SIGNALING_PATHWAY | 79 | 1.666566 | 0.0 | 0.22796643 |
| SPINAL_CORD_MOTOR_NEURON_DIFFERENTIATION | 23 | 1.6634061 | 0.015414258 | 0.23156029 |
| PHOSPHATIDYLCHOLINE_BINDING | 19 | 1.6588217 | 0.0020533882 | 0.23783724 |
| HISTONE_BINDING | 166 | 1.6528594 | 0.0 | 0.24368814 |
| DORSAL_VENTRAL_AXIS_SPECIFICATION | 20 | 1.6481357 | 0.0040650405 | 0.24738972 |
| THYMOCYTE_AGGREGATION | 45 | 1.6458653 | 0.0055658626 | 0.24832642 |
| LEUKOCYTE_CELL_CELL_ADHESION | 237 | 1.6438192 | 0.0 | 0.24956845 |

NEGATIVELY REGULATED PATHWAYS

| | | | | |
|---|---|---|---|---|
| MANNOSE_BINDING | 17 | −1.7186749 | 0.0 | 0.7195255 |
| LIPID_DIGESTION | 15 | −1.7161826 | 0.0 | 0.5036717 |
| MODIFIED AMINO ACID TRANSMEMBRANE | 15 | −1.6844256 | 0.0 | 0.6472107 |
| TRANSPORTER ACTIVITY | | | | |
| RECEPTOR_AGONIST_ACTIVITY | 16 | −1.6562554 | 0.0 | 0.8093361 |
| DIGESTION | 105 | −1.6091654 | 0.0 | 0.9079605 |
| INTRINSIC COMPONENT OF ENDOPLASMIC | 117 | −1.5847611 | 0.0 | 0.8077308 |
| RETICULUM MEMBRANE | | | | |
| OXIDOREDUCTASE ACTIVITY ACTING ON THE CH_OH | 102 | −1.5729672 | 0.0 | 0.82296866 |
| GROUP OF DONORS NAD OR NADP AS ACCEPTOR | | | | |

### Detection of Biomarkers

In certain embodiments, a BCL-2 inhibitor resistance signature is detected in a subject in need thereof. In certain embodiments, the genes selected from the group consisting of: PMAIP1, BAX, NFKBIA, IKZF5, BAK1, ID3, EP300, ZEB2, NFIA, BCL2L11 and OTUD5; or FNBP1, CD9, PLXNB2, TTC39C and DENND3; or XBP1, CYBB, PAG1 and DIRAS1; or CD9, PLXNB2, TTC39C, DENND3, ICAM1, GNG7, ID2, FNBP1, FBP1, ACY3, CDKN1A, GALM, PTK2 and CYBB, are downregulated as compared to a reference value. In certain embodiments, the genes selected from the group consisting of: BCL2L1, BCL2L2, BCL2, MCL1, SRPX, RNF26, HSPB9, OR1S2, ADIPOQ, PIGF, CSGALNACT1, OTUD6A, SLC25A3, PRKAR2B, DNM2, SPHAR, APOBEC3C, RPL17, INMT, THADA, SBNO2, PRKAA2, BRMS1L, TRNAU1AP, CNNM3, ADAM33, PRKD2, FCHSD2, LOC399886, BABAM1, C1orf146, LMAN2L, ZNF460, TEX2, YRDC, ARHGAP11A, SPEG, FBXO9, USP54, SLC22A6, RPS4Y1, FAM71C, SH3BGRL2, HCRTR1, BST1, PH1F10, UCKL1, ATG5, RPS15A, CDC20B, PPIE, TUT1, RPL36, HSD11B1L, MTERF4, PTS, S1PR4, HJURP, HMMR, BOLA2, DNASE1L1, OSGEP, TMBIM4, BTNL3, CHRM3, FBX015, KLK8, ASPN, STYK1 and SRSF6; or SYT11, PARM1, ROBO2, CD48, FCRL1 and MCL1; or PLCL2, KCNA3, TNFRSF21, CYP2U1, TRAM2 and RAPGEF5; or TSTD1, DNAJC12, TRAF3IP3, OPTN, DOCK10, PYHINI, CD48, P4HA2, PLCL2, AOX1, CDK6, GATM, GLUL, PAPSS1, MCL1 and GATM, are upregulated as compared to a reference value. In certain embodiments, if a BCL-2 inhibitor resistance signature is detected, the subject may require a treatment that includes a combination therapy described herein or a therapy according to any embodiment herein that includes more than a BCL-2 inhibitor or an alternative to a BCL-2 inhibitor.

The signatures herein provide biomarkers (e.g., phenotype specific or cell type) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging a cellular response, such as an apoptotic response, in a subject by detecting a first level of expression, activity and/or function of one or more biomarkers and comparing the detected level to a control level wherein a difference in the detected level and the control level indicates that the presence of an immune response in the subject.

The biomarkers of the present invention are useful in methods of identifying patient populations at risk or suffering from resistance to cancer treatments based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The biomarkers may be used to predict disease progression. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Hence, the methods may rely on comparing the quantity of biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterized by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value > second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises 40%, 50%, ≥60%, ≥70%, ≥75% or 80% or 85% or 90% or 95% or even 100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq, quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. Detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### RNA Sequencing

In certain embodiments, the invention involves targeted nucleic acid profiling (e.g., sequencing, quantitative reverse transcription polymerase chain reaction, and the like). In certain embodiments, a target nucleic acid molecule (e.g., RNA molecule), may be sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

An altered expression of one or more genome sequences associated with a signaling biochemical pathway can be determined by assaying for a difference in the mRNA levels of the corresponding genes between the test model cell and a control cell, when they are contacted with a candidate agent. Alternatively, the differential expression of the sequences associated with a signaling biochemical pathway is determined by detecting a difference in the level of the encoded polypeptide or gene product.

To assay for an agent-induced alteration in the level of mRNA transcripts or corresponding polynucleotides, nucleic acid contained in a sample is first extracted according to standard methods in the art. For instance, mRNA can be isolated using various lytic enzymes or chemical solutions according to the procedures set forth in Sambrook et al. (1989), or extracted by nucleic-acid-binding resins following the accompanying instructions provided by the manufacturers. The mRNA contained in the extracted nucleic acid sample is then detected by amplification procedures or conventional hybridization assays (e.g. Northern blot analysis) according to methods widely known in the art or based on the methods exemplified herein.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

An agent-induced change in expression of sequences associated with a signaling biochemical pathway can also be determined by examining the corresponding gene products. Determining the protein level typically involves a) contacting the protein contained in a biological sample with an agent that specifically bind to a protein associated with a signaling biochemical pathway; and (b) identifying any agent:protein complex so formed. In one aspect of this embodiment, the agent that specifically binds a protein associated with a signaling biochemical pathway is an antibody, preferably a monoclonal antibody. The reaction is performed by contacting the agent with a sample of the proteins associated with a signaling biochemical pathway derived from the test samples under conditions that will allow a complex to form between the agent and the proteins associated with a signaling biochemical pathway. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is preferable that the label does not interfere with the binding reaction. In the alternative, an indirect detection procedure may use an agent that contains a label introduced either chemically or enzymatically. A desirable label generally does not interfere with binding or the stability of the resulting agent:polypeptide complex. However, the label is typically designed to be accessible to an antibody for an effective binding and hence generating a detectable signal.

A wide variety of labels suitable for detecting protein levels are known in the art. Non-limiting examples include radioisotopes, enzymes, colloidal metals, fluorescent compounds, bioluminescent compounds, and chemiluminescent compounds.

The amount of agent:polypeptide complexes formed during the binding reaction can be quantified by standard quantitative assays. As illustrated above, the formation of agent:polypeptide complex can be measured directly by the amount of label remained at the site of binding. In an alternative, the protein associated with a signaling biochemical pathway is tested for its ability to compete with a labeled analog for binding sites on the specific agent. In this competitive assay, the amount of label captured is inversely proportional to the amount of protein sequences associated with a signaling biochemical pathway present in a test sample

A number of techniques for protein analysis based on the general principles outlined above are available in the art. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Antibodies that specifically recognize or bind to proteins associated with a signaling biochemical pathway are preferable for conducting the aforementioned protein analyses. Where desired, antibodies that recognize a specific type of post-translational modifications (e.g., signaling biochemical pathway inducible modifications) can be used. Post-translational modifications include but are not limited to glycosylation, lipidation, acetylation, and phosphorylation. These antibodies may be purchased from commercial vendors. For example, anti-phosphotyrosine antibodies that specifically recognize tyrosine-phosphorylated proteins are available from a number of vendors including Invitrogen and Perkin Elmer. Antiphosphotyrosine antibodies are particularly useful in detecting proteins that are differentially phosphorylated on their tyrosine residues in response to an ER stress. Such proteins include but are not limited to eukaryotic translation initiation factor 2 alpha. Alternatively, these antibodies can be generated using conventional polyclonal or monoclonal antibody technologies by immunizing a host animal or an antibody-producing cell with a target protein that exhibits the desired post-translational modification.

In practicing the subject method, it may be desirable to discern the expression pattern of an protein associated with a signaling biochemical pathway in different bodily tissue, in different cell types, and/or in different subcellular structures. These studies can be performed with the use of tissue-specific, cell-specific or subcellular structure specific antibodies capable of binding to protein markers that are preferentially expressed in certain tissues, cell types, or subcellular structures.

An altered expression of a gene associated with a signaling biochemical pathway can also be determined by examining a change in activity of the gene product relative to a control cell. The assay for an agent-induced change in the activity of a protein associated with a signaling biochemical pathway will dependent on the biological activity and/or the signal transduction pathway that is under investigation. For example, where the protein is a kinase, a change in its ability to phosphorylate the downstream substrate(s) can be determined by a variety of assays known in the art. Representative assays include but are not limited to immunoblotting and immunoprecipitation with antibodies such as anti-phosphotyrosine antibodies that recognize phosphorylated proteins. In addition, kinase activity can be detected by high throughput chemiluminescent assays such as AlphaScreen™ (available from Perkin Elmer) and eTag™ assay (Chan-Hui, et al. (2003) Clinical Immunology 111: 162-174).

Where the protein associated with a signaling biochemical pathway is part of a signaling cascade leading to a fluctuation of intracellular pH condition, pH sensitive molecules such as fluorescent pH dyes can be used as the reporter molecules. In another example where the protein associated with a signaling biochemical pathway is an ion channel, fluctuations in membrane potential and/or intracellular ion concentration can be monitored. A number of commercial kits and high-throughput devices are particularly suited for a rapid and robust screening for modulators of ion channels. Representative instruments include FLIPR™ (Molecular Devices, Inc.) and VIPR (Aurora Biosciences). These instruments are capable of detecting reactions in over 1000 sample wells of a microplate simultaneously, and providing real-time measurement and functional data within a second or even a minisecond.

### Systems and Devices

The systems described herein can be embodied on diagnostic devices. A number of substrates and configurations may be used. The devices may be capable of defining multiple individual discrete volumes within the device. As used herein an “individual discrete volume” refers to a discrete space, such as a container, receptacle, or other defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof that can contain a sample within a defined space. Individual discrete volumes may be identified by molecular tags, such as nucleic acid barcodes. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain embodiments, the compartment is an aqueous droplet in a water-in-oil emulsion. In specific embodiments, any of the applications, methods, or systems described herein requiring exact or uniform volumes may employ the use of an acoustic liquid dispenser.

In certain example embodiments, the device comprises a flexible material substrate on which a number of spots may be defined. Flexible substrate materials suitable for use in diagnostics and biosensing are known within the art. The flexible substrate materials may be made of plant derived fibers, such as cellulosic fibers, or may be made from flexible polymers such as flexible polyester films and other polymer types. Within each defined spot, reagents of the system described herein are applied to the individual spots. Each spot may contain the same reagents except for a different guide RNA or set of guide RNAs, or where applicable, a different detection aptamer to screen for multiple targets at once. Thus, the systems and devices herein may be able to screen samples from multiple sources (e.g. multiple clinical samples from different individuals) for the presence of the same target, or a limited number of targets, or aliquots of a single sample (or multiple samples from the same source) for the presence of multiple different targets in the sample. In certain example embodiments, the elements of the systems described herein are freeze dried onto the paper or cloth substrate. Example flexible material based substrates that may be used in certain example devices are disclosed in Pardee et al. Cell. 2016, 165(5):1255-66 and Pardee et al. Cell. 2014, 159(4):950-54. Suitable flexible material-based substrates for use with biological fluids, including blood are disclosed in International Patent Application Publication No. WO/2013/071301 entitled “Paper based diagnostic test” to Shevkoplyas et al. U.S. Patent Application Publication No. 2011/0111517 entitled “Paper-based microfluidic systems” to Siegel et al. and Shafiee et al. “Paper and Flexible Substrates as Materials for Biosensing Platforms to Detect Multiple Biotargets” Scientific Reports 5:8719 (2015). Further flexible based materials, including those suitable for use in wearable diagnostic devices are disclosed in Wang et al. “Flexible Substrate-Based Devices for Point-of-Care Diagnostics” Cell 34(11):909-21 (2016). Further flexible based materials may include nitrocellulose, polycarbonate, methylethyl cellulose, polyvinylidene fluoride (PVDF), polystyrene, or glass (see e.g., US20120238008). In certain embodiments, discrete volumes are separated by a hydrophobic surface, such as but not limited to wax, photoresist, or solid ink.

In other example embodiments, the elements of the systems described herein may be place on a single use substrate, such as swab or cloth that is used to swab a surface or sample fluid. For example, the system could be used to test for the presence of a pathogen on a food by swabbing the surface of a food product, such as a fruit or vegetable. Similarly, the single use substrate may be used to swab other surfaces for detection of certain microbes or agents, such as for use in security screening. Single use substrates may also have applications in forensics, where the CRISPR systems are designed to detect, for example identifying DNA SNPs that may be used to identify a suspect, or certain tissue or cell markers to determine the type of biological matter present in a sample. Likewise, the single use substrate could be used to collect a sample from a patient—such as a saliva sample from the mouth—or a swab of the skin. In other embodiments, a sample or swab may be taken of a meat product on order to detect the presence of absence of contaminants on or within the meat product.

In certain example embodiments, a single guide sequences specific to a single target is placed in separate volumes. Each volume may then receive a different sample or aliquot of the same sample. In certain example embodiments, multiple guide sequences each to separate target may be placed in a single well such that multiple targets may be screened in a different well. In order to detect multiple guide RNAs in a single volume, in certain example embodiments, multiple effector proteins with different specificities may be used. For example, different orthologs with different sequence specificities may be used. For example, one orthologue may preferentially cut A, while others preferentially cut C, G, U/T. Accordingly, masking constructs completely comprising, or comprised of a substantial portion, of a single nucleotide may be generated, each with a different fluorophore that can be detected at differing wavelengths. In this way, up to four different targets may be screened in a single individual discrete volume. In certain example embodiments, different orthologues from a same class of CRISPR effector protein may be used, such as two Cas13a orthologues, two Cas13b orthologues, or two Cas13c orthologues. The nucleotide preferences of various Cas13 proteins is shown in FIGS. 67A and 67B. In certain other example embodiments, different orthologues with different nucleotide editing preferences may be used such as a Cas13a and Cas13b orthologs, or a Cas13a and a Cas13c orthologs, or a Cas13b orthologs and a Cas13c orthologs etc. In certain example embodiments, a Cas13 protein with a polyU preference and a Cas13 protein with a polyA preference are used. In certain example embodiments, the Cas13 protein with a polyU preference is a Prevotella intermedia Cas13b. and the Cas13 protein with a polyA preference is a Prevotella sp. MA2106 Cas13b protein (PsmCas13b). In certain example embodiments, the Cas13 protein with a polyU preference is a Leptotrichia wadei Cas13a (LwaCas13a) protein and the Cas13 protein with a poly A preference is a Prevotella sp. MA2106 Cas13b protein. In certain example embodiments, the Cas13 protein with a polyU preference is Capnocytophaga canimorsus Cas13b protein (CcaCas13b).

In certain embodiments, the systems, methods, and devices described herein may be used to screen gene signatures that identify a particular cell type, cell phenotype, or cell state. Likewise, through the use of such methods as compressed sensing, the embodiments disclosed herein may be used to detect transcriptomes. Gene expression data are highly structured, such that the expression level of some genes is predictive of the expression level of others. Knowledge that gene expression data are highly structured allows for the assumption that the number of degrees of freedom in the system are small, which allows for assuming that the basis for computation of the relative gene abundances is sparse. It is possible to make several biologically motivated assumptions that allow Applicants to recover the nonlinear interaction terms while under-sampling without having any specific knowledge of which genes are likely to interact. In particular, if Applicants assume that genetic interactions are low rank, sparse, or a combination of these, then the true number of degrees of freedom is small relative to the complete combinatorial expansion, which enables Applicants to infer the full nonlinear landscape with a relatively small number of perturbations. Working around these assumptions, analytical theories of matrix completion and compressed sensing may be used to design under-sampled combinatorial perturbation experiments. In addition, a kernel-learning framework may be used to employ under-sampling by building predictive functions of combinatorial perturbations without directly learning any individual interaction coefficient Compresses sensing provides a way to identify the minimal number of target transcripts to be detected in order obtain a comprehensive gene-expression profile. Methods for compressed sensing are disclosed in PCT/US2016/059230 “Systems and Methods for Determining Relative Abundances of Biomolecules” filed Oct. 27, 2016, which is incorporated herein by reference. Having used methods like compressed sensing to identify a minimal transcript target set, a set of corresponding guide RNAs may then be designed to detect said transcripts. Accordingly, in certain example embodiments, a method for obtaining a gene-expression profile of cell comprises detecting, using the embodiments disclosed, herein a minimal transcript set that provides a gene-expression profile of a cell or population of cells.

### Therapeutic Methods

In certain embodiments, the identified signatures, biomarkers and pathways described herein are modulated in order to treat a subject in need thereof, such as a subject suffering from cancer (e.g., a lymphoma). In one aspect, the present invention provides for a method of inhibiting tumor growth of a BCL-2-driven cancer in a subject in need thereof, the method comprising administering to said subject a therapeutically effective amount of one or more agents that induces or enhances expression, activity, and/or function of one or more BCL-2 inhibitor resistance signature genes (see, Tables 1, 2, 3 and 4). In certain embodiments, a resistant signature is shifted to a sensitive signature. In certain embodiments, a combination treatment is administered in order to overcome resistance to the primary treatment (e.g., a BCL-2 inhibitor in combination with an MCL1 inhibitor, ID2 or ID3 agonist, or OXPHOS inhibitor).

The term “treat”, “treated,” “treating” or “treatment” is used herein to mean to relieve, reduce or alleviate at least one symptom of a disease in a subject. For example, treatment can be diminishment of one or several symptoms of a disorder or complete eradication of a disorder, such as cancer. Within the meaning of the present invention, the term “treat” also denote to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject. Within the meaning of the present invention, the disease is associated with a cancer.

The term “subject” or “patient” is intended to include animals, which are capable of suffering from or afflicted with a cancer or any disorder involving, directly or indirectly, a cancer. Examples of subjects include mammals, e.g., humans, dogs, cows, horses, pigs, sheep, goats, cats, mice, rabbits, rats, and transgenic non-human animals. In certain embodiments, the subject is a human, e.g., a human having, at risk of having, or potentially capable of having cancer.

The methods described herein may be applicable to the treatment, diagnosis, or prognosis of any cancer. The term “cancer” is used herein to mean malignant solid tumors as well as hematological malignancies. In some instances, the cancer is melanoma. The melanoma may be metastatic melanoma. Additional examples of such tumors include but are not limited to leukemias, lymphomas, myelomas, carcinomas, metastatic carcinomas, sarcomas, adenomas, nervous system cancers and genitourinary cancers. In exemplary embodiments, the foregoing methods are useful in treating adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, osteosarcoma, fibrous histiocytoma, brain cancer, brain stem glioma, cerebellar astrocytoma, malignant glioma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinema, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, and Wilms tumor.

### Therapeutic Agents

In certain embodiments, the present invention provides for one or more therapeutic agents against single or combinations of targets identified. Targeting the identified combinations may provide for enhanced or otherwise previously unknown activity in the treatment of disease. In certain embodiments, an agent against one of the targets in a combination may already be known or used clinically. In certain embodiments, targeting the combination may require less of the agent as compared to the current standard of care and provide for less toxicity and improved treatment. In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In one aspect, the present invention provides for a method of inhibiting tumor growth of a BCL-2-driven cancer in a subject in need thereof comprising administering to the subject one or more agents capable of inhibiting the oxidative phosphorylation system (OXPHOS). In certain embodiments, the method comprises administering to the subject a combination therapy comprising an inhibitor of BCL-2 and one or more inhibitors selected from the group consisting of an AMPK inhibitor and mitochondrial electron transport chain (mETC) inhibitor.

In another aspect, the present invention provides for a method of inhibiting tumor growth of a BCL-2-driven cancer in a subject in need thereof comprising administering to the subject a combination therapy comprising an inhibitor of BCL-2 and one or more NF kappa B inhibitors.

### BCL-2

In certain embodiments, the present invention uses inhibitors of BCL-2 to modulate BCL-2 driven tumors. Targeted and selective BCL-2 inhibitors include, but are not limited to, antisense oligonucleotide drugs such as oblimersen, small molecule inhibitors such as ABT-737 and navitoclax (ABT-263) and mimetic drugs such as venetoclax (ABT-199).

Bcl-2 has been shown to interact with: BAK1, BCAP31, BCL2-like 1, BCL2L11, BECN1, BID, BMF, BNIP2, BNIP3, BNIPL, BAD, BAX, BIK, C-Raf, CAPN2, CASP8, Cdk1, HRK, IRS1, Myc, NR4A1, Noxa, PPP2CA, PSEN1, RAD9A, RRAS, RTN4, SMN1, SOD1, and TP53BP2.

Venetoclax resistance modulating agents are useful therapeutic tools in cancers, as BCL-2 has been implicated in these indications. Unlike oncogenes that promote uncontrolled cellular proliferation, BCL-2 encodes an anti-apoptotic protein that inhibits cell death. Venetoclax, previously known as ABT-199 is the first FDA-approved treatment that targets the B-cell lymphoma 2 (BCL-2) protein. The BCL-2 protein plays an important role in enabling CLL cells to survive.

BCL-2 plays a role in many tumor types. BCL-2 was first discovered as an oncogene in B-cell malignancies. It is also expressed in normal lymphoid cells including T-cells and BCL-2 inhibitors are useful for treatment. Accordingly, venetoclax resistance modulating agents are used to treat B-cell and T-cell malignancies. Moreover, the venetoclax resistance modulating agents are used more generally in BCL-2 driven cancers with other BCL-2 inhibitors when resistance develops to those inhibitors. BCL-2 inhibitors includes, without limitation, navitoclax (ABT-263), obatoclax (GX15-070), and gossypol compounds. (See e.g., Lampson et al., “The Development and Current Use of BCL-2 Inhibitors for the Treatment of Chronic Lymphocytic Leukemia.” Curr Hematol Malig Rep. 2017 February; 12(1): 11-19. doi:10.1007/s11899-017-0359-0). Furthermore, the resistance modulating agents are more generally used in combination with BCL-2 inhibitors at a stage where resistance has not developed. Moreover, the agents can be used with BCL-2 inhibitors in cancers that otherwise are not responsive to BCL-2 inhibition.

BCL2 is expressed in non-lymphoid cells and has been described in neuronal tumors. (See, e.g., I. Garcia, I. Martinou, Y. Tsujimoto, and J. Martinou, “Prevention of programmed cell death of sympathetic neurons by the bcl-2 proto-oncogene,” Science, vol. 258, no. 5080, pp. 302-304, 1992.) Accordingly, resistance modulating agents according to the invention can be combined with BCL2 inhibitors, more generally BCL2-family inhibitors for treatment of such tumor types.

Expression of BCL2 in normal epithelium indicates that BCL2 is expressed in carcinoma. For example, high expression of BCL2 is found in prostate cancer, including in androgen-independent tumors. (See, e.g., T. J. McDonnell, P. Troncoso, S. M. Brisbay et al., “Expression of the protooncogene bcl-2 in the prostate and its association with emergence of androgen-independent prostate cancer,” Cancer Research, vol. 52, no. 24, pp. 6940-6944, 1992.)

High BCL2 expression has been reported in many different tumor types including non-small cell and small lcell lung cancer (See, e.g., F. Pezzella, H. Turley, I. Kuzu et al., “bcl-2 protein in non-small-cell lung carcinoma,” The New England Journal of Medicine, vol. 329, no. 10, pp. 690-694, 1993; N. Ikegaki, M. Katsumata, J. Minna, and Y. Tsujimoto, “Expression of bcl-2 in small cell lung carcinoma cells,” Cancer Research, vol. 54, no. 1, pp. 6-8, 1994.) BCL-2 expression is observed in ovarian cancer (See, e.g., Y. Kuwashima, T. Uehara, K. Kishi, K. Shiromizu, M. Matsuzawa, and S. Takayama, “Immunohistochemical characterization of undifferentiated carcinomas of the ovary,” Journal of Cancer Research and Clinical Oncology, vol. 120, no. 11, pp. 672-677, 1994.) and breast cancer (See, e.g., P. Monaghan, D. Robertson, T. A. S. Amos, M. J. S. Dyer, D. Y. Mason, and M. F. Greaves, “Ultrastructural localization of BCL-2 protein,” Journal of Histochemistry and Cytochemistry, vol. 40, no. 12, pp. 1819-1825, 1992.). The function of BCL2 in inhibiting apoptosis has been proven in many independent studies, for example, by overexpression or knockdown. (See, e.g., R. X.-D. Song, Z. Zhang, G. Mor, and R. J. Santen, “Down-regulation of Bcl-2 enhances estrogen apoptotic action in long-term estradiol-depleted ER⁺ breast cancer cells,” Apoptosis, vol. 10, no. 3, pp. 667-678, 2005.) Accordingly, resistance modulating agents according to the invention can be combined with BCL2 inhibitors, more generally BCL2-family inhibitors for treatment of such tumor types. In certain such embodiments, the contributions of the BCL-2 or BCL-2 family inhibitor and resistance modulating agents are additive. In other embodiments, the contributions are synergistic. In certain embodiments, the resistance modulating agent effects or enables the action of the BCL-2 or BCL-2 family inhibitor, i.e. the effect of the inhibitor is observed when the resistance modulating agent is present.

NF kappa B (NF-κB)

In certain embodiments, the present invention uses inhibitors of NF kappa B to modulate BCL-2 driven tumors. As discussed further herein, NKBIA was identified in the loss-of-function screen for BCL-2 inhibitor resistance. Thus, loss of an inhibitor of NF kappa B provided for resistance to BCL-2 inhibition. Protein inhibitors of NF kappa B activity include, but are not limited to, IFRD1 and SIRT1. Other drugs that inhibit NF kappa B activity include, but are not limited to, denosumab, disulfiram, olmesartan, dithiocarbamates, anatabine, BAY 11-7082 and iguratimod. In certain embodiments, a combination therapy comprising an NF kappa B inhibitor and BCL-2 inhibitor is used to treat a subject in need thereof.

### Oxidative Phosphorylation

In certain embodiments, the present invention uses inhibitors of oxidative phosphorylation to modulate BCL-2 driven tumors. OXPHOS Inhibitors for use in treating cancer have been described and are applicable to the present invention (see, e.g., Nayak et al., Oxidative Phosphorylation: A Target for Novel Therapeutic Strategies Against Ovarian Cancer. Cancers (Basel). 2018 September; 10(9): 337). In certain embodiments, inhibitors of oxidative phosphorylation include, but are not limited to biguanides, atovaquone, plumbagin, thiazolidinediones and ubiquinone. Complex I Biguanides include metformin, proguanil, and IACS-0107059. Thiazolidinediones include rosiglitazone. Dorsomorphin is a cell-permeable and reversible ATP-competitive inhibitor of AMP-activated protein kinase (AMPK) with Ki value of 10⁹ nM (see, e.g., Lu Y, Akinwumi B C, Shao Z, Anderson H D. Ligand Activation of Cannabinoid Receptors Attenuates Hypertrophy of Neonatal Rat Cardiomyocytes. J Cardiovasc Pharmacol. 2014 Jun. 26). Oligomycin is a specific inhibitor of the ATPase and blocks proton translocation leading to a hyperpolarization of the inner mitochondrial membrane. Antimycin A is an inhibitor of cellular respiration, specifically oxidative phosphorylation. Antimycin A binds to the Qi site of cytochrome c reductase, inhibiting the oxidation of ubiquinone in the Qi site of ubiquinol thereby disrupting the Q-cycle of enzyme turn over.

### MCL-1

Myeloid cell leukemia-1 (MCL-1), a member of anti-apoptotic BCL-2 family proteins, is a key regulator of mitochondrial homeostasis and is frequently overexpressed in human primary and drug-resistant cancer cells (see, e.g., Xiang et al., MCL-1 inhibition in cancer treatment. Onco Targets Ther. 2018; 11: 7301-7314). Non-limiting inhibitors include AT-101, TW-37, GA, Sabutoclax (BI-97C1), maritoclax, UMI-77, A-1210477, MIK665/S64315 and S63845, AMG176, and AZD5991. Previous studies have described venetoclax and MCL-1 inhibition (see, e.g., Luedtke et al., Inhibition of Mcl-1 enhances cell death induced by the Bcl-2-selective inhibitor ABT-199 in acute myeloid leukemia cells. Signal Transduct Target Ther. 2017; 2( ):17012).

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810).

### Genetic Modifying Agents

In certain embodiments, the one or more modulating agents may be a genetic modifying agent. The genetic modifying agent may comprise a CRISPR system, a zinc finger nuclease system, a TALEN, a meganuclease or RNAi system.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein His A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellect is directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of Orthologous proteins may but need not be structurally related, or are only partially structurally related.

### Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2d/or non-naturally occurring nucleotides and/or nucleotide analogs, and/or chemically modifications. Preferably, these non-naturally occurring nucleic acids and nouoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2ccurrinhyl (M), 2′-O-methyl 3′phosphorothioate (MS), S-constrained ethyl(cEt), or 2ained ethyl (cEtxamples of guide RNA chemical modifications include, without limitation, incorporation of 2ccurrinhyl (M), 2′-O-methyl 3′phosphorothioate (MS), r chemically modificauides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med Chem. 2005, 48:901-904; Bramsen et al., Front. Genet., 2012, 3:154; Deng et al., PNAS, 2015, 112:11870-11875; Sharma et al., Med Chem Comm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine (5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the compelementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans;4/164/rs2), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (40HT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogren receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 .mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extented length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### CRISPR RNA-Targeting Effector Proteins

In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprise one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease (also referred to as Cas13a). The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

In certain embodiments, the C2c2 effector protein is from an organism of a genus selected from the group consisting of: Leptotrichia, Listeria, Corynebacterium, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia. wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYLL. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

### Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytindine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g, Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present invention may also use a Cas12 CRISPR enzyme. Cas12 enzymes include Cas12a (Cpf1), Cas12b (C2c1), and Cas12c (C2c3), described further herein. The Cas12 may be an ultraCas12. IDT developed a “Alt-R Cas12a” reagent that has 3 main components: a) optimized crRNA; b) A.s. Cas12a; and (c) an electroporation enhancer (for better transfection). The variant is an improved version of IDT's Alt-R Cas12a and is named “Alt-R Cas12a Ultra.”

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 August 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh 00, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91(2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpf1 Enzymes with Altered PAM Specificities,” bioRxiv 091611; doi: http://dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.
Gaudelli et al. “Programmable base editing of A-T to G-C in genomic DNA without DNA cleavage” Nature 464(551); 464-471 (2017).
Strecker et al., “Engineering of CRISPR-Cas12b for human genome editing,” Nature Communications volume 10, Article number: 212 (2019).

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.
Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

Also, Harrington et al. “Programmed DNA destruction by miniature CRISPR-Cas14 enzymes” Science 2018 doi:10/1126/science.aav4293, relates to Cas14.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly these polypeptides can be used for the delivery of CRISPR-effector based RNPs in eukaryotic cells.

### Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

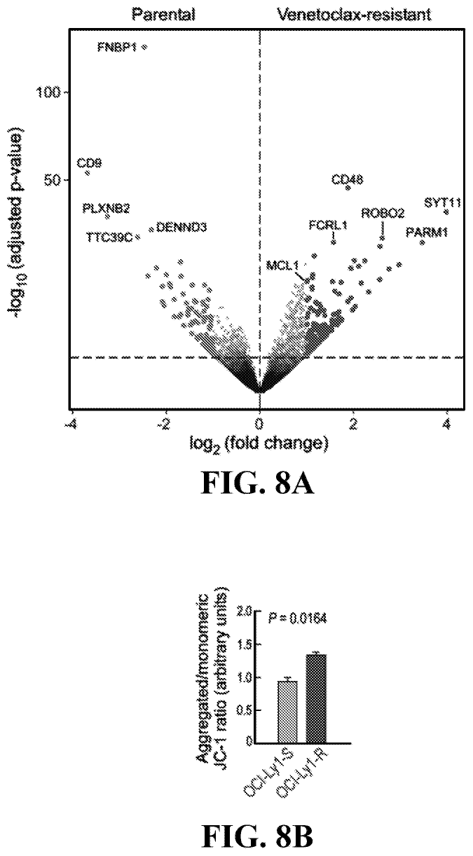

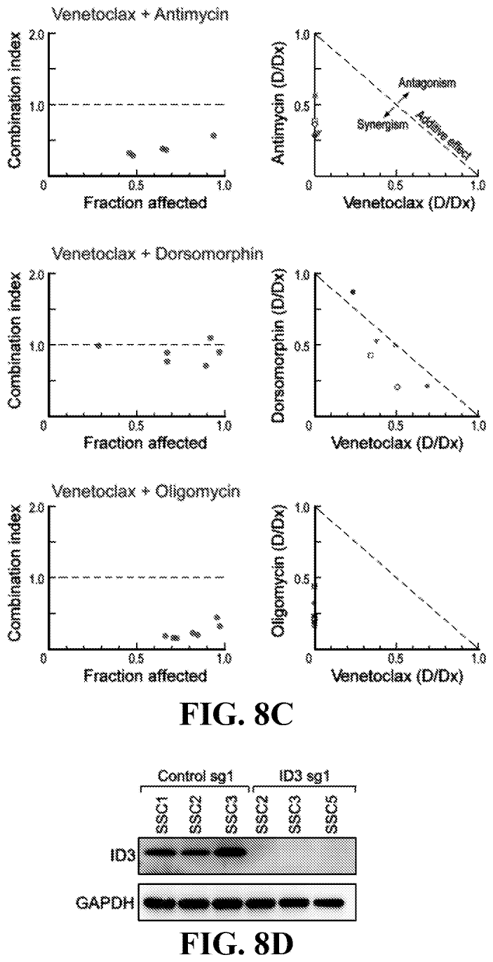

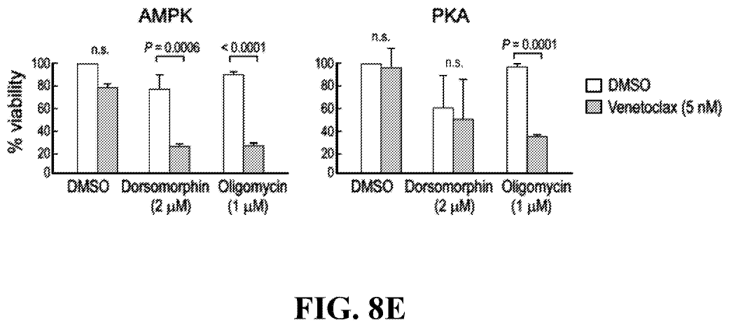

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID No. 3)
M D P I R S R T P S P A R E L L S G P Q P D G V Q P
T A D R G V S P P A G G P L D G L P A R R T M S R T
R L P S P P A P S P A F S A D S F S D L L R F D P S
L F N T S L F D S L P P F G A H H T E A A T G E W D
E V Q S G L R A A D A P P P T M R V A V T A A R P P
R A K P A P R R R A A Q P S D A S P A A Q V D L R T
L G Y S Q Q Q Q E K I K P K V R S T V A Q H H E A L
V G H G F T H A H I V A L S Q H P A A L G T V A V K
Y Q D M I A A L P E A T H E A I V G V G K Q W S G A
R A L E A L L T V A G E L R G P P L Q L D T G Q L L
K I A K R G G V T A V E A V H A W R N A L T G A P L
N

An exemplary amino acid sequence of a C-terminal capping region is:

(SEQ ID No. 4)
R P A L E S I V A Q L S R P D P A L A A L T N D H L
V A L A C L G G R P A L D A V K K G L P H A P A L I
K R T N R R I P E R T S H R V A D H A Q V V R V L G
F F Q C H S H P A Q A F D D A M T Q F G M S R H G L
L Q L F R R V G V T E L E A R S G T L P P A S Q R W
D R I L Q A S G M K R A K P S P T S T Q T P D Q A S
L H A F A D S L E R D L D A P S P M H E G D Q T R A
S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### ZN-Finger Nucleases

Other preferred tools for genome editing for use in the context of this invention include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Antibodies

In certain embodiments, the one or more agents is an antibody. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by p pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 M or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Aptamers

In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH2), 2′-fluoro (2′-F), and/or 2′-O-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, 0-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colo.). In certain embodiments, the present invention may utilize any aptamer containing any modification as described herein.

### Administration and Pharmaceutical Agents

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2): 1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Techno 1. 52:238-311 (1998) and the citations therein for additional information related to formulations, excipients and carriers well known to pharmaceutical chemists. In practicing any of the methods disclosed herein, a suitable vector can be introduced to a cell or an embryo via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the vector is introduced into an embryo by microinjection. The vector or vectors may be microinjected into the nucleus or the cytoplasm of the embryo. In some methods, the vector or vectors may be introduced into a cell by nucleofection. Provided herein are pharmaceutical formulations comprising single agents, such as BCL-2 inhibitors, NF kappa B inhibitors, AMPK inhibitors and/or mitochondrial electron transport chain (mETC) inhibitors (and/or pharmacologically active metabolites, salts, solvates and racemates thereof).

Agents may contain one or more asymmetric elements such as stereogenic centers or stereogenic axes, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms; all isomeric forms of the compounds are included in the present invention. In these situations, the single enantiomers (optically active forms) can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

Unless otherwise specified, or clearly indicated by the text, reference to compounds useful in the therapeutic methods of the invention includes both the free base of the compounds, and all pharmaceutically acceptable salts of the compounds. The term “pharmaceutically acceptable salts” includes derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, and nitric; other acceptable inorganic salts include metal salts such as sodium salt, potassium salt, and cesium salt; and alkaline earth metal salts, such as calcium salt and magnesium salt; and combinations comprising one or more of the foregoing salts. In some embodiments, the salt is a hydrochloride salt.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC(CH.sub.2).sub.nCOOH where n is 0-4; organic amine salts such as triethylamine salt, pyridine salt, picoline salt, ethanolamine salt, triethanolamine salt, dicyclohexylamine salt, N,N′-dibenzylethylenediamine salt; and amino acid salts such as arginate, asparginate, and glutamate, and combinations comprising one or more of the foregoing salts.

The agents of the invention are administered in effective amounts. An “effective amount” is an amount sufficient to provide an observable improvement over the baseline clinically observable signs and symptoms of the disorder treated with the combination.

The effective amount may be determined using known methods and will depend upon a variety of factors, including the activity of the agents; the age, body weight, general health, gender and diet of the subject; the time and route of administration; and other medications the subject is taking. Effective amounts may be established using routine testing and procedures that are well known in the art.

A physician or veterinarian having ordinary skill in the art can readily determine and prescribe the effective amount of the pharmaceutical composition required. For example, the physician or veterinarian could start at doses lower than those required to achieve the desired therapeutic effect and gradually increase the dosage until the desired effect is achieved. In general, a suitable daily dose of will be that amount of the compound that is the lowest dose effective to produce a therapeutic effect.

Generally, therapeutically effective doses of the compounds of this invention for a patient will range from about 0.0001 to about 1000 mg per kilogram of body weight per day, more preferably from about 0.01 to about 50 mg per kg per day.

If desired, the effective daily dose of the active compound may be administered as two, three, four, five, six or more sub-doses administered separately at appropriate intervals throughout the day, optionally, in unit dosage forms.

The agents may be administered using a variety of routes of administration known to those skilled in the art. The agents may be administered to humans and other animals orally, parenterally, sublingually, by aerosolization or inhalation spray, rectally, intracisternally, intravaginally, intraperitoneally, bucally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agents as compared to the other agent in the combination. Therefore, to permit appropriate dosing, packaged pharmaceutical products may contain one or more dosage forms that contain the combination of agents, and one or more dosage forms that contain one of the combinations of agents, but not the other agent(s) of the combination. Administration may be concurrent or sequential.

The pharmaceutical formulations may additionally comprise a carrier or excipient, stabilizer, flavoring agent, and/or coloring agent. Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono or di glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

The pharmaceutical products can be released in various forms. “Releasable form” is meant to include instant release, immediate-release, controlled-release, and sustained-release forms.

“Instant-release” is meant to include a dosage form designed to ensure rapid dissolution of the active agent by modifying the normal crystal form of the active agent to obtain a more rapid dissolution.

“Immediate-release” is meant to include a conventional or non-modified release form in which greater than or equal to about 50% or more preferably about 75% of the active agents is released within two hours of administration, preferably within one hour of administration.

“Sustained-release” or “extended-release” includes the release of active agents at such a rate that blood (e.g., plasma) levels are maintained within a therapeutic range but below toxic levels for at least about 8 hours, preferably at least about 12 hours, more preferably about 24 hours after administration at steady-state. The term “steady-state” means that a plasma level for a given active agent or combination of active agents, has been achieved and which is maintained with subsequent doses of the active agent(s) at a level which is at or above the minimum effective therapeutic level and is below the minimum toxic plasma level for a given active agent(s).

The pharmaceutical products can be administrated by oral dosage form. “Oral dosage form” is meant to include a unit dosage form prescribed or intended for oral administration. An oral dosage form may or may not comprise a plurality of subunits such as, for example, microcapsules or microtablets, packaged for administration in a single dose.

Compositions for rectal or vaginal administration are preferably suppositories which can be prepared by mixing the compounds of this invention with suitable non irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles.

Aerosolized formulations of the invention may be delivered using an aerosol forming device, such as a jet, vibrating porous plate or ultrasonic nebulizer, preferably selected to allow the formation of an aerosol particles having with a mass medium average diameter predominantly between 1 to 5 microns. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1 to 5 microns. Predominantly in this application means that at least 70% but preferably more than 90% of all generated aerosol particles are within 1 to 5 micron range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. The compounds of the present invention can also be administered in the form of liposomes. As is known in the art, liposomes are generally derived from phospholipids or other lipid substances. Liposomes are formed by mono or multi lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

### Screening Methods

### Modulating Agents

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more phenotypic aspects of the cell or cell population by the candidate agent, thereby identifying the agent. The phenotypic aspects of the cell or cell population that is modulated may be a gene signature, biomarker or pathway specific to a cell type or cell phenotype or phenotype specific to a population of cells (e.g., a BCL-2 inhibitor resistance phenotype). In certain embodiments, steps can include administering candidate modulating agents to cells, detecting changes in signatures, or identifying relative changes in cell populations which may comprise detecting relative abundance of particular gene signatures. In certain embodiments, the one or more candidate agents increase expression, activity, and/or function of one or more BCL-2 inhibitor resistance genes or gene products. In certain embodiments, the one or more candidate agents increase expression, activity, and/or function of one or more target genes or one or more products of one or more target genes which comprise inhibitors of the NF-Kappa B pathway, lymphoid transcription factors and modulators, ubiquitination components, and/or pro-apoptotic BCL-2 family proteins. In certain embodiments, the one or more candidate agents decrease expression, activity, and/or function of one or more target genes or one or more products of one or more target genes which comprise energy-stress sensor signaling pathway components, a mitochondrial energy metabolism component, vesicle transport/autophagy components, ribosomal components, and/or ubiquitination components.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of a cell or cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

In certain embodiments, this invention provides a method of developing a biologically active agent that modulates a cell signaling event associated with a disease gene. The method comprises contacting a test compound with a cell comprising one or more vectors that drive expression of one or more of a CRISPR enzyme, and a direct repeat sequence linked to a guide sequence; and detecting a change in a readout that is indicative of a reduction or an augmentation of a cell signaling event associated with, e.g., a mutation in a disease gene contained in the cell.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on phenotypes thereof simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or pathways of the present invention may be used to screen for drugs that reduce the signature or pathway in cells as described herein. The signature or pathways may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or pathway(s) of the present invention in silico.

### Resistance Mechanisms

In certain embodiments, the invention provides methods and compositions for identifying genome-scale loss- (LOF) and gain-of-function (GOF) genetic modifiers of resistance to BCL-2 and BCL-2 family inhibitors such as but not limited to venetoclax. The invention provides screens to be performed to identify target genes and resistance mechanisms in BCL-2 family protein driven cancers. These target genes are identified, for example, by contacting a cell expressing BCL-2 or BCL-2 family protein cell, e.g., a BCL-2 driven tumor cell, with a BCL-2 inhibitor and another modulating agent and monitoring the effect on viability. These target genes In other embodiments, a cell expressing BCL-2 or BCL-2 family protein, e.g., a BCL-2 driven tumor cell is contacted with a BCL-2 inhibitor or other modulating agent and the effect, if any, on the expression of one or more signature genes or one or more products of one or more signature genes is monitored.

In certain embodiments, the present invention provides for genome-scale-loss (LOF) and gain-of-function (GOF) screens. Loss of function screens may use CRISPR systems to knockout individual genes in individual cells in a population of cells. For CRISPR methods, see, e.g. U.S. Pat. Nos. 9,840,713; 9,822,372; 9,790,490; 8,999,641; 8,993,233; 8,945,839; 8,932,814; 8,906,616; 8,895,308; 8,889,418; 8,889,356; 8,871,445; 8,865,406; 8,795,965; 8,771,945; 8,697,359 and US Patent Publication Nos. 20180274017; 20180235961; 20180171297; 20180127783; 20180068062; 20170211142; 20160338326; 20160305934; 20160282354; 20140335620; 20140287938; 20140273232; 20140273231; 20140256046; 20140248702; 20140242700; 20140242699; 20140242664; 20140234972; 20140227787; 20140186843; 20140179770; 20140179006 and 20140170753. Gain of function screens may use vectors that overexpress individual genes in individual cells in a population of cells. In certain embodiments, the screening method screens for cell viability. Cell viability may be tested for by measuring enrichment of cells comprising either guide sequences or vectors specific to a target gene. Cell viability may be tested for by measuring depletion of cells comprising either guide sequences or vectors specific to a target gene as compared to the original proportion in the initial population. Thus, targets affecting viability may be detected.

In certain embodiments, genomewide screens according to the present invention may be performed in additional cell lines, in particular cancer cell lines. In certain embodiments, the cell is derived from cells taken from a subject, such as a cell line. A wide variety of cell lines for tissue culture models are known in the art. Examples of cell lines include, but are not limited to, OCI-LY1, HT115, RPE1, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

### Kits

The present invention also comprises a kit with a detection reagent that binds to one or more signature nucleic acids. Also provided by the invention is an array of detection reagents, e.g., oligonucleotides that can bind to one or more signature nucleic acids. Suitable detection reagents include nucleic acids that specifically identify one or more signature nucleic acids by having homologous nucleic acid sequences, such as oligonucleotide sequences, complementary to a portion of the signature nucleic acids packaged together in the form of a kit. The oligonucleotides can be fragments of the signature genes. For example, the oligonucleotides can be 200, 150, 100, 50, 25, 10 or fewer nucleotides in length. The kit may contain in separate container or packaged separately with reagents for binding them to the matrix), control formulations (positive and/or negative), and/or a detectable label such as fluorescein, green fluorescent protein, rhodamine, cyanine dyes, Alexa dyes, luciferase, radio labels, among others. Instructions (e.g., written, tape, VCR, CD-ROM, etc.) for carrying out the assay may be included in the kit. The assay may for example be in the form of a Northern hybridization or DNA chips or a sandwich ELISA or any other method as known in the art. Alternatively, the kit contains a nucleic acid substrate array comprising one or more nucleic acid sequences.

Although the present invention and its advantages have been described in detail, it should be understood that various changes, substitutions and alterations can be made herein without departing from the spirit and scope of the invention as defined in the appended claims.

Further embodiments are illustrated in the following Examples which are given for illustrative purposes only and are not intended to limit the scope of the invention.

### EXAMPLES

### Example 1—Loss of Function (LOF) and Gain of Function (GOF) Screens for Venetoclax Resistance

The B-cell lymphoma 2 (BCL-2) family includes both pro- and anti-apoptotic proteins that govern mitochondrial apoptosis. In leukemias and solid cancers, apoptosis dysregulation can result from overexpression of the anti-apoptotic BCL-2 protein that can sequester certain pro-apoptotic BH3-only proteins (BIM, BID) to avoid BAX and BAK oligomerization and subsequent mitochondrial outer membrane permeabilization. Within B cell tumors, BCL-2 dysregulation commonly arises from genetic abnormalities such as the translocation t(14;18)(q32;q21), which places BCL2 under the control of IGH promoter (in follicular lymphoma)^{1,2}; or focal deletion of chromosome 13 (del[13q14]), which leads to loss of a negative regulatory microRNA of BCL-2, miR-15a/16-1 (in chronic lymphocytic leukemia (CLL))³. Thus, BCL-2 has been a rational therapeutic target in lymphoid cancers.

Venetoclax (formerly ABT-199/GDC-0199) is a first-in-class BCL-2 inhibitor and has been recently FDA-approved for the treatment of CLL⁴. It displaces pro-apoptotic BH3-only proteins from BCL-2, allowing them to activate the mitochondrial pore-forming proteins BAK or BAX⁵. Despite its potent clinical activity in CLL cases failing control with chemotherapy regimens such as those carrying disruption of TP53⁴, disease progression on venetoclax is becoming an increasing therapeutic challenge^{6,7}.

Here, Applicants aimed to uncover the determinants of venetoclax resistance by using genome-scale survival screens, phenotypic characterization of venetoclax-resistant lymphoid cell lines, and exome-wide sequencing-based analysis of drug-resistant cell lines and primary CLL samples. The complementary analyses revealed venetoclax resistance to involve not only modulation of BCL2-family members, but also broader changes in mitochondrial metabolism.

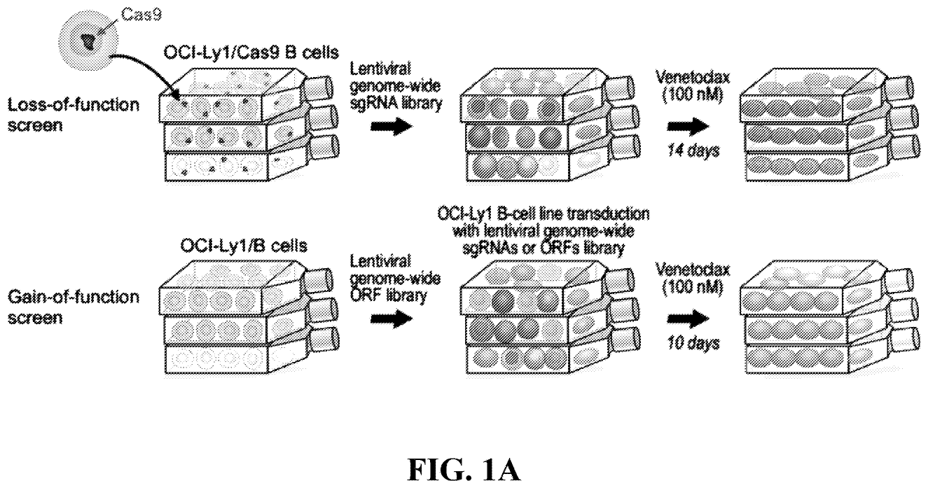

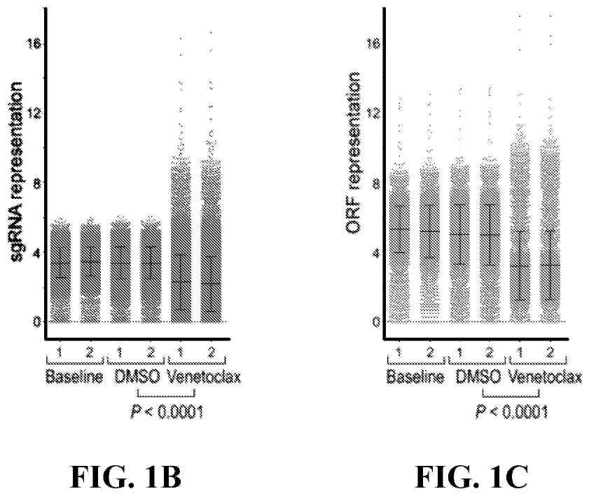

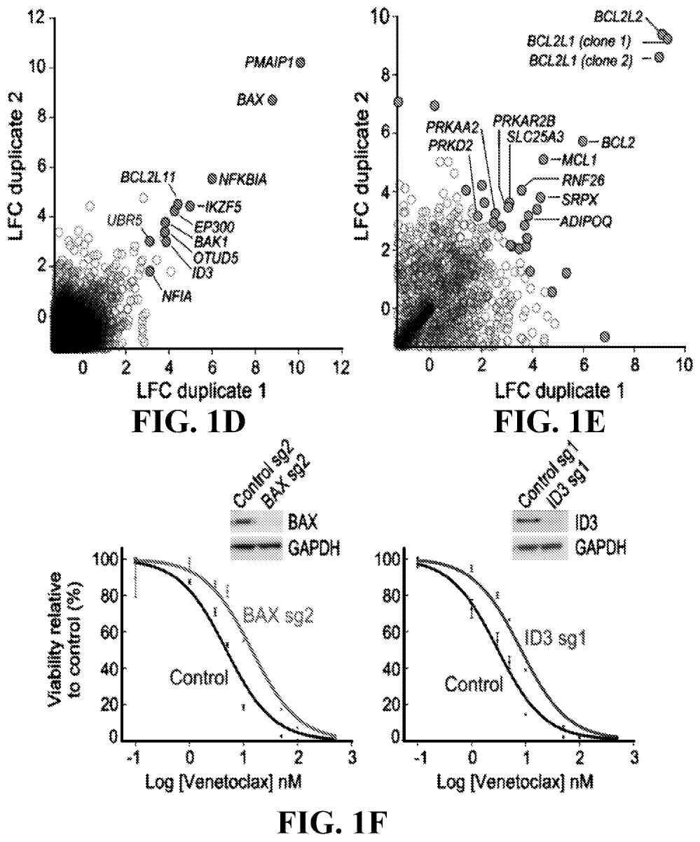

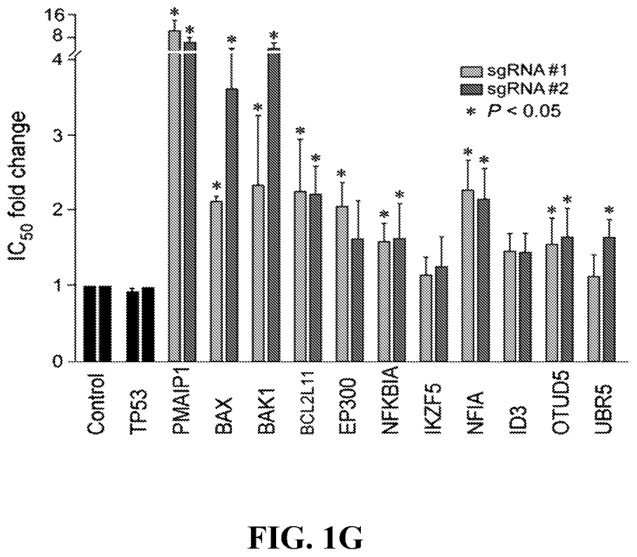

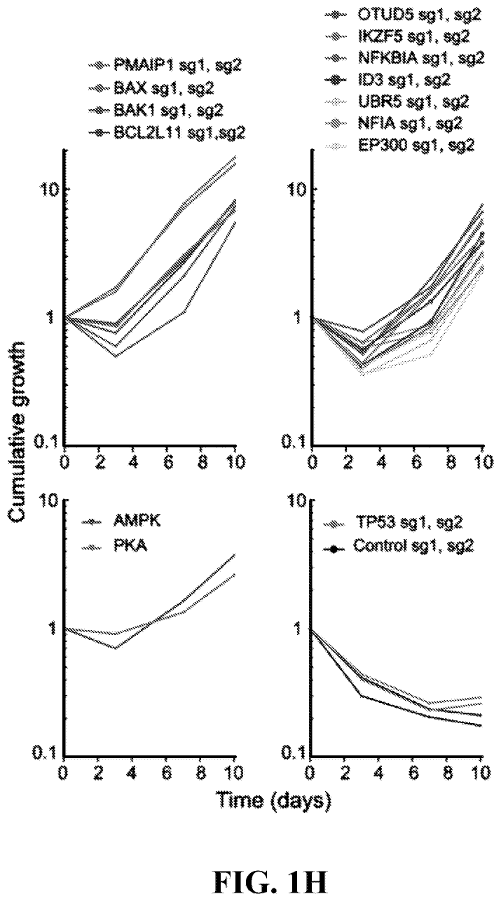

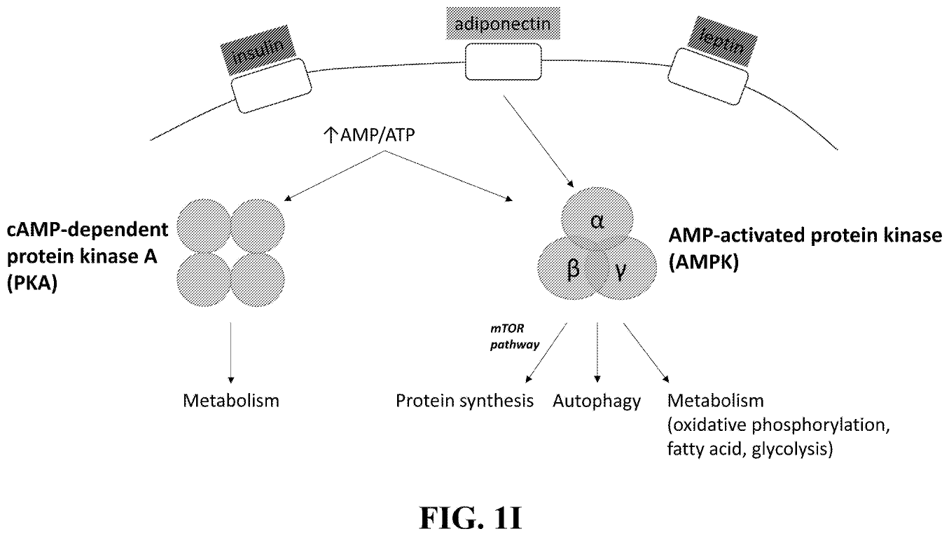

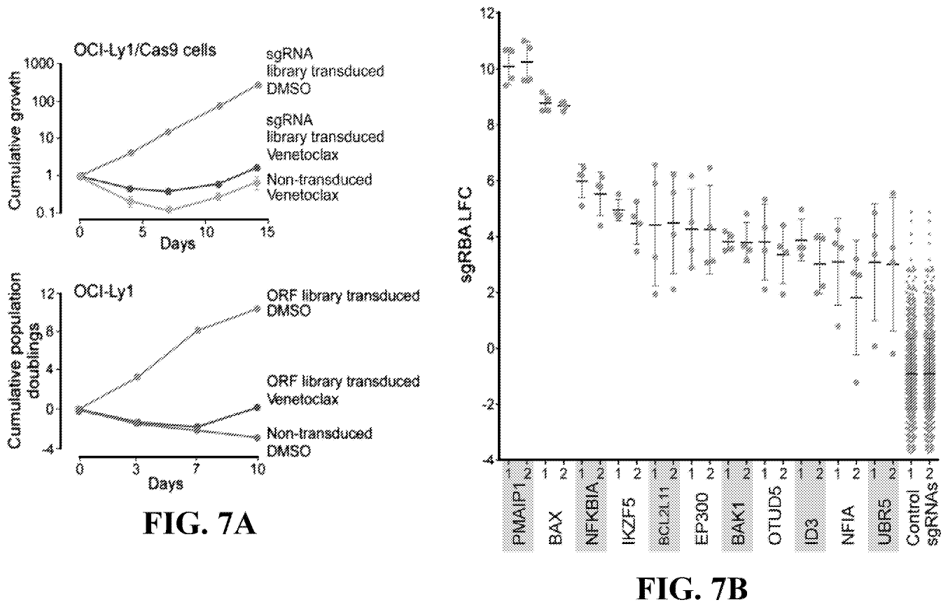

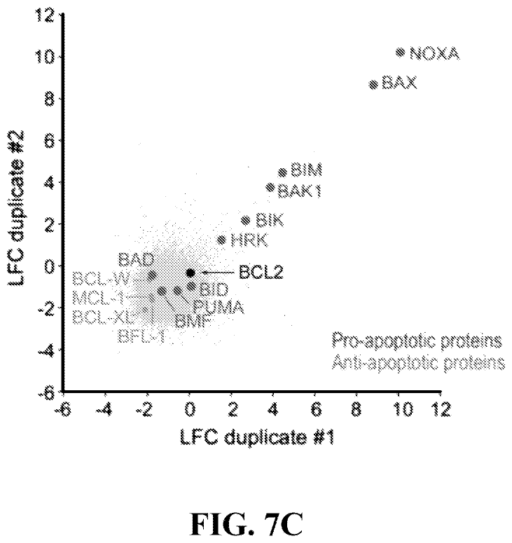

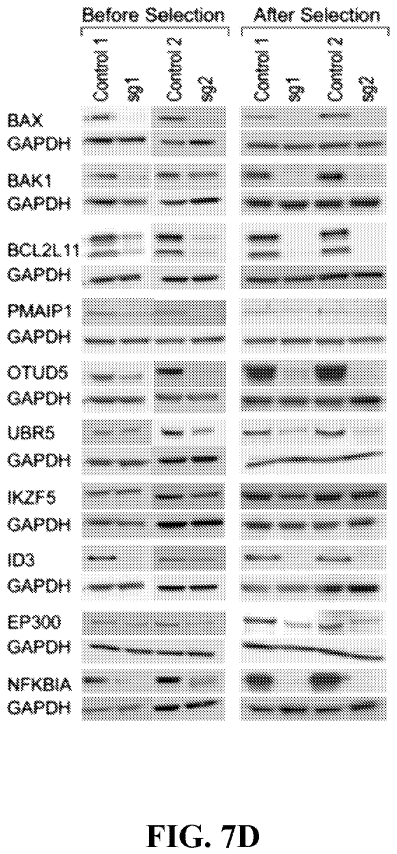

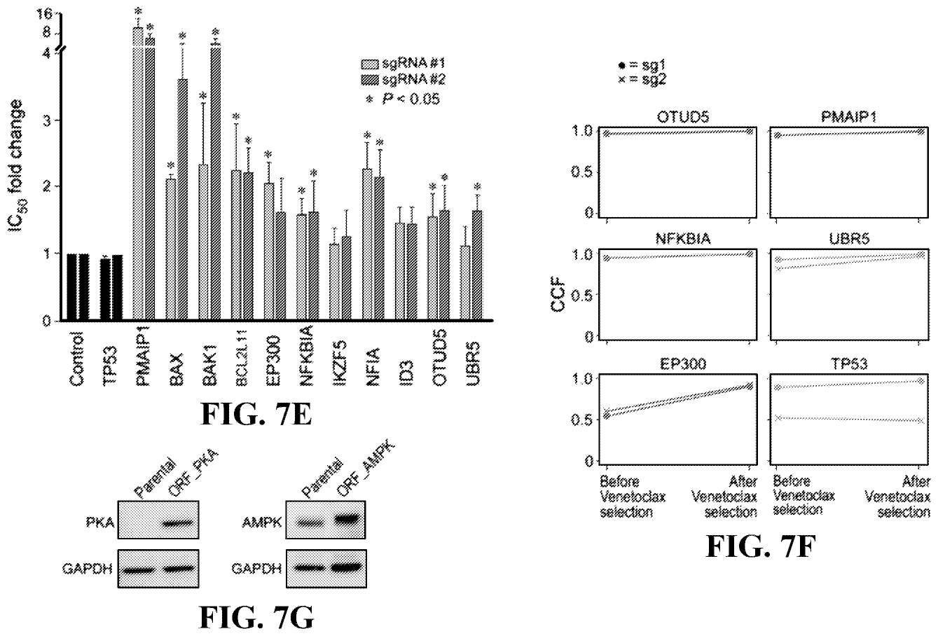

Genome-scale screens identify BCL-2 family members and novel candidate drivers of venetoclax resistance. To systematically identify drivers of resistance to venetoclax, Applicants performed parallel genome-scale loss-of-function (LOF) and gain-of-function (GOF) genetic modifier screens, using the BCL-2-driven OCI-Ly1 lymphoma cell line (FIG. 1a). For the LOF screen, OCI-Ly1 cells, modified to stably express Cas9, were infected with the Brunello lentiviral library of 76,441 sgRNAs targeting 19,114 genes and 1,000 control sgRNAs⁸, and treated with venetoclax (or DMSO, as control) for 14 days (FIG. 7a). To evaluate changes in sgRNA representation due to drug treatment, genomic DNA from viable cells on day 14 underwent sequencing for detection of the sgRNA sequences. Although average sgRNA representation was overall decreased in the transduced cells treated with venetoclax compared to duplicate sets of untreated and DMSO-treated transduced cells, a clear subset of venetoclax-treated transduced cells were enriched (FIG. 1b). By analysis with the STARS software (v1.3, Broad Institute)⁸, sgRNAs corresponding to 11 genes were consistently enriched over 2 replicates after drug treatment compared to DMSO after 14 days (STARS score >4; FIG. 1d, Table 1), with high consistency in the changes in representation amongst the 4 sgRNAs per gene (FIG. 7b). Not surprisingly, four of 11 knocked-out genes encoded pro-apoptotic BCL-2-family proteins (PMAIP1, BAX, BAK1, BCL-2L11). Moreover, the level of representation by sgRNAs corresponding to the other BCL-2 members matched their known functions as either pro- or anti-apoptotic family members (FIG. 7c). Notably, the other 7 candidate hit genes were not BCL-2-family members, but were involved in pathways highly relevant to lymphoid biology. These included NFKBIA, an inhibitor of the NF-Kappa B pathway, lymphoid transcription factors and modulators (IKZF5, ID3, EP300, NFIA), and components of the processes of ubiquitination (OTUD5, UBR5). Several of the discovered genes are recurrently mutated in B cell malignancies: UBR5 in 18% of mantle cell lymphomas⁹, ID3 in 68% of Burkitt lymphomas¹⁰, and NFKBIA in 20% of Hodgkin lymphomas¹¹.

In an analogous fashion, Applicants performed a GOF screen by using a genome-scale library including 17,255 barcoded ORFs encoding 12,952 unique proteins with at least 99% nucleotide and protein match to comprehensively identify genes that confer resistance to venetoclax when overexpressed in OCI-Ly1 cells. A total of 71 ORFs (arising from 70 genes) had a log₂ fold change (LFC) greater than 2 (FIG. 1e, Table 2). The top four genes that generated resistance when overexpressed were those encoding known anti-apoptotic proteins (BCL2L1, BCL2L2, BCL2, MCL1). Included among the top 50 genes of the screen were components of the energy-stress sensor PKA/AMPK signaling pathway (ADIPOQ, PRKAR2B, PRKAA2), mitochondrial energy metabolism (SLC25A3) or vesicle transport/autophagy (RNF26, DNM2, PRKD2, ATG5), ribosomal proteins (RPL17, RPS4Y1, RPS15A), and components of ubiquitination (OTUD6A, FBXO9, USP54) (FIG. 1e).

To confirm the LOF screen results, Applicants generated single-gene knockout OCI-Ly1 cell lines for each of the 11 hits (2 cell lines per gene, generated from the 2 most efficient sgRNAs per gene). Applicants also generated control lines corresponding to 2 non-targeting sgRNAs and for 2 sgRNAs targeting TP53 (FIG. 7d). From the GOF screen, Applicants detected two protein kinases components from related signaling pathways (PRKAR2B, PRKAA2). Applicants hence prioritized the generation of 2 OCI-Ly1 cell lines, one with overexpression of the regulatory subunit of cAMP-dependent protein kinase (protein kinase A, PKA) encoded by PRKAR2B and the other, of the catalytic subunit of the AMP-activated protein kinase (AMPK) encoded by PRKAA2, both of which are key regulators of cellular metabolism (FIG. 7e)^{12,13}.

Across the individual knockout cell lines, Applicants observed a decreased sensitivity to venetoclax with median increase in IC50 of 2.1-fold (range, 1.3- to 13.8-fold; P<0.05, extra-sum-of-squares F test) compared to cell lines transduced with 2 non-targeting control sgRNAs (FIG. 1f, FIG. 7f). Moreover, all generated knockout and overexpression lines showed increased cumulative growth over 10 days of in vitro venetoclax treatment (FIG. 1h). Exposure of the knockout cell lines to venetoclax increased the degree of depletion of the targeted protein and enriched the frequency of frameshift indels generated by the introduction of sgRNAs into the cell lines (FIG. 7d, g, Table 3). Altogether, these results confirmed the on-target effects of the sgRNAs and the ORFs identified in the analyses of the parallel genome-wide screens.

### Determining the Signature of Venetoclax Resistance

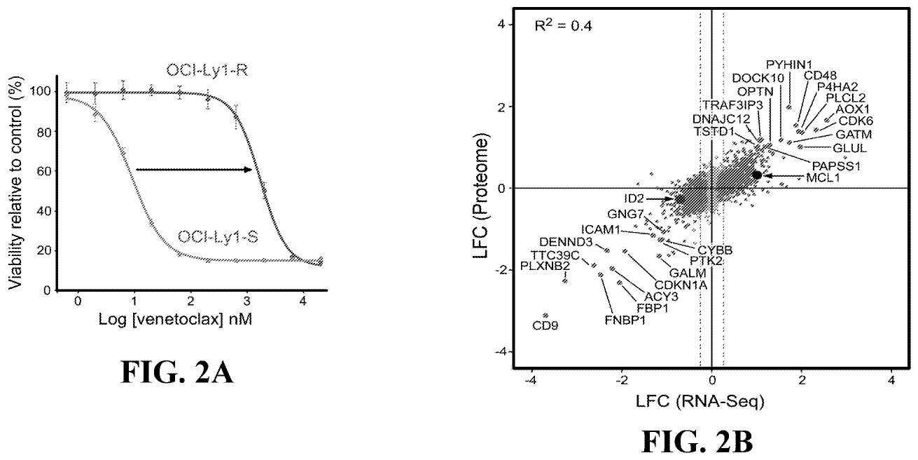

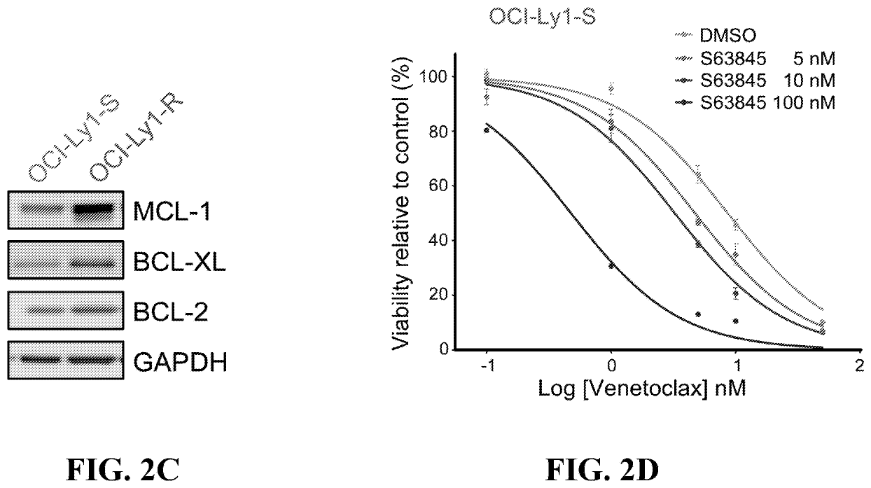

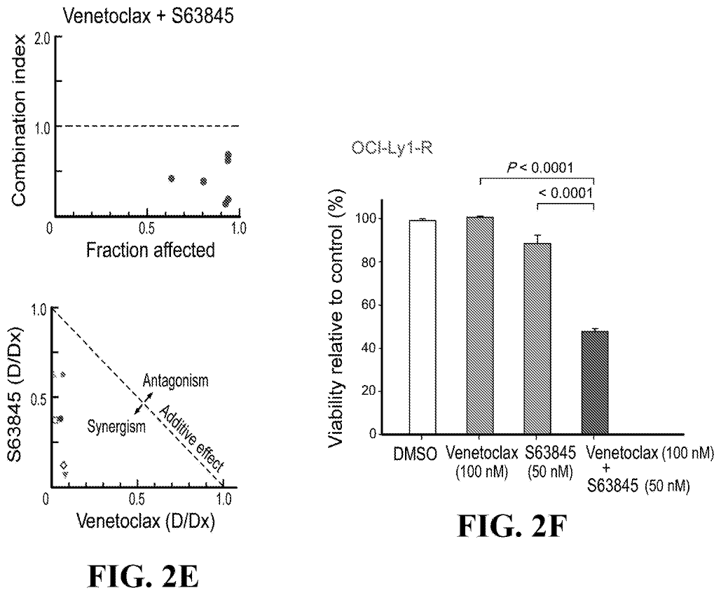

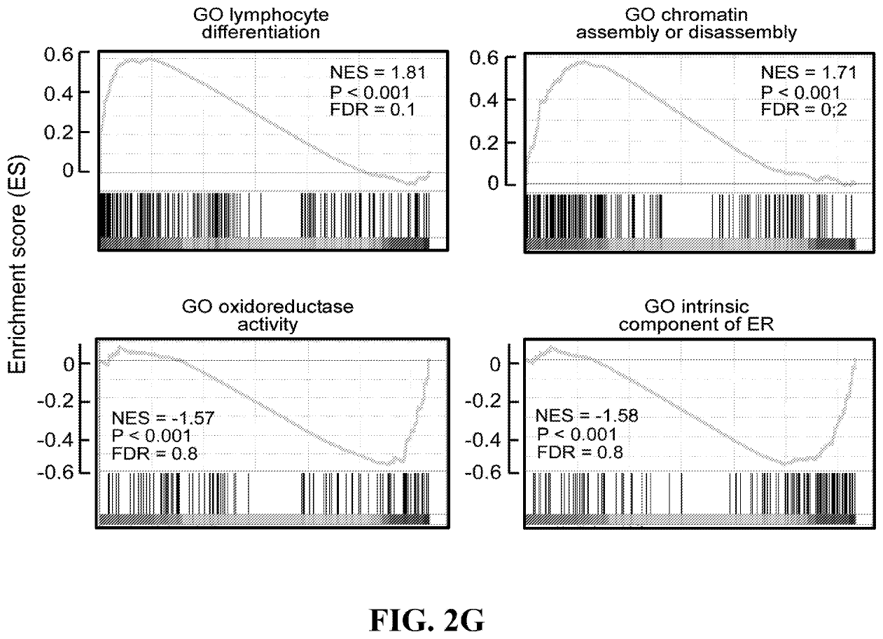

In complementary experiments, Applicants characterized the transcript-level and protein expression profiles of a venetoclax-resistant cell line (OCI-Ly1-R, IC50 1 μM), generated through chronic in vitro drug exposure of the parental (OCI-Ly1-S, IC50 4 nM) cells (FIG. 2a). By RNA-sequencing (RNA-Seq) of the resistant and sensitive cell lines, Applicants identified 19 upregulated and 28 downregulated genes (adjusted P-value <0.05; |LFC|>2) (FIG. 8a, Table 3). Comparison of the transcriptomes and the proteomes (identified via mass spectrometry-based proteomics, see Methods) of the OCI-Ly1-S and OCI-Ly1-R cells revealed close concordance across these two platforms (R²=0.4, all genes; R²=0.8, restricted to differentially expressed genes; FIG. 2b), with 14 upregulated and 13 downregulated proteins (adjusted P-value <0.05, |LFC|>2) (Tables 3 and 4).

When evaluated at the gene-level, MCL-1 emerged as the only significantly and coordinately deregulated transcript and protein that also overlapped with the gene hits from the genome-scale screens (FIG. 2b). MCL-1 overexpression has been previously reported in the characterizations of cancer cell lines rendered resistant to BCL-2 inhibition and has been described to sequester the pro-apoptotic BIM protein^{14,15}. Applicants confirmed the relative increase in protein expression of MCL-1 in OCI-Ly1-R cells compared to OCI-Ly1-S cells (FIG. 2c), and observed in vitro synergy between venetoclax and the MCL-1 inhibitor S63845¹⁶ on OCI-Ly1-S cells (combination index <1, FIG. 2d-e). MCL-1 inhibition could furthermore restore venetoclax sensitivity to the OCI-Ly1-R cells (FIG. 2f). These results confirm a key role of MCL-1 overexpression in mediating venetoclax resistance.

In contrast to gene-level analysis, pathway-level geneset enrichment analysis (GSEA) based on RNAseq data revealed 35 significantly enriched pathways (nominal P-value <0.05, FDR <0.25) (Table 5). Consistent with pathway-level results from Applicants' gain- and loss-of-function screens, positively regulated pathways included lymphoid differentiation and chromatin maintenance, while top negatively regulated pathways related to metabolism and the endoplasmic reticulum (nominal P-value <0.002, FDR <0.9) (FIG. 2g). In addition, as observed in Applicants' functional genomics screens, the most coordinately upregulated transcripts and proteins (Tables 3 and 4) originated from genes critical to cellular metabolism (AOX1, GLUL, PAPSS1, GATM, TSTD1, GALM, FBP1) (FIG. 2b). GLUL encodes the glutamine synthetase that plays a role in cell survival¹⁷; FBP1 encodes the fructose-bisphosphatase 1 and its repression was previously shown to efficiently promote glycolysis¹⁸. The other upregulated transcripts/proteins highlighted other mechanisms of potential interest, including cell cycle regulation (CDK6, CDKN1A [encoding p21], TT39C), B-cell biology (DOCK10) as well as autophagy (DENND3, OPTN) and reactive oxygen species generation (CYBB).

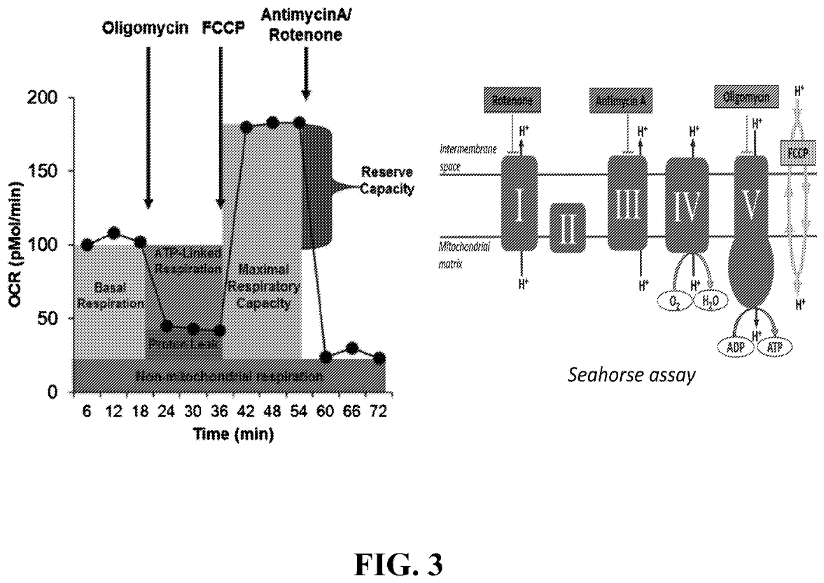

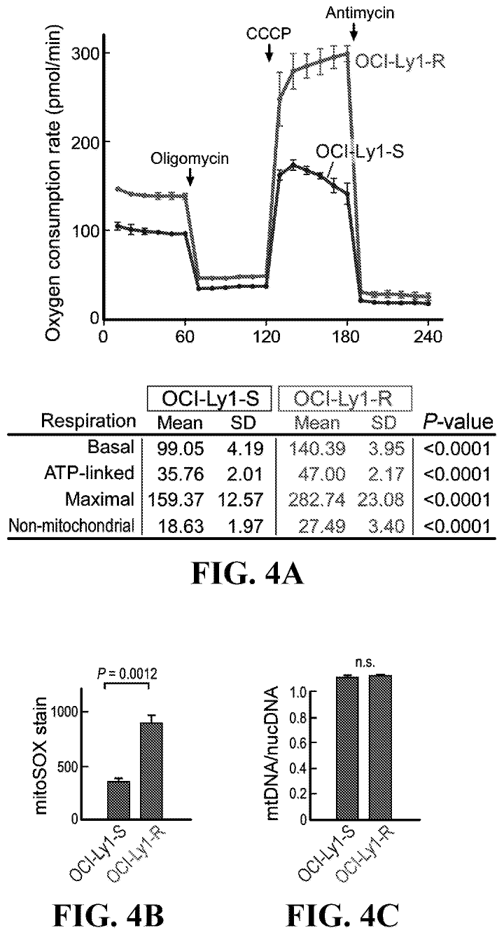

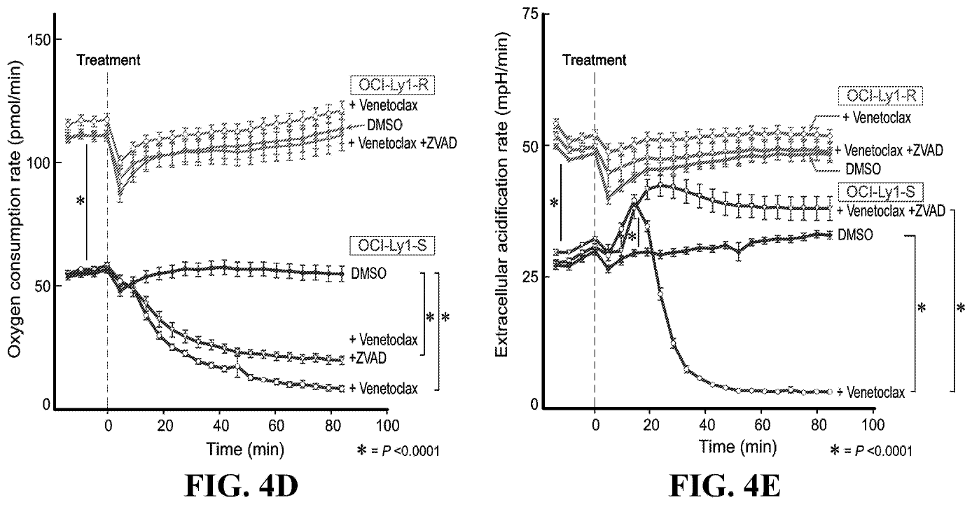

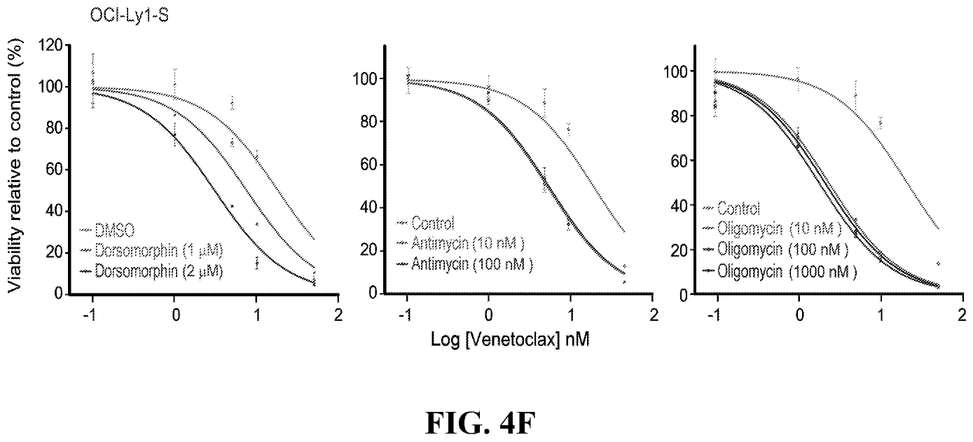

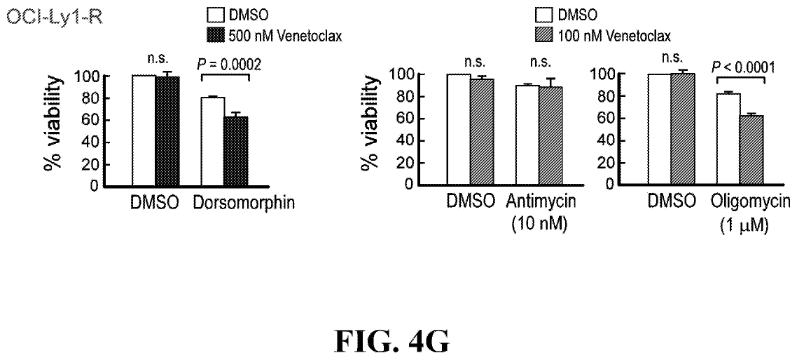

Metabolic reprogramming plays a critical role in the resistance to BCL-2 inhibition. Given the dysregulation of proteins critical to AMPK signaling and metabolism in both the GOF screen and in OCI-Ly1-R cells, Applicants hypothesized that metabolic reprogramming also contributes to resistance of malignant B cells to venetoclax. A recent genome-wide CRISPR screen identified AMPK subunits as regulators of oxidative phosphorylation¹⁹. Applicants therefore evaluated mitochondrial respiration by measuring the oxygen consumption rate over time following the addition of mitochondrial electron transplant chain (mETC) modulators (Seahorse assay, Methods). Compared to OCI-Ly1-S cells, OCI-Ly1-R cells demonstrated markedly higher rate of oligomycin sensitive oxygen consumption, suggesting a state of higher oxidative phosphorylation (OXPHOS) (FIG. 3a, P<0.0001). Applicants also noted the OCI-Ly1-R cells to have higher levels of reactive oxygen species and higher mitochondrial membrane potential (FIG. 3b, FIG. 8b). Applicants ascertained that this was not a result of an increased mass of mitochondria per cell in the resistant cells, since the quantity of mitochondrial DNA was equivalent between the drug-resistant and -sensitive cells (FIG. 3c). Applicants found that OCI-Ly1-R also exhibited a higher basal level of glycolysis, as assessed by extracellular acidification rate (ECAR) (FIG. 3e, P<0.0001).

To investigate whether venetoclax directly affects cellular energy metabolism, Applicants measured oxygen consumption of the cell lines following venetoclax exposure. Consistent with impairment of OXPHOS by venetoclax, Applicants observed both an immediate decrease in oxygen consumption (by 2-fold within 20 minutes after drug exposure) and an immediate increase in ECAR following venetoclax in the OCI-Ly1-S cells, but not in the OCI-Ly1-R cells (FIGS. 3d and 3e, P<0.0001). The impact on OXPHOS was apoptosis-independent, as it was not fully prevented with the caspase inhibitor zVAD.

In line with these findings, the AMPK inhibitor dorsomorphin (compound C) and drugs targeting the mETC (i.e. oligomycin, antimycin) synergized with venetoclax in the OCI-Ly1-S cells (combination index <1, FIG. 3f, FIG. 8c). Additionally, dorsomorphin and oligomycin could each restore sensitivity to venetoclax in the OCI-Ly1-R cells (FIG. 3g). Altogether, these results show that venetoclax not only induces cell death through caspase activation but also induces rapid changes in metabolism. Furthermore, multiple lines of evidence suggest that resistance to this compound results at least in part from metabolic reprogramming.

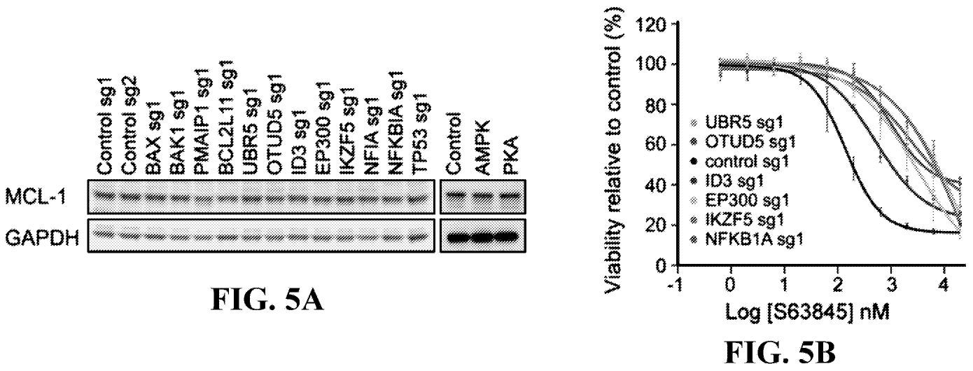

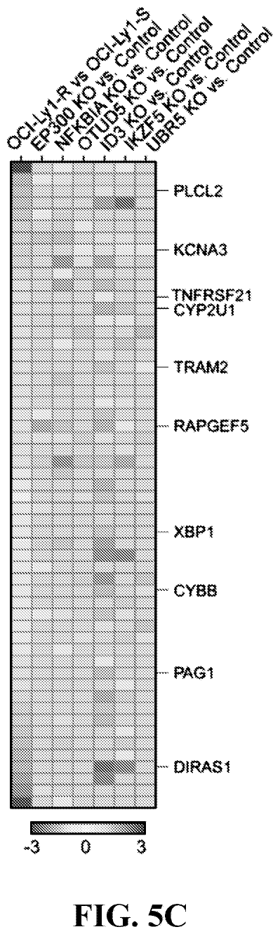

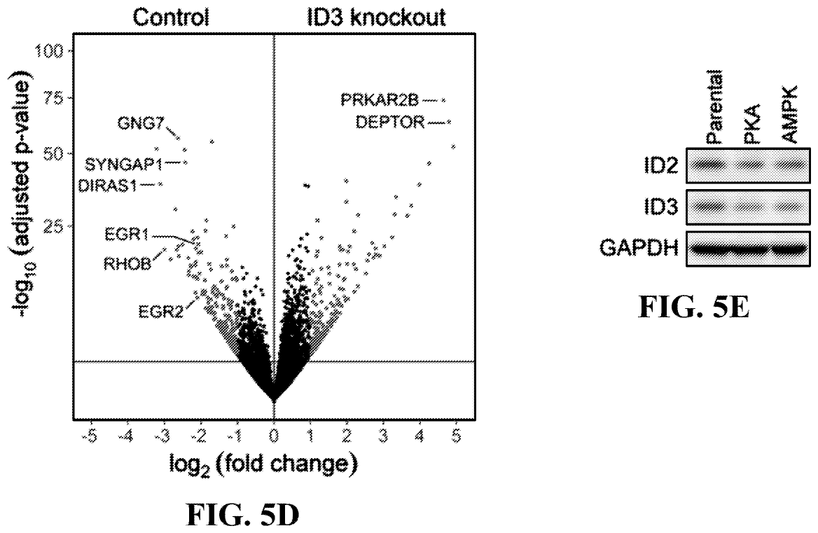

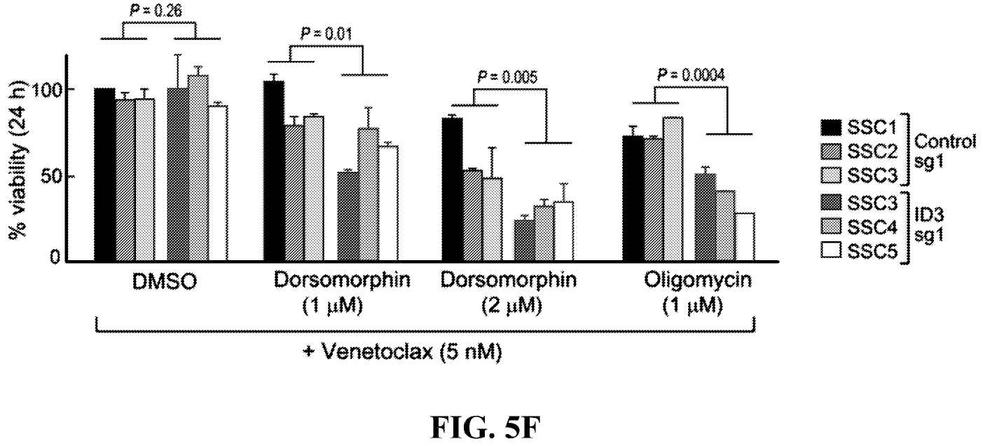

To define the possible mechanisms by which the candidate hit genes identified from the survival screens were related to venetoclax resistance, Applicants first evaluated MCL-1 expression in the respective knockout and overexpression cell lines. None of 14 tested cell lines demonstrated elevated MCL-1 levels (FIG. 5a) nor clear sensitivity to MCL-1 inhibition (FIG. 5b), suggesting their engagement in alternative mechanisms of resistance to venetoclax. Transcriptome profiling of each of the knockout cell lines corresponding to 6 non-BCL-2 family member gene candidates revealed the knockout line of the lymphoid transcription factor ID3 as the most similar to the OCI-Ly1-R cell line (FIG. 5c). Shared were transcripts involved in lymphoid signaling (PLCL2, KCNA3, PAG1) or transcription (XBP1), metabolism (CYP2U1, CYBB), apoptosis (TNFRSF21) and Ras signaling (DIRAS1, GNG7, RAPGEF5). Of note, ID2 (closely related to ID3) was amongst the coordinately deregulated transcripts and proteins in the OCI-Ly1-R cell line (indicated in FIG. 2b). Recent work has implicated lymphoid transcriptional factors as metabolic gatekeepers²⁰ and the ID family of genes has been previously suggested to regulate the function of specific mETC complexes, thereby modulating mitochondrial OXPHOS^{21,22}.

In keeping with these reports, PRKAR2B, which Applicants had previously uncovered in the GOF screen, was the most significantly upregulated gene of the ID3 knockout cell line (adjusted P-value <0.05, LFC>2; FIG. 5d). Other strongly dysregulated transcripts fell in the mTOR pathway (e.g. DEPTOR [DEP domain-containing mTOR-interacting protein] gene), and the pathways of Ras signaling (DIRAS1, RHOB, GNG7, SYNGAP1 genes) and B-cell differentiation (EGR1, EGR2). Conversely, overexpression of PRKAR2B (encoding a PKA subunit) or PRKAA2 (encoding the catalytic AMPK subunit) led to repression of both ID3 and ID2 genes (FIG. 5e). Single cell clones derived from the ID3 knockout cell line (FIG. 8d) were more sensitive to the combination of venetoclax and either dorsmorphin or oligomycin than control cells (FIG. 5f), suggesting AMPK dependency and metabolic adaptation related to ID3 repression. PKA and AMPK overexpressing cells demonstrated similar increased sensitivity to the combination of venetoclax and oligomycin (FIG. 8e). Thus, metabolic dysregulation through ID3 links the findings of Applicants' complementary GOF and LOF screens.

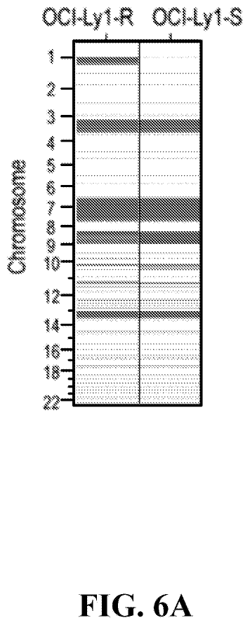

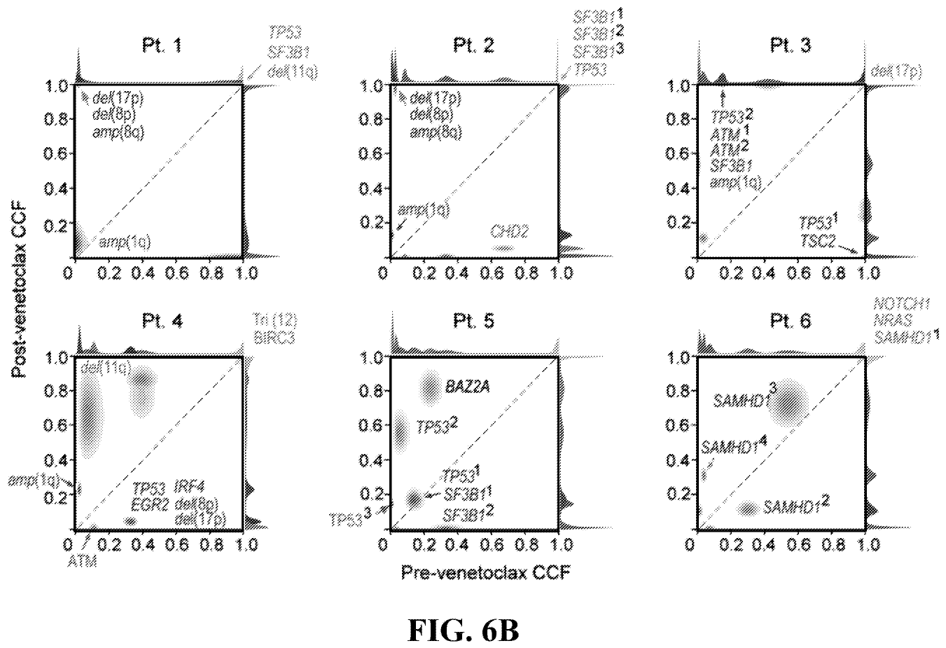

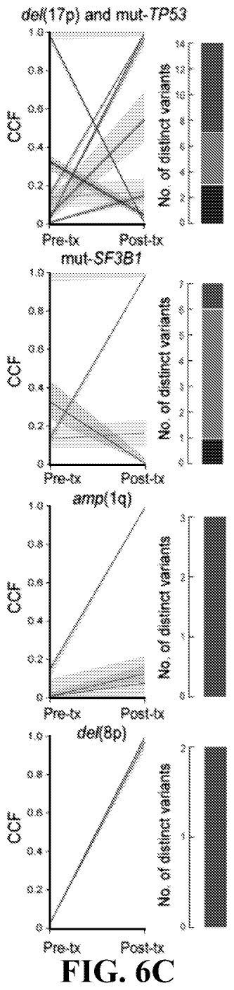

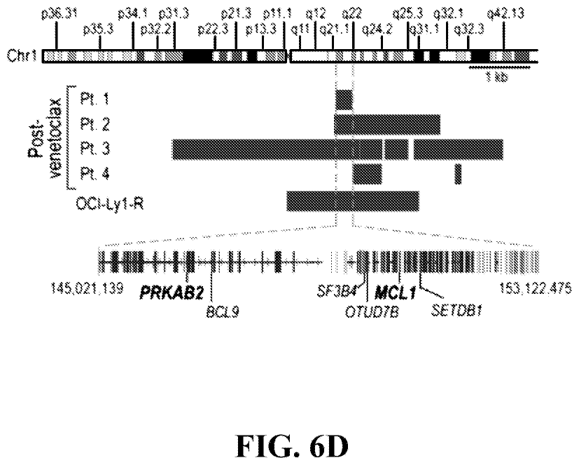

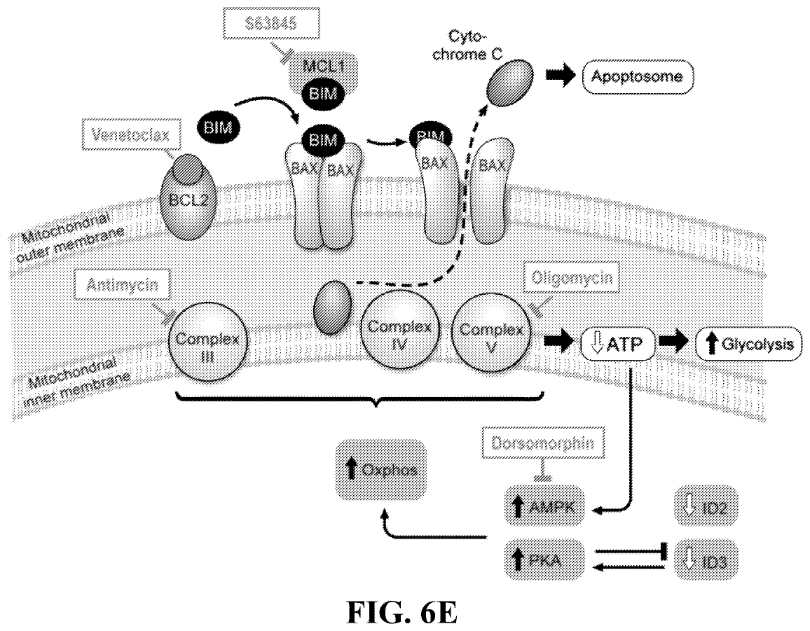

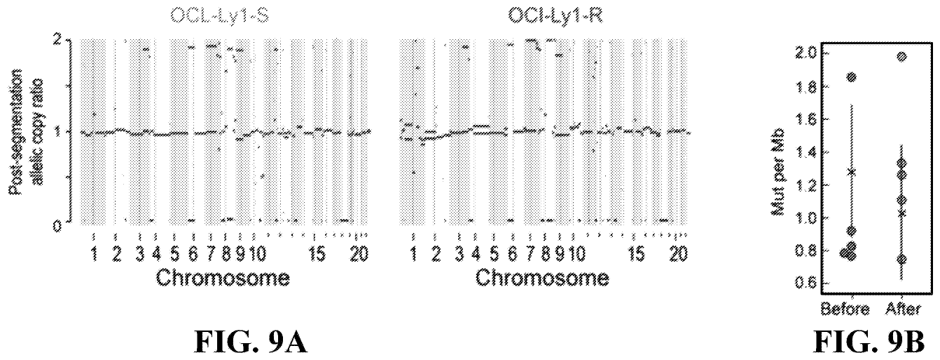

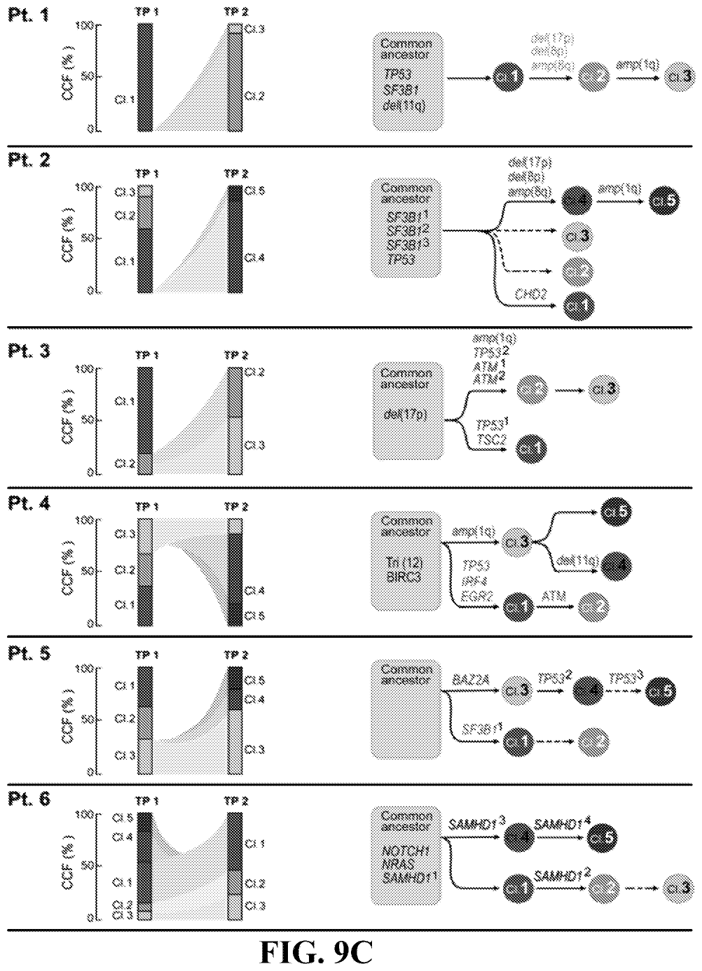

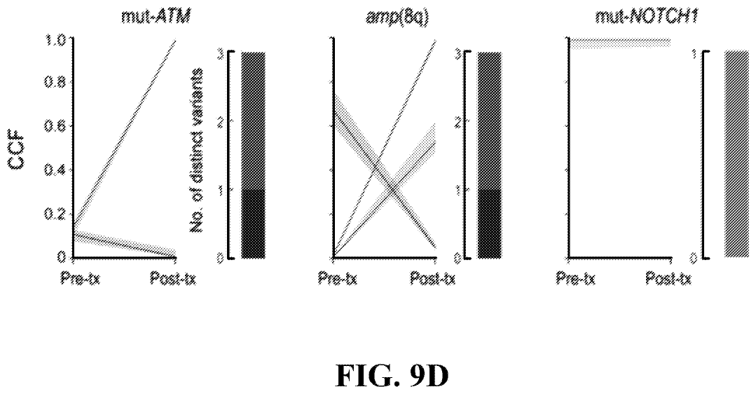

Venetoclax resistance in CLL patients is associated with clonal shifts. To determine if there was a genetic basis for the observed drug resistance in the OCI-Ly1-R cells, Applicants compared the results of whole-exome sequencing (WES) of DNA isolated from the OCI-Ly1-R and OCI-Ly1-S cell lines. No non-silent somatic single nucleotide variants (sSNVs) overlapping with Applicants' screen data were identified except in BCL2, a common target of aberrant somatic hypermutation frequently affected in B lymphomas and most B-cell lines.²¹ However, none involved BH3 domain residues previously linked to venetoclax resistance in cell lines²⁴. Regarding somatic copy number variations (sCNVs), a clear region was amplified on chromosome 1q23 (amp[1q]) in the OC-Ly1-R but not in the OCI-Ly-S cells. No other differential sCNVs were identified (FIG. 6a, FIG. 9a).

Similarly, in a WES-based analysis of paired CLL DNA samples isolated from 6 patients at treatment initiation and at the time of progression on venetoclax (median time to relapse 16.4 months [range, 5.1-22.8].

TABLE 6
Venetoclax resistance in CLL patients.

| | | | Pre-venetoclax | | | | |
|---|---|---|---|---|---|---|---|
| | | | baseline | | | | Time to |
| | Age (y) | | cytogenetic | IGHV | Daily | Best | progression |
| Patient | Gender | Prior therapy | features | status | dose | response | (months) |
| 1 | 70 F | FCR | del(17p), | ND | 400 mg | PR | 22.8 |
| | | | del(11q), CK | | | | |
| 2 | 80 M | FCR, BR, | del(17p), | UM | 400 mg | PR | 7.1 |
| | | R-MP, | tri 12, CK | | | | |
| | | alemtuzumab | | | | | |
| 3 | 64 F | FCR | del(17p) | UM | 400 mg | PR | 16.4 |
| 4 | 54 F | FCR, R-MP | del(17p), | ND | 400 mg | PR | 5.1 |
| | | | del(11q), tri 12, | | | | |
| | | | del(13q), CK | | | | |
| 5 | 66 M | FR, BR, | normal FISH | UM | 400 mg | PR | 8.2 |
| | | ibrutinib, | | | | | |
| | | idelalisib | | | | | |
| 6 | 46 M | FCR, BTKi | normal FISH | UM | 1200 | PR | 22.8 |
| | | (AVL292) | | | mg then | | |
| | | | | | 400 mg | | |

TABLE 7
Sample Characteristics

| | Pre-venetoclax | Post-venetoclax | Germline |
|---|---|---|---|
| Patient | sample | sample | sample |
| 1 | Marrow MCs | Marrow MCs | saliva |
| 2 | PBMCs | PBMCs | saliva |
| 3 | FFPE marrow | FFPE marrow | absent |
| | biopsy | biopsy | |
| 4 | FFPE marrow | Fresh lymph | saliva |
| | biopsy | node biopsy | |
| 5 | PBMCs | PBMCs | saliva |
| 6 | PBMCs | PBMCs | Saliva |

Mononuclear cells were isolated with Ficoll/Hypaque density-gradient centrifugation, cryopreserved with 10% DMSO and stored in vapor-phase liquid nitrogen until the time of analysis. MCs, mononuclear cells; PB, peripheral blood; FFPE, formalin-fixed, paraffin-embedded.

Applicants did not identify any sSNVs in BCL2 itself at baseline or at progression, nor in any coding region of BCL-2 family members, despite the observation of marked clonal shifts in all patients (FIG. 6b-c, Methods). No differences in the median mutation rate between baseline and relapse samples across patients were observed (FIG. 9b; Mann-Whitney two-tailed P=0.40). Numerous instances of shifting cancer cell fractions (CCF) of subclones with ATM, TP53 or SF3B1 mutation, even of distinct subclones with differing mutated alleles of the same gene within an individual (FIG. 6c, FIG. 9c-d) were observed, but no sSNVs of known CLL driver mutations were consistently selected with resistance.

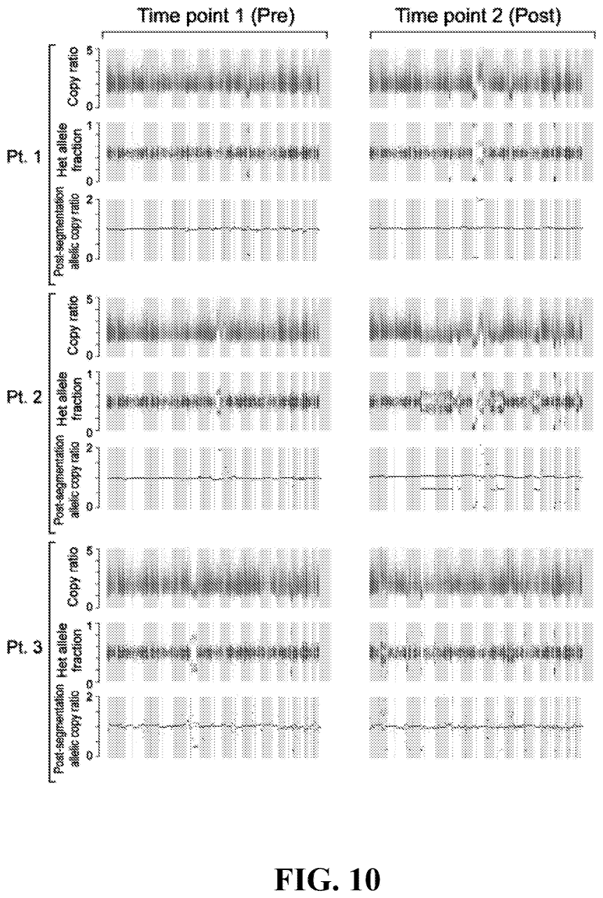

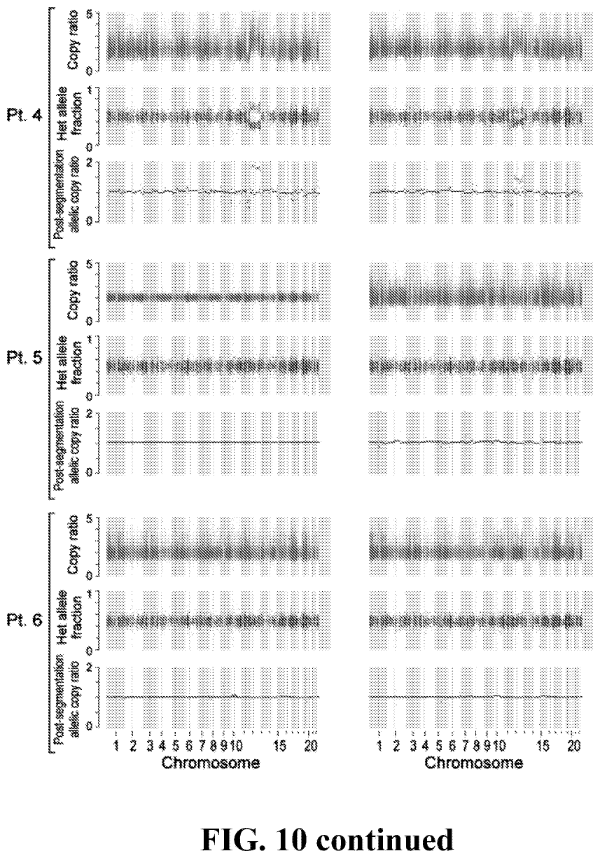

The shifts in sCNVs were more revealing. In both Patients 1 and 2, resistance was associated with a large expansion of a subclone carrying a combination of recurrent cancer alterations comprised of del(17p) [TP53], amp(8q) [MYC] and del(8p) [encompasses the TRAIL receptor] (FIG. 5b, FIGS. 9b and 10). Applicants previously described enrichment of del(8p) in the setting of resistance to the targeted BTK inhibitor ibrutinib²⁵, and BCL-2 inhibition has been shown to sensitize human cancer cells to TRAIL-induced apoptosis by inducing the expression of its receptor²⁶. Applicants' finding of amp(8q) (MYC locus) together with the recently reported del(9p) (CDKN2A/B locus) in venetoclax resistant patients²⁷, supports the idea that changes in cell cycle regulation are important in resistance. Applicants noted that Patients 1 and 2 both had truncal SF3B1 and TP53 mutations, suggesting the selection of this complex subclone as a common trajectory (FIG. 6b, FIG. 9c). In line with the cell line data, Applicants observed growing subclones with amp(1q) in 4 of 6 cases (Patients 1, 2, 3 and 4; FIG. 6b, FIG. 9d and FIG. 10). Applicants confirmed a common region of gain of 8 Mb when overlapping cell lines and Patients 1, 2, and 3 data. Contained within this amplified region were several genes of interest including MCL1 and PRKAB2, encoding the regulatory subunit of AMPK (FIG. 6d). In line with cell line data, these results support the involvement of MCL-1 and AMPK in patients.

TABLE 8
Patient sSNVs and sCNVs

| Hugo_Symbol | Patient ID | Chromosome | Start_position | End_position | Variant_Classification |
|---|---|---|---|---|---|
| Unknown | MDA-1 | 20 | 5049934 | 5049934 | IGR |
| AATK | MDA-1 | 17 | 79100307 | 79100307 | Silent |
| ABCD4 | MDA-1 | 14 | 74769649 | 74769649 | 5′UTR |
| ABCF1 | MDA-1 | 6 | 30557939 | 30557939 | Silent |
| ABTB2 | MDA-1 | 11 | 34189512 | 34189512 | Missense_Mutation |
| ACAD10 | MDA-1 | 12 | 112140025 | 112140025 | Missense_Mutation |
| ACAT1 | MDA-1 | 11 | 108013201 | 108013201 | Silent |
| ACD | MDA-1 | 16 | 67693054 | 67693054 | Intron |
| ACIN1 | MDA-1 | 14 | 23547434 | 23547434 | Silent |
| ACKR3 | MDA-1 | 2 | 237489447 | 237489447 | Silent |
| ADC | MDA-1 | 1 | 33563771 | 33563771 | Missense_Mutation |
| ADORA3 | MDA-1 | 1 | 112031338 | 112031338 | Missense_Mutation |
| AHNAK2 | MDA-1 | 14 | 105418236 | 105418236 | Intron |
| ALG8 | MDA-1 | 11 | 77850647 | 77850647 | 5′UTR |
| ANO9 | MDA-1 | 11 | 418786 | 418786 | Silent |
| ARMC12 | MDA-1 | 6 | 35716408 | 35716408 | Missense_Mutation |
| ARSK | MDA-1 | 5 | 94901826 | 94901826 | Missense_Mutation |
| ASAP2 | MDA-1 | 2 | 9458703 | 9458703 | Silent |
| ATP13A1 | MDA-1 | 19 | 19766380 | 19766380 | Silent |
| ATP6V0B | MDA-1 | 1 | 44443534 | 44443534 | Intron |
| ATP6V0B | MDA-1 | 1 | 44443854 | 44443854 | 3′UTR |
| ATP8B4 | MDA-1 | 15 | 50223345 | 50223345 | Missense_Mutation |
| BFSP1 | MDA-1 | 20 | 17474966 | 17474966 | Missense_Mutation |
| C15orf27 | MDA-1 | 15 | 76484383 | 76484383 | Silent |
| C15orf39 | MDA-1 | 15 | 75500385 | 75500385 | Missense_Mutation |
| C16orf45 | MDA-1 | 16 | 15677033 | 15677033 | Intron |
| C19orf18 | MDA-1 | 19 | 58469962 | 58469962 | 3′UTR |
| C21orf58 | MDA-1 | 21 | 47737231 | 47737231 | 5′UTR |
| C2CD2 | MDA-1 | 21 | 43327136 | 43327136 | Missense_Mutation |
| CACNA1S | MDA-1 | 1 | 201008831 | 201008831 | 3′UTR |
| CAD | MDA-1 | 2 | 27438370 | 27438370 | 5′Flank |
| CADM3 | MDA-1 | 1 | 159169596 | 159169596 | Silent |
| CAPRIN2 | MDA-1 | 12 | 30866733 | 30866733 | Missense_Mutation |
| CASP5 | MDA-1 | 11 | 104879640 | 104879640 | Intron |
| CCDC57 | MDA-1 | 17 | 80146161 | 80146161 | Missense_Mutation |
| CDC42BPB | MDA-1 | 14 | 103412888 | 103412888 | Missense_Mutation |
| CDK6 | MDA-1 | 7 | 92462649 | 92462649 | 5′UTR |
| CENPT | MDA-1 | 16 | 67859170 | 67859170 | IGR |
| CEPT1 | MDA-1 | 1 | 111724875 | 111724875 | Missense_Mutation |
| CES4A | MDA-1 | 16 | 67034915 | 67034915 | Intron |
| CFTR | MDA-1 | 7 | 117171082 | 117171082 | Missense_Mutation |
| CHRNA4 | MDA-1 | 20 | 61975048 | 61975048 | IGR |
| CHRNA4 | MDA-1 | 20 | 61977100 | 61977100 | 3′UTR |
| CHRNB3 | MDA-1 | 8 | 42587276 | 42587276 | Missense_Mutation |
| CHTF18 | MDA-1 | 16 | 839639 | 839639 | Missense_Mutation |
| CLTB | MDA-1 | 5 | 175843297 | 175843297 | Missense_Mutation |
| COL18A1 | MDA-1 | 21 | 46925155 | 46925155 | Silent |
| COMMD4 | MDA-1 | 15 | 75632451 | 75632451 | 3′UTR |
| CSMD2 | MDA-1 | 1 | 34192274 | 34192274 | Missense_Mutation |
| CTBP1 | MDA-1 | 4 | 1222131 | 1222131 | Splice_Site |
| CYP1A2 | MDA-1 | 15 | 75047364 | 75047364 | Missense_Mutation |
| DEFA4 | MDA-1 | 8 | 6793444 | 6793444 | 3′UTR |
| DEFB1 | MDA-1 | 8 | 6728155 | 6728155 | 3′UTR |
| DIABLO | MDA-1 | 12 | 122710543 | 122710543 | Missense_Mutation |
| DOPEY2 | MDA-1 | 21 | 37581028 | 37581028 | Silent |
| DST | MDA-1 | 6 | 56472185 | 56472185 | Missense_Mutation |
| DYNC1H1 | MDA-1 | 14 | 102493523 | 102493523 | Silent |
| DYSF | MDA-1 | 2 | 71778274 | 71778274 | Silent |
| EIF2AK4 | MDA-1 | 15 | 40284365 | 40284365 | Intron |
| ENPEP | MDA-1 | 4 | 111469387 | 111469387 | Missense_Mutation |
| EP300 | MDA-1 | 22 | 41575101 | 41575101 | 3′UTR |
| EPHB4 | MDA-1 | 7 | 100410369 | 100410369 | Splice_Site |
| ERCC6 | MDA-1 | 10 | 50732137 | 50732137 | Missense_Mutation |
| ESYT1 | MDA-1 | 12 | 56532010 | 56532010 | Missense_Mutation |
| EXPH5 | MDA-1 | 11 | 108384034 | 108384034 | Missense_Mutation |
| EXPH5 | MDA-1 | 11 | 108381304 | 108381304 | Missense_Mutation |
| FAM111B | MDA-1 | 11 | 58892455 | 58892455 | Silent |
| FAM65B | MDA-1 | 6 | 24936164 | 24936164 | 5′UTR |
| FAM71B | MDA-1 | 5 | 156593004 | 156593004 | Missense_Mutation |
| FBXL16 | MDA-1 | 16 | 745861 | 745861 | Silent |
| FLNB | MDA-1 | 3 | 58109215 | 58109215 | Silent |
| FLYWCH2 | MDA-1 | 16 | 2946755 | 2946755 | Missense_Mutation |
| FNBP4 | MDA-1 | 11 | 47788853 | 47788853 | 5′UTR |
| FNBP4 | MDA-1 | 11 | 47776121 | 47776121 | Missense_Mutation |
| FZD8 | MDA-1 | 10 | 35928269 | 35928269 | 3′UTR |
| GCC1 | MDA-1 | 7 | 127223259 | 127223259 | Silent |
| GNA15 | MDA-1 | 19 | 3148650 | 3148650 | Silent |
| GPR144 | MDA-1 | 9 | 127230406 | 127230406 | Silent |
| GRIP1 | MDA-1 | 12 | 66742963 | 66742963 | Missense_Mutation |
| HAVCR1 | MDA-1 | 5 | 156482307 | 156482307 | Missense_Mutation |
| HGD | MDA-1 | 3 | 120393783 | 120393783 | Silent |
| HNRNPM | MDA-1 | 19 | 8528518 | 8528518 | Missense_Mutation |
| HNRNPUL2 | MDA-1 | 11 | 62489302 | 62489302 | Missense_Mutation |
| HSD17B12 | MDA-1 | 11 | 43876811 | 43876811 | 3′UTR |
| HSDL2 | MDA-1 | 9 | 115232876 | 115232876 | 3′UTR |
| IGHM | MDA-1 | 14 | 106322331 | 106322331 | RNA |
| INTS6 | MDA-1 | 13 | 51938412 | 51938412 | IGR |
| IQGAP1 | MDA-1 | 15 | 90931555 | 90931555 | 5′UTR |
| ITPR1 | MDA-1 | 3 | 4687151 | 4687151 | Silent |
| JMJD1C | MDA-1 | 10 | 64927838 | 64927838 | Silent |
| JMJD1C | MDA-1 | 10 | 64974886 | 64974886 | Missense_Mutation |
| JPH3 | MDA-1 | 16 | 87678135 | 87678135 | Silent |
| JPH4 | MDA-1 | 14 | 24040505 | 24040505 | Missense_Mutation |
| KDELR2 | MDA-1 | 7 | 6505949 | 6505949 | Silent |
| KIAA0020 | MDA-1 | 9 | 2827078 | 2827078 | Missense_Mutation |
| KIAA1109 | MDA-1 | 4 | 123131078 | 123131078 | Splice_Site |
| KLF11 | MDA-1 | 2 | 10187822 | 10187822 | Missense_Mutation |
| KNTC1 | MDA-1 | 12 | 123087655 | 123087655 | Intron |
| KRT35 | MDA-1 | 17 | 39637094 | 39637094 | Missense_Mutation |
| KRTAP24-1 | MDA-1 | 21 | 31654701 | 31654701 | Missense_Mutation |
| LINC00207 | MDA-1 | 22 | 44966400 | 44966400 | lincRNA |
| LRIG2 | MDA-1 | 1 | 113661942 | 113661942 | Missense_Mutation |
| LRRC16B | MDA-1 | 14 | 24526377 | 24526377 | Splice_Site |
| LSM14B | MDA-1 | 20 | 60705322 | 60705322 | Missense_Mutation |
| MB21D2 | MDA-1 | 3 | 192516813 | 192516813 | Missense_Mutation |
| MCF2L2 | MDA-1 | 3 | 182923971 | 182923971 | Missense_Mutation |
| MED12L | MDA-1 | 3 | 151101587 | 151101587 | Silent |
| MIR1207 | MDA-1 | 8 | 129061462 | 129061462 | RNA |
| MLXIP | MDA-1 | 12 | 122618415 | 122618415 | Missense_Mutation |
| MSLNL | MDA-1 | 16 | 825490 | 825490 | Missense_Mutation |
| MST1 | MDA-1 | 3 | 49723393 | 49723393 | Missense_Mutation |
| MT1L | MDA-1 | 16 | 56651379 | 56651379 | RNA |
| MT3 | MDA-1 | 16 | 56624854 | 56624854 | Missense_Mutation |
| MTHFD2L | MDA-1 | 4 | 75091057 | 75091057 | Missense_Mutation |
| MUC17 | MDA-1 | 7 | 100675393 | 100675393 | Silent |
| MYO10 | MDA-1 | 5 | 16701732 | 16701732 | Missense_Mutation |
| NARS | MDA-1 | 18 | 55273918 | 55273918 | Missense_Mutation |
| NCBP1 | MDA-1 | 9 | 100409817 | 100409817 | Missense_Mutation |
| NCOR2 | MDA-1 | 12 | 124862913 | 124862913 | Silent |
| NDUFB10 | MDA-1 | 16 | 2013040 | 2013040 | IGR |
| NEK9 | MDA-1 | 14 | 75568358 | 75568358 | Splice_Site |
| NEU4 | MDA-1 | 2 | 242757729 | 242757729 | Silent |
| NFATC4 | MDA-1 | 14 | 24836781 | 24836781 | Intron |
| NKX2-6 | MDA-1 | 8 | 23563912 | 23563912 | Missense_Mutation |
| NNT | MDA-1 | 5 | 43651894 | 43651894 | Missense_Mutation |
| NPAT | MDA-1 | 11 | 108093355 | 108093355 | 5′UTR |
| NRXN1 | MDA-1 | 2 | 50574367 | 50574367 | Intron |
| NUMBL | MDA-1 | 19 | 41174010 | 41174010 | Missense_Mutation |
| OGFOD2 | MDA-1 | 12 | 123463489 | 123463489 | Missense_Mutation |
| ORAI3 | MDA-1 | 16 | 30965479 | 30965479 | Intron |
| OTUD7B | MDA-1 | 1 | 149907209 | 149907209 | IGR |
| OXSR1 | MDA-1 | 3 | 38207236 | 38207236 | De_novo_Start_OutOfFrame |
| PAFAH1B3 | MDA-1 | 19 | 42806354 | 42806354 | Intron |
| PALD1 | MDA-1 | 10 | 72285814 | 72285814 | Missense_Mutation |
| PAPPA | MDA-1 | 9 | 118997737 | 118997737 | Silent |
| PCDHGB3 | MDA-1 | 5 | 140778157 | 140778157 | Intron |
| PCID2 | MDA-1 | 13 | 113833282 | 113833282 | Missense_Mutation |
| PDE6A | MDA-1 | 5 | 149276036 | 149276036 | Silent |
| PI4KA | MDA-1 | 22 | 21165309 | 21165309 | Silent |
| PIK3C2A | MDA-1 | 11 | 17167386 | 17167386 | Intron |
| PKD1L1 | MDA-1 | 7 | 47921683 | 47921683 | Splice_Site |
| PKD1L3 | MDA-1 | 16 | 71976911 | 71976911 | RNA |
| PKD2L2 | MDA-1 | 5 | 137275656 | 137275656 | 3′UTR |
| PMEL | MDA-1 | 12 | 56351763 | 56351763 | Missense_Mutation |
| POU5F1 | MDA-1 | 6 | 31130524 | 31130524 | IGR |
| PPM1D | MDA-1 | 17 | 53677718 | 58677718 | 5′UTR |
| PPP1R1B | MDA-1 | 17 | 37783643 | 37783643 | 5′UTR |
| PPP4R1 | MDA-1 | 18 | 9583132 | 9583132 | Missense_Mutation |
| PRKD3 | MDA-1 | 2 | 37501808 | 37501808 | Silent |
| PRR24 | MDA-1 | 19 | 47778307 | 47778307 | Missense_Mutation |
| PSD4 | MDA-1 | 2 | 113943416 | 113943416 | Intron |
| RAB3GAP2 | MDA-1 | 1 | 220320914 | 220320914 | IGR |
| RAC2 | MDA-1 | 22 | 37637174 | 37637174 | Intron |
| RAPGEF6 | MDA-1 | 5 | 130788792 | 130788792 | Missense_Mutation |
| RASGRF1 | MDA-1 | 15 | 79254437 | 79254437 | 3′UTR |
| RP11-54D18.3 | MDA-1 | 9 | 15047904 | 15047904 | RNA |
| RP11-794P6.2 | MDA-1 | 11 | 111327280 | 111327280 | Intron |
| RTKN | MDA-1 | 2 | 74657626 | 74657626 | Missense_Mutation |
| RTN3 | MDA-1 | 11 | 63521144 | 63521144 | Silent |
| SDCCAG8 | MDA-1 | 1 | 243433414 | 243433414 | Silent |
| SH3BGR | MDA-1 | 21 | 40887097 | 40887097 | 3′UTR |
| SLC13A3 | MDA-1 | 20 | 45217841 | 45217841 | Missense_Mutation |
| SLC17A4 | MDA-1 | 6 | 25778222 | 25778222 | Missense_Mutation |
| SLC25A15 | MDA-1 | 13 | 41373522 | 41373522 | Intron |
| SLIRP | MDA-1 | 14 | 78189606 | 78189606 | IGR |
| SON | MDA-1 | 21 | 34926768 | 34926768 | Missense_Mutation |
| SORCS2 | MDA-1 | 4 | 7194810 | 7194810 | Missense_Mutation |
| SPAG6 | MDA-1 | 10 | 22680829 | 22680829 | Missense_Mutation |
| SRRM2 | MDA-1 | 16 | 2802811 | 2802811 | 5′UTR |
| SUSD2 | MDA-1 | 22 | 24580150 | 24580150 | Silent |
| SYT14 | MDA-1 | 1 | 210337320 | 210337320 | IGR |
| SZT2 | MDA-1 | 1 | 43916570 | 43916570 | 3′UTR |
| TDRD6 | MDA-1 | 6 | 46657514 | 46657514 | Missense_Mutation |
| TDRD6 | MDA-1 | 6 | 46656177 | 46656177 | Silent |
| THBS1 | MDA-1 | 15 | 39885328 | 39885328 | Silent |
| TIE1 | MDA-1 | 1 | 43779586 | 43779586 | Missense_Mutation |
| TIE1 | MDA-1 | 1 | 43770936 | 43770936 | Missense_Mutation |
| TLR4 | MDA-1 | 9 | 120476185 | 120476185 | Nonsense_Mutation |
| TMED4 | MDA-1 | 7 | 44619029 | 44619029 | 3′UTR |
| TNFAIP8 | MDA-1 | 5 | 118690924 | 118690924 | 5′UTR |
| TNIK | MDA-1 | 3 | 170811733 | 170811733 | Silent |
| TRPC1 | MDA-1 | 3 | 142443349 | 142443349 | 5′UTR |
| TSPAN4 | MDA-1 | 11 | 850346 | 850346 | Silent |
| UBA7 | MDA-1 | 3 | 49851352 | 49851352 | 5′UTR |
| USP37 | MDA-1 | 2 | 219423357 | 219423357 | Missense_Mutation |
| USP4 | MDA-1 | 3 | 49365185 | 49365185 | Silent |
| VPS4A | MDA-1 | 16 | 69352811 | 69352811 | IGR |
| VWA8 | MDA-1 | 13 | 42179335 | 42179335 | Missense_Mutation |
| WDR78 | MDA-1 | 1 | 67390719 | 67390719 | 5′Flank |
| WIPF1 | MDA-1 | 2 | 175426977 | 175426977 | 3′UTR |
| YPEL4 | MDA-1 | 11 | 57414510 | 57414510 | Missense_Mutation |
| ZCCHC4 | MDA-1 | 4 | 25334843 | 25334843 | Missense_Mutation |
| ZFHX4 | MDA-1 | 8 | 77763768 | 77763768 | Silent |
| ZMYM6 | MDA-1 | 1 | 35472669 | 35472669 | Missense_Mutation |
| ZNF215 | MDA-1 | 11 | 6977338 | 6977338 | Missense_Mutation |
| ZNF219 | MDA-1 | 14 | 21567088 | 21567088 | 5′UTR |
| ZNF28 | MDA-1 | 19 | 53303413 | 53303413 | IGR |
| ZNF408 | MDA-1 | 11 | 46726678 | 46726678 | Silent |
| ZNF408 | MDA-1 | 11 | 46726773 | 46726773 | Missense_Mutation |
| ZNF408 | MDA-1 | 11 | 46726774 | 46726774 | Silent |
| ZNF517 | MDA-1 | 8 | 146033535 | 146033535 | Missense_Mutation |
| ZNF560 | MDA-1 | 19 | 9577708 | 9577708 | Missense_Mutation |
| ZNF696 | MDA-1 | 8 | 144378499 | 144378499 | Silent |
| ZNF883 | MDA-1 | 9 | 115760450 | 115760450 | lincRNA |
| loss_17p | MDA-1 | 17 | 11268 | 11268 | arm_level |
| DIS3 | MDA-1 | 13 | 73346034 | 73346034 | Splice_Site |
| FLG | MDA-1 | 1 | 152285533 | 152285533 | Missense_Mutation |
| MTOR | MDA-1 | 1 | 11273600 | 11273600 | Silent |
| DYSF | MDA-1 | 2 | 71816674 | 71816674 | Intron |
| C1orf141 | MDA-1 | 1 | 67591462 | 67591462 | In_Frame_Del |
| NFKBIA | MDA-1 | 14 | 35871144 | 35871144 | 3′UTR |
| COL1A1 | MDA-1 | 17 | 48268874 | 48268874 | Intron |
| SIPA1 | MDA-1 | 11 | 65417544 | 65417544 | Intron |
| DPAGT1 | MDA-1 | 11 | 118972984 | 118972984 | 5′UTR |
| PRPSAP1 | MDA-1 | 17 | 74303641 | 74303641 | IGR |
| FN3K | MDA-1 | 17 | 80707026 | 80707026 | Intron |
| MGP | MDA-1 | 12 | 15037257 | 15037257 | Intron |
| GUCY2D | MDA-1 | 17 | 7906495 | 7906495 | In_Frame_Del |
| LMBR1 | MDA-1 | 7 | 156549023 | 156549023 | Intron |
| COL6A1 | MDA-1 | 21 | 47407300 | 47407300 | Intron |
| TRPM4 | MDA-1 | 19 | 49669256 | 49669256 | Intron |
| RP11-295K3.1 | MDA-1 | 11 | 1775188 | 1775188 | Intron |
| AE000661.50 | MDA-1 | 14 | 22887651 | 22887651 | lincRNA |
| C17orf66 | MDA-1 | 17 | 34185385 | 34185385 | Intron |
| CLEC17A | MDA-1 | 19 | 14706064 | 14706064 | Intron |
| EXOSC9 | MDA-1 | 4 | 122731087 | 122731087 | Intron |
| FARP2 | MDA-1 | 2 | 242381871 | 242381871 | Intron |
| SON | MDA-1 | 21 | 34924280 | 34924280 | In_Frame_Del |
| NUDT17 | MDA-1 | 1 | 145588656 | 145588656 | Intron |
| AKAP12 | MDA-1 | 6 | 151672689 | 151672689 | Missense_Mutation |
| PLEKHG1 | MDA-1 | 6 | 151161157 | 151161157 | Missense_Mutation |
| SYNE1 | MDA-1 | 6 | 152702311 | 152702311 | Missense_Mutation |
| HORMAD2 | MDA-1 | 22 | 30494844 | 30494844 | Frame_Shift_Del |
| ATP8B2 | MDA-1 | 1 | 154315677 | 154315677 | Silent |
| CTD-2090I13.1 | MDA-1 | 1 | 227618307 | 227618307 | lincRNA |
| LYST | MDA-1 | 1 | 235901547 | 235901547 | Intron |
| EXOC6B | MDA-1 | 2 | 72723734 | 72723734 | Intron |
| ADAMTS13 | MDA-1 | 9 | 136303408 | 136303408 | Missense_Mutation |
| ADRA1A | MDA-1 | 8 | 26721824 | 26721824 | Silent |
| AGAP1 | MDA-1 | 2 | 236708124 | 236708124 | Silent |
| ALOX15B | MDA-1 | 17 | 7942858 | 7942858 | Missense_Mutation |
| ALOX15B | MDA-1 | 17 | 7942611 | 7942611 | Silent |
| ASXL3 | MDA-1 | 18 | 31241641 | 31241641 | Silent |
| BAI3 | MDA-1 | 6 | 70042825 | 70042825 | Missense_Mutation |
| BMP2 | MDA-1 | 20 | 6759416 | 6759416 | Missense_Mutation |
| C1orf228 | MDA-1 | 1 | 45163892 | 45163892 | Missense_Mutation |
| C6orf223 | MDA-1 | 6 | 43968844 | 43968844 | Missense_Mutation |
| CDH23 | MDA-1 | 10 | 73434870 | 73434870 | Splice_Site |
| CHST9 | MDA-1 | 18 | 24496858 | 24496858 | Missense_Mutation |
| CHSY3 | MDA-1 | 5 | 129520406 | 129520406 | Missense_Mutation |
| CLCN6 | MDA-1 | 1 | 11900298 | 11900298 | 3′UTR |
| CLCN6 | MDA-1 | 1 | 11900279 | 11900279 | Nonstop_Mutation |
| DMXL1 | MDA-1 | 5 | 118539121 | 118539121 | Missense_Mutation |
| EDC3 | MDA-1 | 15 | 74963997 | 74963997 | Missense_Mutation |
| FAM160B1 | MDA-1 | 10 | 116623340 | 116623340 | 3′UTR |
| GIMAP7 | MDA-1 | 7 | 150217208 | 150217208 | Missense_Mutation |
| HERC2 | MDA-1 | 15 | 28525392 | 28525392 | Missense_Mutation |
| HIST1H2AC | MDA-1 | 6 | 26124770 | 26124770 | Missense_Mutation |
| HTR7 | MDA-1 | 10 | 92508591 | 92508591 | 3′UTR |
| IGSF9B | MDA-1 | 11 | 133792571 | 133792571 | Missense_Mutation |
| JUNB | MDA-1 | 19 | 12902748 | 12902748 | Missense_Mutation |
| KIRREL | MDA-1 | 1 | 158047907 | 158047907 | Missense_Mutation |
| MAGI2 | MDA-1 | 7 | 78150857 | 78150857 | 5′UTR |
| NLRP5 | MDA-1 | 19 | 56565030 | 56565030 | Missense_Mutation |
| OAS2 | MDA-1 | 12 | 113447025 | 113447025 | Missense_Mutation |
| PAX9 | MDA-1 | 14 | 37132532 | 37132532 | Silent |
| PIWIL2 | MDA-1 | 8 | 22168789 | 22168789 | Silent |
| PSG9 | MDA-1 | 19 | 43763145 | 43763145 | Silent |
| SEC11C | MDA-1 | 18 | 56816778 | 56816778 | Missense_Mutation |
| SEC14L3 | MDA-1 | 22 | 30860845 | 30860845 | Missense_Mutation |
| SLC18A3 | MDA-1 | 10 | 50819048 | 50819048 | Missense_Mutation |
| SLC26A9 | MDA-1 | 1 | 205887995 | 205887995 | Silent |
| TMEM91 | MDA-1 | 19 | 41869348 | 41869348 | Intron |
| USP8 | MDA-1 | 15 | 50782476 | 50782476 | Missense_Mutation |
| XRN1 | MDA-1 | 3 | 142119314 | 142119314 | Missense_Mutation |
| XRRA1 | MDA-1 | 11 | 74556315 | 74556315 | Splice_Site |
| ZNF318 | MDA-1 | 6 | 43336757 | 43336757 | Missense_Mutation |
| ATM | MDA-1 | 11 | 108203621 | 108203621 | Nonsense_Mutation |
| ATM | MDA-1 | 11 | 108206648 | 108206648 | Missense_Mutation |
| SERPINB13 | MDA-1 | 18 | 61264391 | 61264391 | Missense_Mutation |
| SF3B1 | MDA-1 | 2 | 198266834 | 198266834 | Missense_Mutation |
| TP53 | MDA-1 | 17 | 7577539 | 7577539 | Missense_Mutation |
| gain_1q23.3 | MDA-1 | 1 | 155899084 | 155899084 | focal_level |
| gain_TERT | MDA-1 | 5 | 0 | 0 | focal_level |
| SFSWAP | MDA-1 | 12 | 132262624 | 132262624 | In_Frame_Ins |
| loss_10q25.1 | MDA-1 | 10 | 0 | 0 | focal_level |
| gain_6p21.1 | MDA-1 | 6 | 0 | 0 | focal_level |
| gain_22q11.21 | MDA-1 | 22 | 0 | 0 | focal_level |
| gain_MCL1 | MDA-1 | 1 | 0 | 0 | focal_level |
| gain_9p24.2 | MDA-1 | 9 | 0 | 0 | focal_level |
| gain_11q13.2 | MDA-1 | 11 | 0 | 0 | focal_level |
| AMOTL1 | MDA-1 | 11 | 94532695 | 94532695 | Silent |
| CADPS2 | MDA-1 | 7 | 122056192 | 122056192 | Missense_Mutation |
| GAS2L3 | MDA-1 | 12 | 101012342 | 101012342 | Missense_Mutation |
| IDO1 | MDA-1 | 8 | 39782839 | 39782839 | Missense_Mutation |
| KBTBD11 | MDA-1 | 8 | 1949784 | 1949784 | Silent |
| NPR2 | MDA-1 | 9 | 35811698 | 35811698 | IGR |
| OR10K1 | MDA-1 | 1 | 158435618 | 158435618 | Missense_Mutation |
| PGBD1 | MDA-1 | 6 | 28269532 | 28269532 | Missense_Mutation |
| PLCH1 | MDA-1 | 3 | 155199029 | 155199029 | Missense_Mutation |
| PSMD9 | MDA-1 | 12 | 122326731 | 122326731 | 5′UTR |
| TAPT1 | MDA-1 | 4 | 16228907 | 16228907 | 5′Flank |
| TP53 | MDA-1 | 17 | 7578263 | 7578263 | Nonsense_Mutation |
| TSC2 | MDA-1 | 16 | 2106716 | 2106716 | Silent |
| SLC4A4 | MDA-1 | 4 | 72102269 | 72102269 | Intron |
| BAZ2A | MDA-1 | 12 | 56995184 | 56995184 | Frame_Shift_Ins |
| gain_KRAS | MDA-1 | 12 | 25243120 | 25243120 | focal_level |
| loss_6q | MDA-1 | 6 | 89926979 | 89926979 | arm_level |
| DRG2 | MDA-1 | 17 | 17991306 | 17991306 | 5′Flank |
| homdel_2q37.3 | MDA-1 | 2 | 242135224 | 242135224 | focal_level |
| gain_AKT3 | MDA-1 | 1 | 244999804 | 244999804 | focal_level |
| AP000275.65 | MDA-1 | 21 | 33975570 | 33975570 | Frame_Shift_Ins |
| ASCC3 | MDA-1 | 6 | 101248307 | 101248307 | Silent |
| CTNNA2 | MDA-1 | 2 | 80136918 | 80136918 | Missense_Mutation |
| DST | MDA-1 | 6 | 56330919 | 56330919 | Missense_Mutation |
| FAM179B | MDA-1 | 14 | 45465138 | 45465138 | Intron |
| MTERFD2 | MDA-1 | 2 | 242036571 | 242036571 | Intron |
| PNLDC1 | MDA-1 | 6 | 160222206 | 160222206 | Missense_Mutation |
| RASSF8 | MDA-1 | 12 | 26217538 | 26217538 | Missense_Mutation |
| RDH16 | MDA-1 | 12 | 57345813 | 57345813 | Nonstop_Mutation |
| SYNM | MDA-1 | 15 | 99678194 | 99678194 | IGR |
| TCTN2 | MDA-1 | 12 | 124172597 | 124172597 | Splice_Site |
| TRBV25-1 | MDA-1 | 7 | 142378971 | 142378971 | RNA |
| TRIM24 | MDA-1 | 7 | 138210057 | 138210057 | Missense_Mutation |
| gain_MDM4 | MDA-1 | 1 | 0 | 0 | focal_level |
| gain_11q14.1 | MDA-1 | 11 | 0 | 0 | focal_level |
| loss_9p13.1 | MDA-1 | 9 | 0 | 0 | focal_level |
| gain_19q13.42 | MDA-1 | 19 | 0 | 0 | focal_level |
| gain_11q22.2 | MDA-1 | 11 | 0 | 0 | focal_level |
| gain_CCND1 | MDA-1 | 11 | 0 | 0 | focal_level |
| gain_8q21.13 | MDA-1 | 8 | 0 | 0 | focal_level |
| MTMR3 | MDA-1 | 22 | 30384504 | 30384504 | 5′UTR |
| MYPN | MDA-1 | 10 | 69961683 | 69961683 | Silent |
| SPIDR | MDA-1 | 8 | 48585972 | 48585972 | Intron |
| VNN1 | MDA-1 | 6 | 133014444 | 133014444 | Missense_Mutation |
| ERCC2 | MDA-1 | 19 | 45860751 | 45860751 | Missense_Mutation |
| homdel_19q13.32 | MDA-1 | 19 | 45643447 | 45643447 | focal_level |
| gain_NKX2-1 | MDA-1 | 14 | 0 | 0 | focal_level |
| homdel_3p26.3 | MDA-1 | 3 | 0 | 0 | focal_level |
| homdel_11p15.5 | MDA-1 | 11 | 372620 | 372620 | focal_level |
| gain_5q35.3 | MDA-1 | 5 | 0 | 0 | focal_level |
| loss_16q23.1 | MDA-1 | 16 | 0 | 0 | focal_level |
| gain_10p15.1 | MDA-1 | 10 | 93514 | 93514 | focal_level |
| loss_1p36.23 | MDA-1 | 1 | 0 | 0 | focal_level |
| homdel_STK11 | MDA-1 | 19 | 66207 | 66207 | focal_level |
| gain_ZNF217 | MDA-1 | 20 | 0 | 0 | focal_level |
| homdel_BRCA1 | MDA-1 | 17 | 0 | 0 | focal_level |
| gain_3q29 | MDA-1 | 3 | 184035626 | 184035626 | focal_level |
| FAM13C | MDA-1 | 10 | 61120839 | 61120839 | Intron |
| IGHV1-24 | MDA-1 | 14 | 106733177 | 106733177 | RNA |
| NDUFA9 | MDA-1 | 12 | 4768297 | 4768297 | Missense_Mutation |
| PABPC4L | MDA-1 | 4 | 135121930 | 135121930 | Missense_Mutation |
| TDRD9 | MDA-1 | 14 | 104493257 | 104493257 | Missense_Mutation |
| homdel_16p13.3 | MDA-1 | 16 | 64355 | 64355 | focal_level |
| homdel_17q25.3 | MDA-1 | 17 | 78933941 | 78933941 | focal_level |
| loss_9q34.3 | MDA-1 | 9 | 134738488 | 134738488 | focal_level |
| gain_E2F3 | MDA-1 | 6 | 17600996 | 17600996 | focal_level |
| loss_19q | MDA-1 | 19 | 26500001 | 26500001 | arm_level |
| loss_19p | MDA-1 | 19 | 7794161 | 7794161 | arm_level |
| loss_15q | MDA-1 | 15 | 20169963 | 20169963 | arm_level |
| homdel_12q24.33 | MDA-1 | 12 | 124158259 | 124158259 | focal_level |
| gain_17q11.2 | MDA-1 | 17 | 22023424 | 22023424 | focal_level |
| gain_13q34 | MDA-1 | 13 | 111009859 | 111009859 | focal_level |
| gain_13q | MDA-1 | 13 | 28636105 | 28636105 | arm_level |
| loss_18p | MDA-1 | 18 | 3135636 | 3135636 | arm_level |
| loss_1q44 | MDA-1 | 1 | 244999804 | 244999804 | focal_level |
| homdel_4p16.3 | MDA-1 | 4 | 502688 | 502688 | focal_level |
| loss_APC | MDA-1 | 5 | 98192256 | 98192256 | focal_level |
| gain_ERBB2 | MDA-1 | 17 | 36352492 | 36352492 | focal_level |
| gain_9p | MDA-1 | 9 | 14783 | 14783 | arm_level |
| homdel_10q26.3 | MDA-1 | 10 | 129839197 | 129839197 | focal_level |
| gain_NEDD9 | MDA-1 | 6 | 203616 | 203616 | focal_level |
| homdel_21q22.3 | MDA-1 | 21 | 41032953 | 41032953 | focal_level |
| homdel_16q23.1 | MDA-1 | 16 | 74908224 | 74908224 | focal_level |
| homdel_IKZF2 | MDA-1 | 2 | 211421050 | 211421050 | focal_level |
| homdel_1p36.23 | MDA-1 | 1 | 1391715 | 1391715 | focal_level |
| loss_22q | MDA-1 | 22 | 19026506 | 19026506 | arm_level |
| AMOTL2 | MDA-2 | 3 | 134090204 | 134090204 | Silent |
| ANAPC4 | MDA-2 | 4 | 25382101 | 25382101 | Splice_Site |
| ANKRD11 | MDA-2 | 16 | 89341518 | 89341518 | Nonsense_Mutation |
| BIRC3 | MDA-2 | 11 | 102207790 | 102207790 | Missense_Mutation |
| C8orf48 | MDA-2 | 8 | 13424852 | 13424852 | Nonsense_Mutation |
| CDH11 | MDA-2 | 16 | 65026835 | 65026835 | Missense_Mutation |
| COL6A5 | MDA-2 | 3 | 130188150 | 130188150 | Silent |
| CORO1A | MDA-2 | 16 | 30198004 | 30198004 | Missense_Mutation |
| CPEB2 | MDA-2 | 4 | 15005914 | 15005914 | Silent |
| DIRAS3 | MDA-2 | 1 | 68512864 | 68512864 | Silent |
| DNAH9 | MDA-2 | 17 | 11648208 | 11648208 | Missense_Mutation |
| EGR2 | MDA-2 | 10 | 64573332 | 64573332 | Missense_Mutation |
| ELP3 | MBA-2 | 8 | 28019514 | 28019514 | Splice_Site |
| ENKD1 | MDA-2 | 16 | 67696094 | 67696094 | IGR |
| ENO2 | MDA-2 | 12 | 7026829 | 7026829 | Intron |
| FLNB | MDA-2 | 3 | 57994391 | 57994391 | Missense_Mutation |
| FPR2 | MDA-2 | 19 | 52272264 | 52272264 | Missense_Mutation |
| GABARAPL2 | MDA-2 | 16 | 75601950 | 75601950 | Missense_Mutation |
| GPR133 | MDA-2 | 12 | 131488811 | 131488811 | Nonsense_Mutation |
| HCN1 | MDA-2 | 5 | 45396641 | 45396641 | Missense_Mutation |
| IGHMBP2 | MDA-2 | 11 | 68697880 | 68697880 | Intron |
| IKBIP | MDA-2 | 12 | 99020023 | 99020023 | Silent |
| INTU | MDA-2 | 4 | 128608937 | 128608937 | Missense_Mutation |
| IRS2 | MDA-2 | 13 | 110436229 | 110436229 | Silent |
| KIAA0196 | MDA-2 | 8 | 126073352 | 126073352 | Missense_Mutation |
| MED7 | MDA-2 | 5 | 156566010 | 156566010 | Missense_Mutation |
| MICAL3 | MDA-2 | 22 | 18387572 | 18387572 | Missense_Mutation |
| MTMR12 | MDA-2 | 5 | 32263277 | 32263277 | Missense_Mutation |
| OBSL1 | MDA-2 | 2 | 220417648 | 220417648 | Silent |
| PCGF5 | MDA-2 | 10 | 93000303 | 93000303 | Missense_Mutation |
| SCO1 | MDA-2 | 17 | 10584508 | 10584508 | Silent |
| SLC12A4 | MDA-2 | 16 | 67980168 | 67980168 | Missense_Mutation |
| SLC26A8 | MDA-2 | 6 | 35987370 | 35987370 | Missense_Mutation |
| SNED1 | MDA-2 | 2 | 241988534 | 241988534 | Missense_Mutation |
| SOX5 | MDA-2 | 12 | 23793755 | 23793755 | Silent |
| SYNE1 | MDA-2 | 6 | 152776560 | 152776560 | Splice_Site |
| TACR2 | MDA-2 | 10 | 71164666 | 71164666 | Silent |
| TBX6 | MDA-2 | 16 | 30100374 | 30100374 | Missense_Mutation |
| TDRD6 | MDA-2 | 6 | 46656097 | 46656097 | Missense_Mutation |
| TMEM175 | MDA-2 | 4 | 947036 | 947036 | Missense_Mutation |
| UBA52 | MDA-2 | 19 | 18686046 | 18686046 | 3′UTR |
| ZNF408 | MDA-2 | 11 | 46722598 | 46722598 | Start_Codon_SNP |
| ATM | MDA-2 | 11 | 108199835 | 108199835 | Missense_Mutation |
| IRF4 | MDA-2 | 6 | 394950 | 394950 | Missense_Mutation |
| IRF4 | MDA-2 | 6 | 394951 | 394951 | Missense_Mutation |
| TP53 | MDA-2 | 17 | 7578406 | 7578406 | Missense_Mutation |
| gain_21q | MDA-2 | 21 | 0 | 0 | arm_level |
| homdel_17q25.3 | MDA-2 | 17 | 76692090 | 76692090 | focal_level |
| homdel_22q13.32 | MDA-2 | 22 | 42540374 | 42540374 | focal_level |
| homdel_19q13.32 | MDA-2 | 19 | 40400564 | 40400564 | focal_level |
| homdel_14q32.33 | MDA-2 | 14 | 102323122 | 102323122 | focal_level |
| loss_18q23 | MDA-2 | 18 | 74962837 | 74962837 | focal_level |
| homdel_17p13.3 | MDA-2 | 17 | 11268 | 11268 | focal_level |
| gain_1q23.3 | MDA-2 | 1 | 153631640 | 153631640 | focal_level |
| NLGN1 | MDA-2 | 3 | 173993271 | 173993271 | Frame_Shift_Del |
| gain_22q11.21 | MDA-2 | 22 | 19466599 | 19466599 | focal_level |
| gain_MDM4 | MDA-2 | 1 | 204234120 | 204234120 | focal_level |
| homdel_10q25.1 | MDA-2 | 10 | 0 | 0 | focal_level |
| homdel_STK11 | MDA-2 | 19 | 1108895 | 1108895 | focal_level |
| loss_12q24.33 | MDA-2 | 12 | 0 | 0 | focal_level |
| homdel_19q13.43 | MDA-2 | 19 | 54819095 | 54819095 | focal_level |
| homdel_2q37.3 | MDA-2 | 2 | 0 | 0 | focal_level |
| gain_ZNF217 | MDA-2 | 20 | 0 | 0 | focal_level |
| gain_E2F3 | MDA-2 | 6 | 203616 | 203616 | focal_level |
| loss_19p | MDA-2 | 19 | 66207 | 66207 | arm_level |
| homdel_7p22.3 | MDA-2 | 7 | 193501 | 193501 | focal_level |
| gain_4p | MDA-2 | 4 | 6578378 | 6578378 | arm_level |
| gain_12q | MDA-2 | 12 | 35800001 | 35800001 | arm_level |
| gain_MDM2 | MDA-2 | 12 | 57957163 | 57957163 | focal_level |
| gain_NEDD9 | MDA-2 | 6 | 203616 | 203616 | focal_level |
| loss_5q35.3 | MDA-2 | 5 | 179538518 | 179538518 | focal_level |
| loss_20p | MDA-2 | 20 | 68379 | 68379 | arm_level |
| gain_8p | MDA-2 | 8 | 0 | 0 | arm_level |
| gain_CCND1 | MDA-2 | 11 | 0 | 0 | focal_level |
| homdel_21q22.3 | MDA-2 | 21 | 46078042 | 46078042 | focal_level |
| homdel_16q23.1 | MDA-2 | 16 | 0 | 0 | focal_level |
| gain_18q | MDA-2 | 18 | 0 | 0 | arm_level |
| loss_16p | MDA-2 | 16 | 15680630 | 15680630 | arm_level |
| loss_11q23.1 | MDA-2 | 11 | 82444131 | 82444131 | focal_level |
| NELL1 | MDA-2 | 11 | 20699494 | 20699494 | Frame_Shift_Del |
| loss_22q | MDA-2 | 22 | 16157621 | 16157621 | arm_level |
| gain_17q24.2 | MDA-2 | 17 | 0 | 0 | focal_level |
| gain_IGF1R | MDA-2 | 15 | 90347519 | 90347519 | focal_level |
| loss_MLL3 | MDA-2 | 7 | 0 | 0 | focal_level |
| homdel_1p36.23 | MDA-2 | 1 | 12175 | 12175 | focal_level |
| gain_NKX2-1 | MDA-2 | 14 | 0 | 0 | focal_level |
| gain_13q | MDA-2 | 13 | 0 | 0 | arm_level |
| loss_15q15.1 | MDA-2 | 15 | 0 | 0 | focal_level |
| gain_CDK4 | MDA-2 | 12 | 57957163 | 57957163 | focal_level |
| loss_8p21.2 | MDA-2 | 8 | 0 | 0 | focal_level |
| gain_10p15.1 | MDA-2 | 10 | 0 | 0 | focal_level |
| gain_11q14.1 | MDA-2 | 11 | 0 | 0 | focal_level |
| gain_7q36.3 | MDA-2 | 7 | 154720339 | 154720339 | focal_level |
| gain_12p | MDA-2 | 12 | 19592927 | 19592927 | arm_level |
| homdel_10q26.3 | MDA-2 | 10 | 134062573 | 134062573 | focal_level |
| gain_12p13.33 | MDA-2 | 12 | 78865 | 78865 | focal_level |
| gain_9p24.2 | MDA-2 | 9 | 0 | 0 | focal_level |
| homdel_9q34.3 | MDA-2 | 9 | 136081283 | 136081283 | focal_level |
| homdel_4p16.3 | MDA-2 | 4 | 59385 | 59385 | focal_level |
| gain_KRAS | MDA-2 | 12 | 19592927 | 19592927 | focal_level |
| homdel_5q35.3 | MDA-2 | 5 | 0 | 0 | focal_level |
| gain_3q | MDA-2 | 3 | 130313169 | 130313169 | arm_level |
| loss_17p | MDA-2 | 17 | 11268 | 11268 | arm_level |
| loss_4p | MDA-2 | 4 | 6578378 | 6578378 | arm_level |
| gain_5q | MDA-2 | 5 | 69736634 | 69736634 | arm_level |
| gain_11q13.2 | MDA-2 | 11 | 0 | 0 | focal_level |
| homdel_15q15.1 | MDA-2 | 15 | 20169963 | 20169963 | focal_level |
| AGXT2 | MDA-3 | 5 | 35037047 | 35037047 | Splice_Site |
| BCL11B | MDA-3 | 14 | 99640472 | 99640472 | 3′UTR |
| BTBD19 | MDA-3 | 1 | 45279566 | 45279566 | 3′UTR |
| CAPRIN2 | MDA-3 | 12 | 30881755 | 30881755 | Nonsense_Mutation |
| CDH2 | MDA-3 | 18 | 25565083 | 25565083 | Missense_Mutation |
| CEP112 | MDA-3 | 17 | 64125931 | 64125931 | Missense_Mutation |
| CPN1 | MDA-3 | 10 | 101823394 | 101823394 | Missense_Mutation |
| DDX49 | MDA-3 | 19 | 19038937 | 19038937 | Splice_Site |
| DNAH17 | MDA-3 | 17 | 76456002 | 76456002 | Missense_Mutation |
| DTNA | MDA-3 | 18 | 32392010 | 32392010 | Missense_Mutation |
| HNF1B | MDA-3 | 17 | 36099531 | 36099531 | Silent |
| HNF4A | MDA-3 | 20 | 43053034 | 43053034 | Intron |
| HTRA3 | MDA-3 | 4 | 8288312 | 8288312 | Silent |
| IL1R2 | MDA-3 | 2 | 102641126 | 102641126 | Missense_Mutation |
| ITGA4 | MDA-3 | 2 | 182402403 | 182402403 | IGR |
| LAPTM5 | MDA-3 | 1 | 31230602 | 31230602 | De_novo_Start_OutOfFrame |
| MAN1B1 | MDA-3 | 9 | 140002966 | 140002966 | Missense_Mutation |
| NBPF22P | MDA-3 | 5 | 85578423 | 85578423 | RNA |
| NCKAP1 | MDA-3 | 2 | 183800091 | 183800091 | Missense_Mutation |
| NIPAL4 | MDA-3 | 5 | 156894089 | 156894089 | Missense_Mutation |
| NRXN2 | MDA-3 | 11 | 64435958 | 64435958 | Missense_Mutation |
| OBSCN | MDA-3 | 1 | 228399849 | 228399849 | Missense_Mutation |
| OR4C6 | MDA-3 | 11 | 55433623 | 55433623 | 3′UTR |
| OR4C6 | MDA-3 | 11 | 55433624 | 55433624 | 3′UTR |
| OR4N5 | MDA-3 | 14 | 20612441 | 20612441 | Nonsense_Mutation |
| OR52N2 | MDA-3 | 11 | 5842456 | 5842456 | Silent |
| P4HA3 | MDA-3 | 11 | 74022497 | 74022497 | Missense_Mutation |
| PCSK2 | MDA-3 | 20 | 17462397 | 17462397 | Silent |
| PDCD11 | MDA-3 | 10 | 105184926 | 105184926 | Silent |
| PDE6C | MDA-3 | 10 | 95372819 | 95372819 | Missense_Mutation |
| PDE6H | MDA-3 | 12 | 15131080 | 15131080 | Splice_Site |
| PNOC | MDA-3 | 8 | 28186755 | 28186755 | Silent |
| POU5F1 | MDA-3 | 6 | 31138427 | 31138427 | Intron |
| PRKAA2 | MDA-3 | 1 | 57169881 | 57169881 | Silent |
| RBL2 | MDA-3 | 16 | 53503957 | 53503957 | Missense_Mutation |
| RPS7 | MDA-3 | 2 | 3625354 | 3625354 | Missense_Mutation |
| SH3TC2 | MDA-3 | 5 | 148384418 | 148384418 | Silent |
| SLCO3A1 | MDA-3 | 15 | 92663804 | 92663804 | Silent |
| SNAP25 | MDA-3 | 20 | 10277587 | 10277587 | Missense_Mutation |
| SPATA17 | MDA-3 | 1 | 217915399 | 217915399 | Missense_Mutation |
| SYNPO2 | MDA-3 | 4 | 119978776 | 119978776 | Missense_Mutation |
| SYT3 | MDA-3 | 19 | 51140536 | 51140536 | Missense_Mutation |
| TMEM222 | MDA-3 | 1 | 27662013 | 27662013 | 3′UTR |
| TRIP10 | MDA-3 | 19 | 6741049 | 6741049 | Missense_Mutation |
| VPS13B | MDA-3 | 8 | 100147957 | 100147957 | Missense_Mutation |
| VWDE | MDA-3 | 7 | 12409877 | 12409877 | Silent |
| WDFY3 | MDA-3 | 4 | 85729561 | 85729561 | Missense_Mutation |
| ZCCHC10 | MDA-3 | 5 | 132362200 | 132362200 | Silent |
| loss_11q23.1 | MDA-3 | 11 | 103780484 | 103780484 | focal_level |
| SF3B1 | MDA-3 | 2 | 198267361 | 198267361 | Missense_Mutation |
| gain_MCL1 | MDA-3 | 1 | 0 | 0 | focal_level |
| CYB5RL | MDA-3 | 1 | 54640457 | 54640457 | Frame_Shift_Del |
| gain_8q | MDA-3 | 8 | 54142123 | 54142123 | arm_level |
| homdel_STK11 | MDA-3 | 19 | 0 | 0 | focal_level |
| TP53 | MDA-3 | 17 | 7578480 | 7578480 | In_Frame_Del |
| homdel_APC | MDA-3 | 5 | 0 | 0 | focal_level |
| gain_IGF1R | MDA-3 | 15 | 0 | 0 | focal_level |
| loss_14q32.33 | MDA-3 | 14 | 106330010 | 106330010 | focal_level |
| loss_8p | MDA-3 | 8 | 417733 | 417733 | arm_level |
| homdel_14q32.33 | MDA-3 | 14 | 0 | 0 | focal_level |
| gain_11q22.2 | MDA-3 | 11 | 0 | 0 | focal_level |
| homdel_RB1 | MDA-3 | 13 | 0 | 0 | focal_level |
| loss_19q | MDA-3 | 19 | 0 | 0 | arm_level |
| loss_19p | MDA-3 | 19 | 0 | 0 | arm_level |
| loss_6p | MDA-3 | 6 | 0 | 0 | arm_level |
| loss_3p26.3 | MDA-3 | 3 | 0 | 0 | focal_level |
| loss_17p | MDA-3 | 17 | 11268 | 11268 | arm_level |
| homdel_15q15.1 | MDA-3 | 15 | 35045126 | 35045126 | focal_level |
| EIF4A1 | MDA-3 | 17 | 7474085 | 7474085 | 5′Flank |
| Unknown | DFCI-5243 | 13 | 114453897 | 114453897 | IGR |
| AC015849.16 | DFCI-5243 | 17 | 34235657 | 34235657 | lincRNA |
| AC015849.16 | DFCI-5243 | 17 | 34235658 | 34235658 | lincRNA |
| AHNAK | DFCI-5243 | 11 | 62298814 | 62298814 | Silent |
| ANPEP | DFCI-5243 | 15 | 90346930 | 90346930 | Missense_Mutation |
| ATP4A | DFCI-5243 | 19 | 36051399 | 36051399 | Missense_Mutation |
| B3GALT2 | DFCI-5243 | 1 | 193150535 | 193150535 | Missense_Mutation |
| BHLHE40 | DFCI-5243 | 3 | 5021888 | 5021888 | Intron |
| BUB1 | DFCI-5243 | 2 | 111413397 | 111413397 | Missense_Mutation |
| C17orf70 | DFCI-5243 | 17 | 79517349 | 79517349 | Missense_Mutation |
| C1orf101 | DFCI-5243 | 1 | 244715837 | 244715837 | Silent |
| C1orf141 | DFCI-5243 | 1 | 67561036 | 67561036 | Missense_Mutation |
| C8orf37 | DFCI-5243 | 8 | 96264494 | 96264494 | Missense_Mutation |
| CEP152 | DFCI-5243 | 15 | 49085631 | 49085631 | Missense_Mutation |
| CHD2 | DFCI-5243 | 15 | 93557977 | 93557977 | Nonsense_Mutation |
| CHRNB2 | DFCI-5243 | 1 | 154542795 | 154542795 | Missense_Mutation |
| COL12A1 | DFCI-5243 | 6 | 75818846 | 75818846 | Missense_Mutation |
| CPZ | DFCI-5243 | 4 | 8620213 | 8620213 | Missense_Mutation |
| CRIM1 | DFCI-5243 | 2 | 36739471 | 36739471 | Missense_Mutation |
| CRIP2 | DFCI-5243 | 14 | 105944697 | 105944697 | Splice_Site |
| DKK3 | DFCI-5243 | 11 | 11989955 | 11989955 | Missense_Mutation |
| DNAH8 | DFCI-5243 | 6 | 38819441 | 38819441 | Silent |
| DOCK1 | DFCI-5243 | 10 | 129249683 | 129249683 | Missense_Mutation |
| FAR2 | DFCI-5243 | 12 | 29446197 | 29446197 | Intron |
| FLNB | DFCI-5243 | 3 | 58132626 | 58132626 | Silent |
| FTMT | DFCI-5243 | 5 | 121187843 | 121187843 | Missense_Mutation |
| GAL3ST2 | DFCI-5243 | 2 | 242742809 | 242742809 | Missense_Mutation |
| GIT1 | DFCI-5243 | 17 | 27902682 | 27902682 | Missense_Mutation |
| GUCA2A | DFCI-5243 | 1 | 42628585 | 42628585 | Missense_Mutation |
| IGHG1 | DFCI-5243 | 14 | 106208271 | 106208271 | RNA |
| IGHJ6 | DFCI-5243 | 14 | 106330112 | 106330112 | RNA |
| IGHJ6 | DFCI-5243 | 14 | 106330303 | 106330303 | RNA |
| IGHM | DFCI-5243 | 14 | 106322169 | 106322169 | RNA |
| IGHV2-26 | DFCI-5243 | 14 | 106757764 | 106757764 | RNA |
| IGHV3-23 | DFCI-5243 | 14 | 106725322 | 106725322 | RNA |
| IGHV3-23 | DFCI-5243 | 14 | 106725401 | 106725401 | RNA |
| IGHV3-23 | DFCI-5243 | 14 | 106725638 | 106725638 | RNA |
| IGLV3-21 | DFCI-5243 | 22 | 23055439 | 23055439 | RNA |
| IGLV3-21 | DFCI-5243 | 22 | 23055509 | 23055509 | RNA |
| IGLV3-21 | DFCI-5243 | 22 | 23055535 | 23055535 | RNA |
| IGLV4-3 | DFCI-5243 | 22 | 23213939 | 23213939 | RNA |
| IGLV4-3 | DFCI-5243 | 22 | 23213951 | 23213951 | RNA |
| IGLV4-3 | DFCI-5243 | 22 | 23214029 | 23214029 | RNA |
| IGLV4-3 | DFCI-5243 | 22 | 23214032 | 23214032 | RNA |
| IGLV4-3 | DFCI-5243 | 22 | 23214070 | 23214070 | RNA |
| LAMB4 | DECI-5243 | 7 | 107748104 | 107748104 | Missense_Mutation |
| LPPR4 | DFCI-5243 | 1 | 99771478 | 99771478 | Missense_Mutation |
| LRP1 | DFCI-5243 | 12 | 57593084 | 57593084 | Nonsense_Mutation |
| MEFV | DFCI-5243 | 16 | 3304458 | 3304458 | Intron |
| MLH3 | DFCI-5243 | 14 | 75515428 | 75515428 | Nonsense_Mutation |
| MMP16 | DFCI-5243 | 8 | 89053931 | 89053931 | Missense_Mutation |
| MYH7 | DFCI-5243 | 14 | 23884924 | 23884924 | Missense_Mutation |
| NAALADL2 | DFCI-5243 | 3 | 174814707 | 174814707 | Silent |
| NLRC5 | DFCI-5243 | 16 | 57060669 | 57060669 | Missense_Mutation |
| NPAT | DFCI-5243 | 11 | 108031824 | 108031824 | Missense_Mutation |
| NRXN3 | DFCI-5243 | 14 | 80328170 | 80328170 | Missense_Mutation |
| NUAK1 | DFCI-5243 | 12 | 106477645 | 106477645 | Silent |
| OR4K14 | DFCI-5243 | 14 | 20482830 | 20482830 | Missense_Mutation |
| PAQR4 | DFCI-5243 | 16 | 3021331 | 3021331 | Intron |
| PCLO | DFCI-5243 | 7 | 82582228 | 82582228 | Missense_Mutation |
| PLA2G2E | DFCI-5243 | 1 | 20246902 | 20246902 | Missense_Mutation |
| PLCB1 | DFCI-5243 | 20 | 8665676 | 8665676 | Missense_Mutation |
| PPA1 | DFCI-5243 | 10 | 71977629 | 71977629 | Missense_Mutation |
| RAPGEF2 | DFCI-5243 | 4 | 160279443 | 160279443 | 3′UTR |
| RCC2 | DFCI-5243 | 1 | 17752073 | 17752073 | Missense_Mutation |
| REEP6 | DFCI-5243 | 19 | 1497371 | 1497371 | 3′UTR |
| RGMA | DFCI-5243 | 15 | 93595250 | 93595250 | Silent |
| RING1 | DFCI-5243 | 6 | 33177736 | 33177736 | Missense_Mutation |
| RNF213 | DFCI-5243 | 17 | 78313787 | 78313787 | Missense_Mutation |
| SALL1 | DFCI-5243 | 16 | 51174769 | 51174769 | 5′Flank |
| SCNN1A | DFCI-5243 | 12 | 6464558 | 6464558 | Silent |
| SLC35E2B | DFCI-5243 | 1 | 1602985 | 1602985 | Missense_Mutation |
| SNAP91 | DFCI-5243 | 6 | 84300969 | 84300969 | Missense_Mutation |
| SNAP91 | DFCI-5243 | 6 | 84417596 | 84417596 | Silent |
| SNCAIP | DFCI-5243 | 5 | 121776408 | 121776408 | Missense_Mutation |
| SPDYE4 | DFCI-5243 | 17 | 8660637 | 8660637 | Nonsense_Mutation |
| SYMPK | DFCI-5243 | 19 | 46351042 | 46351042 | Missense_Mutation |
| TENM3 | DFCI-5243 | 4 | 183674641 | 183674641 | Missense_Mutation |
| TIMD4 | DFCI-5243 | 5 | 156381444 | 156381444 | Missense_Mutation |
| TMEM178A | DFCI-5243 | 2 | 39931271 | 39931271 | Missense_Mutation |
| TMEM216 | DFCI-5243 | 11 | 61160711 | 61160711 | Missense_Mutation |
| TMEM242 | DFCI-5243 | 6 | 157744517 | 157744517 | Silent |
| TMPRSS9 | DFCI-5243 | 19 | 2399142 | 2399142 | Silent |
| TRIM27 | DFCI-5243 | 6 | 28879492 | 28879492 | Missense_Mutation |
| TRIML2 | DFCI-5243 | 4 | 189019400 | 189019400 | Nonsense_Mutation |
| UGT3A1 | DFCI-5243 | 5 | 35965535 | 35965535 | Missense_Mutation |
| WDR46 | DFCI-5243 | 6 | 33256726 | 33256726 | Missense_Mutation |
| ZFP36L2 | DFCI-5243 | 2 | 43452046 | 43452046 | Silent |
| ZNF488 | DFCI-5243 | 10 | 48370750 | 48370750 | Missense_Mutation |
| SF3B1 | DFCI-5243 | 2 | 198267364 | 198267364 | Missense_Mutation |
| SF3B1 | DFCI-5243 | 2 | 198267360 | 198267360 | Missense_Mutation |
| SF3B1 | DFCI-5243 | 2 | 198267355 | 198267355 | Missense_Mutation |
| TP53 | DFCI-5243 | 17 | 7577568 | 7577568 | Missense_Mutation |
| gain_8q24.3 | DFCI-5243 | 8 | 0 | 0 | focal_level |
| ADNP2 | DFCI-5243 | 18 | 77896576 | 77896576 | Frame_Shift_Del |
| loss_9p21.2 | DFCI-5243 | 9 | 0 | 0 | focal_level |
| loss_9p21.3 | DFCI-5243 | 9 | 0 | 0 | focal_level |
| SEC16A | DFCI-5243 | 9 | 139354108 | 139354108 | Intron |
| homdel_14q32.33 | DFCI-5243 | 14 | 106436541 | 106436541 | focal_level |
| loss_18p | DFCI-5243 | 18 | 0 | 0 | arm_level |
| gain_MYC | DFCI-5243 | 8 | 0 | 0 | focal_level |
| loss_9q34.3 | DFCI-5243 | 9 | 0 | 0 | focal_level |
| gain_1q23.3 | DFCI-5243 | 1 | 0 | 0 | focal_level |
| gain_22q11.21 | DFCI-5243 | 22 | 0 | 0 | focal_level |
| loss_5p | DFCI-5243 | 5 | 0 | 0 | arm_level |
| loss_14q | DFCI-5243 | 14 | 0 | 0 | arm_level |
| loss_5q | DFCI-5243 | 5 | 0 | 0 | arm_level |
| C5orf45 | DFCI-5243 | 5 | 179278379 | 179278379 | Intron |
| DNAH6 | DFCI-5243 | 2 | 84851685 | 84851685 | Frame_Shift_Del |
| loss_8p11.21 | DFCI-5243 | 8 | 0 | 0 | focal_level |
| gain_4q | DFCI-5243 | 4 | 0 | 0 | arm_level |
| gain_4p | DFCI-5243 | 4 | 0 | 0 | arm_level |
| loss_18q23 | DFCI-5243 | 18 | 0 | 0 | focal_level |
| loss_9p24.1 | DFCI-5243 | 9 | 0 | 0 | focal_level |
| ZFC3H1 | DFCI-5243 | 12 | 72004352 | 72004352 | Intron |
| loss_PARK2 | DFCI-5243 | 6 | 0 | 0 | focal_level |
| gain_MCL1 | DFCI-5243 | 1 | 0 | 0 | focal_level |
| LIFR | DFCI-5243 | 5 | 38506742 | 38506742 | Intron |
| loss_4q | DFCI-5243 | 4 | 0 | 0 | arm_level |
| gain_8q | DFCI-5243 | 8 | 91813930 | 91813930 | arm_level |
| gain_8p | DFCI-5243 | 8 | 0 | 0 | arm_level |
| loss_4p | DFCI-5243 | 4 | 0 | 0 | arm_level |
| gain_10q | DFCI-5243 | 10 | 0 | 0 | arm_level |
| gain_18p | DFCI-5243 | 18 | 0 | 0 | arm_level |
| PDE6A | DFCI-5243 | 5 | 149247333 | 149247333 | Frame_Shift_Del |
| C10orf137 | DFCI-5243 | 10 | 127422796 | 127422796 | Intron |
| gain_8q23.3 | DECI-5243 | 8 | 0 | 0 | focal_level |
| loss_RB1 | DFCI-5243 | 13 | 0 | 0 | focal_level |
| loss_10q | DFCI-5243 | 10 | 0 | 0 | arm_level |
| gain_8q22.2 | DFCI-5243 | 8 | 0 | 0 | focal_level |
| loss_8p | DFCI-5243 | 8 | 0 | 0 | arm_level |
| RAB4B-EGLN2 | DFCI-5243 | 19 | 41286513 | 41286513 | Intron |
| gain_13q | DFCI-5243 | 13 | 0 | 0 | arm_level |
| CHD2 | DFCI-5243 | 15 | 93489277 | 93489277 | Frame_Shift_Del |
| DDX11 | DFCI-5243 | 12 | 31255035 | 31255035 | Intron |
| gain_7q36.3 | DFCI-5243 | 7 | 0 | 0 | focal_level |
| MYB | DFCI-5243 | 6 | 135507082 | 135507082 | Frame_Shift_Del |
| homdel_RB1 | DFCI-5243 | 13 | 46946342 | 46946342 | focal_level |
| gain_6q | DFCI-5243 | 6 | 0 | 0 | arm_level |
| SRSF12 | DFCI-5243 | 6 | 89816808 | 89816808 | Intron |
| gain_7q | DFCI-5243 | 7 | 0 | 0 | arm_level |
| gain_7p | DFCI-5243 | 7 | 0 | 0 | arm_level |
| loss_6p | DFCI-5243 | 6 | 0 | 0 | arm_level |
| loss_17p13.3 | DFCI-5243 | 17 | 11268 | 11268 | focal_level |
| loss_17p | DFCI-5243 | 17 | 0 | 0 | arm_level |
| loss_10p15.3 | DFCI-5243 | 10 | 0 | 0 | focal_level |
| gain_6p | DFCI-5243 | 6 | 0 | 0 | arm_level |
| gain_5q | DFCI-5243 | 5 | 0 | 0 | arm_level |
| loss_21q | DFCI-5243 | 21 | 0 | 0 | arm_level |
| MTR | DFCI-5243 | 1 | 237013559 | 237013559 | Intron |
| ANGPTL6 | DFCI-5591 | 19 | 10204247 | 10204247 | Missense_Mutation |
| BCAR1 | DFCI-5591 | 16 | 75271198 | 75271198 | Silent |
| BHLHA15 | DFCI-5591 | 7 | 97841917 | 97841917 | Missense_Mutation |
| BID | DFCI-5591 | 22 | 18218233 | 18218233 | 3′UTR |
| C10orf11 | DFCI-5591 | 10 | 77818427 | 77818427 | Nonsense_Mutation |
| C7 | DFCI-5591 | 5 | 40972658 | 40972658 | Nonsense_Mutation |
| CEND1 | DFCI-5591 | 11 | 788346 | 788346 | Missense_Mutation |
| CXCR2 | DFCI-5591 | 2 | 219000146 | 219000146 | Missense_Mutation |
| DEFB114 | DFCI-5591 | 6 | 49928146 | 49928146 | Silent |
| DNAH5 | DFCI-5591 | 5 | 13810295 | 13810295 | Silent |
| DNHD1 | DFCI-5591 | 11 | 6561128 | 6561128 | Missense_Mutation |
| EDNRA | DFCI-5591 | 4 | 148406926 | 148406926 | Silent |
| EXOC6B | DFCI-5591 | 2 | 72727078 | 72727078 | Silent |
| GRM5 | DFCI-5591 | 11 | 88323800 | 88323800 | Silent |
| HADH | DFCI-5591 | 4 | 108926012 | 108926012 | Intron |
| IRF8 | DFCI-5591 | 16 | 85952268 | 85952268 | Missense_Mutation |
| KIF20B | DFCI-5591 | 10 | 91528048 | 91528048 | Splice_Site |
| LPHN3 | DFCI-5591 | 4 | 62936529 | 62936529 | Missense_Mutation |
| MPP5 | DFCI-5591 | 14 | 67768145 | 67768145 | Nonsense_Mutation |
| MT1A | DFCI-5591 | 16 | 56669949 | 56669949 | 5′Flank |
| MTIF3 | DFCI-5591 | 13 | 28009715 | 28009715 | IGR |
| NAV2 | DFCI-5591 | 11 | 20044578 | 20044578 | Intron |
| NCKAP5 | DFCI-5591 | 2 | 133543162 | 133543162 | Silent |
| NDRG1 | DFCI-5591 | 8 | 134260968 | 134260968 | Missense_Mutation |
| PDZRN4 | DFCI-5591 | 12 | 41966165 | 41966165 | Splice_Site |
| RBM38 | DFCI-5591 | 20 | 55967758 | 55967758 | Missense_Mutation |
| RD3 | DFCI-5591 | 1 | 211654657 | 211654657 | Missense_Mutation |
| RIOK1 | DFCI-5591 | 6 | 7403005 | 7403005 | Intron |
| ROBO4 | DFCI-5591 | 11 | 124756289 | 124756289 | Intron |
| SAMHD1 | DFCI-5591 | 20 | 35545453 | 35545453 | Splice_Site |
| SAMHD1 | DFCI-5591 | 20 | 35545450 | 35545450 | Nonsense_Mutation |
| SEMA4G | DFCI-5591 | 10 | 102738735 | 102738735 | Missense_Mutation |
| SEMA6D | DFCI-5591 | 15 | 48062908 | 48062908 | Silent |
| SLC25A10 | DFCI-5591 | 17 | 79682553 | 79682553 | Missense_Mutation |
| SPATS2 | DFCI-5591 | 12 | 49893882 | 49893882 | Missense_Mutation |
| TENM2 | DFCI-5591 | 5 | 167379775 | 167379775 | Missense_Mutation |
| TMEM176B | DFCI-5591 | 7 | 150487604 | 150487604 | IGR |
| TMPRSS15 | DFCI-5591 | 21 | 19666585 | 19666585 | Splice_Site |
| TNIP3 | DFCI-5591 | 4 | 122085425 | 122085425 | Intron |
| TRAF5 | DFCI-5591 | 1 | 211526705 | 211526705 | Missense_Mutation |
| TRBC2 | DFCI-5591 | 7 | 142498969 | 142498969 | RNA |
| ZNF16 | DFCI-5591 | 8 | 146157523 | 146157523 | Missense_Mutation |
| ZNF292 | DFCI-5591 | 6 | 87964825 | 87964825 | Missense_Mutation |
| ZNF296 | DFCI-5591 | 19 | 45575370 | 45575370 | Missense_Mutation |
| ZNF629 | DFCI-5591 | 16 | 30793826 | 30793826 | Missense_Mutation |
| FAT1 | DFCI-5591 | 4 | 187628840 | 187628840 | Silent |
| KEL | DFCI-5591 | 7 | 142658532 | 142658532 | Silent |
| NRAS | DFCI-5591 | 1 | 115256529 | 115256529 | Missense_Mutation |
| loss_14q32.33 | DFCI-5591 | 14 | 106478155 | 106478155 | focal_level |
| SAMHD1 | DFCI-5591 | 20 | 35545235 | 35545235 | Splice_Site |
| IRF8 | DFCI-5591 | 16 | 85954853 | 85954853 | Frame_Shift_Del |
| homdel_APC | DFCI-5591 | 5 | 89854675 | 89854675 | focal_level |
| homdel_14q32.33 | DFCI-5591 | 14 | 0 | 0 | focal_level |
| HAL | DFCI-5591 | 12 | 96379985 | 96379985 | Intron |
| NOTCH1 | DFCI-5591 | 9 | 139390649 | 139390649 | Frame_Shift_Del |
| XPO4 | DFCI-5591 | 13 | 21370223 | 21370223 | Frame_Shift_Del |
| SAMHD1 | DFCI-5591 | 20 | 35526886 | 35526886 | Frame_Shift_Ins |
| RAI1 | DFCI-5591 | 17 | 17699398 | 17699398 | Frame_Shift_Ins |
| ABCA13 | JB-0126 | 7 | 48314267 | 48314267 | Silent |
| ANO5 | JB-0126 | 11 | 22283745 | 22283745 | Missense_Mutation |
| ASXL3 | JB-0126 | 18 | 31318986 | 31318986 | Missense_Mutation |
| ATP12A | JB-0126 | 13 | 25283966 | 25283966 | Splice_Site |
| BRSK2 | JB-0126 | 11 | 1466575 | 1466575 | Missense_Mutation |
| C17orf62 | JB-0126 | 17 | 80401837 | 80401837 | Intron |
| CDK5R1 | JB-0126 | 17 | 30815159 | 30815159 | Missense_Mutation |
| COL12A1 | JB-0126 | 6 | 75851747 | 75851747 | Splice_Site |
| COX15 | JB-0126 | 10 | 101476107 | 101476107 | Nonsense_Mutation |
| CTD-3064M3.7 | JB-0126 | 8 | 142446906 | 142446906 | RNA |
| DROSHA | JB-0126 | 5 | 31422948 | 31422948 | Missense_Mutation |
| DSEL | JB-0126 | 18 | 65180361 | 65180361 | Missense_Mutation |
| FAM120A | JB-0126 | 9 | 96318716 | 96318716 | Missense_Mutation |
| FSIP2 | JB-0126 | 2 | 186673166 | 186673166 | Nonsense_Mutation |
| GALNT5 | JB-0126 | 2 | 158156101 | 158156101 | IGR |
| GPT | JB-0126 | 8 | 145731356 | 145731356 | Intron |
| GRIK3 | JB-0126 | 1 | 37267592 | 37267592 | Missense_Mutation |
| IRAK1BP1 | JB-0126 | 6 | 79607875 | 79607875 | Missense_Mutation |
| MIR518C | JB-0126 | 19 | 54209520 | 54209520 | RNA |
| MYH9 | JB-0126 | 22 | 36689375 | 36689375 | Splice_Site |
| NXF1 | JB-0126 | 11 | 62571282 | 62571282 | Missense_Mutation |
| OR5AR1 | JB-0126 | 11 | 56431642 | 56431642 | Missense_Mutation |
| OR5J2 | JB-0126 | 11 | 55944294 | 55944294 | Silent |
| PDIA3 | JB-0126 | 15 | 44062481 | 44062481 | Missense_Mutation |
| PGLYRP3 | JB-0126 | 1 | 153283186 | 153283186 | 5′UTR |
| RCC1 | JB-0126 | 1 | 28835165 | 28835165 | Intron |
| RP11-343C2.12 | JB-0126 | 16 | 69362870 | 69362870 | 3′UTR |
| RPL22L1 | JB-0126 | 3 | 170585988 | 170585988 | Intron |
| SLC12A5 | JB-0126 | 20 | 44675042 | 44675042 | Missense_Mutation |
| SNHG14 | JB-0126 | 15 | 25328564 | 25328564 | RNA |
| TENM3 | JB-0126 | 4 | 183600992 | 183600992 | Missense_Mutation |
| TOM1L2 | JB-0126 | 17 | 17787978 | 17787978 | Silent |
| TP53 | JB-0126 | 17 | 7577498 | 7577498 | Splice_Site |
| TP53 | JB-0126 | 17 | 7577610 | 7577610 | Splice_Site |
| TRIM37 | JB-0126 | 17 | 57125047 | 57125047 | Missense_Mutation |
| TRIP4 | JB-0126 | 15 | 64698567 | 64698567 | Missense_Mutation |
| TUBGCP2 | JB-0126 | 10 | 135111566 | 135111566 | Missense_Mutation |
| ZNF12 | JB-0126 | 7 | 6746108 | 6746108 | 5′UTR |
| ZNF254 | JB-0126 | 19 | 24216365 | 24216365 | 5′UTR |
| ZNF560 | JB-0126 | 19 | 9577562 | 9577562 | Silent |
| loss_14q32.33 | JB-0126 | 14 | 106757700 | 106757700 | focal_level |
| PTPN11 | JB-0126 | 12 | 112888166 | 112888166 | Missense_Mutation |
| SF3B1 | JB-0126 | 2 | 198266611 | 198266611 | Splice_Site |
| SF3B1 | JB-0126 | 2 | 198267360 | 198267360 | Missense_Mutation |
| TP53 | JB-0126 | 17 | 7578395 | 7578395 | Missense_Mutation |
| TP53 | JB-0126 | 17 | 7578538 | 7578538 | Missense_Mutation |
| TP53 | JB-0126 | 17 | 7577563 | 7577563 | Missense_Mutation |
| homdel_RB1 | JB-0126 | 13 | 0 | 0 | focal_level |
| TC2N | JB-0126 | 14 | 92251797 | 92251797 | Intron |
| gain_22q11.21 | JB-0126 | 22 | 0 | 0 | focal_level |
| loss_11p15.5 | JB-0126 | 11 | 619532 | 619532 | focal_level |
| SYNE1 | JB-0126 | 6 | 152763448 | 152763448 | Intron |
| homdel_APC | JB-0126 | 5 | 0 | 0 | focal_level |
| BAZ2A | JB-0126 | 12 | 56994757 | 56994757 | Frame_Shift_Del |

| Hugo_Symbol | Variant_Type | Reference_Allele | Tumor_Seq_Allele1 | Tumor_Seq_Allele2 |
|---|---|---|---|---|
| Unknown | SNP | C | C | T |
| AATK | SNP | G | G | A |
| ABCD4 | SNP | G | G | C |
| ABCF1 | SNP | G | G | A |
| ABTB2 | SNP | C | C | T |
| ACAD10 | SNP | G | G | T |
| ACAT1 | SNP | T | T | C |
| ACD | SNP | T | T | G |
| ACIN1 | SNP | T | T | C |
| ACKR3 | SNP | C | C | T |
| ADC | SNP | C | C | G |
| ADORA3 | SNP | C | C | T |
| AHNAK2 | SNP | G | G | A |
| ALG8 | SNP | G | G | A |
| ANO9 | SNP | A | A | G |
| ARMC12 | SNP | C | C | T |
| ARSK | SNP | G | G | A |
| ASAP2 | SNP | G | G | A |
| ATP13A1 | SNP | G | G | A |
| ATP6V0B | SNP | G | G | A |
| ATP6V0B | SNP | G | G | A |
| ATP8B4 | SNP | T | T | c |
| BFSP1 | SNP | G | G | T |
| C15orf27 | SNP | C | C | G |
| C15orf39 | SNP | G | G | A |
| C16orf45 | SNP | A | A | C |
| C19orf18 | SNP | G | G | A |
| C21orf58 | SNP | C | C | T |
| C2CD2 | SNP | C | C | T |
| CACNA1S | SNP | C | C | G |
| CAD | SNP | G | G | A |
| CADM3 | SNP | C | C | T |
| CAPRIN2 | SNP | G | G | A |
| CASP5 | SNP | T | T | G |
| CCDC57 | SNP | G | G | A |
| CDC42BPB | SNP | C | C | T |
| CDK6 | SNP | G | G | T |
| CENPT | SNP | C | C | T |
| CEPT1 | SNP | T | T | G |
| CES4A | SNP | G | G | A |
| CFTR | SNP | A | A | T |
| CHRNA4 | SNP | C | C | A |
| CHRNA4 | SNP | C | C | A |
| CHRNB3 | SNP | A | A | G |
| CHTF18 | SNP | A | A | G |
| CLTB | SNP | G | G | A |
| COL18A1 | SNP | C | C | G |
| COMMD4 | SNP | C | C | G |
| CSMD2 | SNP | C | C | T |
| CTBP1 | SNP | C | C | T |
| CYP1A2 | SNP | G | G | T |
| DEFA4 | SNP | A | A | T |
| DEFB1 | SNP | T | T | C |
| DIABLO | SNP | A | A | T |
| DOPEY2 | SNP | C | C | G |
| DST | SNP | T | T | C |
| DYNC1H1 | SNP | A | A | G |
| DYSF | SNP | C | C | G |
| EIF2AK4 | SNP | T | T | C |
| ENPEP | SNP | G | G | A |
| EP300 | SNP | T | T | C |
| EPHB4 | SNP | C | C | T |
| ERCC6 | SNP | G | G | A |
| ESYT1 | SNP | G | G | A |
| EXPH5 | SNP | C | C | G |
| EXPH5 | SNP | G | G | T |
| FAM111B | SNP | C | C | T |
| FAM65B | SNP | C | C | G |
| FAM71B | SNP | T | T | G |
| FBXL16 | SNP | G | G | A |
| FLNB | SNP | G | G | A |
| FLYWCH2 | SNP | G | G | A |
| FNBP4 | SNP | C | C | T |
| FNBP4 | SNP | G | G | C |
| FZD8 | SNP | C | C | G |
| GCC1 | SNP | G | G | A |
| GNA15 | SNP | C | C | T |
| GPR144 | SNP | C | C | T |
| GRIP1 | SNP | C | C | T |
| HAVCR1 | SNP | G | G | A |
| HGD | SNP | C | C | T |
| HNRNPM | SNP | C | C | T |
| HNRNPUL2 | SNP | T | T | C |
| HSD17B12 | SNP | G | G | A |
| HSDL2 | SNP | T | T | C |
| IGHM | SNP | C | C | A |
| INTS6 | SNP | A | A | G |
| IQGAP1 | SNP | A | A | G |
| ITPR1 | SNP | G | G | A |
| JMJD1C | SNP | G | G | A |
| JMJD1C | SNP | G | G | T |
| JPH3 | SNP | G | G | A |
| JPH4 | SNP | G | G | A |
| KDELR2 | SNP | G | G | A |
| KIAA0020 | SNP | T | T | C |
| KIAA1109 | SNP | A | A | G |
| KLF11 | SNP | A | A | G |
| KNTC1 | SNP | C | C | A |
| KRT35 | SNP | C | C | A |
| KRTAP24-1 | SNP | T | T | A |
| LINC00207 | SNP | T | T | C |
| LRIG2 | SNP | A | A | G |
| LRRC16B | SNP | C | C | A |
| LSM14B | SNP | G | G | A |
| MB21D2 | SNP | T | T | C |
| MCF2L2 | SNP | C | C | A |
| MED12L | SNP | G | G | A |
| MIR1207 | SNP | A | A | G |
| MLXIP | SNP | T | T | C |
| MSLNL | SNP | G | G | A |
| MST1 | SNP | A | A | G |
| MT1L | SNP | T | T | C |
| MT3 | SNP | G | G | A |
| MTHFD2L | SNP | A | A | G |
| MUC17 | SNP | G | G | A |
| MYO10 | SNP | G | G | C |
| NARS | SNP | T | T | G |
| NCBP1 | SNP | G | G | T |
| NCOR2 | SNP | C | C | T |
| NDUFB10 | SNP | T | T | C |
| NEK9 | SNP | A | A | G |
| NEU4 | SNP | C | C | A |
| NFATC4 | SNP | G | G | A |
| NKX2-6 | SNP | A | A | G |
| NNT | SNP | C | C | T |
| NPAT | SNP | G | G | C |
| NRXN1 | SNP | G | G | A |
| NUMBL | SNP | G | G | T |
| OGFOD2 | SNP | C | C | T |
| ORAI3 | SNP | G | G | A |
| OTUD7B | SNP | A | A | G |
| OXSR1 | SNP | C | C | A |
| PAFAH1B3 | SNP | C | C | G |
| PALD1 | SNP | A | A | G |
| PAPPA | SNP | T | T | C |
| PCDHGB3 | SNP | G | G | A |
| PCID2 | SNP | T | T | C |
| PDE6A | SNP | G | G | A |
| PI4KA | SNP | G | G | A |
| PIK3C2A | SNP | C | C | T |
| PKD1L1 | SNP | G | G | T |
| PKD1L3 | SNP | A | A | G |
| PKD2L2 | SNP | C | C | A |
| PMEL | SNP | C | C | T |
| POU5F1 | SNP | C | C | A |
| PPM1D | SNP | C | C | G |
| PPP1R1B | SNP | G | G | C |
| PPP4R1 | SNP | T | T | C |
| PRKD3 | SNP | A | A | C |
| PRR24 | SNP | C | C | T |
| PSD4 | SNP | C | C | T |
| RAB3GAP2 | SNP | G | G | A |
| RAC2 | SNP | G | G | A |
| RAPGEF6 | SNP | C | C | T |
| RASGRF1 | SNP | C | C | A |
| RP11-54D18.3 | SNP | T | T | C |
| RP11-794P6.2 | SNP | C | C | A |
| RTKN | SNP | C | C | T |
| RTN3 | SNP | C | C | A |
| SDCCAG8 | SNP | C | C | T |
| SH3BGR | SNP | C | C | A |
| SLC13A3 | SNP | C | C | T |
| SLC17A4 | SNP | C | C | T |
| SLC25A15 | SNP | C | C | T |
| SLIRP | SNP | G | G | A |
| SON | SNP | G | G | A |
| SORCS2 | SNP | C | C | G |
| SPAG6 | SNP | T | T | C |
| SRRM2 | SNP | A | A | T |
| SUSD2 | SNP | C | C | T |
| SYT14 | SNP | T | T | C |
| SZT2 | SNP | G | G | A |
| TDRD6 | SNP | A | A | G |
| TDRD6 | SNP | C | C | A |
| THBS1 | SNP | G | G | A |
| TIE1 | SNP | A | A | G |
| TIE1 | SNP | G | G | A |
| TLR4 | SNP | G | G | A |
| TMED4 | SNP | A | A | T |
| TNFAIP8 | SNP | C | C | T |
| TNIK | SNP | G | G | A |
| TRPC1 | SNP | G | G | A |
| TSPAN4 | SNP | C | C | T |
| UBA7 | SNP | A | A | G |
| USP37 | SNP | T | T | C |
| USP4 | SNP | G | G | A |
| VPS4A | SNP | C | C | T |
| VWA8 | SNP | T | T | G |
| WDR78 | SNP | G | G | C |
| WIPF1 | SNP | G | G | A |
| YPEL4 | SNP | C | C | T |
| ZCCHC4 | SNP | A | A | G |
| ZFHX4 | SNP | A | A | G |
| ZMYM6 | SNP | C | C | T |
| ZNF215 | SNP | A | A | G |
| ZNF219 | SNP | G | G | C |
| ZNF28 | SNP | C | C | T |
| ZNF408 | SNP | C | C | T |
| ZNF408 | SNP | G | G | T |
| ZNF408 | SNP | G | G | T |
| ZNF517 | SNP | A | A | G |
| ZNF560 | SNP | C | C | T |
| ZNF696 | SNP | C | C | T |
| ZNF883 | SNP | C | C | A |
| loss_17p | CNV | None | None | None |
| DIS3 | SNP | C | C | A |
| FLG | SNP | C | C | A |
| MTOR | SNP | T | T | C |
| DYSF | DEL | C | C | — |
| C1orf141 | DEL | TTGTCT | TTGTCT | — |
| NFKBIA | DEL | TTTTC | TTTTC | — |
| COL1A1 | DEL | G | G | — |
| SIPA1 | DEL | G | G | — |
| DPAGT1 | DEL | A | A | — |
| PRPSAP1 | DEL | ACTCT | ACTCT | — |
| FN3K | DEL | GAGT | GAGT | — |
| MGP | INS | — | — | T |
| GUCY2D | DEL | CTG | CTG | — |
| LMBR1 | DEL | TATC | TATC | — |
| COL6A1 | DEL | T | T | — |
| TRPM4 | DEL | G | G | — |
| RP11-295K3.1 | DEL | CCAGCC | CCAGCC | — |
| AE000661.50 | INS | — | — | A |
| C17orf66 | INS | — | — | A |
| CLEC17A | DEL | C | C | — |
| EXOSC9 | DEL | CA | CA | — |
| FARP2 | INS | — | — | A |
| SON | DEL | AGGTTAGCTCA | AGGTTAGCTCA | — |
| | | GGATCCTTAC | GGATCCTTAC | |
| NUDT17 | DEL | G | G | — |
| AKAP12 | SNP | C | C | G |
| PLEKHG1 | SNP | G | G | A |
| SYNE1 | SNP | G | G | C |
| HORMAD2 | DEL | AC | AC | — |
| ATP8B2 | SNP | C | C | T |
| CTD-2090I13.1 | SNP | C | C | A |
| LYST | SNP | A | A | G |
| EXOC6B | INS | — | — | GG |
| ADAMTS13 | SNP | G | G | A |
| ADRA1A | SNP | G | G | A |
| AGAP1 | SNP | G | G | A |
| ALOX15B | SNP | A | A | G |
| ALOX15B | SNP | T | T | C |
| ASXL3 | SNP | A | A | T |
| BAI3 | SNP | T | T | C |
| BMP2 | SNP | C | C | T |
| C1orf228 | SNP | G | G | A |
| C6orf223 | SNP | C | C | A |
| CDH23 | SNP | C | C | T |
| CHST9 | SNP | C | C | G |
| CHSY3 | SNP | A | A | T |
| CLCN6 | SNP | T | T | A |
| CLCN6 | SNP | G | G | T |
| DMXL1 | SNP | G | G | T |
| EDC3 | SNP | T | T | C |
| FAM160B1 | SNP | T | T | G |
| GIMAP7 | SNP | A | A | T |
| HERC2 | SNP | C | C | T |
| HIST1H2AC | SNP | G | G | A |
| HTR7 | SNP | C | C | A |
| IGSF9B | SNP | G | G | A |
| JUNB | SNP | G | G | A |
| KIRREL | SNP | G | G | A |
| MAGI2 | SNP | C | C | T |
| NLRP5 | SNP | C | C | T |
| OAS2 | SNP | A | A | G |
| PAX9 | SNP | G | G | A |
| PIWIL2 | SNP | C | C | A |
| PSG9 | SNP | G | G | A |
| SEC11C | SNP | G | G | C |
| SEC14L3 | SNP | A | A | G |
| SLC18A3 | SNP | G | G | T |
| SLC26A9 | SNP | G | G | A |
| TMEM91 | SNP | C | C | T |
| USP8 | SNP | C | C | T |
| XRN1 | SNP | C | C | A |
| XRRA1 | SNP | A | A | T |
| ZNF318 | SNP | G | G | A |
| ATM | SNP | C | C | T |
| ATM | SNP | C | C | T |
| SERPINB13 | SNP | G | G | A |
| SF3B1 | SNP | T | T | C |
| TP53 | SNP | G | G | C |
| gain_1q23.3 | CNV | None | None | None |
| gain_TERT | CNV | NA | NA | NA |
| SFSWAP | INS | — | — | CCC |
| loss_10q25.1 | CNV | NA | NA | NA |
| gain_6p21.1 | CNV | NA | NA | NA |
| gain_22q11.21 | CNV | NA | NA | NA |
| gain_MCL1 | CNV | NA | NA | NA |
| gain_9p24.2 | CNV | NA | NA | NA |
| gain_11q13.2 | CNV | NA | NA | NA |
| AMOTL1 | SNP | C | C | T |
| CADPS2 | SNP | T | T | C |
| GAS2L3 | SNP | C | C | T |
| IDO1 | SNP | G | G | A |
| KBTBD11 | SNP | G | G | T |
| NPR2 | SNP | G | G | A |
| OR10K1 | SNP | G | G | T |
| PGBD1 | SNP | A | A | G |
| PLCH1 | SNP | C | C | A |
| PSMD9 | SNP | C | C | T |
| TAPT1 | SNP | C | C | T |
| TP53 | SNP | G | G | A |
| TSC2 | SNP | C | C | T |
| SLC4A4 | DEL | T | T | — |
| BAZ2A | INS | — | — | A |
| gain_KRAS | CNV | None | None | None |
| loss_6q | CNV | None | None | None |
| DRG2 | SNP | C | C | T |
| homdel_2q37.3 | CNV | None | None | None |
| gain_AKT3 | CNV | None | None | None |
| AP000275.65 | INS | — | — | GCCTCTGCCT |
| ASCC3 | SNP | T | T | C |
| CTNNA2 | SNP | A | A | C |
| DST | SNP | C | C | A |
| FAM179B | SNP | A | A | C |
| MTERFD2 | SNP | C | C | T |
| PNLDC1 | SNP | G | G | C |
| RASSF8 | SNP | A | A | G |
| RDH16 | SNP | T | T | G |
| SYNM | SNP | G | G | T |
| TCTN2 | SNP | G | G | T |
| TRBV25-1 | SNP | C | C | A |
| TRIM24 | SNP | A | A | G |
| gain_MDM4 | CNV | NA | NA | NA |
| gain_11q14.1 | CNV | NA | NA | NA |
| loss_9p13.1 | CNV | NA | NA | NA |
| gain_19q13.42 | CNV | NA | NA | NA |
| gain_11q22.2 | CNV | NA | NA | NA |
| gain_CCND1 | CNV | NA | NA | NA |
| gain_8q21.13 | CNV | NA | NA | NA |
| MTMR3 | SNP | A | A | T |
| MYPN | SNP | C | C | T |
| SPIDR | SNP | T | T | G |
| VNN1 | SNP | A | A | C |
| ERCC2 | SNP | G | G | C |
| homdel_19q13.32 | CNV | None | None | None |
| gain_NKX2-1 | CNV | NA | NA | NA |
| homdel_3p26.3 | CNV | NA | NA | NA |
| homdel_11p15.5 | CNV | None | None | None |
| gain_5q35.3 | CNV | NA | NA | NA |
| loss_16q23.1 | CNV | NA | NA | NA |
| gain_10p15.1 | CNV | None | None | None |
| loss_1p36.23 | CNV | NA | NA | NA |
| homdel_STK11 | CNV | None | None | None |
| gain_ZNF217 | CNV | NA | NA | NA |
| homdel_BRCA1 | CNV | NA | NA | NA |
| gain_3q29 | CNV | None | None | None |
| FAM13C | SNP | C | C | T |
| IGHV1-24 | SNP | T | T | C |
| NDUFA9 | SNP | A | A | C |
| PABPC4L | SNP | C | C | T |
| TDRD9 | SNP | A | A | G |
| homdel_16p13.3 | CNV | None | None | None |
| homdel_17q25.3 | CNV | None | None | None |
| loss_9q34.3 | CNV | None | None | None |
| gain_E2F3 | CNV | None | None | None |
| loss_19q | CNV | None | None | None |
| loss_19p | CNV | None | None | None |
| loss_15q | CNV | None | None | None |
| homdel_12q24.33 | CNV | None | None | None |
| gain_17q11.2 | CNV | None | None | None |
| gain_13q34 | CNV | None | None | None |
| gain_13q | CNV | None | None | None |
| loss_18p | CNV | None | None | None |
| loss_1q44 | CNV | None | None | None |
| homdel_4p16.3 | CNV | None | None | None |
| loss_APC | CNV | None | None | None |
| gain_ERBB2 | CNV | None | None | None |
| gain_9p | CNV | None | None | None |
| homdel_10q26.3 | CNV | None | None | None |
| gain_NEDD9 | CNV | None | None | None |
| homdel_21q22.3 | CNV | None | None | None |
| homdel_16q23.1 | CNV | None | None | None |
| homdel_IKZF2 | CNV | None | None | None |
| homdel_1p36.23 | CNV | None | None | None |
| loss_22q | CNV | None | None | None |
| AMOTL2 | SNP | G | G | A |
| ANAPC4 | SNP | C | C | T |
| ANKRD11 | SNP | G | G | A |
| BIRC3 | SNP | G | G | T |
| C8orf48 | SNP | G | G | T |
| CDH11 | SNP | G | G | A |
| COL6A5 | SNP | G | G | A |
| CORO1A | SNP | A | A | G |
| CPEB2 | SNP | G | G | A |
| DIRAS3 | SNP | G | G | A |
| DNAH9 | SNP | T | T | A |
| EGR2 | SNP | C | C | T |
| ELP3 | SNP | G | G | A |
| ENKD1 | SNP | C | C | T |
| ENO2 | SNP | G | G | A |
| FLNB | SNP | A | A | G |
| FPR2 | SNP | T | T | C |
| GABARAPL2 | SNP | G | G | T |
| GPR133 | SNP | C | C | T |
| HCN1 | SNP | C | C | T |
| IGHMBP2 | SNP | G | G | A |
| IKBIP | SNP | C | C | T |
| INTU | SNP | G | G | C |
| IRS2 | SNP | C | C | T |
| KIAA0196 | SNP | G | G | A |
| MED7 | SNP | G | G | C |
| MICAL3 | SNP | G | G | A |
| MTMR12 | SNP | T | T | C |
| OBSL1 | SNP | C | C | A |
| PCGF5 | SNP | C | C | G |
| SCO1 | SNP | G | G | A |
| SLC12A4 | SNP | T | T | G |
| SLC26A8 | SNP | G | G | A |
| SNED1 | SNP | G | G | A |
| SOX5 | SNP | G | G | C |
| SYNE1 | SNP | C | C | G |
| TACR2 | SNP | C | C | T |
| TBX6 | SNP | G | G | A |
| TDRD6 | SNP | G | G | A |
| TMEM175 | SNP | G | G | A |
| UBA52 | SNP | C | C | G |
| ZNF408 | SNP | A | A | G |
| ATM | SNP | T | T | G |
| IRF4 | SNP | C | C | A |
| IRF4 | SNP | T | T | A |
| TP53 | SNP | C | C | T |
| gain_21q | CNV | NA | NA | NA |
| homdel_17q25.3 | CNV | None | None | None |
| homdel_22q13.32 | CNV | None | None | None |
| homdel_19q13.32 | CNV | None | None | None |
| homdel_14q32.33 | CNV | None | None | None |
| loss_18q23 | CNV | None | None | None |
| homdel_17p13.3 | CNV | None | None | None |
| gain_1q23.3 | CNV | None | None | None |
| NLGN1 | DEL | T | T | — |
| gain_22q11.21 | CNV | None | None | None |
| gain_MDM4 | CNV | None | None | None |
| homdel_10q25.1 | CNV | NA | NA | NA |
| homdel_STK11 | CNV | None | None | None |
| loss_12q24.33 | CNV | NA | NA | NA |
| homdel_19q13.43 | CNV | None | None | None |
| homdel_2q37.3 | CNV | NA | NA | NA |
| gain_ZNF217 | CNV | NA | NA | NA |
| gain_E2F3 | CNV | None | None | None |
| loss_19p | CNV | None | None | None |
| homdel_7p22.3 | CNV | None | None | None |
| gain_4p | CNV | None | None | None |
| gain_12q | CNV | None | None | None |
| gain_MDM2 | CNV | None | None | None |
| gain_NEDD9 | CNV | None | None | None |
| loss_5q35.3 | CNV | None | None | None |
| loss_20p | CNV | None | None | None |
| gain_8p | CNV | NA | NA | NA |
| gain_CCND1 | CNV | NA | NA | NA |
| homdel_21q22.3 | CNV | None | None | None |
| homdel_16q23.1 | CNV | NA | NA | NA |
| gain_18q | CNV | NA | NA | NA |
| loss_16p | CNV | None | None | None |
| loss_11q23.1 | CNV | None | None | None |
| NELL1 | DEL | G | G | — |
| loss_22q | CNV | None | None | None |
| gain_17q24.2 | CNV | NA | NA | NA |
| gain_IGF1R | CNV | None | None | None |
| loss_MLL3 | CNV | NA | NA | NA |
| homdel_1p36.23 | CNV | None | None | None |
| gain_NKX2-1 | CNV | NA | NA | NA |
| gain_13q | CNV | NA | NA | NA |
| loss_15q15.1 | CNV | NA | NA | NA |
| gain_CDK4 | CNV | None | None | None |
| loss_8p21.2 | CNV | NA | NA | NA |
| gain_10p15.1 | CNV | NA | NA | NA |
| gain_11q14.1 | CNV | NA | NA | NA |
| gain_7q36.3 | CNV | None | None | None |
| gain_12p | CNV | None | None | None |
| homdel_10q26.3 | CNV | None | None | None |
| gain_12p13.33 | CNV | None | None | None |
| gain_9p24.2 | CNV | NA | NA | NA |
| homdel_9q34.3 | CNV | None | None | None |
| homdel_4p16.3 | CNV | None | None | None |
| gain_KRAS | CNV | None | None | None |
| homdel_5q35.3 | CNV | NA | NA | NA |
| gain_3q | CNV | None | None | None |
| loss_17p | CNV | None | None | None |
| loss_4p | CNV | None | None | None |
| gain_5q | CNV | None | None | None |
| gain_11q13.2 | CNV | NA | NA | NA |
| homdel_15q15.1 | CNV | None | None | None |
| AGXT2 | SNR | C | C | A |
| BCL11B | SNP | G | G | A |
| BTBD19 | SNR | G | G | A |
| CAPRIN2 | SNP | G | G | A |
| CDH2 | SNP | C | C | T |
| CEP112 | SNP | G | G | A |
| CPN1 | SNP | G | G | A |
| DDX49 | SNP | G | G | C |
| DNAH17 | SNP | C | C | T |
| DTNA | SNP | C | C | T |
| HNF1B | SNP | C | C | T |
| HNF4A | SNP | A | A | C |
| HTRA3 | SNP | G | G | T |
| IL1R2 | SNP | C | C | T |
| ITGA4 | SNP | A | A | T |
| LAPTM5 | SNP | G | G | A |
| MAN1B1 | SNP | G | G | C |
| NBPF22P | SNP | G | G | A |
| NCKAP1 | SNP | A | A | T |
| NIPAL4 | SNP | A | A | G |
| NRXN2 | SNP | G | G | C |
| OBSCN | SNP | G | G | A |
| OR4C6 | SNP | C | C | A |
| OR4C6 | SNP | A | A | G |
| OR4N5 | SNP | C | C | T |
| OR52N2 | SNP | C | C | G |
| P4HA3 | SNP | A | A | G |
| PCSK2 | SNP | C | C | T |
| PDCD11 | SNP | A | A | G |
| PDE6C | SNP | A | A | G |
| PDE6H | SNP | G | G | A |
| PNOC | SNP | A | A | T |
| POU5F1 | SNP | C | C | T |
| PRKAA2 | SNP | C | C | T |
| RBL2 | SNP | C | C | T |
| RPS7 | SNP | C | C | T |
| SH3TC2 | SNP | C | C | T |
| SLCO3A1 | SNP | G | G | A |
| SNAP25 | SNP | A | A | C |
| SPATA17 | SNP | T | T | A |
| SYNPO2 | SNP | C | C | A |
| SYT3 | SNP | G | G | A |
| TMEM222 | SNP | T | T | C |
| TRIP10 | SNP | C | C | T |
| VPS13B | SNP | A | A | G |
| VWDE | SNP | T | T | A |
| WDFY3 | SNP | T | T | A |
| ZCCHC10 | SNP | G | G | T |
| loss_11q23.1 | CNV | None | None | None |
| SF3B1 | SNP | T | T | C |
| gain_MCL1 | CNV | NA | NA | NA |
| CYB5RL | DEL | CTCTTGGTAA | CTCTTGGTAA | — |
| gain_8q | CNV | None | None | None |
| homdel_STK11 | CNV | NA | NA | NA |
| TP53 | DEL | TGTGGAATCAA | TGTGGAATCAA | — |
| | | CCCACAGCTGC | CCCACAGCTGC | |
| | | ACAGGGCAGGT | ACAGGGCAGGT | |
| | | CTTGGC | CTTGGC | |
| homdel_APC | CNV | NA | NA | NA |
| gain_IGF1R | CNV | NA | NA | NA |
| loss_14q32.33 | CNV | None | None | None |
| loss_8p | CNV | None | None | None |
| homdel_14q32.33 | CNV | NA | NA | NA |
| gain_11q22.2 | CNV | NA | NA | NA |
| homdel_RB1 | CNV | NA | NA | NA |
| loss_19q | CNV | NA | NA | NA |
| loss_19p | CNV | NA | NA | NA |
| loss_6p | CNV | NA | NA | NA |
| loss_3p26.3 | CNV | NA | NA | NA |
| loss_17p | CNV | None | None | None |
| homdel_15q15.1 | CNV | None | None | None |
| EIF4A1 | DEL | GA | GA | — |
| Unknown | SNP | G | G | A |
| AC015849.16 | SNP | C | C | T |
| AC015849.16 | SNP | A | A | G |
| AHNAK | SNP | C | C | A |
| ANPEP | SNP | A | A | T |
| ATP4A | SNP | G | G | A |
| B3GALT2 | SNP | G | G | T |
| BHLHE40 | SNP | C | C | T |
| BUB1 | SNP | C | C | A |
| C17orf70 | SNP | G | G | A |
| C1orf101 | SNP | T | T | C |
| C1orf141 | SNP | C | C | A |
| C8orf37 | SNP | C | C | T |
| CEP152 | SNP | T | T | A |
| CHD2 | SNP | G | G | T |
| CHRNB2 | SNP | G | G | A |
| COL12A1 | SNP | T | T | C |
| CPZ | SNP | G | G | A |
| CRIM1 | SNP | T | T | C |
| CRIP2 | SNP | G | G | A |
| DKK3 | SNP | C | C | A |
| DNAH8 | SNP | C | C | T |
| DOCK1 | SNP | G | G | A |
| FAR2 | SNP | C | C | A |
| FLNB | SNP | G | G | T |
| FTMT | SNP | C | C | A |
| GAL3ST2 | SNP | A | A | G |
| GIT1 | SNP | A | A | T |
| GUCA2A | SNP | C | C | T |
| IGHG1 | SNP | G | G | C |
| IGHJ6 | SNP | C | C | T |
| IGHJ6 | SNP | C | C | G |
| IGHM | SNP | C | C | T |
| IGHV2-26 | SNP | G | G | A |
| IGHV3-23 | SNP | G | G | A |
| IGHV3-23 | SNP | A | A | G |
| IGHV3-23 | SNP | C | C | T |
| IGLV3-21 | SNP | A | A | G |
| IGLV3-21 | SNP | C | C | A |
| IGLV3-21 | SNP | C | C | T |
| IGLV4-3 | SNP | G | G | A |
| IGLV4-3 | SNP | G | G | C |
| IGLV4-3 | SNP | C | C | T |
| IGLV4-3 | SNP | C | C | G |
| IGLV4-3 | SNP | G | G | T |
| LAMB4 | SNP | G | G | A |
| LPPR4 | SNP | C | C | T |
| LRP1 | SNP | C | C | T |
| MEFV | SNP | G | G | A |
| MLH3 | SNP | G | G | A |
| MMP16 | SNP | C | C | A |
| MYH7 | SNP | C | C | T |
| NAALADL2 | SNP | G | G | A |
| NLRC5 | SNP | G | G | A |
| NPAT | SNP | A | A | G |
| NRXN3 | SNP | T | T | A |
| NUAK1 | SNP | A | A | T |
| OR4K14 | SNP | C | C | A |
| PAQR4 | SNP | C | C | T |
| PCLO | SNP | C | C | A |
| PLA2G2E | SNP | G | G | T |
| PLCB1 | SNP | G | G | T |
| PPA1 | SNP | C | C | T |
| RAPGEF2 | SNP | C | C | A |
| RCC2 | SNP | G | G | T |
| REEP6 | SNP | G | G | T |
| RGMA | SNP | G | G | A |
| RING1 | SNP | G | G | A |
| RNF213 | SNP | G | G | A |
| SALL1 | SNP | G | G | A |
| SCNN1A | SNP | G | G | T |
| SLC35E2B | SNP | C | C | T |
| SNAP91 | SNP | C | C | T |
| SNAP91 | SNP | A | A | G |
| SNCAIP | SNP | G | G | T |
| SPDYE4 | SNP | G | G | A |
| SYMPK | SNP | C | C | T |
| TENM3 | SNP | G | G | A |
| TIMD4 | SNP | G | G | A |
| TMEM178A | SNP | C | C | T |
| TMEM216 | SNP | T | T | A |
| TMEM242 | SNP | G | G | A |
| TMPRSS9 | SNP | C | C | T |
| TRIM27 | SNP | T | T | C |
| TRIML2 | SNP | G | G | A |
| UGT3A1 | SNP | C | C | T |
| WDR46 | SNP | G | G | C |
| ZFP36L2 | SNP | G | G | A |
| ZNF488 | SNP | A | A | T |
| SF3B1 | SNP | T | T | c |
| SF3B1 | SNP | T | T | G |
| SF3B1 | SNP | C | C | A |
| TP53 | SNP | C | C | G |
| gain_8q24.3 | CNV | NA | NA | NA |
| ADNP2 | DEL | AG | AG | — |
| loss_9p21.2 | CNV | NA | NA | NA |
| loss_9p21.3 | CNV | NA | NA | NA |
| SEC16A | DEL | CGAGAAGG | CGAGAAGG | — |
| homdel_14q32.33 | CNV | None | None | None |
| loss_18p | CNV | NA | NA | NA |
| gain_MYC | CNV | NA | NA | NA |
| loss_9q34.3 | CNV | NA | NA | NA |
| gain_1q23.3 | CNV | NA | NA | NA |
| gain_22q11.21 | CNV | NA | NA | NA |
| loss_5p | CNV | NA | NA | NA |
| loss_14q | CNV | NA | NA | NA |
| loss_5q | CNV | NA | NA | NA |
| C5orf45 | DEL | GGAATGAGATT | GGAATGAGATT | — |
| | | CTATGGACTTG | CTATGGACTTG | |
| | | GA | GA | |
| DNAH6 | DEL | TACAC | TACAC | — |
| loss_8p11.21 | CNV | NA | NA | NA |
| gain_4q | CNV | NA | NA | NA |
| gain_4p | CNV | NA | NA | NA |
| loss_18q23 | CNV | NA | NA | NA |
| loss_9p24.1 | CNV | NA | NA | NA |
| ZFC3H1 | DEL | AAGACAGGGAA | AAGACAGGGAA | — |
| | | GCATAGAAGT | GCATAGAAGT | |
| loss_PARK2 | CNV | NA | NA | NA |
| gain_MCL1 | CNV | NA | NA | NA |
| LIFR | DEL | A | A | — |
| loss_4q | CNV | NA | NA | NA |
| gain_8q | CNV | None | None | None |
| gain_8p | CNV | NA | NA | NA |
| loss_4p | CNV | NA | NA | NA |
| gain_10q | CNV | NA | NA | NA |
| gain_18p | CNV | NA | NA | NA |
| PDE6A | DEL | CCAGAATTCA | CCAGAATTCA | — |
| C10orf137 | DEL | C | C | — |
| gain_8q23.3 | CNV | NA | NA | NA |
| loss_RB1 | CNV | NA | NA | NA |
| loss_10q | CNV | NA | NA | NA |
| gain_8q22.2 | CNV | NA | NA | NA |
| loss_8p | CNV | NA | NA | NA |
| RAB4B-EGLN2 | DEL | AGAG | AGAG | — |
| gain_13q | CNV | NA | NA | NA |
| CHD2 | DEL | G | G | — |
| DDX11 | DEL | GTCCCTG | GTCCCTG | — |
| gain_7q36.3 | CNV | NA | NA | NA |
| MYB | DEL | GTGAC | GTGAC | — |
| homdel_RB1 | CNV | None | None | None |
| gain_6q | CNV | NA | NA | NA |
| SRSF12 | DEL | AATACATATAT | AATACATATAT | |
| | | GTATAAGCACG | GTATAAGCACG | |
| | | CATA | CATA | |
| gain_7q | CNV | NA | NA | NA |
| gain_7p | CNV | NA | NA | NA |
| loss_6p | CNV | NA | NA | NA |
| loss_17p13.3 | CNV | None | None | None |
| loss_17p | CNV | NA | NA | NA |
| loss_10p15.3 | CNV | NA | NA | NA |
| gain_6p | CNV | NA | NA | NA |
| gain_5q | CNV | NA | NA | NA |
| loss_21q | CNV | NA | NA | NA |
| MTR | DEL | TAAAA | TAAAA | — |
| ANGPTL6 | SNP | C | C | T |
| BCAR1 | SNP | G | G | A |
| BHLHA15 | SNP | G | G | A |
| BID | SNP | C | C | T |
| C10orf11 | SNP | C | C | A |
| C7 | SNP | G | G | A |
| CEND1 | SNP | G | G | T |
| CXCR2 | SNP | C | C | T |
| DEFB114 | SNP | C | C | T |
| DNAH5 | SNP | C | C | T |
| DNHD1 | SNP | G | G | A |
| EDNRA | SNP | C | C | T |
| EXOC6B | SNP | A | A | T |
| GRM5 | SNP | T | T | C |
| HADH | SNP | T | T | C |
| IRF8 | SNP | A | A | T |
| KIF20B | SNP | A | A | G |
| LPHN3 | SNP | G | G | A |
| MPP5 | SNP | C | C | T |
| MT1A | SNP | G | G | C |
| MTIF3 | SNP | C | C | T |
| NAV2 | SNP | C | C | A |
| NCKAP5 | SNP | G | G | A |
| NDRG1 | SNP | C | C | T |
| PDZRN4 | SNP | G | G | A |
| RBM38 | SNP | A | A | T |
| RD3 | SNP | G | G | A |
| RIOK1 | SNP | A | A | G |
| ROBO4 | SNP | G | G | A |
| SAMHD1 | SNP | C | C | T |
| SAMHD1 | SNP | C | C | T |
| SEMA4G | SNP | G | G | A |
| SEMA6D | SNP | A | A | G |
| SLC25A10 | SNP | G | G | A |
| SPATS2 | SNP | C | C | T |
| TENM2 | SNP | G | G | A |
| TMEM176B | SNP | T | T | C |
| TMPRSS15 | SNP | A | A | G |
| TNIP3 | SNP | G | G | A |
| TRAF5 | SNP | C | C | G |
| TRBC2 | SNP | C | C | T |
| ZNF16 | SNP | G | G | T |
| ZNF292 | SNP | C | C | A |
| ZNF296 | SNP | G | G | A |
| ZNF629 | SNP | G | G | A |
| FAT1 | SNP | C | C | T |
| KEL | SNP | C | C | T |
| NRAS | SNP | T | T | C |
| loss_14q32.33 | CNV | None | None | None |
| SAMHD1 | DEL | TAGAAGG | TAGAAGG | — |
| IRF8 | DEL | TCATTTT | TCATTTT | — |
| homdel_APC | CNV | None | None | None |
| homdel_14q32.33 | CNV | NA | NA | NA |
| HAL | DEL | T | T | — |
| NOTCH1 | DEL | AG | AG | — |
| XPO4 | DEL | CTGAAAT | CTGAAAT | — |
| SAMHD1 | INS | — | — | A |
| RAI1 | INS | — | — | G |
| ABCA13 | SNP | T | T | C |
| ANO5 | SNP | T | T | A |
| ASXL3 | SNP | T | T | C |
| ATP12A | SNP | G | G | A |
| BRSK2 | SNP | G | G | C |
| C17orf62 | SNP | T | T | C |
| CDK5R1 | SNP | C | C | T |
| COL12A1 | SNP | C | C | T |
| COX15 | SNP | G | G | A |
| CTD-3064M3.7 | SNP | G | G | A |
| DROSHA | SNP | C | C | T |
| DSEL | SNP | G | G | T |
| FAM120A | SNP | G | G | C |
| FSIP2 | SNP | T | T | A |
| GALNT5 | SNP | G | G | C |
| GPT | SNP | G | G | A |
| GRIK3 | SNP | G | G | A |
| IRAK1BP1 | SNP | A | A | T |
| MIR518C | SNP | C | C | T |
| MYH9 | SNP | C | C | T |
| NXF1 | SNP | T | T | A |
| OR5AR1 | SNP | A | A | G |
| OR5J2 | SNP | A | A | T |
| PDIA3 | SNP | A | A | G |
| PGLYRP3 | SNP | G | G | A |
| RCC1 | SNP | C | C | T |
| RP11-343C2.12 | SNP | A | A | G |
| RPL22L1 | SNP | A | A | G |
| SLC12A5 | SNP | C | C | T |
| SNHG14 | SNP | G | G | A |
| TENM3 | SNP | A | A | T |
| TOM1L2 | SNP | G | G | A |
| TP53 | SNP | C | C | A |
| TP53 | SNP | T | T | A |
| TRIM37 | SNP | G | G | T |
| TRIP4 | SNP | G | G | T |
| TUBGCP2 | SNP | T | T | C |
| ZNF12 | SNP | G | G | A |
| ZNF254 | SNP | C | C | G |
| ZNF560 | SNP | G | G | A |
| loss_14q32.33 | CNV | None | None | None |
| PTPN11 | SNP | A | A | T |
| SF3B1 | SNP | C | C | T |
| SF3B1 | SNP | T | T | G |
| TP53 | SNP | G | G | A |
| TP53 | SNP | T | T | A |
| TP53 | SNP | T | T | C |
| homdel_RB1 | CNV | NA | NA | NA |
| TC2N | INS | — | — | AA |
| gain_22q11.21 | CNV | NA | NA | NA |
| loss_11p15.5 | CNV | None | None | None |
| SYNE1 | INS | — | — | C |
| homdel_APC | CNV | NA | NA | NA |
| BAZ2A | DEL | AG | AG | — |

While discrete gene mutations of the target of therapeutic inhibitors have been long-identified as a common mechanism of resistance to targeted therapies²⁸⁻³⁰, no recurrent mutations of BCL-2 family members have been yet reported in limited series thus far²⁷, nor within Applicants' small patient cohort. Rather, Applicants' studies implicate major clonal shifts, alterations in the expression profiles of cells, and more complex genomic changes in venetoclax resistance. On the one hand, Applicants' aggregate results underscore that alterations leading to overexpression of MCL-1, and therefore the ability to sequester BIM and therefore to compensate BCL-2 inhibition, are the most consistent adaptive mechanism of resistance for this drug.^{15,31,32} On the other hand, Applicants' genome-wide screens together with functional characterization of a resistant cell line have implicated deregulated metabolism via altered expression of components of the AMPK signaling pathway in this process. In support of the notion of a dual impact of venetoclax on the mitochondria, Applicants notably identified a recurrence of amp(1q), encompassing both MCL1 and the AMPK pathway component PRKAB2 in both representative cell lines and in patient samples. Applicants' work thus characterizes venetoclax resistance as involving both reprogramming of the outer membrane biology of the mitochondria, leading to expression changes in BCL-2 family members, as well as increased OXPHOS activity which is resident in the organelle's inner membrane (FIG. 6e).

Applicants' findings thus demonstrate that venetoclax imposes a broader scope of ‘mitochondrial stress’ than previously suggested. Through this lens, Applicants' study casts new light on the mechanisms underlying diverse metabolic strategies previously evaluated to overcome resistance to BCL-2 inhibition, which have included hypoxia 33, caloric restriction 4 or the use of the kinase inhibitors (including of AMPK), such as sunitinib^{35,36}. First, Applicants find that at least in part, the regulation of such optimized metabolism depends on repression of the ID family of lymphoid transcriptional regulators. These results mesh well with recent work demonstrating that lymphoid transcription factors function as metabolic gatekeepers by limiting the amount of cellular ATP to levels that are insufficient for malignant transformation.²⁰ Hence, although Applicants' studies focused on the impact of ID3, a broader involvement of altered lymphoid differentiation state on venetoclax resistance is anticipated since repression of additional lymphoid transcription factors (IKZF5, EP300) were identified in Applicants' knockout screen. Second, Applicants' work adds to the growing evidence of crosstalk between the BCL-2 family and the mETC. Early studies demonstrated that even prior to downstream activation of caspases, the apoptosis program results in a defect in the mETC³⁷. Maintenance of mETC integrity was recently shown to allow cells to proliferate after apoptosis induction.³⁸. Conversely, the BCL-2 family proteins can participate in non-apoptotic functions such as regulation of mitochondrial morphology, autophagy or metabolism.³⁹⁻⁴²

These new insights have the potential to provide new therapeutic opportunities. Applicants point out that mutations affecting these factors have been detected across B-cell malignancies, and thus Applicants expect them to confer venetoclax resistance. Applicants' findings may therefore guide the further development of BCL-2 inhibitors in diseases lacking these mutations. Applicants' data support the idea that targeting MCL-1 with emerging inhibitors would be a rationale and effective way to counteract venetoclax resistance. Given the association with higher OXPHOS status with resistance, this adaptive activity can be exploited to overcome venetoclax resistance.

Methods are provided below.

Human samples. Genomic DNA was isolated (DNAeasy Blood and Tissue Kit, Qiagen) from specimens collected from CLL patients enrolled on clinical trials of venetoclax treatment (NCT01328626, NCT02141282), approved by and conducted in accordance with the principles of the Declaration of Helsinki and with the approval of the Institutional Review Boards (IRB) of the University of Texas/MD Anderson Cancer Center (MDACC; Patients 1, 3, 4) or of Dana-Farber Cancer Institute (DFCI; Patient 2, 5, 6). Blood and/or tissue tumor samples were collected at baseline, before initiation of venetoclax therapy, and at relapse or progression on venetoclax.

Cells lines. OCI-Ly1 cells (DSMZ, Braunschweig Germany) were cultured in Iscove's Modified Dulbecco's Media (Gibco) supplemented with 10% fetal bovine serum (FBS) and 1% penicillin/streptomycin/glutamine. An OCI-Ly1 cell line resistant to venetoclax was generated over 10 weeks by exposing the cells to increasing doses of venetoclax starting at 10 nM, and then doubling this dose when the cells were able to grow at a rate equivalent to the parental lines until the cells were able to tolerate 1 μM of venetoclax.

Drugs. Venetoclax (ABT-199; Selleck Chemicals), dorsomorphin (Sigma), oligomycin A (Sigma), antimycin A (Sigma), and the MCL-1 inhibitor S63845 (Chemietek) were used for drug treatment experiments. All drugs were resuspended in DMSO (Sigma).

Cell viability assay. The Cell Titer-Glo Luminescent Cell Viability Assay (Promega) was used to determine the relative number of viable cells after drug treatment. 0.2×10⁶ cells/mL were seeded in a 24 well-plate and treated with drugs for 24 or 48 hours. The viability assay was conducted using the manufacturer's protocol after treatment. Values were normalized to DMSO-treated cells. Plates were read on a SpectraMax M3 reader (Molecular Devices).

Western blotting. Total protein from cells was isolated using RIPA Buffer (20 mM Tris-HCl pH 7.5, 150 mM NaCl, 1 mM Na₂EDTA, 1 mM EGTA, 1% NP-40, 1% sodium deoxycholate, 2.5 mM sodium pyrophosphate, 1 mM 8-glycerophosphate, 1 mM Na₃VO₄, 1 ug/mL leupeptin), supplemented with protease inhibitors (Thermo Fisher Scientific) and phosphatase inhibitors (Thermo Fisher Scientific). Protein concentration was determined with the BCA Protein Assay (Pierce). Protein samples (25 μg) were separated on either 4-12% Bis-Tris gels (proteins <250 kDa) or Tris-acetate gels (proteins >250 kDa). Protein was transferred to a nitrocellulose or PVDF membrane (Life Technologies) using the iBlot2 system (Life Technologies). Membranes were incubated overnight with primary antibodies recognizing BCL-2 (1:1000; Abcam), MCL-1 (1:200; Santa Cruz), BCL-XL (1:100; Santa Cruz), BIM (1:1000; Cell Signaling Technology), BAK (1:1000; Cell Signaling Technology), BAX (1:1000; Cell Signaling Technology), Pegasus (1:1000; Santa Cruz), OTUD5 (1:1000; Cell Signaling Technology), NOXA (1:100; Santa Cruz), ID3 (1:1000; Cell Signaling Technology), ID2 (1:1000; Cell Signaling Technology), p300 (1:1000; Santa Cruz), UBR5 (1:1000; Cell Signaling Technology), IκBα (1:1000; Cell Signaling Technology), NF-1 (1:200; Santa Cruz), AMPKα (1:1000; Cell Signaling Technology), PKA beta (1:500, Abcam) and GAPDH (1:1000; Cell Signaling Technology). After incubation in the appropriate secondary antibodies (anti-rabbit IgG, 1:5000, HRP-linked, Millipore; anti-mouse IgG, 1:1000, HRP-linked, GE Healthcare UK), protein was detected using chemiluminescence (Western Lightning Plus-ECL, Perkin Elmer).

Genome-scale screens: Conduct of the genome-wide CRISPR-screen. The strategy used was similar in approach as previously reported 4. 300×10⁶ Cas9-OCI-Ly1 cells were suspended in media supplemented with 8 μg/mL polybrene and seeded into 9 12-well plates (1 mL per well). Titration of the dose of puromycin and of polybrene on OCI-Ly1 cells was undertaken to achieve 100% and minimal death of non-infected cells, respectively. The BRUNELLO sgRNA viral library in lentiGuide-puro (Genetic Perturbation Platform, Broad Institute) was added to each well (200 μL/mL), titrated to achieve an infection rate of 30% without excessive cell death and to minimize multiplicity of infection. The plates were spun at 2000 rpm for 2 h at 37° C. and incubated at 37° C. for 24 h. Polybrene was diluted by adding 2 mL of standard media to each well. Puromycin selection (1 μg/mL) was initiated 48 hours post-transduction and sustained for 5 days. Two days after puromycin selection, transduced OCI-Ly1 cells were treated with venetoclax (100 nM—a dose identified to be growth suppressive at day 14) or DMSO as control for 14 days in T225 flasks. Cells were counted and re-split every three days, maintaining a concentration of 200,000 cells/mL Approximately 40 million cells were frozen before and after venetoclax or DMSO selection for sequencing. This experiment was performed in duplicates.

Genomic DNA (gDNA) was isolated (Maxiprep kits, Qiagen), and PCR and barcoded sgRNA or ORF-sequencing were performed, as previously described.⁸ Samples were sequenced on a HiSeq2000 (Illumina). For analysis, the read counts were normalized to reads per million and then log 2 transformed. The log 2 fold-change of each sgRNAs was determined relative to the initial time point for each. Significance of the sgRNAs' enrichment was assessed using the STARS software (v1.3, Broad Institute).

Conduct of the genome-wide ORF-screen. The approach used was based on previous reports^{44,45}. Optimal transduction conditions were determined in order to achieve 30-50% infection efficiency, corresponding to a multiplicity of infection (MOI) of ˜0.5-1. Briefly, 1.5×10⁶ of OCI-Ly1 cells were spun-infected in 6 wells of a 12-well plates with different virus volumes. The day after, OCI-Ly1 cells were seeded in 2 wells of a 6-well plate, each with complete medium, one supplemented with 1 μg/mL of puromycin. Cells were counted 4 days post-selection to determine the infection efficiency, comparing survival with and without puromycin selection. Volumes of virus that yielded ˜30-50% infection efficiency were used for screening.

Applicants used an ORFeome barcoded library that contains 17,255 barcoded ORFs overexpressing 12,952 unique genes (Broad Institute). Large-scale infections were performed in 12-well format as the viral titration described above using the optimized volume of virus, and pooled 24 hours post-centrifugation. Infections were performed with an adequate number of cells to achieve a representation of at least 1000 cells per ORF following puromycin selection (˜2×10⁷ surviving cells containing 17,255 ORFs). ˜24 hours after infection, all wells within a replicate were pooled and were split into T225 flasks. 48 hours after infection, cells were selected with puromycin for 72 hours to remove uninfected cells. Seventy-two hours after selection, OCI-Ly1 cells were treated with either DMSO or 100 nM venetoclax and passaged in fresh media containing either DMSO or drug every 3-4 days. Cells were harvested 10 days after initiation of treatment. Isolation of genomic DNA, sequencing and analyses were performed as for the CRISPR-Cas9 screen. The log 2 fold-change of each ORF was determined relative to the initial time point for each. This experiment was performed in duplicates.

Generation of CRISPR-Cas9 engineered isogenic cell lines and overexpression cell lines was as described below.

Cloning of sgRNA vectors. Two of 4 sgRNAs per target were selected from the BRUNELLO genome-scale library (based on highest levels of representation from the genome-wide screen) and related DNA oligonucleotides were synthesized (Gene Link; Table 1), along with oligonucleotides corresponding to 2 control non-targeting sgRNAs per gene. Oligonucleotides were phosphorylated and annealed using T4 PNK (New England Biolabs). The backbone vector (pLKO5.sgRNA.EFS.GFP, Addgene #57822) was digested with FastDigest BsmBI (Thermo Scientific), and the vector and oligonucleotides were ligated with T7 DNA ligase (New England Biolabs). The ligation reaction was treated with Plasmid-Safe exonuclease (Epicentre) to prevent unwanted recombination products. The final product (1 μL) was transformed into 25 μL of DH5a competent cells (New England Biolabs). Colonies were selected and sequenced before undergoing plasmid DNA extraction (Endotoxin-Free Plasmid Maxiprep, Qiagen).

Cloning of ORFs vectors. The ORFs for PRKAR2B and PRKAA2 (clone ID: TRCN0000480583 and TRCN0000492160, respectively; Broad Institute Genetic Perturbation Platform ORFeome library (https://portals.broadinstitute.org/gpp/public/)) were cloned into the pLX_TRC317. This is a lentiviral expression vector that encodes a puromycin resistance cassette and an ORF expression cassette under control of the EF1-alpha promoter.

Generation of a Cas9 overexpressing cell line. Stable Cas9-expressing OCI-Ly1 cells were generated by transducing parental cells with lentivirus prepared with lentiCas9-Blast pXR101 (Addgene plasmid #52962)⁴⁶ encoding Cas9 and blasticidin resistance. Selection with blasticidin (10 μg/mL) was initiated 48 h after transduction and sustained throughout culture of Cas9-expressing cell lines. Cas9 activity was checked as previously reported by using the pXPR-011 vector (Addgene plasmid #52702).47

Lentivirus production and purification. To produce lentivirus, ˜800,000 HEK293T cells were seeded per well in a 6-well plate in 2.7 mL of antibiotic-free DMEM supplemented with 10% FBS. For each well, 150 μL of OptiMEM (Life Technologies) was mixed with 5 μg of pLKO5_sgRNA plasmid, 0.4 μg of pVSV.G, and 1.5 μg of psPAX2 (Addgene #12260). Separately, 9 μl of Lipofectamine 2000 (Life Technologies) was diluted in 150 μl OptiMEM. After a 15 min incubation at room temperature, the DNA and Lipofectamine mixes were combined and incubated together at room temperature for 30 min before being added to the cells. After 12 h, the media was changed to DMEM supplemented with 20% FBS. 48 h post-transfection, 3 mL of media was removed and filtered through a 0.45 μm low protein binding membrane (Millipore Steriflip HV/PVDF) and added to 1 mL of LentiX Concentrator (Clontech). This mixture was then incubated at 4° C. for 2 h, and centrifuged at 1500×g for 45 min at 4° C. The pellet was resuspended in 100 μL of PBS and stored in aliquots at −80° C.

Generation of engineered cell lines. 0.5×10⁶ target OCI-Ly1 cells were suspended in media supplemented with 8 μg/mL polybrene and seeded into 6-well plates (1 mL per well), to which lentivirus was added (50 μL/mL to each well). The plates were spun at 2000 rpm for 2 h at 37° C. and incubated at 37° C. for 24 h. The polybrene-containing media was then replaced by 2 mL of fresh media per well. After 3 days, transduced cells were selected (i.e. by puromycin (1 μg/mL) for 1 week for the ORF-overexpressing cells) or sorted (i.e. based GFP or mCherry expression for the CRISPR gene-edited lines) and cryopreserved for further experiments. As confirmation that the engineered cell lines expressed the expected alterations, Applicants: (i) evaluated the expression of the targeted protein by western-blotting (FIG. 6d), and (ii) for the CRISPR-Cas9 engineered cell lines, performed targeted DNA sequencing for the CRISPR target sites before and after 2 weeks of exposure to venetoclax (100 nM), and assessed the proportion of frameshift indels (FIG. 6g). In brief, Applicants used a two-step touchdown PCR protocol. Genomic DNA from the pre- and post-treated samples was PCR-amplified using KAPA HiFi DNA polymerase and primers specific for the target sequence of the gRNAs. Products from the first reaction were barcoded with Illumina sequencing adaptor sequences and indexes during a second round of PCR. Following PCR, samples were purified with Agencourt AMPure XP beads (Beckman Coulter) and quantified on a Bioanalyzer (Agilent) with High Sensitivity DNA chips. Sample libraries were diluted to 4 nM, pooled, and ran on the Illumina MiSeq platform using single-end sequencing with the following parameters: read 1: 296nt, index 1: 6nt.

Single-cell cloning of the ID3 knockout. OCI-Ly1 cells was performed using dose-limiting dilution strategy. Cells from the bulk ID3 OCI-Ly1 were seeded at a concentration of 0.5 cells/well in a 96-well plate (5 plates per cell line). To investigate the presence of mutations, 6 clones per sgRNA were analyzed by PCR and Sanger sequencing using primers flanking the target sites for the sgRNAs (Forward primer: 5′-TGACAAGTTCCGGAGTGAGC-3′ (SEQ ID NO:1); Reverse:5′-CGGTATCAGCGCTTCCTCAT-3′ (SEQ ID NO:2)). The absence of ID3 protein were confirmed in clones harboring loss-of-function mutations by western blot (FIG. 8d). Three different knockout single-cell clones for each sgRNA were used for further functional studies.

Whole-exome sequencing and data analyses. Library construction from CLL and matched germline DNA of Patients 1-6 was performed as previously described 48, with the following modifications: (i) initial genomic DNA input into shearing was reduced from 3 μg to 10-100 ng in 50 μl of solution; (ii) for adapter ligation, Illumina paired-end adapters were replaced with palindromic forked adapters (from Integrated DNA Technologies), with unique dual-indexed molecular barcode sequences included in the adapter sequence to facilitate downstream pooling. With the exception of the palindromic forked adapters, the reagents used for end repair, A-base addition, adapter ligation and library enrichment PCR were purchased from KAPA Biosciences in 96-reaction kits, (iii) during the post-enrichment solid-phase reversible immobilization (SPRI) cleanup, elution volumes were reduced to 30 μL to maximize library concentration, and a vortexing step was added to maximize the amount of template eluted. Any libraries with concentrations below 40 ng/ml (per PicoGreen assay, automated on an Agilent Bravo) were considered failures and reworked from the start of the protocol. Following library construction, hybridization and capture were performed using the relevant components of Illumina's Nextera Rapid Capture Exome Kit and following the manufacturer's suggested protocol. with the following exceptions: first, all libraries within a library construction plate were pooled prior to hybridization. Second, the Midi plate from Illumina's Nextera Rapid Capture Exome Kit was replaced with a skirted PCR plate to facilitate automation. All hybridization and capture steps were automated on the Agilent Bravo liquid handling system. After post-capture enrichment, library pools were quantified using qPCR (automated assay on the Agilent Bravo), using a kit purchased from KAPA Biosystems with probes specific to the ends of the adapters. On the basis of qPCR quantification, libraries were normalized to 2 nM, and then denatured using 0.1N NaOH on the Hamilton Starlet. After denaturation, libraries were diluted to 20 pM using hybridization buffer purchased from Illumina.

Cluster amplification of denatured templates was performed according to the manufacturer's protocol (Illumina) using HiSeq 4000 cluster chemistry and HiSeq 4000 flowcells. The flowcells are then analyzed using RTA v.1.18.64 or later. Each pool of whole exome libraries was run on paired 76 bp runs, reading the dual-indexed sequences to identify molecular indices and sequenced across the number of lanes needed to meet coverage for all libraries in the pool.

Alignments to hg19 using bwa version 0.5.9-r16⁴⁹ and quality control were performed using the Picard (http://picard.sourceforge.net/) and Firehose (pipelines at the Broad Institute). Firehose is a framework combining workflows for the analysis of cancer-sequencing data. The workflows perform quality control, local re-alignment, mutation calling, small insertion and deletion identification, rearrangement detection and coverage calculations, among other analyses. A dbGaP accession number for the depositing of WES data for this study is pending.

Identification of somatic mutations. Sequencing output was processed with the Picard and GATK toolkits developed at the Broad Institute, a process that involves marking duplicate reads, recalibrating base qualities and realigning around somatic small insertions and deletions (sINDELs). All BAM files were generated by aligning with bwa version 0.5.9 to the NCBI Human Reference Genome Build hg19. Prior to variant calling, the impact of oxidative damage (oxoG) and FFPE damage to DNA during sequencing was quantified according to Costello et al.⁵⁰ The cross-sample contamination was measured with ContEst based on the allele fraction of homozygous SNPs⁵¹, and this measurement was used in MuTect. From the aligned BAM files, somatic alterations were identified using a set of tools developed at the Broad Institute (www.broadinstitute.org/cancer/cga). The details of Applicants' sequencing data processing have been described elsewhere^{52,53}.

Following Applicants' standard procedure, sSNVs were detected using MuTect⁹ (version 1.1.6); sINDELs were detected using Strelka⁵⁴. Applicants then applied a stringent set of filters to improve the specificity of Applicants' sSNV and sINDEL calls and remove likely FFPE artifacts. Applicants applied an allele fraction specific panel-of-normals filter, which compares the detected variants to a large panel of normal exomes and removes variants that were observed in the panel-of-normals. Applicants then applied a realignment based filter, which removes variants that can be attributed entirely to ambiguously mapped reads. All filtered events in candidate CLL genes were also manually reviewed using the Integrated Genomics Viewer (IGV)⁵⁵. In the matched sample sets from 6 individuals, Applicants utilized “forced calling” to quantify the number of reads supporting the alternate and reference alleles at sites which were detected in any sample from that individual. Estimation of and correction for tumour contamination in normal was performed using the deTiN algorithm⁵⁶ to recover somatic mutations that would have otherwise been filtered out due to evidence of the mutation in the normal. To address the lack of a matched normal sample (in Patient 3) Applicants used a stringent panel-of-normals and population allele frequency criteria, and excluded non-coding variants from analysis. To address the lack of a matched normal sample (in Patient 6), Applicants used a stringent panel-of-normals and population allele frequency criteria, and excluded non-coding variants from analysis. Furthermore, parental OCI-Ly1-S cells were used as a source control DNA in order to highlight sSNVs that were acquired in the resistant OCI-Ly1-R cells. Reference lists for sSNVs and sINDELs in known putative CLL driver genes as well as for recurrent CNAs were concatenated based on previous sequencing studies of large CLL cohorts⁵⁷⁻⁶¹. Total copy number was measured using ReCapSeg (www.broadinstitute.org/cancer/cga), then segmented into allelic copy number with AllelicCapSeg⁶² based on heterozygous germline sites detected with HaplotypeCaller according to the protocol described previously (http://archive.broadinstitute.org/cancer/cga/acsbeta).

Estimation of mutation cancer cell fraction using ABSOLUTE and clonal evolution mapping. The cancer cell fraction (represented as a probability density distribution∈[0, 1]) of individual somatic alterations were estimated using the ABSOLUTE algorithm (v1.5) which calculates the sample purity, ploidy, and local absolute DNA copy-number of each mutation, as previously described^{59,63}. CCFs were clustered as previously described⁵⁹ to delineate distinct subclonal populations. Phylogenetic relationships between these populations were inferred using patterns of shared mutations and CCF using the PhylogicNDT analysis.

RNA sequencing and cDNA Library Construction. Total RNA was quantified using the Quant-iT™ RiboGreen® RNA Assay Kit and normalized to 5 ng/μl. Following plating, 2 μL of ERCC controls (using a 1:1000 dilution) were spiked into each sample. An aliquot of 200 ng for each sample was transferred into library preparation which uses an automated variant of the Illumina TruSeq™ Stranded mRNA Sample Preparation Kit. This method preserves strand orientation of the RNA transcript. It uses oligo dT beads to select mRNA from the total RNA sample, followed by heat fragmentation and cDNA synthesis from the RNA template. The resultant 400 bp cDNA then goes through dual-indexed library preparation: ‘A’ base addition, adapter ligation using P7 adapters, and PCR enrichment using P5 adapters. After enrichment, the libraries were quantified using Quant-iT PicoGreen (1:200 dilution). After normalizing samples to 5 ng/μL, the set was pooled and quantified using the KAPA Library Quantification Kit for Illumina Sequencing Platforms. The entire process was in a 96-well format and all pipetting is done by either Agilent Bravo or Hamilton Starlet.

Illumina Sequencing. Pooled libraries were normalized to 2 nM and denatured using 0.1 N NaOH prior to sequencing. Flowcell cluster amplification and sequencing were performed according to the manufacturer's protocols using either the HiSeq 2000 or HiSeq 2500 instrument. Each run generated a 101 bp paired-end with an eight-base index barcode read. Data was analyzed using the Broad Picard Pipeline, which includes de-multiplexing and data aggregation.

Data analyses. RNA-seq data were aligned to GRCh38.p5 with STAR-2.5.1b.⁶⁴ Gene expression was quantified with RSEM-1.2.31.⁶⁵ DESeq2⁶⁶ was applied to call differentially expressed genes between each cell line and control group. Pathway enrichment analysis was performed with GSEA⁶⁷ in GenePattern.⁶⁸ Heatmap and Volcano plots were generated using R software.

Mass spectrometry-based proteome investigations: In Solution Digestion. OCI-Ly1 cell pellets were lysed at 4° C. in 8 M urea, 50 mM Tris-HCl pH 8.0, 150 mM NaCl, 1 mM EDTA, 2 μg/μl aprotinin (Sigma-Aldrich), 10 μg/μl leupeptin (Roche), and 1 mM phenylmethylsulfonyl fluoride (PMSF) (Sigma). Protein concentration was determined using a bicinchoninic acid (BCA) protein assay (Pierce). Proteins were reduced with 5 mM (DTT) for 45 min at room temperature (RT), followed by alkylation with 10 mM iodoacetamide for 30 min at room temperature in the dark. Urea concentration was reduced to 2 M with 50 mM Tris-HCl, pH 8. Samples were pre-digested for 2 h at 30° C. with endoproteinase Lys-C (Wako Laboratories) at an enzyme-to-substrate ratio of 1:50. Samples were digested overnight at 37° C. with sequencing grade trypsin (Promega) at an enzyme-to-substrate ratio of 1:50. Following overnight digest, samples were acidified with neat formic acid to a final concentration of 1%. Acidified samples were subsequently desalted on a 100 mg tC18 Sep-Pak SPE cartridge (Waters). Briefly, cartridges were conditioned with 1 mL of 100% MeCN, 1 mL of 50% MeCN/0.1% FA, and 4× with 1 mL of 0.1% TFA. The sample was loaded, and washed 3× with 1 mL of 0.1% TFA, lx with 1 mL of 1% FA, and eluted 2× with 600 μl of 50% MeCN/0.1% FA. Following desalting, 100 μg of the sample was dried to completion and stored at −80° C.

TMT labeling of peptides. Desalted peptides were labeled with TMT 10-plex isobaric mass tagging reagents (Thermo Fisher Scientific) as previously described⁶⁹. Each TMT reagent was resuspended in 41 μL of MeCN. Peptides were resuspended in 100 μL of 50 mM HEPES and combined with TMT reagent. Samples were incubated at RT for 1 h while shaking. The TMT reaction was quenched with 8 μL of 5% hydroxylamine at RT for 15 min with shaking. TMT labeled samples were combined, dried to completion, reconstituted in 100 μL of 0.1% FA, and desalted on StageTips or 100 mg SepPak columns as described above.

Basic Reverse Phase (bRP) Fractionation. The TMT labeled samples were fractionated using offline high pH reversed-phase chromatography (bRP) as previously described⁶⁹. Samples were fractionated using Zorbax 300 Extend C18 column (4.6×250 mm, 300 Å, 5 μm, Agilent) on an Agilent 1100 series high-pressure liquid chromatography (HPLC) system. Samples were reconstituted in 900 μL of 5 mM ammonium formate (pH 10.0)/2% (vol/vol) acetonitrile (MeCN) (bRP solvent A). Samples were injected with Solvent A at a flow rate of 1 mL/min and separated using a 96 min gradient. The gradient consisted of an initial increase to 16% solvent B (90% MeCN, 5 mM ammonium formate, pH 10), followed by 60 min linear gradient from 16% solvent B to 40% B and successive ramps to 44% and 60% at a flow rate of 1 mL/min. Fractions were collected in a 96-deep well plate (GE Healthcare) and pooled in a non-contiguous manner into final 24 proteome fractions. Pooled fractions were dried to completeness using a SpeedVac concentrator.

Liquid chromatography and mass spectrometry. Desalted peptides were resuspended in 9 μL of 3% MeCN/0.1% FA and analyzed by online nanoflow liquid chromatography tandem mass spectrometry (LC-MS/MS) using Q-Exactive+mass spectrometer (Thermo Fisher Scientific) coupled on-line to a Proxeon Easy-nLC 1200 (Thermo Fisher Scientific) as previously described⁶⁹. Briefly, 4 μL of each sample was loaded onto a microcapillary column (360 m outer diameter×75 m inner diameter) containing an integrated electrospray emitter tip (10 m), packed to approximately 22 cm with ReproSil-Pur C18-AQ 1.9 μm beads (Dr. Maisch GmbH) and heated to 50° C. Samples were analyzed with 110 min method. The 110 min method contained a mobile phase with a flow rate of 200 nL/min, comprised of 3% acetonitrile/0.1% formic acid (Solvent A) and 90% acetonitrile/0.1% formic acid (Solvent B), with the following gradient profile: (min:% B) 0:2; 1:6; 85:30; 94:60; 95:90; 100:90; 101:50; 110:50 (the last two steps at 500 nL/min flow rate). The Q-Exactive+MS was operated in the data-dependent mode acquiring HCD MS/MS scans (r=35,000) after each MS1 scan (r=70,000) on the 12 most abundant precursor ions using an MS1 target of 3×10⁶ and an MS2 target of 5×10⁴. The maximum ion time utilized for MS/MS scans was 120 ms; the HCD-normalized collision energy was set to 30; the dynamic exclusion time was set to 20 s, isotope exclusion function was enabled, and peptide match function was set to preferred. Charge exclusion was enabled for charge states that were unassigned, 1 and >6.

Data Analysis. All data were analyzed using Spectrum Mill software package v 6.1 pre-release (Agilent Technologies). Similar MS/MS spectra acquired on the same precursor m/z within +/−60 s were merged. MS/MS spectra were excluded from searching if they were not within the precursor MH+ range of 750-4000 Da or if they failed the quality filter by not having a sequence tag length >0. MS/MS spectra were searched against UniProt human database. All spectra were allowed +/−20 ppm mass tolerance for precursor and product ions, 30% minimum matched peak intensity, and “trypsin allow P” enzyme specificity with up to 2 missed cleavages. The fixed modifications were carbamidomethylation at cysteine, and TMT at N-termini and internal lysine residues. Variable modifications included oxidized methionine and N-terminal protein acetylation. Individual spectra were automatically designated as confidently assigned using the Spectrum Mill autovalidation module. Specifically, a target-decoy based FDR scoring threshold criteria via a two-step auto threshold strategy at the spectral and protein levels was used. First, peptide mode was set to allow automatic variable range precursor mass filtering with score thresholds optimized to yield a spectral level FDR of <1.2%. A protein polishing autovalidation was applied to further filter the peptide spectrum matches using a target protein-level FDR threshold of 0. Following autovalidation, a protein-protein comparison table was generated, which contained experimental ratios. For all experiments, non-human contaminants and reversed hits were removed. Furthermore, data were filtered to only consider proteins with 2 or more unique peptides and was median normalized.

For statistical analysis, a moderated t-test was implemented in R-Shiny using the limma R library. Correction for multiple testing was performed using the Benjamini-Hochberg false discovery rate method.

Measurement of oxygen consumption and extracellular acidification rate. For each condition, 1.25×10⁵ cells were counted and plated on a Seahorse plate coated with Cell-Tak (Corning Life Sciences) in Seahorse XF DMEM media (Agilent). OCR and ECAR were recorded using a Seahorse XF96 Analyzer (Agilent) for 24 consecutive measurements. OCR is measured before and after the addition of inhibitors to assess mitochondrial function by deriving several parameters of mitochondrial respiration: (i) basal respiration, (ii) ATP-linked respiration and proton leak respiration (after 3 μM oligomycin [Sigma], a complex V inhibitor) and (iii) maximal respiration (after 1 μM carbonyl cyanide m-chlorophenyl hydrazine (CCCP) [Sigma], a protonophore). Mitochondrial respiration is finally inhibited by 1 μM antimycin A (Sigma), a complex III inhibitor.

To measure the effect of venetoclax on OCR/ECAR, cells were pre-treated for 1 h with 10 M of zVAD-FMK (Abcam) or left untreated and OCR/ECAR were recorded for 4 measurements before injection of venetoclax at a final concentration of 100 nM. DMSO injection was used as control.

Mitochondrial DNA copy number. Relative mtDNA copy number was determined using a multiplexed qPCR assay previously reported⁷⁰.

Measurement of mitochondrial superoxide levels. To measure relative levels of mitochondrial superoxide, cells were stained with 5 μM MitoSOX Red (Invitrogen, cat #M36008) for 10 min at 37° C. Cells were then washed three times with the media. Using a flow cytometer (FACSymphony, BD), MitoSOX Red was excited at 488 nm and fluorescence emission at 610/20 nm (BB630) was measured. Relative fluorescence intensity from biological triplicate of 10,000 cells were used as an indicator of mitochondrial superoxide levels.

Mitochondrial membrane potential. Cells were stained with 2.5 μM JC-1 (ThermoFisher Scientific, cat #T3168) for 30 min at 37° C. Cells were then washed three times with the media and subjected to the flow cytometry (FACS-Canto) following manufacturer instruction. Briefly, JC-1 was excited at 488 nm and its emission at both 525 nm (FITC-A) and 585 nm (PE-A) were measured. By comparing the ratio of emission at 585 nm/525 nm, relative levels of mitochondrial membrane potential were determined from 10,000 cells in biological triplicate.

Statistical analysis. Data analyses were carried out using GraphPad Prism version 7 and R software. The data are summarized as mean±s.e.m. or s.d. as indicated, and are presented as individual values as scatter plots with column bar graphs and were analyzed using Student's t-tests (two-sided). One-way ANOVA was used to compare three or more groups in time point analyses. Differences were considered significant when P<0.05.

Synergy analysis for venetoclax drug combinations were performed using the Chou-Talalay method based on the median-effect principle.⁷¹ Combination index values and isobolograms were generated using CompuSyn.

### REFERENCES

1. Tsujimoto, Y., Cossman, J., Jaffe, E. & Croce, C. M. Involvement of the bcl-2 gene in human follicular lymphoma. Science 228, 1440-1443 (1985).
2. Kridel, R., Sehn, L. H. & Gascoyne, R. D. Pathogenesis of follicular lymphoma. J. Clin. Invest. 122, 3424-3431 (2012).
3. Cimmino, A. et al. miR-15 and miR-16 induce apoptosis by targeting BCL2. Proc. Natl. Acad. Sci. U.S.A 102, 13944-13949 (2005).
4. Roberts, A. W. et al. Targeting BCL2 with Venetoclax in Relapsed Chronic Lymphocytic Leukemia. N. Engl. J. Med. 374, 311-322 (2016).
5. Del Gaizo Moore, V., Schlis, K. D., Sallan, S. E., Armstrong, S. A. & Letai, A. BCL-2 dependence and ABT-737 sensitivity in acute lymphoblastic leukemia. Blood 111, 2300-2309 (2008).
6. Anderson, M. A. et al. Clinicopathological features and outcomes of progression of CLL on the BCL2 inhibitor venetoclax. Blood 129, 3362-3370 (2017).
7. Mato, A. R. et al. Real world outcomes and management strategies for venetoclax-treated chronic lymphocytic leukemia patients in the United States. Haematologica (2018). doi:10.3324/haematol.2018.193615
8. Doench, J. G. et al. Optimized sgRNA design to maximize activity and minimize off-target effects of CRISPR-Cas9. Nat. Biotechnol. 34, 184-191 (2016).
9. Meissner, B. et al. The E3 ubiquitin ligase UBR5 is recurrently mutated in mantle cell lymphoma. Blood 121, 3161-3164 (2013).
10. Love, C. et al. The genetic landscape of mutations in Burkitt lymphoma. Nat. Genet. 44, 1321-1325 (2012).
11. Weniger, M. A. & Kuppers, R. NF-κB deregulation in Hodgkin lymphoma. Semin. Cancer Biol. 39, 32-39 (2016).
12. Lark, D. S. et al. Protein Kinase A Governs Oxidative Phosphorylation Kinetics and Oxidant Emitting Potential at Complex I. Front. Physiol. 6, 332 (2015).
13. Lin, S.-C. & Hardie, D. G. AMPK: Sensing Glucose as well as Cellular Energy Status. Cell Metab. 27, 299-313 (2018).
14. Deng, J. et al. BH3 profiling identifies three distinct classes of apoptotic blocks to predict response to ABT-737 and conventional chemotherapeutic agents. Cancer Cell 12, 171-185 (2007).
15. Yecies, D., Carlson, N. E., Deng, J. & Letai, A. Acquired resistance to ABT-737 in lymphoma cells that up-regulate MCL-1 and BFL-1. Blood 115, 3304-3313 (2010).
16. Kotschy, A. et al. The MCL1 inhibitor S63845 is tolerable and effective in diverse cancer models. Nature 538, 477-482 (2016).
17. Bott, A. J. et al. Oncogenic Myc Induces Expression of Glutamine Synthetase through Promoter Demethylation. Cell Metab. 22, 1068-1077 (2015).
18. Guo, B., Huang, X., Lee, M. R., Lee, S. A. & Broxmeyer, H. E. Antagonism of PPAR-γ signaling expands human hematopoietic stem and progenitor cells by enhancing glycolysis. Nat. Med. (2018). doi:10.1038/nm.4477
19. Arroyo, J. D. et al. A Genome-wide CRISPR Death Screen Identifies Genes Essential for Oxidative Phosphorylation. Cell Metab. 24, 875-885 (2016).
20. Chan, L. N. et al. Metabolic gatekeeper function of B-lymphoid transcription factors. Nature 542, 479-483 (2017).
21. Young, V. J., Ahmad, S. F., Brown, J. K., Duncan, W. C. & Horne, A. W. ID2 mediates the transforming growth factor-β1-induced Warburg-like effect seen in the peritoneum of women with endometriosis. Mol. Hum. Reprod. 22, 648-654 (2016).
22. Zhang, Z., Rahme, G. J., Chatterjee, P. D., Havrda, M. C. & Israel, M. A. ID2 promotes survival of glioblastoma cells during metabolic stress by regulating mitochondrial function. Cell Death Dis. 8, e2615 (2017).
23. Chapuy, B. et al. Molecular subtypes of diffuse large B cell lymphoma are associated with distinct pathogenic mechanisms and outcomes. Nat. Med 24, 679-690 (2018).
24. Fresquet, V., Rieger, M., Carolis, C., Garcia-Barchino, M. J. & Martinez-Climent, J. A. Acquired mutations in BCL2 family proteins conferring resistance to the BH3 mimetic ABT-199 in lymphoma. Blood 123, 4111-4119 (2014).
25. Burger, J. A. et al. Clonal evolution in patients with chronic lymphocytic leukaemia developing resistance to BTK inhibition. Nat. Commun. 7, 11589 (2016).
26. Song, J. H., Kandasamy, K. & Kraft, A. S. ABT-737 induces expression of the death receptor 5 and sensitizes human cancer cells to TRAIL-induced apoptosis. J. Biol. Chem. 283, 25003-25013 (2008).
27. Herling, C. D. et al. Clonal dynamics towards the development of venetoclax resistance in chronic lymphocytic leukemia. Nat. Commun. 9, 727 (2018).
28. Gorre, M. E. et al. Clinical resistance to STI-571 cancer therapy caused by BCR-ABL gene mutation or amplification. Science 293, 876-880 (2001).
29. Ellis, L. M. & Hicklin, D. J. Resistance to Targeted Therapies: Refining Anticancer Therapy in the Era of Molecular Oncology. Clin. Cancer Res. Off J. Am. Assoc. Cancer Res. 15, 7471-7478 (2009).
30. Woyach, J. A. et al. Resistance mechanisms for the Bruton's tyrosine kinase inhibitor ibrutinib. N. Engl. J. Med 370, 2286-2294 (2014).
31. Konopleva, M. et al. Mechanisms of apoptosis sensitivity and resistance to the BH3 mimetic ABT-737 in acute myeloid leukemia. Cancer Cell 10, 375-388 (2006).
32. Lin, K. H. et al. Targeting MCL-1/BCL-XL Forestalls the Acquisition of Resistance to ABT-199 in Acute Myeloid Leukemia. Sci. Rep. 6, 27696 (2016).
33. Harrison, L. R. E. et al. Hypoxic human cancer cells are sensitized to BH-3 mimetic-induced apoptosis via downregulation of the Bcl-2 protein Mcl-1. J. Clin. Invest. 121, 1075-1087 (2011).
34. Meynet, O. et al. Caloric restriction modulates Mcl-1 expression and sensitizes lymphomas to BH3 mimetic in mice. Blood 122, 2402-2411 (2013).
35. Kerkela, R. et al. Sunitinib-induced cardiotoxicity is mediated by off-target inhibition of AMP-activated protein kinase. Clin. Transl. Sci. 2, 15-25 (2009).
36. Oppermann, S. et al. High-content screening identifies kinase inhibitors that overcome venetoclax resistance in activated CLL cells. Blood 128, 934-947 (2016).
37. Mootha, V. K. et al. A reversible component of mitochondrial respiratory dysfunction in apoptosis can be rescued by exogenous cytochrome c. EMBO J. 20, 661-671 (2001).
38. Jiang, X. et al. A Small Molecule That Protects the Integrity of the Electron Transfer Chain Blocks the Mitochondrial Apoptotic Pathway. Mol. Cell 63, 229-239 (2016).
39. Chen, Z. X. & Pervaiz, S. Bcl-2 induces pro-oxidant state by engaging mitochondrial respiration in tumor cells. Cell Death Differ. 14, 1617-1627 (2007).
40. Lagadinou, E. D. et al. BCL-2 inhibition targets oxidative phosphorylation and selectively eradicates quiescent human leukemia stem cells. Cell Stem Cell 12, 329-341 (2013).
41. Gimenez-Cassina, A. & Danial, N. N. Regulation of mitochondrial nutrient and energy metabolism by BCL-2 family proteins. Trends Endocrinol. Metab. TEM 26, 165-175 (2015).
42. Gross, A. & Katz, S. G. Non-apoptotic functions of BCL-2 family proteins. Cell Death Differ. 24, 1348-1358 (2017).
43. Shalem, O. et al. Genome-scale CRISPR-Cas9 knockout screening in human cells. Science 343, 84-87 (2014).
44. Johannessen, C. M. et al. A melanocyte lineage program confers resistance to MAP kinase pathway inhibition. Nature 504, 138-142 (2013).
45. Rotem, A. et al. Alternative to the soft-agar assay that permits high-throughput drug and genetic screens for cellular transformation. Proc. Natl. Acad. Sci. U.S.A 112, 5708-5713 (2015).
46. Sanjana, N. E., Shalem, O. & Zhang, F. Improved vectors and genome-wide libraries for CRISPR screening. Nat. Methods 11, 783-784 (2014).
47. Doench, J. G. et al. Rational design of highly active sgRNAs for CRISPR-Cas9-mediated gene inactivation. Nat. Biotechnol. 32, 1262-1267 (2014).
48. Fisher, S. et al. A scalable, fully automated process for construction of sequence-ready human exome targeted capture libraries. Genome Biol. 12, R1 (2011).
49. Li, H. & Durbin, R. Fast and accurate long-read alignment with Burrows-Wheeler transform. Bioinforma. Oxf. Engl. 26, 589-595 (2010).
50. Costello, M. et al. Discovery and characterization of artifactual mutations in deep coverage targeted capture sequencing data due to oxidative DNA damage during sample preparation. Nucleic Acids Res. 41, e67 (2013).
51. Cibulskis, K. et al. ContEst: estimating cross-contamination of human samples in next-generation sequencing data. Bioinforma. Oxf. Engl. 27, 2601-2602 (2011).
52. Berger, M. F. et al. The genomic complexity of primary human prostate cancer. Nature 470, 214-220 (2011).
53. Chapman, M. A. et al. Initial genome sequencing and analysis of multiple myeloma. Nature 471, 467-472 (2011).
54. Saunders, C. T. et al. Strelka: accurate somatic small-variant calling from sequenced tumor-normal sample pairs. Bioinforma. Oxf. Engl. 28, 1811-1817 (2012).
55. Robinson, J. T. et al. Integrative genomics viewer. Nat. Biotechnol. 29, 24-26 (2011).
56. Taylor-Weiner, A. et al. DeTiN: overcoming tumor-in-normal contamination. Nat. Methods 15, 531-534 (2018).
57. Wang, L. et al. SF3B1 and other novel cancer genes in chronic lymphocytic leukemia. N. Engl. J. Med. 365, 2497-2506 (2011).
58. Quesada, V. et al. Exome sequencing identifies recurrent mutations of the splicing factor SF3B1 gene in chronic lymphocytic leukemia. Nat. Genet. 44, 47-52 (2012).
59. Landau, D. A. et al. Evolution and Impact of Subclonal Mutations in Chronic Lymphocytic Leukemia. Cell 152, 714-726 (2013).
60. Puente, X. S. et al. Non-coding recurrent mutations in chronic lymphocytic leukaemia. Nature 526, 519-524 (2015).
61. Landau, D. A. et al. Mutations driving CLL and their evolution in progression and relapse. Nature 526, 525-530 (2015).
62. Stachler, M. D. et al. Paired exome analysis of Barrett's esophagus and adenocarcinoma. Nat. Genet. 47, 1047-1055 (2015).
63. Carter, S. L. et al. Absolute quantification of somatic DNA alterations in human cancer. Nat. Biotechnol. 30, 413-421 (2012).
64. Dobin, A. et al. STAR: ultrafast universal RNA-seq aligner. Bioinforma. Oxf. Engl. 29, 15-21 (2013).
65. Li, B. & Dewey, C. N. RSEM: accurate transcript quantification from RNA-Seq data with or without a reference genome. BMC Bioinformatics 12, 323 (2011).
66. Love, M. I., Huber, W. & Anders, S. Moderated estimation of fold change and dispersion for RNA-seq data with DESeq2. Genome Biol. 15, 550 (2014).
67. Subramanian, A. et al. Gene set enrichment analysis: a knowledge-based approach for interpreting genome-wide expression profiles. Proc. Natl. Acad. Sci. U.S.A 102, 15545-15550 (2005).
68. Reich, M. et al. GenePattern 2.0. Nat. Genet. 38, 500-501 (2006).
69. Mertins, P. et al. Reproducible workflow for multiplexed deep-scale proteome and phosphoproteome analysis of tumor tissues by liquid chromatography-mass spectrometry. Nat. Protoc. (2018). doi:10.1038/s41596-018-0006-9
70. Bao, X. R. et al. Mitochondrial dysfunction remodels one-carbon metabolism in human cells. eLife 5, (2016).
71. Chou, T.-C. Drug Combination Studies and Their Synergy Quantification Using the Chou-Talalay Method. Cancer Res. 70, 440-446 (2010).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of inhibiting tumor growth of a BCL-2-driven cancer in a subject in need thereof, increasing sensitivity of a cell or population of cells to a BCL-2 inhibitor, or decreasing a BCL-2 inhibitor resistance signature of a cell or population of cells comprising administering to the subject, cell or population of cells:
one or more agents capable of inhibiting the oxidative phosphorylation system (OXPHOS); or
one or more agents that induces or enhances expression, activity, and/or function of one or more BCL-2 inhibitor resistance signature genes selected from the group consisting of those listed in Table 1, downregulated genes in Table 3, and/or downregulated genes in Table 4: or an agent that inhibits expression, activity, and/or function of one or more BCL-2 inhibitor resistance signature genes selected from the group consisting of those listed in Table 2, upregulated genes in Table 3, and/or upregulated genes in Table 4, preferably, a therapeutically effective amount of one or more agents; or
a combination therapy comprising an inhibitor of BCL-2 and one or more NF kappa B inhibitors.

2. The method of claim 1, wherein the method comprises administering to the subject a combination therapy comprising an inhibitor of BCL-2 and one or more inhibitors selected from the group consisting of an AMPK inhibitor and mitochondrial electron transport chain (mETC) inhibitor, preferably,
wherein the BCL-2 inhibitor is Venetoclax; and/or
wherein the AMPK inhibitor is dorsomorphin (compound C); and/or
wherein the mitochondrial electron transport chain (mETC) inhibitor comprises oligomycin or antimycin.

3. (canceled)

4. (canceled)

5. (canceled)

6. (canceled)

7. The method of claim 1, wherein the one or more agents increase expression, activity, and/or function of one or more target genes or one or more products of one or more target genes selected from the group consisting of:
a. PMAIP1, BAX, NFKBIA, IKZF5, BAK1, ID3, EP300, ZEB2, NFIA, BCL2L11 and OTUD5; or
b. FNBP1, CD9, PLXNB2, TTC39C and DENND3; or
c. XBP1, CYBB, PAG1 and DIRAS1; or
d. CD9, PLXNB2, TTC39C, DENND3, ICAM1, GNG7, ID2, FNBP1, FBP1, ACY3, CDKN1A, GALM, PTK2 and CYBB.

8. The method of claim 1, wherein the one or more agents decrease expression, activity, and/or function of one or more target genes or one or more products of one or more target genes selected from the group consisting of:
a. BCL2L1, BCL2L2, BCL2, MCL1, SRPX, RNF26, HSPB9, OR1S2, ADIPOQ, PIGF, CSGALNACT1, OTUD6A, SLC25A3, PRKAR2B, DNM2, SPHAR, APOBEC3C, RPL17, INMT, THADA, SBNO2, PRKAA2, BRMS1L, TRNAU1AP, CNNM3, ADAM33, PRKD2, FCHSD2, LOC399886, BABAM1, C1orf146, LMAN2L, ZNF460, TEX2, YRDC, ARHGAP11A, SPEG, FBXO9, USP54, SLC22A6, RPS4Y1, FAM71C, SH3BGRL2, HCRTR1, BST1, PHF10, UCKL1, ATG5, RPS15A, CDCl20B, PPIE, TUT1, RPL36, HSD11B1L, MTERF4, PTS, S1PR4, HJURP, HMMR, BOLA2, DNASE1L1, OSGEP, TMBIM4, BTNL3, CHRM3, FBX015, KLK8, ASPN, STYK1 and SRSF6; or
b. SYT11, PARM1, ROBO2, CD48, FCRL1 and MCL1; or
c. PLCL2, KCNA3, TNFRSF21, CYP2U1, TRAM2 and RAPGEF5; or
d. TSTD1, DNAJC12, TRAF3IP3, OPTN, DOCK10, PYHINI, CD48, P4HA2, PLCL2, AOX1, CDK6, GATM, GLUL, PAPSS1, MCL1 and GATM.

9. The method of claim 1, wherein the tumor overexpresses BCL-2; and/or
wherein the tumor is resistant to an inhibitor of BCL-2; and/or
wherein the tumor is resistant to Venetoclax.

10. (canceled)

11. (canceled)

12. The method of claim 1, further comprising administering to said subject a therapeutically effective amount of an inhibitor of BCL-2, preferably, wherein the inhibitor of BCL-2 is Venetoclax.

13. (canceled)

14. (canceled)

15. The method of claim 1, wherein the NF kappa B inhibitor is selected from the group consisting of denosumab, disulfiram, olmesartan, dithiocarbamates, anatabine, BAY 11-7082 and iguratimod.

16. (canceled)

17. (canceled)

18. (canceled)

19. The method of claim 1, wherein the one or more agents enhance expression, activity, and/or function of one or more genes selected from the group consisting of:
a) PMAIP1, BAX, BAK1, or BCL-2L11,
b) NFKBIA, IKZF5, ID3, EP300, NFIA, OTUD5, or UBR5; or
c) FNBP1, CD9, PLXNB2, TTC39C, DENND3, XBP1, CYBB, PAG1, DIRAS1, ICAM1, GNG7, ID2, FBP1, ACY3, CDKN1A, GALM or PTK2; or
decrease expression, activity, and/or function of one or more genes selected from the group consisting of:
a) BCL2L1, BCL2L12, BCL2 or MCL1,
b) ADIPOQ, PRKAR2B, PRKAA2, SLC25A3, RFN26, DNM2, PRKD2, ATG5, RPL17, RPS4Y1, RPS15A, OUTUD6A, FBXO9, or USP54, or
c) SYT11, PARM1, ROBO2, CD48, FCRL1, MCL1, PLCL2, KCNA3, TNFRSF21, CYP2U1, TRAM2, RAPGEF5, TSTD1, DNAJC12, TRAF3IP3, OPTN, DOCK10, PYHINI, CD48, P4HA2, AOX1, CDK6, GATM, GLUL, PAPSS1 or GATM.

20. A method of screening for one or more agents that increases a BCL-2 inhibitor sensitive signature or decreases a BCL-2 inhibitor resistance signature of a cell or a population of cells that expresses BCL-2 comprising:
delivering to the cell one or more candidate agents and selecting one or more agents that increases expression, activity, and/or function of one or more target genes or one or more products of one or more genes selected from the group consisting of those listed in Table 1, downregulated genes in Table 3, and/or downregulated genes in Table 4; or
decreases expression, activity, and/or function of one or more target genes or one or more products of one or more target genes selected from the group consisting of those listed in Table 2, upregulated genes in Table 3, and/or upregulated genes in Table 4.

21. The method of claim 20, wherein the one or more candidate agents increase expression, activity, and/or function of one or more target genes or one or more products of one or more target genes which comprise inhibitors of the NF-Kappa B pathway, lymphoid transcription factors and modulators, ubiquitination components, and/or pro-apoptotic BCL-2 family proteins; or
wherein the one or more candidate agents decrease expression, activity, and/or function of one or more target genes or one or more products of one or more target genes which comprise energy-stress sensor signaling pathway components, a mitochondrial energy metabolism component, vesicle transport/autophagy components, ribosomal components, and/or ubiquitination components; or
wherein the one or more candidate agents increase expression, activity, and/or function of one or more target genes or one or more products of one or more target genes selected from the group consisting of:
a. PMAIP1, BAX, NFKBIA, IKZF5, BAK1, ID3, EP300, ZEB2, NFIA, BCL2L11 and OTUD5; or
b. FNBP1, CD9, PLXNB2, TTC39C and DENND3; or
c. XBP1, CYBB, PAG1 and DIRAS1: or
d. CD9, PLXNB2, TTC39C, DENND3, ICAM1, GNG7, ID2, FNBP1, FBP1, ACY3, CDKN1A, GALM, PTK2 and CYBB; or
wherein the one or more candidate agents decrease expression, activity, and/or function of one or more target genes or one or more products of one or more target genes selected from the group consisting of:
a. BCL2L1, BCL2L2, BCL2, MCL1, SRPX, RNF26, HSPB9, OR1S2, ADIPOQ, PIGF, CSGALNACT1, OTUD6A, SLC25A3, PRKAR2B, DNM2, SPHAR, APOBEC3C, RPL17, INMT, THADA, SBNO2, PRKAA2, BRMS1L, TRNAU1AP, CNNM3, ADAM33, PRKD2, FCHSD2, LOC399886, BABAM1, C1orf146, LMAN2L, ZNF460, TEX2, YRDC, ARHGAP11A, SPEG, FBXO9, USP54, SLC22A6, RPS4Y1, FAM71C, SH3BGRL2, HCRTR1, BST1, PHF10, UCKL1, ATG5, RPS15A, CDC20B, PPIE, TUT1, RPL36, HSD11B1L, MTERF4, PTS, S1PR4, HJURP, HMMR, BOLA2, DNASE1L1, OSGEP, TMBIM4, BTNL3, CHRM3, FBX015, KLK8, ASPN, STYK1 and SRSF6; or
b. SYT11, PARM1, ROBO2, CD48, FCRL1 and MCL1; or
c. PLCL2, KCNA3, TNFRSF21, CYP2U1, TRAM2 and RAPGEF5; or
d. TSTD1, DNAJC12, TRAF3IP3, OPTN, DOCK10, PYHINI, CD48, P4HA2, PLCL2, AOX1, CDK6, GATM, GLUL, PAPSS1, MCL1 and GATM.

22. (canceled)

23. (canceled)

24. (canceled)

25. The method of claim 20, wherein the cell or population of cells overexpresses BCL-2.

26. The method of claim 20, wherein the method further comprises exposing the cell or population of cells to an agent that modulates the expression or activity of at least one BCL-2 antagonist of cell death (BAD) pathway component, preferably, wherein the method further comprises exposing the cell or population of cells to an agent that inhibits BCL-2, more preferably, wherein the agent that inhibits BCL-2 is Venetoclax.

27. (canceled)

28. (canceled)

29. The method of claim 1, wherein the agent is a small molecule, small molecule degrader, genetic modifying agent, antibody, antibody fragment, antibody-like protein scaffold, aptamer, protein, or any combination thereof.

30. The method of claim 29, wherein the genetic modifying agent comprises a CRISPR system, RNAi system, a zinc finger nuclease system, a TALE system, or a meganuclease, preferably,
wherein the CRISPR system comprises a Class 2, Type II, V, or VI CRISPR-Cas system; or
wherein the CRISPR system comprises a dCas fused or otherwise linked to a nucleotide deaminase, more preferably, wherein the nucleotide deaminase is a cytidine deaminase or an adenosine deaminase.

31. (canceled)

32. (canceled)

33. (canceled)

34. A method of detecting a BCL-2 inhibitor resistance signature in a subject in need thereof comprising detecting in a tumor sample obtained from the subject the expression of one or more genes selected from the group consisting of those listed in Table 1, Table 2, Table 3, and/or Table 4.

35. The method of claim 34, wherein the genes selected from the group consisting of:
a. PMAIP1, BAX, NFKBIA, IKZF5, BAK1, ID3, EP300, ZEB2, NFIA, BCL2L11 and OTUD5; or
b. FNBP1, CD9, PLXNB2, TTC39C and DENND3; or
c. XBP1, CYBB, PAG1 and DIRAS1; or
d. CD9, PLXNB2, TTC39C, DENND3, ICAM1, GNG7, ID2, FNBP1, FBP1, ACY3, CDKN1A, GALM, PTK2 and CYBB,
are downregulated as compared to a reference value; or
wherein the genes selected from the group consisting of:
a. BCL2L1, BCL2L2, BCL2, MCL1, SRPX, RNF26, HSPB9, OR1S2, ADIPOQ, PIGF, CSGALNACT1, OTUD6A, SLC25A3, PRKAR2B, DNM2, SPHAR, APOBEC3C, RPL17, INMT, THADA, SBNO2, PRKAA2, BRMS1L, TRNAU1AP, CNNM3, ADAM33, PRKD2, FCHSD2, LOC399886, BABAM1, C1orf146, LMAN2L, ZNF460, TEX2, YRDC, ARHGAP11A, SPEG, FBXO9, USP54, SLC22A6, RPS4Y1, FAM71C, SH3BGRL2, HCRTR1, BST1, PHF10, UCKL1, ATG5, RPS15A, CDC20B, PPIE, TUT1, RPL36, HSD11B1L, MTERF4, PTS, S1PR4, HJURP, HMMR, BOLA2, DNASE1L1, OSGEP, TMBIM4, BTNL3, CHRM3, FBXO15, KLK8, ASPN, STYK1 and SRSF6; or
b. SYT11, PARM1, ROBO2, CD48, FCRL1 and MCL1; or
c. PLCL2, KCNA3, TNFRSF21, CYP2U1, TRAM2 and RAPGEF5; or
d. TSTD1, DNAJC12, TRAF3IP3, OPTN, DOCK10, PYHINI, CD48, P4HA2, PLCL2, AOX1, CDK6, GATM, GLUL, PAPSS1, MCL1 and GATM,
are upregulated as compared to a reference value.

36. (canceled)

37. The method of claim 34, wherein if a BCL-2 inhibitor resistance signature is detected the method further comprises administering a treatment to the subject according to claim 1.

38. A method of identifying a signature gene, a gene signature, or other genetic element associated with a BCL-2 family function, activity or phenotype comprising:
a) contacting a cell or population of cells with an agent that inhibits an anti-apoptotic BCL-2 family protein or a gene that encodes the protein; and
b) identifying one or more gene loci whose activity is modulated by step (a);
thereby identifying a signature gene, a gene signature, or other genetic element associated with a BCL-2 family function.

39. The method of claim 38, wherein the cell or population of cells comprises a Cas protein or nucleic acid encoding the Cas protein and one or more guides or nucleic acids encoding the one or more guides,
wherein the guide(s) target one or more nucleic acid(s) in the cell or population of cells,
whereby one or more nucleic acid(s) in the cell or population of cells is modified,
whereby the viability of a cell or population of cells comprising the one or more modified nucleic acid(s) is modulated; and/or
wherein the cell or population of cells comprises nucleic acids modified by a CRISPR-Cas system comprising a Cas protein and one or more guides; and/or
wherein the viability of the cell or cell population comprising the one or more modified nucleic acid(s) is correlated with representation of one or more of the one or more guides; and/or
wherein the cell or population of cells comprises one or more gene knock-outs; and/or
wherein the CRISPR-Cas system comprises a Cas9; and/or
wherein the BCL-2 family protein is BCL-2.

40. (canceled)

41. (canceled)

42. (canceled)

43. (canceled)

44. (canceled)

45. A kit comprising reagents to detect at least one gene or gene product according to claim 34.


SEQUENCE LISTING (4 sequences)

SEQ ID NO: 1 - DNA, 20 bp, Artificial Sequence
  Synthetic Oligonucleotide
tgacaagttccggagtgagc

SEQ ID NO: 2 - DNA, 20 bp, Artificial Sequence
  Synthetic Oligonucleotide
cggtatcagcgcttcctcat

SEQ ID NO: 3 - protein, 288 aa, Artificial Sequence
  Synthetic Peptide
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 4 - protein, 183 aa, Artificial Sequence
  Synthetic Peptide
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS